(19) United States
(12) Patent Application Publication
(10) Pub. No.: US 2012/0121540 A1
(43) Pub. Date: May 17, 2012

(54) Certain Nitrogen Containing Bicyclic Chemical Entities For Treating Viral Infections

(76) Inventors: Franz Ulrich Schmitz, Mill Valley, CA (US); Vincent W.F Tai, San Mateo, CA (US); Roopa Ral, San Carlos, CA (US); Christopher Don Roberts, Belmont, CA (US); Ali Dehghani Mohammad Abadi, Campbell, CA (US); Subramanian Baskaran, Foster City, CA (US); Irina Slobodov, San Mateo, CA (US); Jack Maung, Daly City, CA (US); Martin Leon Neitzel, Pacifica, CA (US)

(21) Appl. No.: 12/672,942

(22) PCT Filed: Aug. 8, 2008
(86) PCT No.: PCT/US08/09606

§ 371 Date: Mar. 31, 2010

Related U.S. Application Data
(60) Provisional application No. 61/041,084, filed on Mar. 31, 2008.

(30) Foreign Application Priority Data
Aug. 10, 2007 (US) 60964223

(51) Int. Cl. A61K 31/437; C07D 409/12; A61K 31/404; C07D 409/14; A61K 31/5377; C07D 519/00; C07D 487/04; A61K 31/5025; A61K 31/496; A61K 31/506; A61K 31/497; C07D 453/04; A61K 31/46; A61K 38/21; A61K 31/444; C07D 405/10; A61K 31/4184; C07D 405/14; A61K 31/4545; C07D 471/04

(52) U.S. Cl. 424/85.4; 546/121; 514/300; 546/118; 514/303; 548/467; 514/414; 544/127; 514/233.2; 544/236; 514/248; 544/362; 514/253.04; 514/210.18; 544/297; 514/275; 544/357; 514/252.11; 544/295; 514/252.14; 544/333; 514/256; 548/304.7; 514/394; 548/306.1; 514/210.21; 544/263; 514/259.31; 544/281; 514/259.3

(57) ABSTRACT

Provided are certain chemical entities, pharmaceutical compositions, and methods of treatment of a member of the flaviviradae family of viruses such as hepacivirus (Hepatitis C or HCV).

This application claims the benefit of U.S. provisional patent application No. 61/041,084, filed 31 Mar. 2008 and of U.S. provisional patent application No. 60/964,223, filed 10 Aug. 2007, each of which is incorporated herein by reference.

Provided are certain chemical entities, pharmaceutical compositions and methods of treatment of a member of the flaviviradae family of viruses such as hepacivirus (Hepatitis C or HCV).

The Flaviviridae family of viruses is composed of three genera: pestivirus, flavivirus and hepacivirus (hepatitis C virus). Of these genera, flaviviruses and hepaciviruses represent important pathogens of man and are prevalent throughout the world. There are 38 flaviviruses associated with human disease, including the dengue fever viruses, yellow fever virus, and Japanese encephalitis virus. Flaviviruses cause a range of acute febrile illnesses and encephalitic and hemorrhagic diseases. Hepaciviruses currently infect approximately 2 to 3% of the world population and cause persistent infections leading to chronic liver disease, cirrhosis, hepatocellular carcinoma and liver failure. Human pestiviruses have not been as extensively characterized as the animal pestiviruses. However, serological surveys indicate considerable pestivirus exposure in humans. Pestivirus infections in man have been implicated in several diseases including, but not limited to, congenital brain injury, infantile gastroenteritis and chronic diarrhea in human immunodeficiency virus (HIV).

HCV is a major causative agent for post-transfusion and for sporadic hepatitis. Infection by HCV is insidious in a high proportion of chronically infected (and infectious) carriers who may not experience clinical symptoms for many years.

At present, the only acceptable treatment for chronic HCV is interferon (IFN-alpha) and/or ribavirin and this requires at least six (6) months of treatment, which can reduce the viral load and also improve liver function in some people.

IFN-alpha belongs to a family of naturally occurring small proteins with characteristic biological effects such as antiviral, immunoregulatory and anti-tumoral activities. IFN-alpha is an important regulator of immunological control. Treatment of HCV with interferon, however, has limited long term efficacy with a response rate about 25%. In addition, treatment of HCV with interferon has frequently been associated with adverse side effects such as fatigue, fever, chills, headache, myalgias, arthralgias, mild alopecia, psychiatric effects and associated disorders, autoimmune phenomena and associated disorders and thyroid dysfunction.

Ribavirin (1-P-D-ribofuranosyl-1H-1,2,-4-triazole-3-carboxamide), an inhibitor of inosine 5′-monophosphate dehydrogenase (IMPDH), enhances the efficacy of IFN-alpha in the treatment of HCV. Despite the introduction of Ribavirin, up to 50% of the patients do not eliminate the virus with the current standard therapy of interferon-alpha (IFN) and Ribavirin. Ribavirin causes significant hemolysis in 10-20% of patients treated at currently recommended doses, and the drug is both teratogenic and embryotoxic. By now, standard therapy of chronic hepatitis C has been changed to the combination of PEG-IFN (pegylated interferon) plus ribavirin which leads only to small improvement.

Other approaches are being taken to combat the virus. They include, for example, application of antisense oligonucleotides or ribozymes for inhibiting HCV replication. Furthermore, low-molecular weight compounds that directly inhibit HCV proteins and interfere with viral replication are considered as attractive strategies to control HCV infection. Among non-structral viral proteins, NS3/4a serine protease, NS5b RNA dependent RNA polymerase are considered as prime targets for new drugs.

There is a need for the development of new compounds that combat hepacivirus. There remains a need for agents with stronger response rates and fewer side effects in terms of relief of symptoms, safety, and patient mortality, both short-term and long-term and an improved therapeutic index.

Provided is at least one chemical entity selected from compounds of Formula 1:

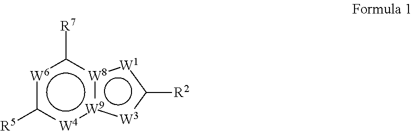

and pharmaceutically acceptable salts thereof, wherein

W¹ is selected from CR¹ and NR¹;

W³ is selected from CR³ and NR³;

W⁴ is selected from CR⁴ and N;

W⁶ is selected from CR⁶ and N;

W⁸ is selected from C and N;

W⁹ is selected from C and N;

R¹ is absent or is selected from hydrogen, halogen, optionally substituted alkyl, optionally substituted alkenyl, optionally substituted alkynyl, optionally substituted cycloalkyl, optionally substituted amino, optionally substituted heterocycloalkyl, optionally substituted aryl, optionally substituted heteroaryl, —OR¹⁵, —SR¹⁵, —S(O)R¹⁶, —S(O)₂R¹⁶, —S(O)₂NR¹⁰R¹¹, —NR¹⁰R¹¹, —NR¹¹C(O)NR¹⁰R¹¹, —NR¹¹C(S)NR¹⁰R¹¹, —NR¹¹S(O)₂R¹⁴, —NR¹¹C(O)OR¹³, —NR¹¹C(O)R¹², —C(NR¹¹)NR¹⁰R¹¹, —C(O)NR¹⁰R¹¹, —C(O)OR¹³, —CN, —NO₂, and —C(O)R¹²;

R² is selected from halogen, optionally substituted alkyl, optionally substituted alkenyl, optionally substituted alkynyl, optionally substituted cycloalkyl, optionally substituted amino, optionally substituted heterocycloalkyl, optionally substituted aryl, optionally substituted heteroaryl, —OR¹⁵, —SR¹⁵, —S(O)R¹⁶, —S(O)₂R¹⁶, —S(O)₂NR¹⁰R¹¹, —NR¹⁰R¹¹, —NR¹¹C(O)NR¹⁰R¹¹, —NR¹¹C(S)NR¹⁰R¹¹, —NR¹¹S(O)₂R¹⁴—NR¹¹C(O)OR¹³, —NR¹¹C(O)R¹², —C(NR¹¹)NR¹⁰R¹¹, —C(O)NR¹⁰R¹¹, —C(O)OR¹³, —CN, —NO₂, and —C(O)R¹²;

R³ is absent or is selected from hydrogen, halogen, optionally substituted alkyl, optionally substituted alkenyl, optionally substituted alkynyl, optionally substituted cycloalkyl, optionally substituted amino, optionally substituted heterocycloalkyl, optionally substituted aryl, optionally substituted heteroaryl, —OR¹⁵, —SR¹⁵, —S(O)R¹⁶, —S(O)₂R¹⁶, —S(O)₂NR¹⁰R¹¹, —NR¹⁰R¹¹, —NR¹¹C(O)NR¹⁰R¹¹, —NR¹¹C(S)NR¹⁰R¹¹, —NR¹¹S(O)₂R¹⁴—NR¹¹C(O)OR¹³, —NR¹¹C(O)R¹², —C(NR¹¹)NR¹⁰R¹¹, —C(O)NR¹⁰R¹¹, —C(O)OR¹³, —CN, —NO₂, and —C(O)R¹²;

R⁴ is selected from hydrogen, halogen, optionally substituted alkyl, optionally substituted alkenyl, optionally substituted alkynyl, optionally substituted cycloalkyl, optionally substituted amino, optionally substituted heterocycloalkyl, optionally substituted aryl, optionally substituted heteroaryl, —OR¹⁵, —SR¹⁵, —S(O)R¹⁶, —S(O)₂R¹⁶, —S(O)₂NR¹⁰R¹¹, —NR¹⁰R¹¹, —NR¹¹C(O)NR¹⁰R¹¹, —NR¹¹C(S)NR¹⁰R¹¹, —NR¹¹S(O)₂R₁₄—NR¹¹C(O)OR¹³, —NR¹¹C(O)R¹², —C(NR¹¹)NR¹⁰R¹¹, —C(O)NR¹⁰R¹¹, —C(O)OR¹³, —CN, —NO₂, and —C(O)R¹²;

R⁵ is selected from halogen, optionally substituted alkyl, optionally substituted alkenyl, optionally substituted alkynyl, optionally substituted cycloalkyl, optionally substituted amino, optionally substituted heterocycloalkyl, optionally substituted aryl, optionally substituted heteroaryl, —OR¹⁵, —SR¹⁵, —S(O)R¹⁶, —S(O)₂R⁶, —S(O)₂NR¹⁰R¹¹, —NR¹⁰R¹¹, —NR¹¹C(O)NR¹⁰R¹¹, —NR¹¹C(S)NR¹⁰R¹¹, —NR¹¹S(O)₂R¹⁴—NR¹¹C(O)OR¹³, —NR¹¹C(O)R¹², —C(NR¹¹)NR¹⁰R¹¹, —C(O)NR¹⁰R¹¹, —C(O)OR¹³, —CN, —NO₂, and —C(O)R¹²;

R⁶ is selected from hydrogen, halogen, optionally substituted alkyl, optionally substituted alkenyl, optionally substituted alkynyl, optionally substituted cycloalkyl, optionally substituted amino, optionally substituted heterocycloalkyl, optionally substituted aryl, optionally substituted heteroaryl, —OR¹⁵, —SR¹⁵, —S(O)R⁶, —S(O)₂R¹⁶, —S(O)₂NR¹⁰R¹¹, —NR¹⁰R¹¹, —NR¹¹C(O)NR¹⁰R¹¹, —NR¹¹C(S)NR¹⁰R¹¹, —NR¹¹S(O)₂R¹⁴—NR¹¹C(O)OR³, —NR¹¹C(O)R¹², —C(NR¹¹)NR¹⁰R¹¹, —C(O)NR¹⁰R¹¹, —C(O)OR³, —CN, —NO₂, and —C(O)R¹²;

R⁷ is selected from halogen, optionally substituted alkyl, optionally substituted alkenyl, optionally substituted alkynyl, optionally substituted cycloalkyl, optionally substituted amino, optionally substituted heterocycloalkyl, optionally substituted aryl, optionally substituted heteroaryl, —OR¹⁵, —SR¹⁵, —S(O)R¹⁶, —S(O)₂R¹⁶, —S(O)₂NR¹⁰R¹¹, —NR¹⁰R¹¹, —NR¹¹C(O)NR¹⁰R¹¹, —NR¹¹C(S)NR¹⁰R¹¹, —NR¹¹S(O)₂R¹⁴—NR¹¹C(O)OR¹³, —NR¹¹C(O)R¹², —C(NR¹¹)NR¹⁰R¹¹, —C(O)NR¹⁰R¹¹, —C(O)OR¹³, —CN, —NO₂, and —C(O)R¹²;

R¹⁰ and R¹¹ are independently selected from hydrogen, optionally substituted alkyl, optionally substituted amino, optionally substituted alkoxy, optionally substituted cycloalkyl, optionally substituted heterocycloalkyl, optionally substituted aryl, and optionally substituted heteroaryl, or R¹⁰ and R¹¹, taken together with any intervening atoms, form a ring system selected from optionally substituted heterocycloalkyl, and optionally substituted heteroaryl;

R¹² is selected from hydrogen, optionally substituted alkyl, optionally substituted cycloalkyl, optionally substituted heterocycloalkyl, optionally substituted aryl, and optionally substituted heteroaryl;

R¹³ is selected from hydrogen, optionally substituted alkyl, optionally substituted cycloalkyl, optionally substituted heterocycloalkyl, optionally substituted aryl, and optionally substituted heteroaryl;

R¹⁴ is selected from optionally substituted alkyl, optionally substituted cycloalkyl, optionally substituted heterocycloalkyl, optionally substituted aryl, and optionally substituted heteroaryl;

R¹⁵ is selected from hydrogen, optionally substituted alkyl, optionally substituted cycloalkyl, optionally substituted heterocycloalkyl, optionally substituted aryl, and optionally substituted heteroaryl; and

R¹⁶ is selected from optionally substituted alkyl, optionally substituted cycloalkyl, optionally substituted heterocycloalkyl, optionally substituted aryl, and optionally substituted heteroaryl;

provided that
if W¹ is NR¹ and W³ is NR³, then R³ is absent;
if W³ is NR³ and W¹ is NR¹, then R¹ is absent;
at least one of W¹, W³, W⁸, and W⁹ is N;
no more than four of W¹, W³, W⁴, W⁶, W⁸, and W⁹ are N; and
if W¹ is N, W⁴ is N, and W⁶ is CR⁶, then W⁸ is not N;

and further provided that the compound of Formula 1 is not
(5-(5-chlorothiophen-2-yl)-7-(trifluoromethyl)pyrazolo[1,5-a]pyridin-2-yl)(3-(3,4-dimethoxyphenyl)-5-(2-hydroxyphenyl)-4,5-dihydro-1H-pyrazol-1-yl)methanone;
(5-(5-chlorothiophen-2-yl)-7-(trifluoromethyl)pyrazolo[1,5-a]pyridin-2-yl)(3-(2,5-dimethylphenyl)-5-(2-hydroxyphenyl)-4,5-dihydro-1H-pyrazol-1-yl)methanone; or
(5-(5-chlorothiophen-2-yl)-7-(trifluoromethyl)pyrazolo[1,5-a]pyridin-2-yl)(3-(3,4-dichlorophenyl)-5-(2-hydroxyphenyl)-4,5-dihydro-1H-pyrazol-1-yl)methanone.

Also provided is a pharmaceutical composition comprising a pharmaceutically acceptable diluent and a therapeutically effective amount of at least one chemical entity described herein.

Also provided is a pharmaceutical composition comprising a pharmaceutically acceptable diluent and a therapeutically effective amount of at least one chemical entity chosen from compounds of Formula 1a

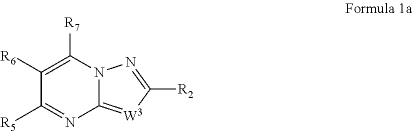

and pharmaceutically acceptable salts thereof, wherein

W³ is selected from CR³ and NR³;

R² is selected from halogen, optionally substituted alkyl, optionally substituted alkenyl, optionally substituted alkynyl, optionally substituted cycloalkyl, optionally substituted amino, optionally substituted heterocycloalkyl, optionally substituted aryl, optionally substituted heteroaryl, —OR¹⁵, —SR¹⁵, —S(O)R¹⁶, —S(O)₂R¹⁶, —S(O)₂NR¹⁰R¹¹, —NR¹⁰R¹¹, —NR¹¹C(O)NR¹⁰R¹¹, —NR¹¹C(S)NR¹⁰R¹¹, —NR¹¹S(O)₂R¹⁴—NR¹¹C(O)OR¹³, —NR¹¹C(O)R¹², —C(NR¹¹)NR¹⁰R¹¹, —C(O)NR¹⁰R¹¹, —C(O)OR¹³, —CN, —NO₂, and —C(O)R¹²;

R³ is absent or is selected from halogen, optionally substituted alkenyl, optionally substituted alkynyl, optionally substituted cycloalkyl, optionally substituted amino, optionally substituted heterocycloalkyl, optionally substituted aryl, optionally substituted heteroaryl, —OR⁵, —SR¹⁵, —S(O)R¹⁶, —S(O)₂R¹⁶, —S(O)₂NR¹⁰R¹¹, —NR¹⁰R¹¹, —NR¹¹C(O)NR¹⁰R¹¹, —NR¹¹C(S)NR¹⁰R¹¹, —NR¹¹S(O)₂R¹⁴—NR¹¹C(O)OR¹³, —NR¹¹C(O)R¹², —C(NR¹¹)NR¹⁰R¹¹, —C(O)NR¹⁰R¹¹, —C(O)OR¹³, —CN, —NO₂, and —C(O)R¹²;

R⁵ is selected from halogen, optionally substituted alkyl, optionally substituted alkenyl, optionally substituted alkynyl, optionally substituted cycloalkyl, optionally substituted amino, optionally substituted heterocycloalkyl, optionally substituted aryl, optionally substituted heteroaryl, —OR¹⁵, —SR¹⁵, —S(O)R¹⁶, —S(O)₂R¹⁶, —S(O)₂NR¹⁰R¹¹, —NR¹⁰R¹¹, —NR¹¹C(O)NR¹⁰R¹¹, —NR¹¹C(S)NR¹⁰R¹¹, —NR¹¹S(O)₂R¹⁴, —NR¹¹C(O)OR¹³, —NR¹¹C(O)R¹², —C(NR¹¹)NR¹⁰R¹¹, —C(O)NR¹⁰R¹¹, —C(O)OR¹³, —CN, —NO₂, and —C(O)R¹²;

R⁶ is selected from hydrogen, halogen, optionally substituted alkyl, optionally substituted alkenyl, optionally substituted alkynyl, optionally substituted cycloalkyl, optionally substituted amino, optionally substituted heterocycloalkyl, optionally substituted aryl, optionally substituted heteroaryl, —OR¹⁵, —SR¹⁵, —S(O)R¹⁶, —S(O)₂R¹⁶, —S(O)₂NR¹⁰R¹¹, —NR¹⁰R¹¹, —NR¹¹C(O)NR¹⁰R¹¹, —NR¹¹C(S)NR¹⁰R¹¹, —NR¹¹S(O)₂R¹⁴—NR¹¹C(O)OR¹³, —NR¹¹C(O)R¹², —C(NR¹¹)NR¹⁰R¹¹, —C(O)NR¹⁰R¹¹, —C(O)OR¹³, —CN, —NO₂, and —C(O)R¹²;

R⁷ is selected from halogen, optionally substituted alkyl, optionally substituted alkenyl, optionally substituted alkynyl, optionally substituted cycloalkyl, optionally substituted amino, optionally substituted heterocycloalkyl, optionally substituted aryl, optionally substituted heteroaryl, —OR¹⁵, —SR¹⁵, —S(O)R¹⁶, —S(O)₂R¹⁶, —S(O)₂NR¹⁰R¹¹, —NR¹⁰R¹¹, —NR¹¹C(O)NR¹⁰R¹¹, —NR¹¹C(S)NR¹⁰R¹¹, —NR¹¹S(O)₂R¹⁴—NR¹¹C(O)OR¹³, —NR¹¹C(O)R¹², —C(NR¹¹)NR¹⁰R¹¹, —C(O)NR¹⁰R¹¹, —C(O)OR¹³, —CN, —NO₂, and —C(O)R¹²;

R¹⁰ and R¹¹ are independently selected from hydrogen, optionally substituted alkyl, optionally substituted amino, optionally substituted alkoxy, optionally substituted cycloalkyl, optionally substituted heterocycloalkyl, optionally substituted aryl, and optionally substituted heteroaryl, or R¹⁰ and R¹¹, taken together with any intervening atoms, form a ring system selected from optionally substituted heterocycloalkyl, and optionally substituted heteroaryl;

R¹² is selected from hydrogen, optionally substituted alkyl, optionally substituted cycloalkyl, optionally substituted heterocycloalkyl, optionally substituted aryl, and optionally substituted heteroaryl;

R¹³ is selected from hydrogen, optionally substituted alkyl, optionally substituted cycloalkyl, optionally substituted heterocycloalkyl, optionally substituted aryl, and optionally substituted heteroaryl;

R¹⁴ is selected from optionally substituted alkyl, optionally substituted cycloalkyl, optionally substituted heterocycloalkyl, optionally substituted aryl, and optionally substituted heteroaryl;

R¹⁵ is selected from hydrogen, optionally substituted alkyl, optionally substituted cycloalkyl, optionally substituted heterocycloalkyl, optionally substituted aryl, and optionally substituted heteroaryl; and

R¹⁶ is selected from optionally substituted alkyl, optionally substituted cycloalkyl, optionally substituted heterocycloalkyl, optionally substituted aryl, and optionally substituted heteroaryl.

Also provided is a pharmaceutical composition comprising a pharmaceutically acceptable diluent and a therapeutically effective amount of at least one chemical entity chosen from
(5-(5-chlorothiophen-2-yl)-7-(trifluoromethyl)pyrazolo[1,5-a]pyridin-2-yl)(3-(3,4-dimethoxyphenyl)-5-(2-hydroxyphenyl)-4,5-dihydro-1H-pyrazol-1-yl)methanone;
(5-(5-chlorothiophen-2-yl)-7-(trifluoromethyl)pyrazolo[1,5-a]pyridin-2-yl)(3-(2,5-dimethylphenyl)-5-(2-hydroxyphenyl)-4,5-dihydro-1H-pyrazol-1-yl)methanone; or
(5-(5-chlorothiophen-2-yl)-7-(trifluoromethyl)pyrazolo[1,5-a]pyridin-2-yl)(3-(3,4-dichlorophenyl)-5-(2-hydroxyphenyl)-4,5-dihydro-1H-pyrazol-1-yl)methanone,
   and pharmaceutically acceptable salts thereof.

Also provided are methods for treating a viral infection mediated at least in part by a virus in the flaviviridae family of viruses, such as HCV, in mammals which methods comprise administering to a mammal, that has been diagnosed with said viral infection or is at risk of developing said viral infection, a pharmaceutical composition described herein.

Other aspects and embodiments will be apparent to those skilled in the art from the following detailed description.

As used in the present specification, the following words and phrases are generally intended to have the meanings as set forth below, except to the extent that the context in which they are used indicates otherwise.

The following abbreviations and terms have the indicated meanings throughout:

HCV: hepacivirus
HIV: human immunodeficiency virus
IFN: interferon
IMPDH: inosine 5′-monophosphate dehydrogenase
mg: milligram
kg: kilogram
MDI: metered dose inhaler
DPI: dry powder inhaler
nM: nano-Molar
wt %: weight percent
M: micro-Molar
EC₅₀: effective concentration of compound at 50% inhibition is observed
TC₅₀: toxic concentration of compound at which 50% inhibition is observed
b: Hill's coefficient
g: gram

### K: Kelvin

mL: milli-Liter
1N: 1 Normal concentration
AIDS: Acquired Immunodeficiency syndrome

It is to be understood that the terminology used herein is for the purpose of describing particular embodiments only and is not intended to limit the scope of the present specification. In this specification and in the claims that follow, reference will be made to a number of terms that shall be defined to have the following meanings:

“Alkyl” refers to monovalent saturated aliphatic hydrocarbyl groups having from 1 to carbon atoms and, in some embodiments, from 1 to 6 carbon atoms. “C_{x-y}alkyl” refers to alkyl groups having from x to y carbon atoms. This term includes, by way of example, linear and branched hydrocarbyl groups such as methyl (CH₃—), ethyl (CH₃CH₂—), n-propyl (CH₃CH₂CH₂—), isopropyl ((CH₃)₂CH—), n-butyl (CH₃CH₂CH₂CH₂—), isobutyl ((CH₃)₂CHCH₂—), sec-butyl ((CH₃)(CH₃CH₂)CH—), t-butyl ((CH₃)₃C—), n-pentyl (CH₃CH₂CH₂CH₂CH₂—), and neopentyl ((CH₃)₃CCH₂—).

“Substituted alkyl” refers to an alkyl group having from 1 to 5 and, in some embodiments, 1 to 3 or 1 or 2 substituents selected from alkenyl, substituted alkenyl, alkynyl, substituted alkynyl, alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, azido, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, guanidino, substituted guanidino, halo, hydroxy, hydroxyamino, alkoxyamino, hydrazino, substituted hydrazino, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, spirocycloalkyl, SO₃H, substituted sulfonyl, sulfonyloxy, thioacyl, thiocyanate, thiol, alkylthio, and substituted alkylthio, wherein said substituents are as defined herein.

“Alkylidene” or “alkylene” refers to divalent saturated aliphatic hydrocarbyl groups having from 1 to 10 carbon atoms and, in some embodiments, from 1 to 6 carbon atoms. “(Cᵤ₋ᵥ)alkylene” refers to alkylene groups having from u to v carbon atoms. The alkylidene and alkylene groups include branched and straight chain hydrocarbyl groups. For example “(C₁₋₆)alkylene” is meant to include methylene, ethylene, propylene, 2-methypropylene, pentylene, and the like.

“Substituted alkylidene” or “substituted alkylene” refers to an alkylidene group having from 1 to 5 and, in some embodiments, 1 to 3 or 1 or 2 substituents selected from alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, azido, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, guanidino, substituted guanidino, halo, hydroxy, hydroxyamino, alkoxyamino, hydrazino, substituted hydrazino, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, oxo, thione, spirocycloalkyl, SO₃H, substituted sulfonyl, sulfonyloxy, thioacyl, thiocyanate, thiol, alkylthio, and substituted alkylthio, wherein said substituents are as defined herein.

“Alkenyl” refers to a linear or branched hydrocarbyl group having from 2 to 10 carbon atoms and in some embodiments from 2 to 6 carbon atoms or 2 to 4 carbon atoms and having at least 1 site of vinyl unsaturation (>C═C<). For example, (Cₓ-C_{y})alkenyl refers to alkenyl groups having from x to y carbon atoms and is meant to include for example, ethenyl, propenyl, 1,3-butadienyl, and the like.

“Substituted alkenyl” refers to alkenyl groups having from 1 to 3 substituents and, in some embodiments, 1 or 2 substituents selected from alkoxy, substituted alkoxy, acyl, acylamino, acyloxy, alkyl, substituted alkyl, alkynyl, substituted alkynyl, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, guanidino, substituted guanidino, halo, hydroxy, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, sulfonyloxy, thioacyl, thiol, alkylthio, and substituted alkylthio, wherein said substituents are defined herein and with the proviso that any hydroxy or thiol substitution is not attached to a vinyl (unsaturated) carbon atom.

“Alkynyl” refers to a linear monovalent hydrocarbon radical or a branched monovalent hydrocarbon radical containing at least one triple bond. The term “alkynyl” is also meant to include those hydrocarbyl groups having one triple bond and one double bond. For example, (C₂-C₆)alkynyl is meant to include ethynyl, propynyl, and the like.

“Substituted alkynyl” refers to alkynyl groups having from 1 to 3 substituents and, in some embodiments, from 1 or 2 substituents selected from alkoxy, substituted alkoxy, acyl, acylamino, acyloxy, alkyl, substituted alkyl, alkenyl, substituted alkenyl, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, guanidino, substituted guanidino, halo, hydroxy, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, sulfonyloxy, thioacyl, thiol, alkylthio, and substituted alkylthio, wherein said substituents are as defined herein and with the proviso that any hydroxy or thiol substitution is not attached to an acetylenic carbon atom.

“Alkoxy” refers to the group —O-alkyl wherein alkyl is defined herein. Alkoxy includes, by way of example, methoxy, ethoxy, n-propoxy, isopropoxy, n-butoxy, t-butoxy, sec-butoxy, and n-pentoxy.

“Substituted alkoxy” refers to the group —O-(substituted alkyl) wherein substituted alkyl is as defined herein.

“Acyl” refers to the groups H—C(O)—, alkyl-C(O)—, substituted alkyl-C(O)—, alkenyl-C(O)—, substituted alkenyl-C(O)—, alkynyl-C(O)—, substituted alkynyl-C(O)—, cycloalkyl-C(O)—, substituted cycloalkyl-C(O)—, aryl-C(O)—, substituted aryl-C(O)—, substituted hydrazino-C(O)—, heteroaryl-C(O)—, substituted heteroaryl-C(O)—, heterocyclic-C(O)—, and substituted heterocyclic-C(O)—, wherein alkyl, substituted alkyl, alkenyl, substituted alkenyl, alkynyl, substituted alkynyl, cycloalkyl, substituted cycloalkyl, aryl, substituted aryl, substituted hydrazino, heteroaryl, substituted heteroaryl, heterocyclic, and substituted heterocyclic are as defined herein. Acyl includes the “acetyl” group CH₃C(O)—.

“Acylamino” refers to the groups —NR²⁰C(O)alkyl, —NR²⁰C(O)substituted alkyl, —NR²⁰C(O)cycloalkyl, —NR²⁰C(O)substituted cycloalkyl, —NR²⁰C(O)alkenyl, —NR²⁰C(O)substituted alkenyl, —NR²⁰C(O)alkynyl, —NR²⁰C(O)substituted alkynyl, —NR²⁰C(O)aryl, —NR²⁰C(O)substituted aryl, —NR²⁰C(O)heteroaryl, —NR²⁰C(O)substituted heteroaryl, —NR²⁰C(O)heterocyclic, and —NR²⁰C(O)substituted heterocyclic wherein R²⁰ is hydrogen or alkyl and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein.

“Acyloxy” refers to the groups alkyl-C(O)O—, substituted alkyl-C(O)O—, alkenyl-C(O)O—, substituted alkenyl-C(O)O—, alkynyl-C(O)O—, substituted alkynyl-C(O)O—, aryl-C(O)O—, substituted aryl-C(O)O—, cycloalkyl-C(O)O—, substituted cycloalkyl-C(O)O—, heteroaryl-C(O)O—, substituted heteroaryl-C(O)O—, heterocyclic-C(O)O—, and substituted heterocyclic-C(O)O— wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein.

“Amino” refers to the group —NH₂.

“Substituted amino” refers to the group —NR²¹R²² where R²¹ and R²² are independently selected from hydrogen, alkyl, substituted alkyl, alkenyl, substituted alkenyl, alkynyl, substituted alkynyl, aryl, substituted aryl, cycloalkyl, substituted cycloalkyl, heteroaryl, substituted heteroaryl, heterocyclic, substituted heterocyclic, —SO₂-alkyl, —SO₂-substituted alkyl, —SO₂-alkenyl, —SO₂-substituted alkenyl, —SO₂-cycloalkyl, —SO₂-substituted cylcoalkyl, —SO₂-aryl, —SO₂-substituted aryl, —SO₂-heteroaryl, —SO₂-substituted heteroaryl, —SO₂-heterocyclic, and —SO₂-substituted heterocyclic and wherein R²¹ and R²² are optionally joined together with the nitrogen bound thereto to form a heterocyclic or substituted heterocyclic group, provided that R²¹ and R²² are both not hydrogen, and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein. When R²¹ is hydrogen and R²² is alkyl, the substituted amino group is sometimes referred to herein as alkylamino. When R²¹ and R²² are alkyl, the substituted amino group is sometimes referred to herein as dialkylamino. When referring to a monosubstituted amino, it is meant that either R²¹ or R²² is hydrogen but not both. When referring to a disubstituted amino, it is meant that neither R²¹ nor R²² are hydrogen.

“Hydroxyamino” refers to the group —NHOH.

“Alkoxyamino” refers to the group —NHO-alkyl wherein alkyl is defined herein.

“Aminocarbonyl” refers to the group —C(O)NR²³R²⁴ where R²³ and R²⁴ are independently selected from hydrogen, alkyl, substituted alkyl, alkenyl, substituted alkenyl, alkynyl, substituted alkynyl, aryl, substituted aryl, cycloalkyl, substituted cycloalkyl, heteroaryl, substituted heteroaryl, heterocyclic, substituted heterocyclic, hydroxy, alkoxy, substituted alkoxy, amino, substituted amino, and acylamino, and where R²³ and R²⁴ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein.

“Aminothiocarbonyl” refers to the group —C(S)NR²³R²⁴ where R²³ and R²⁴ are independently selected from hydrogen, alkyl, substituted alkyl, alkenyl, substituted alkenyl, alkynyl, substituted alkynyl, aryl, substituted aryl, cycloalkyl, substituted cycloalkyl, heteroaryl, substituted heteroaryl, heterocyclic, and substituted heterocyclic and where R²³ and R²⁴ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein.

“Aminocarbonylamino” refers to the group —NR²⁰C(O)NR²³R²⁴ where R²⁰ is hydrogen or alkyl and R²³ and R²⁴ are independently selected from hydrogen, alkyl, substituted alkyl, alkenyl, substituted alkenyl, alkynyl, substituted alkynyl, aryl, substituted aryl, cycloalkyl, substituted cycloalkyl, heteroaryl, substituted heteroaryl, heterocyclic, and substituted heterocyclic and where R²³ and R²⁴ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein.

“Aminothiocarbonylamino” refers to the group —NR²⁰C(S)NR²³R²⁴ where R²⁰ is hydrogen or alkyl and R²³ and R²⁴ are independently selected from hydrogen, alkyl, substituted alkyl, alkenyl, substituted alkenyl, alkynyl, substituted alkynyl, aryl, substituted aryl, cycloalkyl, substituted cycloalkyl, heteroaryl, substituted heteroaryl, heterocyclic, and substituted heterocyclic and where R²³ and R²⁴ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein.

“Aminocarbonyloxy” refers to the group —O—C(O)NR²³R²⁴ where R²³ and R²⁴ are independently selected from hydrogen, alkyl, substituted alkyl, alkenyl, substituted alkenyl, alkynyl, substituted alkynyl, aryl, substituted aryl, cycloalkyl, substituted cycloalkyl, heteroaryl, substituted heteroaryl, heterocyclic, and substituted heterocyclic and where R²³ and R²⁴ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein.

“Aminosulfonyl” refers to the group —SO₂NR²³R²⁴ where R²³ and R²⁴ are independently selected from hydrogen, alkyl, substituted alkyl, alkenyl, substituted alkenyl, alkynyl, substituted alkynyl, aryl, substituted aryl, cycloalkyl, substituted cycloalkyl, heteroaryl, substituted heteroaryl, heterocyclic, and substituted heterocyclic and where R²³ and R²⁴ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein.

“Aminosulfonyloxy” refers to the group —O—SO₂NR²³R²⁴ where R²³ and R²⁴ are independently selected from hydrogen, alkyl, substituted alkyl, alkenyl, substituted alkenyl, alkynyl, substituted alkynyl, aryl, substituted aryl, cycloalkyl, substituted cycloalkyl, heteroaryl, substituted heteroaryl, heterocyclic, and substituted heterocyclic and where R²³ and R²⁴ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein.

“Aminosulfonylamino” refers to the group —NR²⁰—SO₂NR²³R²⁴ where R²⁰ is hydrogen or alkyl and R²³ and R²⁴ are independently selected from hydrogen, alkyl, substituted alkyl, alkenyl, substituted alkenyl, alkynyl, substituted alkynyl, aryl, substituted aryl, cycloalkyl, substituted cycloalkyl, heteroaryl, substituted heteroaryl, heterocyclic, and substituted heterocyclic and where R²³ and R²⁴ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein.

“Amidino” refers to the group —C(═NR²⁵)NR²³R²⁴ where R²⁵, R²³, and R²⁴ are independently selected from hydrogen, alkyl, substituted alkyl, alkenyl, substituted alkenyl, alkynyl, substituted alkynyl, aryl, substituted aryl, cycloalkyl, substituted cycloalkyl, heteroaryl, substituted heteroaryl, heterocyclic, and substituted heterocyclic and where R²³ and R²⁴ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein.

“Aryl” or “Ar” refers to an aromatic group of from 6 to 14 carbon atoms and no ring heteroatoms and having a single ring (e.g., phenyl) or multiple condensed (fused) rings (e.g., naphthyl or anthryl). For multiple ring systems, including fused, bridged, and spiro ring systems having aromatic and non-aromatic rings that have no ring heteroatoms, the term “Aryl” or “Ar” applies when the point of attachment is at an aromatic carbon atom (e.g., 5,6,7,8 tetrahydronaphthalene-2-Y1 is an aryl group as its point of attachment is at the 2-position of the aromatic phenyl ring).

“Substituted aryl” refers to aryl groups which are substituted with 1 to 8 and, in some embodiments, 1 to 5, 1 to 3, or 1 or 2 substituents selected from alkyl, substituted alkyl, alkenyl, substituted alkenyl, alkynyl, substituted alkynyl, alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, azido, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, guanidino, substituted guanidino, halo, hydroxy, hydroxyamino, alkoxyamino, hydrazino, substituted hydrazino, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, sulfonyloxy, thioacyl, thiocyanate, thiol, alkylthio, and substituted alkylthio, wherein said substituents are defined herein.

“Aryloxy” refers to the group —O-aryl, where aryl is as defined herein, that includes, by way of example, phenoxy and naphthyloxy.

“Substituted aryloxy” refers to the group —O-(substituted aryl) where substituted aryl is as defined herein.

“Arylthio” refers to the group —S-aryl, where aryl is as defined herein.

“Substituted arylthio” refers to the group —S-(substituted aryl), where substituted aryl is as defined herein.

“Azido” refers to the group —N₃.

“Hydrazino” refers to the group —NHNH₂.

“Substituted hydrazino” refers to the group —NR²⁶NR²⁷R²⁸ where R²⁶, R²⁷, and R²⁸ are independently selected from hydrogen, alkyl, substituted alkyl, alkenyl, substituted alkenyl, alkynyl, substituted alkynyl, aryl, substituted aryl, carboxyl ester, cycloalkyl, substituted cycloalkyl, heteroaryl, substituted heteroaryl, heterocyclic, substituted heterocyclic, —SO₂-alkyl, —SO₂-substituted alkyl, —SO₂-alkenyl, —SO₂-substituted alkenyl, —SO₂-cycloalkyl, —SO₂-substituted cylcoalkyl, —SO₂-aryl, —SO₂-substituted aryl, —SO₂-heteroaryl, —SO₂-substituted heteroaryl, —SO₂-heterocyclic, and —SO₂-substituted heterocyclic and wherein R²⁷ and R²⁸ are optionally joined, together with the nitrogen bound thereto to form a heterocyclic or substituted heterocyclic group, provided that R²⁷ and R²⁸ are both not hydrogen, and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein.

“Cyano” or “carbonitrile” refers to the group —CN.

“Carbonyl” refers to the divalent group —C(O)— which is equivalent to —C(═O)—.

“Carboxyl” or “carboxy” refers to —COOH or salts thereof.

“Carboxyl ester” or “carboxy ester” refers to the groups —C(O)O-alkyl, —C(O)O-substituted alkyl, —C(O)O-alkenyl, —C(O)O-substituted alkenyl, —C(O)O-alkynyl, —C(O)O-substituted alkynyl, —C(O)O-aryl, —C(O)O-substituted aryl, —C(O)O-cycloalkyl, —C(O)O-substituted cycloalkyl, —C(O)O-heteroaryl, —C(O)O-substituted heteroaryl, —C(O)O-heterocyclic, and —C(O)O-substituted heterocyclic wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein.

“(Carboxyl ester)amino” refers to the group —NR²⁰—C(O)O-alkyl, —NR²⁰—C(O)O-substituted alkyl, —NR²⁰—C(O)O-alkenyl, —NR²⁰—C(O)O-substituted alkenyl, —NR²⁰—C(O)O-alkynyl, —NR²⁰—C(O)O-substituted alkynyl, —NR²⁰—C(O)O-aryl, —NR²⁰—C(O)O-substituted aryl, —NR²⁰—C(O)O-cycloalkyl, —NR²⁰—C(O)O-substituted cycloalkyl, —NR²⁰—C(O)O-heteroaryl, —NR²⁰—C(O)O-substituted heteroaryl, —NR²⁰—C(O)O-heterocyclic, and —NR²⁰—C(O)O-substituted heterocyclic wherein R²⁰ is alkyl or hydrogen, and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein.

“(Carboxyl ester)oxy” refers to the group —O—C(O)O-alkyl, —O—C(O)O-substituted alkyl, —O—C(O)O-alkenyl, —O—C(O)O-substituted alkenyl, —O—C(O)O-alkynyl, —O—C(O)O-substituted alkynyl, —O—C(O)O-aryl, —O—C(O)O-substituted aryl, —O—C(O)O-cycloalkyl, —O—C(O)O-substituted cycloalkyl, —O—C(O)O-heteroaryl, —O—C(O)O-substituted heteroaryl, —O—C(O)O-heterocyclic, and —O—C(O)O-substituted heterocyclic wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein.

“Cycloalkyl” refers to a saturated or partially saturated cyclic group of from 3 to 14 carbon atoms and no ring heteroatoms and having a single ring or multiple rings including fused, bridged, and spiro ring systems. For multiple ring systems having aromatic and non-aromatic rings that have no ring heteroatoms, the term “cycloalkyl” applies when the point of attachment is at a non-aromatic carbon atom (e.g. 5,6,7,8,-tetrahydronaphthalene-5-yl). The term “Cycloalkyl” includes cycloalkenyl groups. Examples of cycloalkyl groups include, for instance, adamantyl, cyclopropyl, cyclobutyl, cyclopentyl, cyclooctyl, and cyclohexenyl. “Cᵤ₋ᵥcycloalkyl” refers to cycloalkyl groups having u to v carbon atoms.

“Cycloalkenyl” refers to a partially saturated cycloalkyl ring having at least one site of >C═C< ring unsaturation.

“Cycloalkylene” refer to divalent cycloalkyl groups as defined herein. Examples of cycloalkyl groups include those having three to six carbon ring atoms such as cyclopropylene, cyclobutylene, cyclopentylene, and cyclohexylene.

“Substituted cycloalkyl” refers to a cycloalkyl group, as defined herein, having from 1 to 8, or 1 to 5, or in some embodiments 1 to 3 substituents selected from oxo, thione, alkyl, substituted alkyl, alkenyl, substituted alkenyl, alkynyl, substituted alkynyl, alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, azido, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, guanidino, substituted guanidino, halo, hydroxy, hydroxyamino, alkoxyamino, hydrazino, substituted hydrazino, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, sulfonyloxy, thioacyl, thiocyanate, thiol, alkylthio, and substituted alkylthio, wherein said substituents are as defined herein. The term “substituted cycloalkyl” includes substituted cycloalkenyl groups.

“Cycloalkyloxy” refers to —O-cycloalkyl wherein cycloalkyl is as defined herein.

“Substituted cycloalkyloxy refers to —O-(substituted cycloalkyl) wherein substituted cycloalkyl is as defined herein.

“Cycloalkylthio” refers to —S-cycloalkyl wherein cycloalkyl is as defined herein.

“Substituted cycloalkylthio” refers to —S-(substituted cycloalkyl).

“Guanidino” refers to the group —NHC(═NH)NH₂.

“Substituted guanidino” refers to —NR²⁹C(═NR²⁹)N(R²⁹)₂ where each R²⁹ is independently selected from hydrogen, alkyl, substituted alkyl, aryl, substituted aryl, heteroaryl, substituted heteroaryl, heterocyclyl, and substituted heterocyclyl and two R²⁹ groups attached to a common guanidino nitrogen atom are optionally joined together with the nitrogen bound thereto to form a heterocyclic or substituted heterocyclic group, provided that at least one R²⁹ is not hydrogen, and wherein said substituents are as defined herein.

“Halo” or “halogen” refers to fluoro, chloro, bromo, and iodo.

“Haloalkyl” refers to substitution of alkyl groups with 1 to 5 or in some embodiments 1 to 3 halo groups.

“Haloalkoxy” refers to substitution of alkoxy groups with 1 to 5 or in some embodiments 1 to 3 halo groups.

“Hydroxy” or “hydroxyl” refers to the group —OH.

“Heteroaryl” refers to an aromatic group of from 1 to 14 carbon atoms and 1 to 6 heteroatoms selected from oxygen, nitrogen, and sulfur and includes single ring (e.g. imidazolyl) and multiple ring systems (e.g. benzimidazol-2-yl and benzimidazol-6-yl). For multiple ring systems, including fused, bridged, and spiro ring systems having aromatic and non-aromatic rings, the term “heteroaryl” applies if there is at least one ring heteroatom and the point of attachment is at an atom of an aromatic ring (e.g. 1,2,3,4-tetrahydroquinolin-6-yl and 5,6,7,8-tetrahydroquinolin-3-yl). In one embodiment, the carbon, nitrogen and/or the sulfur ring atom(s) of the heteroaryl group are optionally oxidized to provide for the C═O, N-oxide (N—O), sulfinyl, or sulfonyl moieties. More specifically the term heteroaryl includes, but is not limited to, pyridyl, furanyl, thienyl, thiazolyl, isothiazolyl, triazolyl, imidazolyl, isoxazolyl, pyrrolyl, pyrazolyl, pyridazinyl, pyrimidinyl, benzofuranyl, tetrahydrobenzofuranyl, isobenzofuranyl, benzothiazolyl, benzoisothiazolyl, benzotriazolyl, indolyl, isoindolyl, benzoxazolyl, quinolyl, tetrahydroquinolinyl, isoquinolyl, quinazolinonyl, benzimidazolyl, benzisoxazolyl, or benzothienyl.

“Substituted heteroaryl” refers to heteroaryl groups that are substituted with from 1 to 8 or in some embodiments 1 to 5, or 1 to 3, or 1 or 2 substituents selected from the substituents defined for substituted aryl.

“Heteroaryloxy” refers to —O-heteroaryl wherein heteroaryl is as defined herein.

“Substituted heteroaryloxy refers to the group —O-(substituted heteroaryl) wherein substituted heteroaryl is as defined herein.

“Heteroarylthio” refers to the group —S-heteroaryl wherein heteroaryl is as defined herein.

“Substituted heteroarylthio” refers to the group —S-(substituted heteroaryl) wherein substituted heteroaryl is as defined herein.

“Aromatic” indicates that each of ring atoms is essentially in the same plane and has a p-orbital perpendicular to the ring plane, and in which (4n+2)π electrons, when n is 0 or a positive integer, are associated with the ring to comply with Huckel's rule. Aromatic ring systems may be depicted as a circle, which represents the (4n+2)π electrons, enclosed by an outer cyclic structure, such as, a hexagon or pentagon. For example, each of the rings in the compound of Formula 1 is aromatic.

“Heterocyclic” or “heterocycle” or “heterocycloalkyl” or “heterocyclyl” refers to a saturated or partially saturated cyclic group having from 1 to 14 carbon atoms and from 1 to 6 heteroatoms selected from nitrogen, sulfur, phosphorus or oxygen and includes single ring and multiple ring systems including fused, bridged, and spiro ring systems. For multiple ring systems having aromatic and/or non-aromatic rings, the terms “heterocyclic”, “heterocycle”, “heterocycloalkyl”, or “heterocyclyl” apply when there is at least one ring heteroatom and the point of attachment is at an atom of a non-aromatic ring (e.g. 1,2,3,4-tetrahydroquinoline-3-yl, 5,6,7,8-tetrahydroquinoline-6-yl, and decahydroquinolin-6-yl). In one embodiment, the nitrogen, phosphorus and/or sulfur atom(s) of the heterocyclic group are optionally oxidized to provide for the N-oxide, phosphinane oxide, sulfinyl, sulfonyl moieties. More specifically the heterocyclyl includes, but is not limited to, tetrahydropyranyl, piperidinyl, N-methylpiperidin-3-yl, piperazinyl, N-methylpyrrolidin-3-yl, 3-pyrrolidinyl, 2-pyrrolidon-1-yl, morpholinyl, and pyrrolidinyl. A prefix indicating the number of carbon atoms (e.g., C₃-C₁₀) refers to the total number of carbon atoms in the portion of the heterocyclyl group exclusive of the number of heteroatoms.

“Substituted heterocyclic” or “Substituted heterocycle” or “substituted heterocycloalkyl” or “substituted heterocyclyl” refers to heterocyclic groups, as defined herein, that are substituted with from 1 to 5 or in some embodiments 1 to 3 of the substituents as defined for substituted cycloalkyl.

“Heterocyclyloxy” refers to the group —O-heterocycyl wherein heterocyclyl is as defined herein.

“Substituted heterocyclyloxy” refers to the group —O-(substituted heterocycyl) wherein substituted heterocyclyl is as defined herein.

“Heterocyclylthio” refers to the group —S-heterocycyl wherein heterocyclyl is as defined herein.

“Substituted heterocyclylthio” refers to the group —S-(substituted heterocycyl) wherein substituted heterocyclyl is as defined herein.

Examples of heterocycle and heteroaryl groups include, but are not limited to, azetidine, pyrrole, imidazole, pyrazole, pyridine, pyrazine, pyrimidine, pyridazine, pyridone, indolizine, isoindole, indole, dihydroindole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, phenoxazine, phenothiazine, imidazolidine, imidazoline, piperidine, piperazine, indoline, phthalimide, 1,2,3,4-tetrahydroisoquinoline, 4,5,6,7-tetrahydrobenzo[b]thiophene, thiazole, thiazolidine, thiophene, benzo[b]thiophene, morpholine, thiomorpholine (also referred to as thiamorpholine), 1,1-dioxothiomorpholine, piperidine, pyrrolidine, and tetrahydrofuran.

“Nitro” refers to the group —NO₂.

“Oxo” refers to the atom (═O).

“Oxide” refers to products resulting from the oxidation of one or more heteroatoms. Examples include N-oxides, sulfoxides, and sulfones.

“Spirocycloalkyl” refers to a 3 to 10 member cyclic substituent formed by replacement of two hydrogen atoms at a common carbon atom with an alkylene group having 2 to 9 carbon atoms, as exemplified by the following structure wherein the methylene group shown here attached to bonds marked with wavy lines is substituted with a spirocycloalkyl group:

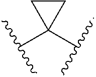

“Sulfonyl” refers to the divalent group —S(O)₂—.

“Substituted sulfonyl” refers to the group —SO₂-alkyl, —SO₂-substituted alkyl, —SO₂-alkenyl, —SO₂-substituted alkenyl, —SO₂-alkynyl, —SO₂-substituted alkynyl, —SO₂-cycloalkyl, —SO₂-substituted cylcoalkyl, —SO₂-aryl, —SO₂-substituted aryl, —SO₂-heteroaryl, —SO₂-substituted heteroaryl, —SO₂-heterocyclic, —SO₂-substituted heterocyclic, wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic and substituted heterocyclic are as defined herein. Substituted sulfonyl includes groups such as methyl-SO₂—, phenyl-SO₂—, and 4-methylphenyl-SO₂—.

“Sulfonyloxy” refers to the group —OSO₂-alkyl, —OSO₂-substituted alkyl, —OSO₂-alkenyl, —OSO₂-substituted alkenyl, —OSO₂-cycloalkyl, —OSO₂-substituted cylcoalkyl, —OSO₂-aryl, —OSO₂-substituted aryl, —OSO₂-heteroaryl, —OSO₂-substituted heteroaryl, —OSO₂-heterocyclic, —OSO₂-substituted heterocyclic, wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic and substituted heterocyclic are as defined herein.

“Thioacyl” refers to the groups H—C(S)—, alkyl-C(S)—, substituted alkyl-C(S)—, alkenyl-C(S)—, substituted alkenyl-C(S)—, alkynyl-C(S)—, substituted alkynyl-C(S)—, cycloalkyl-C(S)—, substituted cycloalkyl-C(S)—, aryl-C(S)—, substituted aryl-C(S)—, heteroaryl-C(S)—, substituted heteroaryl-C(S)—, heterocyclic-C(S)—, and substituted heterocyclic-C(S)—, wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic and substituted heterocyclic are as defined herein.

“Thiol” refers to the group —SH.

“Alkylthio” refers to the group —S-alkyl wherein alkyl is as defined herein.

“Substituted alkylthio” refers to the group —S-(substituted alkyl) wherein substituted alkyl is as defined herein.

“Thiocarbonyl” refers to the divalent group —C(S)— which is equivalent to —C(═S)—.

“Thione” refers to the atom (═S).

“Thiocyanate” refers to the group —SCN.

“Compound” and “compounds” as used herein refers to a compound encompassed by the generic formulae disclosed herein, any subgenus of those generic formulae, and any forms of the compounds within the generic and subgeneric formulae, including the racemates, stereoisomers, and tautomers of the compound or compounds.

“Racemates” refers to a mixture of enantiomers.

“Solvate” or “solvates” of a compound refer to those compounds, where compounds is as defined above, that are bound to a stoichiometric or non-stoichiometric amount of a solvent. Solvates of a compound includes solvates of all forms of the compound. In certain embodiments, solvents are volatile, non-toxic, and/or acceptable for administration to humans in trace amounts. Suitable solvates include water.

“Stereoisomer” or “stereoisomers” refer to compounds that differ in the chirality of one or more stereocenters. Stereoisomers include enantiomers and diastereomers.

“Tautomer” refer to alternate forms of a compound that differ in the position of a proton, such as enol-keto and imine-enamine tautomers, or the tautomeric forms of heteroaryl groups containing a ring atom attached to both a ring —NH— moiety and a ring ═N— moiety such as pyrazoles, imidazoles, benzimidazoles, triazoles, and tetrazoles.

“Pharmaceutically acceptable salt” refers to pharmaceutically acceptable salts derived from a variety of organic and inorganic counter ions well known in the art and include, by way of example only, sodium, potassium, calcium, magnesium, ammonium, and tetraalkylammonium, and when the molecule contains a basic functionality, salts of organic or inorganic acids, such as hydrochloride, hydrobromide, tartrate, mesylate, acetate, maleate, and oxalate. Suitable salts include those described in P. Heinrich Stahl, Camille G. Wermuth (Eds.), Handbook of Pharmaceutical Salts Properties, Selection, and Use; 2002.

“Patient” refers to mammals and includes humans and non-human mammals.

“Treating” or “treatment” of a disease in a patient refers to 1) preventing the disease from occurring in a patient that is predisposed or does not yet display symptoms of the disease; 2) inhibiting the disease or arresting its development; or 3) ameliorating or causing regression of the disease.

Unless indicated otherwise, the nomenclature of substituents that are not explicitly defined herein are arrived at by naming the terminal portion of the functionality followed by the adjacent functionality toward the point of attachment. For example, the substituent “arylalkyloxycabonyl” refers to the group (aryl)-(alkyl)-O—C(O)—.

It is understood that in all substituted groups defined above, polymers arrived at by defining substituents with further substituents to themselves (e.g., substituted aryl having a substituted aryl group as a substituent which is itself substituted with a substituted aryl group, which is further substituted by a substituted aryl group etc.) are not intended for inclusion herein. In such cases, the maximum number of such substitutions is three. For example, serial substitutions of substituted aryl groups with two other substituted aryl groups are limited to -substituted aryl-(substituted aryl)-substituted aryl.

Similarly, it is understood that the above definitions are not intended to include impermissible substitution patterns (e.g., methyl substituted with 5 fluoro groups). Such impermissible substitution patterns are well known to the skilled artisan.

Provided is at least one chemical entity selected from compounds of Formula 1:

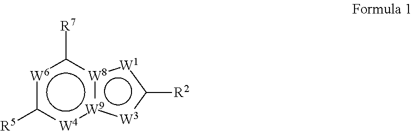

and pharmaceutically acceptable salts thereof, wherein

W¹ is selected from CR¹ and NR¹;

W³ is selected from CR³ and NR³;

W⁴ is selected from CR⁴ and N;

W⁶ is selected from CR⁶ and N;

W⁸ is selected from C and N;

W⁹ is selected from C and N;

R¹ is absent or is selected from hydrogen, halogen, optionally substituted alkyl, optionally substituted alkenyl, optionally substituted alkynyl, optionally substituted cycloalkyl, optionally substituted amino, optionally substituted heterocycloalkyl, optionally substituted aryl, optionally substituted heteroaryl, —OR¹⁵, —SR¹⁵, —S(O)R¹⁶, —S(O)₂R¹⁶, —S(O)₂NR¹⁰R¹¹, —NR¹⁰R¹¹, —NR¹¹C(O)NR¹⁰R¹¹, —NR¹¹C(S)NR¹⁰R¹¹, —NR¹¹S(O)₂R¹⁴—NR¹¹C(O)OR¹³, —NR¹¹C(O)R¹², —C(NR¹¹)NR¹⁰R¹¹, —C(O)NR¹⁰R¹¹, —C(O)OR¹³, —CN, —NO₂, and —C(O)R¹²;

R² is selected from halogen, optionally substituted alkyl, optionally substituted alkenyl, optionally substituted alkynyl, optionally substituted cycloalkyl, optionally substituted amino, optionally substituted heterocycloalkyl, optionally substituted aryl, optionally substituted heteroaryl, —OR¹⁵, —SR¹⁵, —S(O)R¹⁶, —S(O)₂R¹⁶, —S(O)₂NR¹⁰R¹¹, —NR¹⁰R¹¹, —NR¹¹C(O)NR¹⁰R¹¹, —NR¹¹C(S)NR¹⁰R¹¹, —NR¹¹S(O)₂R¹⁴—NR¹¹C(O)OR³, —NR¹¹C(O)R¹², —C(NR¹¹)NR¹⁰R¹¹, —C(O)NR¹⁰R¹¹, —C(O)OR¹³, —CN, —NO₂, and —C(O)R¹²;

R³ is absent or is selected from hydrogen, halogen, optionally substituted alkyl, optionally substituted alkenyl, optionally substituted alkynyl, optionally substituted cycloalkyl, optionally substituted amino, optionally substituted heterocycloalkyl, optionally substituted aryl, optionally substituted heteroaryl, —OR¹¹, —SR¹⁵, —S(O)R¹⁶, —S(O)₂R¹⁶, —S(O)₂NR¹⁰R¹¹, —NR¹⁰R¹¹, —NR¹¹C(O)NR¹⁰R¹¹, —NR¹¹C(S)NR¹⁰R¹¹, —NR¹¹S(O)₂R¹⁴—NR¹¹C(O)OR³, —NR¹¹C(O)R¹² —C(NR¹¹)NR¹⁰R¹¹, —C(O)NR¹⁰R¹¹, —C(O)OR¹³, —CN, —NO₂, and —C(O)R¹²;

R⁴ is selected from hydrogen, halogen, optionally substituted alkyl, optionally substituted alkenyl, optionally substituted alkynyl, optionally substituted cycloalkyl, optionally substituted amino, optionally substituted heterocycloalkyl, optionally substituted aryl, optionally substituted heteroaryl, —OR¹⁵, —SR¹⁵, —S(O)R¹⁶, —S(O)₂R¹⁶, —S(O)₂NR¹⁰R¹¹, —NR¹⁰R¹¹, —NR¹¹C(O)NR¹⁰R¹¹, —NR¹¹C(S)NR¹⁰R¹¹, —NR¹¹S(O)₂R¹⁴, —NR¹¹C(O)OR¹³, —NR¹¹C(O)R¹², —C(NR¹¹)NR¹⁰R¹¹, —C(O)NR¹⁰R¹¹, —C(O)OR¹³, —CN, —NO₂, and —C(O)R¹²;

R⁵ is selected from halogen, optionally substituted alkyl, optionally substituted alkenyl, optionally substituted alkynyl, optionally substituted cycloalkyl, optionally substituted amino, optionally substituted heterocycloalkyl, optionally substituted aryl, optionally substituted heteroaryl, —OR¹⁵, —SR¹⁵, —S(O)R¹⁶, —S(O)₂R¹⁶, —S(O)₂NR¹⁰R¹¹, —NR¹⁰R¹¹, —NR¹¹C(O)NR¹⁰R¹¹, —NR¹¹C(S)NR¹⁰R¹¹, —NR¹¹S(O)₂R¹⁴—NR¹¹C(O)OR³, —NR¹¹C(O)R¹², —C(NR¹¹)NR¹⁰R¹¹, —C(O)NR¹⁰R¹¹, —C(O)OR¹³, —CN, —NO₂, and —C(O)R¹²;

R⁶ is selected from hydrogen, halogen, optionally substituted alkyl, optionally substituted alkenyl, optionally substituted alkynyl, optionally substituted cycloalkyl, optionally substituted amino, optionally substituted heterocycloalkyl, optionally substituted aryl, optionally substituted heteroaryl, —OR¹⁵, —SR¹⁵, —S(O)R¹⁶, —S(O)₂R¹⁶, —S(O)₂NR¹⁰R¹¹, —NR¹⁰R¹¹, —NR¹¹C(O)NR¹⁰R¹¹, —NR¹¹C(S)NR¹⁰R¹¹, —NR¹¹S(O)₂R¹⁴—NR¹¹C(O)OR³, —NR¹¹C(O)R¹², —C(NR¹¹)NR¹⁰R¹¹, —C(O)NR¹⁰R¹¹, —C(O)OR¹³, —CN, —NO₂, and —C(O)R¹²;

R⁷ is selected from halogen, optionally substituted alkyl, optionally substituted alkenyl, optionally substituted alkynyl, optionally substituted cycloalkyl, optionally substituted amino, optionally substituted heterocycloalkyl, optionally substituted aryl, optionally substituted heteroaryl, —OR¹⁵, —SR¹⁵, —S(O)R¹⁶, —S(O)₂R¹⁶, —S(O)₂NR¹⁰R¹¹, —NR¹⁰R¹¹, —NR¹¹C(O)NROR¹¹, —NR C(S)NR¹⁰R¹¹, —NR¹¹S(O)₂R¹⁴—NR¹¹C(O)OR¹³, —NR¹¹C(O)R¹², —C(NR¹¹)NR¹⁰R¹¹, —C(O)NR¹⁰R¹¹, —C(O)OR¹³, —CN, —NO₂, and —C(O)R¹²;

R¹⁰ and R¹¹ are independently selected from hydrogen, optionally substituted alkyl, optionally substituted amino, optionally substituted alkoxy, optionally substituted cycloalkyl, optionally substituted heterocycloalkyl, optionally substituted aryl, and optionally substituted heteroaryl, or R¹⁰ and R¹¹, taken together with any intervening atoms, form a ring system selected from optionally substituted heterocycloalkyl, and optionally substituted heteroaryl;

R¹² is selected from hydrogen, optionally substituted alkyl, optionally substituted cycloalkyl, optionally substituted heterocycloalkyl, optionally substituted aryl, and optionally substituted heteroaryl;

R¹³ is selected from hydrogen, optionally substituted alkyl, optionally substituted cycloalkyl, optionally substituted heterocycloalkyl, optionally substituted aryl, and optionally substituted heteroaryl;

R¹⁴ is selected from optionally substituted alkyl, optionally substituted cycloalkyl, optionally substituted heterocycloalkyl, optionally substituted aryl, and optionally substituted heteroaryl;

R¹⁵ is selected from hydrogen, optionally substituted alkyl, optionally substituted cycloalkyl, optionally substituted heterocycloalkyl, optionally substituted aryl, and optionally substituted heteroaryl; and

R¹⁶ is selected from optionally substituted alkyl, optionally substituted cycloalkyl, optionally substituted heterocycloalkyl, optionally substituted aryl, and optionally substituted heteroaryl;

provided that
if W¹ is NR¹ and W³ is NR³, then R³ is absent;
if W³ is NR³ and W¹ is NR¹, then R¹ is absent;
at least one of W¹, W³, W⁸, and W⁹ is N;
no more than four of W¹, W³, W⁴, W⁶, W⁸, and W⁹ are N; and
if W¹ is N, W⁴ is N, and W⁶ is CR⁶, then W⁸ is not N; and further provided that the compound of Formula 1 is not
(5-(5-chlorothiophen-2-yl)-7-(trifluoromethyl)pyrazolo[1,5-a]pyridin-2-yl)(3-(3,4-dimethoxyphenyl)-5-(2-hydroxyphenyl)-4,5-dihydro-1H-pyrazol-1-yl)methanone;
(5-(5-chlorothiophen-2-yl)-7-(trifluoromethyl)pyrazolo[1,5-a]pyridin-2-yl)(3-(2,5-dimethylphenyl)-5-(2-hydroxyphenyl)-4,5-dihydro-1H-pyrazol-1-yl)methanone; or
(5-(5-chlorothiophen-2-yl)-7-(trifluoromethyl)pyrazolo[1,5-a]pyridin-2-yl)(3-(3,4-dichlorophenyl)-5-(2-hydroxyphenyl)-4,5-dihydro-1H-pyrazol-1-yl)methanone.

In some embodiments, the compound of Formula 1 is selected from the following compounds:

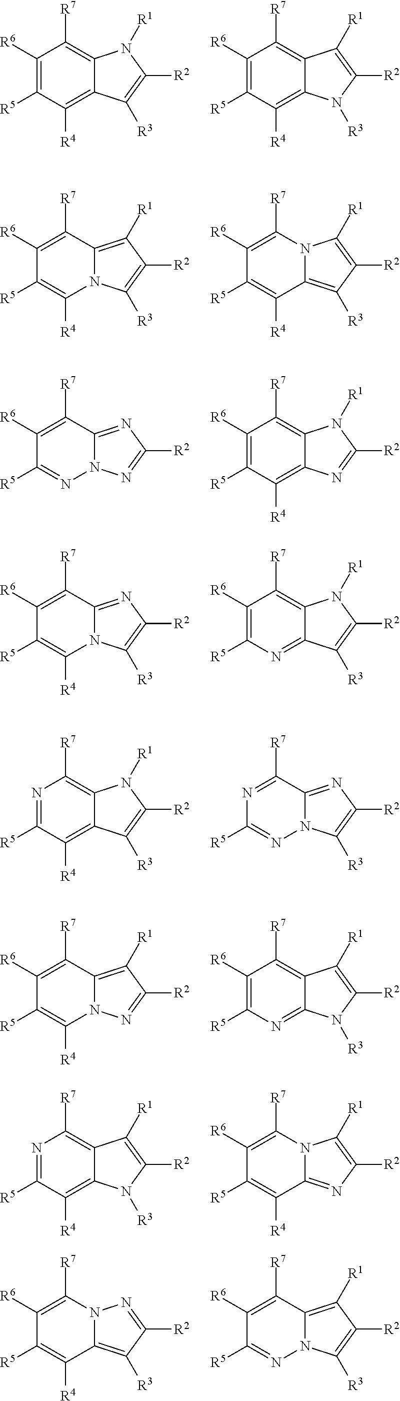

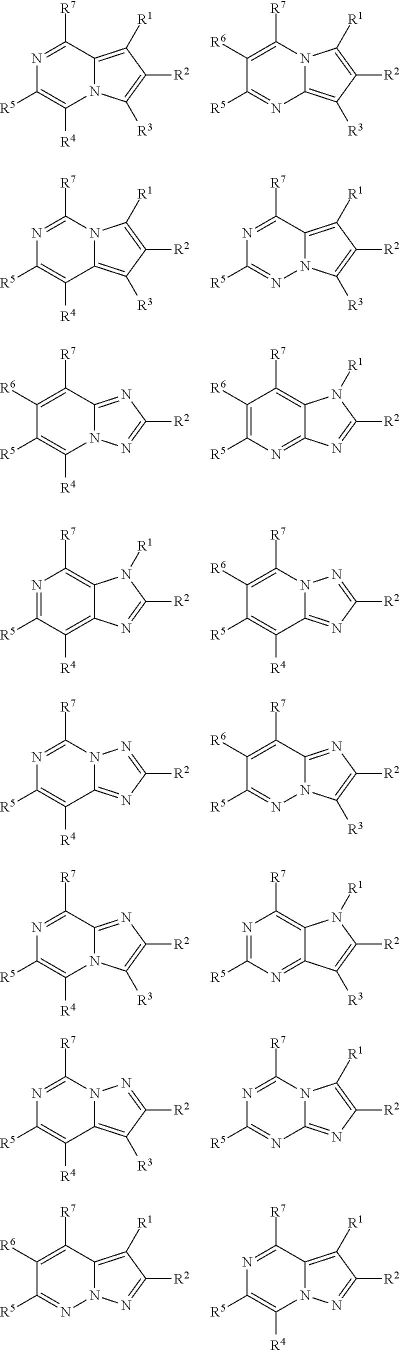

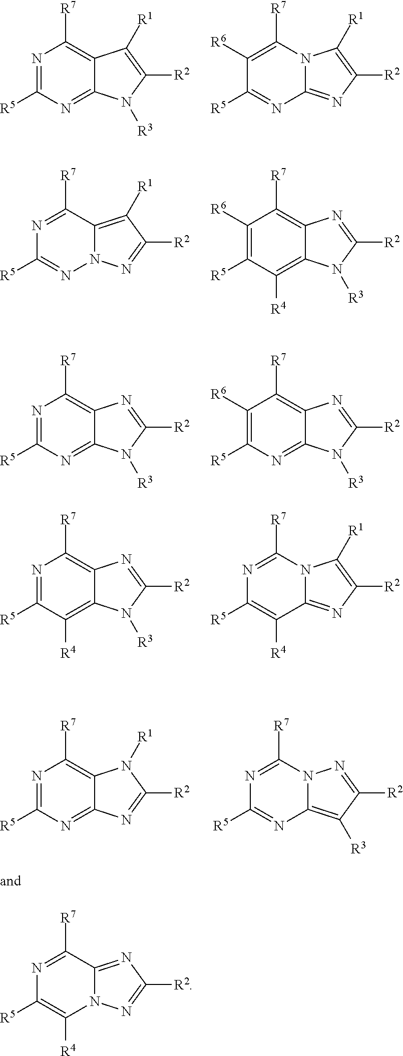

In some embodiments, the compound of Formula 1 is selected from the following compounds:

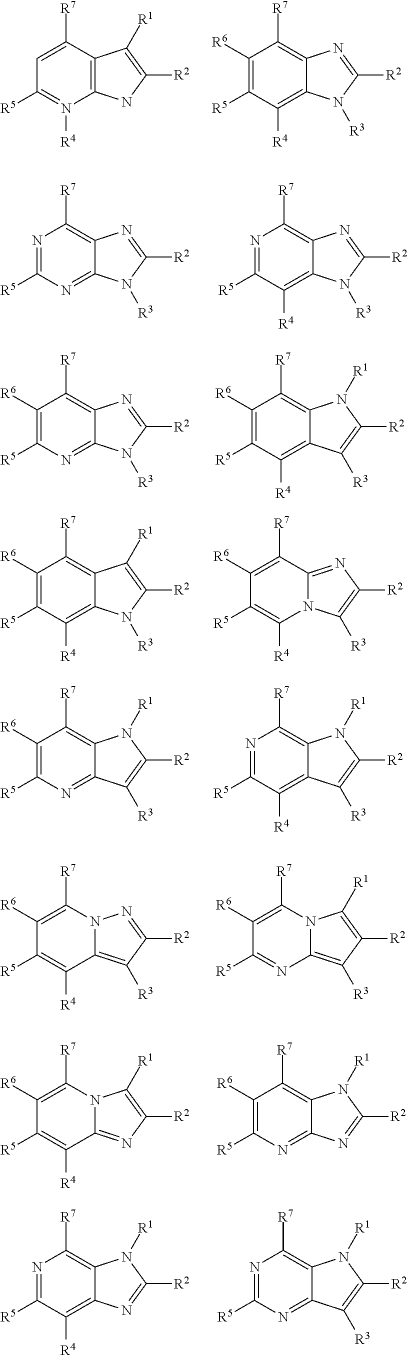

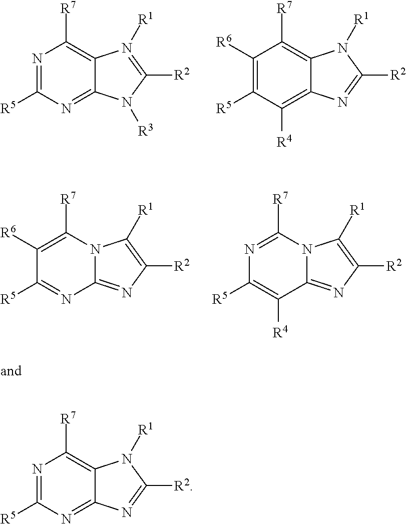

In some embodiments, the compound of Formula 1 is selected from the following compounds:

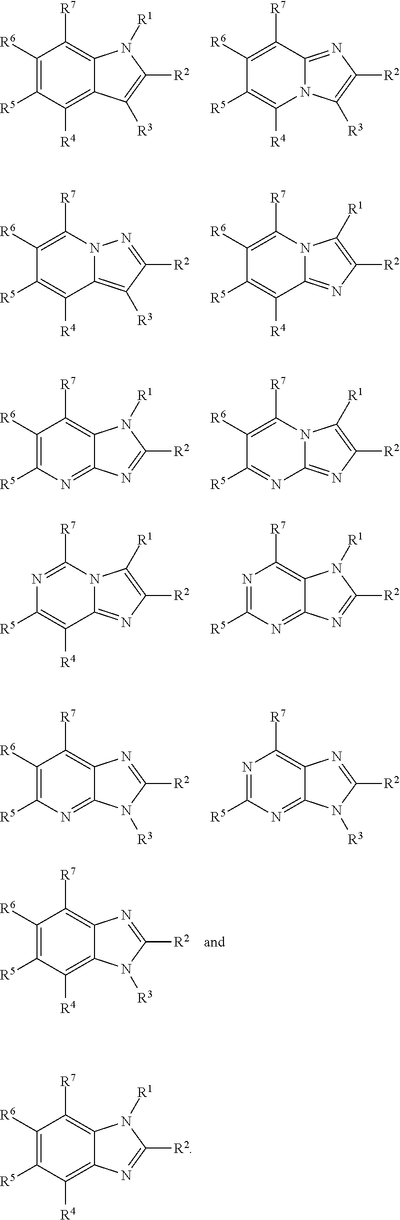

In some embodiments, the compound of Formula 1 is selected from the following compounds:

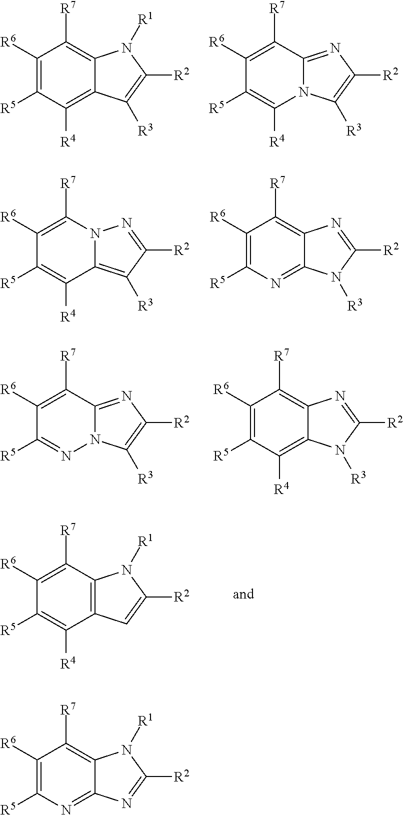

In some embodiments, the compound of Formula 1 is selected from the following compounds:

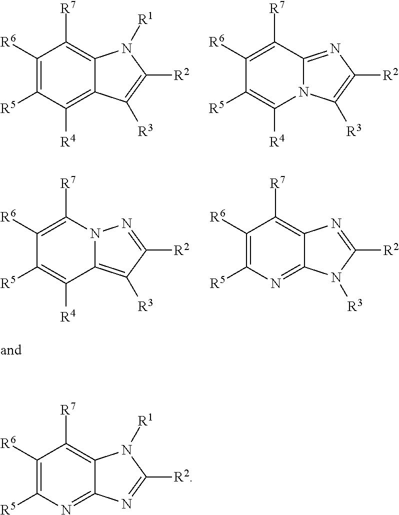

In some embodiments, the compound of Formula 1 is

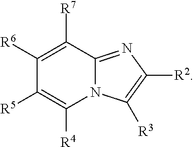

In some embodiments, R² is selected from optionally substituted alkyl, —NR¹¹S(O)₂R¹⁴, —NR¹¹C(O)NR¹⁰R¹¹, —NR¹¹C(O)OR³—C(O)NR¹⁰R¹¹, and —C(O)OR¹³.

In some embodiments, R² is lower alkyl substituted with —NR¹⁰R¹¹, where R¹⁰ and R¹¹ are as described herein. In some embodiments, R² is —CH₂—NR¹⁰R¹¹, where R¹⁰ and R¹¹ are as described herein.

In some embodiments, R² is lower alkyl substituted with —NR¹⁰R¹¹ and R¹⁰ and R¹¹, together with any intervening atoms, form an optionally substituted heterocycloalkyl, as described herein. In some embodiments, R² is —CH₂—NR¹⁰R¹¹ and R¹⁰ and R¹¹, together with any intervening atoms, form an optionally substituted heterocycloalkyl, as described herein.

In some embodiments, R² is lower alkyl substituted with —C(O)NR¹⁰R¹¹, where R¹⁰ and R¹¹ are as described herein. In some embodiments, R² is —CH₂—C(O)NR¹⁰R¹¹, where R¹⁰ and R¹¹ are as described herein.

In some embodiments, R² is —C(O)NR¹⁰R¹¹.

In some embodiments, R¹⁰ is selected from lower alkyl and hydrogen. In some embodiments, R¹⁰ is selected from optionally substituted alkyl, optionally substituted cycloalkyl, optionally substituted heterocycloalkyl, and optionally substituted aryl. In some embodiments, R¹⁰ is —(CR¹⁷R¹⁸)ₙR¹⁹, wherein R¹⁷ and R¹⁸ are independently selected from hydrogen, carboxy, optionally substituted aminocarbonyl, lower carboxy ester, and lower alkyl; n is 0, 1 or 2; and R¹⁹ is chosen from optionally substituted aryl and optionally substituted heteroaryl. In some embodiments, R¹⁰ is benzyl, thiophen-2-yl-ethyl, thiophen-3-yl-methyl, furan-2-yl-methyl, and furan-3-yl-methyl, each of which is optionally substituted. In some embodiments, R¹¹ is selected from lower alkyl and hydrogen.

In some embodiments, R¹⁰ and R¹¹, together with any intervening atoms, form an optionally substituted heterocycloalkyl. In some embodiments, R¹⁰ and R¹¹, together with any intervening atoms, form a substituted 3- to 7-membered nitrogen containing heterocycloalkyl which optionally further includes one or two additional heteroatoms chosen from N, O, S, S(O), S(O)₂, and P(O), wherein said 3- to 7-membered nitrogen containing heterocycloalkyl is substituted with a group —Y—R³⁰ and optionally substituted with a second group R³¹, wherein

Y is a bond or is selected from —NR¹⁰—, —NR¹¹SO₂—, —O—, —S—, —C(O)NR¹⁰—, and —S(O)₂R¹⁰—;

R³⁰ is selected from optionally substituted alkyl, optionally substituted cycloalkyl, optionally substituted heterocycloalkyl, optionally substituted aryl, and optionally substituted heteroaryl; and

R³¹ is selected from halogen, optionally substituted alkyl, optionally substituted alkenyl, optionally substituted alkynyl, optionally substituted cycloalkyl, optionally substituted heterocycloalkyl, optionally substituted aryl, optionally substituted heteroaryl, optionally substituted alkoxy, —OH, —SH, —NO₂, —NR¹⁰R¹¹, —C(O)NR¹⁰R¹¹, —C(O)OR¹³, —SO₂NR¹⁰R¹¹, —NR¹¹C(S)NR¹⁰R¹¹, —NR¹¹C(O)NR¹⁰R¹¹, —CN, —NR¹¹SO₂R¹⁴, and —NR¹¹CO₂R¹³.

In some embodiments, R¹⁰ and R¹¹, together with any intervening atoms, form a substituted 3- to 7-membered nitrogen containing heterocycloalkyl which optionally further includes one or two additional heteroatoms chosen from N, O, S, S(O), S(O)₂, and P(O), wherein said 3- to 7-membered nitrogen containing heterocycloalkyl is substituted with a group —Y—R³⁰ and optionally substituted with a second group R³¹, wherein

Y is a bond or is selected from —O—, —S—, —C(O)NR¹⁰, and —S(O)₂R¹⁰—;

R³⁰ is selected from optionally substituted cycloalkyl, optionally substituted heterocycloalkyl, optionally substituted aryl, and optionally substituted heteroaryl; and

R³¹ is selected from halogen, optionally substituted alkyl, optionally substituted alkenyl, optionally substituted alkynyl, optionally substituted cycloalkyl, optionally substituted heterocycloalkyl, optionally substituted aryl, optionally substituted heteroaryl, optionally substituted alkoxy, —NO₂, —NR¹⁰R¹¹, —C(O)NR¹⁰R¹¹, —C(O)OR¹³, —SO₂NR¹⁰R¹¹, —NR¹¹C(S)NR¹⁰R¹¹, —NR¹¹C(O)NR¹⁰R¹¹, —CN, —NR¹¹SO₂R¹⁴, and —NR¹¹CO₂R¹³.

In some embodiments, Y is a bond or is selected from —NR¹⁰— and —O—. In some embodiments, Y is a bond or is —O—. In some embodiments, Y is a bond.

In some embodiments, R³⁰ is selected from optionally substituted aryl and optionally substituted heteroaryl. In some embodiments, R³⁰ is selected from phenyl, thiophen-2-yl, thiophen-3-yl, furan-2-yl, furan-3-yl, thiazol-2-yl, thiazol-4-yl, thiazol-5-yl, pyrazol-4-yl, imidazol-4-yl, and imidazol-2-yl. In some embodiments, R³⁰ is selected from phenyl, thiophen-2-yl, thiophen-3-yl, furan-2-yl, and furan-3-yl. In some embodiments, R³⁰ is phenyl. In some embodiments, R³⁰ is optionally substituted alkyl. In some embodiments, R³⁰ is optionally substituted lower alkyl. In some embodiments, R³⁰ is lower alkyl. In some embodiments, R³⁰ is methyl.

In some embodiments, R² is —C(O)NR¹⁰R¹¹ and R¹⁰ and R¹¹, together with any intervening atoms, form a pyrrolidinyl, piperidinyl, piperazinyl, 5,6-dihydropyridin-1(2H)-yl, 4,5-dihydro-1H-pyrazol-1-yl, 2,5-dihydro-1H-pyrrol-1-yl, or azetidinyl ring, wherein said ring is substituted with a group —Y—R³⁰ and optionally substituted with a second group R³¹ as described above.

In some embodiments, R² is lower alkyl substituted with —C(O)NR¹⁰R¹¹ and R¹⁰ and R¹¹, together with any intervening atoms, form a pyrrolidinyl, piperidinyl, piperazinyl, 5,6-dihydropyridin-1(2H)-yl, 4,5-dihydro-1H-pyrazol-1-yl, 2,5-dihydro-H-pyrrol-1-yl, or azetidinyl ring, wherein said ring is substituted with a group —Y—R³⁰ and optionally substituted with a second group R³¹ as described above. In some embodiments, R² is —CH₂— substituted with —C(O)NR¹⁰R¹¹ and R¹⁰ and R¹¹, together with any intervening atoms, form a pyrrolidinyl, piperidinyl, piperazinyl, 5,6-dihydropyridin-1(2H)-yl, 4,5-dihydro-1H-pyrazol-1-yl, 2,5-dihydro-1H-pyrrol-1-yl, or azetidinyl ring, wherein said ring is substituted with a group —Y—R³⁰ and optionally substituted with a second group R³¹ as described above.

In some embodiments, R² is optionally substituted heteroaryl. In some embodiments, R² is isoxazol-5-yl or [1,2,4]oxadiazol-5-yl, each of which is optionally substituted. In some embodiments, R² is isoxazol-5-yl or [1,2,4]oxadiazol-5-yl, each of which is optionally substituted with a group chosen from optionally substituted aryl and optionally substituted alkyl. In some embodiments, R² is isoxazol-5-yl or [1,2,4]oxadiazol-5-yl, each of which is optionally substituted with a group chosen from optionally substituted phenyl, optionally substituted benzyl, and optionally substituted phenoxymethyl. In some embodiments, R² is isoxazol-5-yl or [1,2,4]oxadiazol-5-yl, each of which is optionally substituted with a group chosen from phenyl, benzyl, and phenoxymethyl.

In some embodiments, R³ is selected from optionally substituted alkyl and halogen. In some embodiments, R³ is selected from lower alkyl and halogen. In some embodiments, R³ is halogen. In some embodiments, R³ is selected from chlorine and bromine. In some embodiments, R³ is chlorine. In some embodiments, R³ is hydrogen.

In some embodiments, R⁴ is selected from hydrogen, optionally substituted alkyl, —NR¹¹SO₂R¹⁴, —NR¹¹C(O)NR¹⁰R¹¹, —NR¹¹CO₂R¹³—S(O)NR¹⁰R¹¹, —NR¹⁰C(O)NR¹⁰R¹¹, —CN, —NO₂, and —C(O)R¹². In some embodiments, R¹¹ is hydrogen. In some embodiments, R¹⁰ is selected from optionally substituted alkyl and optionally substituted cycloalkyl.

In some embodiments, R⁴ is selected from hydrogen and optionally substituted lower alkyl. In some embodiments, R⁴ is hydrogen.

In some embodiments, R⁴ is —CN.

In some embodiments, R⁵ is selected from optionally substituted cycloalkyl, optionally substituted aryl, optionally substituted heteroaryl, and optionally substituted heterocycloalkyl. In some embodiments, R⁵ is selected from optionally substituted cycloalkyl, optionally substituted aryl, and optionally substituted heteroaryl. In some embodiments, R⁵ is selected from optionally substituted aryl and optionally substituted heteroaryl. In some embodiments, R⁵ is selected from pyrid-3-yl, pyrazol-4-yl, phenyl, furan-2-yl, furan-3-yl, thiophen-2-yl, and thiophen-3-yl, each of which is optionally substituted. In some embodiments, R⁵ is selected from phenyl, furan-2-yl, furan-3-yl, thiophen-2-yl, and thiophen-3-yl, each of which is optionally substituted. In some embodiments, R⁵ is selected from phenyl, furan-2-yl, furan-3-yl, thiophen-2-yl, and thiophen-3-yl, each of which is optionally substituted with one or two groups chosen from lower alkyl, halogen, morpholinyl, trifluoromethyl, and lower alkoxy. In some embodiments, R⁵ is selected from phenyl, 3-fluorophenyl, furan-2-yl, furan-3-yl, thiophen-2-yl, and thiophen-3-yl.

In some embodiments, R⁶ is selected from hydrogen, halogen, optionally substituted alkyl, —OR¹⁵, —S(O)NR¹⁰R¹¹, —C(O)R¹², —NO₂, —C(O)NR¹⁰R¹¹, and —NR¹⁰R¹¹. In some embodiments, R⁶ is selected from hydrogen, halogen, optionally substituted alkyl, —S(O)NR¹⁰R¹¹, —C(O)R¹², —NO₂, —C(O)NR¹⁰R¹¹, and —NR¹⁰R¹¹. In some embodiments, R¹¹ is hydrogen. In some embodiments, R¹⁰ is selected from optionally substituted alkyl and optionally substituted cycloalkyl. In some embodiments, R¹⁰ and R¹¹, taken together with any intervening atoms, form an optionally substituted heterocycloalkyl ring.

In some embodiments, R⁶ is selected from hydrogen, halogen, and optionally substituted alkyl. In some embodiments, R⁶ is selected from hydrogen and halogen. In some embodiments, R⁶ is hydrogen.

In some embodiments, R⁷ is selected from halogen, optionally substituted alkyl, optionally substituted cycloalkyl, optionally substituted alkoxy, heterocycloalkyl, optionally substituted aryl, —SO₂NR¹⁰R¹¹, and —NR¹⁰R¹¹. In some embodiments, R⁷ is selected from halogen, optionally substituted alkyl, optionally substituted cycloalkyl, optionally substituted alkoxy, heterocycloalkyl, optionally substituted aryl, and —NR¹⁰R¹¹. In some embodiments, R⁷ is selected from optionally substituted alkyl, optionally substituted cycloalkyl, optionally substituted alkoxy, and —NR¹⁰R¹¹. In some embodiments, R⁷ is selected from optionally substituted alkyl, optionally substituted alkoxy, and —NR¹⁰R¹¹. In some embodiments, R⁷ is selected from optionally substituted lower alkoxy and optionally substituted lower alkyl.

In some embodiments, R⁷ is polyhalogenated lower alkoxy. In some embodiments, R⁷ selected from trifluoromethoxy and difluorochloromethoxy.

In some embodiments, R⁷ is polyhalogenated lower alkyl. In some embodiments, R⁷ is polyhalogenated methyl. In some embodiments, R⁷ is selected from trifluoromethyl and difluorochloromethyl. In some embodiments, R⁷ is trifluoromethyl.

In some embodiments, R⁷ is —NR¹⁰R¹¹. In some embodiments, R¹¹ is hydrogen. In some embodiments, R¹⁰ is optionally substituted lower alkyl. In some embodiments, R¹⁰ is methyl. In some embodiments, R¹⁰ is 2-hydroxyethyl.

In some embodiments, the compound of Formula 1 is chosen from the compounds set forth in Table 1, Table 2, and Table 3.

TABLE 1

| Compound | | |
|---|---|---|
| Number | Structure | Compound Name |
| 102 | 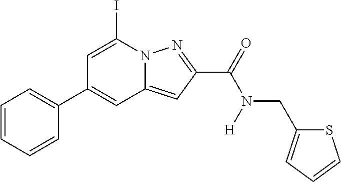 | 7-Iodo-5-phenyl- pyrazolo[1,5-a]pyridine-2- carboxylic acid (thiophen- 2-ylmethyl)-amide |
| 103 | 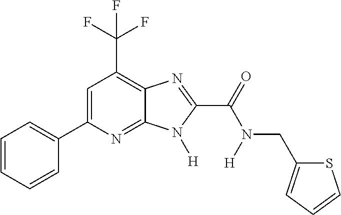 | 5-Phenyl-7- trifluoromethyl-3H- imidazo[4,5-b]pyridine-2- carboxylic acid (thiophen- 2-ylmethyl)-amide |
| 104 | 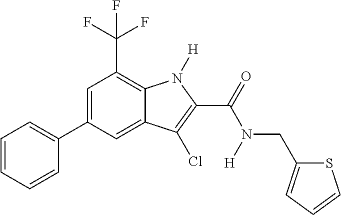 | 3-Chloro-5-phenyl-7- trifluoromethyl-1H-indole- 2-carboxylic acid (thiophen-2-ylmethyl)- amide |
| 105 | 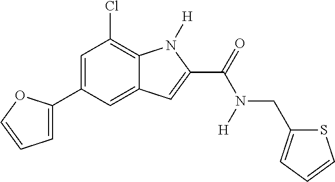 | 7-Chloro-5-furan-2-yl-1H- indole-2-carboxylic acid (thiophen-2-ylmethyl)- amide |
| 106 | 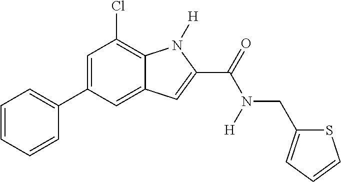 | 7-Chloro-5-phenyl-1H- indole-2-carboxylic acid (thiophen-2-ylmethyl)- amide |
| 107 | 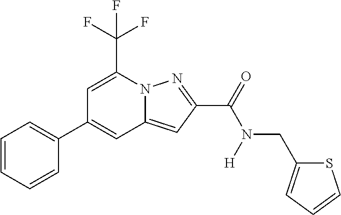 | 5-Phenyl-7- trifluoromethyl- pyrazolo[1,5-a]pyridine-2- carboxylic acid (thiophen- 2-ylmethyl)-amide |
| 108 | 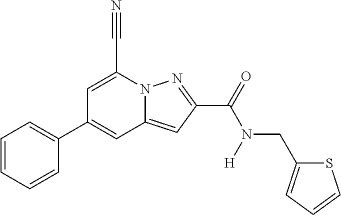 | 7-Cyano-5-phenyl- pyrazolo[1,5-a]pyridine-2- carboxylic acid (thiophen- 2-ylmethyl)-amide |
| 109 | 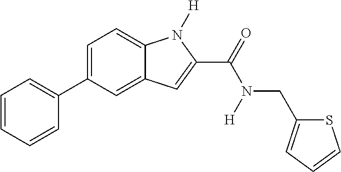 | 5-Phenyl-1H-indole-2- carboxylic acid (thiophen- 2-ylmethyl)-amide |
| 110 | 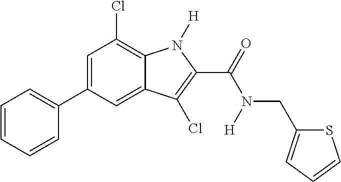 | 3,7-Dichloro-5-phenyl-1H- indole-2-carboxylic acid (thiophen-2-ylmethyl)- amide |
| 111 | 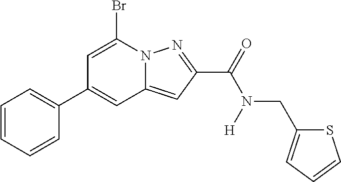 | 7-Bromo-5-phenyl- pyrazolo[1,5-a]pyridine-2- carboxylic acid (thiophen- 2-ylmethyl)-amide |
| 112 | 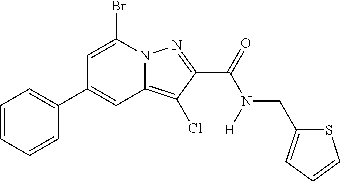 | 7-Bromo-3-chloro-5- phenyl-pyrazolo[1,5- a]pyridine-2-carboxylic acid (thiophen-2- ylmethyl)-amide |
| 113 | 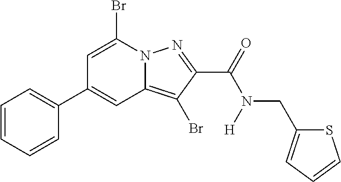 | 3,7-Dibromo-5-phenyl- pyrazolo[1,5-a]pyridine-2- carboxylic acid (thiophen- 2-ylmethyl)-amide |
| 114 | 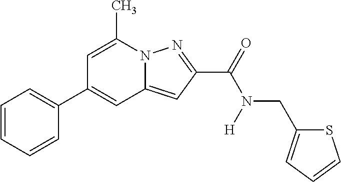 | 7-Methyl-5-phenyl- pyrazolo[1,5-a]pyridine-2- carboxylic acid (thiophen- 2-ylmethyl)-amide |
| 115 | 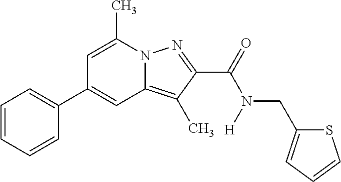 | 3,7-Dimethyl-5-phenyl- pyrazolo[1,5-a]pyridine-2- carboxylic acid (thiophen- 2-ylmethyl)-amide |
| 116 | 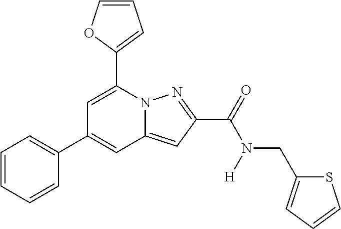 | 7-Furan-2-yl-5-phenyl- pyrazolo[1,5-a]pyridine-2- carboxylic acid (thiophen- 2-ylmethyl)-amide |
| 117 | 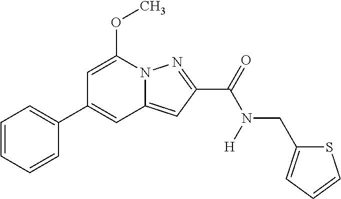 | 7-Methoxy-5-phenyl- pyrazolo[1,5-a]pyridine-2- carboxylic acid (thiophen- 2-ylmethyl)-amide |
| 118 | 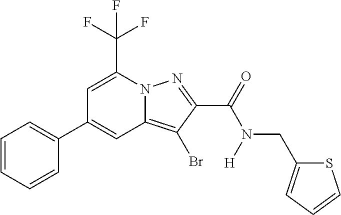 | 3-Bromo-5-phenyl-7- trifluoromethyl- pyrazolo[1,5-a]pyridine-2- carboxylic acid (thiophen- 2-ylmethyl)-amide |
| 119 | 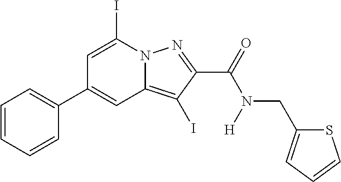 | 3,7-Diiodo-5-phenyl- pyrazolo[1,5-a]pyridine-2- carboxylic acid (thiophen- 2-ylmethyl)-amide |
| 120 | 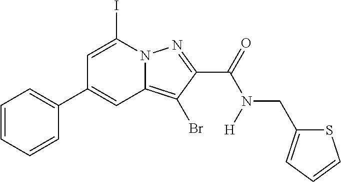 | 3-Bromo-7-iodo-5-phenyl- pyrazolo[1,5-a]pyridine-2- carboxylic acid (thiophen- 2-ylmethyl)-amide |
| 121 | 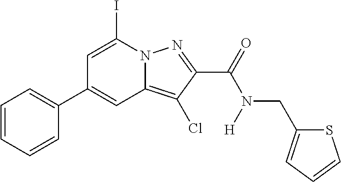 | 3-Chloro-7-iodo-5-phenyl- pyrazolo[1,5-a]pyridine-2- carboxylic acid (thiophen- 2-ylmethyl)-amide |
| 122 | 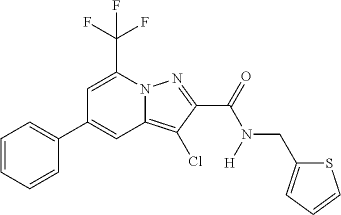 | 3-Chloro-5-phenyl-7- trifluoromethyl- pyrazolo[1,5-a]pyridine-2- carboxylic acid (thiophen- 2-ylmethyl)-amide |
| 123 | 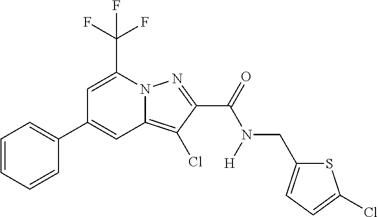 | 3-Chloro-5-phenyl-7- trifluoromethyl- pyrazolo[1,5-a]pyridine-2- carboxylic acid (5-chloro- thiophen-2-ylmethyl)- amide |
| 124 | 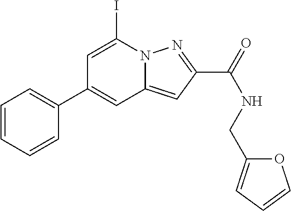 | 7-Iodo-5-phenyl- pyrazolo[1,5-a]pyridine-2- carboxylic acid (furan-2- ylmethyl)-amide |
| 125 | 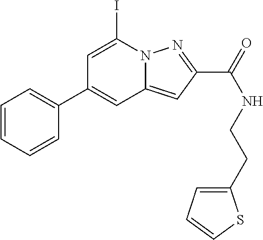 | 7-Iodo-5-phenyl- pyrazolo[1,5-a]pyridine-2- carboxylic acid (2- thiophen-2-yl-ethyl)- amide |
| 126 | 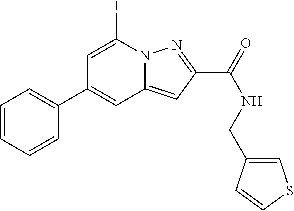 | 7-Iodo-5-phenyl- pyrazolo[1,5-a]pyridine-2- carboxylic acid (thiophen- 3-ylmethyl)-amide |
| 127 | 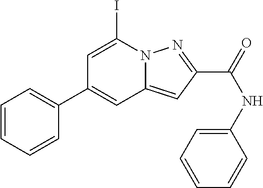 | 7-Iodo-5-phenyl- pyrazolo[1,5-a]pyridine-2- carboxylic acid phenylamide |
| 128 | 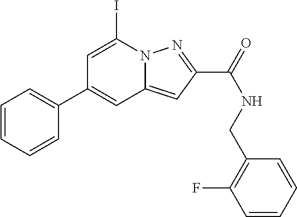 | 7-Iodo-5-phenyl- pyrazolo[1,5-a]pyridine-2- carboxylic acid 2-fluoro- benzylamide |
| 129 | 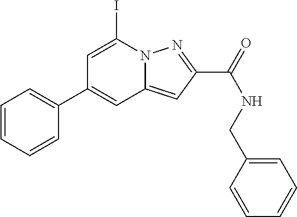 | 7-Iodo-5-phenyl- pyrazolo[1,5-a]pyridine-2- carboxylic acid benzylamide |
| 130 | 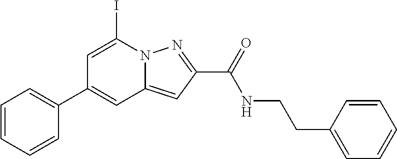 | 7-Iodo-5-phenyl- pyrazolo[1,5-a]pyridine-2- carboxylic acid phenethyl- amide |
| 131 | 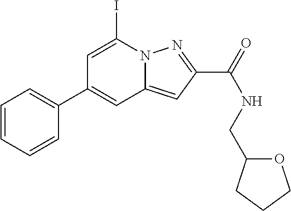 | 7-Iodo-5-phenyl- pyrazolo[1,5-a]pyridine-2- carboxylic acid (tetrahydro-furan-2- ylmethyl)-amide |
| 132 | 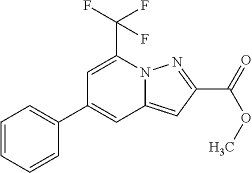 | 5-Phenyl-7- trifluoromethyl- pyrazolo[1,5-a]pyridine-2- carboxylic acid methyl ester |
| 133 | 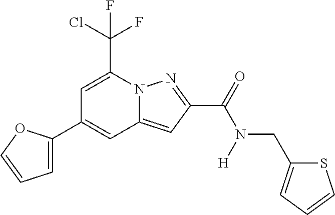 | 7-(Chloro-difluoro- methyl)-5-furan-2-yl- pyrazolo[1,5-a]pyridine-2- carboxylic acid (thiophen- 2-ylmethyl)-amide |
| 134 | 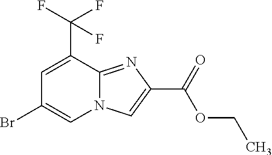 | 6-Bromo-8- trifluoromethyl- imidazo[1,2-a]pyridine-2- carboxylic acid ethyl ester |
| 135 | 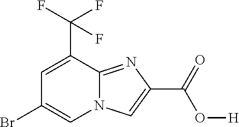 | 6-Bromo-8- trifluoromethyl- imidazo[1,2-a]pyridine-2- carboxylic acid |
| 136 | 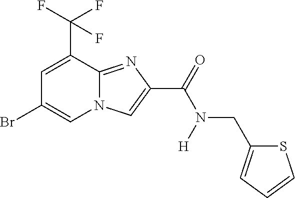 | 6-Bromo-8- trifluoromethyl- imidazo[1,2-a]pyridine-2- carboxylic acid (thiophen- 2-ylmethyl)-amide |
| 137 | 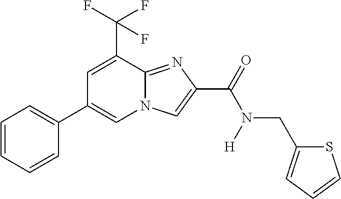 | 6-Phenyl-8- trifluoromethyl- imidazo[1,2-a]pyridine-2- carboxylic acid (thiophen- 2-ylmethyl)-amide |
| 138 | 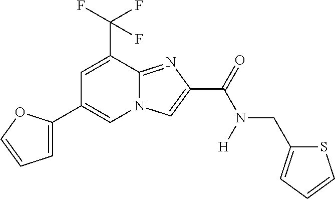 | 6-Furan-2-yl-8- trifluoromethyl- imidazo[1,2-a]pyridine-2- carboxylic acid (thiophen- 2-ylmethyl)-amide |
| 139 | 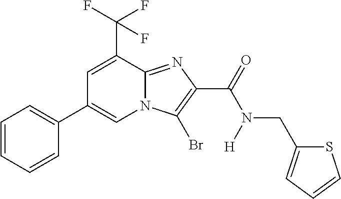 | 3-Bromo-6-phenyl-8- trifluoromethyl- imidazo[1,2-a]pyridine-2- carboxylic acid (thiophen- 2-ylmethyl)-amide |
| 140 | 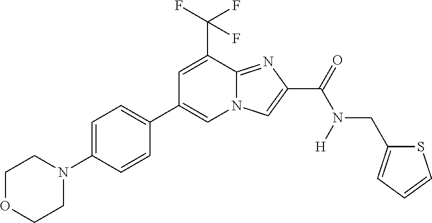 | 6-(4-Morpholin-4-yl- phenyl)-8-trifluoromethyl- imidazo[1,2-a]pyridine-2- carboxylic acid (thiophen- 2-ylmethyl)-amide |
| 141 | 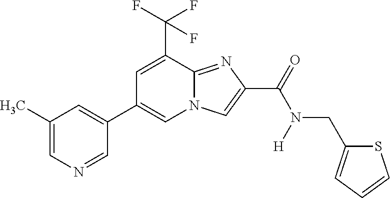 | 6-(5-Methyl-pyridin-3-yl)- 8-trifluoromethyl- imidazo[1,2-a]pyridine-2- carboxylic acid (thiophen- 2-ylmethyl)-amide |
| 142 | 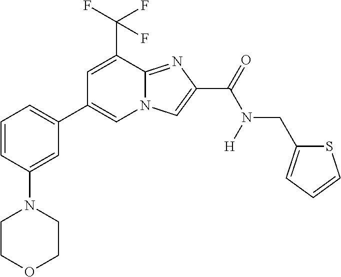 | 6-(3-Morpholin-4-yl- phenyl)-8-trifluoromethyl- imidazo[1,2-a]pyridine-2- carboxylic acid (thiophen- 2-ylmethyl)-amide |
| 143 | 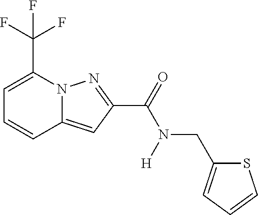 | 7-Trifluoromethyl- pyrazolo[1,5-a]pyridine-2- carboxylic acid (thiophen- 2-ylmethyl)-amide |
| 144 | 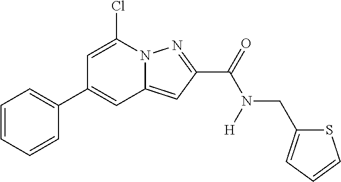 | 7-Chloro-5-phenyl- pyrazolo[1,5-a]pyridine-2- carboxylic acid (thiophen- 2-ylmethyl)-amide |
| 145 | 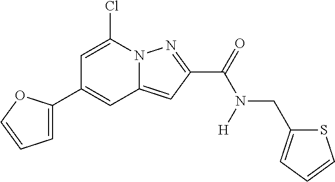 | 7-Chloro-5-furan-2-yl- pyrazolo[1,5-a]pyridine-2- carboxylic acid (thiophen- 2-ylmethyl)-amide |
| 146 | 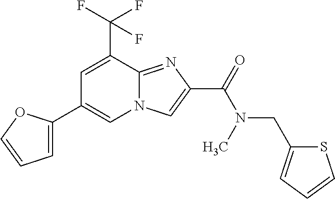 | 6-Furan-2-yl-8- trifluoromethyl- imidazo[1,2-a]pyridine-2- carboxylic acid methyl- thiophen-2-ylmethyl- amide |
| 147 | 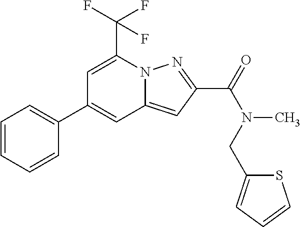 | 5-Phenyl-7- trifluoromethyl- pyrazolo[1,5-a]pyridine-2- carboxylic acid methyl- thiophen-2-ylmethyl- amide |
| 148 | 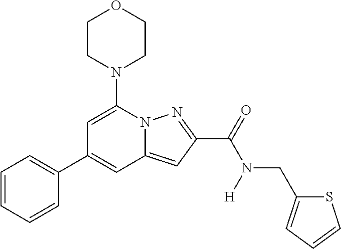 | 7-Morpholin-4-yl-5- phenyl-pyrazolo[1,5- a]pyridine-2-carboxylic acid (thiophen-2- ylmethyl)-amide |
| 149 | 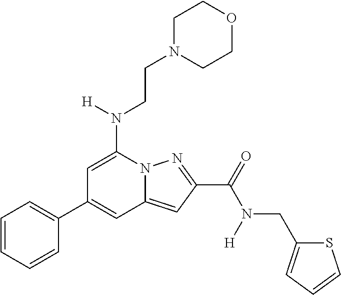 | 7-(2-Morpholin-4-yl- ethylamino)-5-phenyl- pyrazolo[1,5-a]pyridine-2- carboxylic acid (thiophen- 2-ylmethyl)-amide |
| 150 | 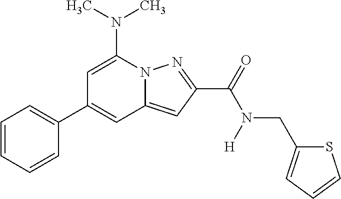 | 7-Dimethylamino-5- phenyl-pyrazolo[1,5- a]pyridine-2-carboxylic acid (thiophen-2- ylmethyl)-amide |
| 151 | 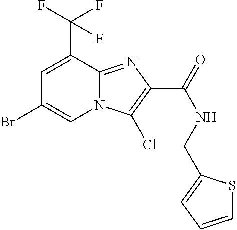 | 6-Bromo-3-chloro-8- trifluoromethyl- imidazo[1,2-a]pyridine-2- carboxylic acid (thiophen- 2-ylmethyl)-amide |
| 152 | 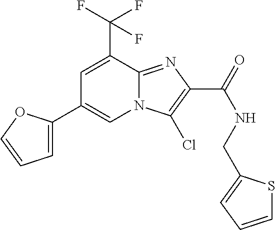 | 3-Chloro-6-furan-2-yl-8- trifluoromethyl- imidazo[1,2-a]pyridine-2- carboxylic acid (thiophen- 2-ylmethyl)-amide |
| 153 | 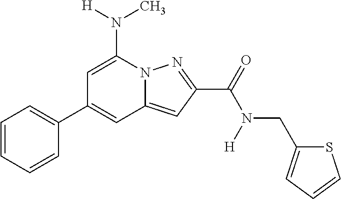 | 7-Methylamino-5-phenyl- pyrazolo[1,5-a]pyridine-2- carboxylic acid (thiophen- 2-ylmethyl)-amide |
| 154 | 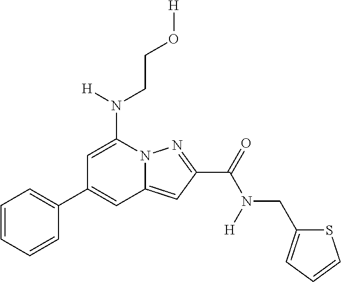 | 7-(2-Hydroxy- ethylamino)-5-phenyl- pyrazolo[1,5-a]pyridine-2- carboxylic acid (thiophen- 2-ylmethyl)-amide |
| 155 | 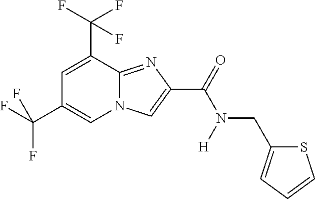 | 6,8-Bis-trifluoromethyl- imidazo[1,2-a]pyridine-2- carboxylic acid (thiophen- 2-ylmethyl)-amide |
| 156 | 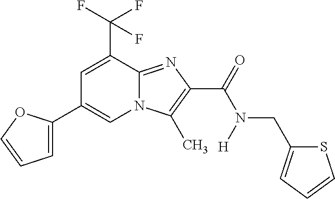 | 6-Furan-2-yl-3-methyl-8- trifluoromethyl- imidazo[1,2-a]pyridine-2- carboxylic acid (thiophen- 2-ylmethyl)-amide |
| 157 | 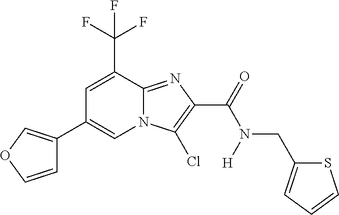 | 3-Chloro-6-furan-3-yl-8- trifluoromethyl- imidazo[1,2-a]pyridine-2- carboxylic acid (thiophen- 2-ylmethyl)-amide |
| 158 | 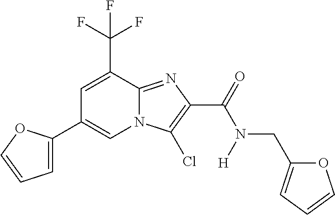 | 3-Chloro-6-furan-2-yl-8- trifluoromethyl- imidazo[1,2-a]pyridine-2- carboxylic acid (furan-2- ylmethyl)-amide |
| 159 | 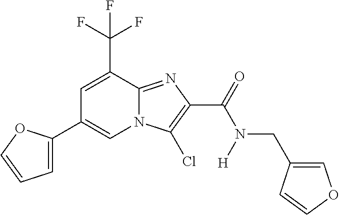 | 3-Chloro-6-furan-2-yl-8- trifluoromethyl- imidazo[1,2-a]pyridine-2- carboxylic acid (furan-3- ylmethyl)-amide |
| 160 | 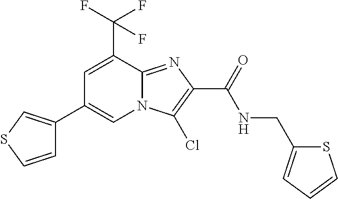 | 3-Chloro-6-thiophen-3-yl- 8-trifluoromethyl- imidazo[1,2-a]pyridine-2- carboxylic acid (thiophen- 2-ylmethyl)-amide |
| 161 | 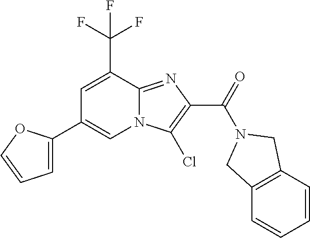 | (3-Chloro-6-furan-2-yl-8- trifluoromethyl- imidazo[1,2-a]pyridin-2- yl)-(1,3-dihydro-isoindol- 2-yl)-methanone |
| 162 | 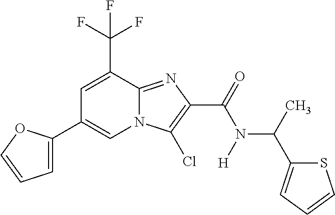 | 3-Chloro-6-furan-2-yl-8- trifluoromethyl- imidazo[1,2-a]pyridine-2- carboxylic acid (1- thiophen-2-yl-ethyl)- amide |
| 163 | 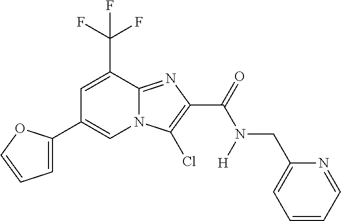 | 3-Chloro-6-furan-2-yl-8- trifluoromethyl- imidazo[1,2-a]pyridine-2- carboxylic acid (pyridin-2- ylmethyl)-amide |
| 164 | 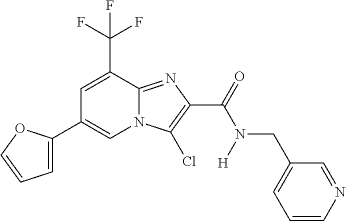 | 3-Chloro-6-furan-2-yl-8- trifluoromethyl- imidazo[1,2-a]pyridine-2- carboxylic acid (pyridin-3- ylmethyl)-amide |
| 165 | 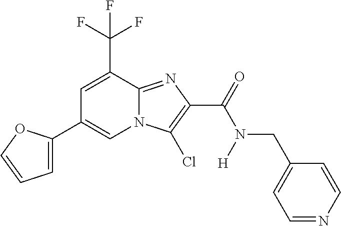 | 3-Chloro-6-furan-2-yl-8- trifluoromethyl- imidazo[1,2-a]pyridine-2- carboxylic acid (pyridin-4- ylmethyl)-amide |
| 166 | 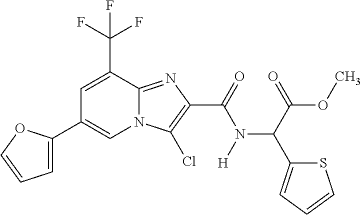 | [(3-Chloro-6-furan-2-yl-8- trifluoromethyl- imidazo[1,2-a]pyridine-2- carbonyl)-amino]- thiophen-2-yl-acetic acid methyl ester |
| 167 | 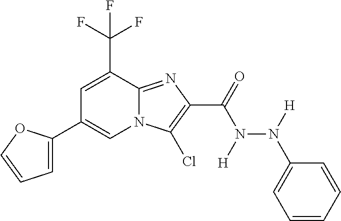 | 3-Chloro-6-furan-2-yl-8- trifluoromethyl- imidazo[1,2-a]pyridine-2- carboxylic acid N′-phenyl- hydrazide |
| 168 | 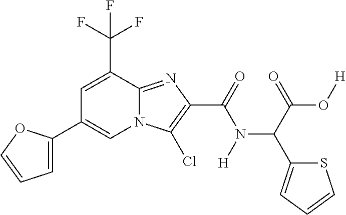 | [(3-Chloro-6-furan-2-yl-8- trifluoromethyl- imidazo[1,2-a]pyridine-2- carbonyl)-amino]- thiophen-2-yl-acetic acid |
| 169 | 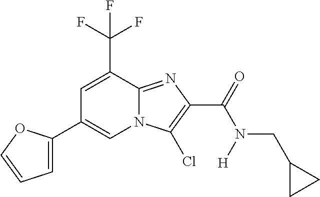 | 3-Chloro-6-furan-2-yl-8- trifluoromethyl- imidazo[1,2-a]pyridine-2- carboxylic acid cyclopropylmethyl-amide |
| 170 | 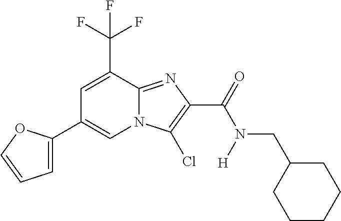 | 3-Chloro-6-furan-2-yl-8- trifluoromethyl- imidazo[1,2-a]pyridine-2- carboxylic acid cyclohexylmethyl-amide |
| 171 | 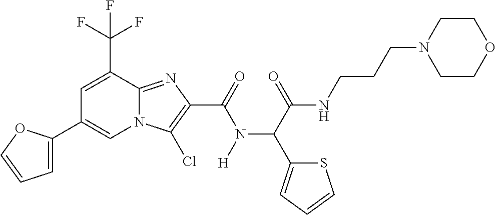 | 3-Chloro-6-furan-2-yl-8- trifluoromethyl- imidazo[1,2-a]pyridine-2- carboxylic acid [(3- morpholin-4-yl- propylcarbamoyl)- thiophen-2-yl-methyl]- amide |
| 172 | 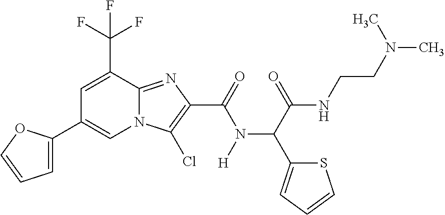 | 3-Chloro-6-furan-2-yl-8- trifluoromethyl- imidazo[1,2-a]pyridine-2- carboxylic acid [(2- dimethylamino- ethylcarbamoyl)-thiophen- 2-yl-methyl]-amide |
| 173 | 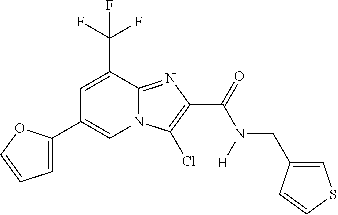 | 3-Chloro-6-furan-2-yl-8- trifluoromethyl- imidazo[1,2-a]pyridine-2- carboxylic acid (thiophen- 3-ylmethyl)-amide |
| 174 | 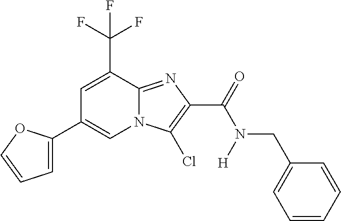 | 3-Chloro-6-furan-2-yl-8- trifluoromethyl- imidazo[1,2-a]pyridine-2- carboxylic acid benzylamide |
| 175 | 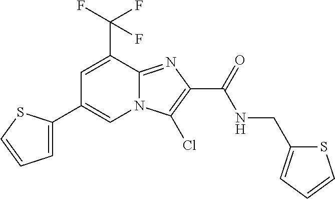 | 3-Chloro-6-thiophen-2-yl- 8-trifluoromethyl- imidazo[1,2-a]pyridine-2- carboxylic acid (thiophen- 2-ylmethyl)-amide |
| 176 | 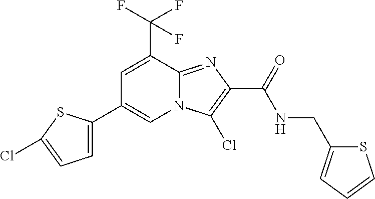 | 3-Chloro-6-(5-chloro- thiophen-2-yl)-8- trifluoromethyl- imidazo[1,2-a]pyridine-2- carboxylic acid (thiophen- 2-ylmethyl)-amide |
| 177 | 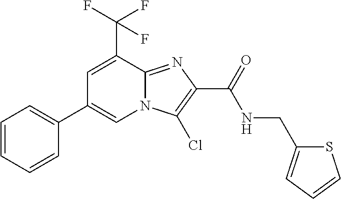 | 3-Chloro-6-phenyl-8- trifluoromethyl- imidazo[1,2-a]pyridine-2- carboxylic acid (thiophen- 2-ylmethyl)-amide |
| 178 | 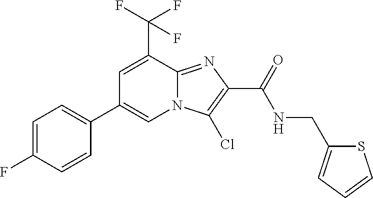 | 3-Chloro-6-(4-fluoro- phenyl)-8-trifluoromethyl- imidazo[1,2-a]pyridine-2- carboxylic acid (thiophen- 2-ylmethyl)-amide |
| 179 | 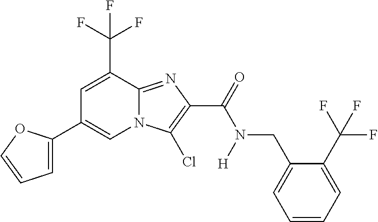 | 3-Chloro-6-furan-2-yl-8- trifluoromethyl- imidazo[1,2-a]pyridine-2- carboxylic acid 2- trifluoromethyl- benzylamide |
| 180 | 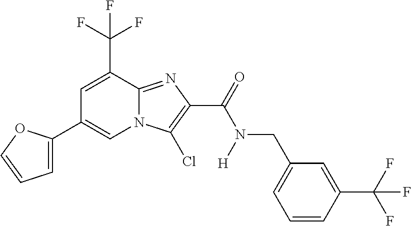 | 3-Chloro-6-furan-2-yl-8- trifluoromethyl- imidazo[1,2-a]pyridine-2- carboxylic acid 3- trifluoromethyl- benzylamide |
| 181 | 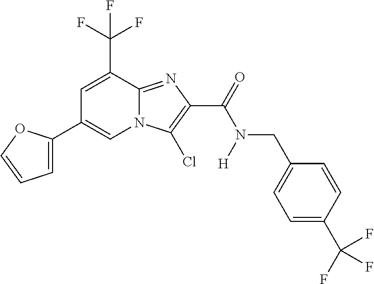 | 3-Chloro-6-furan-2-yl-8- trifluoromethyl- imidazo[1,2-a]pyridine-2- carboxylic acid 4- trifluoromethyl- benzylamide |
| 182 | 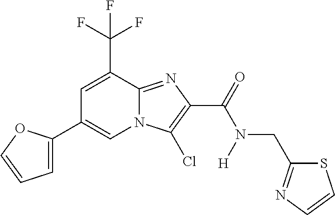 | 3-Chloro-6-furan-2-yl-8- trifluoromethyl- imidazo[1,2-a]pyridine-2- carboxylic acid (thiazol-2- ylmethyl)-amide |
| 183 | 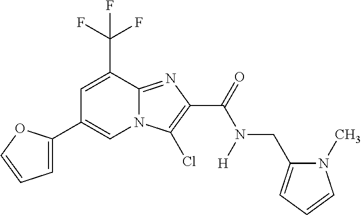 | 3-Chloro-6-furan-2-yl-8- trifluoromethyl- imidazo[1,2-a]pyridine-2- carboxylic acid (1-methyl- 1H-pyrrol-2-ylmethyl)- amide |
| 184 | 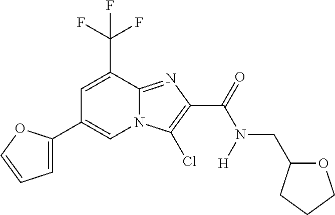 | 3-Chloro-6-furan-2-yl-8- trifluoromethyl- imidazo[1,2-a]pyridine-2- carboxylic acid (tetrahydro-furan-2- ylmethyl)-amide |
| 185 | 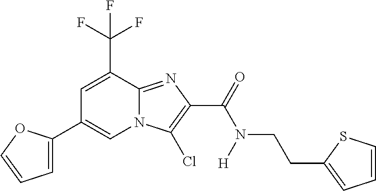 | 3-Chloro-6-furan-2-yl-8- trifluoromethyl- imidazo[1,2-a]pyridine-2- carboxylic acid (2- thiophen-2-yl-ethyl)- amide |
| 186 | 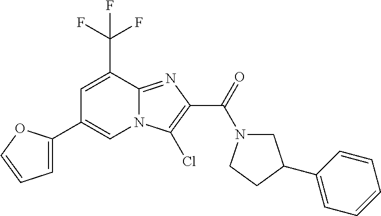 | (3-Chloro-6-furan-2-yl-8- trifluoromethyl- imidazo[1,2-a]pyridin-2- yl)-(3-phenyl-pyrrolidin-1- yl)-methanone |
| 187 | 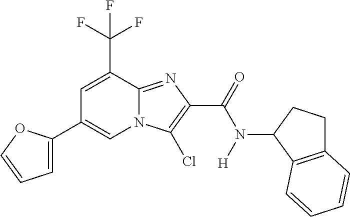 | 3-Chloro-6-furan-2-yl-8- trifluoromethyl- imidazo[1,2-a]pyridine-2- carboxylic acid indan-1- ylamide |
| 188 | 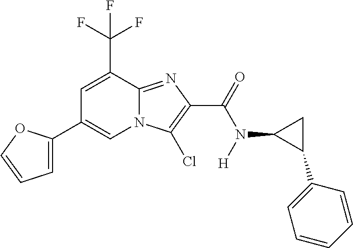 | 3-Chloro-6-furan-2-yl-8- trifluoromethyl- imidazo[1,2-a]pyridine-2- carboxylic acid (2-phenyl- cyclopropyl)-amide |
| 189 | 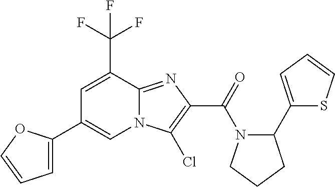 | (3-Chloro-6-furan-2-yl-8- trifluoromethyl- imidazo[1,2-a]pyridin-2- yl)-(2-thiophen-2-yl- pyrrolidin-1-yl)- methanone |
| 190 | 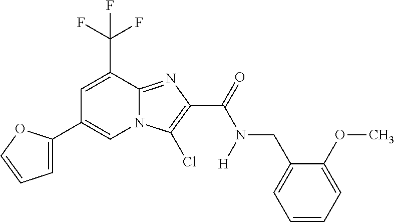 | 3-Chloro-6-furan-2-yl-8- trifluoromethyl- imidazo[1,2-a]pyridine-2- carboxylic acid 2- methoxy-benzylamide |
| 191 | 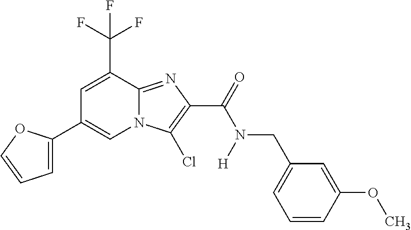 | 3-Chloro-6-furan-2-yl-8- trifluoromethyl- imidazo[1,2-a]pyridine-2- carboxylic acid 3- methoxy-benzylamide |
| 192 | 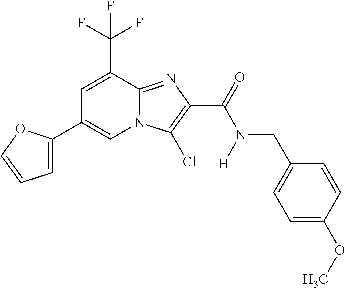 | 3-Chloro-6-furan-2-yl-8- trifluoromethyl- imidazo[1,2-a]pyridine-2- carboxylic acid 4- methoxy-benzylamide |
| 193 | 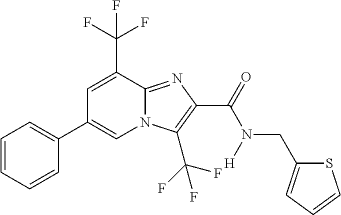 | 6-Phenyl-3,8-bis- trifluoromethyl- imidazo[1,2-a]pyridine-2- carboxylic acid (thiophen- 2-ylmethyl)-amide |
| 194 | 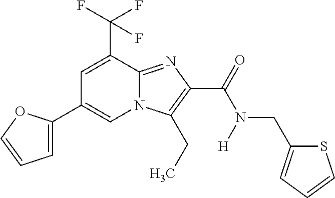 | 3-Ethyl-6-furan-2-yl-8- trifluoromethyl- imidazo[1,2-a]pyridine-2- carboxylic acid (thiophen- 2-ylmethyl)-amide |
| 195 | 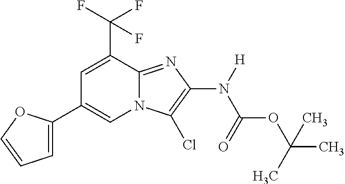 | (3-Chloro-6-furan-2-yl-8- trifluoromethyl- imidazo[1,2-a]pyridin-2- yl)-carbamic acid tert- butyl ester |
| 196 | 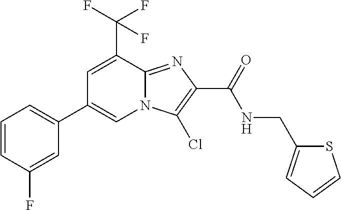 | 3-Chloro-6-(3-fluoro- phenyl)-8-trifluoromethyl- imidazo[1,2-a]pyridine-2- carboxylic acid (thiophen- 2-ylmethyl)-amide |
| 197 | 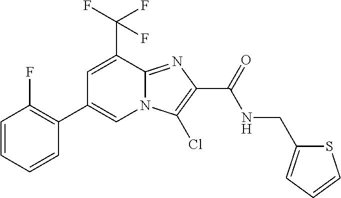 | 3-Chloro-6-(2-fluoro- phenyl)-8-trifluoromethyl- imidazo[1,2-a]pyridine-2- carboxylic acid (thiophen- 2-ylmethyl)-amide |
| 198 | 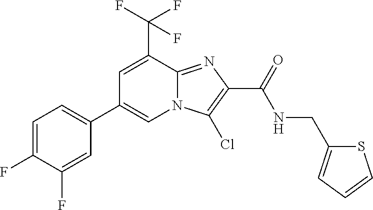 | 3-Chloro-6-(3,4-difluoro- phenyl)-8-trifluoromethyl- imidazo[1,2-a]pyridine-2- carboxylic acid (thiophen- 2-ylmethyl)-amide |
| 199 | 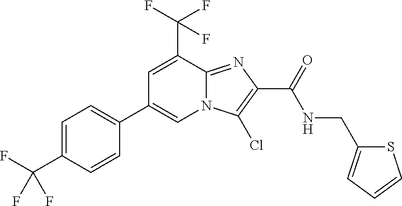 | 3-Chloro-8- trifluoromethyl-6-(4- trifluoromethyl-phenyl)- imidazo[1,2-a]pyridine-2- carboxylic acid (thiophen- 2-ylmethyl)-amide |
| 200 | 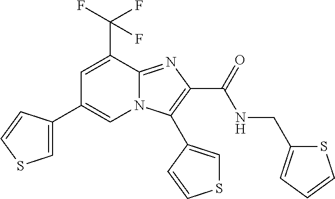 | 3,6-Di-thiophen-3-yl-8- trifluoromethyl- imidazo[1,2-a]pyridine-2- carboxylic acid (thiophen- 2-ylmethyl)-amide |
| 201 | 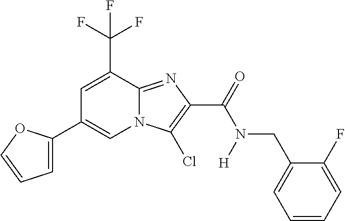 | 3-Chloro-6-furan-2-yl-8- trifluoromethyl- imidazo[1,2-a]pyridine-2- carboxylic acid 2-fluoro- benzylamide |
| 202 | 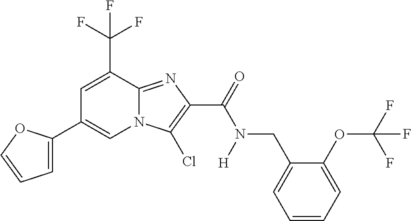 | 3-Chloro-6-furan-2-yl-8- trifluoromethyl- imidazo[1,2-a]pyridine-2- carboxylic acid 2- trifluoromethoxy- benzylamide |
| 203 | 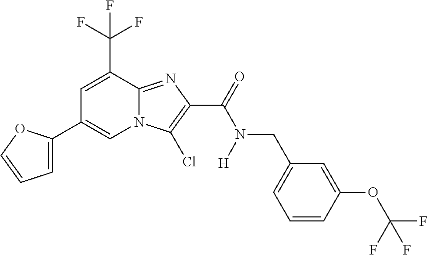 | 3-Chloro-6-furan-2-yl-8- trifluoromethyl- imidazo[1,2-a]pyridine-2- carboxylic acid 3- trifluoromethoxy- benzylamide |
| 204 | 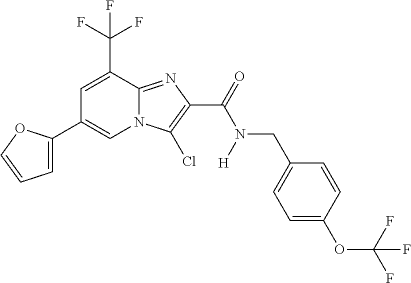 | 3-Chloro-6-furan-2-yl-8- trifluoromethyl- imidazo[1,2-a]pyridine-2- carboxylic acid 4- trifluoromethoxy- benzylamide |
| 205 | 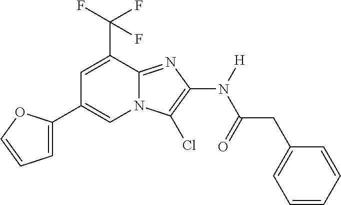 | (3-Chloro-6-furan-2-yl- 8-trifluoromethyl- imidazo[1,2-a]pyridin-2- yl)-2-phenyl-acetamide |
| 206 | 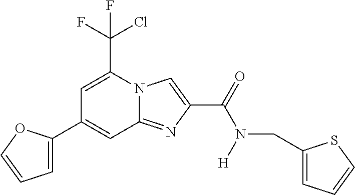 | 5-(Chloro-difluoro- methyl)-7-furan-2-yl- imidazo[1,2-a]pyridine-2- carboxylic acid (thiophen- 2-ylmethyl)-amide |
| 207 | 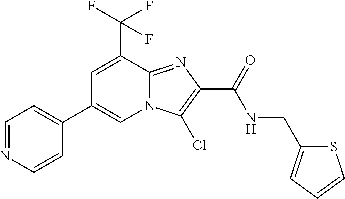 | 3-Chloro-6-pyridin-4-yl-8- trifluoromethy- imidazo[1,2-a]pyridine-2- carboxylic acid (thiophen- 2-ylmethyl)-amide |
| 208 | 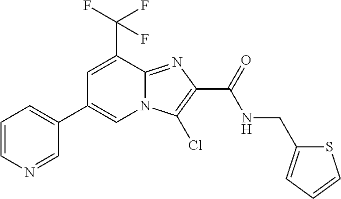 | 3-Chloro-6-pyridin-3-yl-8- trifluoromethyl- imidazo[1,2-a]pyridine-2- carboxylic acid (thiophen- 2-ylmethyl)-amide |
| 209 | 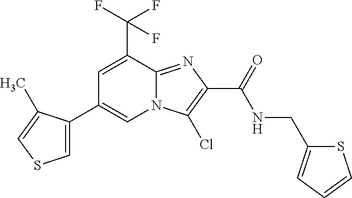 | 3-Chloro-6-(4-methyl- thiophen-3-yl)-8- trifluoromethyl- imidazo[1,2-a]pyridine-2- carboxylic acid (thiophen- 2-ylmethyl)-amide |
| 210 | 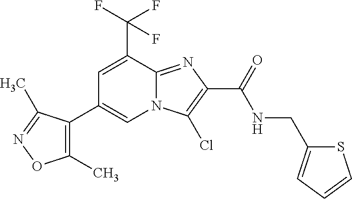 | 3-Chloro-6-(3,5-dimethyl- isoxazol-4-yl)-8- trifluoromethyl- imidazo[1,2-a]pyridine-2- carboxylic acid (thiophen- 2-ylmethyl)-amide |
| 211 | 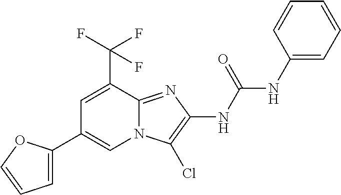 | 1-(3-Chloro-6-furan-2-yl- 8-trifluoromethyl- imidazo[1,2-a]pyridin-2- yl)-3-phenyl-urea |

TABLE 2

| Compound | | |
|---|---|---|
| Number | Structure | Compound Name |
| 212 | 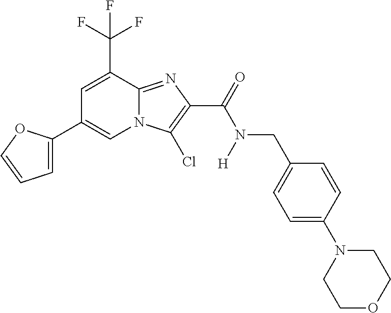 | 3-Chloro-6-furan-2-yl-8- trifluoromethyl-imidazo[1,2-a]pyridine- 2-carboxylic acid 4-morpholin-4-yl- benzylamide |
| 213 | 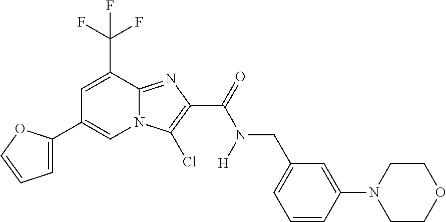 | 3-Chloro-6-furan-2-yl-8- trifluoromethyl-imidazo[1,2-a]pyridine- 2-carboxylic acid 3-morpholin-4-yl- benzylamide |
| 214 | 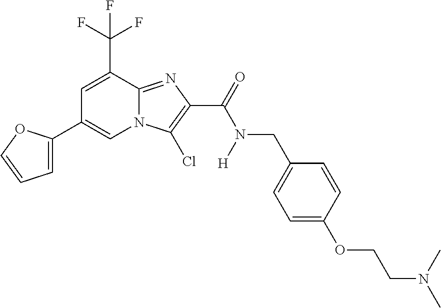 | 3-Chloro-6-furan-2-yl-8- trifluoromethyl-imidazo[1,2-a]pyridine- 2-carboxylic acid 4-(2-dimethylamino- ethoxy)-benzylamide |
| 215 | 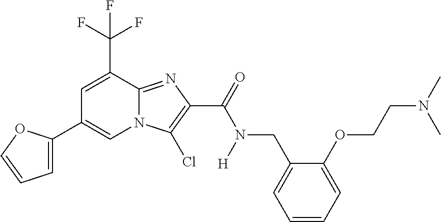 | 3-Chloro-6-furan-2-yl-8- trifluoromethyl-imidazo[1,2-a]pyridine- 2-carboxylic acid 2-(2-dimethylamino- ethoxy)-benzylamide |
| 216 | 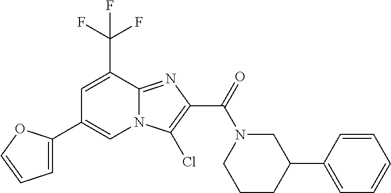 | (3-Chloro-6-furan-2-yl-8- trifluoromethyl-imidazo[1,2-a]pyridin- 2-yl)-(3-phenyl-piperidin-1-yl)- methanone |
| 217 | 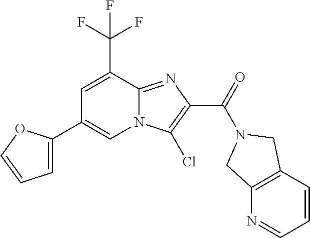 | (3-Chloro-6-furan-2-yl-8- trifluoromethyl-imidazo[1,2-a]pyridin- 2-yl)-(5,7-dihydro-pyrrolo[3,4- b]pyridin-6-yl)-methanone |
| 218 | 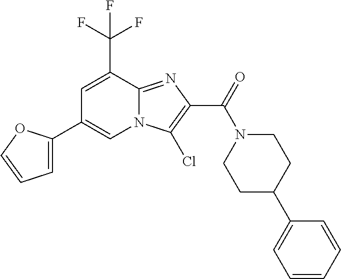 | (3-Chloro-6-furan-2-yl-8- trifluoromethyl-imidazo[1,2-a]pyridin- 2-yl)-(4-phenyl-piperidin-1-yl)- methanone |
| 219 | 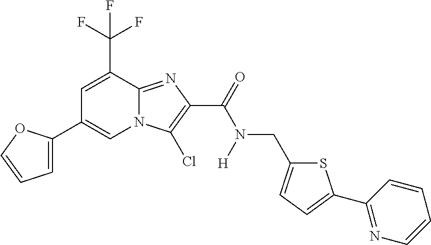 | 3-Chloro-6-furan-2-yl-8- trifluoromethyl-imidazo[1,2-a]pyridine- 2-carboxylic acid (5-pyridin-2-yl- thiophen-2-ylmethyl)-amide |
| 220 | 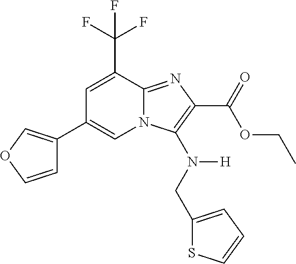 | 6-Furan-3-yl-3-[(thiophen-2-ylmethyl)- amino]-8-trifluoromethyl-imidazo[1,2- a]pyridine-2-carboxylic acid ethyl ester |
| 221 | 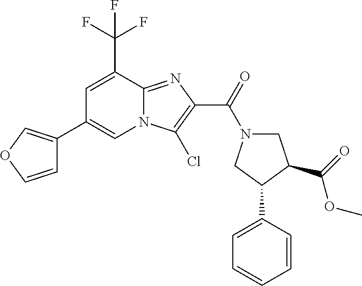 | 1-(3-Chloro-6-furan-3-yl-8- trifluoromethyl-imidazo[1,2-a]pyridine- 2-carbonyl)-4-phenyl-pyrrolidine-3- carboxylic acid methyl ester |
| 222 | 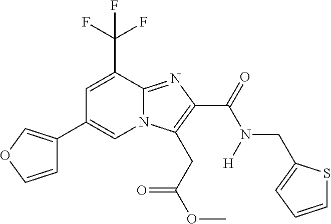 | {6-Furan-3-yl-2-[(thiophen-2- ylmethyl)-carbamoyl]-8- trifluoromethyl-imidazo[1,2-a]pyridin- 3-yl}-acetic acid methyl ester |
| 223 | 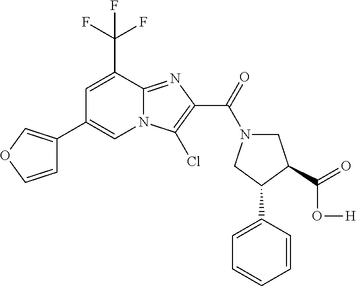 | 1-(3-Chloro-6-furan-3-yl-8- trifluoromethyl-imidazo[1,2-a]pyridine- 2-carbonyl)-4-phenyl-pyrrolidine-3- carboxylic acid |
| 224 | 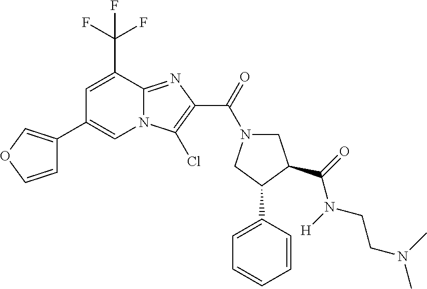 | 1-(3-Chloro-6-furan-3-yl-8- trifluoromethyl-imidazo[1,2-a]pyridine- 2-carbonyl)-4-phenyl-pyrrolidine-3- carboxylic acid (2-dimethylamino- ethyl)-amide |
| 225 | 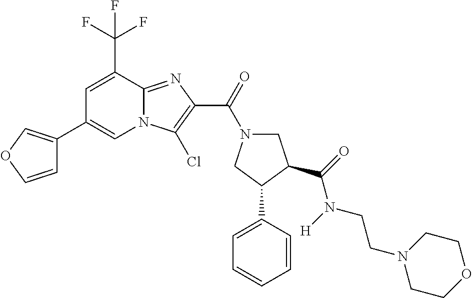 | 1-(3-Chloro-6-furan-3-yl-8- trifluoromethyl-imidazo[1,2-a]pyridine- 2-carbonyl)-4-phenyl-pyrrolidine-3- carboxylic acid (2-morpholin-4-yl- ethyl)-amide |
| 226 | 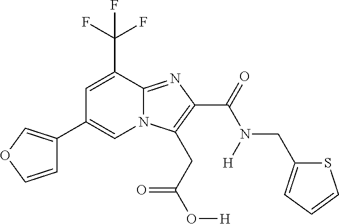 | {6-Furan-3-yl-2-[(thiophen-2- ylmethyl)-carbamoyl]-8- trifluoromethyl-imidazo[1,2-a]pyridin- 3-yl}-acetic acid |
| 227 | 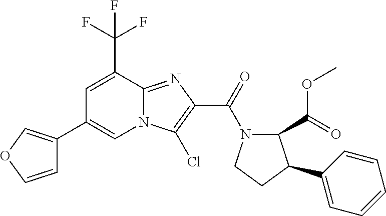 | 1-(3-Chloro-6-furan-3-yl-8- trifluoromethyl-imidazo[1,2-a]pyridine- 2-carbonyl)-3-phenyl-pyrrolidine-2- carboxylic acid methyl ester |
| 228 | 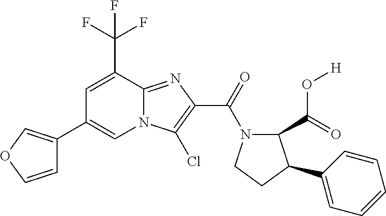 | 1-(3-Chloro-6-furan-3-yl-8- trifluoromethyl-imidazo[1,2-a]pyridine- 2-carbonyl)-3-phenyl-pyrrolidine-2- carboxylic acid |
| 229 | 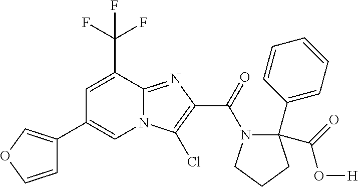 | 1-(3-Chloro-6-furan-3-yl-8- trifluoromethyl-imidazo[1,2-a]pyridine- 2-carbonyl)-2-phenyl-pyrrolidine-2- carboxylic acid |
| 230 | 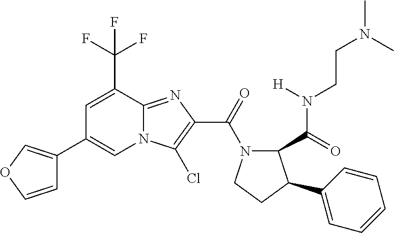 | 1-(3-Chloro-6-furan-3-yl-8- trifluoromethyl-imidazo[1,2-a]pyridine- 2-carbonyl)-3-phenyl-pyrrolidine-2- carboxylic acid (2-dimethylamino- ethyl)-amide |
| 231 | 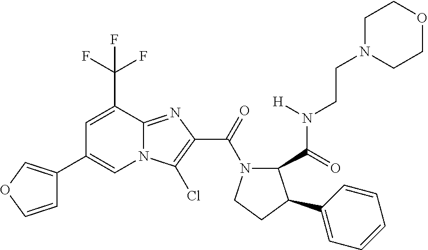 | 1-(3-Chloro-6-furan-3-yl-8- trifluoromethyl-imidazo[1,2-a]pyridine- 2-carbonyl)-3-phenyl-pyrrolidine-2- carboxylic acid (2-morpholin-4-yl- ethyl)-amide |
| 232 | 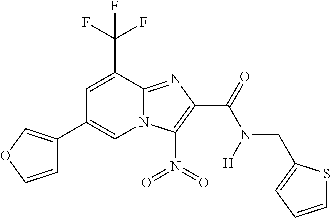 | 6-Furan-3-yl-3-nitro-8-trifluoromethyl- imidazo[1,2-a]pyridine-2-carboxylic acid (thiophen-2-ylmethyl)-amide |
| 233 | 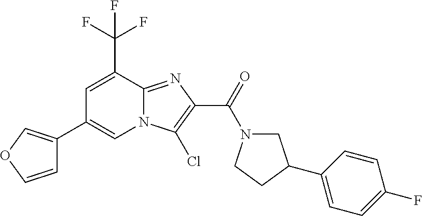 | (3-Chloro-6-furan-3-yl-8- trifluoromethyl-imidazo[1,2-a]pyridin- 2-yl)-[3-(4-fluoro-phenyl)-pyrrolidin-1- yl]-methanone |
| 234 | 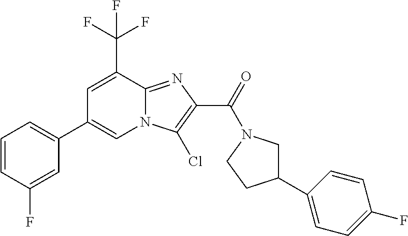 | [3-Chloro-6-(3-fluoro-phenyl)-8- trifluoromethyl-imidazo[1,2-a]pyridin- 2-yl]-[3-(4-fluoro-phenyl)-pyrrolidin-1- yl]-methanone |
| 235 | 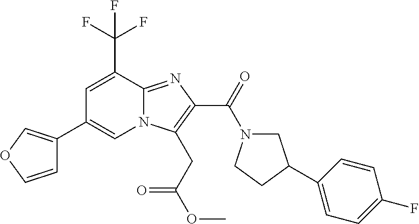 | {2-[3-(4-Fluoro-phenyl)-pyrrolidine-1- carbonyl]-6-furan-3-yl-8- trifluoromethyl-imidazo[1,2-a]pyridin- 3-yl}-acetic acid methyl ester |
| 236 | 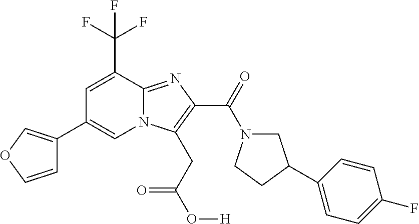 | {2-[3-(4-Fluoro-phenyl)-pyrrolidine-1- carbonyl]-6-furan-3-yl-8- trifluoromethyl-imidazo[1,2-a]pyridin- 3-yl}-acetic acid |
| 237 | 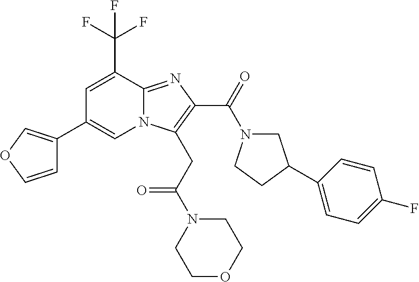 | 2-{2-[3-(4-Fluoro-phenyl)-pyrrolidine- 1-carbonyl]-6-furan-3-yl-8- trifluoromethyl-imidazo[1,2-a]pyridin- 3-yl}-1-morpholin-4-yl-ethanone |
| 238 | 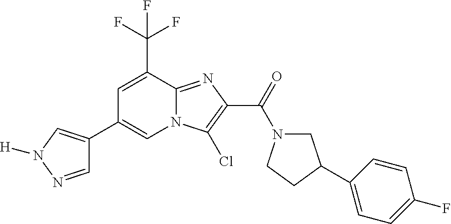 | [3-Chloro-6-(1H-pyrazol-4-yl)-8- trifluoromethyl-imidazo[1,2-a]pyridin- 2-yl]-[3-(4-fluoro-phenyl)-pyrrolidin-1- yl]-methanone |
| 239 | 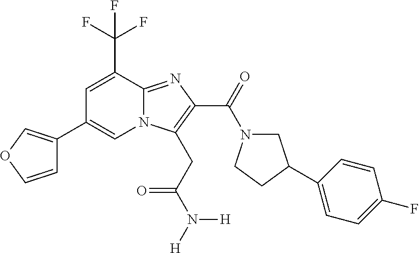 | 2-{2-[3-(4-Fluoro-phenyl)-pyrrolidine- 1-carbonyl]-6-furan-3-yl-8- trifluoromethyl-imidazo[1,2-a]pyridin- 3-yl}-acetamide |
| 240 | 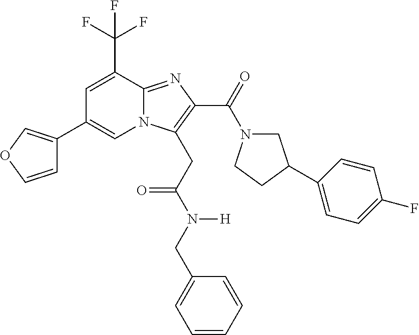 | N-Benzyl-2-{2-[3-(4-fluoro-phenyl)- pyrrolidine-1-carbonyl]-6-furan-3-yl-8- trifluoromethyl-imidazo[1,2-a]pyridin- 3-yl}-acetamide |
| 241 | 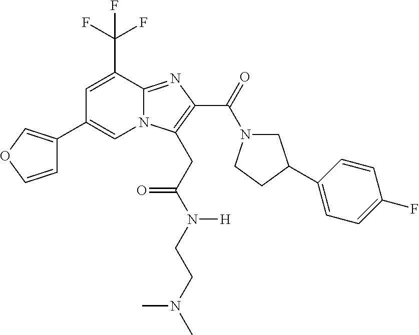 | N-(2-Dimethylamino-ethyl)-2-{2-[3-(4- fluoro-phenyl)-pyrrolidine-1-carbonyl]- 6-furan-3-yl-8-trifluoromethyl- imidazo[1,2-a]pyridin-3-yl}-acetamide |
| 242 | 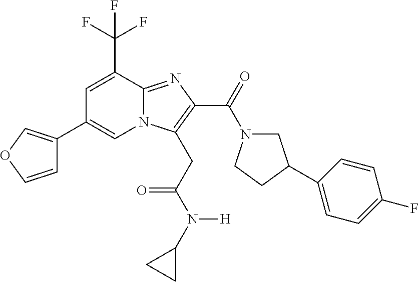 | N-Cyclopropyl-2-{2-[3-(4-fluoro- phenyl)-pyrrolidine-1-carbonyl]-6- furan-3-yl-8-trifluoromethyl- imidazo[1,2-a]pyridin-3-yl}-acetamide |
| 243 | 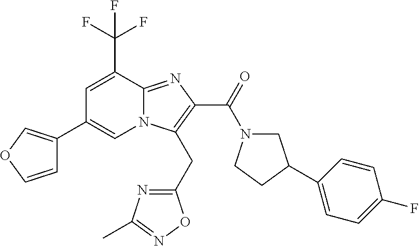 | [3-(4-Fluoro-phenyl)-pyrrolidin-1-yl]- [6-furan-3-yl-3-(3-methyl- [1,2,4]oxadiazol-5-ylmethyl)-8- trifluoromethyl-imidazo[1,2-a]pyridin- 2-yl]-methanone |
| 244 | 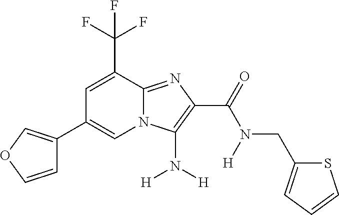 | 3-Amino-6-furan-3-yl-8- trifluoromethyl-imidazo[1,2-a]pyridine- 2-carboxylic acid (thiophen-2- ylmethyl)-amide |
| 245 | 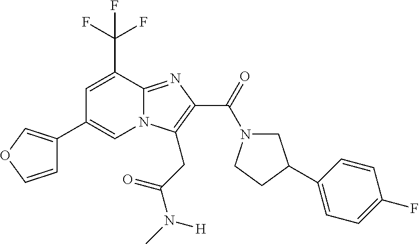 | 2-{2-[3-(4-Fluoro-phenyl)-pyrrolidine- 1-carbonyl]-6-furan-3-yl-8- trifluoromethyl-imidazo[1,2-a]pyridin- 3-yl}-N-methyl-acetamide |
| 246 | 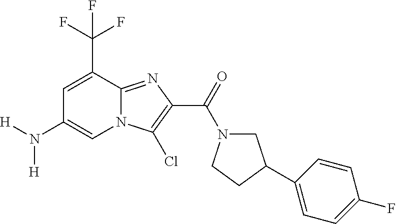 | (6-Amino-3-chloro-8-trifluoromethyl- imidazo[1,2-a]pyridin-2-yl)-[3-(4- fluoro-phenyl)-pyrrolidin-1-yl]- methanone |
| 247 | 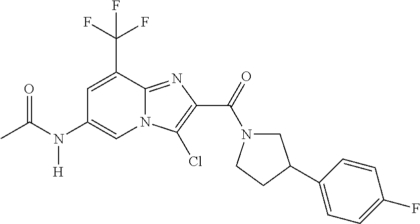 | N-{3-Chloro-2-[3-(4-fluoro-phenyl)- pyrrolidine-1-carbonyl]-8- trifluoromethyl-imidazo[1,2-a]pyridin- 6-yl}-acetamide |
| 248 | 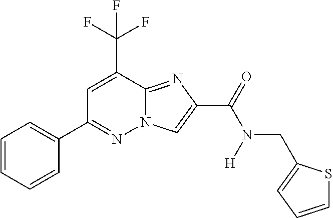 | 6-Phenyl-8-trifluoromethyl- imidazo[1,2-b]pyridazine-2-carboxylic acid (thiophen-2-ylmethyl)-amide |
| 249 | 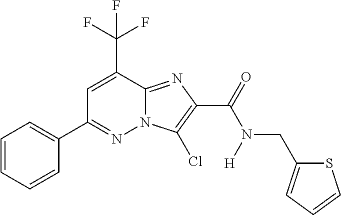 | 3-Chloro-6-phenyl-8-trifluoromethyl- imidazo[1,2-b]pyridazine-2-carboxylic acid (thiophen-2-ylmethyl)-amide |
| 250 | 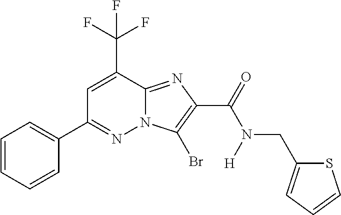 | 3-Bromo-6-phenyl-8-trifluoromethyl- imidazo[1,2-a]pyridazine-2-carboxylic acid (thiophen-2-ylmethyl)-amide |
| 251 | 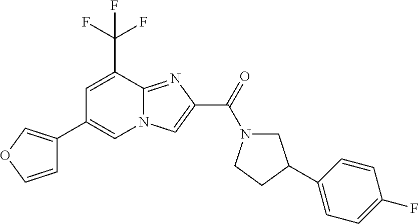 | [3-(4-Fluoro-phenyl)-pyrrolidin-1-yl]- (6-furan-3-yl-8-trifluoromethyl- imidazo[1,2-a]pyridin-2-yl)-methanone |
| 252 | 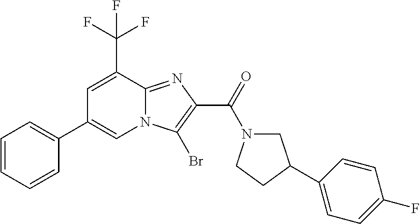 | (3-Bromo-6-phenyl-8-trifluoromethyl- imidazo[1,2-b]pyridazin-2-yl)-[3-(4- fluoro-phenyl)-pyrrolidin-1-yl]- methanone |
| 253 | 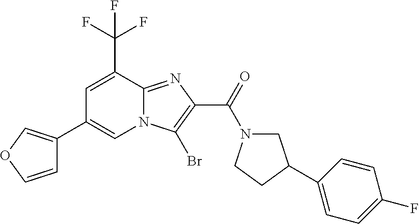 | (3-Bromo-6-furan-3-yl-8- trifluoromethyl-imidazo[1,2-a]pyridin- 2-yl)-[3-(4-fluoro-phenyl)-pyrrolidin-1- yl]-methanone |
| 254 | 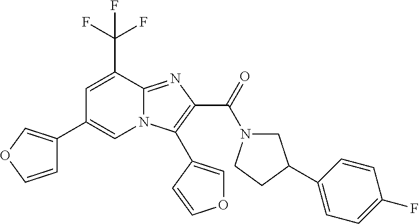 | (3,6-Di-furan-3-yl-8-trifluoromethyl- imidazo[1,2-a]pyridin-2-yl)-[3-(4- fluoro-phenyl)-pyrrolidin-1-yl]- methanone |
| 255 | 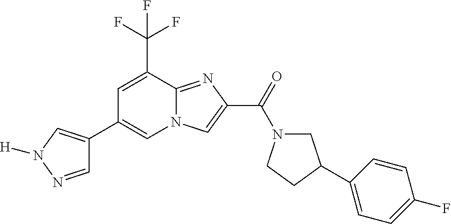 | [3-(4-Fluoro-phenyl)-pyrrolidin-1-yl]- [6-(1H-pyrazol-4-yl)-8-trifluoromethyl- imidazo[1,2-a]pyridin-2-yl]-methanone |
| 256 | 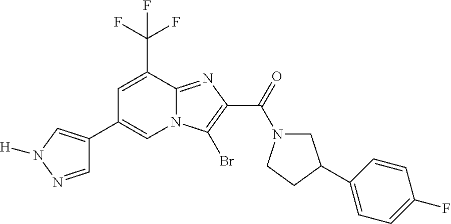 | [3-Bromo-6-(1H-pyrazol-4-yl)-8- trifluoromethyl-imidazo[1,2-a]pyridin- 2-yl]-[3-(4-fluoro-phenyl)-pyrrolidin-1- yl]-methanone |
| 257 | 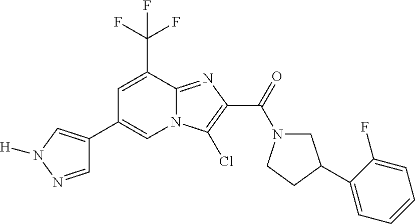 | [3-Chloro-6-(1H-pyrazol-4-yl)-8- trifluoromethyl-imidazo[1,2-a]pyridin- 2-yl]-[3-(2-fluoro-phenyl)-pyrrolidin-1- yl]-methanone |
| 258 | 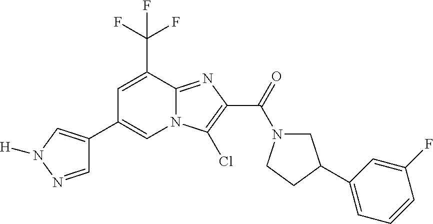 | [3-Chloro-6-(1H-pyrazol-4-yl)-8- trifluoromethyl-imidazo[1,2-a]pyridin- 2-yl]-[3-(3-fluoro-phenyl)-pyrrolidin-1- yl]-methanone |
| 259 | 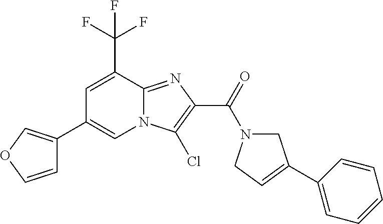 | (3-Chloro-6-furan-3-yl-8- trifluoromethyl-imidazo[1,2-a]pyridin- 2-yl)-(3-phenyl-2,5-dihydro-pyrrol-1- yl)-methanone |
| 260 | 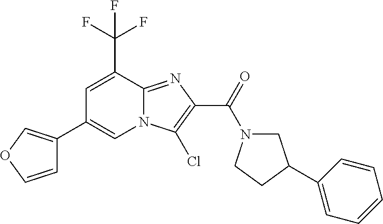 | (3-Chloro-6-furan-3-yl-8- trifluoromethyl-imidazo[1,2-a]pyridin- 2-yl)-(3-phenyl-pyrrolidin-1-yl)- methanone |
| 261 | 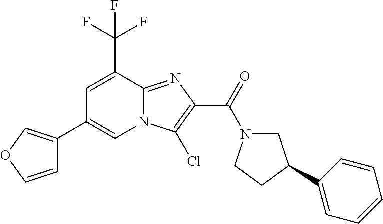 | (3-Chloro-6-furan-3-yl-8- trifluoromethyl-imidazo[1,2-a]pyridin- 2-yl)-(3-(R)-phenyl-pyrrolidin-1-yl)- methanone |
| 262 | 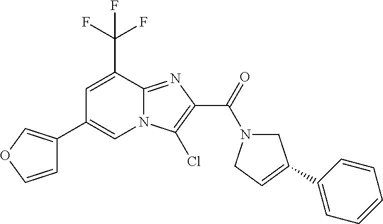 | (3-Chloro-6-furan-3-yl-8- trifluoromethyl-imidazo[1,2-a]pyridin- 2-yl)-(3-(S)-phenyl-pyrrolidin-1-yl)- methanone |
| 263 | 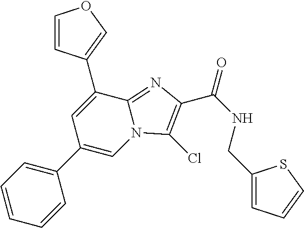 | 3-Chloro-8-furan-3-yl-6-phenyl- imidazo[1,2-a]pyridin-2-carboxylic acid (thiophen-2-ylmethyl)-amide |
| 264 | 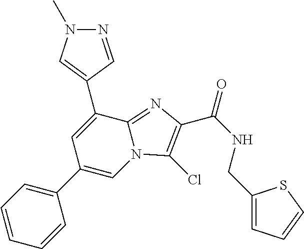 | 3-Chloro-8-(1-methyl-1H-pyrazol-4- yl)-6-phenyl-imidazo[1,2-a]pyridine-2- carboxylic acid (thiophen-2-ylmethyl)- amide |
| 265 | 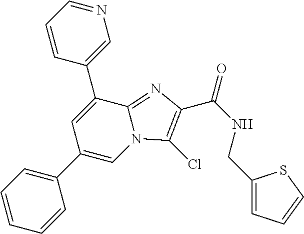 | 3-Chloro-6-phenyl-8-pyridin-3-yl- imidazo[1,2-a]pyridine-2-carboxylic acid (thiophen-2-ylmethyl)-amide |
| 266 | 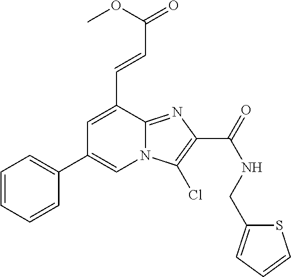 | 3-{3-Chloro-6-phenyl-2-[(thiophen-2- ylmethyl)-carbamoyl]-imidazo[1,2- a]pyridin-8-yl}-acrylic acid methyl ester |
| 267 | 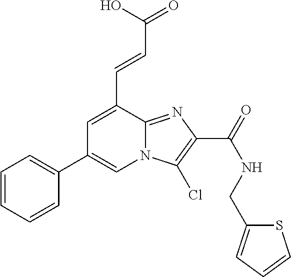 | 3-{3-Chloro-6-phenyl-2-[(thiophen-2- ylmethyl)-carbamoyl]-imidazo[1,2- a]pyridin-8-yl}-acrylic acid |
| 268 | 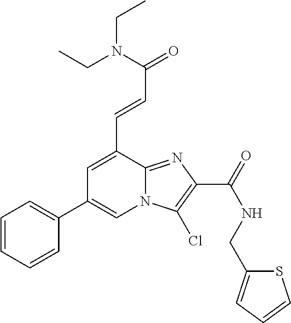 | 3-Chloro-8-(2-diethylcarbamoyl-vinyl)- 6-phenyl-imidazo[1,2-a]pyridine-2- carboxylic acid (thiophen-2-ylmethyl)- amide |
| 269 | 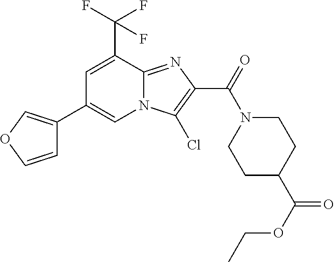 | 1-(3-Chloro-6-furan-3-yl-8- trifluoromethyl-imidazo[1,2-a]pyridine- 2-carbonyl)-piperidine-4-carboxylic acid ethyl ester |
| 270 | 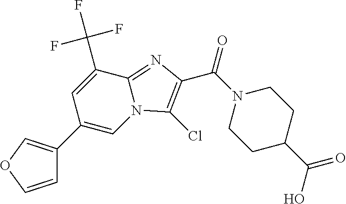 | 1-(3-Chloro-6-furan-3-yl-8- trifluoromethyl-imidazo[1,2-a]pyridine- 2-carbonyl)-piperidine-4-carboxylic acid |
| 271 | 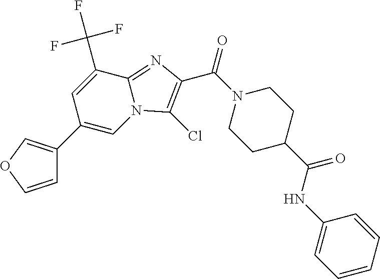 | 1-(3-Chloro-6-furan-3-yl-8- trifluoromethyl-imidazo[1,2-a]pyridine- 2-carbonyl)-piperidine-4-carboxylic acid phenylamide |
| 272 | 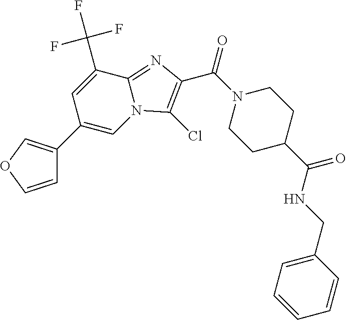 | 1-(3-Chloro-6-furan-3-yl-8- trifluoromethyl-imidazo[1,2-a]pyridine- 2-carbonyl)-piperidine-4-carboxylic acid benzylamide |
| 273 | 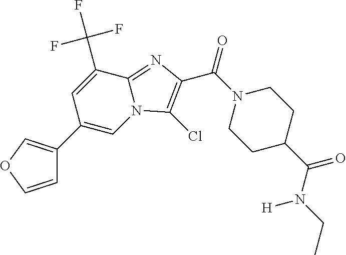 | 1-(3-Chloro-6-furan-3-yl-8- trifluoromethyl-imidazo[1,2-a]pyridine- 2-carbonyl)-piperidine-4-carboxylic acid ethylamide |
| 274 | 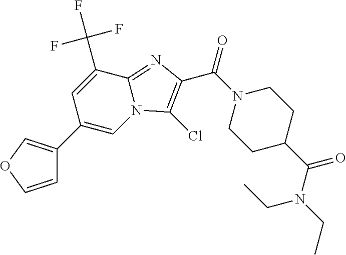 | 1-(3-Chloro-6-furan-3-yl-8- trifluoromethyl-imidazo[1,2-a]pyridine- 2-carobnyl)-piperidine-4-carboxylic acid diethylamide |
| 275 | 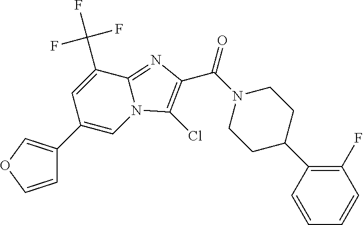 | (3-Chloro-6-furan-3-yl-8- trifluoromethyl-imidazo[1,2-a]pyridin- 2-yl)-[4-(2-fluoro-phenyl)-piperidin-1- yl]-methanone |
| 276 | 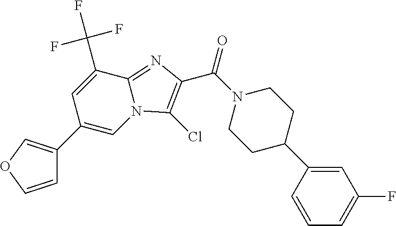 | (3-Chloro-6-furan-3-yl-8- trifluoromethyl-imidazo[1,2-a]pyridin- 2-yl)-[4-(3-fluoro-phenyl)-piperidin-1- yl]-methanone |
| 277 | 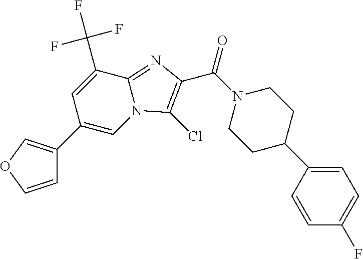 | (3-Chloro-6-furan-3-yl-8- trifluoromethyl-imidazo[1,2-a]pyridin- 2-yl)-[4-(4-fluoro-phenyl)-piperidin-1- yl]-methanone |
| 278 | 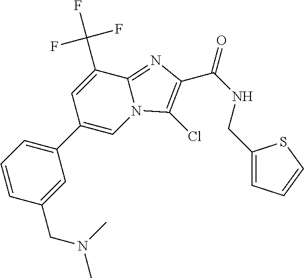 | 3-Chloro-6-(3-dimethylaminomethyl- phenyl)-8-trifluoromethyl-imidazo[1,2- a]pyridine-2-carboxylic acid (thiophen- 2-ylmethyl)-amide |
| 279 | 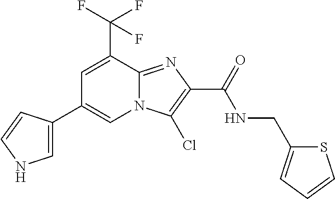 | 3-Chloro-6-(1H-pyrrol-3-yl)-8- trifluoromethyl-imidazo[1,2-a]pyridine- 2-carboxylic acid (thiophen-2- ylmethyl)-amide |
| 280 | 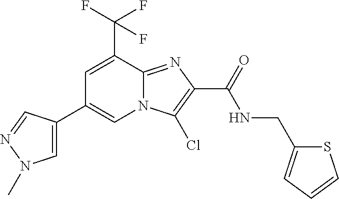 | 3-Chloro-6-(1-methyl-1H-pyrazol-4- yl)-8-trifluoromethyl-imidazo[1,2- a]pyridine-2-carboxylic acid (thiophen- 2-ylmethyl)-amide |
| 281 | 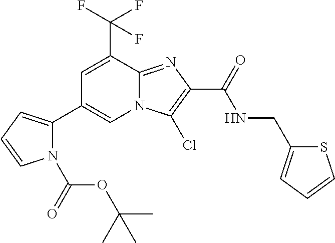 | 2-{3-Chloro-2-[(thiophen-2-ylmethyl)- carbamoyl]-8-trifluoromethyl- imidazo[1,2-a]pyridin-6-yl}-pyrrole-1- carboxylic acid tert-butyl ester |
| 282 | 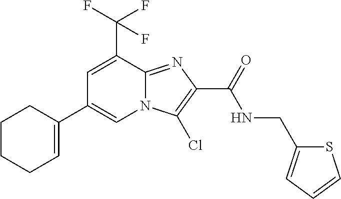 | 3-Chloro-6-cyclohex-1-enyl-8- trilfuoromethyl-imidazo[1,2-a]pyridine- 2-carboxylic acid (thiophen-2- ylmethyl)-amide |
| 283 | 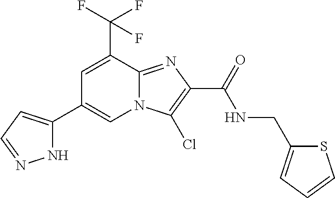 | 3-Chloro-6-(2H-pyrazol-3-yl)-8- trifluoromethyl-imidazo[1,2-a]pyridine- 2-carboxylic acid (thiophen-2- ylmethyl)-amide |
| 284 | 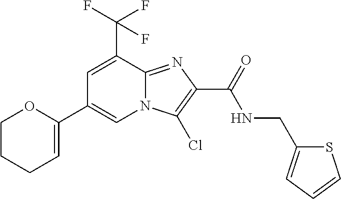 | 3-Chloro-6-(5,6-dihydro-4H-pyran-2- yl)-8-trifluoromethyl-imidazo[1,2- a]pyridine-2-carboxylic acid (thiophen- 2-ylmethyl)-amide |
| 285 | 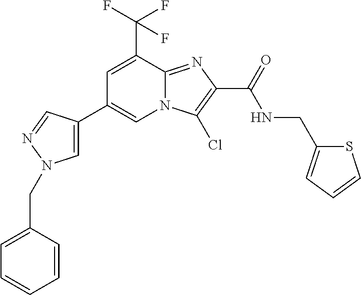 | 6-(1-Benzyl-1H-pyrazol-4-yl)-3-chloro- 8-trifluoromethyl-imidazo[1,2- a]pyridine-2-carboxylic acid (thiophen- 2-ylmethyl)-amide |
| 286 | 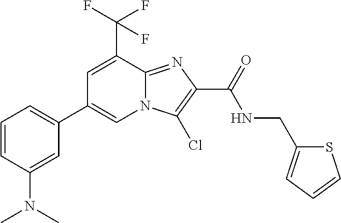 | 3-Chloro-6-(3-dimethylamino-phenyl)- 8-trifluoromethyl-imidazo[1,2- a]pyridine-2-carboxylic acid (thiophen- 2-ylmethyl)-amide |
| 287 | 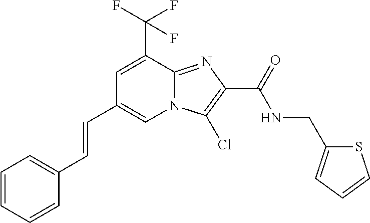 | 3-Chloro-6-styryl-8-trifluoromethyl- imidazo[1,2-a]pyridine-2-carboxylic acid (thiophen-2-ylmethyl)-amide |
| 288 | 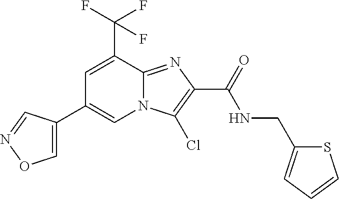 | 3-Chloro-6-isoxazol-4-yl-8- trifluoromethyl-imidazo[1,2-a]pyridine- 2-carboxylic acid (thiophen-2- ylmethyl)-amide |
| 289 | 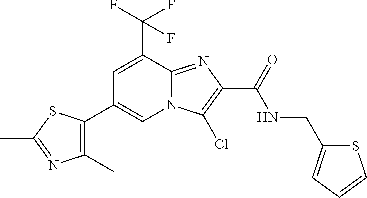 | 3-Chloro-6-(2,4-dimethyl-thiazol-5-yl)- 8-trifluoromethyl-imidazo[1,2- a]pyridine-2-carboxylic acid (thiophen- 2-ylmethyl)-amide |
| 290 | 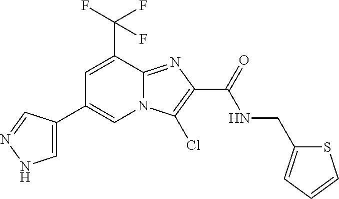 | 3-Chloro-6-(1H-pyrazol-4-yl)-8- trifluoromethyl-imidazo[1,2-a]pyridine- 2-carboxylic acid (thiophen-2- ylmethyl)-amide |
| 291 | 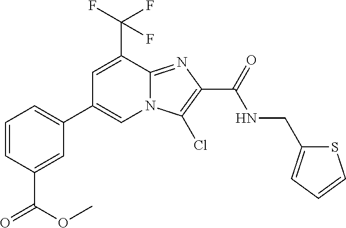 | 3-{3-Chloro-2-[(thiophen-2-ylmethyl)- carbamoyl]-8-trifluoromethyl- imidazo[1,2-a]pyridin-6-yl}-benzoic acid methyl ester |
| 292 | 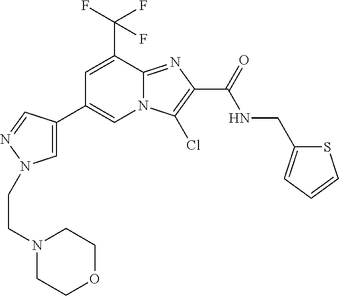 | 3-Chloro-6-[1-(2-morpholin-4-yl- ethyl)-1H-pyrazol-4-yl]-8- trifluoromethyl-imdiazo[1,2-a]pyridine- 2-carboxylic acid (thiophen-2- ylmethyl)-amide |
| 293 | 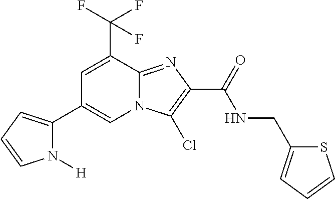 | 3-Chloro-6-(1H-pyrrol-2-yl)-8- trifluoromethyl-imidazo[1,2-a]pyridine- 2-carboxylic acid (thiophen-2- ylmethyl)-amide |
| 294 | 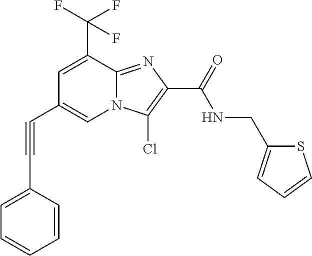 | 3-Chloro-6-phenylethynyl-8- trifluoromethyl-imidazo[1,2-a]pyridin- 2-carboxylic acid (thiophen-2- ylmethyl)-amide |
| 295 | 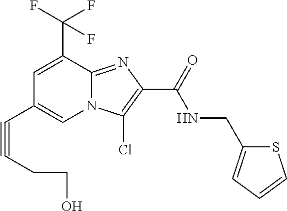 | 3-Chloro-6-(4-hydroxy-but-1-ynyl)-8- trifluoromethyl-imidazo[1,2-a]pyridine- 2-carboxylic acid (thiophen-2- ylmethyl)-amide |
| 296 | 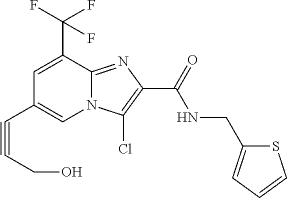 | 3-Chloro-6-(3-hydroxy-prop-1-ynyl)-8- trifluoromethyl-imidazo[1,2-a]pyridine- 2-carboxylic acid (thiophen-2- ylmethyl)-amide |
| 297 | 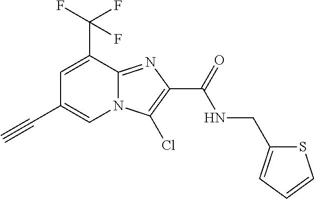 | 3-Chloro-6-ethynyl-8-trifluoromethyl- imidazo[1,2-a]pyridine-2-carboxylic acid (thiophen-2-ylmethyl)-amide |
| 298 | 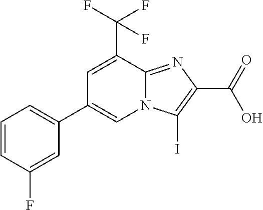 | 6-(3-Fluoro-phenyl)-3-iodo-8- trifluoromethyl-imidazo[1,2-a]pyridine- 2-carboxylic acid |
| 299 | 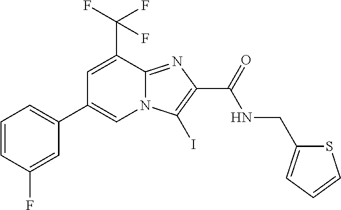 | 6-(3-Fluoro-phenyl)-3-iodo-8- trifluoromethyl-imidazo[1,2-a]pyridine- 2-carboxylic acid (thiophen-2- ylmethyl)-amide |
| 300 | 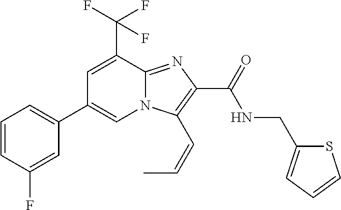 | 6-(3-Fluoro-phenyl)-3-propenyl-8- trifluoromethyl-imidazo[1,2-a]pyridine- 2-carboxylic acid (thiophen-2- ylmethyl)-amide |
| 301 | 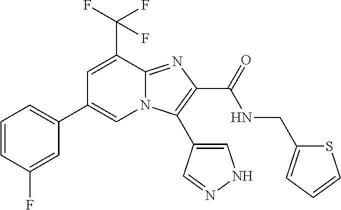 | 6-(3-Fluoro-phenyl)-3-(1H-pyrazol-4- yl)-8-trifluoromethyl-imidazo[1,2- a]pyridine-2-carboxylic acid (thiophen- 2-ylmethyl)-amide |
| 302 | 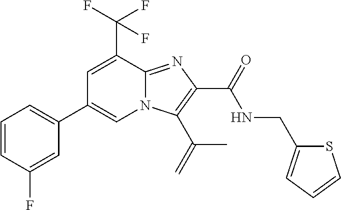 | 6-(3-Fluoro-phenyl)-3-isopropenyl-8- trifluoromethyl-imidazo[1,2-a]pyridine- 2-carboxylic acid (thiophen-2- ylmethyl)-amide |
| 303 | 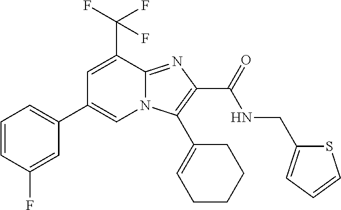 | 3-Cyclohex-1-enyl-6-(3-fluoro-phenyl)- 8-trifluoromethyl-imidazo[1,2- a]pyridine-2-carboxylic acid (thiophen- 2-ylmethyl)-amide |
| 304 | 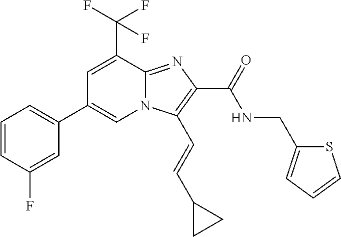 | 3-(2-Cyclopropyl-vinyl)-6-(3-fluoro- phenyl)-8-trifluoromethyl-imidazo[1,2- a]pyridine-2-carboxylic acid (thiophen- 2-ylmethyl)-amide |
| 305 | 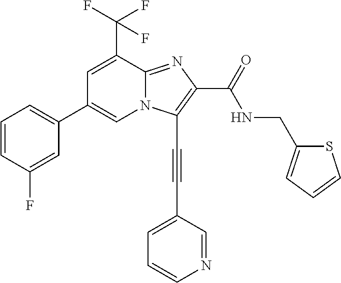 | 6-(3-Fluoro-phenyl)-3-pyridin-3- ylethynyl-8-trifluoromethyl- imidazo[1,2-a]pyridine-2-carboxylic acid (thiophen-2-ylmethyl)-amide |
| 306 | 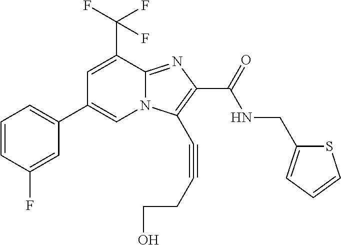 | 6-(3-Fluoro-phenyl)-3-(4-hydroxy-but- 1-ynyl)-8-trifluoromethyl-imidazo[1,2- a]pyridine-2-carboxylic acid (thiophen- 2-ylmethyl)-amide |
| 307 | 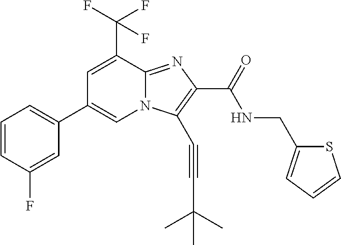 | 3-(3,3-Dimethyl-but-1-ynyl)-6-(3- fluoro-phenyl)-8-trifluoromethyl- imidazo[1,2-a]pyridine-2-carboxylic acid (thiophen-2-ylmethyl)-amide |
| 308 | 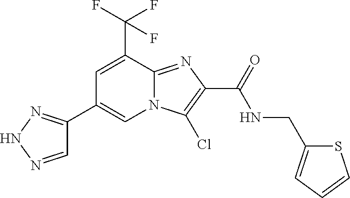 | 3-Chloro-6-(2H-[1,2,3]triazol-4-yl)-8- trifluoromethyl-imidazo[1,2-a]pyridine- 2-carboxylic acid (thiophen-2- ylmethyl)-amide |
| 309 | 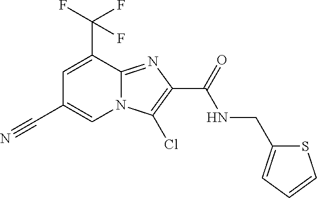 | 3-Chloro-6-cyano-8-trifluoromethyl- imidazo[1,2-a]pyridine-2-carboxylic acid (thiophen-2-ylmethyl)-amide |
| 310 | 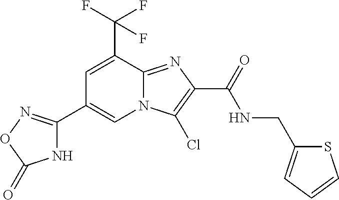 | 3-Chloro-6-(5-oxo-4,5-dihydro- [1,2,4]oxadiazol-3-yl)-8- trifluoromethyl-imidazo[1,2-a]pyridine- 2-carboxylic acid (thiophen-2- ylmethyl)-amide |
| 311 | 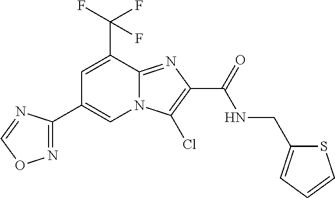 | 3-Chloro-6-[1,2,4]oxadiazol-3-yl-8- trifluoromethyl-imidazo[1,2-a]pyridine- 2-carboxylic acid (thiophen-2- ylmethyl)-amide |
| 312 | 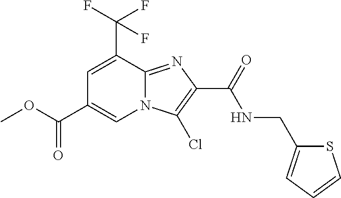 | 3-Chloro-2-[(thiophen-2-ylmethyl)- carbamoyl]-8-trifluoromethyl- imidazo[1,2-a]pyridine-6-carboxylic acid methyl ester |
| 313 | 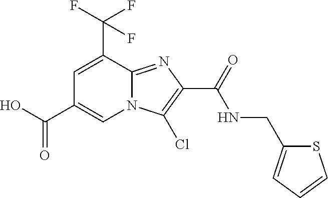 | 3-Chloro-2-[(thiophen-2-ylmethyl)- carbamoyl]-8-trifluoromethyl- imidazo[1,2-a]pyridine-6-carboxylic acid |
| 314 | 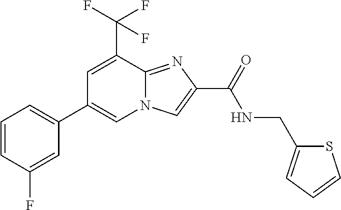 | 6-(3-Fluoro-phenyl)-8-trifluoromethyl- imidazo[1,2-a]pyridine-2-carboxylic acid (thiophen-2-ylmethyl)-amide |
| 315 | 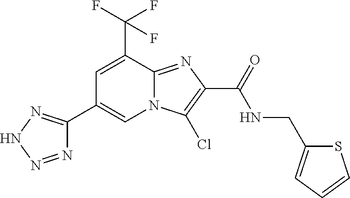 | 3-Chloro-6-(2H-tetrazol-5-yl)-8- trifluoromethyl-imidazo[1,2-a]pyridine- 2-carboxylic acid (thiophen-2- ylmethyl)-amide |
| 316 | 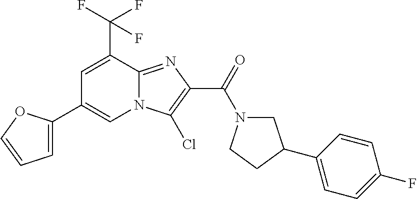 | (3-Chloro-6-furan-2-yl-8- trifluoromethyl-imidazo[1,2-a]pyridin- 2-yl)-[3-(4-fluoro-phenyl)-pyrrolidin-1- yl]-methanone |
| 317 | 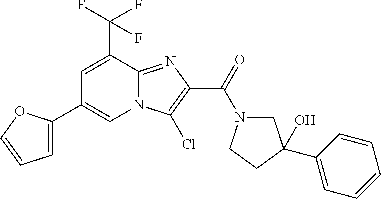 | (3-Chloro-6-furan-2-yl-8- trifluoromethyl-imidazo[1,2-a]pyridin- 2-yl)-(3-hydroxy-3-phenyl-pyrrolidin- 1-yl)-methanone |
| 318 | 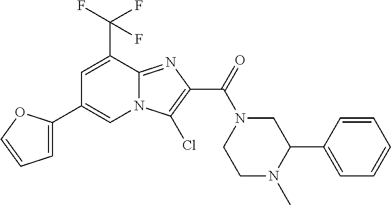 | (3-Chloro-6-furan-2-yl-8- trifluoromethyl-imidazo[1,2-a]pyridin- 2-yl)-(4-methyl-3-phenyl-piperazin-1- yl)-methanone |
| 319 | 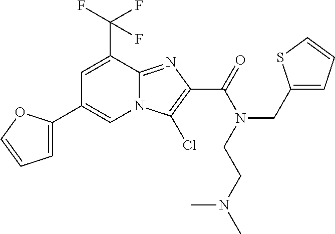 | 3-Chloro-6-furan-2-yl-8- trifluoromethyl-imidazo[1,2-a]pyridine- 2-carboxylic acid (2-dimethylamino- ethyl)-thiophen-2-ylmethyl-amide |
| 320 | 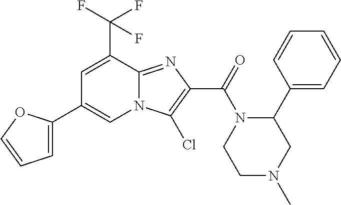 | (3-Chloro-6-furan-2-yl-8- trifluoromethyl-imidazo[1,2-a]pyridin- 2-yl)-(4-methyl-2-phenyl-piperazin-1- yl)-methanone |
| 321 | 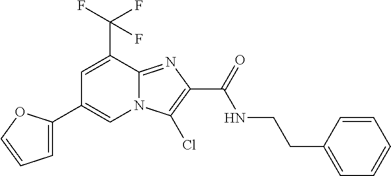 | 3-Chloro-6-furan-2-yl-8- trifluoromethyl-imidazo[1,2-a]pyridine- 2-carboxylic acid phenethyl-amide |
| 322 | 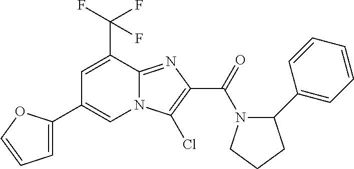 | (3-Chloro-6-furan-2-yl-8- trifluoromethyl-imidazo[1,2-a]pyridin- 2-yl)-(2-phenyl-pyrrolidin-1-yl)- methanone |
| 323 | 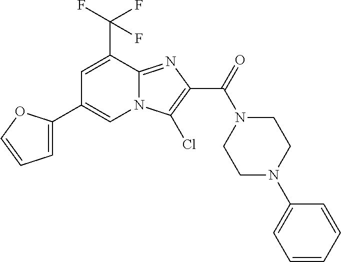 | (3-Chloro-6-furan-2-yl-8- trifluoromethyl-imidazo[1,2-a]pyridin- 2-yl)-(4-phenyl-piperazin-1-yl)- methanone |
| 324 | 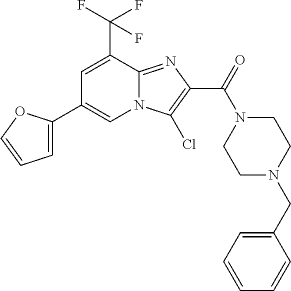 | (4-Benzyl-piperazin-1-yl)-(3-chloro-6- furan-2-yl-8-trifluoromethyl- imidazo[1,2-a]pyridin-2-yl)-methanone |
| 325 | 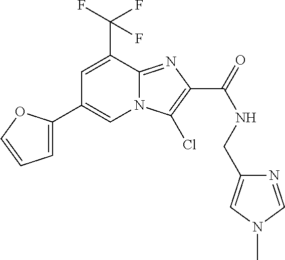 | 3-Chloro-6-furan-2-yl-8- trifluoromethyl-imidazo[1,2-a]pyridine- 2-carboxylic acid (1-methyl-1H- imidazol-4-ylmethyl)-amide |
| 326 | 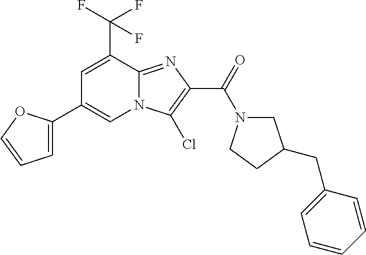 | (3-Benzyl-pyrrolidin-1-yl)-(3-chloro-6- furan-2-yl-8-trifluoromethyl- imidazo[1,2-a]pyridin-2-yl)-methanone |
| 327 | 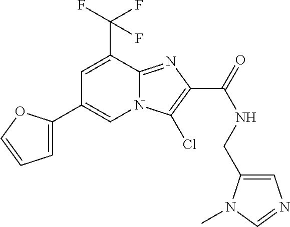 | 3-Chloro-6-furan-2-yl-8- trifluoromethyl-imidazo[1,2-a]pyridine- 2-carboxylic acid (3-methyl-3H- imdiazol-4-ylmethyl)-amide |
| 328 | 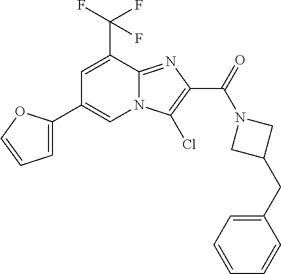 | (3-Benzyl-azetidin-1-yl)-(3-chloro-6- furan-2-yl-8-trifluoromethyl- imidazo[1,2-a]pyridin-2-yl)-methanone |
| 329 | 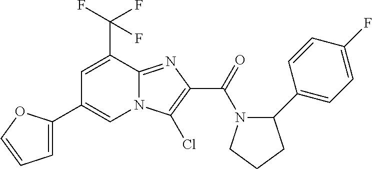 | (3-Chloro-6-furan-2-yl-8- trifluoromethyl-imidazo[1,2-a]pyridin- 2-yl)-[2-(4-fluoro-phenyl)-pyrrolidin-1- yl]-methanone |
| 330 | 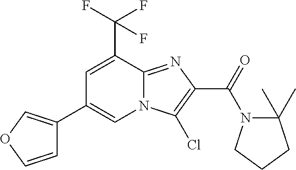 | (3-Chloro-6-furan-3-yl-8- trifluoromethyl-imidazo[1,2-a]pyridin- 2-yl)-(2,2-dimethyl-pyrrolidin-1-yl)- methanone |
| 331 | 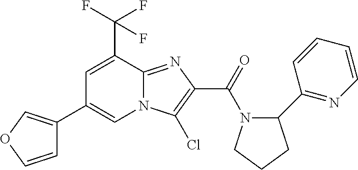 | (3-Chloro-6-furan-3-yl-8- trifluoromethyl-imidazo[1,2-a]pyridin- 2-yl)-(2-pyridin-2-yl-pyrrolidin-1-yl)- methanone |
| 332 | 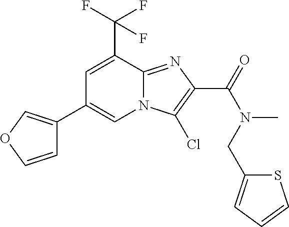 | 3-Chloro-6-furan-3-yl-8- trifluoromethyl-imidazo[1,2-a]pyridine- 2-carboxylic acid methyl-thiophen-2- ylmethyl-amide |
| 333 | 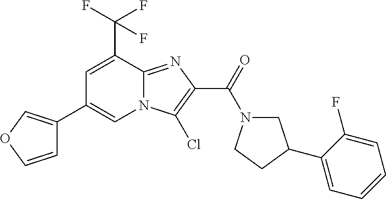 | (3-Chloro-6-furan-3-yl-8- trifluoromethyl-imidazo[1,2-a]pyridin- 2-yl)-[3-(2-fluoro-phenyl)-pyrrolidin-1- yl]-methanone |
| 334 | 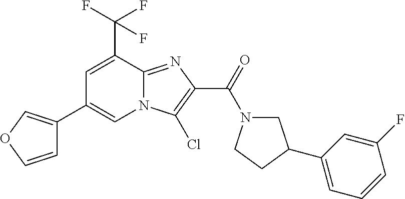 | (3-Chloro-6-furan-3-yl-8- trifluoromethyl-imidazo[1,2-a]pyridin- 2-yl0-[3-(3-fluoro-phenyl)-pyrrolidin-1- yl]-methanone |
| 335 | 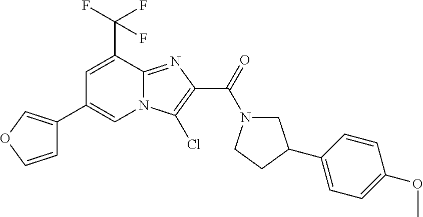 | (3-Chloro-6-furan-3-yl-8- trifluoromethyl-imidazo[1,2-a]pyridin- 2-yl)-[3-(4-methoxy-phenyl)- pyrrolidin-1-yl]-methanone |
| 336 | 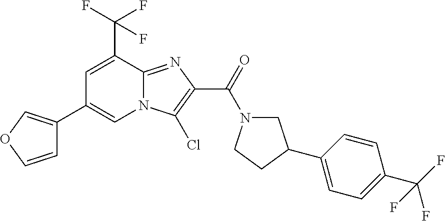 | (3-Chloro-6-furan-3-yl-8- trifluoromethyl-imidazo[1,2-a]pyridin- 2-yl)-[3-(4-trifluoromethyl-phenyl)- pyrrolidin-1-yl]-methanone |
| 337 | 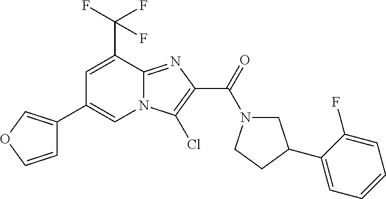 | [3-(2-Fluoro-phenyl)-pyrrolidin-1-yl]- (6-furan-3-yl-8-trifluoromethyl- imidazo[1,2-a]pyridin-2-yl)-methanone |
| 338 | 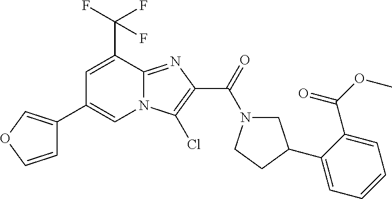 | 2-[1-(3-Chloro-6-furan-3-yl-8- trifluoromethyl-imidazo[1,2-a]pyridine- 2-carbonyl)-pyrrolidin-3-yl]-benzoic acid methyl ester |
| 339 | 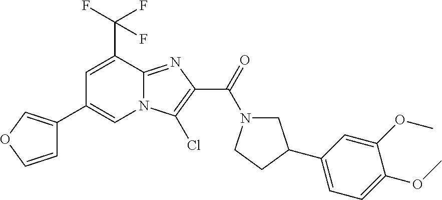 | (3-Chloro-6-furan-3-yl-8- trifluoromethyl-imidazo[1,2-a]pyridin- 2-yl)-[3-(3,4-dimethoxy-phenyl)- pyrrolidin-1-yl]-methanone |
| 340 | 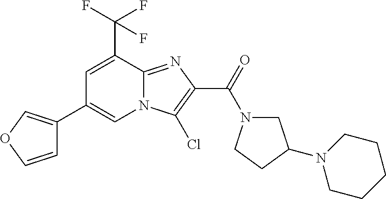 | (3-Chloro-6-furan-3-yl-8- trifluoromethyl-imidazo[1,2-a]pyridin- 2-yl)-(3-piperidin-1-yl-pyrrolidin-1-yl)- methanone |
| 341 | 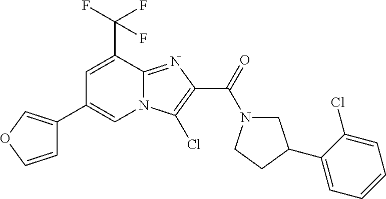 | (3-Chloro-6-furan-3-yl-8- trifluoromethyl-imidazo[1,2-a]pyridin- 2-yl)-[3-(2-chloro-phenyl)-pyrrolidin-1- yl]-methanone |
| 342 | 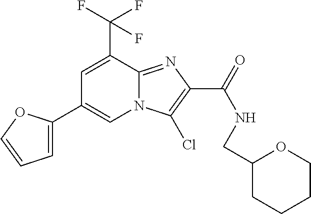 | 3-Chloro-6-furan-2-yl-8- trifluoromethyl-imidazo[1,2-a]pyridine- 2-carboxylic acid (tetrahydro-pyran-2- ylmethyl)-amide |
| 343 | 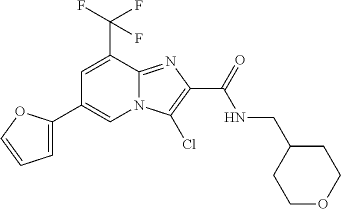 | 3-Chloro-6-furan-2-yl-8- trifluoromethyl-imidazo[1,2-a]pyridine- 2-carboxylic acid (tetrahydro-pyran-4- ylmethyl)-amide |
| 344 | 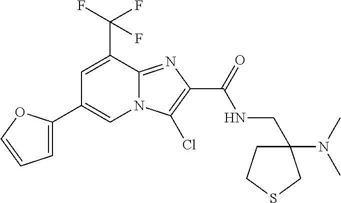 | 3-Chloro-6-furan-2-yl-8- trifluoromethyl-imidazo[1,2-a]pyridine- 2-carboxylic acid (3-dimethylamino- tetrahydro-thiophen-3-ylmethyl)-amide |
| 345 | 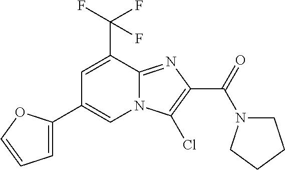 | (3-Chloro-6-furan-2-yl-8- trifluoromethyl-imidazo[1,2-a]pyridin- 2-yl)-pyrrolidin-1-yl-methanone |
| 346 | 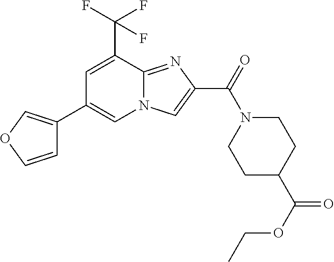 | 1-(6-Furan-3-yl-8-trifluoromethyl- imidazo[1,2-a]pyridine-2-carbonyl)- piperidine-4-carboxylic acid ethyl ester |
| 347 | 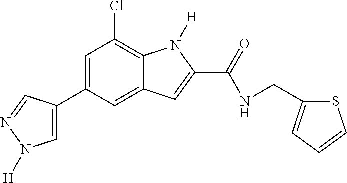 | 7-Chloro-5-(1H-pyrazol-4-yl)-1H- indole-2-carboxylic acid (thiophen-2- ylmethyl)-amide |
| 348 | 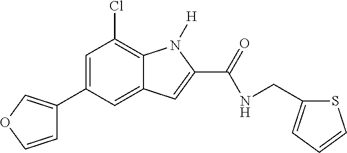 | 7-Chloro-5-furan-3-yl-1H-indole-2- carboxylic acid (thiophen-2-ylmethyl)- amide |
| 349 | 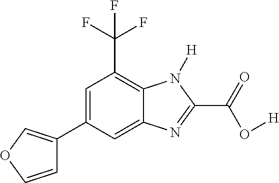 | 5-Furan-3-yl-7-trifluoromethyl-1H- benzoimidazole-2-carboxylic acid |
| 350 | 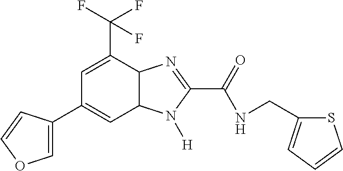 | 6-Furan-3-yl-4-trifluoromethyl-1H- benzoimidazole-2-carboxylic acid (thiophen-2-ylmethyl)-amide or 5-Furan-3-yl-7-trifluoromethyl-1H- benzoimidazole-2-carboxylic acid (thiophen-2-ylmethyl)-amide |
| 351 | 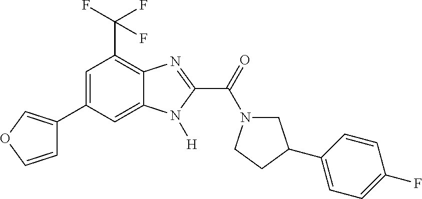 | [3-(4-Fluoro-phenyl)-pyrrolidin-1-yl]- (6-furan-3-yl-4-trilfuoromethyl-1H- benzoimidazol-2-yl)-methanone |
| 352 | 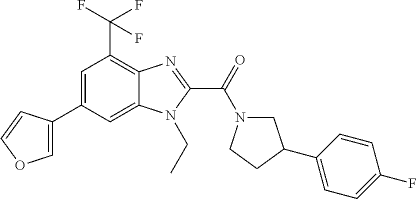 | (1-Ethyl-6-furan-3-yl-4- trilfuoromethyl-1H-benzoimidazol-2- yl)-[3-(4-fluoro-phenyl)-pyrrolidin-1- yl]-methanone |
| 353 | 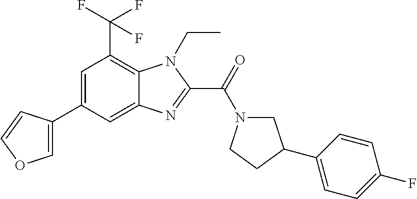 | (1-Ethyl-5-furan-3-yl-7- trifluoromethyl-1H-benzoimidazol-2- yl)-[3-(4-fluoro-phenyl)-pyrrolidin-1- yl]-methanone |
| 354 | 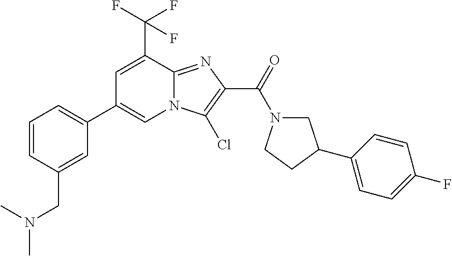 | [3-Chloro-6-(3-dimethylaminomethyl- phenyl)-8-trifluoromethyl-imidazo[1,2- a]pyridin-2-yl]-[3-(4-fluoro-phenyl)- pyrrolidin-1-yl]-methanone |
| 355 | 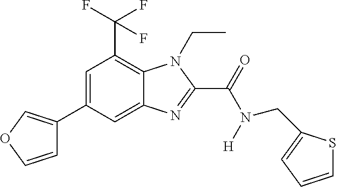 | 1-Ethyl-5-furan-3-yl-7-trifluoromethyl- 1H-benzoimidazole-2-carboxylic acid (thiophen-2-ylmethyl)-amide |
| 356 | 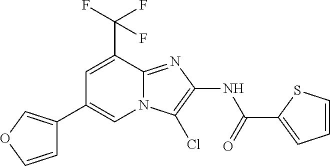 | Thiophene-2-carboxylic acid (3-chloro- 6-furan-3-yl-8-trifluoromethyl- imidazo[1,2-a]pyridin-2-yl)-amide |
| 357 | 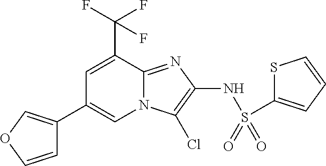 | Thiophene-2-sulfonic acid (3-chloro-6- furan-3-yl-8-trifluoromethyl- imidazo[1,2-a]pyridin-2-yl)-amide |
| 358 | 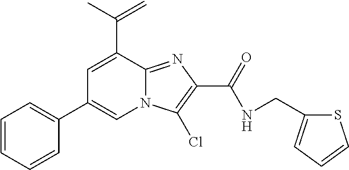 | 3-Chloro-8-isopropenyl-6-phenyl- imidazo[1,2-a]pyridine-2-carboxylic acid (thiophen-2-ylmethyl)-amide |
| 359 | 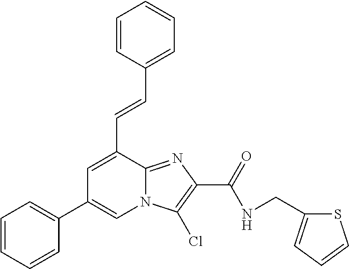 | 3-Chloro-6-phenyl-8-styryl- imidazo[1,2-a]pyridine-2-carboxylic acid (thiophen-2-ylmethyl)-amide |
| 360 | 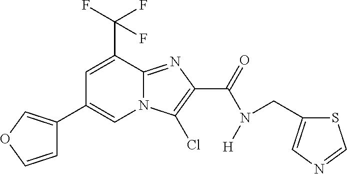 | 3-Chloro-6-furan-3-yl-8- trifluoromethyl-imidazo[1,2-a]pyridine- 2-carboxylic acid (thiazol-5-ylmethyl)- amide |
| 361 | 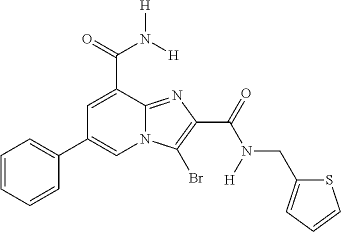 | 3-Bromo-6-phenyl-imidazo[1,2- a]pyridine-2,8-dicarboxylic acid 8- amide 2-[(thiophen-2-ylmethyl)-amide] |
| 362 | 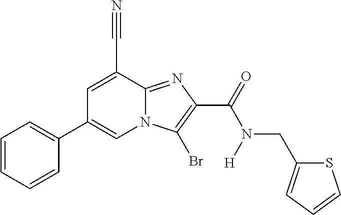 | 3-Bromo-8-cyano-6-phenyl- imidazo[1,2-a]pyridine-2-carboxylic acid (thiophen-2-ylmethyl)-amide |
| 363 | 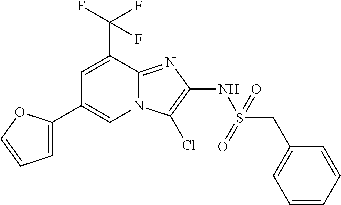 | N-(3-Chloro-6-furan-2-yl-8- trifluoromethyl-imidazo[1,2-a]pyridin- 2-yl-C-phenyl-methanesulfonamide |
| 364 | 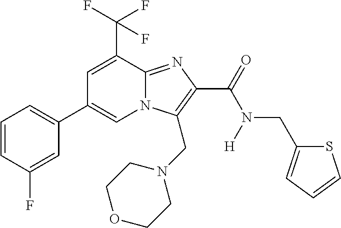 | 6-(3-Fluoro-phenyl)-3-morpholin-4- ylmethyl-8-trifluoromethyl- imidazo[1,2-a]pyridine-2-carboxylic acid (thiophen-2-ylmethyl)-amide |
| 365 | 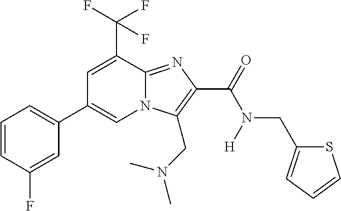 | 3-Dimethylaminomethyl-6-(3-fluoro- phenyl)-8-trifluoromethyl-imidazo[1,2- a]pyridine-2-carboxylic acid (thiophen- 2-ylmethyl)-amide |
| 366 | 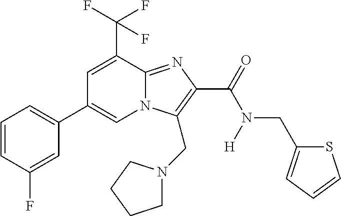 | 6-(3-Fluoro-phenyl)-3-pyrrolidin-1- ylmethyl-8-trifluoromethyl- imidazo[1,2-a]pyridine-2-carboxylic acid (thiophen-2-ylmethyl)-amide |
| 367 | 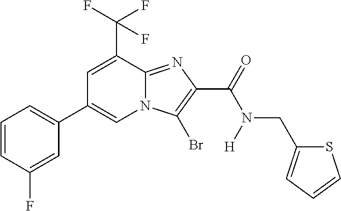 | 3-Bromo-6-(3-fluoro-phenyl)-8- trifluoromethyl-imidazo[1,2-a]pyridine- 2-carboxylic acid (thiophen-2- ylmethyl)-amide |
| 368 | 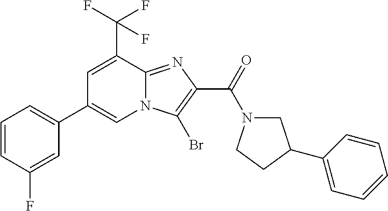 | [3-Bromo-6-(3-fluoro-phenyl)-8- trifluoromethyl-imidazo[1,2-a]pyridin- 2-yl]-(3-phenyl-pyrrolidin-1-yl)- methanone |
| 369 | 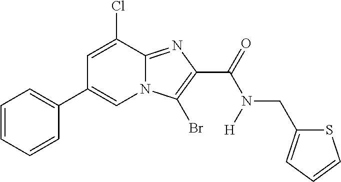 | 3-Bromo-8-chloro-6-phenyl- imidazo[1,2-a]pyridine-2-carboxylic acid (thiophen-2-ylmethyl)-amide |
| 370 | 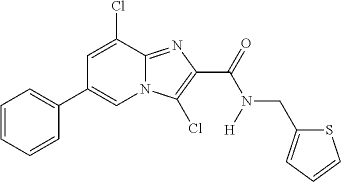 | 3,8-Dichloro-6-phenyl-imidazo[1,2- a]pyridine-2-carboxylic acid (thiophen- 2-ylmethyl)-amide |
| 371 | 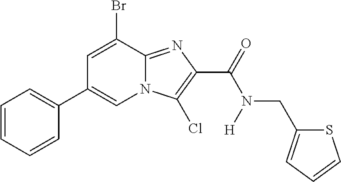 | 8-Bromo-3-chloro-6-phenyl- imidazo[1,2-a]pyridine-2-carboxylic acid (thiophen-2-ylmethyl)-amide |
| 372 | 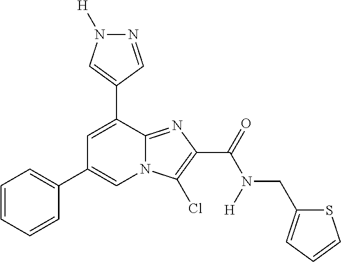 | 3-Chloro-6-phenyl-8-(1H-pyrazol-4- yl)-imidazo[1,2-a]pyridine-2-carboxylic acid (thiophen-2-ylmethyl)-amide |
| 373 | 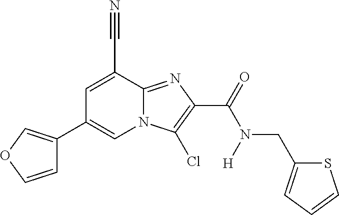 | 3-Chloro-8-cyano-6-furan-3-yl- imidazo[1,2-a]pyridine-2-carboxylic acid (thiophen-2-ylmethyl)-amide |
| 374 | 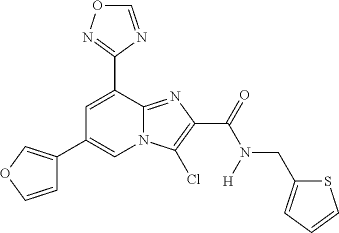 | 3-Chloro-6-furan-3-yl-8- [1,2,4]oxadiazol-3-yl-imidazo[1,2- a]pyridine-2-carboxylic acid (thiophen- 2-ylmethyl)-amide |
| 375 | 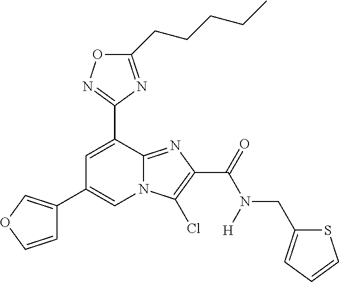 | 3-Chloro-6-furan-3-yl-8-(5-pentyl- [1,2,4]oxadiazol-3-yl)-imidazo[1,2- a]pyridine-2-carboxylic acid (thiophen- 2-ylmethyl)-amide |
| 376 | 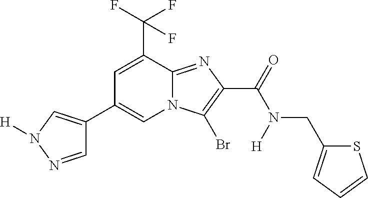 | 3-Bromo-6-(1H-pyrazol-4-yl)-8- trifluoromethyl-imidazo[1,2-a]pyridine- 2-carboxylic acid (thiophen-2- ylmethyl)-amide |
| 377 | 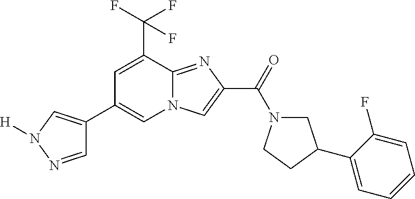 | [3-(2-Fluroo-phenyl)-pyrrolidin-1-yl]- [6-(1H-pyrazol-4-yl)-8-trifluoromethyl- imidazo[1,2-a]pyridin-2-yl]-methanone |
| 378 | 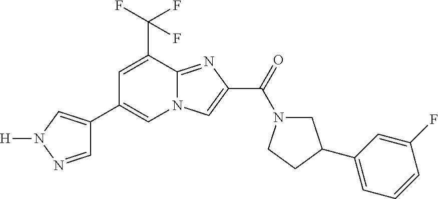 | [3-(3-Fluoro-phenyl)-pyrrolidin-1-yl]- [6-(1H-pyrazol-4-yl)-8-trifluoromethyl- imidazo[1,2-a]pyridin-2-yl]-methanone |
| 379 | 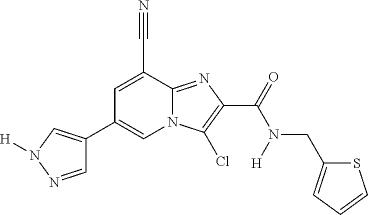 | 3-Chloro-8-cyano-6-(1H-pyrazol-4-yl)- imidazo[1,2-a]pyridine-2-carboxylic acid (thiophen-2-ylmethyl)-amide |
| 380 | 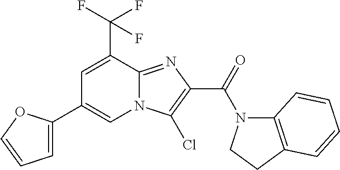 | (3-Chloro-6-furan-2-yl-8- trifluoromethyl-imidazo[1,2-a]pyridin- 2-yl)-(2,3-dihydro-indol-1-yl)- methanone |
| 381 | 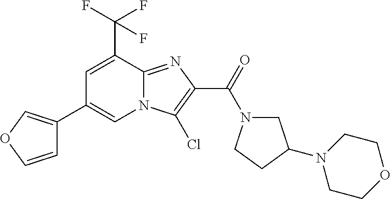 | (3-Chloro-6-furan-3-yl-8- trifluoromethyl-imidazo[1,2-a]pyridin- 2-yl)-(3-morpholin-4-yl-pyrrolidin-1- yl)-methanone |

TABLE 3

| COMP. | | |
|---|---|---|
| # | CHEMISTRY | IUPAC_Name |
| 382 | 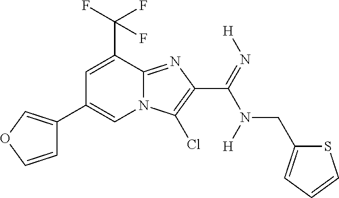 | 3-Chloro-6-furan-3-yl-N- thiophen-2-ylmethyl-8- trifluoromethyl-imidazo[1,2- a]pyridine-2-carboxamidine |
| 383 | 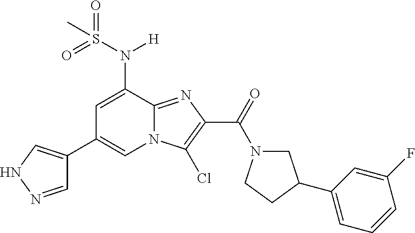 | N-[3-Chloro-2-[3-(3-fluoro- phenyl)-pyrrolidine-1-carbonyl]- 6-(1H-pyrazol-4-yl)-imidazo[1,2- a]pyridin-8-yl]- methanesulfonamide |
| 384 | 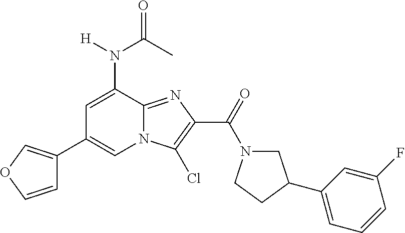 | N-{3-Chloro-2-[3-(3-fluoro- phenyl)-pyrrolidine-1-carbonyl]- 6-furan-3-yl-imidazo[1,2- a]pyridin-8-yl}-acetamide |
| 385 | 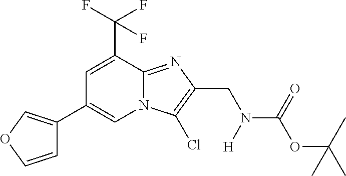 | (3-Chloro-6-furan-3-yl-8- trifluoromethyl-imidazo[1,2- a]pyridin-2-ylmethyl)-carbamic acid tert-butyl ester |
| 386 | 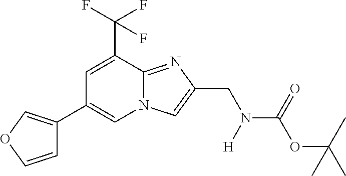 | (6-Furan-3-yl-8-trifluoromethyl- imidazo[1,2-a]pyridin-2- ylmethyl)-carbamic acid tert-butyl ester |
| 387 | 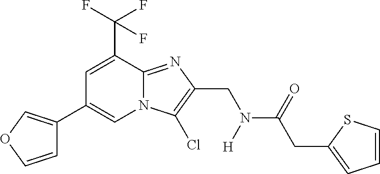 | N-(3-Chloro-6-furan-3-yl-8- trifluoromethyl-imidazo[1,2- a]pyridin-2-ylmethyl)-2-thiophen- 2-yl-acetamide |
| 388 | 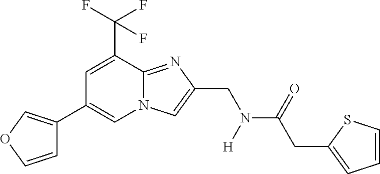 | N-(6-Furan-3-yl-8- trifluoromethyl-imidazo[1,2- a]pyridin-2-ylmethyl)-2-thiophen- 2-yl-acetamide |
| 389 | 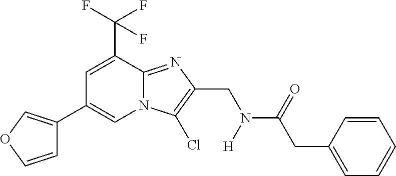 | N-(3-Chloro-6-furan-3-yl-8- trifluoromethyl-imidazo[1,2- a]pyridin-2-ylmethyl)-2-phenyl- acetamide |
| 390 | 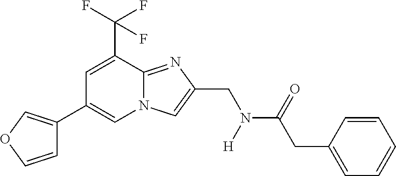 | N-(6-Furan-3-yl-8- trifluoromethyl-imidazo[1,2- a]pyridin-2-ylmethyl)-2-phenyl- acetamide |
| 391 | 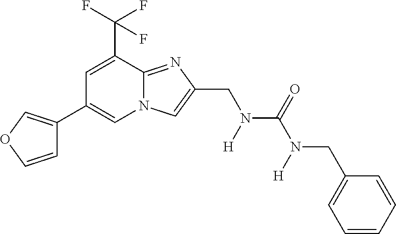 | 1-Benzyl-3-(6-furan-3-yl-8- trifluoromethyl-imidazo[1,2- a]pyridin-2-ylmethyl)-urea |
| 392 | 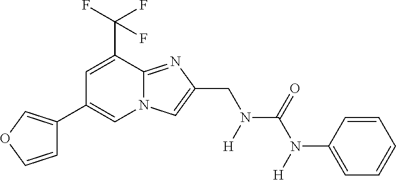 | 1-(6-Furan-3-yl-8- trifluoromethyl-imidazo[1,2- a]pyridin-2-ylmethyl)-3-phenyl- urea |
| 393 | 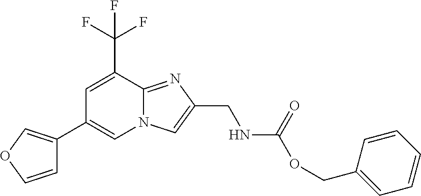 | (6-Furan-3-yl-8-trifluoromethyl- imidazo[1,2-a]pyridin-2- ylmethyl)-carbamic acid benzyl ester |
| 394 | 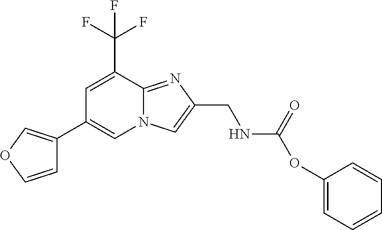 | (6-Furan-3-yl-8-trifluoromethyl- imidazo[1,2-a]pyridin-2- ylmethyl)-carbamic acid phenyl ester |
| 395 | 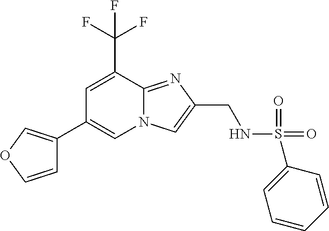 | N-(6-Furan-3-yl-8- trifluoromethyl-imidazo[1,2- a]pyridin-2-ylmethyl)- benzenesulfonamide |
| 396 | 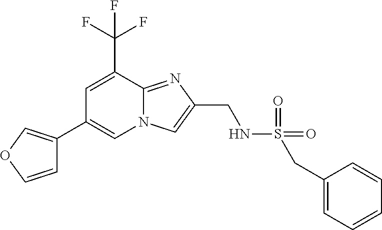 | N-(6-Furan-3-yl-8- trifluoromethyl-imidazo[1,2- a]pyridin-2-ylmethyl)-C-phenyl- methanesulfonamide |
| 397 | 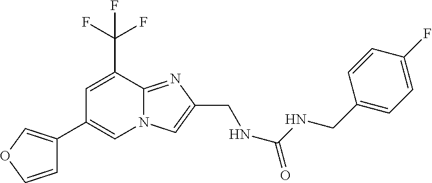 | 1-(4-Fluoro-benzyl)-3-(6-furan-3- yl-8-trifluoromethyl-imidazo[1,2- a]pyridin-2-ylmethyl)-urea |
| 398 | 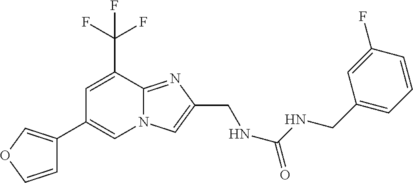 | 1-(3-Fluoro-benzyl)-3-(6-furan-3- yl-8-trifluoromethyl-imidazo[1,2- a]pyridin-2-ylmethyl)-urea |
| 399 | 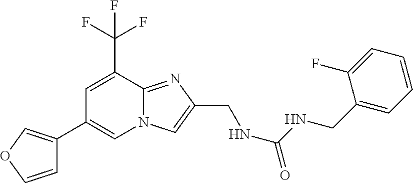 | 1-(2-Fluoro-benzyl)-3-(6-furan-3- yl-8-trifluoromethyl-imidazo[1,2- a]pyridin-2-ylmethyl)-urea |
| 400 | 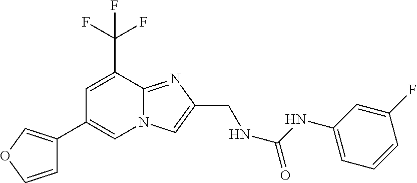 | 1-(3-Fluoro-phenyl)-3-(6-furan-3- yl-8-trifluoromethyl-imidazo[1,2- a]pyridin-2-ylmethyl)-urea |
| 401 | 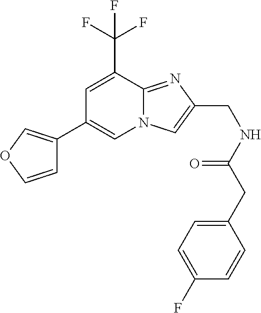 | 2-(4-fluorophenyl)-N-{[6-(furan- 3-yl)-8- (trifluoromethyl)imidazo[1,2- a]pyridin-2-yl]methyl}acetamide |
| 402 | 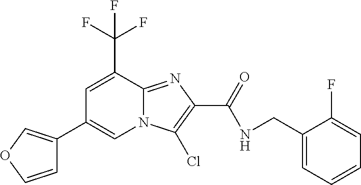 | 3-Chloro-6-furan-3-yl-8- trifluoromethyl-imidazo[1,2- a]pyridine-2-carboxylic acid 2- fluoro-benzylamide |
| 403 | 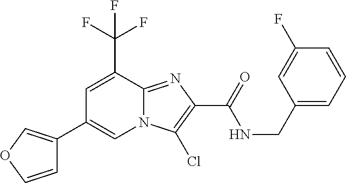 | 3-Chloro-6-furan-3-yl-8- trifluoromethyl-imidazo[1,2- a]pyridine-2-carboxylic acid 3- fluoro-benzylamide |
| 404 | 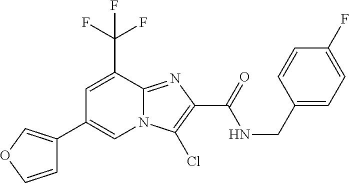 | 3-Chloro-6-furan-3-yl-8- trifluoromethyl-imidazo[1,2- a]pyridine-2-carboxylic acid 4- fluoro-benzylamide |
| 405 | 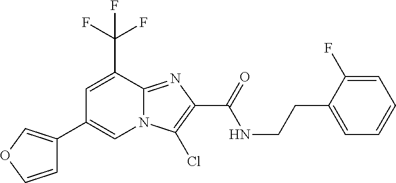 | 3-Chloro-6-furan-3-yl-8- trifluoromethyl-imidazo[1,2- a]pyridine-2-carboxylic acid [2- (2-fluoro-phenyl)-ethyl]-amide |
| 406 | 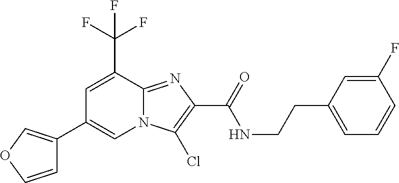 | 3-Chloro-6-furan-3-yl-8- trifluoromethyl-imidazo[1,2- a]pyridine-2-carboxylic acid [2- (3-fluoro-phenyl)-ethyl]-amide |
| 407 | 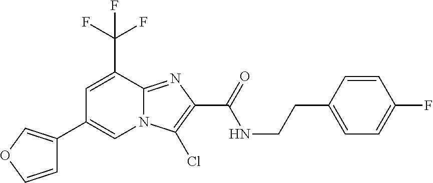 | 3-Chloro-6-furan-3-yl-8- trifluoromethyl-imidazo[1,2- a]pyridine-2-carboxylic acid [2- (4-fluroo-phenyl)-ethyl]-amide |
| 408 | 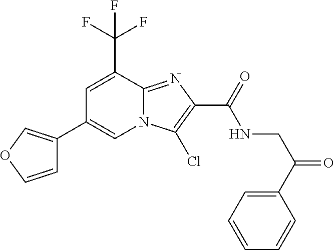 | 3-Chloro-6-furan-3-yl-8- trifluoromethyl-imidazo[1,2- a]pyridine-2-carboxylic acid (2- oxo-2-phenyl-ethyl)-amide |
| 409 | 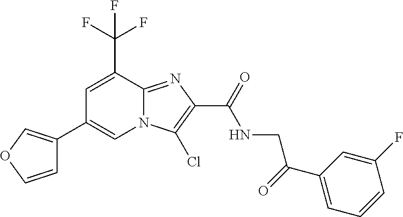 | 3-Chloro-6-furan-3-yl-8- trifluoromethyl-imidazo[1,2- a]pyridine-2-carboxylic acid [2- (3-fluoro-phenyl)-2-oxo-ethyl]- amide |
| 410 | 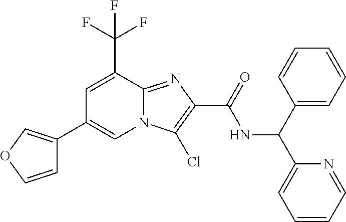 | 3-Chloro-6-furan-3-yl-8- trifluoromethyl-imidazo[1,2- a]pyridine-2-carboxylic acid (phenyl-pyridin-2-yl-methyl)- amide |
| 411 | 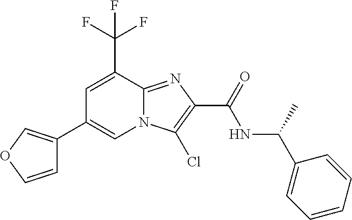 | 3-Chloro-6-furan-3-yl-8- trifluoromethyl-imidazo[1,2- a]pyridine-2-carboxylic acid (1- phenyl-ethyl)-amide |
| 412 | 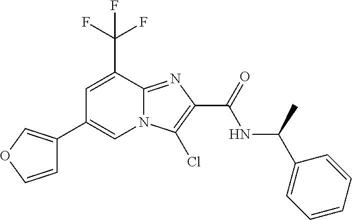 | 3-Chloro-6-furan-3-yl-8- trifluoromethyl-imidazo[1,2- a]pyridine-2-carboxylic acid (1- phenyl-ethyl)-amide |
| 413 | 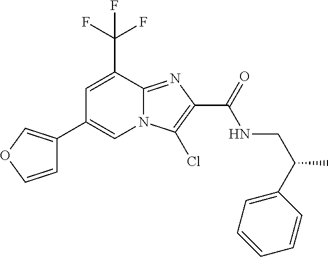 | 3-Chloro-6-furan-3-yl-8- trifluoromethyl-imidazo[1,2- a]pyridine-2-carboxylic acid (2- phenyl-propyl)-amide |
| 414 | 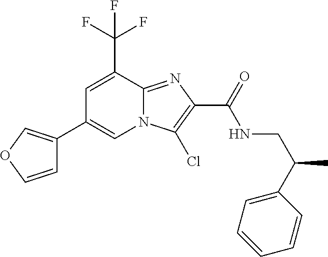 | 3-Chloro-6-furan-3-yl-8- trifluoromethyl-imidazo[1,2- a]pyridine-2-carboxylic acid (2- phenyl-propyl)-amide |
| 415 | 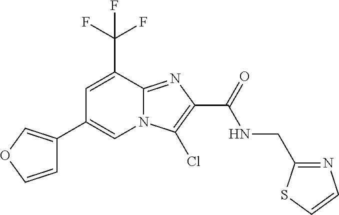 | 3-Chloro-6-furan-3-yl-8- trifluoromethyl-imidazo[1,2- a]pyridine-2-carboxylic acid (thiazol-2-ylmethyl)-amide |
| 416 | 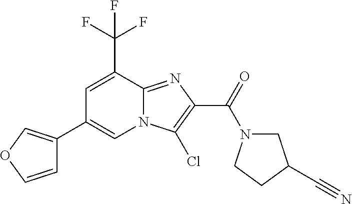 | 1-(3-Chloro-6-furan-3-yl-8- trifluoromethyl-imidazo[1,2- a]pyridine-2-carbonyl)- pyrrolidine-3-carbonitrile |
| 417 | 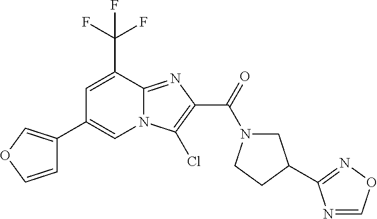 | (3-Chloro-6-furan-3-yl-8- trifluoromethyl-imidazo[1,2- a]pyridin-2-yl)-(3- [1,2,4]oxadiazol-3-yl-pyrrolidin- 1-yl)-methanone |
| 418 | 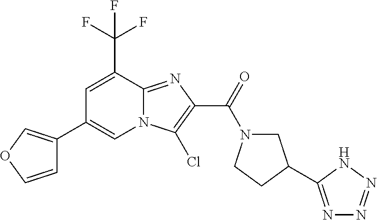 | (3-Chloro-6-furan-3-yl-8- trifluoromethyl-imidazo[1,2- a]pyridin-2-yl)-[3-(1H-tetrazol-5- yl)-pyrrolidin-1-yl]-methanone |
| 419 | 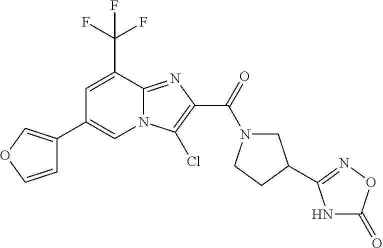 | 3-[1-(3-Chloro-6-furan-3-yl-8- trifluoromethyl-imidazo[1,2- a]pyridine-2-carbonyl)-pyrrolidin- 3-yl]-4H-[1,2,4]oxadiazol-5-one |
| 420 | 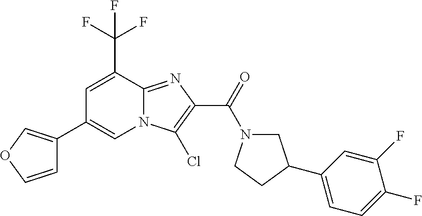 | (3-Chloro-6-furan-3-yl-8- trifluoromethyl-imidazo[1,2- a]pyridin-2-yl)-[3-(3,4-difluoro- phenyl)-pyrrolidin-1-yl]- methanone |
| 421 | 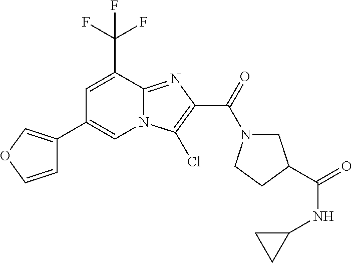 | 1-(3-Chloro-6-furan-3-yl-8- trifluoromethyl-imidazo[1,2- a]pyridine-2-carbonyl)- pyrrolidine-3-carboxylic acid cyclopropylamide |
| 422 | 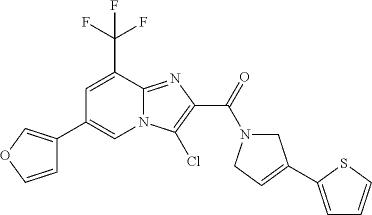 | (3-Chloro-6-furan-3-yl-8- trifluoromethyl-imidazo[1,2- a]pyridin-2-yl)-(3-thiophen-2-yl- 2,5-dihydro-pyrrol-1-yl)- methanone |
| 423 | 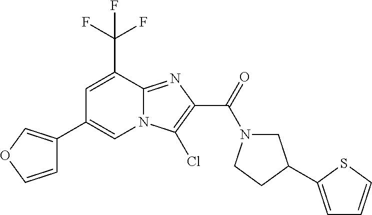 | (3-Chloro-6-furan-3-yl-8- trifluoromethyl-imidazo[1,2- a]pyridin-2-yl)-(3-thiophen-2-yl- pyrrolidin-1-yl)-methanone |
| 424 | 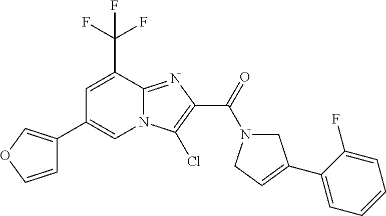 | (3-Chloro-6-furan-3-yl-8- trifluoromethyl-imidazo[1,2- a]pyridin-2-yl)-[3-(2-fluoro- phenyl)-2,5-dihydro-pyrrol-1-yl]- methanone |
| 425 | 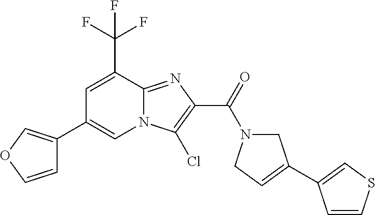 | (3-Chloro-6-furan-3-yl-8- trifluoromethyl-imidazo[1,2- a]pyridin-2-yl)-(3-thiophen-3-yl- 2,5-dihydro-pyrrol-1-yl)- methanone |
| 426 | 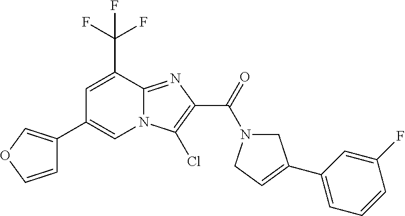 | (3-Chloro-6-furan-3-yl-8- trifluoromethyl-imidazo[1,2- a]pyridin-2-yl)-[3-(3-fluoro- phenyl)-2,5-dihydro-pyrrol-1-yl]- methanone |
| 427 | 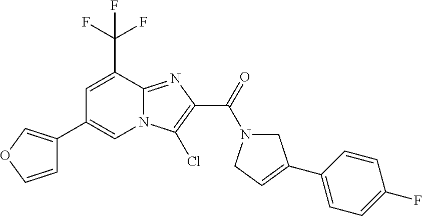 | (3-Chloro-6-furan-3-yl-8- trifluoromethyl-imidazo[1,2- a]pyridin-2-yl)-[3-(4-fluoro- phenyl)-2,5-dihydro-pyrrol-1-yl]- methanone |
| 428 | 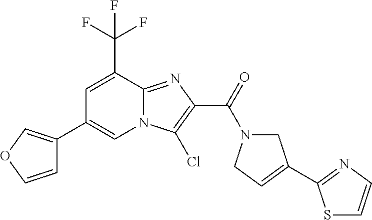 | (3-Chloro-6-furan-3-yl-8- trifluoromethyl-imidazo[1,2- a]pyridin-2-yl)-(3-thiazol-2-yl- 2,5-dihydro-pyrrol-1-yl)- methanone |
| 429 | 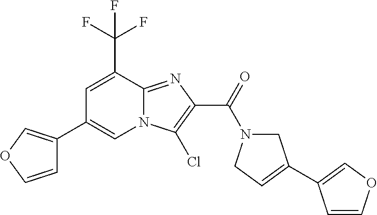 | (3-Chloro-6-furan-3-yl-8- trifluoromethyl-imidazo[1,2- a]pyridin-2-yl)-(3-furan-3-yl-2,5- dihydro-pyrrol-1-yl)-methanone |
| 430 | 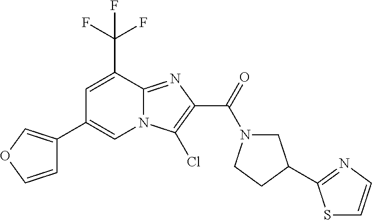 | (3-Chloro-6-furan-3-yl-8- trifluoromethyl-imidazo[1,2- a]pyridin-2-yl)-(3-thiazol-2-yl- pyrrolidin-1-yl)-methanone |
| 431 | 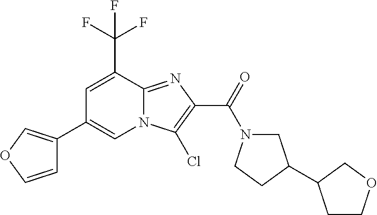 | (3-Chloro-6-furan-3-yl-8- trifluoromethyl-imidazo[1,2- a]pyridin-2-yl)-[3-(tetrahydro- furan-3-yl)-pyrrolidin-1-yl]- methanone |
| 432 | 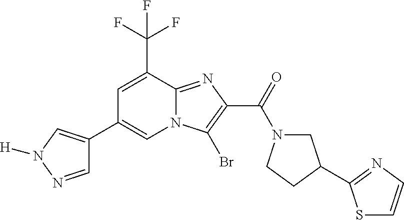 | [3-Bromo-6-(1H-pyrazol-4-yl)-8- trifluoromethyl-imidazo[1,2- a]pyridin-2-yl]-(3-thiazol-2-yl- pyrrolidin-1-yl)-methanone |
| 433 | 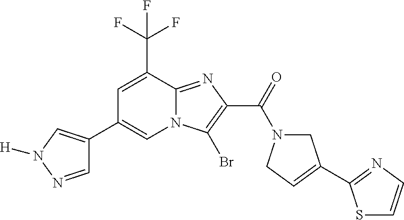 | [3-Bromo-6-(1H-pyrazol-4-yl)-8- trifluoromethyl-imidazo[1,2- a]pyridin-2-yl]-(3-thiazol-2-yl- 2 ,5-dihydro-pyrrol-1-yl)- methanone |
| 434 | 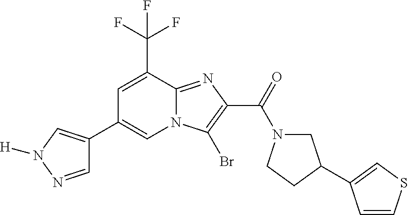 | [3-Bromo-6-(1H-pyrazol-4-yl)-8- trifluoromethyl-imidazo[1,2- a]pyridin-2-yl]-(3-thiophen-3-yl- pyrrolidin-1-yl)-methanone |
| 435 | 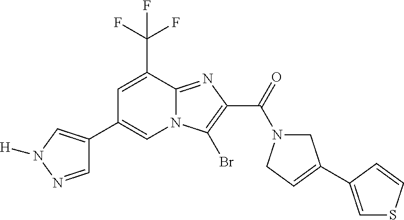 | [3-Bromo-6-(1H-pyrazol-4-yl)-8- trifluoromethyl-imidazo[1,2- a]pyridin-2-yl]-(3-thiophen-3-yl- 2,5-dihydro-pyrrol-1-yl)- methanone |
| 436 | 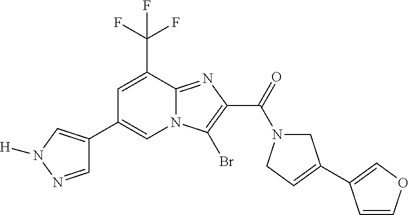 | [3-Bromo-6-(1H-pyrazol-4-yl)-8- trifluoromethyl-imidazo[1,2- a]pyridin-2-yl]-(3-furan-3-yl-2,5- dihydro-pyrrol-1-yl)-methanone |
| 437 | 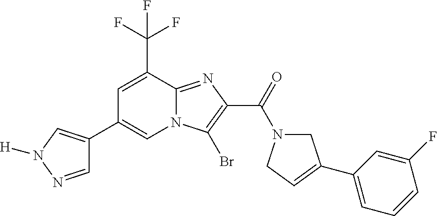 | [3-Bromo-6-(1H-pyrazol-4-yl)-8- trifluoromethyl-imidazo[1,2- a]pyridin-2-yl]-[3-(3-fluroo- phenyl)-2,5-dihydro-pyrrol-1-yl]- methanone |
| 438 | 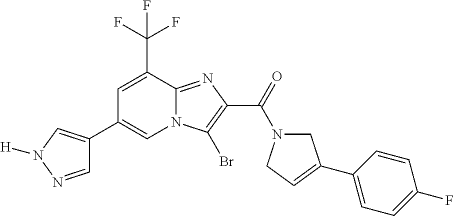 | [3-Bromo-6-(1H-pyrazol-4-yl)-8- trifluoromethyl-imidazo[1,2- a]pyridin-2-yl]-[3-(4-fluoro- phenyl)-2,5-dihydro-pyrrol-1-yl]- methanone |
| 439 | 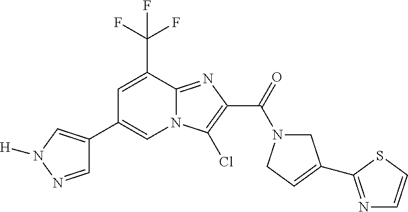 | [3-Chloro-6-(1H-pyrazol-4-yl)-8- (trifluoromethyl)imidazo[1,2- a]pyridin-2-yl][3-(1,3-thiazol-2- yl)-2,5-dihydro-1H-pyrrol-1- yl]methanone |
| 440 | 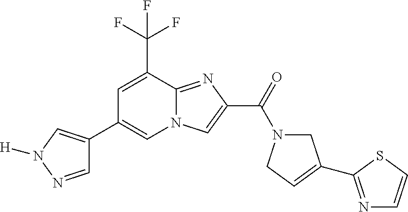 | [6-(1H-pyrazol-4-yl)-8- (trifluoromethyl)imidazo[1,2- a]pyridin-2-yl][3-(1,3-thiazol-2- yl)-2,5-dihydro-1H-pyrrol-1- yl]methanone |
| 441 | 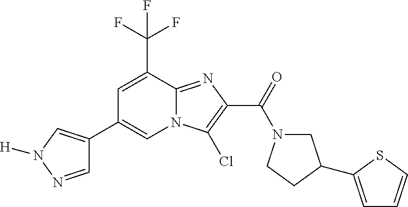 | [3-chloro-6-(1H-pyrazol-4-yl)-8- (trifluoromethyl)imidazo[1,2- a]pyridin-2-yl][3-(thiophen-2- yl)pyrrolidin-1-yl]methanone |
| 442 | 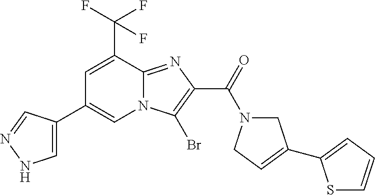 | [3-Bromo-6-(1H-pyrazol-4-yl)-8- trifluoromethyl-imidazo[1,2- a]pyridin-2-yl]-(3-thiophen-2-yl- 2,5-dihydro-pyrrol-1-yl)- methanone |
| 443 | 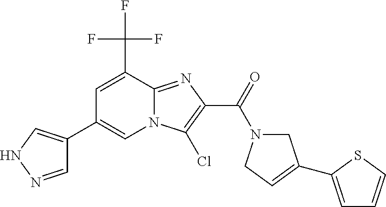 | [3-chloro-6-(1H-pyrazol-4-yl)-8- (trifluoromethyl)imidazo[1,2- a]pyridin-2-yl][3-(thiophen-2-yl)- 2,5-dihydro-1H-pyrrol-1- yl]methanone |
| 444 | 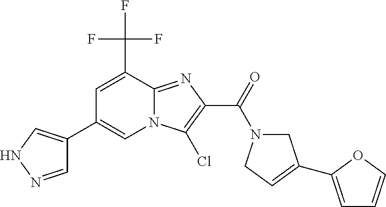 | [3-chloro-6-(1H-pyrazol-4-yl)-8- (trifluoromethyl)imidazo[1,2- a]pyridin-2-yl][3-(furan-2-yl)-2,5- dihydro-1H-pyrrol-1- yl]methanone |
| 445 | 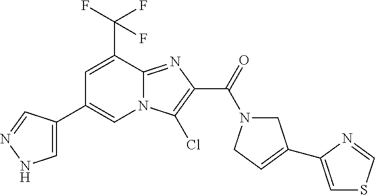 | [3-chloro-6-(1H-pyrazol-4-yl)-8- (trifluoromethyl)imidazo[1,2- a]pyridin-2-yl][3-(1,3-thiazol-4- yl)-2,5-dihydro-1H-pyrrol-1- yl]methanone |
| 446 | 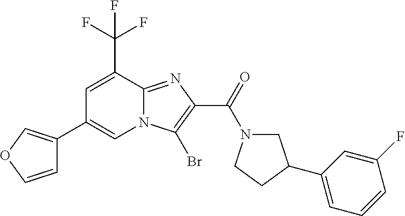 | (3-Bromo-6-furan-3-yl-8- trifluoromethyl-imidazo[1,2- a]pyridin-2-yl)-[3-(3-fluoro- phenyl)-pyrrolidin-1-yl]- methanone |
| 447 | 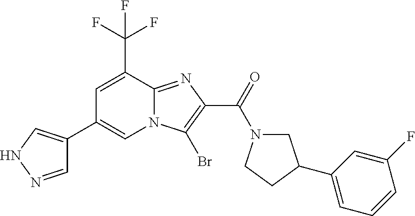 | [3-Bromo-6-(1H-pyrazol-4-yl)-8- trifluoromethyl-imidazo[1,2- a]pyridin-2-yl]-[3-(3-fluoro- phenyl)-pyrrolidin-1-yl]- methanone |
| 448 | 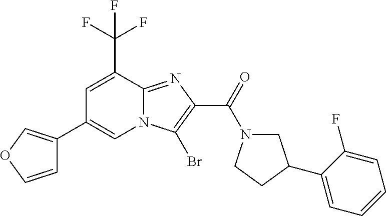 | (3-Bromo-6-furan-3-yl-8- trifluoromethyl-imidazo[1,2- a]pyridin2--yl)-[3-(2-fluoro- phenyl)-pyrrolidin-1-yl]- methanone |
| 449 | 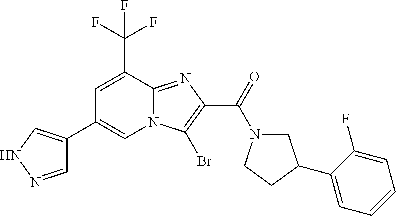 | [3-Bromo-6-(1H-pyrazol-4-yl)-8- trifluoromethyl-imidazo[1,2- a]pyridin-2-yl]-[3-(2-fluoro- phenyl)-pyrrolidin-1-yl]- methanone |
| 450 | 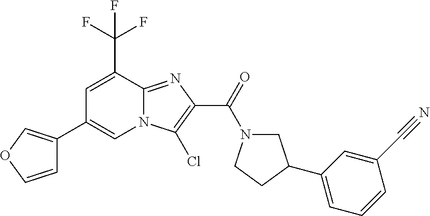 | 3-[1-(3-Chloro-6-furan-3-yl-8- trifluoromethyl-imidazo[1,2- a]pyridine-2-carbonyl)-pyrrolidin- 3-yl]-benzonitrile |
| 451 | 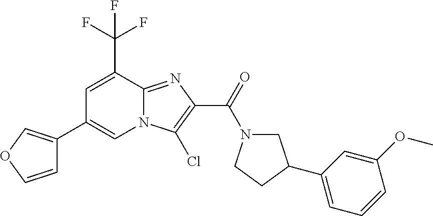 | (3-Chloro-6-furan-3-yl-8- trifluoromethyl-imidazo[1,2- a]pyridin-2-yl)-[3-(3-methoxy- phenyl)-pyrrolidin-1-yl]- methanone |
| 452 | 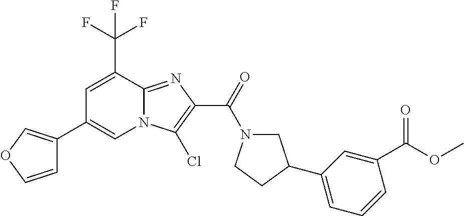 | 3-[1-(3-Chloro-6-furan-3-yl-8- trifluoromethyl-imidazo[1,2- a]pyridine-2-carbonyl)-pyrrolidin- 3-yl]-benzoic acid methyl ester |
| 453 | 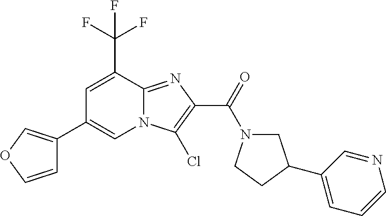 | (3-Chloro-6-furan-3-yl-8- trifluoromethyl-imidazo[1,2- a]pyridin-2-yl)-(3-pyridin-3-yl- pyrrolidin-1-yl)-methanone |
| 454 | 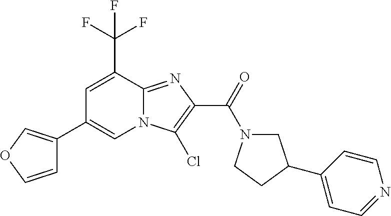 | (3-Chloro-6-furan-3-yl-8- trifluoromethyl-imidazo[1,2- a]pyridin-2-yl)-(3-pyridin-4-yl- pyrrolidin-1-yl)-methanone |
| 455 | 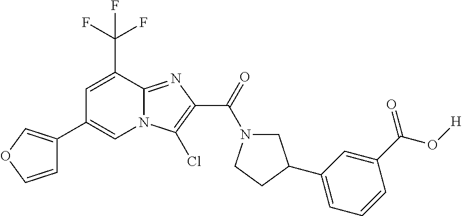 | 3-[1-(3-Chloro-6-furan-3-yl-8- trifluoromethyl-imidazo[1,2- a]pyridine-2-carbonyl)-pyrrolidin- 3-yl]-benzoic acid |
| 456 | 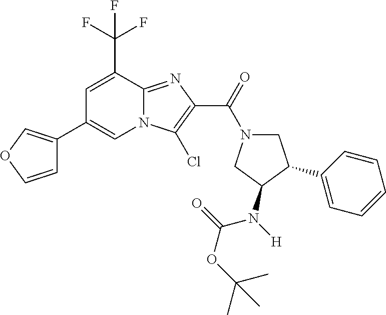 | [1-(3-Chloro-6-furan-3-yl-8- trifluoromethyl-imidazo[1,2- a]pyridine-2-carbonyl)-4-phenyl- pyrrolidin-3-yl]-carbamic acid tert-butyl ester |
| 457 | 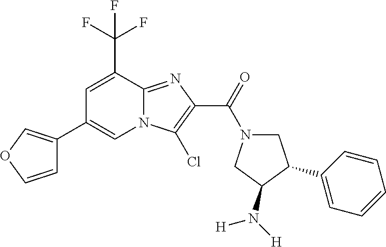 | (3-Amino-4-phenyl-pyrrolidin-1- yl)-(3-chloro-6-furan-3-yl-8- trifluoromethyl-imidazo[1,2- a]pyridin-2-yl)-methanone |
| 458 | 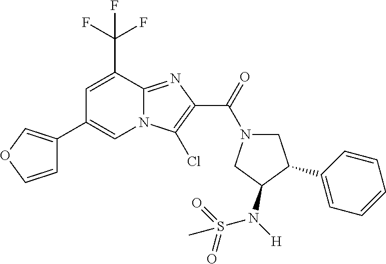 | N-[1-(3-Chloro-6-furan-3-yl-8- trifluoromethyl-imidazo[1,2- a]pyridine-2-carbonyl)-4-phenyl- pyrrolidin-3-yl]- methanesulfonamide |
| 459 | 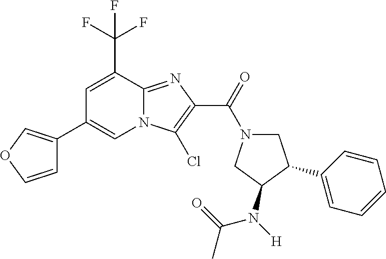 | N-[1-(3-Chloro-6-furan-3-yl-8- trifluoromethyl-imidazo[1,2- a]pyridine-2-carbonyl)-4-phenyl- pyrrolidine-3-yl]-acetamide |
| 460 | 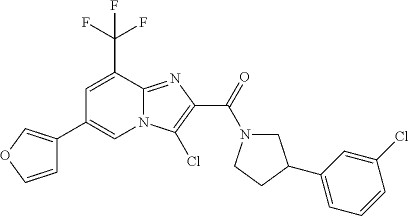 | (3-Chloro-6-furan-3-yl-8- trifluoromethyl-imidazo[1,2- a]pyridin-2-yl)-[3-(3-chloro- phenyl)-pyrrolidin-1-yl]- methanone |
| 461 | 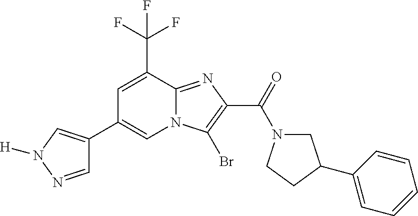 | [3-Bromo-6-(1H-pyrazol-4-yl)-8- trifluoromethyl-imidazo[1,2- a]pyridin-2-yl]-(3-phenyl- pyrrolidin-1-yl)-methanone |
| 462 | 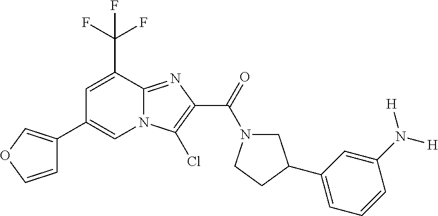 | [3-(3-Amino-phenyl)-pyrrolidin- 1-yl]-(3-chloro-6-furan-3-yl-8- trifluoromethyl-imidazo[1,2- a]pyridin-2-yl)-methanone |
| 463 | 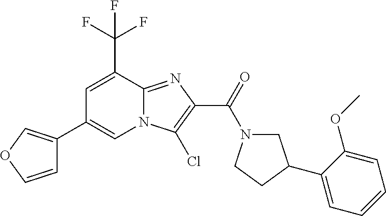 | (3-Chloro-6-furan-3-yl-8- trifluoromethyl-imidazo[1,2- a]pyridin-2-yl)-[3-(2-methoxy- phenyl)-pyrrolidin-1-yl]- methanone |
| 464 | 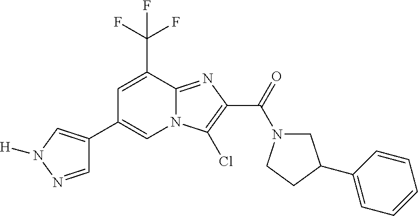 | [3-Chloro-6-(1H-pyrazol-4-yl)-8- trifluoromethyl-imidazo[1,2- a]pyridin-2-yl]-(3-phenyl- pyrrolidin-1-yl)-methanone |
| 465 | 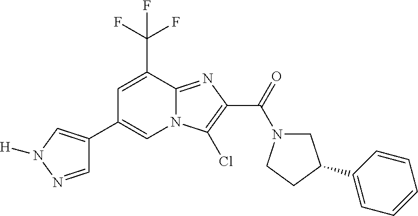 | [3-Chloro-6-(1H-pyrazol-4-yl)-8- trifluoromethyl-imidazo[1,2- a]pyridin-2-yl]-(3-(S)-phenyl- pyrrolidin-1-yl)-methanone |
| 466 | 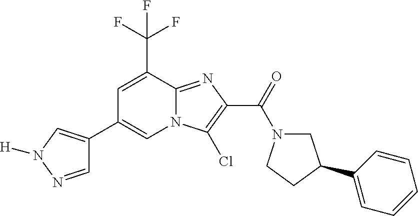 | [3-Chloro-6-(1H-pyrazol-4-yl)-8- trifluoromethyl-imidazo[1,2- a]pyridin-2-yl]-(3-(R)-phenyl- pyrrolidin-1-yl)-methanone |
| 468 | 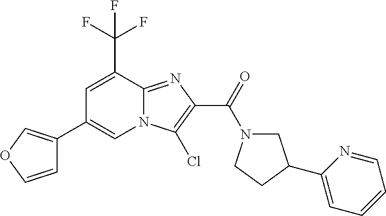 | (3-Chloro-6-furan-3-yl-8- trifluoromethyl-imidazo[1,2- a]pyridin-2-yl)-(3-pyridin-2-yl- pyrrolidin-1-yl)-methanone |
| 472 | 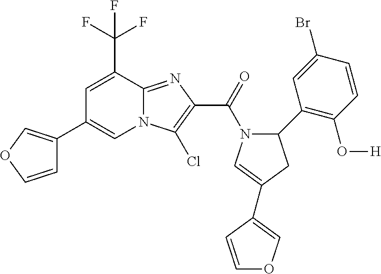 | [5-(5-Bromo-2-hydroxy-phenyl)- 3-furan-3-yl-4,5-dihydro-pyrazol- 1-yl]-(3-chloro-6-furan-3-yl-8- trifluoromethyl-imidazo[1,2- a]pyridin-2-yl)-methanone |
| 474 | 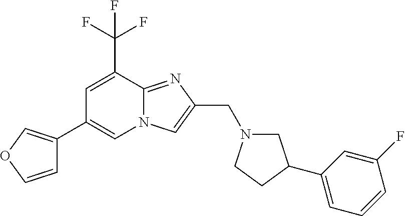 | 2-[3-(3-Fluoro-phenyl)- pyrrolidin-1-ylmethyl]-6-furan-3- yl-8-trifluoromethyl-imidazo[1,2- a]pyridine |
| 475 | 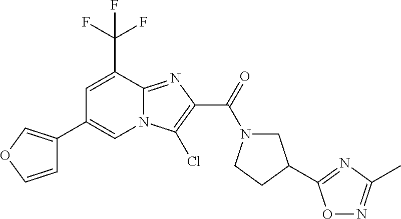 | (3-Chloro-6-furan-3-yl-8- trifluoromethyl-imidazo[1,2- a]pyridin-2-yl)-[3-(3-methyl- [1,2,4]oxadiazol-5-yl)-pyrrolidin- 1-yl]-methanone |
| 476 | 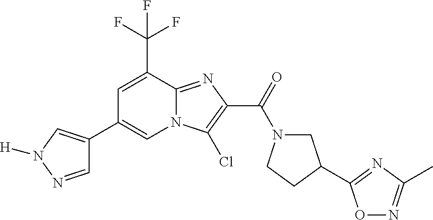 | [3-Chloro-6-(1H-pyrazol-4-yl)-8- trifluoromethyl-imidazo[1,2- a]pyridin-2-yl]-[3-(3-methyl- [1,2,4]oxadiazol-5-yl)-pyrrolidin- 1-yl]-methanone |
| 477 | 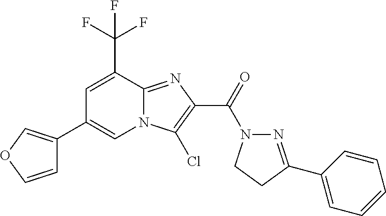 | (3-Chloro-6-furan-3-yl-8- trifluoromethyl-imidazo[1,2- a]pyridin-2-yl)-(3-phenyl-4,5- dihydro-pyrazol-1-yl)-methanone |
| 478 | 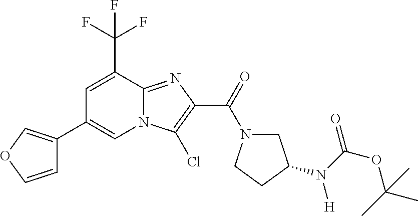 | [1-(3-Chloro-6-furan-3-yl-8- trifluoromethyl-imidazo[1,2- a]pyridine-2-carbonyl)-pyrrolidin- 3-yl]-carbamic acid tert-butyl ester |
| 479 | 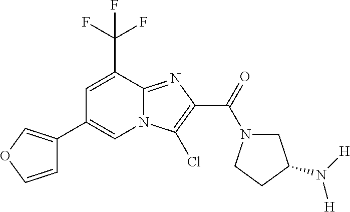 | (3-Amino-pyrrolidin-1-yl)-(3- chloro-6-furan-3-yl-8- trifluoromethyl-imidazo[1,2- a]pyridin-2-yl)-methanone |
| 480 | 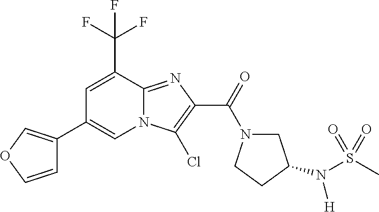 | N-[1-(3-Chloro-6-furan-3-yl-8- trifluoromethyl-imidazo[1,2- a]pyridine-2-carbonyl)-pyrrolidin- 3-yl]-methanesulfonamide |
| 481 | 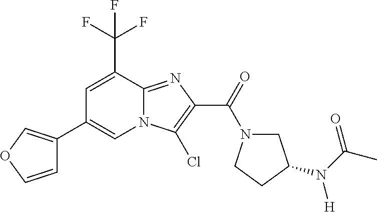 | N-[1-(3-Chloro-6-furan-3-yl-8- trifluoromethyl-imidazo[1,2- a]pyridine-2-carbonyl)-pyrrolidin- 3-yl]-acetamide |
| 482 | 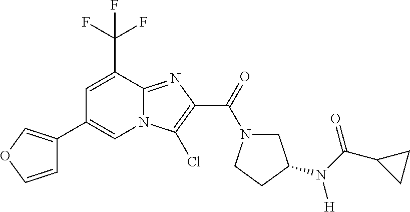 | Cyclopropanecarboxylic acid [1- (3-chloro-6-furan-3-yl-8- trifluoromethyl-imidazo[1,2- a]pyridine-2-carbonyl)-pyrrolidin- 3-yl]-amide |
| 483 | 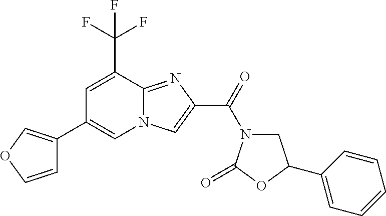 | 3-(6-Furan-3-yl-8- trifluoromethyl-imidazo[1,2- a]pyridin-2-ylmethyl)-5-phenyl- oxazolidin-2-one |
| 484 | 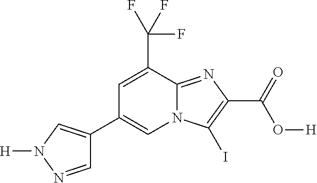 | 3-Iodo-6-(1H-pyrazol-4-yl)-8- trifluoromethyl-imidazo[1,2- a]pyridine-2-carboxylic acid |
| 485 | 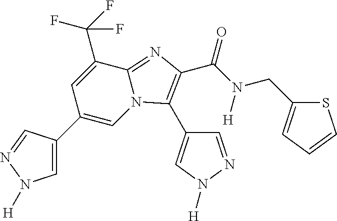 | 3,6-Bis-(1H-pyrazol-4-yl)-8- trifluoromethyl-imidazo[1,2- a]pyridine-2-carboxylic acid (thiophen-2-ylmethyl)-amide |
| 486 | 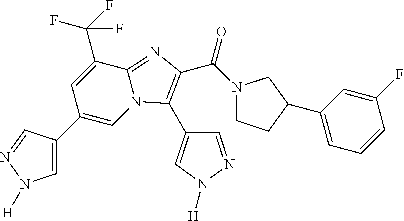 | [3,6-Bis-(1H-pyrazol-4-yl)-8- trifluoromethyl-imidazo[1,2- a]pyridin-2-yl]-[3-(3-fluoro- phenyl)-pyrrolidin-1-yl]- methanone |
| 487 | 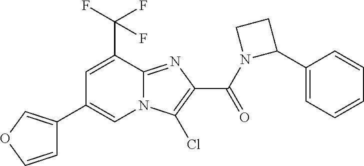 | (3-Chloro-6-furan-3-yl-8- trifluoromethyl-imidazo[1,2- a]pyridin-2-yl)-(2-phenyl- azetidin-1-yl)-methanone |
| 488 | 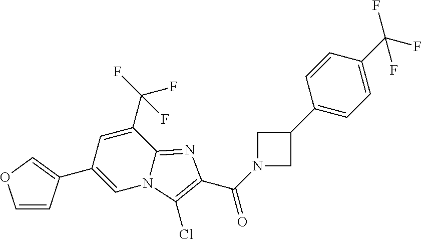 | [3-chloro-6-(furan-3-yl)-8- (trifluoromethyl)imidazo[1,2- a]pyridin-2-yl]{3-[4- (trifluoromethyl)phenyl]azetidin- 1-yl}methanone |
| 489 | 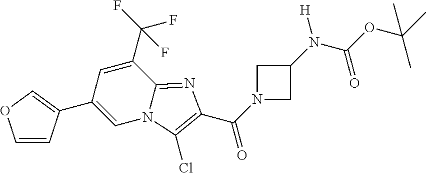 | [1-(3-Chloro-6-furan-3-yl-8- trifluoromethyl-imidazo[1,2- a]pyridine-2-carbonyl)-azetidin-3- yl]-carbamic acid tert-butyl ester |
| 490 | 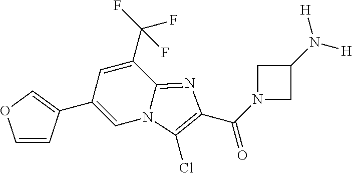 | (3-Amino-azetidin-1-yl)-(3- chloro-6-furan-3-yl-8- trifluoromethyl-imidazo[1,2- a]pyridin-2-yl)-methanone |
| 491 | 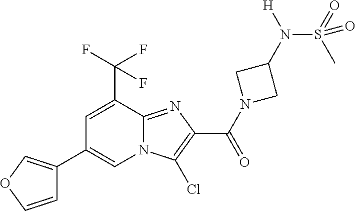 | N-[1-(3-Chloro-6-furan-3-yl-8- trifluoromethyl-imidazo[1,2- a]pyridine-2-carbonyl)-azetidin-3- yl]-methanesulfonamide |
| 492 | 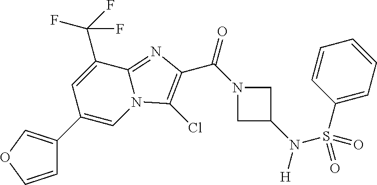 | N-[1-(3-Chloro-6-furan-3-yl-8- trifluoromethyl-imidazo[1,2- a]pyridine-2-carbonyl)-azetidin-3- yl]-benzenesulfonamide |
| 493 | 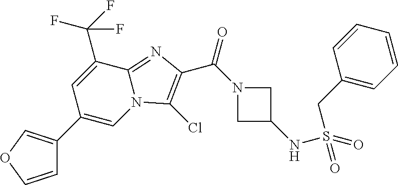 | N-[1-(3-Chloro-6-furan-3-yl-8- trifluoromethyl-imidazo[1,2- a]pyridine-2-carbonyl)-azetidin-3- yl]-C-phenyl-methanesulfonamide |
| 494 | 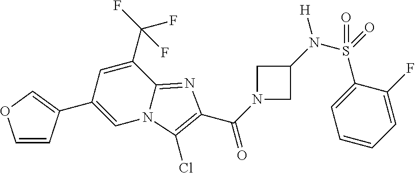 | N-(1-{[3-chloro-6-(furan-3-yl)-8- (trifluoromethyl)imidazo[1,2- a]pyridin-2-yl]carbonyl}azetidin- 3-yl)-2-fluorobenzenesulfonamide |
| 495 | 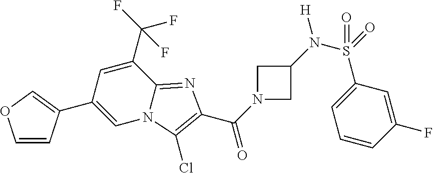 | N-(1-{[3-chloro-6-(furan-3-yl)-8- (trifluoromethyl)imidazo[1,2- a]pyridin-2-yl]carbonyl}azetidin- 3-yl)-3-fluorobenzenesulfonamide |
| 496 | 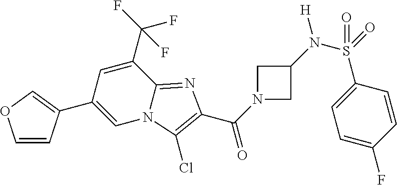 | N-(1-{[3-chloro-6-(furan-3-yl)-8- (trifluoromethyl)imidazo[1,2- a]pyridin-2-yl]carbonyl}azetidin- 3-yl)-4-fluorobenzenesulfonamide |
| 497 | 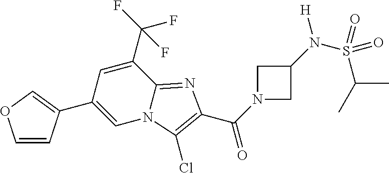 | N-(1-{[3-chloro-6-(furan-3-yl)-8- (trifluoromethyl)imidazo[1,2- a]pyridin-2-yl]carbonyl}azetidin- 3-yl)propane-2-sulfonamide |
| 498 | 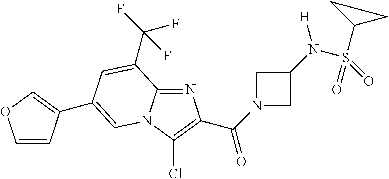 | N-(1-{[3-chloro-6-(furan-3-yl)-8- (trifluoromethyl)imidazo[1,2- a]pyridin-2-yl]carbonyl}azetidin- 3-yl)cyclopropanesulfonamide |
| 499 | 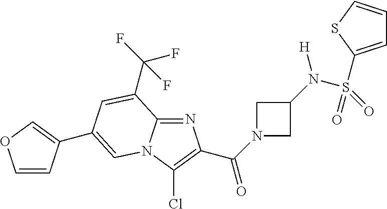 | N-(1-{[3-chloro-6-(furan-3-yl)-8- (trifluoromethyl)imidazo[1,2- a]pyridin-2-yl]carbonyl}azetidin- 3-yl)thiophene-2-sulfonamide |
| 500 | 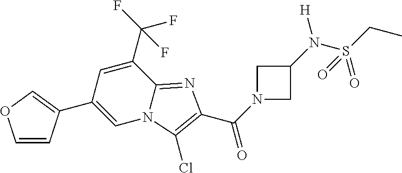 | N-(1-{[3-chloro-6-(furan-3-yl)-8- (trifluoromethyl)imidazo[1,2- a]pyridin-2-yl]carbonyl}azetidin- 3-yl)ethanesulfonamide |
| 501 | 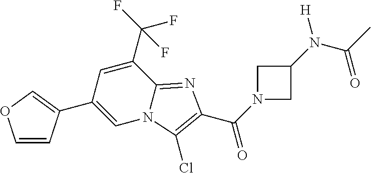 | N-[1-(3-Chloro-6-furan-3-yl-8- trifluoromethyl-imidazo[1,2- a]pyridine-2-carbonyl)-azetidin-3- yl]-acetamide |
| 502 | 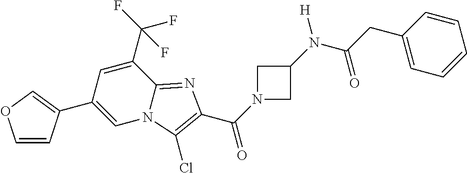 | N-[1-(3-Chloro-6-furan-3-yl-8- trifluoromethyl-imidazo[1,2- a]pyridine-2-carbonyl)-azetidin-3- yl]-2-phenyl-acetamide |
| 503 | 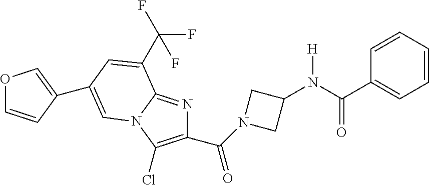 | N-[1-(3-Chloro-6-furan-3-yl-8- trifluoromethyl-imidazo[1,2- a]pyridine-2-carbonyl)-azetidin-3- yl]-benzamide |
| 504 | 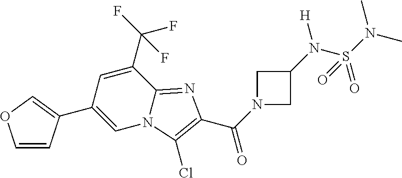 | N′-(1-{[3-chloro-6-(furan-3-yl)-8- (trifluoromethyl)imidazo[1,2- a]pyridin-2-yl]carbonyl}azetidin- 3-yl)-N,N-dimethylsulfuric diamide |
| 505 | 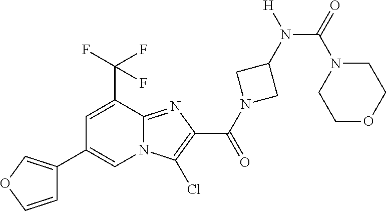 | Morpholine-4-carboxylic acid [1- (3-chloro-6-furan-3-yl-8- trifluoromethyl-imidazo[1,2- a]pyridine-2-carbonyl)-azetidin-3- yl]-amide |
| 506 | 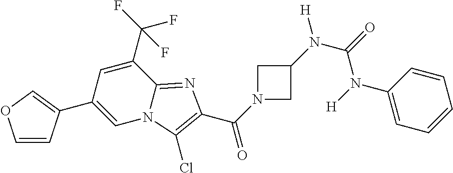 | 1-[1-(3-Chloro-6-furan-3-yl-8- trifluoromethyl-imidazo[1,2- a]pyridine-2-carbonyl)-azetidin-3- yl]-3-phenyl-urea |
| 507 | 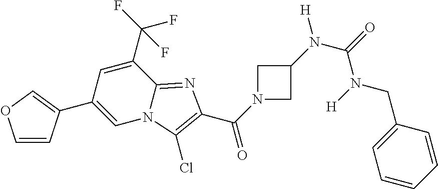 | 1-Benzyl-3-[1-(3-chloro-6-furan- 3-yl-8-trifluoromethyl- imidazo[1,2-a]pyridine-2- carbonyl)-azetidin-3-yl]-urea |
| 508 | 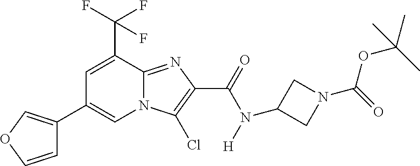 | 3-[(3-Chloro-6-furan-3-yl-8- trifluoromethyl-imidazo[1,2- a]pyridine-2-carbonyl)-amino]- azetidine-1-carboxylic acid tert- butyl ester |
| 509 | 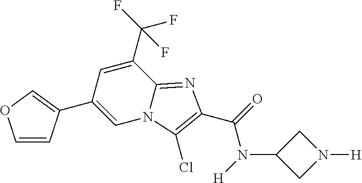 | 3-Chloro-6-furan-3-yl-8- trifluoromethyl-imidazo[1,2- a]pyridine-2-carboxylic acid azetidin-3-ylamide |
| 510 | 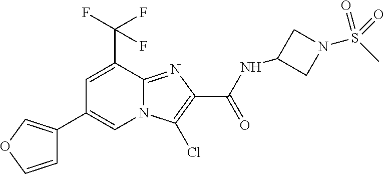 | 3-Chloro-6-furan-3-yl-8- trifluoromethyl-imidazo[1,2- a]pyridine-2-carboxylic acid (1- methanesulfonyl-azetidin-3-yl)- amide |
| 511 | 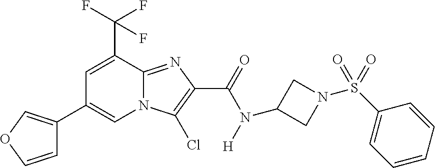 | 3-Chloro-6-furan-3-yl-8- trifluoromethyl-imidazo[1,2- a]pyridine-2-carboxylic acid (1- benzenesulfonyl-azetidin-3-yl)- amide |
| 512 | 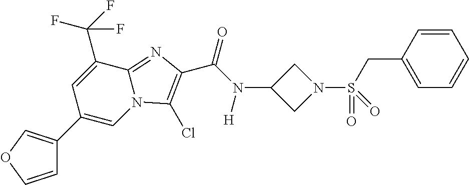 | 3-Chloro-6-furan-3-yl-8- trifluoromethyl-imidazo[1,2- a]pyridine-2-carboxylic acid (1- phenylmethanesulfonyl-azetiidn- 3-yl)-amide |
| 513 | 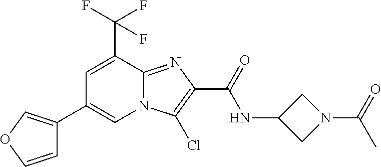 | 3-Chloro-6-furan-3-yl-8- trifluoromethyl-imidazo[1,2- a]pyridine-2-carboxylic acid (1- acetyl-azetidin-3-yl)-amide |
| 514 | 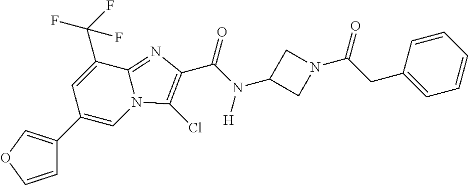 | 3-Chloro-6-furan-3-yl-8- trifluoromethyl-imidazo[1,2- a]pyridine-2-carboxylic acid (1- phenylacetyl-azetidin-3-yl)-amide |
| 515 | 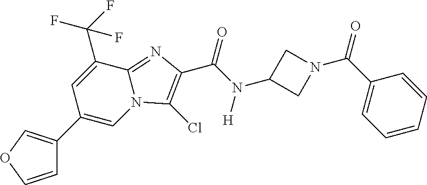 | 3-Chloro-6-furan-3-yl-8- trifluoromethyl-imidazo[1,2- a]pyridine-2-carboxylic acid (1- benzoyl-azetidin-3-yl)-amide |
| 516 | 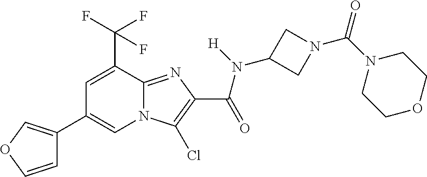 | 3-Chloro-6-furan-3-yl-8- trifluoromethyl-imidazo[1,2- a]pyridine-2-carboxylic acid [1- (morpholine-4-carbonyl)-azetidin- 3-yl]-amide |
| 517 | 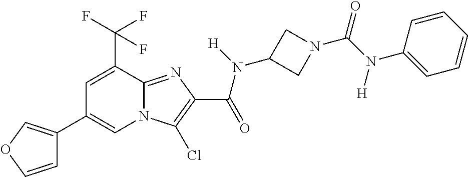 | 3-Chloro-6-furan-3-yl-8- trifluoromethyl-imidazo[1,2- a]pyridine-2-carboxylic acid (1- phenylcarbamoyl-azetidin-3-yl)- amide |
| 518 | 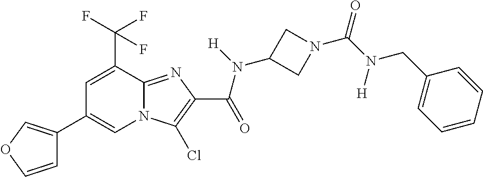 | 3-Chloro-6-furan-3-yl-8- trifluoromethyl-imidazo[1,2- a]pyridine-2-carboxylic acid (1- benzylcarbamoyl-azetidin-3-yl)- amide |
| 519 | 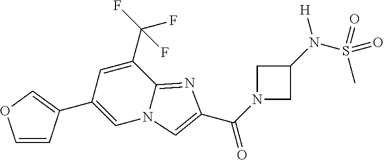 | N-(1-{[6-(furan-3-yl)-8- (trifluoromethyl)imidazo[1,2- a]pyridin-2-yl]carbonyl}azetidin- 3-yl)methanesulfonamide |
| 520 | 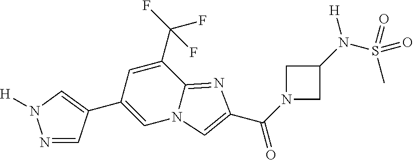 | N-(1-{[6-(1H-pyrazol-4-yl)-8- (trifluoromethyl)imidazo[1,2- a]pyridin-2-yl]carbonyl}azetidin- 3-yl)methanesulfonamide |
| 521 | 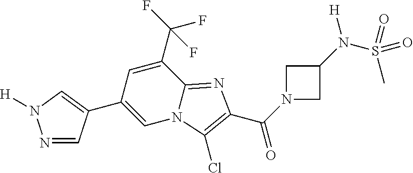 | N-(1-{[3-chloro-6-(1H-pyrazol-4- yl)-8- (trifluoromethyl)imidazo[1,2- a]pyridin-2-yl]carbonyl}azetidin- 3-yl)methanesulfonamide |
| 522 | 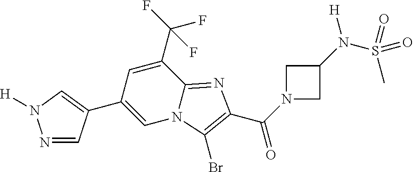 | N-(1-{[3-bromo-6-(1H-pyrazol-4- yl)-8- (trifluoromethyl)imidazo[1,2- a]pyridin-2-yl]carbonyl}azetidin- 3-yl)methanesulfonamide |
| 523 | 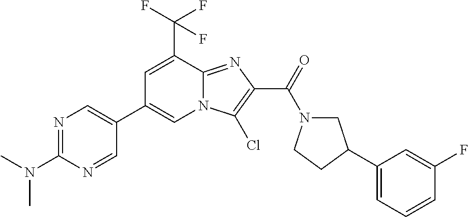 | [3-Chloro-6-(2-dimethylamino- pyrimidin-5-yl)-8-trifluoromethyl- imidazo[1,2-a]pyridin-2-yl]-[3-(3- fluoro-phenyl)-pyrrolidin-1-yl]- methanone |
| 523 | 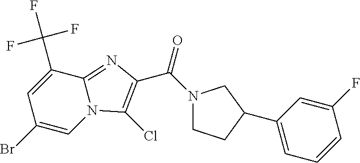 | (6-Bromo-3-chloro-8- trifluoromethyl-imidazo[1,2- a]pyridin-2-yl)-[3-(3-fluoro- phenyl)-pyrrolidin-1-yl]- methanone |
| 524 | 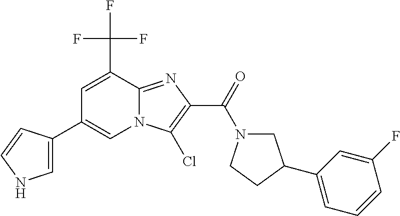 | [3-Chloro-6-(1H-pyrrol-3-yl)-8- trifluoromethyl-imidazo[1,2- a]pyridin-2-yl]-[3-(3-fluoro- phenyl)-pyrrolidin-1-yl]- methanone |
| 525 | 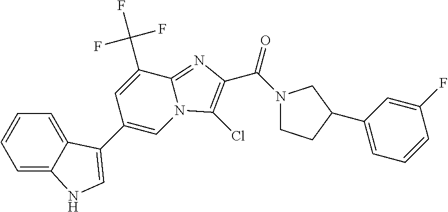 | [3-Chloro-6-(1H-indol-3-yl)-8- trifluoromethyl-imidazo[1,2- a]pyridin-2-yl]-{3-(3-fluoro- phneyl)-pyrrolidin-1-yl]- methanone |
| 526 | 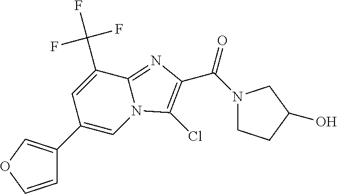 | (3-Chloro-6-furan-3-yl-8- trifluoromethyl-imidazo[1,2- a]pyridin-2-yl)-(3-hydroxy- pyrrolidin-1-yl)-methanone |
| 527 | 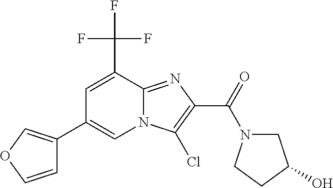 | (3-Chloro-6-furan-3-yl-8- trifluoromethyl-imidazo[1,2- a]pyridin-2-yl)-(3-(R)-hydroxy- pyrrolidin-1-yl)-methanone |
| 528 | 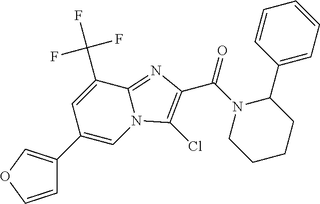 | (3-Chloro-6-furan-3-yl-8- trifluoromethyl-imidazo[1,2- a]pyridin-2-yl)-(2-phenyl- piperidin-1-yl)-methanone |
| 528 | 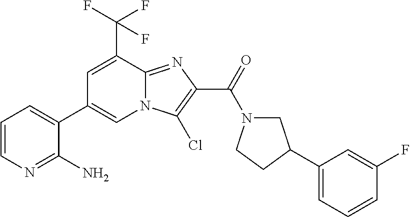 | [6-(2-Amino-pyridin-3-yl)-3- chloro-8-trifluoromethyl- imidazo[1,2-a]pyridin-2-yl]-[3-(3- fluoro-phenyl)-pyrrolidin-1-yl]- methanone |
| 529 | 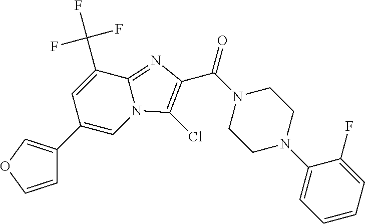 | (3-Chloro-6-furan-3-yl-8- trifluoromethyl-imidazo[1,2- a]pyridin-2-yl)-[4-(2-fluoro- phenyl)-piperazin-1-yl]- methanone |
| 530 | 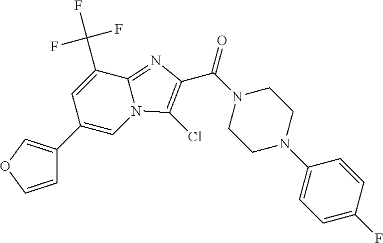 | (3-Chloro-6-furan-3-yl-8- trifluoromethyl-imidazo[1,2- a]pyridin-2-yl)-[4-(4-fluoro- phenyl)-piperazin-1-yl]- methanone |
| 531 | 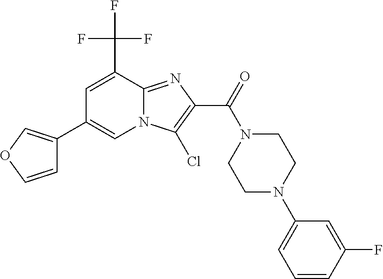 | (3-Chloro-6-furan-3-yl-8- trifluoromethyl-imidazo[1,2- a]pyridin-2-yl)-[4-(3-fluoro- phenyl)-piperazin-1-yl]- methanone |
| 532 | 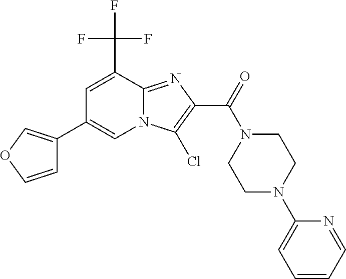 | (3-Chloro-6-furan-3-yl-8- trifluoromethyl-imidazo[1,2- a]pyridin-2-yl)-(4-pyridin-2-yl- piperazin-1-yl)-methanone |
| 533 | 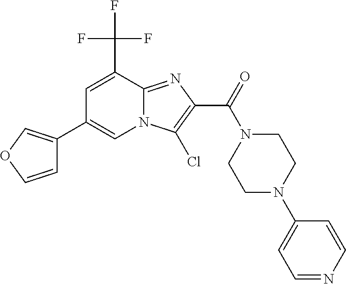 | (3-Chloro-6-furan-3-yl-8- trifluoromethyl-imidazo[1,2- a]pyridin-2-yl)-(4-pyridin-4-yl- piperazin-1-yl)-methanone |
| 534 | 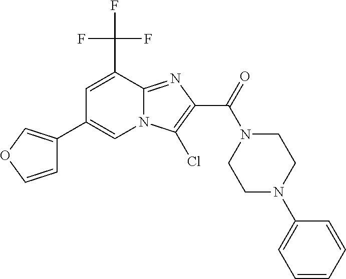 | (3-Chloro-6-furan-3-yl-8- trifluoromethyl-imidazo[1,2- a]pyridin-2-yl)-(4-phenyl- piperazin-1-yl)-methanone |
| 535 | 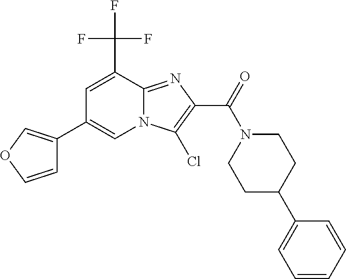 | (3-Chloro-6-furan-3-yl-8- trifluoromethyl-imidazo[1,2- a]pyridin-2-yl)-(4-phenyl- piperidin-1-yl)-methanone |
| 536 | 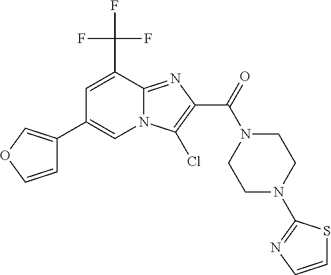 | (3-Chloro-6-furan-3-yl-8- trifluoromethyl-imidazo[1,2- a]pyridin-2-yl)-(4-thiazol-2-yl- piperazin-1-yl)-methanone |
| 537 | 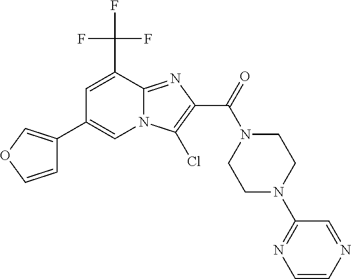 | (3-Chloro-6-furan-3-yl-8- trifluoromethyl-imidazo[1,2- a]pyridin-2-yl)-(2,3,5,6- tetrahydro-[1,2′]bipyrazinyl-4-yl)- methanone |
| 538 | 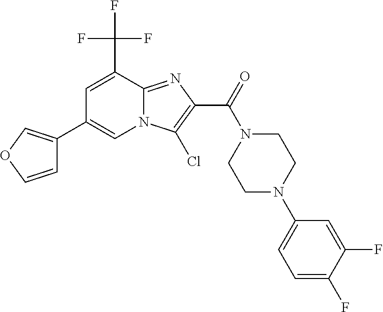 | (3-Chloro-6-furan-3-yl-8- trifluoromethyl-imidazo[1,2- a]pyridin-2-yl)-[4-(3,4-difluoro- phenyl)-piperazin-1-yl]- methanone |
| 539 | 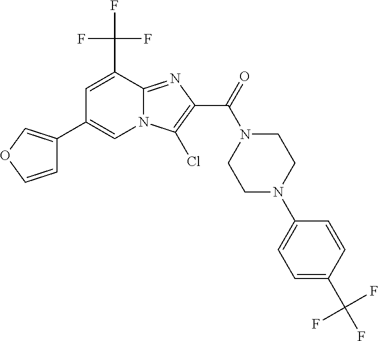 | (3-Chloro-6-furan-3-yl-8- trifluoromethyl-imidazo[1,2- a]pyridin-2-yl)-[4-(4- trifluoromethyl-phenyl)- piperazin-1-yl]-methanone |
| 540 | 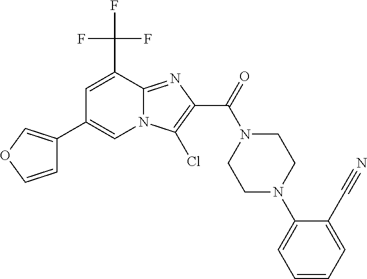 | 2-[1-(3-Chloro-6-furan-3-yl-8- trifluoromethyl-imidazo[1,2- a]pyridine-2-carbonyl)-piperidin- 4-yl]-benzonitrile |
| 541 | 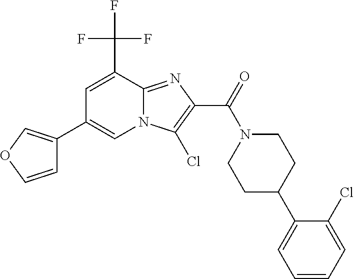 | (3-Chloro-6-furan-3-yl-8- trifluoromethyl-imidazo[1,2- a]pyridin-2-yl)-[4-(2-chloro- phenyl)-piperidin-1-yl]- methanone |
| 542 | 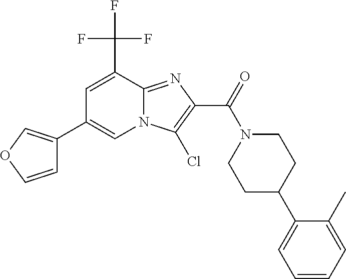 | (3-Chloro-6-furan-3-yl-8- trifluoromethyl-imidazo[1,2- a]pyridin-2-yl)-(4-o-tolyl- piperidin-1-yl)-methanone |
| 543 | 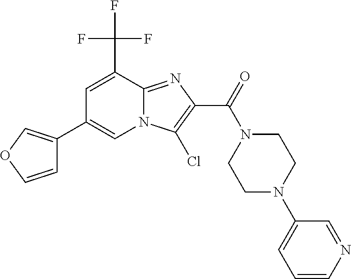 | (3-Chloro-6-furan-3-yl-8- trifluoromethyl-imidazo[1,2- a]pyridin-2-yl)-(4-pyridin-3-yl- piperazin-1-yl)-methanone |
| 544 | 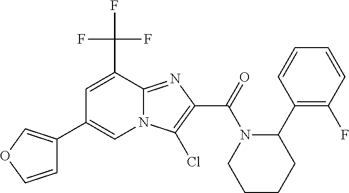 | (3-Chloro-6-furan-3-yl-8- trifluoromethyl-imidazo[1,2- a]pyridin-2-yl)-[2-(2-fluoro- phenyl)-piperidin-1-yl]- methanone |
| 545 | 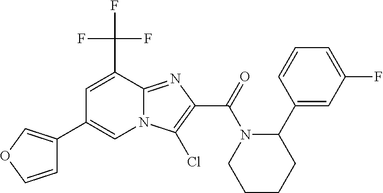 | (3-Chloro-6-furan-3-yl-8- trifluoromethyl-imidazo[1,2- a]pyridin-2-yl)-[2-(3-fluoro- phenyl)-piperidin-1-yl]- methanone |
| 546 | 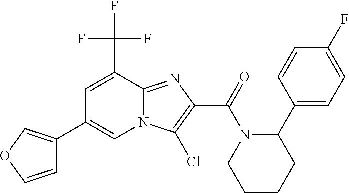 | (3-Chloro-6-furan-3-yl-8- trifluoromethyl-imidazo[1,2- a]pyridin-2-yl)-[2-(4-fluoro- phenyl)-piperidin-1-yl]- methanone |
| 547 | 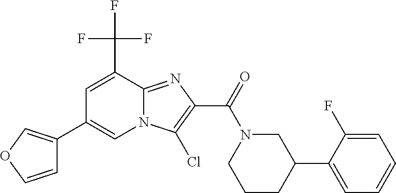 | (3-Chloro-6-furan-3-yl-8- trifluoromethyl-imidazo[1,2- a]pyridin-2-yl)-[3-(2-fluoro- phenyl)-piperidin-1-yl]- methanone |
| 548 | 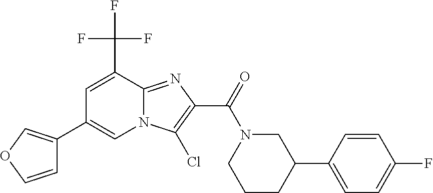 | (3-Chloro-6-furan-3-yl-8- trifluoromethyl-imidazo[1,2- a]pyridin-2-yl)-[3-(4-fluoro- phenyl)-piperidin-1-yl]- methanone |
| 549 | 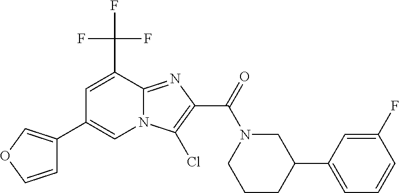 | (3-Chloro-6-furan-3-yl-8- trifluoromethyl-imidazo[1,2- a]pyridin-2-yl)-[3-(3-fluoro- phenyl)-piperidin-1-yl]- methanone |
| 550 | 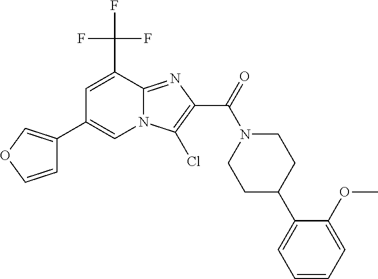 | (3-Chloro-6-furan-3-yl-8- trifluoromethyl-imidazo[1,2- a]pyridin-2-yl)-[4-(2-methoxy- phenyl)-piperidin-1-yl]- methanone |
| 551 | 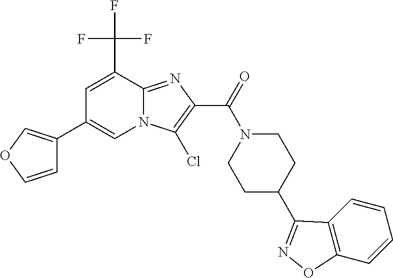 | (4-Benzo[d]isoxazol-3-yl- piperazin-1-yl)-(3-chloro-6-furan- 3-yl-8-trifluoromethyl- imidazo[1,2-a]pyridin-2-yl)- methanone |
| 552 | 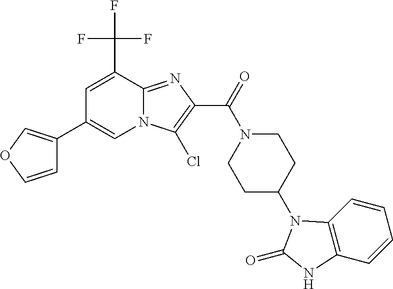 | 1-[1-(3-Chloro-6-furan-3-yl-8- trifluoromethyl-imidazo[1,2- a]pyridine-2-carbonyl)-piperidin- 4-yl]-1,3-dihydro-benzoimidazol- 2-one |
| 553 | 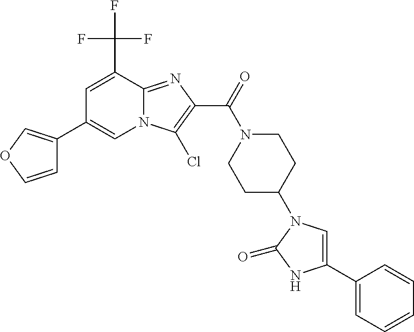 | 1-[1-(3-Chloro-6-furan-3-yl-8- trifluoromethyl-imidazo[1,2- a]pyridine-2-carbonyl)-piperidin- 4-yl]-4-phenyl-1,3-dihydro- imidazol-2-one |
| 554 | 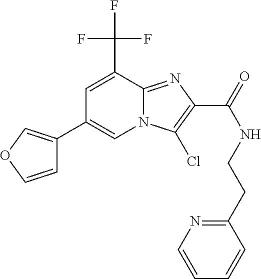 | 3-chloro-6-(furan-3-yl)-N-[2- (pyridin-2-yl)ethyl]-8- (trifluoromethyl)imidazo[1,2- a]pyridine-2-carboxamide |
| 555 | 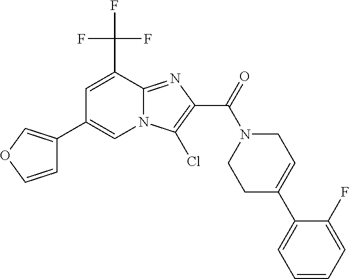 | (3-Chloro-6-furan-3-yl-8- trifluoromethyl-imidazo[1,2- a]pyridin-2-yl)-[4-(2-fluoro- phenyl)-3,6-dihydro-2H-pyridin- 1-yl]-methanone |
| 556 | 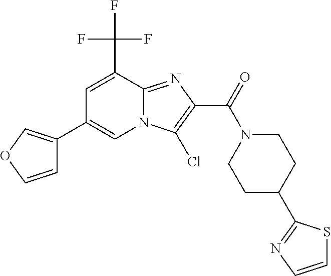 | (3-Chloro-6-furan-3-yl-8- trifluoromethyl-imidazo[1,2- a]pyridin-2-yl)-(4-thiazol-2-yl- piperidin-1-yl)-methanone |
| 557 | 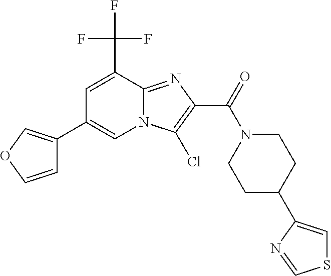 | (3-Chloro-6-furan-3-yl-8- trifluoromethyl-imidazo[1,2- a]pyridin-2-yl)-(4-thiazol-4-yl- piperidin-1-yl)-methanone |
| 558 | 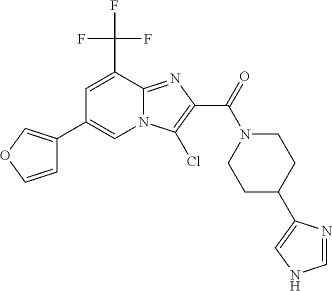 | [3-chloro-6-(furan-3-yl)-8- (trifluoromethyl)imidazo[1,2- a]pyridin-2-yl][4-(1H-imidazol-4- yl)-3,6-dihydropyridin-1(2H)- yl]methanone |
| 559 | 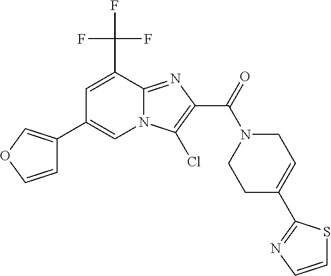 | (3-Chloro-6-furan-3-yl-8- trifluoromethyl-imidazo[1,2- a]pyridin-2-yl)-(4-thiazol-2-yl- 3,6-dihydro-2H-pyridin-1-yl)- methanone |
| 560 | 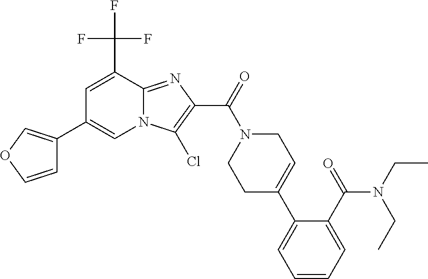 | 2-[1-(3-Chloro-6-furan-3-yl-8- trifluoromethyl-imidazo[1,2- a]pyridine-2-carbonyl)-1,2,3,6- tetrahydro-pyridin-4-yl]-N,N- diethyl-benzamide |
| 561 | 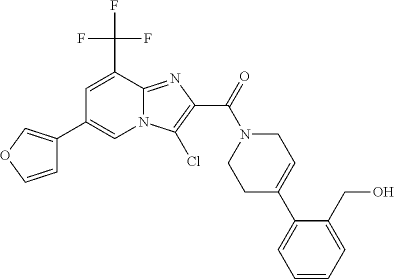 | (3-Chloro-6-furan-3-yl-8- trifluoromethyl-imidazo[1,2- a]pyridin-2-yl)-[4-(2- hydroxymethyl-phenyl)-3,6- dihydro-2H-pyridin-1-yl]- methanone |
| 562 | 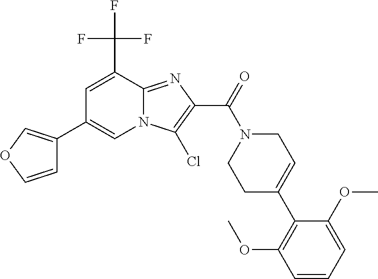 | (3-Chloro-6-furan-3-yl-8- trifluoromethyl-imidazo[1,2- a]pyridin-2-yl)-[4-(2,6- dimethoxy-phenyl)-3,6-dihydro- 2H-pyridin-1-yl]-methanone |
| 563 | 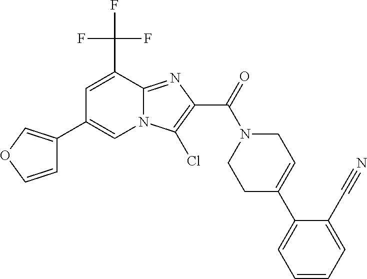 | 2-[1-(3-Chloro-6-furan-3-yl-8- trifluoromethyl-imidazo[1,2- a]pyridine-2-carbonyl)-1,2,3,6- tetrahydro-pyridin-4-yl]- benzonitrile |
| 564 | 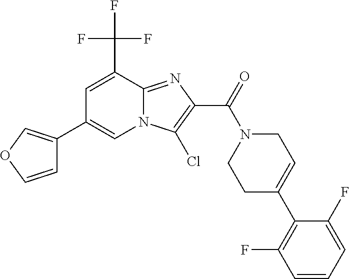 | (3-Chloro-6-furan-3-yl-8- trifluoromethyl-imidazo[1,2- a]pyridin-2-yl)-[4-(2,6-difluoro- phenyl)-3,6-dihydro-2H-pyridin- 1-yl]-methanone |
| 565 | 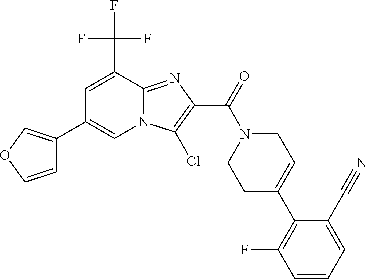 | 2-[1-(3-Chloro-6-furan-3-yl-8- trifluoromethyl-imidazo[1,2- a]pyridine-2-carbonyl)-1,2,3,6- tetrahydro-pyridin-4-yl]-3-fluroo- benzonitrile |
| 566 | 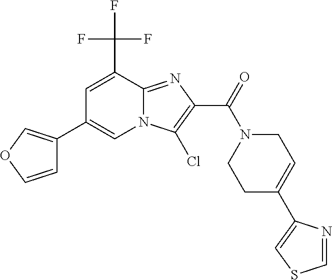 | (3-Chloro-6-furan-3-yl-8- trifluoromethyl-imidazo[1,2- a]pyridin-2-yl)-(4-thiazol-4-yl- 3,6-dihydro-2H-pyridin-1-yl)- methanone |
| 567 | 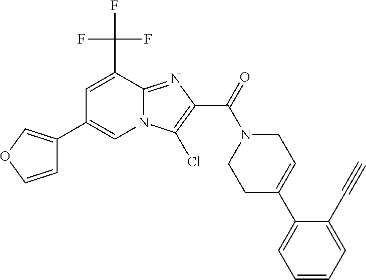 | (3-Chloro-6-furan-3-yl-8- trifluoromethyl-imidazo[1,2- a]pyridin-2-yl)-[4-(2-ethynyl- phenyl)-3,6-dihydro-2H-pyridin- 1-yl]-methanone |
| 568 | 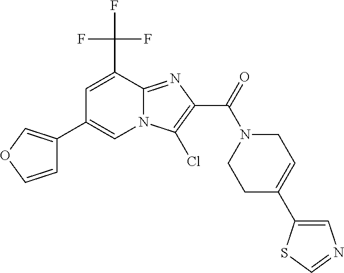 | (3-Chloro-6-furan-3-yl-8- trifluoromethyl-imidazo[1,2- a]pyridin-2-yl)-(4-thiazol-5-yl- 3,6-dihydro-2H-pyridin-1-yl)- methanone |
| 569 | 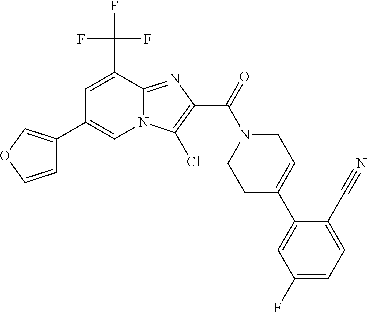 | 2-[1-(3-Chloro-6-furan-3-yl-8- trifluoromethyl-imidazo[1,2- a]pyridine-2-carbonyl)-1,2,3,6- tetrahydro-pyridin-4-yl]-4-fluroo- benzonitrile |
| 570 | 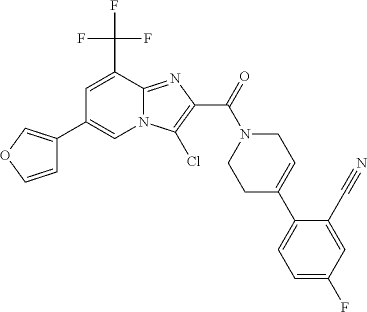 | 2-[1-(3-Chloro-6-furan-3-yl-8- trifluoromethyl-imidazo[1,2- a]pyridine-2-carbonyl)-1,2,3,6- tetrahydro-pyridin-4-yl]-5-fluoro- benzonitrile |
| 571 | 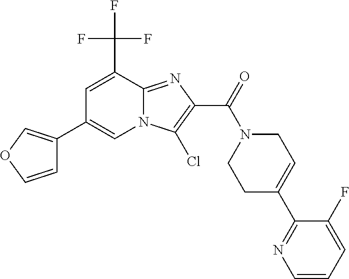 | [3-chloro-6-(furan-3-yl)-8- (trifluoromethyl)imidazo[1,2- a]pyridin-2-yl](3-fluoro-3′,6′- dihydro-2,4′-bipyridin-1′(2′H)- yl)methanone |
| 572 | 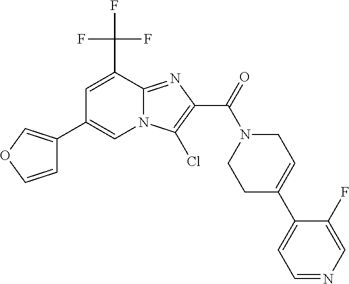 | [3-chloro-6-(furan-3-yl)-8- (trifluoromethyl)imidazo[1,2- a]pyridin-2-yl](3′-fluoro-3,6- dihydro-4,4′-bipyridin-1(2H)- yl)methanone |
| 573 | 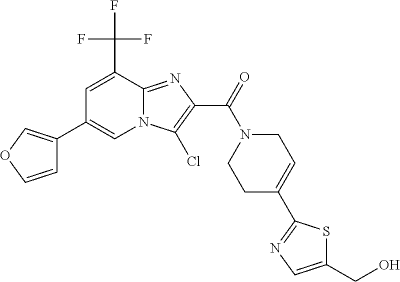 | [3-chloro-6-(furan-3-yl)-8- (trifluoromethyl)imidazo[1,2- a]pyridin-2-yl]{4-[5- (hydroxymethyl)-1,3-thiazol-2- yl]-3,6-dihydropyridin-1(2H)- yl}methanone |
| 574 | 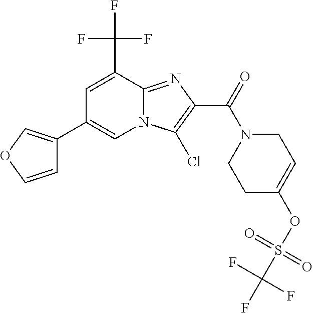 | Trifluoro-methanesulfonic acid 1- (3-chloro-6-furan-3-yl-8- trifluoromethyl-imidazo[1,2- a]pyridine-2-carbonyl)-1,2,3,6- tetrahydro-pyridin-4-yl ester |
| 575 | 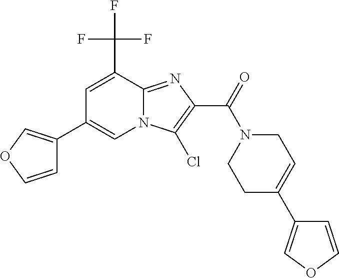 | (3-Chloro-6-furan-3-yl-8- trifluoromethyl-imidazo[1,2- a]pyridin-2-yl)-(4-furan-3-yl-3,6- dihydro-2H-pyridin-1-yl)- methanone |
| 576 | 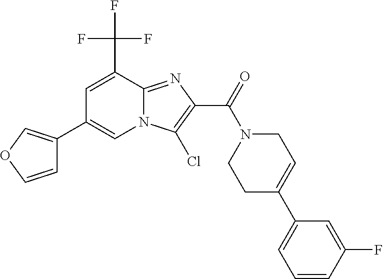 | (3-Chloro-6-furan-3-yl-8- trifluoromethyl-imidazo[1,2- a]pyridin-2-yl)-[4-(3-fluoro- phenyl)-3,6-dihydro-2H-pyridin- 1-yl]-methanone |
| 577 | 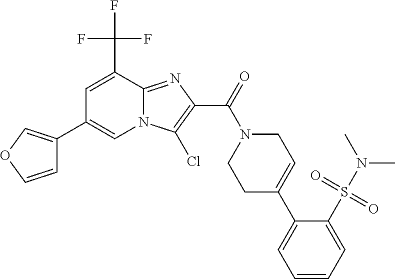 | 2-[1-(3-Chloro-6-furan-3-yl-8- trifluoromethyl-imidazo[1,2- a]pyridine-2-carbonyl)-1,2,3,6- tetrahydro-pyridin-4-yl]-N,N- dimethyl-benzenesulfonamide |
| 578 | 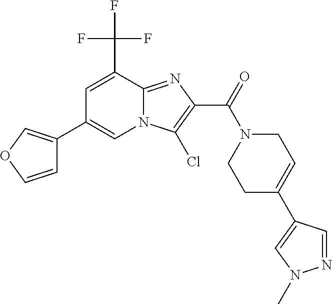 | (3-Chloro-6-furan-3-yl-8- trifluoromethyl-imidazo[1,2- a]pyridin-2-yl)-[4-(1-methyl-1H- pyrazol-4-yl)-3,6-dihydro-2H- pyridin-1-yl]-methanone |
| 579 | 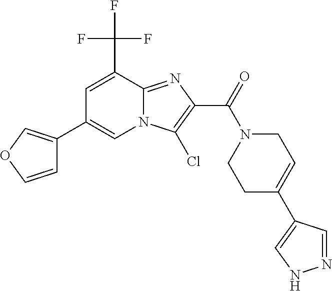 | (3-Chloro-6-furan-3-yl-8- trifluoromethyl-imidazo[1,2- a]pyridin-2-yl)-[4-(1H-pyrazol-4- yl)-3,6-dihydro-2H-pyridin-1-yl]- methanone |
| 580 | 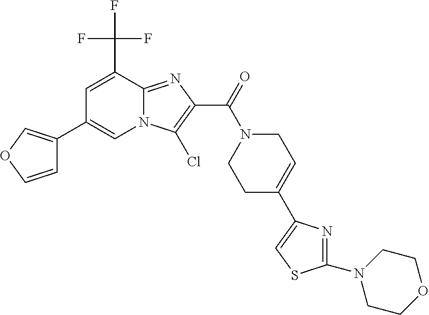 | (3-Chloro-6-furan-3-yl-8- trifluoromethyl-imidazo[1,2- a]pyridin-2-yl)-[4-(2-morpholin- 4-yl-thiazol-4-yl)-3,6-dihydro- 2H-pyridin-1-yl]-methanone |
| 581 | 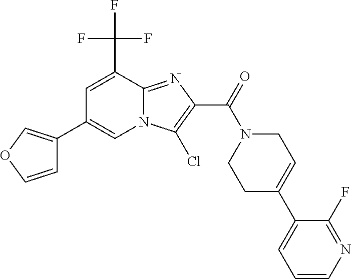 | (3-Chloro-6-furan-3-yl-8- trifluoromethyl-imidazo[1,2- a]pyridin-2-yl)-(2-fluoro-3′,6′- dihydro-2′H-[3,4′]bipyridinyl-1′- yl)-methanone |
| 582 | 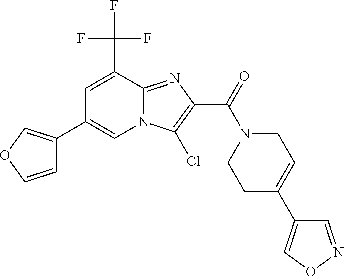 | (3-Chloro-6-furan-3-yl-8- trifluoromethyl-imidazo[1,2- a]pyridin-2-yl)-(4-isoxazol-4-yl- 3,6-dihydro-2H-pyridin-1-yl)- methanone |
| 583 | 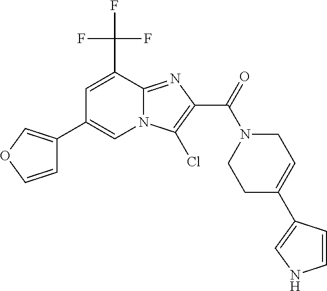 | [3-chloro-6-(furan-3-yl)-8- (trifluoromethyl)imidazo[1,2- a]pyridin-2-yl][4-(1H-pyrrol-3- yl)-3,6-dihydro-pyridin-1(2H)- yl]methanone |
| 584 | 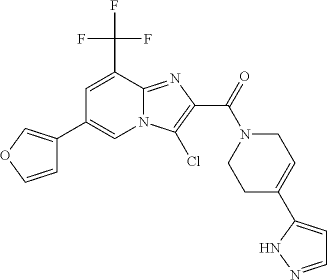 | [3-chloro-6-(furan-3-yl)-8- (trifluoromethyl)imidazo[1,2- a]pyridin-2-yl][4-(1H-pyrazol-5- yl)-3,6-dihydro-pyridin-1(2H)- yl]methanone |
| 585 | 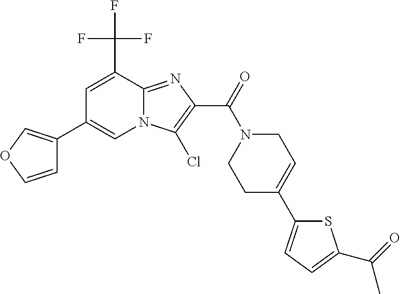 | 1-[5-(1-{[3-chloro-6-(furan-3-yl)- 8-(trifluoromethyl)imidazo[1,2- a]pyridin-2-yl]carbonyl}-1,2,3,6- tetrahydropyridin-4-yl)thiophen- 2-yl]ethanone |
| 586 | 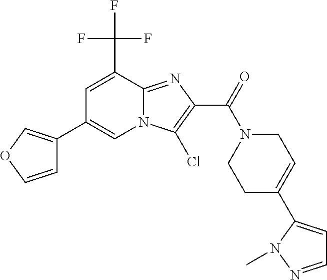 | [3-chloro-6-(furan-3-yl)-8- (trifluoromethyl)imidazo[1,2- a]pyridin-2-yl][4-(1-methyl-1H- pyrazol-5-yl)-3,6-dihydropyridin- 1(2H)-yl]methanone |
| 587 | 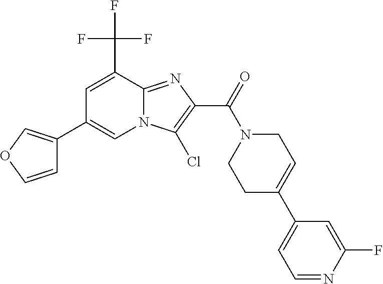 | [3-chloro-6-(furan-3-yl)-8- (trifluoromethyl)imidazo[1,2- a]pyridin-2-yl](2′-fluoro-3,6- dihydro-4,4′-bipyridin-1(2H)- yl)methanone |
| 588 | 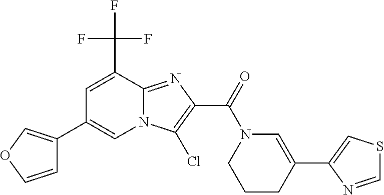 | [3-chloro-6-(furan-3-yl)-8- (trifluoromethyl)imidazo[1,2- a]pyridin-2-yl][5-(1,3-thiazol-4- yl)-3,4-dihydro-pyridin-1(2H)- yl]methanone |
| 589 | 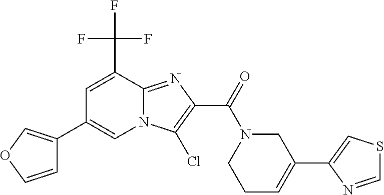 | [3-chloro-6-(furan-3-yl)-8- (trifluoromethyl)imidazo[1,2- a]pyridin-2-yl][5-(1,3-thiazol-4- yl)-3,6-dihydropyridin-1(2H)- yl]methanone |
| 590 | 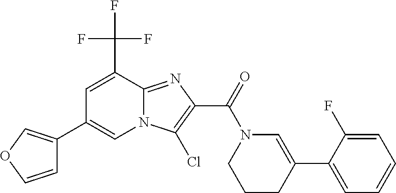 | (3-Chloro-6-furan-3-yl-8- trifluoromethyl-imidazo[1,2- a]pyridin-2-yl)-[5-(2-fluoro- phenyl)-3,4-dihydro-2H-pyridin- 1-yl]-methanone |
| 591 | 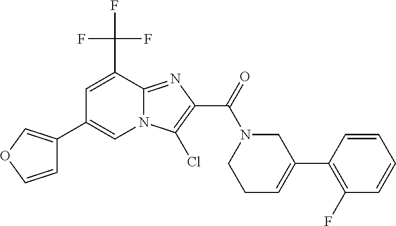 | (3-Chloro-6-furan-3-yl-8- trifluoromethyl-imidazo[1,2- a]pyridin-2-yl)-[5-(2-fluoro phenyl)-3,6-dihydro-2H-pyridin- 1-yl]-methanone |
| 592 | 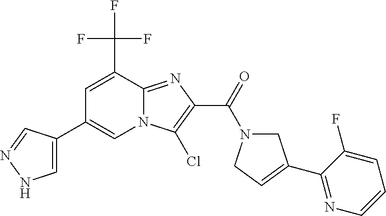 | [3-chloro-6-(1H-pyrazol-4-yl)-8- (trifluoromethyl)imidazo[1,2- a]pyridin-2-yl][3-(3- fluoropyridin-2-yl)-2,5-dihydro- 1H-pyrrol-1-yl]methanone |
| 593 | 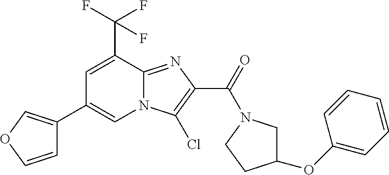 | (3-Chloro-6-furan-3-yl-8- trifluoromethyl-imidazo[1,2- a]pyridin-2-yl)-(3-phenoxy- pyrrolidin-1-yl)-methanone |
| 594 | 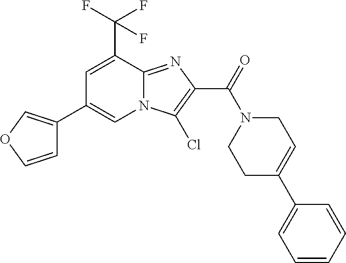 | (3-Chloro-6-furan-3-yl-8- trifluoromethyl-imidazo[1,2- a]pyridin-2-yl)-(4-phenyl-3,6- dihydro-2H-pyridin-1-yl)- methanone |
| 595 | 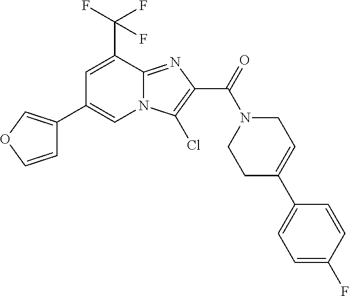 | (3-Chloro-6-furan-3-yl-8- trifluoromethyl-imidazo[1,2- a]pyridin-2-yl)-[4-(4-fluoro- phenyl)-3,6-dihydro-2H-pyridin- 1-yl]-methanone |
| 596 | 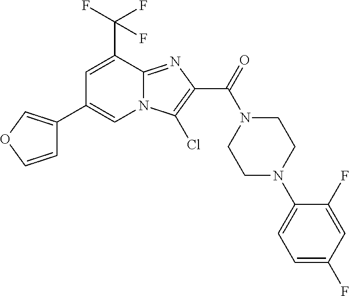 | (3-Chloro-6-furan-3-yl-8- trifluoromethyl-imidazo[1,2- a]pyridin-2-yl)-[4-(2,4-difluoro- phenyl)-piperazin-1-yl]- methanone |
| 597 | 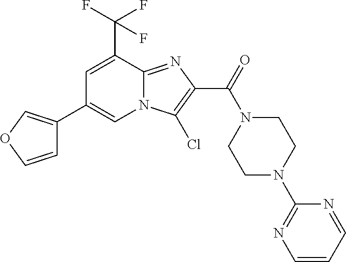 | (3-Chloro-6-furan-3-yl-8- trifluoromethyl-imidazo[1,2- a]pyridin-2-yl)-(4-pyrimidin-2-yl- piperazin-1-yl)-methanone |
| 598 | 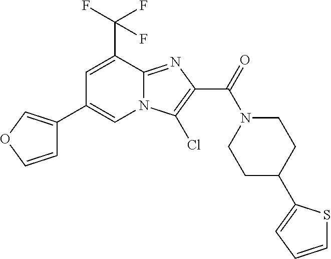 | [3-chloro-6-(furan-3-yl)-8- (trifluoromethyl)imidazo[1,2- a]pyridin-2-yl][4-(thiophen-2- yl)piperidin-1-yl]methanone |
| 599 | 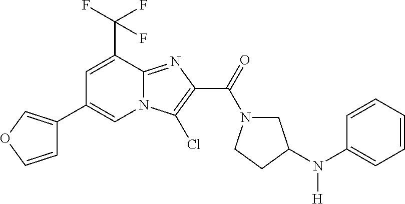 | (3-Chloro-6-furan-3-yl-8- trifluoromethyl-imidazo[1,2- a]pyridin-2-yl)-(3-phenylamino- pyrrolidin-1-yl)-methanone |
| 600 | 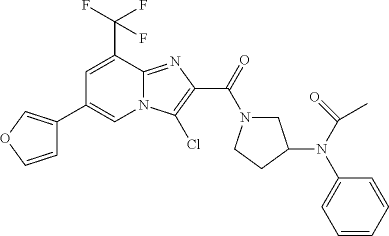 | N-[1-(3-Chloro-6-furan-3-yl-8- trifluoromethyl-imidazo[1,2- a]pyridine-2-carbonyl)-pyrrolidin- 3-yl]-N-phenyl-acetamide |
| 601 | 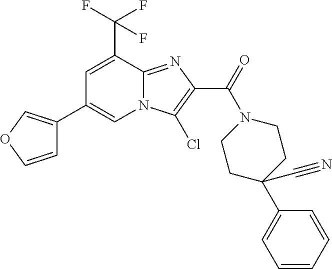 | 1-(3-Chloro-6-furan-3-yl-8- trifluoromethyl-imidazo[1,2- a]pyridine-2-carbonyl)-4-phenyl- piperidine-4-carbonitrile |
| 602 | 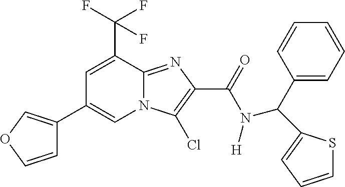 | 3-Chloro-6-furan-3-yl-8- trifluoromethyl-imidazo[1,2- a]pyridine-2-carboxylic acid (phenyl-thiophen-2-yl-methyl)- amide |
| 603 | 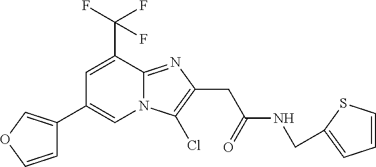 | 2-[3-chloro-6-(furan-3-yl)-8- (trifluoromethyl)imidazo[1,2- a]pyridin-2-yl]-N-(thiophen-2- ylmethyl)acetamide |
| 604 | 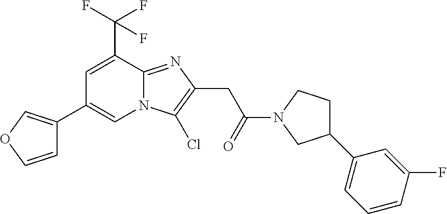 | 2-[3-chloro-6-(furan-3-yl)-8- (trifluoromethyl)imidazo[1,2- a]pyridin-2-yl]-1-[3-(3- fluorophenyl)pyrrolidin-1- yl]ethanone |
| 605 | 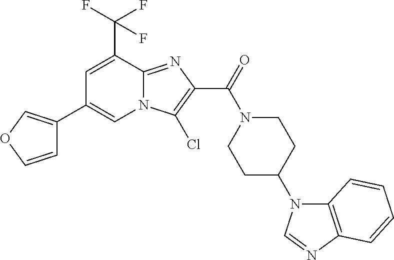 | (4-Benzoimidazol-1-yl-piperidin- 1-yl)-(3-chloro-6-furan-3-yl-8- trifluoromethyl-imidazo[1,2- a]pyridin-2-yl)-methanone |
| 606 | 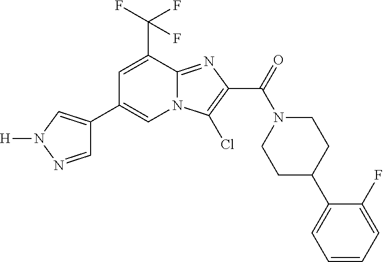 | [3-Chloro-6-(1H-pyrazol-4-yl)-8- trifluoromethyl-imidazo[1,2- a]pyridin-2-yl]-[4-(2-fluoro- phenyl)-piperidin-1-yl]- methanone |
| 607 | 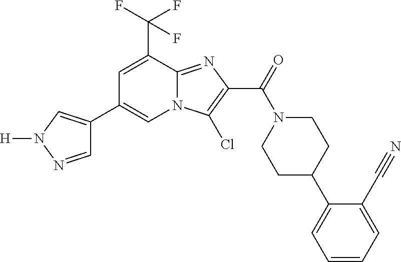 | 2-{1-[3-Chloro-6-(1H-pyrazol-4- yl)-8-trifluoromethyl- imidazo[1,2-a]pyridine-2- carbonyl]-piperidin-4-yl}- benzonitrile |
| 608 | 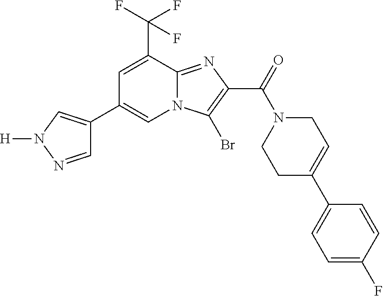 | [3-Bromo-6-(1H-pyraozl-4-yl)-8- trifluoromethyl-imidazo[1,2- a]pyridin-2-yl]-[4-(4-fluoro- phenyl)-3,6-dihydro-2H-pyridin- 1-yl]-methanone |
| 609 | 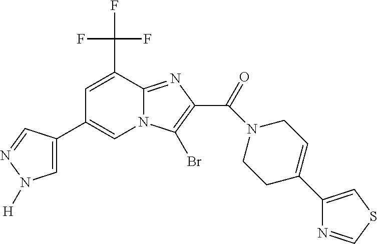 | [3-Bromo-6-(1H-pyrazol-4-yl)-8- trifluoromethyl-imidazo[1,2- a]pyridin-2-yl]-(4-thiazol-4-yl- 3,6-dihydro-2H-pyridin-1-yl)- methanone |
| 610 | 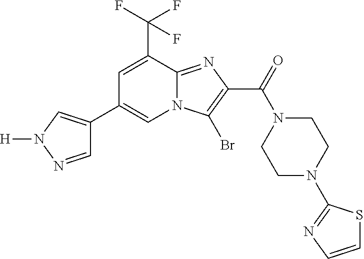 | [3-Bromo-6-(1H-pyrazol-4-yl)-8- trifluoromethyl-imidazo[1,2- a]pyridin-2-yl]-(4-thiazol-2-yl- piperazin-1-yl)-methanone |
| 611 | 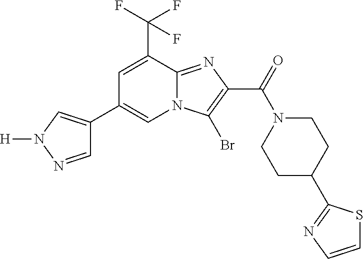 | [3-Bromo-6-(1H-pyrazol-4-yl)-8- trifluoromethyl-imidazo[1,2- a]pyridin-2-yl]-(4-thiazol-2-yl- piperidin-1-yl)-methanone |
| 612 | 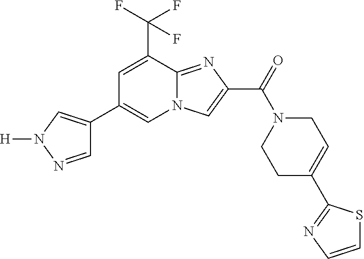 | [6-(1H-pyrazol-4-yl)-8- (trifluoromethyl)imidazo[1,2- a]pyridin-2-yl][4-(1,3-thiazol-2- yl)-3,6-dihydropyridin-1(2H)- yl]methanone |
| 613 | 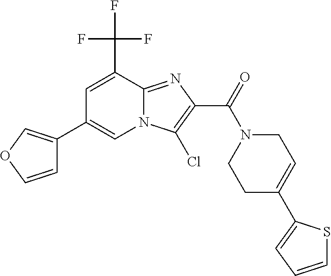 | (3-Chloro-6-furan-3-yl-8- trifluoromethyl-imidazo[1,2- a]pyridin-2-yl)-(4-thiophen-2-yl- 3,6-dihydro-2H-pyridin-1-yl)- methanone |
| 614 | 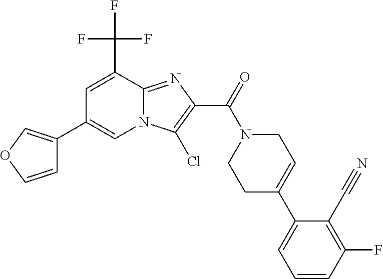 | 2-[1-(3-Chloro-6-furan-3-yl-8- trifluoromethyl-imidazo[1,2- a]pyridine-2-carbonyl)-1,2,3,6- tetrahydro-pyridin-4-yl]-6-fluoro- benzonitrile |
| 615 | 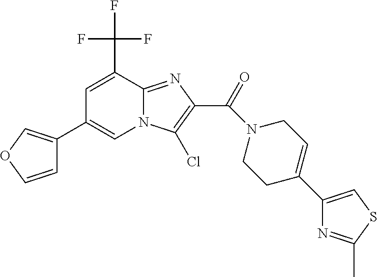 | (3-Chloro-6-furan-3-yl-8- trifluoromethyl-imidazo[1,2- a]pyridin-2-yl)-[4-(2-methyl- thiazol-4-yl)-3,6-dihydro-2H- pyridin-1-yl]-methanone |
| 616 | 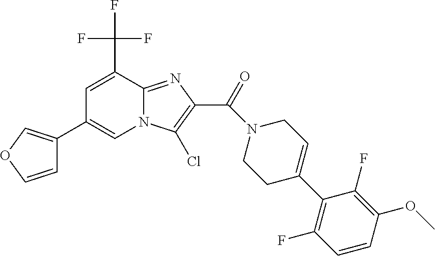 | (3-Chloro-6-furan-3-yl-8- trifluoromethyl-imidazo[1,2- a]pyridin-2-yl)-[4-(2,6-difluoro-3- methoxy-phenyl)-3,6-dihydro-2H- pyridin-1-yl]-methanone |
| 617 | 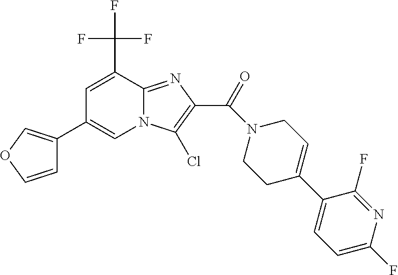 | (3-Chloro-6-furan-3-yl-8- trifluoromethyl-imidazo[1,2- a]pyridin-2-yl)-2,6-difluoro-3′,6′- dihydro-2′H-[3,4′]bipyridinyl-1′- yl)-methanone |
| 618 | 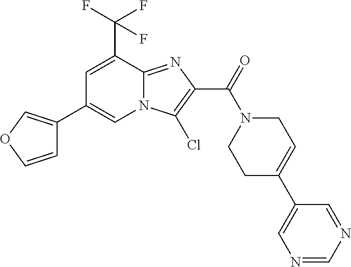 | [3-chloro-6-(furan-3-yl)-8- (trifluoromethyl)imidazo[1,2- a]pyridin-2-yl][4-(pyrimidin-5- l)-3,6-dihydro-pyridin-1(2H)- yl]methanone |
| 619 | 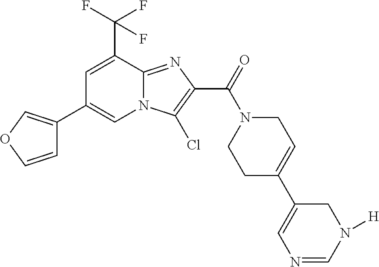 | [3-chloro-6-(furan-3-yl)-8- (trifluoromethyl)imidazo[1,2- a]pyridin-2-yl][4-(1,6- dihydropyrimidin-5-yl)-3,6- dihydropyridin-1(2H)- yl]methanone |
| 620 | 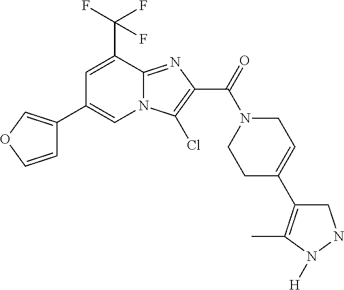 | [3-chloro-6-(furan-3-yl)-8- (trifluoromethyl)imidazo[1,2- a]pyridin-2-yl][4-(5-methyl-1H- pyrazol-4-yl)-3,6-dihydropyridin- 1(2H)-yl]methanone |
| 621 | 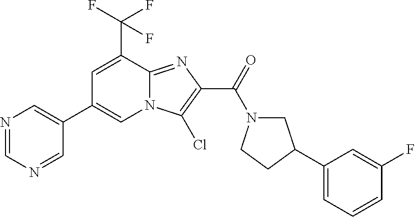 | (3-Chloro-6-pyrimidin-5-yl-8- trifluoromethyl-imidazo[1,2- a]pyridin-2-yl)-[3-(3-fluoro- phenyl)-pyrrolidin-1-yl]- methanone |
| 622 | 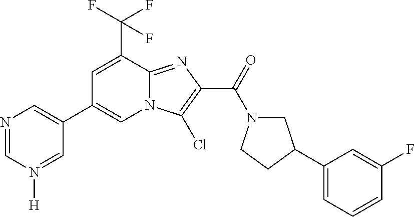 | [3-Chloro-6-(1,6-dihydro- pyrimidin-5-yl)-8-trifluoromethyl- imidazo[1,2-a]pyridin-2-yl]-[3-(3- fluoro-phenyl)-pyrrolidin-1-yl]- methanone |
| 624 | 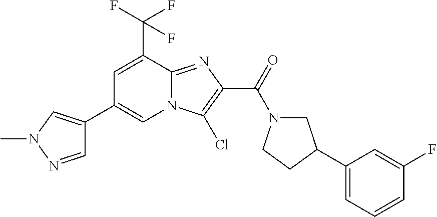 | [3-Chloro-6-(1-methyl-1H- pyrazol-4-yl)-8-trifluoromethyl- imidazo[1,2-a]pyridin-2-yl]-[3-(3- fluoro-phenyl)-pyrrolidin-1-yl]- methanone |
| 625 | 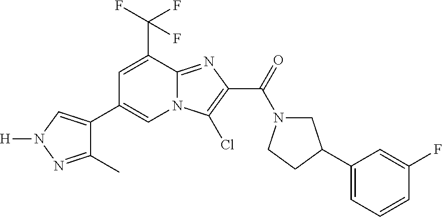 | [3-Chloro-6-(3-methyl-1H- pyrazol-4-yl)-8-trifluoromethyl- imidazo[1,2-a]pyridin-2-yl]-[3-(3- fluoro-phenyl)-pyrrolidin-1-yl]- methanone |
| 626 | 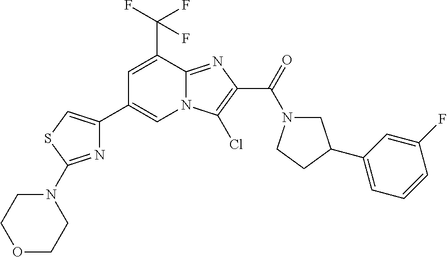 | [3-Chloro-6-(2-morpholin-4-yl- thiazol-4-yl)-8-trifluoromethyl- imidazo[1,2-a]pyridin-2-yl]-[3-(3- fluoro-phenyl)-pyrrolidin-1-yl]- methanone |
| 627 | 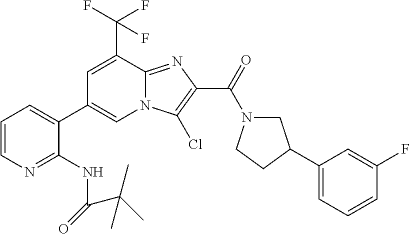 | N-(3-{3-Chloro-2-[3-(3-fluoro- phenyl)-pyrrolidine-1-carbonyl]- 8-trifluoromethyl-imidazo[1,2- a]pyridin-6-yl}-pyridin-2-yl)-2,2- dimethyl-propionamide |
| 629 | 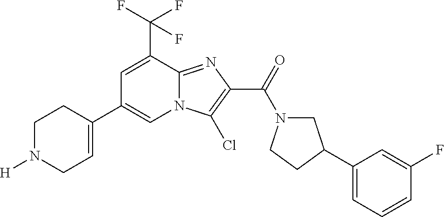 | [3-chloro-6-(1,2,3,6- tetrahydropyridin-4-yl)-8- (trifluoromethyl)imidazo[1,2- a]pyridin-2-yl][3-(3- fluorophenyl)pyrrolidin-1- yl]methanone |
| 630 | 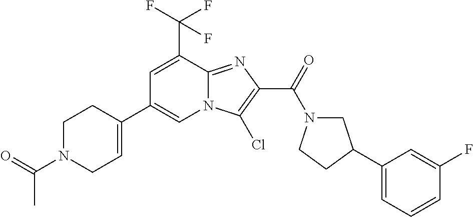 | 1-{4-[3-chloro-2-{[3-(3- fluorophenyl)pyrrolidin-1- yl]carbonyl}-8- (trifluoromethyl)imidazo[1,2- a]pyridin-6-yl]-3,6- dihydropyridin-1(2H)- yl}ethanone |
| 631 | 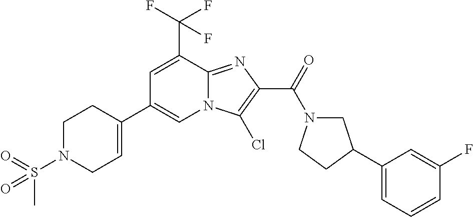 | {3-chloro-6-[1-(methylsulfonyl)- 1,2,3,6-tetrahydropyridin-4-yl]-8- (trifluoromethyl)imidazo[1,2- a]pyridin-2-yl}[3-(3- fluorophenyl)pyrrolidin-1- yl]methanone |
| 632 | 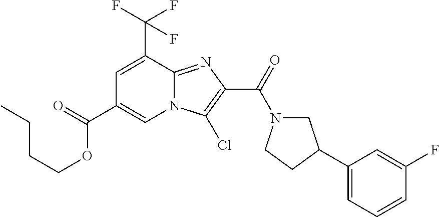 | 3-Chloro-2-[3-(3-fluoro-phenyl)- pyrrolidine-1-carbonyl]-8- trifluoromethyl-imidazo[1,2- a]pyridine-6-carboxylic acid butyl ester |
| 633 | 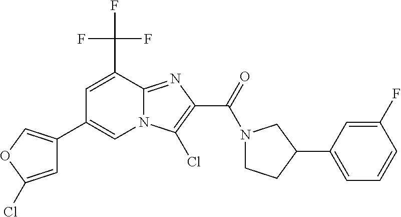 | [3-Chloro-6-(5-chloro-furan-3- yl)-8-trifluoromethyl- imidazo[1,2-a]pyridin-2-yl]-[3-(3- fluoro-phenyl)-pyrrolidin-1-yl]- methanone |
| 634 | 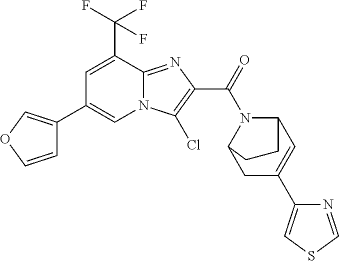 | [3-chloro-6-(furan-3-yl)-8- (trifluoromethyl)imidazo[1,2- a]pyridin-2-yl][3-(1,3-thiazol-4- yl)-8-azabicyclo[3.2.1]oct-2-en-8- yl]methanone |
| 635 | 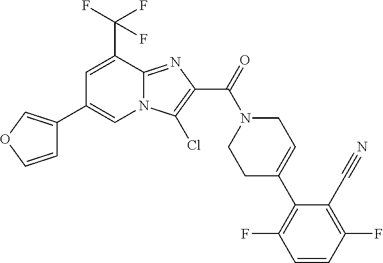 | 2-(1-{[3-chloro-6-(furan-3-yl)-8- (trifluoromethyl)imidazo[1,2- a]pyridin-2-yl]carbonyl}-1,2,3,6- tetrahydropyridin-4-yl)-3,6- difluorobenzonitrile |
| 636 | 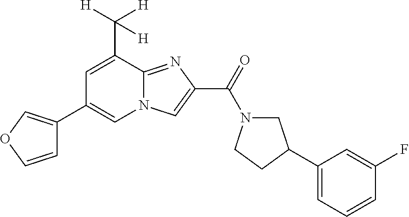 | [3-(3-Fluoro-phenyl)-pyrrolidin- 1-yl]-(6-furan-3-yl-8-methyl- imidazo[1,2-a]pyridin-2-yl)- methanone |
| 637 | 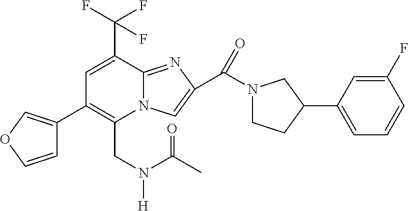 | N-{[2-{[3-(3- fluorophenyl)pyrrolidin-1- yl]carbonyl}-6-(furan-3-yl)-8- (trifluoromethyl)imidazo[1,2- a]pyridin-5-yl]methyl}acetamide |
| 638 | 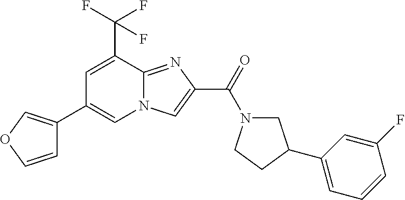 | [3-(3-Fluoro-phenyl)-pyrrolidin- 1-yl]-(6-furan-3-yl-8- trifluoromethyl-imidazo[1,2- a]pyridin-2-yl)-methanone |
| 639 | 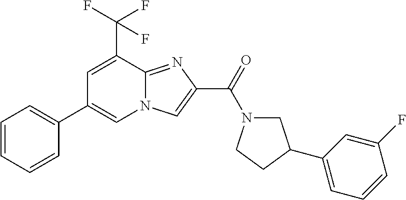 | [3-(3-Fluoro-phenyl)-pyrrolidin- 1-yl]-(6-phenyl-8-trifluoromethyl- imidazo[1,2-a]pyridin-2-yl)- methanone |
| 640 | 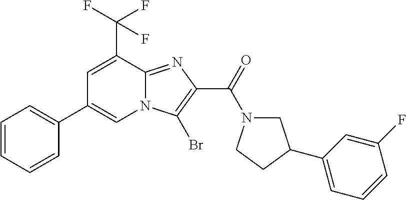 | (3-Bromo-6-phenyl-8- trifluoromethyl-imidazo[1,2- a]pyridin-2-yl)-[3-(3-fluoro- phenyl)-pyrrolidin-1-yl]- methanone |
| 641 | 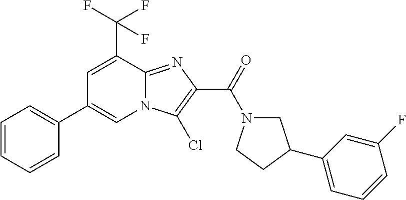 | (3-Chloro-6-phenyl-8- trifluoromethyl-imidazo[1,2- a]pyridin-2-yl)-[3-(3-fluoro- phenyl)-pyrrolidin-1-yl]- methanone |
| 642 | 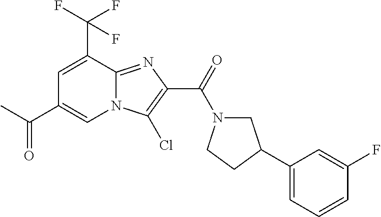 | 1-{3-Chloro-2-[3-(3-fluoro- phenyl)-pyrrolidine-1-carbonyl]- 8-trifluoromethyl-imidazo[1,2- a]pyridin-6-yl}-ethanone |
| 643 | 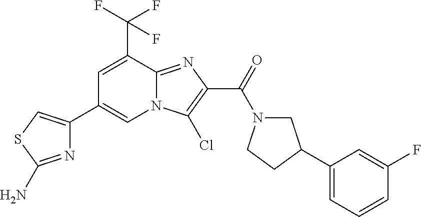 | [6-(2-Amino-thiazol-4-yl)-3- chloro-8-trifluoromethyl- imidazo[1,2-a]pyridin-2-yl]-[3-(3- fluoro-phenyl)-pyrrolidin-1-yl]- methanone |
| 644 | 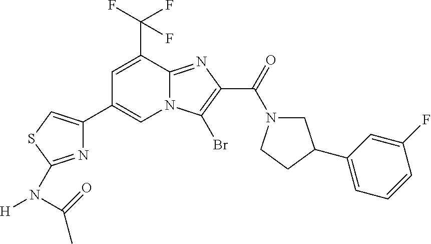 | N-(4-{3-Chloro-2-[3-(3-fluoro- phenyl)-pyrrolidine-1-carbonyl]- 8-trifluoromethyl-imidazo[1,2- a]pyridin-6-yl}-thiazol-2-yl)- acetamide |
| 645 | 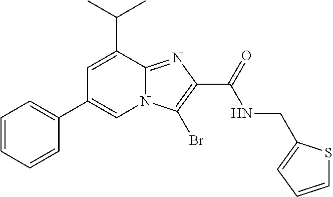 | 3-Bromo-8-isopropyl-6-phenyl- imidazo[1,2-a]pyridine-2- carboxylic acid (thiophen-2- ylmethyl)-amide |
| 646 | 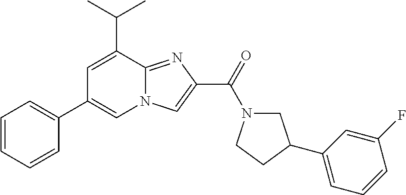 | [3-(3-Fluoro-phenyl)-pyrrolidin- 1-yl]-(8-isopropyl-6-phenyl- imidazo[1,2-a]pyridin-2-yl)- methanone |
| 647 | 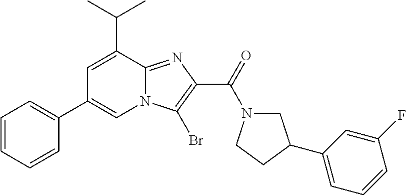 | (3-Bromo-8-isopropyl-6-phenyl- imidazo[1,2-a]pyridin-2-yl)-[3-(3- fluoro-phenyl)-pyrrolidin-1-yl]- methanone |
| 648 | 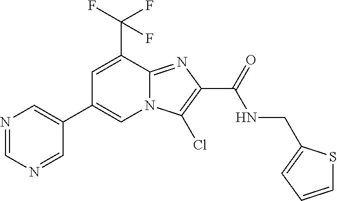 | 3-Chloro-6-pyrimidin-5-yl-8- trifluoromethyl-imidazo[1,2- a]pyridine-2-carboxylic acid (thiophen-2-ylmethyl)-amide |
| 649 | 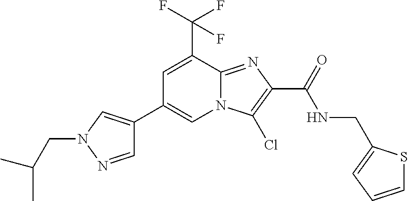 | 3-Chloro-6-(1-isobutyl-1H- pyrazol-4-yl)-8-trifluoromethyl- imidazo[1,2-a]pyridine-2- carboxylic acid (thiophen-2- ylmethyl)-amide |
| 650 | 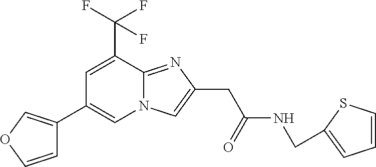 | 2-[6-(furan-3-yl)-8- (trifluoromethyl)imidazo[1,2- a]pyridin-2-yl]-N-(thiophen-2- ylmethyl)acetamide |
| 651 | 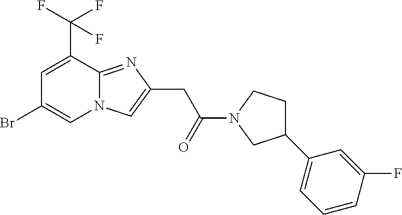 | 2-[6-bromo-8- (trifluoromethyl)imidazo[1,2- a]pyridin-2-yl]-1-[3-(3- fluorophenyl)pyrrolidin-1- yl]ethanone |
| 652 | 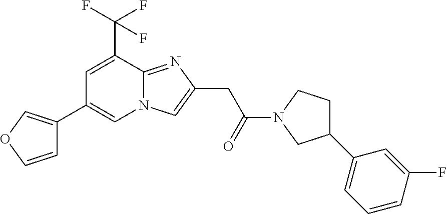 | 1-[3-(3-fluroophenyl)pyrrolidin-1- yl]-2-[6-(furan-3-yl)-8- (trifluoromethyl)imidazo[1,2- a]pyridin-2-yl]ethanone |
| 653 | 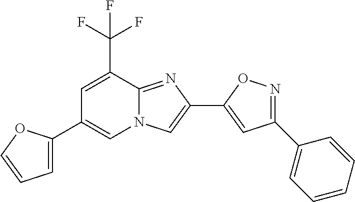 | 6-Furan-2-yl-2-(3-phenyl- isoxazol-5- yl)-8-trifluoromethyl- imidazo[1,2- a]pyridine |
| 654 | 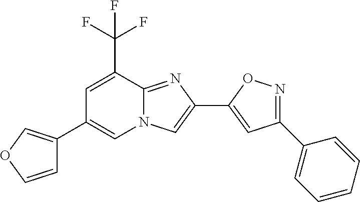 | 6-Furan-3-yl-2-(3-phenyl- isoxazol-5- yl)-8-trifluoromethyl- imidazo[1,2- a]pyridine |
| 655 | 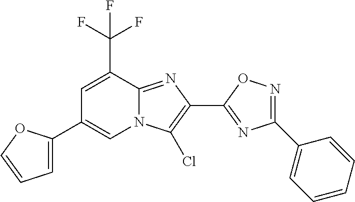 | 3-Chloro-6-furan-2-yl-2-(3- phenyl-[1,2,4] oxadiazol-5-yl)-8- trifluorometh- yl-imidazo[1,2-a]pyridine |
| 656 | 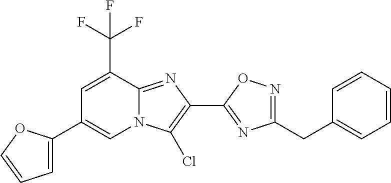 | 2-(3-Benzyl-[1,2,4]oxadiazol-5- yl)- 3-chloro-6-furan-2-yl-8- trifluorometh- yl-imidazo[1,2-a]pyridine |
| 657 | 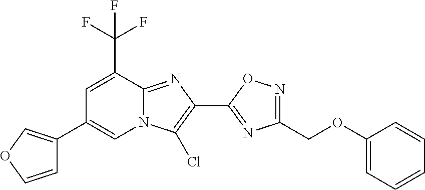 | 3-Chloro-6-furan-3-yl-2-(3- phenoxymeth- yl-[1,2,4]oxadiazol-5-yl)-8- trifluoromethyl-imidazo[1,2- a]pyridine |
| 658 | 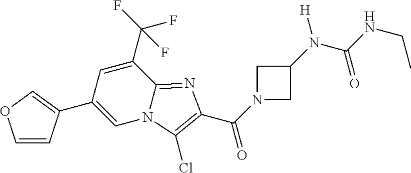 | 1-(1-{[3-chloro-6-(furan-3-yl)-8- (trifluoromethyl)imidazo[1,2- a]pyridin-2-yl]carbonyl}azetidin- 3-yl)-3-ethylurea |
| 659 | 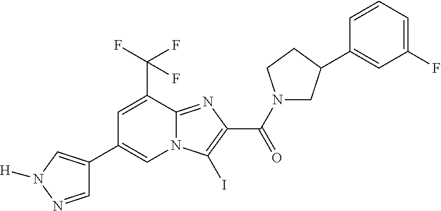 | [3-(3-fluroophenyl)pyrrolidin-1- yl][3-iodo-6-(1H-pyrazol-4-yl)-8- (trifluoromethyl)imidazo[1,2- a]pyridin-2-yl]methanone |

Also provided is a pharmaceutical composition comprising a pharmaceutically acceptable diluent and a therapeutically effective amount of at least one chemical entity described herein.

Also provided is a pharmaceutical composition comprising a pharmaceutically acceptable diluent and a therapeutically effective amount of at least one chemical entity chosen from compounds of Formula 1a

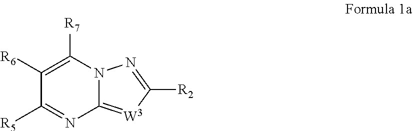

and pharmaceutically acceptable salts thereof, wherein

W³ is selected from CR³ and NR³;

R² is selected from halogen, optionally substituted alkyl, optionally substituted alkenyl, optionally substituted alkynyl, optionally substituted cycloalkyl, optionally substituted amino, optionally substituted heterocycloalkyl, optionally substituted aryl, optionally substituted heteroaryl, —OR¹⁵, —SR¹⁵, —S(O)R⁶, —S(O)₂R⁶, —S(O)₂NR¹⁰R¹¹, —NR¹⁰R¹¹, —NR¹¹C(O)NR¹⁰R¹¹, —NR¹¹C(S)NR¹⁰R¹¹, —NR¹¹S(O)₂R¹⁴—NR¹¹C(O)OR¹³, —NR¹¹C(O)R¹², —C(NR¹¹)NR¹⁰R¹¹, —C(O)NR¹⁰R¹¹, —C(O)OR¹³, —CN, —NO₂, and —C(O)R¹²;

R³ is absent or is selected from halogen, optionally substituted alkenyl, optionally substituted alkynyl, optionally substituted cycloalkyl, optionally substituted amino, optionally substituted heterocycloalkyl, optionally substituted aryl, optionally substituted heteroaryl, —OR¹⁵, —SR¹⁵, —S(O)R¹⁶, —S(O)₂R¹⁶, —S(O)₂NR¹⁰R¹¹, —NR¹⁰R¹¹, —NR¹¹C(O)NR¹⁰R¹¹—NR¹¹C(S)NR¹⁰R¹¹, —NR¹¹S(O)₂R¹⁴—NR¹¹C(O)OR¹³, —NR¹¹C(O)R¹², —C(NR¹¹)NR¹⁰R¹¹, —C(O)NR¹⁰R¹¹, —C(O)OR¹³, —CN, —NO₂, and —C(O)R¹²;

R⁵ is selected from halogen, optionally substituted alkyl, optionally substituted alkenyl, optionally substituted alkynyl, optionally substituted cycloalkyl, optionally substituted amino, optionally substituted heterocycloalkyl, optionally substituted aryl, optionally substituted heteroaryl, —OR¹⁵, —SR¹⁵, —S(O)R¹⁶, —S(O)₂R¹⁶, —S(O)₂NR¹⁰R¹¹, —NR¹⁰R¹¹, —NR¹¹C(O)NR¹⁰R¹¹, —NR¹¹C(S)NR¹⁰R¹¹, —NR¹¹S(O)₂R¹⁴—NR¹¹C(O)OR¹³, —NR¹¹C(O)R¹², —C(NR¹¹)NR¹⁰R¹¹, —C(O)NR¹⁰R¹¹, —C(O)OR³, —CN, —NO₂, and —C(O)R¹²;

R⁶ is selected from hydrogen, halogen, optionally substituted alkyl, optionally substituted alkenyl, optionally substituted alkynyl, optionally substituted cycloalkyl, optionally substituted amino, optionally substituted heterocycloalkyl, optionally substituted aryl, optionally substituted heteroaryl, —OR¹⁵, —SR¹⁵, —S(O)R¹⁶, —S(O)₂R¹⁶, —S(O)₂NR¹⁰R¹¹, —NR¹⁰R¹¹, —NR¹¹C(O)NR¹⁰R¹¹, —NR¹¹C(S)NR¹⁰R¹¹, —NR¹¹S(O)₂R¹⁴—NR¹¹C(O)OR¹³, —NR¹¹C(O)R¹², —C(NR¹¹)NR¹⁰R¹¹, —C(O)NR¹⁰R¹¹, —C(O)OR¹³, —CN, —NO₂, and —C(O)R¹²;

R⁷ is selected from halogen, optionally substituted alkyl, optionally substituted alkenyl, optionally substituted alkynyl, optionally substituted cycloalkyl, optionally substituted amino, optionally substituted heterocycloalkyl, optionally substituted aryl, optionally substituted heteroaryl, —OR¹⁵, —SR¹⁵, —S(O)R¹⁶, —S(O)₂R¹⁶, —S(O)₂NR¹⁰R¹¹, —NR¹⁰R¹¹, —NR¹¹C(O)NR¹⁰R¹¹, —NR¹¹C(S)NR¹⁰R¹¹, —NR¹¹S(O)₂R¹⁴—NR¹¹C(O)OR¹³, —NR¹¹C(O)R¹², —C(NR¹¹)NR¹⁰R¹¹, —C(O)NR¹⁰R¹¹, —C(O)OR¹³, —CN, —NO₂, and —C(O)R¹²;

R¹⁰ and R¹¹ are independently selected from hydrogen, optionally substituted alkyl, optionally substituted amino, optionally substituted alkoxy, optionally substituted cycloalkyl, optionally substituted heterocycloalkyl, optionally substituted aryl, and optionally substituted heteroaryl, or R¹⁰ and R¹¹, taken together with any intervening atoms, form a ring system selected from optionally substituted heterocycloalkyl, and optionally substituted heteroaryl;

R¹² is selected from hydrogen, optionally substituted alkyl, optionally substituted cycloalkyl, optionally substituted heterocycloalkyl, optionally substituted aryl, and optionally substituted heteroaryl;

R¹³ is selected from hydrogen, optionally substituted alkyl, optionally substituted cycloalkyl, optionally substituted heterocycloalkyl, optionally substituted aryl, and optionally substituted heteroaryl;

R¹⁴ is selected from optionally substituted alkyl, optionally substituted cycloalkyl, optionally substituted heterocycloalkyl, optionally substituted aryl, and optionally substituted heteroaryl;

R¹⁵ is selected from hydrogen, optionally substituted alkyl, optionally substituted cycloalkyl, optionally substituted heterocycloalkyl, optionally substituted aryl, and optionally substituted heteroaryl; and

R¹⁶ is selected from optionally substituted alkyl, optionally substituted cycloalkyl, optionally substituted heterocycloalkyl, optionally substituted aryl, and optionally substituted heteroaryl.

In some embodiments of compounds of Formula 1a, R² is selected from optionally substituted alkyl, —NR¹¹S(O)₂R¹⁴, —NR¹¹C(O)NR¹⁰R¹¹, —NR¹¹C(O)OR¹³—C(O)NR¹⁰R¹¹, and —C(O)OR¹³.

In some embodiments of compounds of Formula 1a, R² is —C(O)NR¹⁰R¹¹. In some embodiments of compounds of Formula 1a, R¹⁰ is selected from lower alkyl and hydrogen. In some embodiments of compounds of Formula 1a, R¹⁰ is selected from optionally substituted alkyl, optionally substituted cycloalkyl, optionally substituted heterocycloalkyl, and optionally substituted aryl.

In some embodiments of compounds of Formula 1a, R¹⁰ is —(CR¹⁷R¹⁸)ₙR¹⁹, wherein

R¹⁷ and R¹⁸ are independently selected from hydrogen, carboxy, optionally substituted aminocarbonyl, lower carboxy ester, and lower alkyl; n is 0, 1 or 2; and R¹⁹ is chosen from optionally substituted aryl and optionally substituted heteroaryl. In some embodiments of compounds of Formula 1a, R¹⁰ is benzyl, thiophen-2-yl-ethyl, thiophen-3-yl-methyl, furan-2-yl-methyl, and furan-3-yl-methyl, each of which is optionally substituted.

In some embodiments of compounds of Formula 1a, R¹⁰ and R¹¹, together with any intervening atoms, form an optionally substituted heterocycloalkyl. In some embodiments of compounds of Formula 1a, R¹⁰ and R¹¹, together with any intervening atoms, form a substituted 3- to 7-membered nitrogen containing heterocycloalkyl which optionally further includes one or two additional heteroatoms chosen from N, O, S and P(O), wherein said 3- to 7-membered nitrogen containing heterocycloalkyl is substituted with a group —Y—R³⁰ and optionally substituted with a second group R³¹, wherein

Y is a bond or is selected from —NR¹⁰—, —NR¹¹SO₂—, —O—, —S—, —C(O)NR¹⁰—, and —S(O)₂R¹⁰—;

R³⁰ is selected from optionally substituted alkyl, optionally substituted cycloalkyl, optionally substituted heterocycloalkyl, optionally substituted aryl, and optionally substituted heteroaryl; and

R³¹ is selected from halogen, optionally substituted alkyl, optionally substituted alkenyl, optionally substituted alkynyl, optionally substituted cycloalkyl, optionally substituted heterocycloalkyl, optionally substituted aryl, optionally substituted heteroaryl, optionally substituted alkoxy, —OH, —SH, —NO₂, —NR¹⁰R¹¹, —C(O)NR¹⁰R¹¹, —C(O)OR³, —SO₂NR¹⁰R¹¹, —NR¹¹C(S)NR¹⁰R¹¹, —NR¹¹C(O)NR¹⁰R¹¹, —CN, —NR¹¹SO₂R¹⁴, and —NR¹¹CO₂R¹³.

In some embodiments of compounds of Formula 1a, R¹⁰ and R¹¹, together with any intervening atoms, form an optionally substituted heterocycloalkyl. In some embodiments of compounds of Formula 1a, R¹⁰ and R¹¹, together with any intervening atoms, form a substituted 3- to 7-membered nitrogen containing heterocycloalkyl which optionally further includes one or two additional heteroatoms chosen from N, O, S and P(O), wherein said 3- to 7-membered nitrogen containing heterocycloalkyl is substituted with a group —Y—R³⁰ and optionally substituted with a second group R³¹, wherein

Y is a bond or is selected from —O—, —S—, —C(O)NR¹⁰—, and —S(O)₂R¹⁰—;

R³⁰ is selected from optionally substituted cycloalkyl, optionally substituted heterocycloalkyl, optionally substituted aryl, and optionally substituted heteroaryl; and

R³¹ is selected from halogen, optionally substituted alkyl, optionally substituted alkenyl, optionally substituted alkynyl, optionally substituted cycloalkyl, optionally substituted heterocycloalkyl, optionally substituted aryl, optionally substituted heteroaryl, optionally substituted alkoxy, —NO₂, —NR¹⁰R¹¹, —C(O)NR¹⁰R¹¹, —C(O)OR¹³, —SO₂NR¹⁰R¹¹, —NR¹¹C(S)NR¹⁰R¹¹, —NR¹¹C(O)NR¹⁰R¹¹, —CN, —NR¹¹SO₂R¹⁴, and —NR¹¹CO₂R¹³.

In some embodiments of compounds of Formula 1a, Y is a bond or is chosen from —NR¹⁰— and —O—. In some embodiments of compounds of Formula 1a, Y is a bond or is —O—. In some embodiments of compounds of Formula 1a, Y is a bond.

In some embodiments of compounds of Formula 1a, R³⁰ is selected from optionally substituted aryl and optionally substituted heteroaryl. In some embodiments of compounds of Formula 1a, R³⁰ is selected from phenyl, thiophen-2-yl, thiophen-3-yl, furan-2-yl, furan-3-yl, thiazol-2-yl, thiazol-4-yl, thiazol-5-yl, pyrazol-4-yl, imidazol-4-yl, and imidazol-2-yl. In some embodiments of compounds of Formula 1a, R³⁰ is selected from phenyl, thiophen-2-yl, thiophen-3-yl, furan-2-yl, and furan-3-yl of compounds of Formula 1a. In some embodiments, R³⁰ is phenyl. In some embodiments of compounds of Formula 1a, R³⁰ is optionally substituted alkyl. In some embodiments of compounds of Formula 1a, R³⁰ is optionally substituted lower alkyl. In some embodiments of compounds of Formula 1a, R³⁰ is lower alkyl. In some embodiments of compounds of Formula 1a, R³⁰ is methyl.

In some embodiments of compounds of Formula 1a, R² is optionally substituted heteroaryl. In some embodiments, R² is isoxazol-5-yl or [1,2,4]oxadiazol-5-yl, each of which is optionally substituted. In some embodiments of compounds of Formula 1a, R² is isoxazol-5-yl or [1,2,4]oxadiazol-5-yl, each of which is optionally substituted with a group chosen from optionally substituted aryl and optionally substituted alkyl. In some embodiments of compounds of Formula 1a, R² is isoxazol-5-yl or [1,2,4]oxadiazol-5-yl, each of which is optionally substituted with a group chosen from optionally substituted phenyl, optionally substituted benzyl, and optionally substituted phenoxymethyl. In some embodiments of compounds of Formula 1a, R² is isoxazol-5-yl or [1,2,4]oxadiazol-5-yl, each of which is optionally substituted with a group chosen from phenyl, benzyl, and phenoxymethyl.

In some embodiments of compounds of Formula 1a, R³ is halogen. In some embodiments of compounds of Formula 1a, R³ is selected from chlorine and bromine. In some embodiments of compounds of Formula 1a, R³ is chlorine.

In some embodiments of compounds of Formula 1a, R⁵ is selected from optionally substituted cycloalkyl, optionally substituted aryl, optionally substituted heteroaryl, and optionally substituted heterocycloalkyl. In some embodiments of compounds of Formula 1a, R⁵ is selected from optionally substituted cycloalkyl, optionally substituted aryl, and optionally substituted heteroaryl. In some embodiments of compounds of Formula 1a, R⁵ is selected from optionally substituted aryl and optionally substituted heteroaryl. In some embodiments of compounds of Formula 1a, R⁵ is selected from pyrid-3-yl, pyrazol-4-yl, phenyl, furan-2-yl, furan-3-yl, thiophen-2-yl, and thiophen-3-yl, each of which is optionally substituted. In some embodiments of compounds of Formula 1a, R⁵ is selected from phenyl, furan-2-yl, furan-3-yl, thiophen-2-yl, and thiophen-3-yl, each of which is optionally substituted. In some embodiments of compounds of Formula 1a, R⁵ is selected from phenyl, furan-2-yl, furan-3-yl, thiophen-2-yl, and thiophen-3-yl, each of which is optionally substituted with one or two groups chosen from lower alkyl, halogen, morpholinyl, trifluoromethyl, and lower alkoxy. In some embodiments of compounds of Formula 1a, R⁵ is selected from phenyl, 3-fluorophenyl, furan-2-yl, furan-3-yl, thiophen-2-yl, and thiophen-3-yl.

In some embodiments of compounds of Formula 1a, R⁶ is selected from hydrogen, halogen, optionally substituted alkyl, —OR¹⁵, —S(O)NR¹⁰R¹¹, —C(O)R¹², —NO₂, —C(O)NR¹⁰R¹¹, and —NR¹⁰R¹¹. In some embodiments of compounds of Formula 1a, R⁶ is selected from hydrogen, halogen, optionally substituted alkyl, —S(O)NR¹⁰R¹¹, —C(O)R¹², —NO₂, —C(O)NR¹⁰R¹¹, and —NR¹⁰R¹¹.

In some embodiments of compounds of Formula 1a, R¹¹ is hydrogen. In some embodiments of compounds of Formula 1a, R¹⁰ is selected from optionally substituted alkyl and optionally substituted cycloalkyl.

In some embodiments of compounds of Formula 1a, R¹⁰ and R¹¹, taken together with any intervening atoms, form an optionally substituted heterocycloalkyl ring.

In some embodiments of compounds of Formula 1a, R⁶ is selected from hydrogen, halogen, and optionally substituted alkyl. In some embodiments of compounds of Formula 1a, R⁶ is selected from hydrogen and halogen. In some embodiments of compounds of Formula 1a, R⁶ is hydrogen.

In some embodiments of compounds of Formula 1a, R⁷ is selected from halogen, optionally substituted alkyl, optionally substituted cycloalkyl, optionally substituted alkoxy, heterocycloalkyl, optionally substituted aryl, —SO₂NR¹⁰R¹¹, and —NR¹⁰R¹¹. In some embodiments of compounds of Formula 1a, R⁷ is selected from halogen, optionally substituted alkyl, optionally substituted cycloalkyl, optionally substituted alkoxy, heterocycloalkyl, optionally substituted aryl, and —NR¹⁰R¹¹. In some embodiments of compounds of Formula 1a, R⁷ is selected from optionally substituted alkyl, optionally substituted cycloalkyl, optionally substituted alkoxy, and —NR¹⁰R¹¹. In some embodiments of compounds of Formula 1a, R⁷ is selected from optionally substituted alkyl, optionally substituted alkoxy, and —NR¹⁰R¹¹. In some embodiments of compounds of Formula 1a, R⁷ is selected from optionally substituted lower alkoxy and optionally substituted lower alkyl.

In some embodiments of compounds of Formula 1a, R⁷ is polyhalogenated lower alkoxy. In some embodiments of compounds of Formula 1a, R⁷ selected from trifluoromethoxy and difluorochloromethoxy.

In some embodiments of compounds of Formula 1a, R⁷ is polyhalogenated lower alkyl. In some embodiments of compounds of Formula 1a, R⁷ is polyhalogenated methyl. In some embodiments of compounds of Formula 1a, R⁷ is selected from trifluoromethyl and difluorochloromethyl. In some embodiments of compounds of Formula 1a, R⁷ is trifluoromethyl.

In some embodiments of compounds of Formula 1a, R⁷ is —NR¹⁰R¹¹. In some embodiments of compounds of Formula 1a, R¹⁰ is hydrogen. In some embodiments of compounds of Formula 1a, R¹⁰ is optionally substituted lower alkyl. In some embodiments of compounds of Formula 1a, R¹⁰ is methyl. In some embodiments of compounds of Formula 1a, R¹⁰ is 2-hydroxyethyl.

Also provided is a pharmaceutical composition comprising a pharmaceutically acceptable diluent and a therapeutically effective amount of at least one chemical entity chosen from
(5-(5-chlorothiophen-2-yl)-7-(trifluoromethyl)pyrazolo[1,5-a]pyridin-2-yl)(3-(3,4-dimethoxyphenyl)-5-(2-hydroxyphenyl)-4,5-dihydro-1H-pyrazol-1-yl)methanone;
(5-(5-chlorothiophen-2-yl)-7-(trifluoromethyl)pyrazolo[1,5-a]pyridin-2-yl)(3-(2,5-dimethylphenyl)-5-(2-hydroxyphenyl)-4,5-dihydro-1H-pyrazol-1-yl)methanone; and
(5-(5-chlorothiophen-2-yl)-7-(trifluoromethyl)pyrazolo[1,5-a]pyridin-2-yl)(3-(3,4-dichlorophenyl)-5-(2-hydroxyphenyl)-4,5-dihydro-1H-pyrazol-1-yl)methanone,
   and pharmaceutically acceptable salts thereof.

The methods of synthesis for the provided chemical entities employ readily available starting materials using the following general methods and procedures. It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvent used, but such conditions can be determined by one skilled in the art by routine optimization procedures.

Additionally, the methods of this specification employ protecting groups which are necessary to prevent certain functional groups from undergoing undesired reactions. Suitable protecting groups for various functional groups as well as suitable conditions for protecting and deprotecting particular functional groups are well known in the art. For example, numerous protecting groups are described in T. W. Greene and G. M. Wuts, Protecting Groups in Organic Synthesis, Third Edition, Wiley, New York, 1999, and references cited therein.

Furthermore, the provided chemical entities may contain one or more chiral centers and such compounds can be prepared or isolated as pure stereoisomers, i.e., as individual enantiomers or diastereomers, or as stereoisomer-enriched mixtures. All such stereoisomers (and enriched mixtures) are included within the scope of this specification, unless otherwise indicated. Pure stereoisomers (or enriched mixtures) may be prepared using, for example, optically active starting materials or stereoselective reagents well-known in the art. Alternatively, racemic mixtures of such compounds can be separated using, for example, chiral column chromatography, chiral resolving agents and the like.

The starting materials for the following reactions are generally known compounds or can be prepared by known procedures or obvious modifications thereof. For example, many of the starting materials are available from commercial suppliers such as Aldrich Chemical Co. (Milwaukee, Wis., USA), Bachem (Torrance, Calif., USA), Ernka-Chemce or Sigma (St. Louis, Mo., USA). Others may be prepared by procedures, or obvious modifications thereof, described in standard reference texts such as Fieser and Fieser's Reagents for Organic Synthesis, Volumes 1-15 (John Wiley and Sons, 1991), Rodd's Chemistry of Carbon Compounds, Volumes 1-5 and Supplementals (Elsevier Science Publishers, 1989), Organic Reactions, Volumes 1-40 (John Wiley and Sons, 1991), March's Advanced Organic Chemistry, (John Wiley and Sons, 4th Edition), and Larock's Comprehensive Organic Transformations (VCH Publishers Inc., 1989). The synthesis of the compounds provided generally follows either a convergent or linear synthetic pathway as described below.

Unless specified to the contrary, the reactions described herein take place at atmospheric pressure, generally within a temperature range from −10° C. to 200° C. Further, except as employed in the Examples or as otherwise specified, reaction times and conditions are intended to be approximate, e.g., taking place at about atmospheric pressure within a temperature range of about −10° C. to about 110° C. over a period of about 1 to about 24 hours; reactions left to run overnight average a period of about 16 hours.

The terms “solvent,” “organic solvent,” and “inert solvent” each mean a solvent inert under the conditions of the reaction being described in conjunction therewith [including, for example, benzene, toluene, acetonitrile, tetrahydrofuran (“THF”), dimethylformamide (“DMF”), chloroform, methylene chloride (or dichloromethane), diethyl ether, methanol, N-methylpyrrolidone (“NMP”), pyridine and the like]. Unless specified to the contrary, the solvents used in the reactions described herein are inert organic solvents. Unless specified to the contrary, for each gram of the limiting reagent, one cc (or mL) of solvent constitutes a volume equivalent

Isolation and purification of the chemical entities and intermediates described herein can be effected, if desired, by any suitable separation or purification procedure such as, for example, filtration, extraction, crystallization, column chromatography, thin-layer chromatography or thick-layer chromatography, or a combination of these procedures. Specific illustrations of suitable separation and isolation procedures can be had by reference to the examples herein below. However, other equivalent separation or isolation procedures can also be used.

When desired, the (R)- and (S)-isomers may be resolved by methods known to those skilled in the art, for example by formation of diastereoisomeric salts or complexes which may be separated, for example, by crystallization; via formation of diastereoisomeric derivatives which may be separated, for example, by crystallization, gas-liquid or liquid chromatography; selective reaction of one enantiomer with an enantiomer-specific reagent, for example enzymatic oxidation or reduction, followed by separation of the modified and unmodified enantiomers; or gas-liquid or liquid chromatography in a chiral environment, for example on a chiral support, such as silica with a bound chiral ligand or in the presence of a chiral solvent. Alternatively, a specific enantiomer may be synthesized by asymmetric synthesis using optically active reagents, substrates, catalysts or solvents, or by converting one enantiomer to the other by asymmetric transformation.

Scheme 1 shows a method of assembling the imidazopyridine scaffold with various substituents. 2-Amino pyridine substituted with R⁷ is brominated by treatment with NBS in a solvent such as DMF. Substituted 2-aminopyridine 1.2 is cyclized to the imidazopyridine 1.3 by heating it with ethyl bromopyruvate in a solvent like DMF. Treatment of intermediate 1.3 with NCS in DMF affords the 3-chlorosubstituted imidazopyridine 1.4. Palladium mediated coupling reactions such as Suzuki couplings, Sonogashira couplings and Heck couplings can afford diversity at R⁵ in intermediates 1.5. Hydrolysis of the ester is effected by refluxing in 4N HCl and acetonitrile as co-solvent. The acid 1.6 is converted to amides 1.7 through standard amide coupling agents such as HBTU.

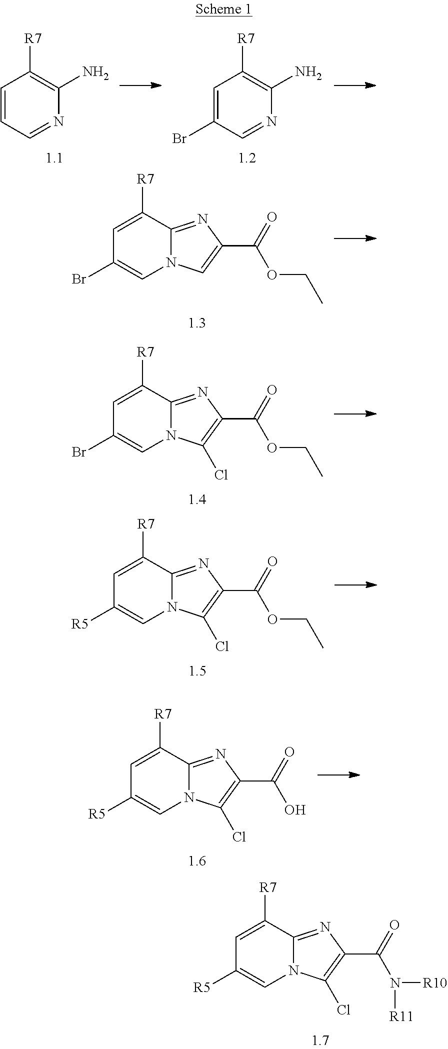

Scheme 2 shows a general scheme for the synthesis of purine analogs such as 2.5. An appropriately substituted amino dichloropyrimidine (2.1) can be converted to diaminopyrimidine such as 2.2 by stirring with an appropriately substituted primary amine (R³NH₂). Reaction with ethyl glyoxalate affords the ester intermediate 2.3. Paladium mediated coupling reactions such as Suzuki couplings, Sonogashira couplings and Heck couplings can afford diversity. Hydrolysis of the ester followed by amide coupling can afford the desired purine amide analogs such as 2.5.

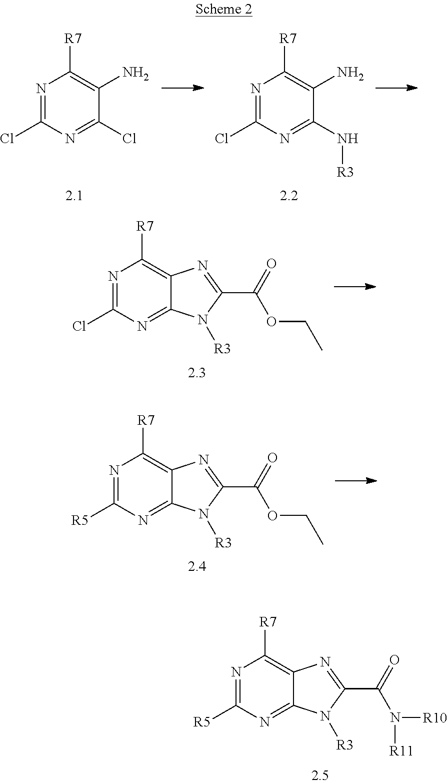

Scheme 3 shows a general scheme for the synthesis of pyrrolopyrimidines such as 3.7. The BOC protected amino bromo pyrimidine (3.2) can be prepared from the appropriately substituted amino bromo pyrimidine (3.1) using standard methods. Sonogashira coupling with ethyl propiolate would afford the alkyne 3.3. Cyclization to the 2-substituted pyrrolopyrimidine 3.4 can be done by heating with tetrabutyl ammonium fluoride. Heating 3.4 with an alkyl halide results in N-alkylation to the intermediate 3.5. Palladium mediated coupling reactions such as Suzuki couplings, Sonogashira couplings and Heck couplings can afford diversity at R⁵ in intermediates 3.6. Hydrolysis of the ester is effected by refluxing in 4N HCl and acetonitrile as co-solvent. The resulting acid is converted to amides 3.7 through standard amide coupling

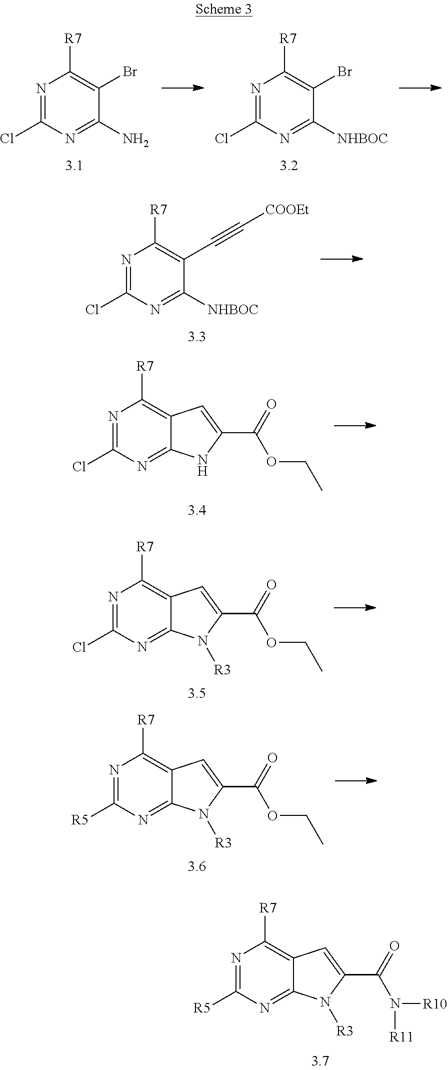

Scheme 4 describes the synthesis of imidazopyridine analogs such as 4.5. The appropriately substituted 3-amino 2-chloropyridine 4.1 when heated with a primary amine such as R3NH₂ affords the 2,3-diaminopyridine 4.2. Reaction with ethyl glyoxalate affords the ester intermediate 4.3. Hydrolysis of the ester followed by amide coupling can afford the desired imidazopyridine amide analogs such as 4.5.

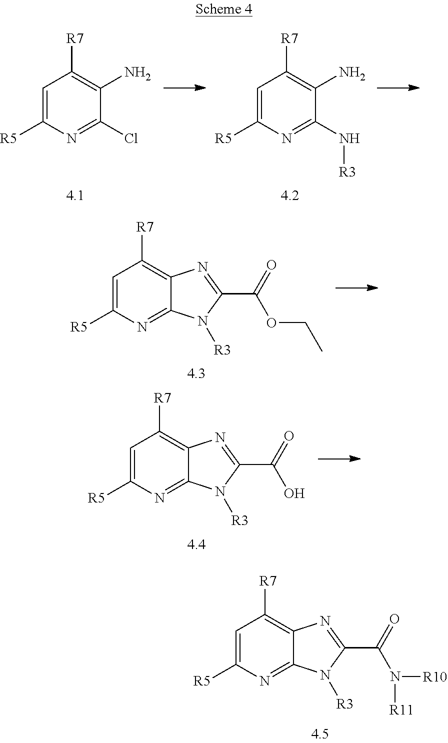

Scheme 5 describes the synthesis of pyrrolopyridine analogs such as 5.5. The appropriately substituted 3-aminopyridine such as 5.1 can be brominated at the 2-position by reaction with NBS. Sonogashira coupling with ethyl propiolate would afford the alkyne 5.3. Cyclization to the 2-substituted pyrolopyridine can be done by first protecting the amine as the Boc derivative, then heating with tetrabutyl ammonium fluoride. Hydrolysis of the ester is effected by refluxing in 4N HCl and acetonitrile as co-solvent. The resulting acid (5.4) is converted to amides 5.5 through standard amide coupling agents such as HBTU.

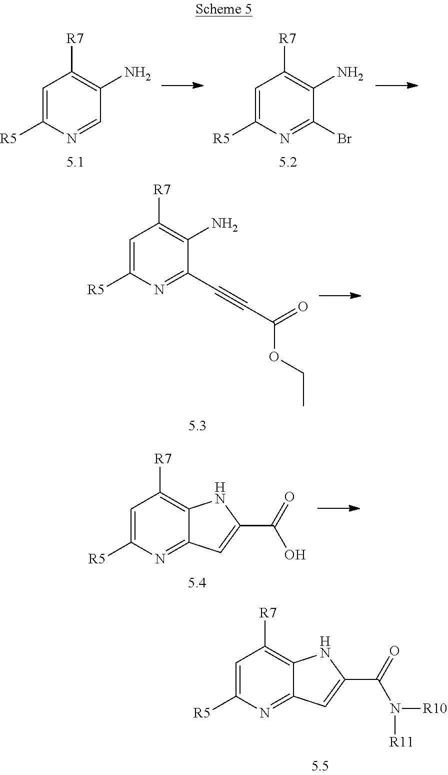

Scheme 6 shows the synthesis of pyrazolo[1,5-a]pyridines. Compounds can be prepared by 1,3-dipolar cycloaddition of substituted N-aminopyridines 6.2 with an alkyne such as methyl propiolate, dimethyl acetylenedicarboxylate or the like. N-amination of pyridines can be carried out by treating substituted pyridines 6.1 with aminating reagents such as hydroxylamine-O-sulfonic acid, O-mesitylenesulfonylhydroxylamine (MSH), O-(2,4-dinitrophenyl)hydroxylamine (Ref: C. Legault, A. B. Charette, J. Org. Chem., 2003, 68, 7119-7122; S. Lober, H. Htibner, W. Utz, P. Gmeiner, J. Med. Chem., 2001, 44, 2691-2694; also WO2006068826). Substituted pyridines can in turn be prepared by a variety of methods known in the literature such as the Chichibabin pyridine synthesis, Hantzsch pyridine synthesis, Guareschi-Thorpe pyridine synthesis, Bohlmann-Rahtz pyridine synthesis, Krohnke pyridine synthesis or Boger pyridine synthesis. Regarding the preparation of pyridines, see Comprehensive Heterocyclic Chemistry II Vol. 5, A. Katrizky, C. Rees, E. Scriven. For example, compounds of formula 6.3, can be prepared in which dimethyl acetylenedicarboxylate is treated with optionally substituted N-aminopyridine in the presence of a suitable base such as potassium carbonate, DBU and the like, in a suitable solvent such as DMF, and the like. Compounds of formula 6.4 can be prepared by the acidic hydrolysis and chemoselective decarboxylation with a suitable acid such as concentrated sulfuric acid and the like under heating conditions.

For example, compounds of formula 6.5, in which R² is C(O)NR¹⁰R¹¹ can be prepared by reacting a deprotected carboxylic acid with a primary or secondary amine or amine salt, e.g. amine of the formula NR¹⁰R¹¹.

The reaction can be carried out with the acid in the presence of a coupling agent such as benzotriazole-1-yloxytrispyrrolidino-phosphonium hexafluorophosphate (PyBOP®), bromo-tris-pyrrolidino-phosphonium hexafluorophosphate (PyBroP®), 2-(1H-benzotriazole-1-yl)-1,1,3,3-tetramethylaminium hexafluorophosphate (HBTU), O-(7-azabenzotriazol-1-yl)-N,N,N′,N′-tetramethyluronium hexafluorophosphate (HATU), or 1,3-dicyclohexylcarbodiimide (DCC) or 1-(3-dimethylaminopropyl)-3-ethylcarbodiimide hydrochloride (EDC) optionally in the presence of 1-hydroxybenzotriazole (HOBt). As appropriate, a base such as N,N-diisopropylethylamine, triethylamine, or N-methylmorpholine can be used. The reaction is carried out in suitable organic solvents, such as DMF, THF and the like. Suitable amines and amine salts are either commercially available or they can be prepared from commercial available starting materials by methods known in the art.

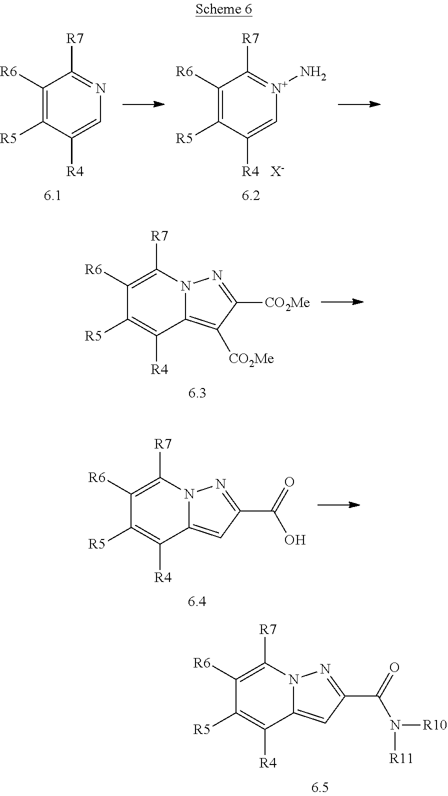

A compound of formula 7.4 or 7.5 in which R⁷ is Br, I, or alkyl can be prepared by deprotection of compound of formula 7.1 in which R⁷ is H with a base followed by addition of an electrophilic agent as shown in Scheme 7. This reaction is carried out in suitable organic solvents such as THF, ether and the like and at temperature about −78° C. Base such as n-buthyl lithium can be used for the deprotonation. Electrophilic reagents such as bromine, iodine, 1,2-dibromo-tetrachloroethane, methyl iodide can be used.

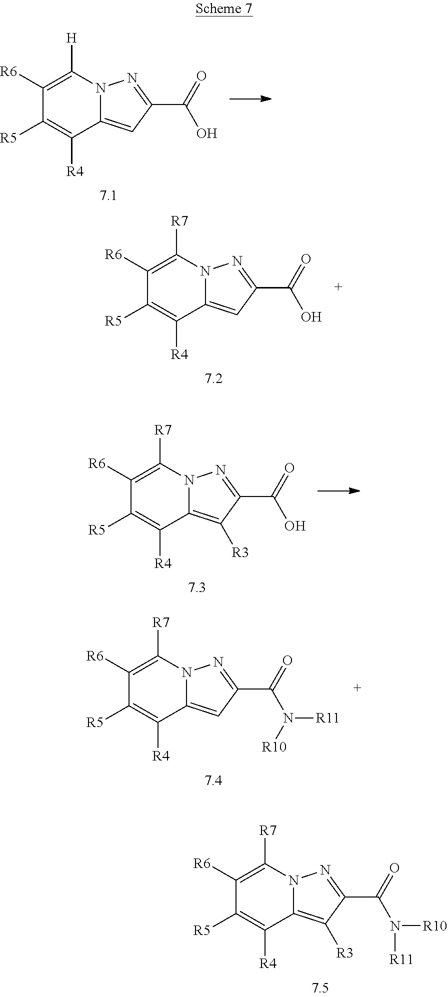

Referring to Scheme 8, a compound of formula 8.3 in which R³ is Cl, Br, or I, can be prepared by treating compounds of formula 8.1 or 8.4 in which R³ is H with electrophilic agents such as N-bromosuccinimide (NBS), N-chlorosuccinimide (NCS), N-iodosuccinimide (NIS). The reaction can be carried out in suitable solvents such as DMF, acetonitrile, chloroform, acetic acid and the like and at room temperature or heating at 40-50° C.

A compound of formula 8.3 in which R³ is NO₂ can be prepared by treating compounds of formula 8.1 in which R³ is H with nitrating agents such as fuming nitric acid, potassium nitrate or the like. The reaction can be carried out with suitable solvents such as sulfuric acid, acetic anhydride, trifluoroacetic acid and the like.

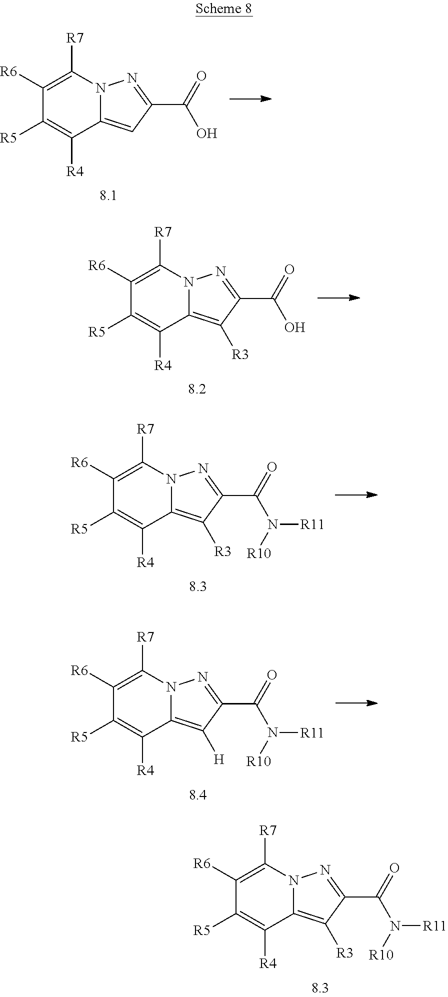

Referring to Scheme 9, a compound of formula 9.2 with R⁷ is NR¹⁰R¹¹ or OR¹⁵ can be prepared by substitution of a compound 9.1 with R⁷ is Br or Cl with an amine or alcohol in a suitable solvent such as DMF, DMA, NMP and the like. These reactions can be carried out at 120-200° C. under conventional heating or under microwave conditions.

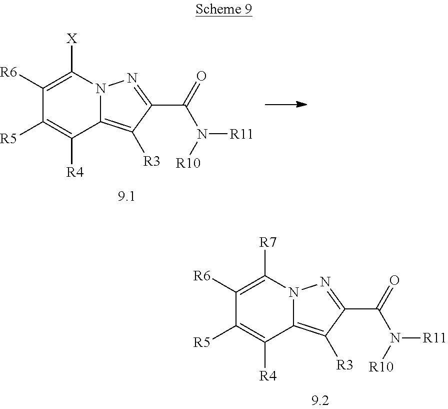

Referring to Scheme 10, a compound of formula 10.2 with R⁷ is CN, optionally substituted aryl, optionally substituted heteroaryl, or optionally substituted amino can be prepared by transition metal-mediated reactions of a compound with formula 10.1 with R⁷ is Cl, Br, or I. For example, these transition metal-mediated reactions can be one of those in the literature such as Suzuki-Miyaura reacions, Heck reactions, Stille reactions, Sonogashira reactions, and Buchwald aminations.

Similarly, a compound of formula 10.4 with R⁵ is CN, optionally substituted aryl, optionally substituted heteroaromatic rings, or optionally substituted amino can be prepared by transition metal-mediated reactions of a compound with formula 10.3 with R⁵ is Cl, Br, or I. For example, these transition metal-mediated reactions can be one of those in the literature such as Suzuki-Miyaura reacions, Heck reactions, Stille reactions, Sonogashira reactions, and Buchwal-Hartwig aminations.

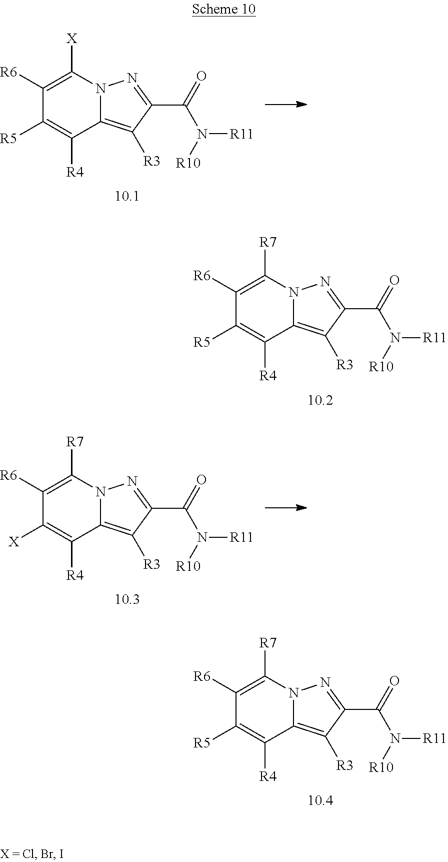

Referring to Scheme 11, compounds of formula 11.10 in which R⁷ is polyhalogenated alkyl, such as CF₂C1 or CF₃, can be prepared. Pyrazolo[1,5-a]pyridines may be prepared by Hemetsberger-Knittel synthesis by thermolysis of substituted 2-azido-2-pyridine acrylate of formula 11.8. (K. L. Stevens, et al, Org. Lett., 2005, 7, 4653-4756; P. J. Roy, et al., Synthesis, 2005, 16, 2751-2757.)

Substituted pyridines of formula 11.5 with R⁷ is polyhalogenated alkyl, such as CF₃, or CF₂Cl, can be prepared using the Krohnke pyridine synthesis (F. Krihnke, Synthesis, 1976, 1-24) by reacting a pyridinium salt of formula 11.4 and 4-substituted-2-oxo-but-3-enoic acid or its acid salt in the presence of ammonium acetate. The reaction can be carried out in suitable solvents such as methanol, acetic acid, water and the like and heating at 80-100° C. maybe used.

Pyridinium salt of formula 11.4 in which R⁷ is CF₂C₁ or CF₃ can be prepared by reacting 1-carboxymethylpyridinium chloride 11.1 (T. Thorsteinsson, et al, J. Med. Chem. 2003, 46, 4173-4181) with anhydrides such as trifluoroacetic anhydride, dichlorofluoroacetic anhydride in the presence of a base. As appropriate, a base such as N,N-diisopropylethylamine, or triethylamine can be used. The reaction is carried out in suitable organic solvents, such as ether, THF or the like and at temperature around 0° C. The betaeine of formula 11.3 can be hydrolyzed under acidic conditions to give Pyridinium salt of formula 11.4. Acids such as hydrochloric acid can be used and heating at 40-80° C. may be used.

Substituted-2-oxo-but-3-enoic acid can be obtained from commercial sources or can be prepared as known in the art. Compounds with R⁵ is furan-2-yl can be prepared by reacting 2-furaldehyde with pyruvic acid in the presence of base. Suitable bases such as aqueous sodium hydroxide or aqueous potassium hydroxide can be used and temperature around 0° C. may be used.

Substituted pyridine 2-carboxyaldehyde 11.6 can be prepared by conversion of pyridine 2-carboxylic acid 11.5 to an ester followed by reduction with hydride reagents such as lithium aluminum hydride (LAH), di-isobutylaluminum hydride (DIBAL-H) and the like. The reaction can be carried out in suitable solvents such Et₂O, THF and the like and temperatures of from about −78 to 0° C. may be used. Alternatively, substituted pyridine 2-carboxyaldehyde 11.6 can be prepared by conversion of pyridine 2-carboxylic acid 11.5 to a Weinreb amide followed by reduction with hydride reagents such as lithium aluminum hydride (LAH), di-isobutylaluminum hydride (DIBAL-H) and the like. The reaction can be carried out in suitable solvents such Et₂O, THF and the like and temperatures of from about −78 to 0° C. may be used.

Substituted pyridine 2-carboxyaldehyde 11.6 can react with an alkyl azido acetate 11.7 under basic condition to give substituted 2-azido-2-pyridine acrylate of formula 11.8. Suitable bases such as sodium methoxide, sodium ethoxide, sodium tert-butoxide and the like can be used. The reaction can be carried out in suitable solvents such as methanol, ethanol, iso-propanol, tert-butanol and the like and the temperatures of from about −50 to 0° C. may be used.

Pyrazolo[1,5-a]pyridines of formula 11.9 can be prepared by heating substituted 2-azido-2-pyridine acrylate of formula 11.8. The reaction can be carried out in suitable solvents such as toluene, xylene, DMF, DMA, NMP and the like. These reactions can be carried out at 120-200° C. under conventional heating or under microwave conditions.

Esters of pyrazolo[1,5-a]pyridines of formula 11.9 can be saponified under basic conditions such as lithium hydroxide, sodium hydroxide, potassium hydroxide and the like. The reaction can be carried out in suitable solvents such as THF, methanol and the like with the addition of water. These reactions can be carried out at room temperature or optionally with heating. Similarly, the acids obtained can be coupled with an amine NHR¹⁰R¹¹ or amine salt to give compounds of formula 11.10 under standard amide coupling conditions described above.

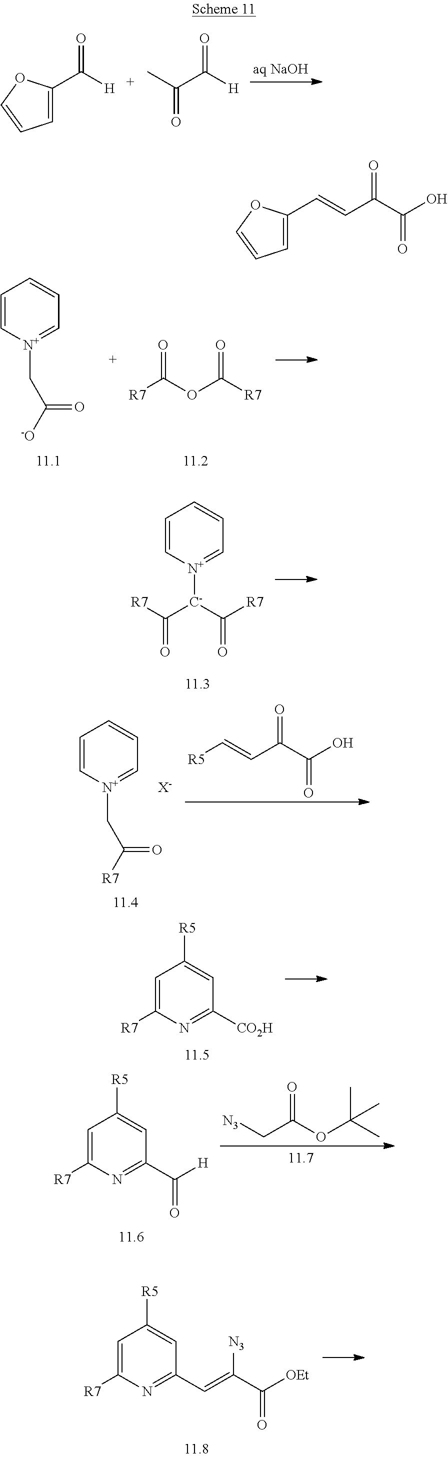

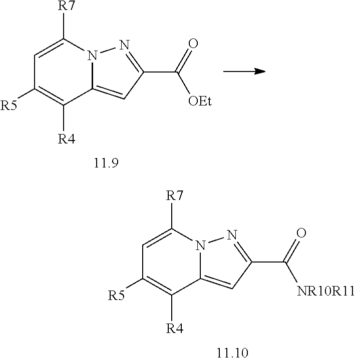

Scheme 12 describes the synthesis of imidazo[1,2-b]pyridazine analogs such as 12.6. The appropriately substituted 2-chloropyridazine 12.1 can be aminated with ammonia in solvents such as iso-propanol to give 2-aminopyridazine 12.2 and the reaction is usually carried out under heating in a sealed tube. 2-Chloropyridazine can in turn be prepared from chlorination of 2H-pyridazin-3-one with phosphoryl chloride and the like. Substituted 2-aminopyridazine can be cyclized with substituted methyl bromopyruvate in solvents such as DMF and the like and at temperatures 50-80° C. to give substituted imidazo[1,2-b]pyridazine 12.3. Halogenation at the 3-position can be carried out by reacting imidazo[1,2-b]pyridazine 12.3 with N-chlorosuccinimide, N-bromosuccinimide, N-iodosuccinimide and the like. The methyl ester of substituted imidazo[1,2-b]pyridazine 12.4 can be saponified with bases such as lithium hydroxide, sodium hydroxide, and the like and in solvents such as tetrahydrofuran, alcohol, and water. Substituted imidazo[1,2-b]pyridazine-2-carboxylic acids 12.5 can be converted to the amides 12.6 in the presence of a coupling agent such as benzotriazole-1-yloxytrispyrrolidino-phosphonium hexafluorophosphate (PyBOP®), bromo-tris-pyrrolidino-phosphonium hexafluorophosphate (PyBroP®), 2-(1H-benzotriazole-1-yl)-1,1,3,3-tetramethylaminium hexafluorophosphate (HBTU), O-(7-azabenzotriazol-1-yl)-N,N,N′,N′-tetramethyluronium hexafluorophosphate (HATU), or 1,3-dicyclohexylcarbodiimide (DCC) or 1-(3-dimethylaminopropyl)-3-ethylcarbodiimide hydrochloride (EDC) optionally in the presence of 1-hydroxybenzotriazole (HOBt). As appropriate, a base such as N,N-diisopropylethylamine, triethylamine, or N-methylmorpholine can be used. The reaction is carried out in suitable organic solvents, such as DMF, THF and the like. Suitable amines and amine salts are either commercially available or they can be prepared from commercial available starting materials by methods known in the art.

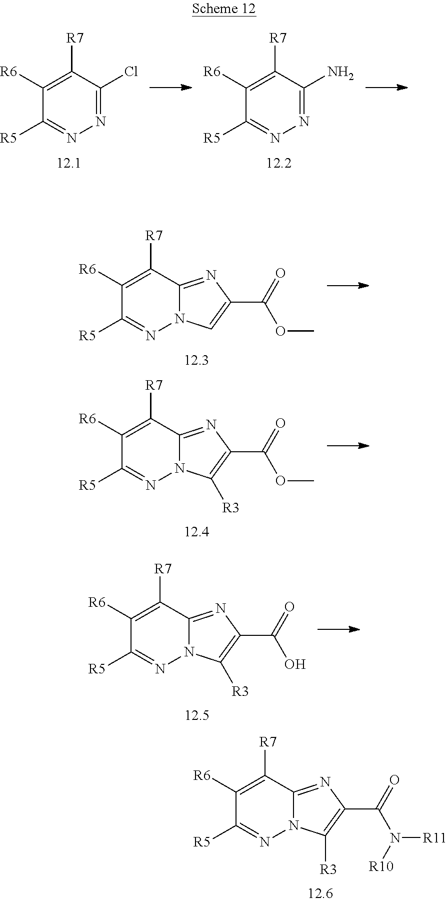

Scheme 13 describes the synthesis of benzimidazole analogs such as 13.7 and 13.8. Benzimidazole scaffold can be assembled by cyclization of substituted 2-acyl-1,2-diaminophenediamine. Substituted aniline 13.1 can be acylated with ethyl oxalyl chloride to give substituted N-phenyl-oxalamic acid ethyl ester 13.2 which in turn can be nitrated using nitric acid/sulfuric acid to give substituted N-(2-nitro-phenyl)-oxalamic acid ethyl ester 13.3. Reduction of nitro group can be carried out using sodium dithionite or other reducing reagents. Addition of aromatic or heteroaromatic groups with concomitant cyclization to benimidazole and saponification of ethyl ester can be achieved under Suzuki coupling conditions. The resultant substituted benzimidazole-2-carboxylic acids 13.5 can be converted to the amides 13.6 using standard coupling conditions as described above. Alkylation of benzimidazole can be carried out using alkyl halides, alkyl mesylate, alkyl triflates or the like and with suitable bases such as sodium hydride in solvents such as DMF, THF and the like, to give benzimidazole analogs 13.7 and 13.8

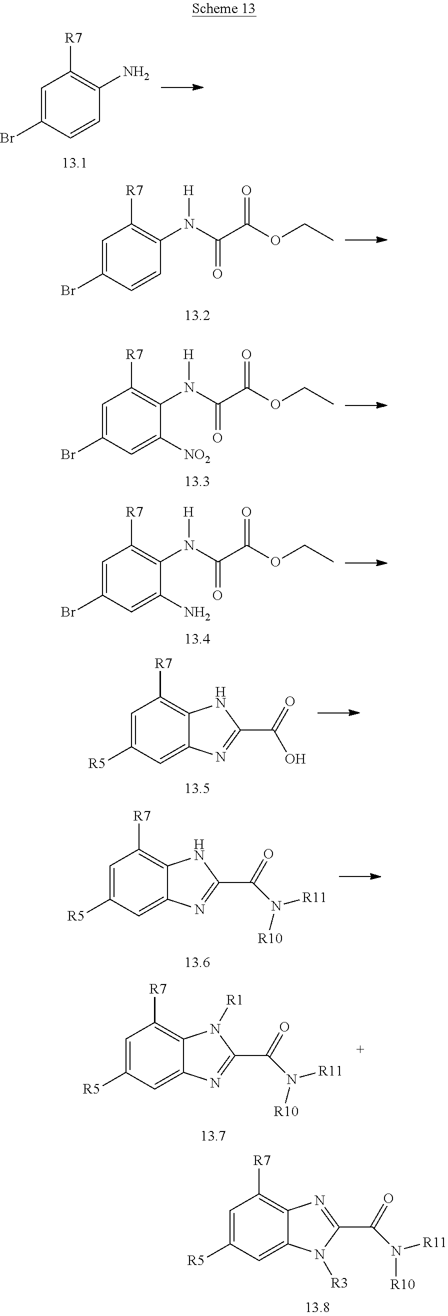

Alternatively, 1-alkyl-1H-benzimidazole derivatives can be prepared in Scheme 14. N-alkylation of substituted N-(2-nitro-phenyl)-oxalamic acid ethyl ester 14.1 can be prepared with alkyl halides, alkyl mesylates, alkyl triflates or the like with suitable bases such as sodium hydride in solvents such as DMF, THF and the like. Reduction of nitro group can be carried out using sodium dithionite or other reducing reagents. Addition of aromatic or heteroaromatic groups with concomitant cyclication to benzimidazole and saponification of ethyl ester can be achieved under Suzuki coupling conditions. The resultant substituted 1-alkyl-1H-benzoimidazole-2-carboxylic acids 14.4 can be converted to the amides 14.5 using standard coupling conditions as described above.

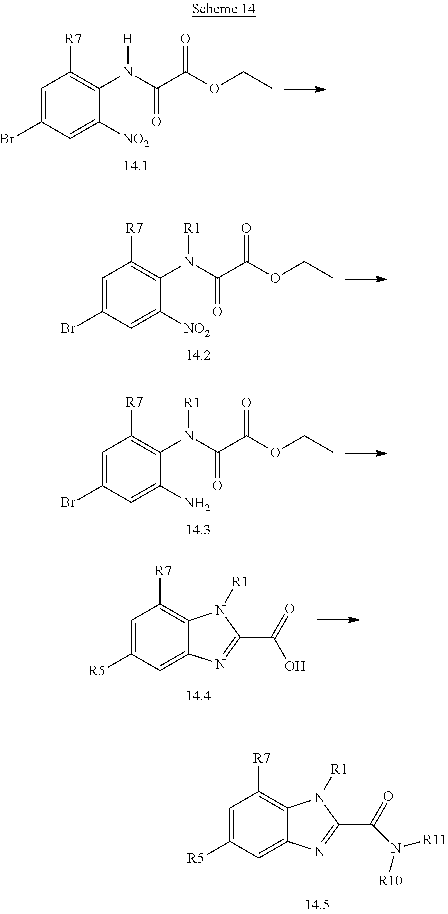

Provided are chemical entities possessing antiviral activity, including against hepatitis C virus. The chemical entities provided herein may inhibit viral replication by inhibiting the enzymes involved in replication, including RNA dependent RNA polymerase. They may also inhibit other enzymes utilized in the activity or proliferation of viruses in the flaviviridae family, such as HCV.

The chemical entities described herein are administered at a therapeutically effective dosage, e.g., a dosage sufficient to provide treatment for the disease states previously described. While human dosage levels have yet to be optimized for the chemical entities described herein, generally, a daily dose ranges from about 0.05 to 100 mg/kg of body weight; in certain embodiments, from about 0.10 to 10.0 mg/kg of body weight, and in certain embodiments, from about 0.15 to 1.0 mg/kg of body weight. Thus, for administration to a 70 kg person, in certain embodiments, the dosage range would be about from 3.5 to 7000 mg per day; in certain embodiments, about from 7.0 to 700.0 mg per day, and in certain embodiments, about from 10.0 to 100.0 mg per day. The amount of the chemical entity administered will, of course, be dependent on the subject and disease state being treated, the severity of the affliction, the manner and schedule of administration and the judgment of the prescribing physician; for example, a likely dose range for oral administration would be from about 70 to 700 mg per day, whereas for intravenous administration a likely dose range would be from about 70 to 700 mg per day depending on compound pharmacokinetics.

Administration of the chemical entities described herein can be via any of the accepted modes of administration for agents that serve similar utilities including, but not limited to, orally, sublingually, subcutaneously, intravenously, intranasally, topically, transdermally, intraperitoneally, intramuscularly, intrapulmonarilly, vaginally, rectally, or intraocularly. In some embodiments, oral or parenteral administration is used.

Pharmaceutical compositions or formulations include solid, semi-solid, liquid and aerosol dosage forms, such as, e.g., tablets, capsules, powders, liquids, suspensions, suppositories, aerosols or the like. The chemical entities can also be administered in sustained or controlled release dosage forms, including depot injections, osmotic pumps, pills, transdermal (including electrotransport) patches, and the like, for prolonged and/or timed, pulsed administration at a predetermined rate. In certain embodiments, the compositions are provided in unit dosage forms suitable for single administration of a precise dose.

The chemical entities described herein can be administered either alone or more typically in combination with a conventional pharmaceutical carrier, excipient or the like (e.g., mannitol, lactose, starch, magnesium stearate, sodium saccharine, talcum, cellulose, sodium crosscarmellose, glucose, gelatin, sucrose, magnesium carbonate, and the like). If desired, the pharmaceutical composition can also contain minor amounts of nontoxic auxiliary substances such as wetting agents, emulsifying agents, solubilizing agents, pH buffering agents and the like (e.g., sodium acetate, sodium citrate, cyclodextrine derivatives, sorbitan monolaurate, triethanolamine acetate, triethanolamine oleate, and the like). Generally, depending on the intended mode of administration, the pharmaceutical composition will contain about 0.005% to 95%; in certain embodiments, about 0.5% to 50% by weight of a chemical entity. Actual methods of preparing such dosage forms are known, or will be apparent, to those skilled in this art; for example, see Remington's Pharmaceutical Sciences, Mack Publishing Company, Easton, Pa.

In addition, the chemical entities described herein can be co-administered with, and the pharmaceutical compositions can include, other medicinal agents, pharmaceutical agents, adjuvants, and the like. Suitable medicinal and pharmaceutical agents include therapeutically effective amounts of one or more agents active against HCV. In some embodiments, the agent active against HCV is an inhibitor of HCV proteases, HCV polymerase, HCV helicase, HCV NS4B protein, HCV entry, HCV assembly, HCV egress, HCV replicase, HCV NS5A protein, or inosine 5′-monophosphate dehydrogenase. In some embodiments, the agent active against HCV is an inhibitor of HCV proteases, HCV polymerase, HCV helicase, HCV NS4B protein, HCV entry, HCV assembly, HCV egress, HCV NS5A protein, or inosine 5′-monophosphate dehydrogenase.

Active agents against HCV include ribavirin, levovirin, viramidine, thymosin alpha-1, an inhibitor of NS3 serine protease, and inhibitor of inosine monophosphate dehydrogenase, interferon-alpha, either alone or in combination with ribavirin or levovirin. In some embodiments, the additional agent active against HCV is interferon-alpha or pegylated interferon-alpha alone or in combination with ribavirin or levovirin. In some embodiments, the agent active against hepatitis C virus is interferon.

Other suitable medicinal and pharmaceutical agents include TRH, diethylstilbesterol, theophylline, enkephalins, E series prostaglandins, compounds disclosed in U.S. Pat. No. 3,239,345 (e.g., zeranol), compounds disclosed in U.S. Pat. No. 4,036,979 (e.g., sulbenox), peptides disclosed in U.S. Pat. No. 4,411,890 growth hormone secretagogues such as GHRP-6, GHRP-1 (disclosed in U.S. Pat. No. 4,411,890 and publications WO 89/07110 and WO 89/07111), GHRP-2 (disclosed in WO 93/04081), NN703 (Novo Nordisk), LY444711 (Lilly), MK-677 (Merck), CP424391 (Pfizer) and B-HT920, growth hormone releasing factor and its analogs, growth hormone and its analogs and somatomedins including IGF-1 and IGF-2, alpha-adrenergic agonists, such as clonidine or serotonin 5-HT_{D} agonists, such as sumatriptan, agents which inhibit somatostatin or its release, such as physostigmine, pyridostigmine, parathyroid hormone, PTH(1-34), and bisphosphonates, such as MK-217 (alendronate).

Still other suitable medicinal and pharmaceutical agents include estrogen, testosterone, selective estrogen receptor modulators, such as tamoxifen or raloxifene, other androgen receptor modulators, such as those disclosed in Edwards, J. P. et. al., Bio. Med. Chem. Let., 9, 1003-1008 (1999) and Hamann, L. G. et. al., J. Med. Chem., 42, 210-212 (1999), and progesterone receptor agonists (“PRA”), such as levonorgestrel, medroxyprogesterone acetate (MPA).

Still other suitable medicinal and pharmaceutical agents include HIV and AIDS therapies, such as indinavir sulfate, saquinavir, saquinavir mesylate, ritonavir, lamivudine, zidovudine, lamivudine/zidovudine combinations, zalcitabine, didanosine, stavudine, and megestrol acetate.

Still other suitable medicinal and pharmaceutical agents include antiresorptive agents, hormone replacement therapies, vitamin D analogues, elemental calcium and calcium supplements, cathepsin K inhibitors, MMP inhibitors, vitronectin receptor antagonists, Src SH.sub.2 antagonists, vacular—H⁺-ATPase inhibitors, ipriflavone, fluoride, Tibo lone, pro stanoids, 17-beta hydroxysteroid dehydrogenase inhibitors and Src kinase inhibitors.

The above other therapeutic agents, when employed in combination with the chemical entities described herein, may be used, for example, in those amounts indicated in the Physicians' Desk Reference (PDR) or as otherwise determined by one of ordinary skill in the art.

In certain embodiments, the compositions will take the form of a pill or tablet and thus the composition will contain, along with the active ingredient, a diluent such as lactose, sucrose, dicalcium phosphate, or the like; a lubricant such as magnesium stearate or the like; and a binder such as starch, gum acacia, polyvinylpyrrolidine, gelatin, cellulose, cellulose derivatives or the like. In another solid dosage form, a powder, marume, solution or suspension (e.g., in propylene carbonate, vegetable oils or triglycerides) is encapsulated in a gelatin capsule.

Liquid pharmaceutically administrable compositions can, for example, be prepared by dissolving, dispersing, etc. at least one chemical entity and optional pharmaceutical adjuvants in a carrier (e.g., water, saline, aqueous dextrose, glycerol, glycols, ethanol or the like) to form a solution or suspension. Injectables can be prepared in conventional forms, either as liquid solutions or suspensions, as emulsions, or in solid forms suitable for dissolution or suspension in liquid prior to injection. The percentage of chemical entities contained in such parenteral compositions is highly dependent on the specific nature thereof, as well as the activity of the chemical entities and the needs of the subject. However, percentages of active ingredient of 0.01% to 10% in solution are employable, and will be higher if the composition is a solid which will be subsequently diluted to the above percentages. In certain embodiments, the composition will comprise from about 0.2 to 2% of the active agent in solution.

Pharmaceutical compositions of the chemical entities described herein may also be administered to the respiratory tract as an aerosol or solution for a nebulizer, or as a microfine powder for insufflation, alone or in combination with an inert carrier such as lactose. In such a case, the particles of the pharmaceutical composition have diameters of less than 50 microns, in certain embodiments, less than 10 microns.

The following examples serve to more fully describe the manner of using the above-described invention. It is understood that these examples in no way serve to limit the true scope of this invention, but rather are presented for illustrative purposes.

In general, the chemical entities provided will be administered in a therapeutically effective amount by any of the accepted modes of administration for agents that serve similar utilities. The actual amount of the chemical entity, i.e., the active ingredient, will depend upon numerous factors such as the severity of the disease to be treated, the age and relative health of the subject, the potency of the chemical entity used, the route and form of administration, and other factors. The drug can be administered more than once a day, such as once or twice a day.

Therapeutically effective amounts of the chemical entities described herein may range from approximately 0.05 to 50 mg per kilogram body weight of the recipient per day; such as about 0.01-25 mg/kg/day, for example, from about 0.5 to 10 mg/kg/day. Thus, for administration to a 70 kg person, the dosage range may be about 35-70 mg per day.

In general, the chemical entities will be administered as pharmaceutical compositions by any one of the following routes: oral, systemic (e.g., transdermal, intranasal or by suppository), or parenteral (e.g., intramuscular, intravenous or subcutaneous) administration. In certain embodiments, oral administration with a convenient daily dosage regimen that can be adjusted according to the degree of affliction may be used. Compositions can take the form of tablets, pills, capsules, semisolids, powders, sustained release formulations, solutions, suspensions, elixirs, aerosols, or any other appropriate compositions. Another manner for administering the provided chemical entities is inhalation.

The choice of formulation depends on various factors such as the mode of drug administration and bioavailability of the drug substance. For delivery via inhalation the chemical entity can be formulated as liquid solution, suspensions, aerosol propellants or dry powder and loaded into a suitable dispenser for administration. There are several types of pharmaceutical inhalation devices-nebulizer inhalers, metered dose inhalers (MDI) and dry powder inhalers (DPI). Nebulizer devices produce a stream of high velocity air that causes the therapeutic agents (which are formulated in a liquid form) to spray as a mist that is carried into the patient's respiratory tract. MDI's typically are formulation packaged with a compressed gas. Upon actuation, the device discharges a measured amount of therapeutic agent by compressed gas, thus affording a reliable method of administering a set amount of agent. DPI dispenses therapeutic agents in the form of a free flowing powder that can be dispersed in the patient's inspiratory air-stream during breathing by the device. In order to achieve a free flowing powder, the therapeutic agent is formulated with an excipient such as lactose. A measured amount of the therapeutic agent is stored in a capsule form and is dispensed with each actuation.

Recently, pharmaceutical compositions have been developed for drugs that show poor bioavailability based upon the principle that bioavailability can be increased by increasing the surface area i.e., decreasing particle size. For example, U.S. Pat. No. 4,107,288 describes a pharmaceutical formulation having particles in the size range from 10 to 1,000 nM in which the active material is supported on a cross-linked matrix of macromolecules. U.S. Pat. No. 5,145,684 describes the production of a pharmaceutical formulation in which the drug substance is pulverized to nanoparticles (average particle size of 400 nm) in the presence of a surface modifier and then dispersed in a liquid medium to give a pharmaceutical formulation that exhibits remarkably high bioavailability.

The compositions are comprised of, in general, at least one chemical entity described herein in combination with at least one pharmaceutically acceptable excipient. Acceptable excipients are non-toxic, aid administration, and do not adversely affect the therapeutic benefit of the at least one chemical entity described herein. Such excipient may be any solid, liquid, semi-solid or, in the case of an aerosol composition, gaseous excipient that is generally available to one of skill in the art.

Solid pharmaceutical excipients include starch, cellulose, talc, glucose, lactose, sucrose, gelatin, malt, rice, flour, chalk, silica gel, magnesium stearate, sodium stearate, glycerol monostearate, sodium chloride, dried skim milk and the like. Liquid and semisolid excipients may be selected from glycerol, propylene glycol, water, ethanol and various oils, including those of petroleum, animal, vegetable or synthetic origin, e.g., peanut oil, soybean oil, mineral oil, sesame oil, etc. Liquid carriers, for injectable solutions, include water, saline, aqueous dextrose, and glycols.

Compressed gases may be used to disperse a chemical entity described herein in aerosol form. Inert gases suitable for this purpose are nitrogen, carbon dioxide, etc. Other suitable pharmaceutical excipients and their formulations are described in Remington's Pharmaceutical Sciences, edited by E. W. Martin (Mack Publishing Company, 18th ed., 1990).

The amount of the chemical entity in a composition can vary within the full range employed by those skilled in the art. Typically, the composition will contain, on a weight percent (wt %) basis, from about 0.01-99.99 wt % of at least one chemical entity described herein based on the total composition, with the balance being one or more suitable pharmaceutical excipients. In certain embodiments, the at least one chemical entity described herein is present at a level of about 1-80 wt %. Representative pharmaceutical compositions containing at least one chemical entity described herein are described below.

Additionally, the present specification is directed to a pharmaceutical composition comprising a therapeutically effective amount of at least one chemical entity described herein in combination with a therapeutically effective amount of another active agent against RNA-dependent RNA virus and, in particular, against HCV. Agents active against HCV include, but are not limited to, ribavirin, levovirin, viramidine, thymosin alpha-1, an inhibitor of HCV NS3 serine protease, or an inhibitor of inosine monophosphate dehydrognease, interferon-a, pegylated interferon-α (peginterferon-a), a combination of interferon-a and ribavirin, a combination of peginterferon-a and ribavirin, a combination of interferon-a and levovirin, and a combination of peginterferon-a and levovirin. Interferon-a includes, but is not limited to, recombinant interferon-a2a (such as ROFERON interferon available from Hoffman-LaRoche, Nutley, N.J.), interferon-a2b (such as Intron-A interferon available from Schering Corp., Kenilworth, N.J., USA), a consensus interferon, and a purified interferon-a product. For a discussion of ribavirin and its activity against HCV, see J. O, Saunders and S. A. Raybuck, “Inosine Monophosphate Dehydrogenase: Consideration of Structure, Kinetics and Therapeutic Potential,” Ann. Rep. Med. Chem., 2:201-210 (2000).

The following examples serve to more fully describe the manner of using the above-described invention. It is understood that these examples in no way serve to limit the true scope of the invention, but rather are presented for illustrative purposes.

### Example 2

### 7-Iodo-5-phenyl-pyrazolo[1,5-a]pyridine-2-carboxylic Acid (thiophen-2-ylmethyl)-amide (Compound 102) and

### 3,7-diiodo-5-phenyl-pyrazolo[1,5-a]pyridine-2-carboxylic Acid (thiophen-2-ylmethyl)-amide (Compound 119)

### Step 1: 1-Amino-4-phenyl-pyridinium 2,4-dinitro-phenolate

A mixture of 4-phenyl pyridine (1.55 g, 10 mmol) and 2,4-dinitro-phenyl-hydroxylamine (2.86 g, 11.5 mmol) was stirred in acetonitrile (15 mL) at 45° C. for 12.5 hours. Upon cooling, the mixture was triturated with diethyl ether (50 mL) and centrifuged to give a solid. The solid was triturated again with diethyl ether (5 mL), centrifuged and dried under high vacuum to give 1-amino-4-phenyl-pyridinium 2,4-dinitro-phenolate (3.08 g, 87%) as a yellow solid. ¹H NMR (d₆-DMSO, 300 MHz) δ 6.29 (d, 1H, J=9.8 Hz), 7.59-7.63 (m, 3H), 7.75 (dd, 1H, J=3.2, 9.7 Hz), 7.95-7.98 (m, 2H), 8.34-8.38 (m, 4H), 8.57 (d, 1H, J=3.2 Hz), 8.76-8.80 (m, 2H); MS (ESI) m/z=171 (M⁺).

### Step 2: 5-Phenyl-pyrazolo[1,5-a]pyridine-2,3-dicarboxylic Acid Dimethyl Ester

To a mixture of 1-amino-4-phenyl-pyridinium 2,4-dinitro-phenolate (3.1 g, 8.75 mmol) and K₂CO₃ (2.42 g, 17.50 mmol) in DMF (20 mL) was added dimethyl acetylenedicarboxylate (1.13 mL, 9.19 mmol) dropwise. Air was bubbled through the reaction mixture. After 2.5 hours, the solid was filtered followed by concentration of solvent under reduced pressure. The crude material was diluted with water (60 mL) and extracted with diethyl ether (3×60 mL). The combined organic extracts were dried (MgSO₄), filtered and concentrated. Column chromatography [n-hex:EtOAc (2:1) followed by n-hex:EtOAc (3:2)] of the crude gave 5-phenyl-pyrazolo[1,5-a]pyridine-2,3-dicarboxylic acid dimethyl ester (1.64 g, 60%) as a yellow solid. ¹H NMR (d₆-DMSO, 300 MHz) δ 3.86 (s, 3H), 3.93 (s, 3H), 7.47-7.59 (m, 3H), 7.62 (dd, 1H, J=2, 7.3 Hz), 7.82-7.87 (m, 2H), 8.24 (dd, 1H, J=0.9, 2 Hz), 8.97 (dd, 1H, J=0.9, 7.3 Hz); MS (ESI) m/z=333 (MNa⁺).

### Step 3: 5-Phenyl-pyrazolo[1,5-a]pyridine-2-carboxylic Acid

A solution of 5-phenyl-pyrazolo[1,5-a]pyridine-2,3-dicarboxylic acid dimethyl ester (6.33 g, 20.4 mmol) in H₂SO₄ (100 mL) and water (20 mL) was heated at 90° C. for 27 hours. The mixture was cooled to room temperature followed by the addition of water to precipitate the product. The solid was filtered, washed with water and dried under high vacuum overnight to give 5-phenyl-pyrazolo[1,5-a]pyridine-2-carboxylic acid (4.6 g, 95%) as a solid.

### Step 4: 7-Iodo-5-phenyl-pyrazolo[1,5-a]pyridine-2-carboxylic acid and 3,7-diiodo-5-phenyl-pyrazolo[1,5-a]pyridine-2-carboxylic Acid

To a solution of 5-phenyl-pyrazolo[1,5-a]pyridine-2-carboxylic acid (600 mg, 2.52 mmol) in THF (35 mL) at −78° C. was added dropwise a solution of n-butyl lithium (2.5 M in hexanes, 2.22 mL, 5.54 mmol) over 5 min. After 30 min at −78° C., a solution of iodine (1.278 g, 5.04 mmol) in THF (20 mL) was added. After 15 min, the reaction was allowed to stir at 0° C. for 30 min. An aqueous solution of sodium thiosulfate (1 M, 30 mL) was added slowly to the reaction followed by hydrochloric acid (2 N, 10 mL). The mixture was extracted with EtOAc (2×125 mL). The oragnic extracts were dried (MgSO₄), filtered and concentrated to give a mixture of acids (1.25 g) which was used for the next step without further purification.

### Step 5: 7-Iodo-5-phenyl-pyrazolo[1,5-a]pyridine-2-carboxylic Acid (thiophen-2-ylmethyl)-amide (Compound 102) and

### 3,7-diiodo-5-phenyl-pyrazolo[1,5-a]pyridine-2-carboxylic acid (thiophen-2-ylmethyl)-amide (Compound 119)

A mixture of 7-iodo-5-phenyl-pyrazolo[1,5-a]pyridine-2-carboxylic acid and 3,7-diiodo-5-phenyl-pyrazolo[1,5-a]pyridine-2-carboxylic acid (1.25 g), 2-thiophenemethylamine (0.284 mL, 2.77 mmol), N,N-di-isopropylethylamine (DIPEA, 1.32 mL, 7.56 mmol), and bromotripyrrolidinophosphonium hexafluorophosphate (PyBroP®, 1.23 g, 2.64 mmol) was stirred in DMF (25 mL) at room temperature for 30 min. The mixture was diluted with EtOAc (250 mL) and washed successively with 2N HCl (2×40 mL), saturated aqueous NaHCO₃ (40 mL), and brine (40 mL). The organic phase was dried (MgSO₄), filtered and concentrated. The crude products were column chromatographed [n-hex/EtOAc (5:1 v/v) to n-hex/EtOAc (3.5:1 v/v)] to give 3,7-diiodo-5-phenyl-pyrazolo[1,5-a]pyridine-2-carboxylic acid (thiophen-2-ylmethyl)-amide (43.2 mg, 3%) followed by 7-iodo-5-phenyl-pyrazolo[1,5-a]pyridine-2-carboxylic acid (thiophen-2-ylmethyl)-amide (468.1 mg, 40%).

Data for 7-iodo-5-phenyl-pyrazolo[1,5-a]pyridine-2-carboxylic acid (thiophen-2-ylmethyl)-amide: ¹H NMR (d₆-DMSO, 300 MHz) δ 4.66 (d, 2H, J=6.2 Hz), 6.96 (dd, 1H, J=3.2, 5 Hz), 7.04 (dd, 1H, J=1.2, 3.5 Hz), 7.28 (s, 1H), 7.39 (dd, 1H, J=1.2, 5 Hz), 7.41-7.53 (m, 2H), 7.80-7.84 (m, 2H), 7.94 (d, 1H, J=1.8 Hz), 8.13 (d, 1H, J=2.1 Hz), 8.97 (t, 1H, J=6.2 Hz); MS (ESI) m/z=460 (MH⁺).

Data for 3,7-diiodo-5-phenyl-pyrazolo[1,5-a]pyridine-2-carboxylic acid (thiophen-2-ylmethyl)-amide: ¹H NMR (d₆-DMSO, 300 MHz) δ 4.66 (d, 2H, J=6.2 Hz), 6.97 (dd, 1H, J=3.5, 5 Hz), 7.06 (dd, 1H, J=1.2, 3.5 Hz), 7.40 (dd, 1H, J=1.5, 5 Hz), 7.42-7.54 (m, 3H), 7.74 (d, 1H, J=2 Hz), 7.84-7.88 (m, 2H), 8.00 (d, 1H, J=2 Hz), 8.96 (t, 1H, J=6.2 Hz); MS (ESI) m/z=586 (MH⁺).

### Example 3

### 5-Phenyl-7-trifluoromethyl-3H-imidazo[4,5-b]pyridine-2-carboxylic acid (thiophen-2-ylmethyl)-amide (Compound 103)

### Step 1: 3-Nitro-6-phenyl-4-trifluoromethyl-pyridin-2-ylamine

(ref: D. G. Batt, G. C. Houghton, J. Het. Chem., 1995, 32,963)

Following the literature procedure, 4,4,4-trifluoromethyl-1-phenyl-1,3-butanedione (1.69 g, 7.81 mmol) and nitroacetamidine (805 mg, 7.81 mmol) was heated in EtOH (40 mL) at 95° C. for 4 days. Concentration of the solvent followed by addition of CH₂Cl₂/EtOAc/MeOH to precipitate unreacted starting material. The suspension was centrifuged and the solvent decanted and absorbed on silica gel. Column chromatography [toluene/n-hex/EtOAc (40:60:4 v/v)] of the crude product gave 3-nitro-6-phenyl-4-trifluoromethyl-pyridin-2-ylamine (515.8 mg, 23%) as a yellow solid. ¹H NMR (d₆-DMSO, 300 MHz) δ 7.48-7.56 (m, 4H), 7.60 (brs, 2H), 8.12-8.15 (m, 2H); MS (ESI) m/z=284 (MH⁺).

### Step 2: 5-Phenyl-7-trifluoromethyl-3H-imidazo[4,5-b]pyridine-2-carboxylic Acid Methyl Ester

A suspension of 3-nitro-6-phenyl-4-trifluoromethyl-pyridin-2-ylamine (513.7 mg, 18.14 mmol) and Pd/C (10%, 48 mg) in EtOH/THF (1:1 v/v, 40 mL) was shaken under H₂ atmosphere at 50 psi using a Parr apparatus for 7 hours. The catalyst was filtered through a small pad of Celite and the solvent removed under reduced pressure to give the desired product as a light orange oil (499 mg). The diaminopyridine was used for the next step without further purification. A mixture of diaminopyridine (495 mg) and methyl trimethoxyacetate (1.2 mL) (prepared according to literature: W. Kentlchner, et al, Liebigs Ann. Chem., 1980, 1448-1454) at 100° C. for 20 hours. A second batch of methyl trimethoxyacetate (0.2 mL) was added and the mixture was heated at 120° C. for 5.5 hours. The solvent was concentrated, and refluxed with charcoal (950 mg) in acetone (50 mL) for 4 hours. Upon cooling, the charcoal was filtered and the solvent concentrated. Column chromatography [n-hex/EtOAc (1:1 v/v) to n-hex/EtOAc (1:1.5 v/v)] of the crude material gave 5-phenyl-7-trifluoromethyl-3H-imidazo[4,5-b]pyridine-2-carboxylic acid methyl ester (164.6 mg, 28% yield) as a light yellow solid. ¹H NMR (d₆-DMSO, 300 MHz) δ 4.07 (s, 3H), 7.44-7.55 (m, 3H), 8.17-8.20 (m, 3H); MS (ESI) m/z=322.2 (MH⁺).

### Step 3: 5-Phenyl-7-trifluoromethyl-3H-imidazo[4,5-b]pyridine-2-carboxylic Acid (thiophen-2-ylmethyl)-amide

A mixture of 5-phenyl-7-trifluoromethyl-3H-imidazo[4,5-b]pyridine-2-carboxylic acid methyl ester (22.5 mg, 0.07 mmol) and LiOH.H₂O (29.4 mg, 0.7 mmol) was heated in THF/H₂O (3:1 v/v, 4 mL) under microwave conditions at 150° C. for 12 min. The organic solvent was removed and the mixture was acidified with 5N HCl. The aqueous solution was extracted with EtOAc (2×10 mL), dried (MgSO₄), filtered and concentrated to give the acid (26.7 mg) as a light yellow solid which was used without further purification. A mixture of the crude acid (22 mg, 0.0716 mmol), 2-thiophenemethylamine (8.1 μL, 0.079 mmol), N,N-di-isopropylethylamine (37.4 μL, 0.215 mmol), and bromotripyrrolidinophosphonium hexafluorophosphate (PyBroP®, 36.7 mg, 0.079 mmol) was stirred in DMF (1 mL) at room temperature for 45 min. The mixture was diluted with EtOAc (20 mL) and washed successively with 2N HCl (2×10 mL), saturated aqueous NaHCO₃ (10 mL), and brine (10 mL). The organic phase was dried (MgSO₄), filtered and concentrated. Column chromatography [n-hex/EtOAc (3:1 v/v)] of the crude material gave 5-phenyl-7-trifluoromethyl-3H-imidazo[4,5-b]pyridine-2-carboxylic acid (thiophen-2-ylmethyl)-amide (11.2 mg, 40%) as a light yellow powder. ¹H NMR (d₆-DMSO, 300 MHz) δ 4.78 (d, 2H, J=6.6 Hz), 6.93 (dd, 1H, J=3.5, 4.8 Hz), 7.09 (dd, 1H, J=0.9, 3.5 Hz), 7.36 (dd, 1H, J=0.9, 3.5 Hz), 7.36 (dd, 1H, J=1, 4.8 Hz), 7.40-7.53 (m, 3H), 7.97 (s, 1H), 8.11-8.14 (m, 2H), 8.92 (t, 1H, J=6.6 Hz); MS (ESI) m/z=403 (MH⁺).

### Example 4

### 3-Chloro-5-phenyl-7-trifluoromethyl-1H-indole-2-carboxylic Acid (thiophen-2-ylmethyl)-amide (Compound 104)

### Step 1: 3-Chloro-7-trifluoromethyl-1H-indole-2-carboxylic Acid

A mixture of 7-(trifluoromethyl)-1H-indole-2-carboxylic acid (1.34 g, 5.86 mmol), and N-chlorosuccinimide (939 mg, 7.03 mmol) was stirred in CHCl₃/ACN/DMF (25 mL/25 mL/5 mL) at room temperature. After 3 hours, the solvents were removed and diluted with EtOAc (150 mL), washed with 1M sodium thiosulfate (40 mL), dried (MgSO₄), filtered and concentrated to give 3-chloro-7-trifluoromethyl-1H-indole-2-carboxylic acid (2.15 g) as a brown solid. ¹H NMR (d₆-DMSO, 300 MHz) δ 3.937.36 (t, 1H, J=7.6 Hz), 7.73 (d, 1H, J=7.3 Hz), 7.93 (d, 1H, J=7 Hz), 11.04 (brs, 1H), 12.13 (s, 1H).

### Step 2: 3-Chloro-7-trifluoromethyl-1H-indole-2-carboxylic acid methyl ester

A mixture of 3-chloro-7-trifluoromethyl-1H-indole-2-carboxylic acid (1.84 g, 6.97 mmol) and conc. H₂SO₄ (0.5 mL) was heated under refux in MeOH (60 mL). After 16 hours, extra conc. H₂SO₄ (0.5 mL) and MeOH (25 mL) were added. After 2 hours, the solvent was removed and diluted with EtOAc (200 mL) and washed with saturated aqueous NaHCO₃ (50 mL), then brine (50 mL). The organic phase was filtered through a pad of silica gel, and the filtrate was concentrated. Column chromatography of the crude gave 3-chloro-7-trifluoromethyl-1H-indole-2-carboxylic acid methyl ester (583.6 mg) as an off-white solid. ¹H NMR (d₆-DMSO, 300 MHz) δ 3.93 (s, 3H), 7.37 (dt, 1H, J=0.8, 7.5 Hz), 7.76 (d, 1H, J=7.3 Hz), 7.95 (d, 1H, J=7.2 Hz), 12.30 (s, 1H); MS (ESI) m/z=278 (MHI).

### Step 3: 3-Chloro-5-iodo-7-trifluoromethyl-H-indole-2-carboxylic Acid Methyl Ester

Iodine (43.2 mg, 0.17 mmol) and sodium periodate (12.2 mg, 0.057 mmol) were dissolved in conc. H₂SO₄ (2 mL) with sonication for 15 min and stirred for extra 15 min. The iodinating reagent was then added dropwise to 3-chloro-7-trifluoromethyl-1H-indole-2-carboxylic acid methyl ester in cone. H₂SO₄ (1 mL) over 10 min. After 30 min, the reaction mixture was poured into ice-water (˜20 mL) to precipitate the product which was collected by centrifugation. The precipitate was diluted with EtOAc and passed through a small plug and concentrated to give 3-chloro-5-iodo-7-trifluoromethyl-1H-indole-2-carboxylic acid methyl ester (95.6 mg). ¹H NMR (d₆-DMSO, 300 MHz) δ 3.93 (s, 3H), 7.94 (s, 1H), 8.26 (s, 1H), 12.60 (s, 1H).

### Step 4: 3-Chloro-5-phenyl-7-trifluoromethyl-1H-indole-2-carboxylic Acid

A mixture of 3-chloro-5-iodo-7-trifluoromethyl-1H-indole-2-carboxylic acid methyl ester (92 mg, 0.228 mmol), phenylboronic acid (83.4 mg, 0.684 mmol), and tetrakis(triphenylphosphine)palladium(0) (Pd(PPh₃)₄, 5 mol %) was heated in 1M K₃PO₄ (1 mL) and 1,4-dioxane (3 mL) at 140° C. for 10 min under microwave conditions. The black precipitate was filtered, diluted with EtOAc (25 mL) and washed with saturated aqueous NaHCO₃ (15 mL), then brine (15 mL). The organic extracts were filtered through a small pad of silica gel and the solvent was removed under reduced pressure. Column chromatography of the crude material gave 3-chloro-5-phenyl-7-trifluoromethyl-1H-indole-2-carboxylic acid (41.3 mg). ¹H NMR (d₆-DMSO, 300 MHz) δ 7.26-8.19 (m, 7H); MS (ESI) m/z=340 (MH⁺).

### Step 5: 3-Chloro-5-phenyl-7-trifluoromethyl-1H-indole-2-carboxylic Acid (thiophen-2-ylmethyl)-amide (Compound 104)

3-Chloro-5-phenyl-7-trifluoromethyl-1H-indole-2-carboxylic acid and 2-thiophenemethylamine was coupled under standard amide coupling conditions to give

3-Chloro-5-phenyl-7-trifluoromethyl-1H-indole-2-carboxylic acid (thiophen-2-ylmethyl)-amide. ¹H NMR (d₆-DMSO, 300 MHz) δ 4.71 (d, 2H, J=5.9 Hz), 6.99 (dd, 1H, J=3.5, 5 Hz), 7.10 (dd, 1H, J=1.2, 3.2 Hz), 7.36-7.51 (m, 3H), 7.44 (dd, 1H, J=1.2, 5 Hz), 7.76-7.80 (m, 2H), 7.92 (brs, 1H), 8.08 (brs, 1H), 9.16 (t, 1H, J=6.2 Hz), 12.00 (s, 1H); MS (ESI) m/z=435 (MH⁺).

### Example 5

### 7-Chloro-5-furan-2-yl-1H-indole-2-carboxylic acid (thiophen-2-ylmethyl)-amide (Compound 105)

Prepared using similar procedure for compound 106

¹H NMR (d₆-DMSO, 300 MHz) δ 4.68 (d, 2H, J=5.9 Hz), 6.57 (dd, 1H, J=1.8, 3.5 Hz), 6.95 (dd, 1H, J=0.6, 3.2 Hz), 6.98 (dd, 1H, J=3.5, 5.3 Hz), 7.06 (dd, 1H, J=1.2, 3.5 Hz), 7.25 (d, 1H, J=2 Hz), 7.41 (dd, 1H, J=1.2, 5 Hz), 7.66 (d, 1H, J=1.5 Hz), 7.71 (dd, 1H, J=0.6, 1.8 Hz), 7.92 (d, 1H, J=1.2 Hz), 9.19 (t, 1H, J=5.9 Hz), 11.83 (s, 1H); MS (ESI) m/z=357, 359 (MH⁺).

### Example 6

### 7-Chloro-5-phenyl-1H-indole-2-carboxylic Acid (thiophen-2-ylmethyl)-amide (Compound 106)

### Step 1: 5-Bromo-7-chloro-1H-indole-2-carboxylic Acid (thiophen-2-ylmethyl)-amide

A mixture of 5-bromo-7-chloroindole-2-carboxylic acid (1.02 g, 3.71 mmol), 2-thiophenemethylamine (418.5 μL, 4.08 mmol), N,N-di-isopropylethylamine (1.94 mL, 11.12 mmol), and PyBroP® (1.90 g, 4.08 mmol) was stirred in DMF (15 mL) at room temperature for min. The mixture was diluted with EtOAc (150 mL) and washed successively with 2N HCl (2×50 mL), saturated aqueous NaHCO₃ (50 mL), and brine (50 mL). The organic phase was dried (MgSO₄), and filtered through a small pad of silica gel. Concentration of the solvent gave 5-bromo-7-chloro-1H-indole-2-carboxylic acid (thiophen-2-ylmethyl)-amide (1.50 g) as a white solid which was used for the next step without further purification. ¹H NMR (d₆-DMSO, 300 MHz) δ 4.67 (d, 2H, J=5.9 Hz), 6.97 (dd, 1H, J=3.5, 5 Hz), 7.06 (dd, 1H, J=1.2, 3.5 Hz), 7.19 (s, 1H), 7.41 (dd, 1H, J=1.2, 5 Hz), 7.46 (d, 1H, J=1.5 Hz), 7.86 (d, 1H, J=1.5 Hz), 9.21 (t, 1H, J=5.9 Hz), 11.98 (s, 1H); MS (ESI) m/z=368.9, 370.9 (MH⁺).

### Step 2: 7-Chloro-5-phenyl-1H-indole-2-carboxylic Acid (thiophen-2-ylmethyl)-amide (Compound 106)

A mixture of 5-bromo-7-chloro-1H-indole-2-carboxylic acid (thiophen-2-ylmethyl)-amide (200 mg, 0.541 mmol), phenylboronic acid (119 mg, 0.974 mmol) and Pd(PPh₃)₄ in aq K₃PO₄ (1M, 1 mL) and 1,4-dioxane (3 mL) was heated at 100° C. under microwave conditions for 10 min. The mixture was filtered, diluted with EtOAc (30 mL) and washed with saturated aq (15 mL), then brine (15 mL). The phase was dried (MgSO₄), filtered and concentrated. Column chromatography [n-hex/EtOAc (4:1 v/v)] of the crude material followed by crystallization from EtOAc/n-hex gave 7-chloro-5-phenyl-1H-indole-2-carboxylic acid (thiophen-2-ylmethyl)-amide (97.9 mg, 49%) as a white powder. ¹H NMR (d₆-DMSO, 300 MHz) δ 4.69 (d, 2H, J=5.6 Hz), 6.98 (dd, 1H, J=3.5, 5 Hz), 7.07 (dd, 1H, J=1.2, 3.5 Hz), 7.27 (d, 1H, J=2 Hz), 7.33 (tt, 1H, J=2, 7.3 Hz), 7.42 (dd, 1H, J=1.2, 5 Hz), 7.42-7.47 (m, 2H), 7.59 (d, 1H, J=1.5 Hz), 7.67-7.71 (m, 2H), 7.90 (d, 1H, J=1.2 Hz), 9.19 (t, 1H, J=6 Hz), 11.78 (brs, 1H); MS (ESI) m/z=367.0, 369.0 (MH⁺).

### Example 7

### 5-Phenyl-7-trifluoromethyl-pyrazolo[1,5-a]pyridine-2-carboxylic Acid (thiophen-2-ylmethyl)-amide (Compound 107)

A mixture of 7-iodo-5-phenyl-pyrazolo[1,5-a]pyridine-2-carboxylic acid (thiophen-2-ylmethyl)-amide (100 mg, 0.22 mmol), methyl 2-chloro-2,2-difluoroacetate (53.4 μL, 0.50 mmol), copper(I) iodide (50 mg, 0.26 mmol), and potassium fluoride (15.2 mg, 0.26 mmol) was stirred in DMF (0.6 mL) at 125-130° C. for 15 hours in a sealed tube. Upon cooling, the mixture was diluted with EtOAc (20 mL) and washed with saturated aqueous NH₄Cl (10 mL), then brine (10 mL). The organic layer was dried (MgSO₄), filtered and concentrated. Column chromatography [toluene/THF (98:2 v/v) to toluene/THF (96:4 v/v)] of the crude oil gave 5-phenyl-7-trifluoromethyl-pyrazolo[1,5-a]pyridine-2-carboxylic acid (thiophen-2-ylmethyl)-amide (11.7 mg, 13%) as a white solid. ¹H NMR (d₆-DMSO, 300 MHz) δ 4.66 (d, 2H, J=5.9 Hz), 6.96 (dd, 1H, J=3.5, 5 Hz), 7.04 (dd, 1H, J=1.2, 3.5 Hz), 7.28 (s, 1H), 7.39 (dd, 1H, J=1.2, 5 Hz), 7.44-7.56 (m, 3H), 7.87-7.94 (m, 3H), 8.44 (d, 1H, J=1.8 Hz), 9.02 (t, 1H, J=5.9 Hz); MS (ESI) m/z=402 (MH⁺).

### Example 8

### 7-Cyano-5-phenyl-pyrazolo[1,5-a]pyridine-2-carboxylic acid (thiophen-2-ylmethyl)-amide (Compound 108)

A mixture of 7-iodo-5-phenyl-pyrazolo[1,5-a]pyridine-2-carboxylic acid (thiophen-2-ylmethyl)-amide (62 mg, 0.135 mmol), copper (I) cyanide (48.4 mg, 0.54 mmol), 1,1′-bis(diphenylphosphino)ferrocene (dppf, 12 mg, 0.0216 mmol), and tris(dibenzylideneacetone)dipalladium(0) (Pd₂ (dba)₃, 4.9 mg, 0.0054 mmol) was heated in 1,4-dioxane (1 mL) and DMF (0.4 mL) at 135° C. for 45 min under microwave conditions. The mixture was diluted with EtOAc (20 mL) and washed with water (10 mL), dried (MgSO4), filtered and concentrated. Column chromatography [toluene/THF (98:2 v/v) to toluene/THF (96:4 v/v) of crude material gave 7-cyano-5-phenyl-pyrazolo[1,5-a]pyridine-2-carboxylic acid (thiophen-2-ylmethyl)-amide (12.9 mg, 27%) as a white powder. ¹H NMR (d₆-DMSO, 300 MHz) δ 4.65 (d, 2H, J=6 Hz), 6.96 (dd, 1H, J=3.5, 5 Hz), 7.03 (dd, 1H, J=1.2, 3.5 Hz), 7.27 (s, 1H), 7.38 (dd, 1H, J=1.2, 5 Hz), 7.43-7.56 (m, 3H), 7.86-7.90 (m, 2H), 8.40 (d, 1H, J=1.8 Hz), 8.49 (d, 1H, J=1.8 Hz), 9.18 (t, 1H, J=6 Hz); MS (ESI) m/z=359.1 (MH⁺).

### Example 10

### 3,7-Dichloro-5-phenyl-1H-indole-2-carboxylic acid (thiophen-2-ylmethyl)-amide (Compound 110)

A mixture of 7-chloro-5-phenyl-1H-indole-2-carboxylic acid (thiophen-2-ylmethyl)-amide (15.4 mg, 0.042 mmol), and N-chlorosuccinimide (7.3 mg, 0.0546 mmol) was heated in DMF (1.5 mL) at 50° C. for 1 day. The mixture was diluted with EtOAc (25 mL) and washed with aqueous sodium thiosulfate (1M, 6 mL), then brine (10 mL). The organic phase was dried (MgSO₄), filtered and concentrated. Column chromatography [n-hex/EtOAc (6:1 v/v)] of the crude material gave 3,7-dichloro-5-phenyl-1H-indole-2-carboxylic acid (thiophen-2-ylmethyl)-amide (11.9 mg, 71%) as a white solid. ¹H NMR (d₆-DMSO, 300 MHz) δ 4.71 (d, 2H, J=5.9 Hz), 6.99 (dd, 1H, J=3.5, 5 Hz), 7.10 (dd, 1H, J=1.2, 3.5 Hz), 7.34-7.49 (m, 3H), 7.43 (dd, 1H, J=1.5, 5 Hz), 7.71-7.76 (m, 4H), 8.95 (t, 1H, J=5.9 Hz), 12.13 (s, 1H); MS (ESI) m/z=401, 403 (MH⁺).

### Example 11

### 7-Bromo-5-phenyl-pyrazolo[1,5-a]pyridine-2-carboxylic Acid (thiophen-2-ylmethyl)-amide (Compound III) and

### 3,7-dibromo-5-phenyl-pyrazolo[1,5-a]pyridine-2-carboxylic Acid (thiophen-2-ylmethyl)-amide (Compound 113)

To a solution of 5-phenyl-pyrazolo[1,5-a]pyridine-2-carboxylic acid (305 mg, 1.28 mmol) in THF (18 mL) at −78° C. was added a solution of n-butyl lithium (2.5 M in hexanes, 1.13 mL, 2.83 mmol). After 30 min, a solution of 1,2-dibromotetrachloroethane (834 mg, 2.56 mmol) in THF (8 mL) was added dropwise to the reaction mixture. After 30 min, the mixture was allowed to stir at 0° C. After 1 hour, the reaction was quenched by the slow addition of 2N HCl (15 mL). The mixture was extracted with EtOAc (50 mL, 25 mL). The organic phase was dried (MgSO₄), filtered and concentrated to give a crude yellow solid (514.9 mg) which was used for the next step without further purification. The crude acids (514.9 mg), 2-thiophenemethylamine (158 L, 1.54 mmol), N,N-di-isopropylethylamine (669 μL, 3.84 mmol), and PyBroP® (657 mg, 1.41 mmol) was stirred in DMF (15 mL) at room temperature. After 30 min, the mixture was diluted with EtOAc (150 mL) and washed successively with 2N HCl (2×mL), saturated aqueous NaHCO₃ (30 mL), and brine (30 mL). The organic phase was dried (MgSO₄), filtered and concentrated. The crude products were column chromatographed [n-hex/EtOAc (5:1 v/v) to n-hex/EtOAc (3.5:1 v/v)] to give 3,7-dibromo-5-phenyl-pyrazolo[1,5-a]pyridine-2-carboxylic acid (thiophen-2-ylmethyl)-amide (46.2 mg, 7%) followed by 7-bromo-5-phenyl-pyrazolo[1,5-a]pyridine-2-carboxylic acid (thiophen-2-ylmethyl)-amide (133.7 mg, 25%).

Data for 7-bromo-5-phenyl-pyrazolo[1,5-a]pyridine-2-carboxylic acid (thiophen-2-ylmethyl)-amide: ¹H NMR (d₆-DMSO, 300 MHz) δ 4.66 (d, 2H, J=6 Hz), 6.96 (dd, 1H, J=3.5, Hz), 7.04 (dd, 1H, J=1.2, 3.5 Hz), 7.25 (s, 1H), 7.39 (dd, 1H, J=1.2, 5 Hz), 7.40-7.54 (m, 3H), 7.83-7.88 (m, 2H), 8.18 (d, 1H, J=1.8 Hz), 9.04 (t, 1H, J=6 Hz); MS (ESI) m/z=412, 414 (MH⁺).

Data for 3,7-dibromo-5-phenyl-pyrazolo[1,5-a]pyridine-2-carboxylic acid (thiophen-2-ylmethyl)-amide: ¹H NMR (d₆-DMSO, 300 MHz) δ 4.65 (d, 2H, J=6 Hz), 6.97 (dd, 1H, J=3.5, 5 Hz), 7.05 (dd, 1H, J=1.5, 3.5 Hz), 7.40 (dd, 1H, J=1.5, 5 Hz), 7.43-7.55 (m, 3H), 7.90 (d, 1H, J=2 Hz), 7.90-7.93 (m, 2H), 7.96 (d, 1H, J=2 Hz), 9.10 (t, 1H, J=6 Hz); MS (ESI) m/z=490, 492 (MH⁺).

### Example 12

### 7-Bromo-3-chloro-5-phenyl-pyrazolo[1,5-a]pyridine-2-carboxylic Acid (thiophen-2-ylmethyl)-amide (Compound 112)

A solution of 7-bromo-5-phenyl-pyrazolo[1,5-a]pyridine-2-carboxylic acid (thiophen-2-ylmethyl)-amide (36.8 mg, 0.0893 mmol) and NCS (14.3 mg, 0.107 mmol) was stirred in DMF (1 mL) at 50° C. for 4 hours. The mixture was diluted with EtOAc (20 mL) and washed with aqueous sodium thiosulfate (1M, 5 mL), then brine (5 mL). The organic phase was dried (MgSO₄), filtered and concentrated. Column chromatography [n-hex/EtOAc (7:1 v/v) to n-hex/EtOAc (5:1 v/v)] of the crude product followed by crystallization from EtOAc/n-hex gave 7-bromo-3-chloro-5-phenyl-pyrazolo[1,5-a]pyridine-2-carboxylic acid (thiophen-2-ylmethyl)-amide as a white powder (15 mg, 38%); ¹H NMR (d₆-DMSO, 300 MHz) δ 4.65 (d, 2H, J=6 Hz), 6.97 (dd, 1H, J=3.5, 5 Hz), 7.05 (dd, 1H, J=1, 3.5 Hz), 7.40 (1, 5 Hz), 7.45-7.55 (m, 3H), 7.90-7.94 (m, 2H), 7.97-8.00 (m, 2H), 9.10 (t, 1H, J=6 Hz); MS (ESI) m/z=446, 447.9 (MH⁺).

### Example 14

### 7-Methyl-5-phenyl-pyrazolo[1,5-a]pyridine-2-carboxylic acid (thiophen-2-ylmethyl)-amide (Compound 114) and

### 3,7-dimethyl-5-phenyl-pyrazolo[1,5-a]pyridine-2-carboxylic Acid (thiophen-2-ylmethyl)-amide (Compound 115)

To a stirred solution of 5-phenyl-pyrazolo[1,5-a]pyridine-2-carboxylic acid (200 mg, 0.84 mmol) in THF (15 mL) at −78° C. was added a solution of n-butyl lithium (2.5M in hexanes, 0.74 mL, 1.847 mmol) dropwise. After 30 min, methyl iodide (115 L, 1.847 mmol) was added and the mixture was allowed to slowly rise to room temperature overnight. Aqueous HCl (2N, mL) was added slowly and extracted with EtOAc (2×25 mL). The organic phase was dried (MgSO₄), filtered and concentrated to give a brown solid (236 mg) which was used for the next step without further purification. The ciude acids (236 mg), 2-thiophenemethylamine (103 μL, 1.007 mmol), N,N-di-isopropylethylamine (439 L, 2.52 mmol), and PyBroP® (430 mg, 0.923 mmol) was stirred in DMF (10 mL) at room temperature. After 1 hour, the mixture was diluted with EtOAc (125 mL) and washed successively with 2N HCl (2×25 mL), saturated aqueous NaHCO₃ (25 mL), and brine (25 mL). The organic phase was dried (MgSO₄), filtered and concentrated. The crude products were column chromatographed [n-hex/EtOAc (5:1 v/v) to n-hex/EtOAc (3.5:1 v/v)] to give 3,7-dimethyl-5-phenyl-pyrazolo[1,5-a]pyridine-2-carboxylic acid (thiophen-2-ylmethyl)-amide (13.9 mg, 5%) followed by 7-methyl-5-phenyl-pyrazolo[1,5-a]pyridine-2-carboxylic acid (thiophen-2-ylmethyl)-amide (72.9 mg, 25%) both as white powder.

Data for 7-methyl-5-phenyl-pyrazolo[1,5-a]pyridine-2-carboxylic acid (thiophen-2-ylmethyl)-amide: ¹H NMR (d₆-DMSO, 300 MHz) δ 2.79 (s, 3H), 4.65 (d, 2H, J=6.2 Hz), 6.95 (dd, 1H, J=3.5, 5 Hz), 7.03 (dd, 1H, J=1.5, 3.5 Hz), 7.08 (s, 1H), 7.37 (d, 1H, J=1.5 Hz), 7.38 (dd, 1H, J=1.5, 5 Hz), 7.36-7.53 (m, 3H), 7.78-7.83 (m, 2H), 7.99 (d, 1H, J=1.5 Hz), 9.01 (t, 1H, J=6.2 Hz); MS (ESI) m/z=348.1 (MH⁺).

Data for 3,7-dimethyl-5-phenyl-pyrazolo[1,5-a]pyridine-2-carboxylic acid (thiophen-2-ylmethyl)-amide: ¹H NMR (d₆-DMSO, 300 MHz) δ 2.55 (s, 3H), 2.75 (s, 3H), 4.65 (d, 2H, J=6.2 Hz), 6.96 (dd, 1H, J=3.2, 5 Hz), 7.03 (dd, 1H, J=1.2, 3.2 Hz), 7.32 (dd, 1H, J=1.2, 2 Hz), 7.37 (dd, 1H, J=1.2, 5 Hz), 7.36-7.52 (m, 3H), 7.82-7.86 (m, 2H), 7.92 (d, 1H, J=1.5 Hz), 8.82 (t, 1H, J=6.2 Hz); MS (ESI) m/z=362.1 (MH⁺).

### Example 16

### 7-Furan-2-yl-5-phenyl-pyrazolo[1,5-a]pyridine-2-carboxylic Acid (thiophen-2-ylmethyl)-amide (Compound 116)

A mixture of 7-bromo-5-phenyl-pyrazolo[1,5-a]pyridine-2-carboxylic acid (thiophen-2-ylmethyl)-amide (38 mg, 0.0922 mmol), 2-furanboronic acid (31 mg, 0.276 mmol), tetrakis(triphenylphosphine)palladium(0) (Pd(PPh₃)₄, 5.3 mg, 0.005 mmol) was heated in aq. K₃PO₄ (1M, 0.5 mL) and 1,4-dioxane (1.5 mL) at 100° C. for 20 min under microwave conditions. The mixture was diluted with EtOAc (100 mL), and washed with saturated aqueous NaHCO₃ (20 mL), and brine (20 mL). The organic phase was dried (MgSO₄), filtered and concentrated. The crude material was column chromatographed [n-hex/EtOAc (5:1 v/v) to n-hex/EtOAc (3:1 v/v)] to give 7-furan-2-yl-5-phenyl-pyrazolo[1,5-a]pyridine-2-carboxylic acid (thiophen-2-ylmethyl)-amide (24.9 mg, 68%).). ¹H NMR (d₆-DMSO, 300 MHz) δ 4.71 (d, 2H, J=6.2 Hz), 6.86 (dd, 1H, J=1.8, 3.5 Hz), 6.97 (dd, 1H, J=3.5, 5 Hz), 7.06 (dd, 1H, J=1.2, 3.5 Hz), 7.21 (s, 1H), 7.39 (dd, 1H, J=1.2, 5 Hz), 7.42-7.57 (m, 3H), 7.81 (d, 1H, J=1.8 Hz), 7.85-7.88 (m, 2H), 8.05 (d, 1H, J=1.2 Hz), 8.11 (d, 1H, J=1.8 Hz), 8.41 (d, 1H, J=3.5 Hz), 9.33 (t, 1H, J=6.2 Hz); MS (ESI) m/z=400.1 (MH⁺).

### Example 17

### 7-Methoxy-5-phenyl-pyrazolo[1,5-a]pyridine-2-carboxylic Acid (thiophen-2-ylmethyl)-amide (Compound 117)

A mixture of 7-bromo-5-phenyl-pyrazolo[1,5-a]pyridine-2-carboxylic acid (thiophen-2-ylmethyl)-amide (16 mg, 0.0388 mmol), sodium methoxide (2.2 mg, 0.0407 mmol) in MeOH (1.3 mL) was heated at 140° C. for 40 min under microwave conditions. Additional sodium methoxide was added followed by heating at 130° C. for 1 hour under microwave condition. A solution of HCl (2M in ether, 0.5 mL) was added followed by concentration of solvent. The crude product was digested with CH₂Cl₂ followed by filtration of precipitate. The filtrate was concentrated followed by column chromatography [n-hex/EtOAc (3:2 v/v)] to give 7-methoxy-5-phenyl-pyrazolo[1,5-a]pyridine-2-carboxylic acid (thiophen-2-ylmethyl)-amide (5.6 mg, 40%). ¹H NMR (d₆-DMSO, 300 MHz) δ 4.22 (s, 3H), 4.62 (d, 2H, J=6.2 Hz), 6.79 (d, 1H, J=1.8 Hz), 6.94 (dd, 1H, J=3.5, 5 Hz), 7.01 (dd, 1H, J=1.2, 3.5 Hz), 7.01 (s, 1H), 7.37 (dd, 1H, J=1.2, 5 Hz), 7.39-7.54 (m, 3H), 7.69 (d, 1H, J=1.8 Hz), 7.82-7.86 (m, 2H), 9.08 (t, 1H, J=6.2 Hz); MS (ESI) m/z=364.1 (MH⁺).

### Example 18

### 3-Bromo-5-phenyl-7-trifluoromethyl-pyrazolo[1,5-a]pyridine-2-carboxylic Acid (thiophen-2-ylmethyl)-amide (Compound 118)

A mixture of 5-phenyl-7-trifluoromethyl-pyrazolo[1,5-a]pyridine-2-carboxylic acid (thiophen-2-ylmethyl)-amide (19.2 mg, 0.048 mmol) and NBS (8.9 mg, 0.0502 mmol) in DMF (1 mL) was heated at 45° C. for 1 hour. Upon cooling, the product was purified by preparative HPLC (40-100% ACN gradient) to give 3-bromo-5-phenyl-7-trifluoromethyl-pyrazolo[1,5-a]pyridine-2-carboxylic acid (thiophen-2-ylmethyl)-amide (10.9 mg, 47%). ¹H NMR (d₆-DMSO, 300 MHz) δ 4.65 (d, 2H, J=5.9 Hz), 6.97 (dd, 1H, J=3.5, 5 Hz), 7.05 (dd, 1H, J=1.2, 3.5 Hz), 7.40 (dd, 1H, J=1.5, 5 Hz), 7.46-7.57 (m, 3H), 7.95-7.98 (m, 2H), 8.03 (d, 1H, J=1.8 Hz), 8.15 (d, 1H, J=1.8 Hz), 9.05 (t, 1H, J=5.9 Hz); MS (ESI) m/z=480, 482 (MH⁺).

### Example 20

### 3-Bromo-7-iodo-5-phenyl-pyrazolo[1,5-a]pyridine-2-carboxylic Acid (thiophen-2-ylmethyl)-amide (Compound 120)

A mixture of 7-iodo-5-phenyl-pyrazolo[1,5-a]pyridine-2-carboxylic acid (thiophen-2-ylmethyl)-amide (40 mg, 0.087 mmol) and N-bromosuccinimide (NBS, 17 mg, 0.0958 mmol) was stirred in DMF at 40° C. for 14 hours. Upon cooling, the mixture was diluted with EtOAc (20 mL) and washed with aq. sodium thiosulfate solution (1M, 10 mL), then brine (10 mL). The organic phase was dried (MgSO₄), filtered and concentrated. The product was purified by preparative HPLC (30-100% ACN gradient) to give 3-bromo-7-iodo-5-phenyl-pyrazolo[1,5-a]pyridine-2-carboxylic acid (thiophen-2-ylmethyl)-amide as a white powder (24.1 mg, 52%). ¹H NMR (d₆-DMSO, 300 MHz) δ 4.66 (d, 2H, J=5.9 Hz), 6.97 (dd, 1H, J=3.5, 5 Hz), 7.05 (dd, 1H, J=1.2, 3.5 Hz), 7.40 (dd, 1H, J=1.5, 5 Hz), 7.45-7.54 (m, 3H), 7.85 (d, 1H, J=2 Hz), 7.86-7.90 (m, 2H), 8.04 (d, 1H, J=2 Hz), 9.02 (t, 1H, J=5.9 Hz); MS (ESI) m/z=538 (MH⁺).

### Example 21

### 3-Chloro-7-iodo-5-phenyl-pyrazolo[1,5-a]pyridine-2-carboxylic Acid (thiophen-2-ylmethyl)-amide (Compound 121)

A mixture of the 7-iodo-5-phenyl-pyrazolo[1,5-a]pyridine-2-carboxylic acid (thiophen-2-ylmethyl)-amide (40.5 mg, 0.0882 mmol) and N-chlorosuccinimide (NCS, 14.1 mg, 0.106 mmol) was stirred in DMF at 40° C. for 14 hours. A second batch of NCS (4.3 mg) was added and the reaction heated at 50° C. for 1 day. Upon cooling, the mixture was purified by preparative HPLC (40-100% ACN gradient) to give 3-chloro-7-iodo-5-phenyl-pyrazolo[1,5-a]pyridine-2-carboxylic acid (thiophen-2-ylmethyl)-amide (23.7 mg, 45%) as a white solid. ¹H NMR (d₆-DMSO, 300 MHz) δ 4.66 (d, 2H, J=5.9 Hz), 6.97 (dd, 1H, J=3.5, 5 Hz), 7.05 (dd, 1H, J=1.2, 3.5 Hz), 7.40 (dd, 1H, J=1.2, 5 Hz), 7.42-7.54 (m, 3H), 7.87-7.91 (m, 2H), 7.94 (d, 1H, J=1.8 Hz), 8.05 (d, 1H, J=1.8 Hz), 9.01 (t, 1H, J=5.9 Hz); MS (ESI) m/z=494 (MH⁺).

### Example 22

### 3-Chloro-5-phenyl-7-trifluoromethyl-pyrazolo[1,5-a]pyridine-2-carboxylic Acid (thiophen-2-ylmethyl)-amide (Compound 122) and

### 3-Chloro-5-phenyl-7-trifluoromethyl-pyrazolo[1,5-a]pyridine-2-carboxylic Acid (5-chloro-thiophen-2-ylmethyl)-amide (Compound 123)

A mixture of 5-phenyl-7-trifluoromethyl-pyrazolo[1,5-a]pyridine-2-carboxylic acid (thiophen-2-ylmethyl)-amide (16.3 mg, 0.0406 mmol) and NCS (6.5 mg, 0.0487 mmol) in DMF (1 mL) was heated at 55° C. for 2.5 hours. A second batch of NCS (11 mg) was added to the reaction mixture and heated at 45° C. for 21.5 hours. Upon cooling, the product was purified by preparative HPLC (50-100% ACN gradient) to give the 3-Chloro-5-phenyl-7-trifluoromethyl-pyrazolo[1,5-a]pyridine-2-carboxylic acid (thiophen-2-ylmethyl)-amide (5.8 mg, 27%), and 3-Chloro-5-phenyl-7-trifluoromethyl-pyrazolo[1,5-a]pyridine-2-carboxylic acid (5-chloro-thiophen-2-ylmethyl)-amide (4 mg, 21%).

Data for 3-chloro-5-phenyl-7-trifluoromethyl-pyrazolo[1,5-a]pyridine-2-carboxylic acid (thiophen-2-ylmethyl)-amide: ¹H NMR (d₆-DMSO, 300 MHz) δ 4.65 (d, 2H, J=5.9 Hz), 6.97 (dd, 1H, J=3.5, 5 Hz), 7.05 (dd, 1H, J=1.2, 3.5 Hz), 7.40 (dd, 1H, J=1.2, 5 Hz), 7.45-7.57 (m, 3H), 7.95-7.99 (m, 2H), 8.03 (d, 1H, J=1.8 Hz), 8.25 (d, 1H, J=1.8 Hz), 9.05 (t, 1H, J=5.9 Hz); MS (ESI) m/z=436 (MH⁺).

Data for 3-chloro-5-phenyl-7-trifluoromethyl-pyrazolo[1,5-a]pyridine-2-carboxylic acid (5-chloro-thiophen-2-ylmethyl)-amide: ¹H NMR (d₆-DMSO, 300 MHz) δ 4.57 (d, 2H, J=6.2 Hz), 6.91 (d, 1H, J=3.5 Hz), 6.97 (d, 1H, J=3.5 Hz), 7.45-7.57 (m, 3H), 7.96-7.99 (m, 2H), 8.04 (d, 1H, J=1.8 Hz), 8.25 (d, 1H, J=1.8 Hz), 9.10 (t, 1H, J=6.2 Hz); MS (ESI) m/z=470, 472 (MH⁺).

### Example 24

### 7-Iodo-5-phenyl-pyrazolo[1,5-a]pyridine-2-carboxylic Acid (furan-2-ylmethyl)-amide (Compound 124)

Prepared using the procedure as for compound 102.

¹H NMR (d₆-DMSO, 300 MHz) δ 4.50 (d, 2H, J=5.7 Hz), 6.29 (d, 1H, J=3 Hz), 6.40 (dd, 1H, J=1.8, 3 Hz), 7.28 (s, 1H), 7.40-7.52 (m, 3H), 7.58 (s, 1H), 7.81 (m, 2H), 7.93 (d, 1H, J=1.8 Hz), 8.12 (d, 1H, J=1.8 Hz), 8.78 (t, 1H, J=5.7 Hz); MS (ESI) m/z=444 (MH⁺).

### Example 25

### 7-Iodo-5-phenyl-pyrazolo[1,5-a]pyridine-2-carboxylic Acid (2-thiophen-2-yl-ethyl)-amide (Compound 125)

Prepared using the procedure as for compound 102.

¹H NMR (d₆-DMSO, 300 MHz) δ 3.11 (t, 2H, J=7 Hz), 3.57 (q, 2H, J=7 Hz), 6.92-6.98 (m, 2H), 7.25 (s, 1H), 7.34 (dd, 1H, J=1.2, 5.4 Hz), 7.40-7.52 (m, 3H), 7.80-7.84 (m, 2H), 7.93 (d, 1H, J=1.8 Hz), 8.12 (d, 1H, J=1.8 Hz), 8.45 (t, 1H, J=7 Hz); MS (ESI) m/z=474 (MH⁺).

### Example 26

### 7-Iodo-5-phenyl-pyrazolo[1,5-a]pyridine-2-carboxylic Acid (thiophen-3-ylmethyl)-amide (Compound 126)

Prepared using the procedure as for compound 102.

¹H NMR (d₆-DMSO, 300 MHz) δ 4.45 (d, 2H, J=6.2 Hz), 7.12 (dd, 1H, J=1.3, 4.8 Hz), 7.32 (dd, 1H, J=0.9, 2.6 Hz), 7.43-7.56 (m, 5H), 7.73 (dd, 1H, J=0.9, 2.2 Hz), 7.82-7.87 (m, 2H), 8.78 (d, 1H, J=7.9 Hz), 8.99 (t, 1H, J=6.2 Hz); MS (ESI) m/z=460 (MH⁺).

### Example 27

### 7-Iodo-5-phenyl-pyrazolo[1,5-a]pyridine-2-carboxylic Acid phenylamide (Compound 127)

Prepared using the procedure as for compound 102.

¹H NMR (d₆-DMSO, 300 MHz) δ 7.11 (brt, 1H, J=7.4 Hz), 7.35 (brt, 2H, J=7.9 Hz), 7.44-7.57 (m, 4H), 7.77-7.89 (m, 5H), 8.86 (d, 1H), 10.50 (s, 1H); MS (ESI) m/z=440 (MH⁺).

### Example 28

### 7-Iodo-5-phenyl-pyrazolo[1,5-a]pyridine-2-carboxylic Acid 2-fluoro-benzylamide (Compound 128)

Prepared using the procedure as for compound 102.

¹H NMR (d₆-DMSO, 300 MHz) δ 4.57 (d, 2H, J=6.2 Hz), 7.14-7.22 (m, 2H), 7.27-7.53 (m, 6H), 7.80-7.84 (m, 2H), 7.94 (d, 1H, J=1.8 Hz), 8.13 (d, 1H, J=1.8 Hz), 8.89 (t, 1H, J=6.2 Hz); MS (ESI) m/z=472 (MH⁺).

### Example 29

### 7-Iodo-5-phenyl-pyrazolo[1,5-a]pyridine-2-carboxylic Acid benzylamide (Compound 129)

Prepared using the procedure as for compound 102.

¹H NMR (d₆-DMSO, 300 MHz) δ 4.51 (d, 2H, J=6 Hz), 7.20-7.54 (m, 9H), 7.80-7.83 (m, 2H), 7.93 (d, 1H, J=1.8 Hz), 8.13 (d, 1H, J=1.8 Hz), 8.91 (t, 1H, J=6 Hz); MS (ESI) m/z=454 (MH⁺).

### Example 30

### 7-Iodo-5-phenyl-pyrazolo[1,5-a]pyridine-2-carboxylic Acid phenethyl-amide (Compound 130)

Prepared using the procedure as for compound 102.

¹H NMR (d₆-DMSO, 300 MHz) δ 2.89 (t, 2H, J=7 Hz), 3.54 (q, 2H, J=6.2 Hz), 7.16-7.34 (m, 6H), 7.39-7.52 (m, 3H), 7.82 (d, 2H, J=7 Hz), 7.93 (d, 1H, J=1.8 Hz), 8.12 (d, 1H, J=1.8 Hz), 8.34 (t, 1H, J=6.2 Hz); MS (ESI) m/z=468 (MH⁺).

### Example 31

### 7-Iodo-5-phenyl-pyrazolo[1,5-a]pyridine-2-carboxylic Acid (tetrahydro-furan-2-ylmethyl)-amide (Compound 131)

Prepared using the procedure as for compound 102.

¹H NMR (d₆-DMSO, 300 MHz) δ 1.54-1.98 (m, 4H), 3.58-4.05 (m, 3H), 7.25 (s, 1H), 7.38-7.52 (m, 3H), 7.78-7.82 (m, 2H), 7.92 (d, 1H, J=1.8 Hz), 8.10 (d, 1H, J=1.8 Hz), 8.15 (t, 1H, J=6 Hz); MS (ESI) m/z=448 (MH⁺).

### Example 33

### 7-(Chloro-difluoro-methyl)-5-furan-2-yl-pyrazolo[1,5-a]pyridine-2-carboxylic Acid (thiophen-2-ylmethyl)-amide (Compound 133)

### Step 1: (E)-4-Furan-2-yl-2-oxo-but-3-enoic Acid

To a stirred solution of 2-furaldehyde (15 mL, 181 mmol), and pyruvic acid (12.6 mL, 181 mmol) at 0° C. was added dropwise a solution of 10% NaOH over 15 min during which a yellow cake was formed. After 10 min, the cake was poured into a 1 L flask and the cake was dissolved with water (650 mL). The solution was acidified with 10% H₂SO₄ (˜65 mL) to precipitate product. The mixture was cooled with an ice-water bath for an hour followed by filtration to give (E)-4-furan-2-yl-2-oxo-but-3-enoic acid (16.47 g, 55%) as a yellow solid. ¹H NMR (d₆-DMSO, 300 MHz) δ 6.71 (dd, 1H, J=1.8, 3.5 Hz), 6.96 (d, 1H, J=15.8 Hz), 7.17 (d, 1H, J=3.5 Hz), 7.54 (d, 1H, J=15.4 Hz), 7.95 (d, 1H, J=1.8 Hz); MS (ESI) m/z=189 (MNa⁺).

### Step 2: Pyridinium di-chlorodifluoroacetyl Methylid

To a suspension of 1-carboxymethyl-pyridinium betaine (prepared based on literature method: Thorsteinsson, et al, J. Med. Chem., 2003, 46, 4173) (15 g, 0.109 mol) in Et₂O (70 mL) was added triethylamine (TEA, 6.1 mL, 0.044 mol) followed by the dropwise addition of chlorodifluoroacetic anhydride (45.72 mL, 0.263 mol) over 25 min. After 95 min, the ice bath was removed and the mixture was allowed to stir at room temperature for 3 hours. The mixture was cooled with an ice-water bath and TEA (˜50 mL) was added to neutralize the reaction. The ethereal layer was concentrated to give a brown semi-solid which was poured into ice-water (500 mL) and stirred for 30 min. The precipitate was filtered and dried under high vacuum overnight. The crude material was crystallized from EtOAc/n-hex to give pyridinium di-chlorodifluoroacetyl methylid (23.05 g, qunatitative). ¹H NMR (d₆-DMSO, 300 MHz) δ 8.16 (m, 2H), 8.70 (tt, 1H, J=1.5 Hz, 7.6 Hz), 9.05 (d, 2H, J=5.6 Hz); MS (ESI) m/z=317.9, 320 (MH⁺).

### Step 3: Chlorodifluoromethylacylpyridinium Chloride

A suspension of pyridinium di-chlorodifluoroacetyl methylid (23.05 g, 0.13 mol) was heated in 2N HCl (300 mL) at 65° C. for 30 min. The cleared solution was concentrated under reduced pressure and triturated with water (80 mL). The precipitate was filtered and dried under reduced pressure to give chlorodifluoromethylacylpyridinium chloride (15.67 g) as beige solid. ¹H NMR (d₆-DMSO, 300 MHz) δ 4.94 (s, 2H), 7.96 (s, 2H), 8.18 (dd, 2H, J=6.7, 7.6 Hz), 8.68 (tt, 1H, J=1.6, 7.6 Hz), 8.99 (brd, 2H, J=6.7 Hz); MS (ESI) m/z=224 (M⁺).

### Step 4: 6-(Chloro-difluoro-methyl)-4-furan-2-yl-pyridine-2-carboxylic Acid

A suspension of chlorodifluoromethylacylpyridinium chloride (9.02 g, 34.68 mmol), (E)-4-furan-2-yl-2-oxo-but-3-enoic acid (5.76 g, 34.68 mmol) and ammonium acetate (21.4 g, 277.5 mmol) was heated in water (50 mL) at 95° C. for 8.5 hours. The mixture was cooled and extracted with EtOAc (200 mL, 2×100 mL), dried (MgSO₄), filtered and concentrated. The product was precipitated from toluene/n-hex (1:1 v/v, 400 mL) to give a brown precipitate (6.29 g, 66% yield). ¹H NMR (d₆-DMSO, 300 MHz) δ 6.72 (dd, 1H, J=1.8, 3.6 Hz), 7.53 (d, 1H, J=3.3 Hz), 7.94 (brd, 1H, J=1.8 Hz), 8.00 (s, 1H), 8.27 (s, 1H); MS (ESI) m/z=274 (MH⁺).

### Step 5: 6-(Chloro-difluoro-methyl)-4-furan-2-yl-pyridine-2-carboxylic Acid Methoxy-methyl-amide

To a stirred solution of 6-(chloro-difluoro-methyl)-4-furan-2-yl-pyridine-2-carboxylic acid 4.63 g, 16.92 mmol) in DMF (65 mL) was added, N,O-dimethylhydroxylamine hydrochloride (1.98 g, 20.2 mmol), N-(3-dimethylaminopropyl)-N-ethylcarbodiimide hydrochloride (EDC.HCl, 3.89 g, 20.3 mmol), 1-hydroxybenzotriazole (HOBt, 2.74 g, 20.3 mmol), and N,N-di-iso-propylethylamine (14.7 mL, 84.6 mmol). After 15 hours at room temperature, the mixture was heated at 40° C. for 8.5 hours. A second batch of N,O-dimethylhydroxylamine hydrochloride (413 mg, 4.23 mmol), EDC.HCl (811 g, 4.23 mmol), HOBt (572 mg, 4.23 mmol), and N,N-di-iso-propylethylamine (2.95 mL, 16.92 mmol) was added and the mixture was stirred for 16 hours. The mixture was cooled and O-(7-azabenzotriazol-1-yl)-N,N,N′,N′-tetramethyluronium hexafluorophosphate (HATU, 1.61 g, 4.23 mmol) was added. The mixture was heated at 50° C. for 75 min. Upon cooling, the mixture was diluted with EtOAc (650 mL) and washed successively with 2N HCl (80 mL), saturated aqueous NaHCO₃ (80 mL) and brine (80 mL). The organic phase was dried (Na₂SO₄), filtered and concentrated. The crude material was purified by column chromatography [n-hex/EtOAc (4:1) to n-hex/EtOAc (2.5:1)] to give 6-(chloro-difluoro-methyl)-4-furan-2-yl-pyridine-2-carboxylic acid methoxy-methyl-amide (3.26 g, 61%) as a white solid. ¹H NMR (d₆-DMSO, 300 MHz) δ 3.31 (s, 3H), 3.71 (s, 3H), 6.76 (dd, 1H, J=1.8, 3.5 Hz), 7.64 (d, 1H, J=3.5 Hz), 7.99 (dd, 1H, J=0.6, 1.8 Hz), 8.07 (brs, 1H), 8.13 (d, 1H, J=1.5 Hz); MS (ESI) m/z=317 (MH⁺).

### Step 6: 6-(Chloro-difluoro-methyl)-4-furan-2-yl-pyridine-2-carbaldehyde

To a stirred solution of 6-(chloro-difluoro-methyl)-4-furan-2-yl-pyridine-2-carboxylic acid methoxy-methyl-amide (2.97 g, 9.39 mmol) in THF (70 mL) at −78° C. was added dropwise a solution of diisobutylaluminum hydride (DIBAL-H, 1M in THF) (16.9 mL, 16.9 mmol). After 1.5 hours, the reaction was quenched by the careful addition of 2N HCl (15 mL). After 5 min, the mixture was allowed to stir at 0° C. for 10 min. The mixture was diluted with EtOAc (700 mL) and saturated aqueous NaHCO₃ (75 mL) and brine (35 mL). The gel was passed through a small pad of Celite and the aqueous phase was separated and extracted with EtOAc (150 mL). The combined organic extracts were dried (Na₂SO₄), filtered and concentrated. The crude material was purified by column chromatography [n-hex/EtOAc (10:1) to n-hex/EtOAc (7:1)] to give 6-(chloro-difluoro-methyl)-4-furan-2-yl-pyridine-2-carbaldehyde (2.20 g, 91%) as a white solid. ¹H NMR (d₆-DMSO, 300 MHz) δ 6.78 (dd, 1H, J=1.8, 3.5 Hz), 7.73 (dd, 1H, J=0.6, 3.5 Hz), 8.02 (dd, 1H, J=0.9, 1.8 Hz), 8.29 (d, 1H, J=1.5 Hz), 8.32 (d, 1H, J=1.5 Hz), 10.00 (s, 1H); MS (ESI) m/z=258 (MH⁺).

### Step 7: (Z)-2-Azido-3-[6-(chloro-difluoro-methyl)-4-furan-2-yl-pyridin-2-yl]-acrylic Acid Ethyl Ester

To a stirred solution of 6-(chloro-difluoro-methyl)-4-furan-2-yl-pyridine-2-carbaldehyde (52.7 mg, 0.205 mmol) in EtOH (0.8 mL) at −45° C. was added a solution of sodium ethoxide (21 wt % in EtOH, 232 μL, 0.716 mmol). A solution of tert-butyl azidoacetate (prepared according to literature Moore and Rydon, Organic Synthesis, Coll Vol 5, 586.) in EtOH (0.4 mL) was then added dropwise at −45° C. The mixture was allowed to slowly warm to −8° C. overnight. The mixture was diluted with EtOAc (30 mL) and washed with saturated aqueous NH₄Cl (10 mL), then brine (10 mL). The organic phase was dried (MgSO₄), filtered and concentrated. Purification of the crude material by preparative TLC (eluted with n-hex/EtOAc (5:1 v/v)] gave (Z)-2-azido-3-[6-(chloro-difluoro-methyl)-4-furan-2-yl-pyridin-2-yl]-acrylic acid ethyl ester (15.4 mg, 20%) as a white solid. ¹H NMR (d₆-DMSO, 300 MHz) δ 1.35 (t, 3H, J=7 Hz), 4.34 (q, 2H, J=7 Hz), 6.75 (dd, 1H, J=1.8, 3.5 Hz), 6.86 (s, 1H), 7.61 (dd, 2H, J=0.9, 3.5 Hz), 7.99 (m, 2H), 8.54 (d, 1H, J=1.2 Hz); MS (ESI) m/z=391 (MNa⁺).

### Step 8: 7-(Chloro-difluoro-methyl)-5-furan-2-yl-pyrazolo[1,5-a]pyridine-2-carboxylic Acid Ethyl Ester

A solution of (Z)-2-azido-3-[6-(chloro-difluoro-methyl)-4-furan-2-yl-pyridin-2-yl]-acrylic acid ethyl ester (32.9 mg, 0.0892 mmol) in DMF (3 mL) was heated at 180° C. for 10 min under microwave conditions. The solvent was removed under reduced pressure followed by column chromatography [n-hex/EtOAc (8:1 v/v) to n-hex/EtOAc (6:1 v/v)] to give 7-(chloro-difluoro-methyl)-5-furan-2-yl-pyrazolo[1,5-a]pyridine-2-carboxylic acid ethyl ester (10.6 mg, 35%) as an off-white solid. ¹H NMR (d₆-DMSO, 300 MHz) δ 1.36 (t, 3H, J=7 Hz), 4.39 (q, 2H, J=7 Hz), 6.71 (dd, 1H, J=1.8, 3.5 Hz), 7.36 (s, 1H), 7.42 (d, 1H, J=3.2 Hz), 7.91 (d, 1H, J=1.5 Hz), 7.93 (d, 1H, J=1.8 Hz), 8.30 (d, 1H, J=1.5 Hz); MS (ESI) m/z=341 (MH⁺).

### Step 9: 7-(Chloro-difluoro-methyl)-5-furan-2-yl-pyrazolo[1,5-a]pyridine-2-carboxylic Acid (thiophen-2-ylmethyl)-amide (Compound 133)

To a solution of 7-(chloro-difluoro-methyl)-5-furan-2-yl-pyrazolo[1,5-a]pyridine-2-carboxylic acid ethyl ester (11.5 mg, 0.0338 mmol) in THF/MeOH/H₂O (3:1:1 v/v, 1.5 mL) was added a solution of LiOH (2.5M in water, 40 μL, 0.1013 mmol). After 1 hour, the solvent was concentrated and 2N HCl (0.5 mL) was added followed by extraction with EtOAc (10 mL, 5 mL). The organic extracts were dried (Na₂SO₄), filtered and concentrated to give the acid (16.6 mg) which was used for the next step without further purification. To a stirred solution of the acid (16.6 mg) in DMF (0.8 mL) was added 2-thiophenemethylamine (5.2 μL, 0.0506 mmol), N,N-di-isopropylethylamine (23.5 μL, 0.135 mmol), and PyBroP® (19.7 mg, 0.0422 mmol). After 30 min, the mixture was diluted with EtOAc (20 mL) and washed successively with 2N HCl (2×5 mL), saturated aqueous NaHCO₃ (5 mL), and brine (5 mL). The organic phase was dried (Na₂SO₄), filtered and concentrated. The crude product was column chromatographed [n-hex/EtOAc (4:1 v/v) to n-hex/EtOAc (3:1 v/v)] to give 7-(chloro-difluoro-methyl)-5-furan-2-yl-pyrazolo[1,5-a]pyridine-2-carboxylic acid (thiophen-2-ylmethyl)-amide (11.9 mg, 86%) as a white solid. ¹H NMR (d₆-DMSO, 300 MHz) δ 4.66 (d, 2H, J=6.2 Hz), 6.70 (dd, 1H, J=1.7, 3.5 Hz), 6.95 (dd, 1H, J=3.5, 5.2 Hz), 7.03 (dd, 1H, J=1, 3.2 Hz), 7.29 (s, 1H), 7.38 (dd, 1H, J=1.4, 5.2 Hz), 7.40 (brd, 1H, J=3.2 Hz), 7.87 (d, 1H, J=2 Hz), 7.90 (d, 1H, J=1.5 Hz), 8.29 (d, 1H, J=1.8 Hz), 8.96 (t, 1H, J=6.2 Hz); MS (ESI) m/z=408 (MH⁺).

### Example 34

### 6-Bromo-8-trifluoromethyl-imidazo[1,2-a]pyridine-2-carboxylic Acid Ethyl Ester (Compound 134)

### Step 1: 5-Bromo-3-trifluoromethyl-pyridin-2-ylamine

2-Amino-3-trifluoromethylpyridine (5.4 gm, 33.3 mmol) was dissolved in DMF (31 mL) and N-bromosuccinimide (5.9 gm, 33.3 mmol) dissolved in DMF (31 mL) was added dropwise. The mixture was stirred for 4 hours, concentrated to −20 mL and added dropwise into ice-water (600 mL). The product crashed out, was filtered, washed with water (100 mL) and dried under vacuum to afford 5-bromo-3-trifluoromethyl-pyridin-2-ylamine as a light brown solid (7.12 gm, 88%). ¹H NMR (d₆-DMSO, 300 MHz) δ 8.22 (s, 1H), 7.85 (s, 1H), 6.66 (s, 2H); MS (ESI) m/z=242.9 (MH⁺).

### Step 2: 6-Bromo-8-trifluoromethyl-imidazo[1,2-a]pyridine-2-carboxylic Acid Ethyl Ester

A mixture of 5-bromo-3-trifluoromethyl-pyridin-2-ylamine (21.78 g, 90.37 mmol) and ethyl bromopyruvate (90% pure, 25.3 mL, 180.74 mmol) was heated in DMF (180 mL) at 50° C. for 1 day. Upon cooling, the solvent was removed to half the volume under reduced pressure. The mixture was diluted with EtOAc (500 mL) and washed with water (3×150 mL), dried (Na₂SO₄), filtered and concentrated. The crude brown oil was dissolved in minimum amount of EtOAc and dripped slowly into n-hexanes (500 mL) with vigorous stirring. The suspension was allowed to stir overnight and filtered to give 6-bromo-8-trifluoromethyl-imidazo[1,2-a]pyridine-2-carboxylic acid ethyl ester (26.83 g, 89%) as a yellow solid. ¹H NMR (d₆-DMSO, 300 MHz) δ 1.33 (t, 3H, J=7 Hz), 4.34 (q, 2H, J=7 Hz), 8.00 (brs, 1H), 8.60 (s, 1H), 9.16 (brs, 1H); MS (ESI) m/z=337, 339 (MH⁺).

### Example 35

### 6-Bromo-8-trifluoromethyl-imidazo[1,2-a]pyridine-2-carboxylic Acid (Compound 135)

6-Bromo-8-trifluoromethyl-imidazo[1,2-a]pyridine-2-carboxylic acid ethyl ester (1 gm, 2.96 mmol) was suspended in acetonitrile (30 mL) and HCl (2N aqueous, 20 mL) was added and the mixture refluxed over 12 hours. Upon cooling to room temperature, a white solid crystallized out and was filtered, washed (water) and dried to afford 6-bromo-8-trifluoromethyl-imidazo[1,2-a]pyridine-2-carboxylic acid (0.45 gm, 49%) as a white solid. MS (ESI) m/z=310.0 (MH⁺).

### Example 36

### 6-Bromo-8-trifluoromethyl-imidazo[1,2-a]pyridine-2-carboxylic Acid (thiophen-2-ylmethyl)-amide (Compound 136)

6-Bromo-8-trifluoromethyl-imidazo[1,2-a]pyridine-2-carboxylic acid (0.45 g, 1.47 mmol) and HBTU (0.67 g, 1.76 mmol) were dissolved in DMF (3 mL) and 2-thiophene methyl amine (0.18 g, 1.47 mmol) was added followed by DIPEA (0.38 g, 2.94 mmol). The mixture was stirred for 4 hours then added dropwise into 5% aqueous sodium bicarbonate (100 mL) and ice to give a brown solid which was filtered and dried. A small part was purified after it was suspended in a mixture of acetonitrile and 1N HCl, filtered and washed (water) and dried to afford pure 6-bromo-8-trifluoromethyl-imidazo[1,2-a]pyridine-2-carboxylic acid (thiophen-2-ylmethyl)-amide. The rest was used for the next step without further purification. ¹H NMR (d₆-DMSO, 300 MHz) δ 9.18 (s, 1H), 8.85 (t, 1H, J=6 Hz), 8.45 (s, 1H), 7.96 (s, 1H), 7.36 (d, 1H, J=1.5 Hz), 7.00 (d, 1H, J=3.3 Hz), 6.93 (t, 1H, J=6 Hz), 4.62 (d, 2H, J=6 Hz); MS (ESI) m/z=405.9 (MH⁺).

### Example 37

### 6-Phenyl-8-trifluoromethyl-imidazo[1,2-a]pyridine-2-carboxylic Acid (thiophen-2-ylmethyl)-amide (Compound 137)

8-Trifluoromethyl-imidazo[1,2-a]pyridine-2-carboxylic acid (thiophen-2-ylmethyl)-amide (0.098 gm, 0.24 mmol) and phenyl boronic acid were dissolved in 1,4-dioxane (3 mL) and saturated aqueous sodium bicarbonate (1 mL) was added. Argon was bubbled through this mixture for 1 minute, then tetrkis(triphenylphosphine)palladium(0) (0.014 g, 0.012 mmol) was added and the mixture refluxed for 4 hours. The mixture was partitioned between ethyl acetate and water and the organic layer was dried (MgSO₄) to afford the crude product. The product was purified by passing through a short silica column to afford 6-phenyl-8-trifluoromethyl-imidazo[1,2-a]pyridine-2-carboxylic acid (thiophen-2-ylmethyl)-amide (0.058 gm, 60%) as a white solid. ¹H NMR (d₆-DMSO, 300 MHz) δ 9.20 (s, 1H), 8.82 (t, 1H, J=6 Hz), 8.52 (s, 1H), 8.08 (s, 1H), 7.76 (d, 2H, J=7.8 Hz), 7.43 (m, 3H), 7.35 (d, 1H, J=3.6 Hz), 7.01 (d, 1H, J=2.4 Hz), 6.94 (dd, 1H, J=3.6, 5.4 Hz), 4.64 (d, 2H, J=6.3 Hz); MS (ESI) m/z=402.1 (MH⁺).

### Example 38

### 6-Furan-2-yl-8-trifluoromethyl-imidazo[1,2-a]pyridine-2-carboxylic Acid (thiophen-2-ylmethyl)-amide (Compound 138)

Prepared using similar procedure as for compound 137; MS (ESI) m/z=392.0 (MH⁺).

### Example 39

### 3-Bromo-6-phenyl-8-trifluoromethyl-imidazo[1,2-a]pyridine-2-carboxylic Acid (thiophen-2-ylmethyl)-amide (Compound 139)

6-Phenyl-8-trifluoromethyl-imidazo[1,2-a]pyridine-2-carboxylic acid (thiophen-2-ylmethyl)-amide (0.045 g, 0.11 mmol) was dissolved in DMF (3 mL), NBS (0.02 g, 0.11 mmol) was added and the mixture stirred for 2 hours. The mixture was concentrated to 1 mL and added dropwise into ice-water (50 mL). The crude product crashed out and was purified using a silica column to afford 3-bromo-6-phenyl-8-trifluoromethyl-imidazo[1,2-a]pyridine-2-carboxylic acid (thiophen-2-ylmethyl)-amide (0.05 g, 95%). ¹H NMR (d₆-DMSO, 300 MHz) [symbol] 8.88 (t, 1H, J=6.3 Hz), 8.70 (s, 1H), 8.18 (s, 1H), 7.83 (d, 2H, J=7.2 Hz), 7.49 (m, 3H), 7.37 (d, 1H, J=4.5 Hz), 7.03 (d, 1H, J=3.3 Hz), 6.95 (dd, 1H, J=3.6, 4.8 Hz), 4.63 (d, 2H, J=6.0 Hz); MS (ESI) m/z=481.7 (MH⁺).

### Example 40

### 6-(4-Morpholin-4-yl-phenyl)-8-trifluoromethyl-imidazo[1,2-a]pyridine-2-carboxylic Acid (thiophen-2-ylmethyl)-amide (Compound 140)

Prepared using similar procedure as for compound 137.

¹H NMR (d₆-DMSO, 300 MHz) [symbol] 9.10 (s, 1H), 8.80 (t, 1H, J=5.4 Hz), 8.48 (s, 1H), 8.03 (s, 1H), 7.63 (d, 2H, J=8.4 Hz), 7.36 (dd, 1H, J=1.2, 5.1 Hz), 7.06 (d, 2H, J=9.3 Hz), 7.01 (d, 1H, J=3.6 Hz), 6.92 (dd, 1H, J=3.6, 4.8 Hz), 4.63 (d, 2H, J=6.6 Hz), 3.75 (br t, 4H), 3.18 (br t, 4H); MS (ESI) m/z=487.1 (MH⁺).

### Example 41

### 6-(5-Methyl-pyridin-3-yl)-8-trifluoromethyl-imidazo[1,2-a]pyridine-2-carboxylic Acid (thiophen-2-ylmethyl)-amide (Compound 141)

Prepared using similar procedure as for compound 137.

¹H NMR (d₆-DMSO, 300 MHz) δ 9.28 (s, 1H), 8.85 (t, 1H, J=6.6 Hz), 8.78 (br s, 1H), 8.51 (s, 1H), 8.47 (s, 1H), 8.17 (s, 1H), 8.05 (s, 1H), 7.36 (dd, 1H, J=1.5, 5.4 Hz), 7.01 (d, 1H, J=3.3 Hz), 6.94 (dd, 1H, J=3.6, 5.1 Hz), 4.64 (d, 2H, J=6.3 Hz), 2.39 (s, 3H); MS (ESI) m/z=417.1 (MH⁺).

### Example 42

### 6-(3-Morpholin-4-yl-phenyl)-8-trifluoromethyl-imidazo[1,2-a]pyridine-2-carboxylic Acid (thiophen-2-ylmethyl)-amide (Compound 142)

Prepared using similar procedure as for compound 137.

¹H NMR (d₆-DMSO, 300 MHz) [symbol] 9.17 (s, 1H) 8.82 (t, 1H, J=6.3 Hz), 8.49 (s, 1H), 8.07 (s, 1H), 7.35 (m, 2H), 7.27 (br s, 1H), 7.16 (br d, 1H), 7.01 (m, 2H), 6.94 (dd, 1H, J=3.6, 5.4 Hz), 4.64 (d, 2H, J=6.3 Hz), 3.76 (br t, 4H), 3.21 (br t, 4H); MS (ESI) m/z=487.1 (MH⁺).

### Example 43

### 7-Trifluoromethyl-pyrazolo[1,5-a]pyridine-2-carboxylic Acid (thiophen-2-ylmethyl)-amide (Compound 143)

Prepared using a similar procedure as for compound 144 with 2-trifluoromethylpyridine as starting material.

¹H NMR (d₆-DMSO, 300 MHz) δ 4.65 (d, 2H, J=5.9 Hz), 6.95 (dd, 1H, J=3.5, 5 Hz), 7.02 (dd, 1H, J=1.2, 3.5 Hz), 7.26 (s, 1H), 7.38 (dd, 1H, J=1.2, 5 Hz), 7.41 (dd, 1H, J=7, 9 Hz), 7.66 (d, 1H, J=6.2 Hz), 8.11 (d, 1H, J=8.5 Hz), 8.99 (t, 1H, J=5.9 Hz); MS (ESI) m/z=326.0 (MH⁺).

### Example 44

### 7-Chloro-5-phenyl-pyrazolo[1,5-a]pyridine-2-carboxylic Acid (thiophen-2-ylmethyl)-amide (Compound 144)

### Step 1: 1-Amino-4-bromo-2-chloro-pyridinium Mesitylenesulfonate

To a stirred solution of 4-bromo-2-chloropyridine (2.048 g, 10.64 mmol) in CH₂Cl₂ (5 mL) was added O-mesitylsulfonylhydroxylamine (MSH, 2.52 g, 11.71 mmol). After 7 hours, the solvent was concentrated and triturated with Et₂O to give a white syrup. The solvent was decanted and triturated again with Et₂O. The product was dried under vacuum to give 1-amino-4-bromo-2-chloro-pyridinium mesitylenesulfonate (3.16 g, 73%). ¹H NMR (d₆-DMSO, 300 MHz) δ 2.17 (s, 6H), 2.49 (s, 12H), 6.73 (s, 4H), 8.23 (dd, 1H, J=2.3, & Hz), 8.41 (brs, 2H), 8.75 (d, 1H, J=7 Hz), 8.77 (d, 1H, J=2.3 Hz); MS (ESI) m/z=206.9, 208.9 (MNa⁺).

### Step 2: 5-Bromo-7-chloro-pyrazolo[1,5-a]pyridine-2,3-dicarboxylic Acid Dimethyl Ester

To a solution of 1-amino-4-bromo-2-chloro-pyridinium mesitylenesulfonate (3.16 g, 7.75 mmol) in DMF (15 mL) was added K₂CO₃ (3.21 g, 23.25 mmol) followed by dropwise addition of dimethyl acetylenedicarboxylate (1.43 mL, 11.63 mmol). Air was then bubbled through the mixture. After 3 hours, the precipitate was filtered and the solvent was concentrated under reduced pressure. The crude material was diluted with EtOAc (200 mL) and washed successively with aqueous HCl (2N, 50 mL), saturated aqueous NaHCO₃ (2×50 mL), then brine (50 mL). The organic extracts were dried (MgSO₄), filtered and concentrated. Column chromatography [n-hex/EtOAc (5:1 v/v) to n-hex/EtOAc (3.5:1 v/v)] of the crude brown solid gave 5-bromo-7-chloro-pyrazolo[1,5-a]pyridine-2,3-dicarboxylic acid dimethyl ester (0.85 g, 23%). ¹H NMR (d₆-DMSO, 300 MHz) δ 3.86 (s, 3H), 3.93 (s, 3H), 7.93 (d, 1H, J=1.8 Hz), 8.26 (d, 1H, J=1.8 Hz); MS (ESI) m/z=346.9 (MH⁺).

### Step 3: 5-Bromo-7-chloro-pyrazolo[1,5-a]pyridine-2-carboxylic Acid

A suspension of 5-bromo-7-chloro-pyrazolo[1,5-a]pyridine-2,3-dicarboxylic acid dimethyl ester (720 mg, 2.07 mmol) was heated at 90° C. in 50% v/v sulfuric acid for 29 hours. The mixture was cooled with an ice-water bath followed by addition of NaOH solution (50% w/v, ˜60 mL) and water to dissolve the product. The aqueous phase was then washed with Et₂O (2×70 mL). The aqueous phase was separated and acidified with 2N HCl and extracted with EtOAc (250 mL, 150 mL). The organic phase was dried (Na₂SO₄), filtered and concentrated to give 5-bromo-7-chloro-pyrazolo[1,5-a]pyridine-2-carboxylic acid (0.61 g, quantitative) as a beige solid. ¹H NMR (d₆-DMSO, 300 MHz) δ 7.17 (s, 1H), 7.65 (d, 1H, J=2 Hz), 8.17 (d, 1H, J=2 Hz), 13.39 (brs, 1H); MS (ESI) m/z=274.9, 276.9 (MH⁺).

### Step 4: 5-Bromo-7-chloro-pyrazolo[1,5-a]pyridine-2-carboxylic Acid (thiophen-2-ylmethyl)-amide

A mixture of 5-bromo-7-chloro-pyrazolo[1,5-a]pyridine-2-carboxylic acid (0.61 g, 2.21 mmol), 2-thiophenemethylamine (0.25 mL, 2.44 mmol), N,N-di-isopropylethylamine (1.16 mL, 6.64 mmol), and PyBroP® (1.135 g, 2.44 mmol) was stirred in DMF (10 mL) at room temperature. After 15 min, the mixture was diluted with EtOAc (150 mL) and washed successively with 2N HCl (2×30 mL), saturated aqueous NaHCO₃ (30 mL), and brine (30 mL). The organic phase was filtered through a small pad of silica gel and concentrated to give 5-bromo-7-chloro-pyrazolo[1,5-a]pyridine-2-carboxylic acid (thiophen-2-ylmethyl)-amide (983.6 mg, quantitative) as a foam. ¹H NMR (d₆-DMSO, 300 MHz) δ 4.63 (d, 2H, J=5.9 Hz), 6.95 (dd, 1H, J=3.2, 5 Hz), 7.02 (dd, 1H, J=0.9, 3.2 Hz), 7.14 (s, 1H), 7.37 (dd, 1H, J=1.5, 5 Hz), 7.63 (d, 1H, J=2 Hz), 8.17 (d, 1H, J=2 Hz), 9.12 (t, 1H, J=5.9 Hz); MS (ESI) m/z=369.9, 371.9 (MH⁺).

### Step 5: 7-Chloro-5-phenyl-pyrazolo[1,5-a]pyridine-2-carboxylic Acid (thiophen-2-ylmethyl)-amide (Compound 144)

A mixture of 5-bromo-7-chloro-pyrazolo[1,5-a]pyridine-2-carboxylic acid (thiophen-2-ylmethyl)-amide (890 mg, 2.40 mmol), phenylboronic acid (439 mg, 3.60 mmol) and Pd(PPh₃)₄ (139 mg, 0.12 mmol) was heated in aq. K₃PO₄ (1M, 4 mL) and 1,4-dioxane (12 mL) at 80° C. for 10 min under microwave conditions. Dioxane was removed under reduced pressure and the mixture was diluted with EtOAc (100 mL). The aqueous phase was separated and the organic phase was washed with saturated aqueous NaHCO₃ (2×30 mL), then brine (30 mL). The organic phase was dried (Na₂SO₄), filtered and concentrated. Column chromatography [n-hex/EtOAc (4:1 v/v) to n-hex/EtOAc (2.5:1 v/v)] of the crude material gave 7-chloro-5-phenyl-pyrazolo[1,5-a]pyridine-2-carboxylic acid (thiophen-2-ylmethyl)-amide (748.5 mg, 85%) as a white solid. ¹H NMR (d₆-DMSO, 300 MHz) δ 4.65 (d, 2H, J=6.2 Hz), 6.96 (dd, 1H, J=3.2, 5 Hz), 7.03 (dd, 1H, J=1.2, 3.2 Hz), 7.22 (s, 1H), 7.38 (dd, 1H, J=1.2, 5 Hz), 7.41-7.54 (m, 3H), 7.76 (d, 2H, J=2 Hz), 7.83-7.87 (m, 2H), 8.17 (d, 1H, J=2 Hz), 9.09 (t, 1H, J=6.2 Hz); MS (ESI) m/z=368.0 (MH⁺).

### Example 45

### 7-Chloro-5-furan-2-yl-pyrazolo[1,5-a]pyridine-2-carboxylic Acid (thiophen-2-ylmethyl)-amide (Compound 145)

A mixture of 5-bromo-7-chloro-pyrazolo[1,5-a]pyridine-2-carboxylic acid (thiophen-2-ylmethyl)-amide (40.9 mg, 0.11 mmol), 2-furanboronic acid (16.1 mg, 0.14 mmol) and Pd(PPh₃)₄ (6.4 mg, 0.0055 mmol) was heated in aq. K₃PO₄ (1M, 0.2 mL) and 1,4-dioxane (0.6 mL) at 60° C. for 20 min under microwave conditions. The mixture was diluted with EtOAc (10 mL) and washed successively with water (5 mL), saturated aqueous NaHCO₃ (5 mL), and brine (5 mL). The organic phase was dried (Na₂SO₄), filtered and concentrated. The product was purified by preparative HPLC (40-100% ACN gradient) and then silica gel column [CH₂Cl₂/ACN (95:5 v/v)] to give 7-chloro-5-furan-2-yl-pyrazolo[1,5-a]pyridine-2-carboxylic acid (thiophen-2-ylmethyl)-amide (7.7 mg, 20%) as a white powder. ¹H NMR (d₆-DMSO, 300 MHz) δ 4.64 (d, 2H, J=5.9 Hz), 6.68 (dd, 1H, J=1.7, 3.2 Hz), 6.95 (dd, 1H, J=3.2, 5 Hz), 7.02 (dd, 1H, J=1.2, 3.5 Hz), 7.21 (s, 1H), 7.28 (d, 1H, J=3.2 Hz), 7.37 (dd, 1H, J=1.2, 5 Hz), 7.75 (d, 1H, J=1.8 Hz), 7.87 (d, 1H, J=1.2 Hz), 8.03 (d, 1H, J=1.8 Hz), 9.07 (t, 1H, J=5.9 Hz); MS (ESI) m/z=358 (MH⁺).

### Example 46

### 6-Furan-2-yl-8-trifluoromethylimidazo[1,2-a]pyridine-2-carboxylic Acid Methyl-thiophen-2-ylmethyl-amide (Compound 146)

6-Furan-2-yl-8-trifluoromethyl-imidazo[1,2-a]pyridine-2-carboxylic acid (thiophen-2-ylmethyl)-amide (0.1 gm, 0.26 mmol) was dissolved in DMF (0.5 mL) and added dropwise to a suspension of NaH (60%, 0.012 gm, 0.31 mmol) in DMF (2 mL). The mixture was stirred for min. Methyl iodide (0.019 mL, 0.31 mmol) was added and the mixture stirred at room temperature over 12 hours. The reaction was quenched with water and the product was extracted with ethyl acetate. The crude product was purified through silica gel chromatography to afford 6-furan-2-yl-8-trifluoromethylimidazo[1,2a]pyridine-2-6 acid methyl-thiophen-2-ylmethyl-amide (0.02 g, 20%). ¹H NMR (d₆-DMSO, 300 MHz) [symbol] 9.18 (s, 0.5H), 9.15 (s, 0.5H), 8.56 (s, 0.5H), 8.54 (s, 0.5H), 8.14 (br s, 1H), 7.84 (br s, 1H), 7.42 (m, 1H), 7.23 (d, 1H, J=3.3 Hz), 7.09 (m, 1H), 6.95 (m, 1H), 6.66 (m, 1H), 5.48 (s, 1H), 4.80 (s, 1H), 3.39 (s, 1.5H), 2.96 (s, 1.5H); MS (ESI) m/z=406.0 (MH⁺).

### Example 47

### 5-phenyl-7-trifluoromethyl-pyrazolo[1,5-a]pyridine-2-carboxylic Acid methyl-thiophen-2-ylmethyl-amide (Compound 147)

Prepared using a similar procedure as for compound 137.

¹H NMR (d₆-DMSO, 300 MHz) δ 3.00 (s, 3.6H), 3.23 (s, 3H), 4.85 (s, 2.4H), 5.19 (s, 2H), 6.95-7.05 (m, 3.5H), 7.12-7.18 (m, 3.5H), 7.42-7.57 (m, 9H), 7.84-7.92 (m, 6H), 8.41 (dd, 2H, J=1.5, 6.3 Hz)); MS (ESI) m/z=416.1 (MH⁺).

### Example 48

### 7-Morpholin-4-yl-5-phenyl-pyrazolo[1,5-a]pyridine-2-carboxylic Acid (thiophen-2-ylmethyl)-amide (Compound 148)

7-Chloro-5-phenyl-pyrazolo[1,5-a]pyridine-2-carboxylic acid (thiophen-2-ylmethyl)-amide (45 mg, 0.12 mmol) was treated with excess morpholine and heated in DMF under microwave conditions to give 7-morpholin-4-yl-5-phenyl-pyrazolo[1,5-a]pyridine-2-carboxylic acid (thiophen-2-ylmethyl)-amide (28.3 mg, 55%) as a white powder after column chromatography. ¹H NMR (d₆-DMSO, 300 MHz) δ 3.46-3.48 (brs, 4H), 3.86-3.90 (m, 4H), 4.67 (d, 2H, J=6.2 Hz), 6.67 (d, 1H, J=1.8 Hz), 6.96 (dd, 1H, J=3.2, 5 Hz), 7.03 (dd, 1H, J=1.2, 3.5 Hz), 7.03 (s, 1H), 7.38 (dd, 1H, J=1.2, 5 Hz), 7.38-7.52 (m, 3H), 7.72 (d, 1H, J=1.8 Hz), 7.78-7.83 (m, 2H), 8.98 (t, 1H, J=6.2 Hz); MS (ESI) m/z=419.1 (MH⁺).

### Example 49

### 7-(2-Morpholin-4-yl-ethylamino)-5-phenyl-pyrazolo[1,5-a]pyridine-2-carboxylic Acid (thiophen-2-ylmethyl)-amide (Compound 149) and

### 7-dimethylamino-5-phenyl-pyrazolo[1,5-a]pyridine-2-carboxylic Acid (thiophen-2-ylmethyl)-amide (Compound 150)

7-Chloro-5-phenyl-pyrazolo[1,5-a]pyridine-2-carboxylic acid (thiophen-2-ylmethyl)-amide (50 mg) was treated with excess 4-(2-aminoethyl)morpholine and heated in DMF under microwave conditions to give 7-(2-morpholin-4-yl-ethylamino)-5-phenyl-pyrazolo[1,5-a]pyridine-2-carboxylic acid (thiophen-2-ylmethyl)-amide (6.1 mg) and 7-dimethylamino-5-phenyl-pyrazolo[1,5-a]pyridine-2-carboxylic acid (thiophen-2-ylmethyl)-amide (5.6 mg) after HPLC purification.

Data for 7-(2-morpholin-4-yl-ethylamino)-5-phenyl-pyrazolo[1,5-a]pyridine-2-carboxylic acid (thiophen-2-ylmethyl)-amide: ¹H NMR (d₆-DMSO, 300 MHz) δ 3.31-3.28 (m, 2H), 3.5-4.08 (m, 10H), 4.70 (d, 2H, J=5.9 Hz), 6.50 (d, 1H, J=1.8 Hz), 6.97 (dd, 1H, J=3.5, Hz), 6.98 (s, 1H), 7.05 (dd, 1H, J=1.5, 3.5 Hz), 7.34 (brs, 1H), 7.37-7.52 (m, 3H), 7.41 (dd, 1H, J=1.8 Hz), 7.81-7.84 (m, 2H), 8.83 (t, 1H, J=5.9 Hz), 10.18 (s, 1H); MS (ESI) m/z=462.1 (MH⁺).

Data for 7-dimethylamino-5-phenyl-pyrazolo[1,5-a]pyridine-2-carboxylic acid (thiophen-2-ylmethyl)-amide: ¹H NMR (d₆-DMSO, 300 MHz) δ 3.13 (s, 6H), 4.65 (d, 2H, J=6 Hz), 6.60 (d, 1H, J=1.8 Hz), 6.95 (dd, 1H, J=3.2, 5 Hz), 7.00 (s, 1H), 7.03 (dd, 1H, J=1.2, 3.2 Hz), 7.38 (dd, 1H, J=1.2, 5 Hz), 7.40-7.52 (m, 3H), 7.64 (d, 1H, J=1.8 Hz), 7.77-7.82 (m, 2H), 9.02 (t, 1H, J=6 Hz); MS (ESI) m/z=377.1 (MH⁺).

### Example 51

### 6-Bromo-3-chloro-8-trifluoromethyl-imidazo[1,2-a]pyridine-2-carboxylic Acid (thiophen-2-ylmethyl)-amide (Compound 151)

### Step 1: 6-Bromo-3-chloro-8-trifluoromethyl-imidazo[1,2-a]pyridine-2-carboxylic Acid Ethyl Ester

A mixture of 6-bromo-8-trifluoromethyl-imidazo[1,2-a]pyridine-2-carboxylic acid ethyl ester (Compound 134) (8.08 g, 23.97 mmol) and NCS (3.68 g, 27.56 mmol) was stirred in DMF (80 mL) at room temperature for 14.5 hours. The solvent was removed under reduced pressure to −20 mL and diluted with EtOAc (400 mL). The organic layer was washed successively with aqueous sodium thiosulfate (1M, 2×100 mL), saturated aqueous NaHCO₃ (100 mL) and brine (100 mL), filtered through a small pad of silica gel and concentrated to give 6-bromo-3-chloro-8-trifluoromethyl-imidazo[1,2-a]pyridine-2-carboxylic acid ethyl ester as a yellow solid (7.64 g, 86%). ¹H NMR (d₆-DMSO, 300 MHz) δ 1.35 (t, 3H, J=7 Hz), 4.37 (q, 2H, J=7 Hz), 8.11 (brs, 1H), 9.01 (brs, 1H); MS (ESI) m/z=370.9, 372.9, 374.9 (MH⁺).

### Step 2: 6-Bromo-3-chloro-8-trifluoromethyl-imidazo[1,2-a]pyridine-2-carboxylic Acid

A mixture of 6-bromo-3-chloro-8-trifluoromethyl-imidazo[1,2-a]pyridine-2-carboxylic acid ethyl ester (0.8 g, 2.15 mmol) in acetonitrile (ACN, 4 mL) and 6N HCl (8 mL) was heated at 140° C. for 15 min under microwave conditions. The reaction was repeated four times and the precipitate filtered and discarded. The filtrate was concentrated to −10 mL and triturated with water (70 mL). The precipitate was filtered and dried under high vacuum to give 6-bromo-3-chloro-8-trifluoromethyl-imidazo[1,2-a]pyridine-2-carboxylic acid (2.23 g, 73%) as a beige solid. ¹H NMR (d₆-DMSO, 300 MHz) δ 8.09 (brs, 1H), 8.98 (d, 1H, J=0.8 Hz), 13.5 (brs, 1H); MS (ESI) m/z=342.9, 344.9, 346.9 (MH⁺).

### Step 3: 6-Bromo-3-chloro-8-trifluoromethyl-imidazo[1,2-a]pyridine-2-carboxylic Acid (thiophen-2-ylmethyl)-amide (Compound 151)

A solution of 6-bromo-3-chloro-8-trifluoromethyl-imidazo[1,2-a]pyridine-2-carboxylic acid (3.29 g, 9.58 mmol), 2-thiophenemethylamine (1.13 mL, 11.01 mmol), N,N-di-isopropylethylamine (6.67 mL, 38.31 mmol), and PyBroP® (5.50 g, 11.01 mmol) was stirred in DMF (20 mL) at room temperature for 25 min. The mixture was diluted with EtOAc (500 mL) and washed successively with 2N HCl (2×75 mL), saturated aqueous NaHCO₃ (2×75 mL), and brine (75 mL). The organic phase was dried (Na₂SO₄), filtered and concentrated. Crystallization of the crude material from EtOAc/n-hex gave 6-bromo-3-chloro-8-trifluoromethyl-imidazo[1,2-a]pyridine-2-carboxylic acid (thiophen-2-ylmethyl)-amide (3.29 g, 78%) as white crystals. ¹H NMR (d₆-DMSO, 300 MHz) δ 4.62 (d, 2H, J=6.2 Hz), 6.94 (dd, 1H, J=3.5, 5 Hz), 7.02 (dd, 1H, J=1.2, 3.2 Hz), 7.37 (dd, 1H, J=1.2, 5 Hz), 8.09 (m, 1H), 8.93 (t, 1H, J=6.2 Hz), 8.98 (brs, 1H); MS (ESI) m/z=437.9, 439.9 (MH⁺).

### Example 52

### 3-Chloro-6-furan-2-yl-8-trifluoromethyl-imidazo[1,2-a]pyridine-2-carboxylic Acid (thiophen-2-ylmethyl)-amide (Compound 152)

Prepared using similar procedure as for compound 157.

¹H NMR (d₆-DMSO, 300 MHz) δ 4.65 (d, 2H), 6.69 (m, 1H), 6.96 (m, 2H), 7.36 (m, 2H), 7.87 (d, 1H), 8.25 (s, 1H), 8.68 (s, 1H), 8.90 (t, 1H); MS (ESI) m/z=426.7 (M+).

### Example 53

### 7-Methylamino-5-phenyl-pyrazolo[1,5-a]pyridine-2-carboxylic Acid (thiophen-2-ylmethyl)-amide (Compound 153)

7-Chloro-5-phenyl-pyrazolo[1,5-a]pyridine-2-carboxylic Acid (thiophen-2-ylmethyl)-amide (50 mg) was treated with methylamine (2M in THF) and heated at 120° C. to give 7-methylamino-5-phenyl-pyrazolo[1,5-a]pyridine-2-carboxylic acid (thiophen-2-ylmethyl)-amide (34.2 mg) after column chromatography. ¹H NMR (d₆-DMSO, 300 MHz) δ 3.06 (d, 3H, J=5 Hz), 4.69 (d, 2H, J=6.2 Hz), 6.27 (d, 1H, J=1.8 Hz), 6.91 (s, 1H), 6.97 (dd, 1H, J=3.2, 5 Hz), 6.99 (q, 1H, J=5 Hz), 7.05 (dd, 1H, J=1.2, 3.5 Hz), 7.28 (d, 1H, J=1.8 Hz), 7.41 (dd, 1H, J=1.2, 5 Hz), 7.36-7.51 (m, 3H), 7.76-7.80 (m, 2H), 8.81 (t, 1H, J=6.2 Hz); MS (ESI) m/z=363.1 (MH⁺).

### Example 54

### 7-(2-Hydroxy-ethylamino)-5-phenyl-pyrazolo[1,5-a]pyridine-2-carboxylic Acid (thiophen-2-ylmethyl)-amide (Compound 154)

7-Chloro-5-phenyl-pyrazolo[1,5-a]pyridine-2-carboxylic acid (thiophen-2-ylmethyl)-amide (50 mg) was treated with excess ethanolamine and heated in iso-amyl alcohol at 135° C. Purification by reversed phase HPLC gave 7-(2-hydroxy-ethylamino)-5-phenyl-pyrazolo[1,5-a]pyridine-2-carboxylic acid (thiophen-2-ylmethyl)-amide (12.1 mg). ¹H NMR (d₆-DMSO, 300 MHz) δ 3.53 (q, 2H, J=5.6 Hz), 3.71 (q, 2H, J=5.3 Hz), 4.67 (d, 2H, J=5.9 Hz), 4.97 (t, 1H, J=5.3 Hz), 6.40 (d, 1H, J=1.8 Hz), 6.79 (t, 1H, J=5.9 Hz), 6.92 (s, 1H), 6.96 (dd, 1H, J=3.5, 5 Hz), 7.04 (dd, 1H, J=1.2, 3.2 Hz), 7.29 (d, 1H, J=1.8 Hz), 7.39 (dd, 1H, J=1.2, 5 Hz), 7.37-7.51 (m, 3H), 7.78-7.80 (m, 2H), 9.01 (t, 1H, J=5.9 Hz); MS (ESI) m/z=393.1 (MH⁺).

### Example 55

### 6,8-Bis-trifluoromethyl-imidazo[1,2-a]pyridine-2-carboxylic Acid (thiophen-2-ylmethyl)-amide (Compound 155)

Using similar procedure as for the preparation of compound 136, 3,5-bis(trifluoromethyl)-2-aminopyridine was used as starting material to give 6,8-bis-trifluoromethyl-imidazo[1,2a]pyridine-2-carboxylic acid (thiophen-2-ylmethyl)-amide. ¹H NMR (d₆-DMSO, 300 MHz) [symbol] 9.52 (s, 1H), 8.95 (t, 1H, J=6.3 Hz), 8.60 (s, 1H), 8.07 (s, 1H), 7.35 (dd, 1H, J=1.2, 4.8 Hz), 7.01 (dd, 1H, J=0.9, 3.3 Hz), 6.93 (dd, 1H, J=3.3, 4.8 Hz), 4.63 (d, 2H, J=6.3 Hz); MS 394.0 (MH⁺).

### Example 56

### 6-Furan-2-yl-3-methyl-8-trifluoromethyl-imidazo[1,2-a]pyridine-2-carboxylic Acid (thiophen-2-ylmethyl)-amide (Compound 156)

### Step 1: 2-Bromo-2-oxo-butyric Acid

Bromine (3.65 g, 22.8 mmol) was added dropwise to 2-oxo-butyric acid (2.33 g, 22.8 mmol). A vigorous reaction resulted. The mixture was stirred for 30 min, then water and ethyl acetate were added and the organic layer separated. This was washed with 5% NaHSO₃, water, then brine. The organic extracts were concentrated under reduced pressure to afford 3-bromo-2-oxo-butyric acid (2.3 g, 56%).

### Step 2: 6-Furan-2-yl-3-methyl-8-trifluoromethyl-imidazo[1,2-a]pyridine-2-carboxylic Acid (thiophen-2-ylmethyl)-amide (Compound 156)

Using similar procedure as for the preparation of compound but with the use of 2-bromo-2-oxo-butyric acid gave 6-furan-2-yl-3-methyl-8-trifluoromethyl-imidazo[1,2-a]pyridine-2-carboxylic acid (thiophen-2-ylmethyl)-amide. ¹H NMR (d₆-DMSO, 300 MHz) [symbol] 8.74 (s, 1H), 8.69 (t, 1H, J=6.6 Hz), 8.11 (s, 1H), 7.85 (s, 1H), 7.35 (br d, 1H), 7.27 (d, 1H, J=3.6 Hz), 7.00 (br s, 1H), 6.93 (m, 1H), 6.67 (dd, 1H, J=1.8, 3.3 Hz), 4.63 (d, 2H, J=6.0 Hz), 2.88 (s, 3H); MS (ESI) m/z=406.1 (MH⁺).

### Example 57

### 3-Chloro-6-furan-3-yl-8-trifluoromethyl-imidazo[1,2-a]pyridine-2-carboxylic Acid (thiophen-2-ylmethyl)-amide (compound 157)

A mixture of 6-bromo-3-chloro-8-trifluoromethyl-imidazo[1,2-a]pyridine-2-carboxylic acid (thiophen-2-ylmethyl)-amide (43.9 mg, 0.1 mmol), 3-furanboronic acid (16.8 mg, 0.15 mmol) and Pd(PPh₃)₄ (5.8 mg, 0.005 mmol) in aqueous K₃PO₄ (1M, 0.3 mL) and 1,4-dioxane (0.9 mL) was heated at 100° C. for 3 min under microwave conditions. The mixture was diluted with EtOAc (40 mL) and washed with saturated aqueous NaHCO₃ (20 mL), then brine (20 mL). The organic phase was dried (Na₂SO₄), filtered and concentrated. Purification of the crude product by preparative HPLC (30-100% ACN gradient) followed by crystallization from EtOAc/n-hex gave 3-chloro-6-furan-3-yl-8-trifluoromethyl-imidazo[1,2-a]pyridine-2-carboxylic acid (thiophen-2-ylmethyl)-amide (15.7 mg, 37%) as off-white solid. ¹H NMR (d₆-DMSO, 300 MHz) δ 4.64 (d, 2H, J=6.4 Hz), 6.95 (dd, 1H, J=3.5, 5 Hz), 7.03 (dd, 1H, J=1.2, 3.5 Hz), 7.32 (dd, 1H, J=0.9, 1.8 Hz), 7.37 (dd, 1H, J=1.2, 5 Hz), 7.83 (t, 1H, J=1.8 Hz), 8.22 (s, 1H), 8.55 (s, 1H), 8.81 (s, 1H), 8.88 (t, 1H, J=6.4 Hz); MS (ESI) m/z=426 (MH⁺).

### Example 58

### 3-Chloro-6-furan-2-yl-8-trifluoromethyl-imidazo[1,2-a]pyridine-2-carboxylic Acid (furan-2-ylmethyl)-amide (Compound 158)

### Step 1: 3-Chloro-6-furan-2-yl-8-methyl-imidazo[1,2-a]pyridine-2-carboxylic Acid Ethyl ester

A mixture of 6-bromo-3-chloro-8-trifluoromethyl-imidazo[1,2-a]pyridine-2-carboxylic acid ethyl ester (1.2 g, 3.23 mmol) and 2-furanboronic acid (722.8 mg, 6.45 mmol) in aqueous K₃PO₄ (1M, 4 mL) and 1,4-dioxane (12 mL) was heated at 140° C. for 15 min under microwave conditions. The reaction was repeated 4 times and combined. Upon cooling, the precipitate was filtered and rinsed with EtOAc to give 3-chloro-6-furan-2-yl-8-trifluoromethyl-imidazo[1,2-a]pyridine-2-carboxylic acid ethyl ester (5.42 g, 94%) as a beige solid. ¹H NMR (d₆-DMSO, 300 MHz) δ 1.36 (t, 3H, J=7 Hz), 4.38 (q, 2H, J=7 Hz), 6.70 (dd, 1H, J=1.8, 3.5 Hz), 7.41 (d, 1H, J=3.2 Hz), 7.88 (dd, 1H, J=0.6, 1.8 Hz), 8.27 (m, 1H), 8.69 (s, 1H); MS (ESI) m/z=359, 361 (MH⁺).

### Step 2: 3-Chloro-6-furan-2-yl-8-trifluoromethyl-imidazo[1,2-a]pyridine-2-carboxylic Acid

A mixture of 3-chloro-6-furan-2-yl-8-trifluoromethyl-imidazo[1,2-a]pyridine-2-carboxylic acid ethyl ester (0.5 g, 1.39 mmol) in 1,4-dioxane (5 mL) and 6N HCl (10 mL) was heated at 120° C. for 45 min under microwave conditions. Upon cooling, the solvent was removed under reduced pressure to give 3-chloro-6-furan-2-yl-8-trifluoromethyl-imidazo[1,2-a]pyridine-2-carboxylic acid (536 mg) as a yellow solid which was used for the next step without further purification. ¹H NMR (d₆-DMSO, 300 MHz) δ 6.70 (dd, 1H, J=1.8, 3.5 Hz), 7.40 (d, 1H, J=3.5 Hz), 7.88 (d, 1H, J=1.8 Hz), 8.25 (s, 1H), 8.68 (s, 1H); MS (ESI) m/z=331, 333 (MH⁺).

### Step 3: 3-Chloro-6-furan-2-yl-8-trifluoromethyl-imidazo[1,2-a]pyridine-2-carboxylic Acid (furan-2-ylmethyl)-amide (Compound 158)

A mixture of 3-chloro-6-furan-2-yl-8-trifluoromethyl-imidazo[1,2-a]pyridine-2-carboxylic acid (50 mg, 0.151 mmol), 2-furylmethylamine (16 μL, 0.182 mmol), N,N-di-isopropylethylamine (105.4 μL, 0.605 mmol), and HATU (69 mg, 0.182 mmol) was stirred in DMF (0.8 mL) at room temperature for 30 min. The mixture was diluted with EtOAc (20 mL) and washed successively with 2N HCl (2×10 mL), saturated aqueous NaHCO₃ (10 mL), and brine (10 mL). The organic phase was dried (Na₂SO₄), filtered and concentrated. Column chromatography of the crude material gave 3-chloro-6-furan-2-yl-8-trifluoromethyl-imidazo[1,2-a]pyridine-2-carboxylic acid (furan-2-ylmethyl)-amide (32.9 mg, 53%) as a white solid. ¹H NMR (d₆-DMSO, 300 MHz) δ 4.49 (d, 2H, J=6.2 Hz), 6.26 (brd, 1H, J=2.6 Hz), 6.39 (dd, 1H, J=1.8, 3.2 Hz), 6.70 (dd, 1H, J=1.8, 3.5 Hz), 7.40 (d, 1H, J=3.5 Hz), 7.56 (dd, 1H, J=0.9, 1.8 Hz), 7.88 (d, 1H, J=1.5 Hz), 8.26 (s, 1H), 8.70 (t, 1H, J=6.2 Hz), 8.70 (s, 1H); MS (ESI) m/z=410 (MH⁺).

### Example 59

### 3-Chloro-6-furan-2-yl-8-trifluoromethyl-imidazo[1,2-a]pyridine-2-carboxylic Acid (furan-3-ylmethyl)-amide (Compound 159)

Prepared using similar procedure as for compound 158.

¹H NMR (d₆-DMSO, 300 MHz) δ 4.31 (d, 2H, J=6.2 Hz), 6.48 (brs, 1H), 6.70 (dd, 1H, J=1.8, 3.5 Hz), 7.39 (d, 1H, J=3.5 Hz), 7.57 (d, 1H, J=1.4 Hz), 7.88 (d, 1H, J=1.8 Hz), 8.25 (s, 1H), 8.63 (t, 1H, J=6.2 Hz), 8.69 (s, 1H); MS (ESI) m/z=410 (MH⁺).

### Example 60

### 3-Chloro-6-thiophen-3-yl-8-trifluoromethyl-imidazo[1,2-a]pyridine-2-carboxylic Acid (thiophen-2-ylmethyl)-amide (compound 160)

Prepared using similar procedure as for compound 157.

¹H NMR (d₆-DMSO, 300 MHz) δ 4.63 (d, 2H, J=6.0 Hz), 6.95 (m, 1H), 7.02 (d, 1H, J=2.4 Hz), 7.37 (dd, 1H, J=1.2, 4.8 Hz), 7.74 (m, 1H), 7.83 (dd, 1H, J=1.2, 5.0 Hz), 8.29 (m, 1H), 8.87 (m, 2H); MS (ESI) m/z=442 (MH⁺).

### Example 61

### (3-Chloro-6-furan-2-yl-8-trifluoromethyl-imidazo[1,2-a]pyridin-2-yl)-(1,3-dihydro-isoindol-2-yl)-methanone (Compound 161)

Prepared using similar procedure as for compound 158.

¹H NMR (d₆-DMSO, 300 MHz) δ 4.92 (s, 2H), 5.20 (s, 2H), 6.71 (dd, 1H, J=1.8, 3.5 Hz), 7.28-7.44 (m, 3H), 7.89 (d, 1H, J=1.2 Hz), 8.27 (s, 1H), 8.73 (s, 1H); MS (ESI) m/z=432 (MH⁺).

### Example 62

### 3-Chloro-6-furan-2-yl-8-trifluoromethyl-imidazo[1,2-a]pyridine-2-carboxylic Acid (1-thiophen-2-yl-ethyl)-amide (Compound 162)

Prepared using similar procedure as for compound 158.

¹H NMR (d₆-DMSO, 300 MHz) δ 1.65 (d, 3H, J=7 Hz), 5.46 (pentet, 1H, J=7 Hz), 6.69 (dd, 1H, J=1.8, 3.5 Hz), 6.98 (dd, 1H, J=3.5, 5 Hz), 7.06 (dt, 1H, J=1.2, 3.5 Hz), 7.38-7.40 (m, 2H), 7.88 (dd, 1H, J=0.6, 1.8 Hz), 8.25 (s, 1H), 8.59 (d, 1H, J=8.8 Hz), 8.70 (s, 1H); MS (ESI) m/z=440 (MH⁺).

### Example 63

### 3-Chloro-6-furan-2-yl-8-trifluoromethyl-imidazo[1,2-a]pyridine-2-carboxylic Acid (pyridin-2-ylmethyl)-amide (Compound 163)

Prepared using similar procedure as for compound 158.

¹H NMR (d₆-DMSO, 300 MHz) δ 4.62 (d, 2H, J=5.9 Hz), 6.70 (dd, 1H, J=1.8, 3.5 Hz), 7.26 (ddd, 1H, J=0.9, 4.7, 7.3 Hz), 7.32 (brd, 1H, J=7.6 Hz), 7.40 (d, 1H, J=3.2 Hz), 7.75 (dt, 1H, J=2, 7.6 Hz), 7.98 (brd, 1H, J=1.2 Hz), 8.27 (s, 1H), 8.51 (ddd, 1H, J=0.9, 1.8, 4.7 Hz), 8.71 (s, 1H), 8.89 (t, 1H, J=5.9 Hz); MS (ESI) m/z=421 (MH⁺).

### Example 64

### 3-Chloro-6-furan-2-yl-8-trifluoromethyl-imidazo[1,2-a]pyridine-2-carboxylic Acid (pyridin-3-ylmethyl)-amide (Compound 164)

Prepared using similar procedure as for compound 158.

¹H NMR (d₆-DMSO, 300 MHz) δ 4.51 (d, 2H, J=6.2 Hz), 6.69 (dd, 1H, J=1.8, 3.5 Hz), 7.36 (ddd, 1H, J=0.9, 4.7, 7.9 Hz), 7.40 (d, 1H, J=3.2 Hz), 7.76 (dt, 1H, J=2, 7.9 Hz), 7.88 (dd, 1H, J=0.6, 1.8 Hz), 8.26 (s, 1H), 8.45 (dd, 1H, J=1.5, 5 Hz), 8.56 (d, 1H, J=1.8 Hz), 8.69 (s, 1H), 8.98 (t, 1H, J=6.2 Hz); MS (ESI) m/z=421 (MH⁺).

### Example 65

### 3-Chloro-6-furan-2-yl-8-trifluoromethyl-imidazo[1,2-a]pyridine-2-carboxylic Acid (pyridin-4-ylmethyl)-amide (Compound 165)

Prepared using similar procedure as for compound 158.

¹H NMR (d₆-DMSO, 300 MHz) δ 4.52 (d, 2H, J=6.2 Hz), 6.70 (dd, 1H, J=1.8, 3.5 Hz), 7.32 (dd, 2H, J=1.8, 4.7 Hz), 7.41 (d, 1H, J=3.5 Hz), 7.88 (dd, 1H, J=0.6, 1.8 Hz), 8.27 (s, 1H), 8.50 (dd, 2H, J=1.8, 4.7 Hz), 8.71 (s, 1H), 9.01 (t, 1H, J=6.2 Hz); MS (ESI) m/z=421 (MH⁺).

### Example 66

### [(3-Chloro-6-furan-2-yl-8-trifluoromethyl-imidazo[1,2-a]pyridine-2-carbonyl)-amino]-thiophen-2-yl-acetic Acid Methyl Ester (Compound 166)

Prepared using similar procedure as for compound 158.

¹H NMR (d₆-DMSO, 300 MHz) δ 3.72 (s, 3H), 5.93 (d, 1H, J=7.3 Hz), 6.70 (dd, 1H, J=1.8, 3.5 Hz), 7.01 (dd, 1H, J=3.5, 5 Hz), 7.18 (ddd, 1H, J=0.9, 1.2, 3.5 Hz), 7.41 (d, 1H, J=3.2 Hz), 7.51 (dd, 1H, J=1.2, 5 Hz), 7.88 (d, 1H, J=1.2 Hz), 8.28 (s, 1H), 8.70 (s, 1H), 8.80 (d, 1H, J=7.3 Hz); MS (ESI) m/z=484 (MH⁺).

### Example 67

### 3-Chloro-6-furan-2-yl-8-trifluoromethyl-imidazo[1,2-a]pyridine-2-carboxylic Acid N′-phenyl-hydrazide (Compound 167)

Prepared using similar procedure as for compound 158.

¹H NMR (d₆-DMSO, 300 MHz) δ 6.68-6.78 (m, 3H), 6.70 (dd, 1H, J=1.8, 3.5 Hz), 7.12-7.18 (m, 2H), 7.41 (d, 1H, J=3.5 Hz), 7.88 (dd, 1H, J=0.6, 1.8 Hz), 7.93 (d, 1H, J=2.6 Hz), 8.27 (s, 1H), 8.71 (s, 1H), 10.18 (d, 1H, J=2.6 Hz); MS (ESI) m/z=421 (MH⁺).

### Example 68

### [(3-Chloro-6-furan-2-yl-8-trifluoromethyl-imidazo[1,2-a]pyridine-2-carbonyl)-amino]-thiophen-2-yl-acetic Acid (Compound 168

To a stirred solution of [(3-chloro-6-furan-2-yl-8-trifluoromethyl-imidazo[1,2-a]pyridine-2-carbonyl)-amino]-thiophen-2-yl-acetic acid methyl ester (146.8 mg, 0.303 mmol) in THF (6 mL) and MeOH (2 mL) was added a solution of LiOH.H₂O (19.1 mg, 0.455 mmol) in water (1 mL) at room temperature. After 15 min, 2N HCl (0.2 mL) was added followed by removal of organic solvent under reduced pressure. The residue was diluted with 1N HCl (10 mL) and extracted with EtOAc (2×75 mL). The organic phase was dried (Na₂SO₄), filtered and concentrated to give [(3-chloro-6-furan-2-yl-8-trifluoromethyl-imidazo[1,2-a]pyridine-2-carbonyl)-amino]-thiophen-2-yl-acetic acid (146.7 mg) as a light yellow solid. ¹H NMR (d₆-DMSO, 300 MHz) δ 5.78 (d, 1H, J=7.2 Hz), 6.70 (dd, 1H, J=1.8, 3.5 Hz), 7.01 (dd, 1H, J=3.5, 5.2 Hz), 7.15 (dt, 1H, J=0.9, 3.5 Hz), 7.41 (d, 1H, J=3.2 Hz), 7.48 (dd, 1H, J=1.5, 5 Hz), 7.88 (dd, 1H, J=0.6, 1.8 Hz), 8.29 (s, 1H), 8.55 (d, 1H, J=7.2 Hz), 8.70 (s, 1H); MS (ESI) m/z=470 (MH⁺).

### Example 69

### 3-Chloro-6-furan-2-yl-8-trifluoromethyl-imidazo[1,2-a]pyridine-2-carboxylic Acid cyclopropylmethyl-amide (Compound 169)

Prepared using similar procedure as for compound 158.

¹H NMR (d₆-DMSO, 300 MHz) δ 0.25-0.47 (m, 4H), 1.09 (m, 1H), 3.17 (t, 2H, J=6.4 Hz), 6.70 (dd, 1H, J=1.8, 3.5 Hz), 7.39 (d, 1H, J=3.2 Hz), 7.88 (d, 2H, J=1.8 Hz), 8.25 (s, 1H), 8.32 (t, 1H, J=5.9 Hz), 8.70 (s, 1H); MS (ESI) m/z=384 (MH⁺).

### Example 70

### 3-Chloro-6-furan-2-yl-8-trifluoromethyl-imidazo[1,2-a]pyridine-2-carboxylic Acid cyclohexylmethyl-amide (Compound 170)

Prepared using similar procedure as for compound 158.

¹H NMR (d₆-DMSO, 300 MHz) δ 0.85-1.70 (m, 11H), 3.15 (t, 2H, J=6.5 Hz), 6.69 (dd, 1H, J=1.8, 3.5 Hz), 7.39 (d, 1H, J=3.5 Hz), 7.87 (d, 1H, J=1.8 Hz), 8.22 (t, 1H, J=6.5 Hz), 8.25 (s, 1H), 8.69 (s, 1H); MS (ESI) m/z=426.1 (MH⁺).

### Example 71

### 3-Chloro-6-furan-2-yl-8-trifluoromethyl-imidazo[1,2-a]pyridine-2-carboxylic Acid[(3-morpholin-4-yl-propylcarbamoyl)-thiophen-2-yl-methyl]-amide (Compound 171)

[(3-Chloro-6-furan-2-yl-8-trifluoromethyl-imidazo[1,2-a]pyridine-2-carbonyl)-amino]-thiophen-2-yl-acetic acid was coupled to 3-morpholin-4-yl-propylamine under standard amide bond coupling conditions to give 3-chloro-6-furan-2-yl-8-trifluoromethyl-imidazo[1,2-a]pyridine-2-carboxylic acid [(3-morpholin-4-yl-propylcarbamoyl)-thiophen-2-yl-methyl]-amide. ¹H NMR (d₆-DMSO, 300 MHz) δ 1.85 (m, 2H), 2.90-3.32 (m, 8H), 3.38-3.97 (m, 6H), 5.87 (d, 1H, J=7.6 Hz), 6.70 (dd, 1H, J=1.8, 3.5 Hz), 7.01 (dd, 1H, J=3.5, 5.2 Hz), 7.15 (brd, 1H, J=3.2 Hz), 7.41 (d, 1H, J=3.5 Hz), 7.47 (dd, 1H, J=1.2, 5 Hz), 7.89 (d, 1H, J=1.5 Hz), 8.30 (s, 1H), 8.42 (d, 1H, J=7.6 Hz), 8.70 (s, 1H), 8.76 (t, 1H, J=6.2 Hz), 9.92 (s, 1H); MS (ESI) m/z=596.1 (MH⁺).

### Example 72

### 3-Chloro-6-furan-2-yl-8-trifluoromethyl-imidazo[1,2-a]pyridine-2-carboxylic Acid [(2-dimethylamino-ethylcarbamoyl)-thiophen-2-yl-methyl]-amide (Compound 172)

[(3-Chloro-6-furan-2-yl-8-trifluoromethyl-imidazo[1,2-a]pyridine-2-carbonyl)-amino]-thiophen-2-yl-acetic acid was coupled to N,N-dimethylethylenediamine under standard amide bond coupling conditions to give 3-chloro-6-furan-2-yl-8-trifluoromethyl-imidazo[1,2-a]pyridine-2-carboxylic acid [(2-dimethylamino-ethylcarbamoyl)-thiophen-2-yl-methyl]-amide. ¹H NMR (d₆-DMSO, 300 MHz) δ 2.79 (t, 6H, J=4.4 Hz), 3.10-3.90 (m, 4H), 5.89 (d, 1H, J=7.6 Hz), 6.70 (dd, 1H, J=1.8, 3.5 Hz), 7.01 (dd, 1H, J=3.5, 5 Hz), 7.16 (dt, 1H, J=1.2, 2.9 Hz), 7.41 (d, 1H, J=3.2 Hz), 7.47 (dd, 1H, J=1.5, 5 Hz), 7.88 (d, 1H, J=1.2 Hz), 8.29 (s, 1H), 8.48 (d, 1H, J=7.6 Hz), 8.70 (s, 1H), 8.84 (t, 1H, J=6.2 Hz), 9.65 (s, 1H); MS (ESI) m/z=540.1 (MH⁺).

### Example 73

### 3-Chloro-6-furan-2-yl-8-trifluoromethyl-imidazo[1,2-a]pyridine-2-carboxylic Acid(thiophen-3-ylmethyl)-amide (Compound 173)

Prepared using similar procedure as for compound 158.

¹H NMR (d₆-DMSO, 300 MHz) δ 4.47 (d, 2H, J=6.2 Hz), 6.69 (dd, 1H, J=1.8, 3.5 Hz), 7.10 (dd, 1H, J=1.2, 5 Hz), 7.31 (dd, 1H, J=1.2, 3 Hz), 7.39 (d, 1H, J=3.2 Hz), 7.46 (dd, 1H, J=3, 5 Hz), 7.88 (dd, 1H, J=0.6, 1.8 Hz), 8.25 (s, 1H), 8.69 (s, 1H), 8.77 (t, 1H, J=6.2 Hz); MS (ESI) m/z=426 (MH⁺).

### Example 74

### 3-Chloro-6-furan-2-yl-8-trifluoromethyl-imidazo[1,2-a]pyridine-2-carboxylic Acid Benzylamide (Compound 174)

Prepared using similar procedure as for compound 158.

¹H NMR (d₆-DMSO, 300 MHz) δ 4.49 (d, 2H, J=6.2 Hz), 6.69 (dd, 1H, J=1.8, 3.2 Hz), 7.20-7.34 (m, 5H), 7.39 (d, 1H, J=3.2 Hz), 7.88 (dd, 1H, J=0.6, 1.8 Hz), 8.25 (brs, 1H), 8.70 (s, 1H), 8.86 (t, 1H, J=6.2 Hz); MS (ESI) m/z=420 (MH⁺).

### Example 75

### 3-Chloro-6-thiophen-2-yl-8-trifluoromethyl-imidazo[1,2-a]pyridine-2-carboxylic Acid(thiophen-2-ylmethyl)-amide (Compound 175)

Prepared using similar procedure as for compound 157.

¹H NMR (d₆-DMSO, 300 MHz) δ 4.63 (d, 2H, J=6.0 Hz), 6.96 (m, 1H), 7.02 (d, 1H, J=2.4 Hz), 7.21 (m, 1H), 7.37 (dd, 1H, J=1.2, 4.8 Hz), 7.70 (d, 1H, J=4.8 Hz), 7.83 (d, 1H, J=3.6 Hz), 8.15 (s, 1H), 8.69 (s, 1H), 8.89 (t, 1H, J=5.7 Hz); MS (ESI) m/z=442 (MH⁺).

### Example 76

### 3-Chloro-6-(5-chloro-thiophen-2-yl)-8-trifluoromethyl-imidazo[1,2-a]pyridine-2-carboxylic Acid (thiophen-2-ylmethyl)-amide (Compound 176)

Prepared using similar procedure as for compound 157.

¹H NMR (d₆-DMSO, 300 MHz) δ 4.63 (d, 2H, J=6.3 Hz), 6.94 (m, 1H), 7.02 (d, 1H, J=3.0 Hz), 7.26 (d, 1H, J=4.2 Hz), 7.37 (dd, 1H, J=0.9, 4.8 Hz), 7.70 (d, 1H, J=3.9 Hz), 8.12 (s, 1H), 8.69 (s, 1H), 8.90 (t, 1H, J=6.0 Hz); MS (ESI) m/z=477 (MH⁺).

### Example 77

### 3-Chloro-6-phenyl-8-trifluoromethyl-imidazo[1,2-a]pyridine-2-carboxylic Acid (thiophen-2-ylmethyl)-amide (Compound 177)

Prepared using similar procedure as for compound 157.

¹H NMR (d₆-DMSO, 300 MHz) δ 4.63 (d, 2H, J=6.0 Hz), 6.94 (m, 1H), 7.02 (d, 1H, J=2.4 Hz), 7.36 (m, 1H), 7.55-7.46 (m, 3H), 7.86 (d, 1H, J=6.9 Hz), 8.19 (s, 1H), 8.78 (s, 1H), 8.91 (t, 1H, J=6.0 Hz); MS (ESI) m/z=436 (MH⁺).

### Example 78

### 3-Chloro-6-(4-fluoro-phenyl)-8-trifluoromethyl-imidazo[1,2-a]pyridine-2-carboxylic Acid(thiophen-2-ylmethyl)-amide (Compound 178)

Prepared using similar procedure as for compound 157.

¹H NMR (d₆-DMSO, 300 MHz) δ 4.63 (d, 2H, J=6.0 Hz), 6.94 (m, 1H), 7.02 (d, 1H, J=3.6 Hz), 7.39-7.33 (m, 3H), 7.95-7.89 (m, 2H), 8.18 (s, 1H), 8.79 (s, 1H), 8.89 (t, 1H, J=6.2 Hz); MS (ESI) m/z=454 (MH⁺).

### Example 79

### 3-Chloro-6-furan-2-yl-8-trifluoromethyl-imidazo[1,2-a]pyridine-2-carboxylic Acid 2-trifluoromethyl-benzylamide (Compound 179)

Prepared using similar procedure as for compound 158.

¹H NMR (d₆-DMSO, 300 MHz) δ 4.70 (d, 2H, J=6.2 Hz), 6.70 (dd, 1H, J=1.8, 3.5 Hz), 7.41 (d, 1H, J=3.2 Hz), 7.42-7.50 (m, 2H), 7.65 (t, 1H, J=7.6 Hz), 7.73 (d, 1H, J=7.9 Hz), 7.88 (dd, 1H, J=0.6, 1.8 Hz), 8.28 (s, 1H), 8.71 (s, 1H), 8.97 (t, 1H, J=6.2 Hz); MS (ESI) m/z=488 (MH⁺).

### Example 80

### 3-Chloro-6-furan-2-yl-8-trifluoromethyl-imidazo[1,2-a]pyridine-2-carboxylic Acid 3-trifluoromethyl-benzylamide (Compound 180)

Prepared using similar procedure as for compound 158.

¹H NMR (d₆-DMSO, 300 MHz) δ 4.57 (d, 2H, J=6.2 Hz), 6.70 (dd, 1H, J=1.8, 3.5 Hz), 7.40 (d, 1H, J=3.2 Hz), 7.53-7.70 (m, 4H), 7.88 (d, 1H, J=2 Hz), 8.26 (s, 1H), 8.69 (s, 1H), 9.02 (t, 1H, J=6.2 Hz); MS (ESI) m/z=488 (MH⁺).

### Example 81

### 3-Chloro-6-furan-2-yl-8-trifluoromethyl-imidazo[1,2-a]pyridine-2-carboxylic Acid 4-trifluoromethyl-benzylamide (Compound 181)

Prepared using similar procedure as for compound 158.

¹H NMR (d₆-DMSO, 300 MHz) δ 4.57 (d, 2H, J=6.2 Hz), 6.70 (dd, 1H, J=1.8, 3.2 Hz), 7.40 (d, 1H, J=3.2 Hz), 7.54 (d, 2H, J=8 Hz), 7.69 (d, 2H, J=8 Hz), 7.88 (dd, 1H, J=0.6, 1.8 Hz), 8.26 (m, 1H), 8.70 (s, 1H), 9.01 (t, 1H, J=6.2 Hz); MS (ESI) m/z=488 (MH⁺).

### Example 82

### 3-Chloro-6-furan-2-yl-8-trifluoromethyl-imidazo[1,2-a]pyridine-2-carboxylic Acid (thiazol-2-ylmethyl)-amide (Compound 182)

Prepared using similar procedure as for compound 158.

¹H NMR (d₆-DMSO, 300 MHz) δ 4.78 (d, 2H, J=6.4 Hz), 6.70 (dd, 1H, J=1.8, 3.5 Hz), 7.40 (d, 1H, J=3.2 Hz), 7.61 (d, 1H, J=3.2 Hz), 7.72 (d, 1H, J=3.5 Hz), 7.88 (dd, 1H, J=0.6, 1.8 Hz), 8.27 (s, 1H), 8.71 (s, 1H), 9.17 (t, 1H, J=6.2 Hz); MS (ESI) m/z=427 (MH⁺).

### Example 83

### 3-Chloro-6-furan-2-yl-8-trifluoromethyl-imidazo[1,2-a]pyridine-2-carboxylic Acid (1-methyl-1H-pyrrol-2-ylmethyl)-amide (Compound 183)

Prepared using similar procedure as for compound 158.

¹H NMR (d₆-DMSO, 300 MHz) δ 3.60 (s, 3H), 4.47 (d, 2H, J=6.2 Hz), 5.88 (dd, 1H, J=1.6, 3.5 Hz), 5.99 (dd, 1H, J=1.8, 3.5 Hz), 6.64 (dd, 1H, J=2, 2.7 Hz), 6.69 (dd, 1H, J=1.8, 3.5 Hz), 7.39 (d, 1H, J=3.2 Hz), 7.87 (dd, 1H, J=0.6, 1.8 Hz), 8.25 (s, 1H), 8.41 (t, 1H, J=5.9 Hz), 8.64 (s, 1H); MS (ESI) m/z=423.1 (MH⁺).

### Example 84

### 3-Chloro-6-furan-2-yl-8-trifluoromethyl-imidazo[1,2-a]pyridine-2-carboxylic Acid(tetrahydro-furan-2-ylmethyl)-amide (Compound 184)

Prepared using similar procedure as for compound 158.

¹H NMR (d₆-DMSO, 300 MHz) δ 1.60 (m, 1H), 1.75-1.97 (m, 3H), 3.26-3.45 (m, 2H), 3.60-3.81 (m, 2H), 4.02 (m, 1H), 6.69 (dd, 1H, J=1.8, 3.5 Hz), 7.39 (d, 1H, J=3.5 Hz), 7.87 (d, 1H, J=1.5 Hz), 8.11 (t, 1H, J=6 Hz), 8.26 (s, 1H), 8.69 (s, 1H); MS (ESI) m/z=414.1 (MH⁺).

### Example 85

### 3-Chloro-6-furan-2-yl-8-trifluoromethyl-imidazo[1,2-a]pyridine-2-carboxylic Acid (2-thiophen-2-yl-ethyl)-amide (Compound 185)

Prepared using similar procedure as for compound 158.

¹H NMR (d₆-DMSO, 300 MHz) δ 3.09 (t, 2H, J=7 Hz), 3.56 (q, 2H, J=7 Hz), 6.70 (dd, 1H, J=1.8, 3.5 Hz), 6.92 (dd, 1H, J=1.2, 3.5 Hz), 6.95 (dd, 1H, J=3.5, 5 Hz), 7.34 (dd, 1H, J=1.2, 5 Hz), 7.39 (d, 1H, J=3.5 Hz), 7.88 (d, 1H, J=1.5 Hz), 8.25 (s, 1H), 8.41 (t, 1H, J=6.2 Hz), 8.69 (s, 1H); MS (ESI) m/z=440 (MH⁺).

### Example 86

### (3-Chloro-6-furan-2-yl-8-trifluoromethyl-imidazo[1,2-a]pyridin-2-yl)-(3-phenyl-pyrrolidin-1-yl)-methanone (Compound 186)

Prepared using similar procedure as for compound 158.

¹H NMR (d₆-DMSO, 300 MHz) δ 1.99-2.38 (m, 2H), 3.40-4.10 (m, 4.5H), 4.26 (dd, 0.5H, J=8, 11 Hz), 6.69 (dd, 0.5H, J=1.8, 3.2 Hz), 6.70 (dd, 0.5H, J=1.8, 3.5 Hz), 7.20-7.40 (m, 6H), 7.87 (d, 0.5H, 1.2 Hz), 7.88 (d, 0.5H, J=1.8 Hz), 8.21 (s, 0.5H), 8.24 (s, 0.5H), 8.69 (s, 0.5H), 8.71 (s, 0.5H); MS (ESI) m/z=460.1 (MH⁺).

### Example 87

### 3-Chloro-6-furan-2-yl-8-trifluoromethyl-imidazo[1,2-a]pyridine-2-carboxylic Acid Indan-1-ylamide (Compound 187)

Prepared using similar procedure as for compound 158.

¹H NMR (d₆-DMSO, 300 MHz) δ 2.13 (ddd, 1H, J=8.8, 12.6, 17 Hz), 2.44 (m, 1H), 2.86 (dt, 1H, J=8.2, 15.5 Hz), 3.02 (ddd, 1H, J=3, 9, 15.5 Hz), 5.58 (q, 1H, J=8.5 Hz), 6.70 (dd, 1H, J=1.8, 3.5 Hz), 7.15-7.29 (m, 4H), 7.40 (d, 1H, J=3.2 Hz), 7.88 (dd, 1H, J=0.6, 1.8 Hz), 8.25 (s, 1H), 8.49 (d, 1H, J=9 Hz), 8.71 (s, 1H); MS (ESI) m/z=446.1 (MH⁺).

### Example 88

### 3-Chloro-6-furan-2-yl-8-trifluoromethyl-imidazo[1,2-a]pyridine-2-carboxylic Acid (2-phenyl-cyclopropyl)-amide (Compound 188)

Prepared using similar procedure as for compound 158.

¹H NMR (d₆-DMSO, 300 MHz) δ 1.25 (dt, 1H, J=5.9, 8 Hz), 1.51 (dt, 1H, J=5, 9 Hz), 2.21 (ddd, 1H, J=3.5, 6.5, 9.7 Hz), 3.04 (m, 1H), 6.69 (dd, 1H, J=1.8, 3.2 Hz), 7.14-7.30 (m, 5H), 7.39 (d, 1H, J=3.2 Hz), 7.87 (d, 1H, J=1.8 Hz), 8.25 (s, 1H), 8.54 (d, 1H, J=4.7 Hz), 8.69 (s, 1H); MS (ESI) m/z=446.1 (MH⁺).

### Example 89

### (3-Chloro-6-furan-2-yl-8-trifluoromethyl-imidazo[1,2-a]pyridin-2-yl)-(2-thiophen-2-yl-pyrrolidin-1-yl)-methanone (Compound 189)

Prepared using similar procedure as for compound 158.

¹H NMR (d₆-DMSO, 300 MHz) δ 1.92-2.5 (m, 5H), 3.63-4.11 (m, 2H), 5.56 (dd, 0.55H, J=1.9, 8.2 Hz), 6.17 (dd, 0.45H, J=3.5, 7 Hz), 6.56 (brd, 0.55H, J=3.2 Hz), 6.67 (m, 1.6H), 6.96 (dd, 0.55H, J=3.5, 5 Hz), 7.01 (dt, 0.55H, J=0.9, 3.5 Hz), 7.18 (dd, 0.45H, J=1.2, Hz), 7.34-7.37 (m, 1H), 7.39 (d, 0.55H, J=3.2 Hz), 7.85 (d, 0.45H, J=1.5 Hz), 7.87 (d, 0.55H, J=1.5 Hz), 8.20 (s, 0.45H), 8.23 (s, 0.55H), 8.58 (s, 0.45H), 8.70 (s, 0.55H); MS (ESI) m/z=466 (MH⁺).

### Example 90

### 3-Chloro-6-furan-2-yl-8-trifluoromethyl-imidazo[1,2-a]pyridine-2-carboxylic Acid 2-methoxy-benzylamide (Compound 190)

Prepared using similar procedure as for compound 158.

¹H NMR (d₆-DMSO, 300 MHz) δ 3.85 (s, 3H), 4.48 (d, 2H, J=6.2 Hz), 6.70 (dd, 1H, J=2, 3.2 Hz), 6.89 (dt, 1H, J=0.9, 7.3 Hz), 7.00 (dd, 1H, J=0.9, 8.2 Hz), 7.15-7.27 (m, 2H), 7.40 (d, 1H, J=3.2 Hz), 7.88 (dd, 1H, J=0.6, 1.8 Hz), 8.26 (s, 1H), 8.59 (t, 1H, J=6 Hz), 8.70 (s, 1H); MS (ESI) m/z=450.1 (MH⁺).

### Example 91

### 3-Chloro-6-furan-2-yl-8-trifluoromethyl-imidazo[1,2-a]pyridine-2-carboxylic Acid 3-methoxy-benzylamide (Compound 191)

Prepared using similar procedure as for compound 158.

¹H NMR (d₆-DMSO, 300 MHz) δ 3.73 (s, 3H), 4.46 (d, 2H, J=6.5 Hz), 6.69 (dd, 1H, J=2, 3.5 Hz), 6.80 (ddd, 1H, J=0.9, 2.6, 8.2 Hz), 6.89-6.94 (m, 2H), 7.23 (t, 1H, J=8.2 Hz), 7.39 (d, 1H, J=3.2 Hz), 7.87 (dd, 1H, J=0.6, 1.8 Hz), 8.25 (s, 1H), 8.69 (s, 1H), 8.82 (t, 1H, J=6.5 Hz); MS (ESI) m/z=450 (MH⁺).

### Example 92

### 3-Chloro-6-furan-2-yl-8-trifluoromethyl-imidazo[1,2-a]pyridine-2-carboxylic Acid 4-methoxy-benzylamide (Compound 192)

Prepared using similar procedure as for compound 158.

¹H NMR (d₆-DMSO, 300 MHz) δ 3.72 (s, 3H), 4.41 (d, 2H, J=6.2 Hz), 6.69 (dd, 1H, J=1.8, 3.5 Hz), 6.87 (brd, 2h, J=8.8 Hz), 7.27 (brd, 2H, J=8.8 Hz), 7.39 (d, 1H, J=3.5 Hz), 7.87 (d, 1H, J=1.5 Hz), 8.25 (s, 1H), 8.69 (s, 1H), 8.75 (t, 1H, J=6.2 Hz); MS (ESI) m/z=450.1 (MH⁺).

### Example 93

### 6-Phenyl-3,8-bis-trifluoromethyl-imidazo[1,2-a]pyridine-2-carboxylic Acid (thiophen-2-ylmethyl)-amide (Compound 193)

### Step 1: 6-Phenyl-3,8-bis-trifluoromethyl-imidazo[1,2-a]pyridine-2-carboxylic Acid Ethyl Ester

A mixture of 3-bromo-6-phenyl-8-trifluoromethyl-imidazo[1,2-a]pyridine-2-carboxylic acid ethyl ester (206.6 mg, 0.5 mmol), methyl 2-chloro-2,2-difluoroacetate (123 μL, 1.15 mmol), copper(I) iodide (114.3 mg, 0.6 mmol), and potassium fluoride (35 mg, 0.6 mmol) was heated in DMF (1.25 mL) at 120° C. for 15 hours in a sealed tube. The mixture was diluted with EtOAc (20 mL) and washed with saturated aqueous NH₄Cl (10 mL), then brine (10 mL). The organic phase was dried (Na₂SO₄), filtered and concentrated. Column chromatography of the crude material gave 6-phenyl-3,8-bis-trifluoromethyl-imidazo[1,2-a]pyridine-2-carboxylic acid ethyl ester (43.2 mg, 21%). ¹H NMR (d₆-DMSO, 300 MHz) δ 1.35 (t, 3H, J=7 Hz), 4.42 (q, 2H, J=7 Hz), 7.47-7.58 (m, 3H), 7.82-7.85 (m, 2H), 8.36 (s, 1H), 8.83 (s, 1H); MS (ESI) m/z=403.1 (MH⁺).

### Step 2: 6-Phenyl-3,8-bis-trifluoromethyl-imidazo[1,2-a]pyridine-2-carboxylic Acid(thiophen-2-ylmethyl)-amide (Compound 193)

6-Phenyl-3,8-bis-trifluoromethyl-imidazo[1,2-a]pyridine-2-carboxylic acid ethyl ester (41.5 mg, 0.1 mmol) was hydrolyzed in ACN (10 mL) and 6N HCl (10 mL) at 100° C. for 24 hours. The solvents were removed to give a precipitate which was triturated with water to give the acid which was used for the next step without further purification. The acid was coupled with 2-thiophenemethylamine under standard amide coupling conditions to give 6-phenyl-3,8-bis-trifluoromethyl-imidazo[1,2-a]pyridine-2-carboxylic acid (thiophen-2-ylmethyl)-amide. ¹H NMR (d₆-DMSO, 300 MHz) δ 4.66 (d, 2H, J=6.2 Hz), 6.97 (dd, 1H, J=3.5, 4.8 Hz), 7.04 (dd, 1H, J=0.9, 3.5 Hz), 7.41 (dd, 1H, J=1.3, 4.8 Hz), 7.45-7.58 (m, 3H), 7.81-7.85 (m, 2H), 8.33 (s, 1H), 8.81 (s, 1H), 9.21 (t, 1H, J=6.2 Hz); MS (ESI) m/z=470 (MH⁺).

### Example 94

### 3-Ethyl-6-furan-2-yl-8-trifluoromethyl-imidazo[1,2-a]pyridine-2-carboxylic Acid (thiophen-2-ylmethyl)-amide (Compound 194)

Using similar procedure as for the preparation of compound 156

¹H NMR (d₆-DMSO, 300 MHz) [symbol] 8.82 (s, 1H), 8.67 (t, 1H, J=6.3 Hz), 8.09 (s, 1H), 7.84 (s, 1H), 7.35 (d, 1H, J=1.5 Hz), 7.34 (d, 1H, J=0.9 Hz), 7.29 (d, 1H, J=3.6 Hz), 7.01 (m, 1H), 6.93 (m, 1H), 6.67 (dd, 1H, J=2.1, 3.6 Hz), 4.63 (d, 2H, J=6.3 Hz), 3.42 (q, 2H, J=7.6 Hz), 1.20 (t, 3H, J=7.5 Hz); MS (ESI) m/z=420.1 (MH⁺).

### Example 95

### (3-Chloro-6-furan-2-yl-8-trifluoromethyl-imidazo[1,2-a]pyridin-2-yl)-carbamic Acid Tert-butyl Ester (Compound 195)

3-Chloro-6-furan-2-yl-8-trifluoromethyl-imidazo[1,2-a]pyridine-2-carboxylic acid (2.01 g, 6.1 mmol) was dissolved in tert-butanol (20 mL), triethylamine (2.6 mL, 18.3 mmol) and diphenylphosphoryl azide (DPPA, 3.35 g, 12.2 mmol) were added and the mixture refluxed for 14 hours. The solvent was removed under reduced pressure and the mixture partitioned between ethyl acetate and 5% aqueous NaHCO₃. The organic layer was washed (water, brine) and dried and the crude product was purified by silica gel chromatography to afford (3-chloro-6-furan-2-yl-8-trifluoromethyl-imidazo[1,2-a]pyridin-2-yl)-carbamic acid tert-butyl ester as a light brown solid (1.2 gm, 50%). ¹H NMR (d₆-DMSO, 300 MHz) [symbol] 9.56 (s, 1H), 8.64 (s, 1H), 8.11 (s, 1H), 7.83 (d, 1H, J=1.8 Hz), 7.31 (d, 1H, J=3.3 Hz), 6.66 (dd, 1H, J=1.5, 3.3 Hz), 1.45 (s, 9H); MS (ESI) m/z=402.1 (MH⁺).

### Example 96

### 3-Chloro-6-(3-fluoro-phenyl)-8-trifluoromethyl-imidazo[1,2-a]pyridine-2-carboxylic Acid(thiophen-2-ylmethyl)-amide (Compound 196)

Prepared using similar procedure as for compound 157.

¹H NMR (d₆-DMSO, 300 MHz) δ 4.63 (d, 2H, J=6.3 Hz), 6.96-6.93 (m, 1H), 7.25 (dd, 1H, J=0.6, 3.3 Hz), 7.29 (dt, 1H, J=2.4, 8.7 Hz), 7.36 (dd, 1H, J=0.6, 4.8 Hz), 7.59-7.52 (m, 1H), 7.72 (d, 1H, J=8.1 Hz), 7.80 (m, 1H), 8.22 (bs, 1H), 8.86 (s, 1H), 8.89 (t, 1H, J=6.3 Hz); MS (ESI) m/z=454 (MH⁺).

### Example 97

### 3-Chloro-6-(2-fluoro-phenyl)-8-trifluoromethyl-imidazo[1,2-a]pyridine-2-carboxylic Acid(thiophen-2-ylmethyl)-amide (Compound 197)

Prepared using similar procedure as for compound 157.

¹H NMR (d₆-DMSO, 300 MHz) δ 4.63 (d, 2H, J=6.3 Hz), 6.96-6.93 (m, 1H), 7.02 (m, 1H), 7.43-7.34 (m, 3H), 7.54-7.49 (m, 1H), 7.76 (dt, 1H, J=1.8, 7.5 Hz), 8.09 (s, 1H), 8.77 (s, 1H), 8.91 (t, 1H, J=6.3 Hz); MS (ESI) m/z=454 (MH⁺).

### Example 98

### 3-Chloro-6-(3,4-difluoro-phenyl)-8-trifluoromethyl-imidazo[1,2-a]pyridine-2-carboxylic Acid(thiophen-2-ylmethyl)-amide (Compound 198)

Prepared using similar procedure as for compound 157.

¹H NMR (d₆-DMSO, 300 MHz) δ 4.63 (d, 2H, J=6.0 Hz), 6.96-6.93 (m, 1H), 7.02 (dd, 1H, J=0.9, 3.0 Hz), 8.08 (ddd, 1H, J=2.4, 8.1, 12.0 Hz), 7.36-7.54 (m, 1H), 7.79-7.74 (m, 1H), 7.54-7.49 (m, 1H), 7.76 (dt, 1H, J=1.8, 7.5 Hz), 8.22 (s, 1H), 8.87 (s, 1H), 8.90 (t, 1H, J=6.3 Hz); MS (ESI) m/z=472 (MH⁺).

### Example 99

### 3-Chloro-8-trifluoromethyl-6-(4-trifluoromethyl-phenyl)-imidazo[1,2-a]pyridine-2-carboxylic Acid (thiophen-2-ylmethyl)-amide (Compound 199)

Prepared using similar procedure as for compound 157.

¹H NMR (d₆-DMSO, 300 MHz) δ 4.63 (d, 2H, J=6.0 Hz), 6.96-6.93 (m, 1H), 7.02 (d, 1H, J=3.0 Hz), 7.36 (dd, 1H, J=1.2, 4.8 Hz), 7.87 (d, 1H, J=8.1 Hz), 8.11 (d, 1H, J=8.4 Hz), 8.22 (s, 1H), 8.26 (s, 1H), 8.90 (s, 1H), 8.92 (t, 1H, J=6.3 Hz); MS (ESI) m/z=504 (MH⁺).

### Example 100

### 3,6-Di-thiophen-3-yl-8-trifluoromethyl-imidazo[1,2-a]pyridine-2-carboxylic Acid (thiophen-2-ylmethyl)-amide (Compound 200)

Prepared using similar procedure as for compound 157.

¹H NMR (d₆-DMSO, 300 MHz) δ 4.61 (d, 2H, J=6.0 Hz), 6.93 (dd, 1H, J=3.3, 5.1 Hz), 6.99 (d, 1H, J=2.4 Hz), 7.34 (dd, 1H, J=1.2, 5.1 Hz), 7.45 (dd, 1H, J=1.2, 4.8 Hz), 7.57 (dd, 1H, J=1.2, 4.8 Hz), 7.68 (dd, 1H, J=3.0, 5.1 Hz), 7.72 (dd, 1H, J=3.0, 5.1 Hz), 8.09 (dd, 1H, J=1.2, 3.0 Hz), 8.12 (dd, 1H, J=1.5, 3.0 Hz), 8.19 (s, 1H), 8.57 (s, 1H), 8.77 (t, 1H, J=6.3 Hz); MS (ESI) m/z=490 (MH⁺).

### Example 101

### 3-Chloro-6-furan-2-yl-8-trifluoromethyl-imidazo[1,2-a]pyridine-2-carboxylic Acid 2-fluoro-benzylamide (Compound 201)

Prepared using similar procedure as for compound 158.

¹H NMR (d₆-DMSO, 300 MHz) δ 4.55 (d, 2H, J=6.2 Hz), 6.70 (dd, 1H, J=2, 3.5 Hz), 7.13-7.21 (m, 2H), 7.26-7.40 (m, 3H), 7.88 (d, 1H, J=1.5 Hz), 8.26 (s, 1H), 8.70 (s, 1H), 8.83 (t, 1H, J=6.5 Hz); MS (ESI) m/z=438 (MH⁺).

### Example 102

### 3-Chloro-6-furan-2-yl-8-trifluoromethyl-imidazo[1,2-a]pyridine-2-carboxylic Acid 2-trifluoromethoxy-benzylamide (Compound 202)

Prepared using similar procedure as for compound 158.

¹H NMR (d₆-DMSO, 300 MHz) δ 4.58 (d, 2H, J=6.2 Hz), 6.70 (dd, 1H, J=1.8, 3.5 Hz), 7.32-7.44 (m, 5H), 7.88 (dd, 1H, J=0.6, 1.8 Hz), 8.27 (s, 1H), 8.70 (s, 1H), 8.87 (t, 1H, J=6.2 Hz); MS (ESI) m/z=504 (MH⁺).

### Example 103

### 3-Chloro-6-furan-2-yl-8-trifluoromethyl-imidazo[1,2-a]pyridine-2-carboxylic Acid 3-trifluoromethoxy-benzylamide (Compound 203)

Prepared using similar procedure as for compound 158.

¹H NMR (d₆-DMSO, 300 MHz) δ 4.53 (d, 2H, J=6.2 Hz), 6.70 (dd, 1H, J=1.8, 3.5 Hz), 7.22-7.40 (m, 3H), 7.46 (t, 1H, J=8 Hz), 7.88 (dd, 1H, J=0.6, 1.8 Hz), 8.26 (s, 1H), 8.70 (s, 1H), 8.98 (t, 1H, J=6.2 Hz); MS (ESI) m/z=504 (MH⁺).

### Example 104

### 3-Chloro-6-furan-2-yl-8-trifluoromethyl-imidazo[1,2-a]pyridine-2-carboxylic Acid 4-trifluoromethoxy-benzylamide (Compound 204)

Prepared using similar procedure as for compound 158.

¹H NMR (d₆-DMSO, 300 MHz) δ 4.51 (d, 2H, J=6.2 Hz), 6.70 (dd, 1H, J=1.8, 3.5 Hz), 7.28-7.34 (m, 2H), 7.40 (d, 1H, J=3.2 Hz), 7.45 (brd, 2H, J=8.8 Hz), 7.88 (dd, 1H, J=0.6, 1.8 Hz), 8.26 (s, 1H), 8.70 (s, 1H), 8.95 (t, 1H, J=6.2 Hz); MS (ESI) m/z=504

### Example 105

### N-(3-chloro-6-furan-2-yl-8-trifluoromethyl-imidazo[1,2-a]pyridin-2-yl)-2-phenyl-acetamide (Compound 205)

(3-Chloro-6-furan-2-yl-8-trifluoromethyl-imidazo[1,2-a]pyridin-2-yl)-carbamic acid tert-butyl ester (0.1 gm, 0.26 mmol) in THF (1 mL) was added to a suspension of sodium hydride (60%, 0.073 g, 1.83 mmol) in THF (5 mL). The mixture was stirred for 15 min and phenyl acetyl chloride was added and the mixture refluxed for 14 hours. The mixture was partitioned between ethyl acetate and water and the organic layer was washed (water, brine) and dried to afford the crude product. This was redissloved in dichloromethane (3 mL), trifluoroacetic acid (3 mL) was added and the mixture stirred for 4 h. The crude mixture was purified by silica gel chromatography followed by washing with 1N HCl and acetonitrile to afford N-(3-chloro-6-furan-2-yl-8-trifluoromethyl-imidazo[1,2-a]pyridine-2-yl)-2-phenyl-acetamide (0.016 g, 11%). ¹H NMR (d₆-DMSO, 300 MHz) [symbol] 10.73 (s, 1H), 8.65 (s, 1H), 8.13 (s, 1H), 7.84 (s, 1H), 7.30, m, 6H), 6.66 (dd, 1H, J=2.1, 3.6 Hz), 3.69 (s, 2H); MS (ESI) m/z=420.0 (MH⁺).

### Example 106

### 5-(Chloro-difluoro-methyl)-7-furan-2-yl-imidazo[1,2-a]pyridine-2-carboxylic Acid(thiophen-2-ylmethyl)-amide (Compound 206)

### Step 1: 6-(Chloro-difluoro-methyl)-4-furan-2-yl-pyridin-2-ylamine

To a suspension of 6-(chloro-difluoro-methyl)-4-furan-2-yl-pyridine-2-carboxylic acid (300 mg, 1.096 mmol) in tert-butanol (7.5 mL) was added triethylamine (229 μL, 1.645 mmol) followed by diphenylphosphoryl azide (354 μL, 1.645 mmol). The mixture was then heated at 85° C. for 17 hours. Upon cooling, the solvent was removed under reduced pressure. The crude material was diluted with EtOAc (25 mL) and washed with saturated aqueous NaHCO₃ (10 mL), then brine (10 mL). The organic phase was dried (Na₂SO₄), filtered and concentratedto give a brown oil. The crude material was heated in 3N HCl (10 mL) under reflux for 6 hours. Upon cooling, the upper yellow solution was removed, and the aqueous phase was concentrated under reduced pressure. To the residue was added Et₂O (30 mL) and 1N NaOH (5 mL). The aqueous phase was separated and extracted again with Et₂O (30 mL). The combined organic extracts were dried (Na₂SO₄), filtered and concentrated to give 6-(chloro-difluoro-methyl)-4-furan-2-yl-pyridin-2-ylamine (59 mg) as a beige solid. ¹H NMR (d₆-DMSO, 300 MHz) δ 6.65 (dd, 1H, J=1.8, 3.5 Hz), 6.66 (brs, 2H), 6.87 (d, 1H, J=1.2 Hz), 7.13 (d, 1H, J=1.2 Hz), 7.25 (dd, 1H, J=0.9, 3.5 Hz), 7.85 (dd, 1H, J=0.9, 1.8 Hz); MS (ESI) m/z=245 (MH⁺).

### Step 2:-5-(chloro-difluoro-methyl)-7-furan-2-yl-imidazo[1,2-a]pyridine-2-carboxylic Acid Ethyl Ester

6-(Chloro-difluoro-methyl)-4-furan-2-yl-pyridin-2-ylamine (49.6 mg) was treated with ethyl bromopyruvate in DMF under similar conditions as for the preparation of compound 151 to give 5-(chloro-difluoro-methyl)-7-furan-2-yl-imidazo[1,2-a]pyridine-2-carboxylic acid ethyl ester (36.7 mg, 53%) as a yellow solid. ¹H NMR (d₆-DMSO, 300 MHz) δ 1.35 (t, 3H, J=7 Hz), 4.36 (q, 2H, J=7 Hz), 6.72 (dd, 1H, J=1.8, 3.5 Hz), 7.46 (d, 1H, J=3.5 Hz), 7.93 (dd, 1H, J=1.8, 3.5 Hz), 8.12 (s, 1H), 8.35 (s, 1H); MS (ESI) m/z=341 (MH⁺).

### Step 3: 5-(Chloro-difluoro-methyl)-7-furan-2-yl-imidazo[1,2-a]pyridine-2-carboxylic Acid(thiophen-2-ylmethyl)-amide (Compound 206)

5-(Chloro-difluoro-methyl)-7-furan-2-yl-imidazo[1,2-a]pyridine-2-carboxylic acid ethyl ester was hydrolyzed in 1,4-dioxane (1 mL) and 6N HCl (2 mL) at 125° C. for 30 min under microwave conditions. The solvents were removed under reduced pressure to give the acid which was used for the next step without further purification. The acid was coupled to 2-thiophenemethylamine under standard coupling conditions to give 5-(chloro-difluoro-methyl)-7-furan-2-yl-imidazo[1,2-a]pyridine-2-carboxylic acid (thiophen-2-ylmethyl)-amide (13.1 mg) as a yellow solid. ¹H NMR (d₆-DMSO, 300 MHz) δ 4.64 (d, 2H, J=6.2 Hz), 6.72 (dd, 1H, J=1.8, 3.2 Hz), 6.94 (dd, 1H, J=3.2, 5 Hz), 7.02 (dd, 1H, J=1.2, 3.2 Hz), 7.37 (dd, 1H, J=1.2, 5 Hz), 7.46 (d, 1H, J=1.2, 5 Hz), 7.46 (d, 1H, J=3.2 Hz), 7.91 (d, 2H, J=1.2 Hz), 8.01 (s, 1H), 8.27 (s, 1H), 9.21 (t, 1H, J=6.2 Hz); MS (ESI) m/z=408 (MH⁺).

### Example 107

### 3-Chloro-6-pyridin-4-yl-8-trifluoromethyl-imidazo[1,2-a]pyridine-2-carboxylic Acid(thiophen-2-lmethyl)-amide (Compound 207)

Prepared using similar procedure as for compound 157.

¹H NMR (d₆-DMSO, 300 MHz) δ 4.70 (d, 2H, J=6.3 Hz), 7.02 (dd, 1H, J=3.6, 5.1 Hz), 7.10 (d, 1H, J=3.3 Hz), 7.43 (dd, 1H, J=0.6, 4.5 Hz), 8.46 (t, 1H, J=6.3 Hz), 8.96 (d, 1H, J=6.6 Hz), 9.0 (t, 1H, J=6.0 Hz), 9.25 (s, 1H); MS (ESI) m/z=437 (MH⁺).

### Example 108

### 3-Chloro-6-pyridin-3-yl-8-trifluoromethyl-imidazo[1,2-a]pyridine-2-carboxylic Acid(thiophen-2-lmethyl)-amide (Compound 208)

Prepared using similar procedure as for compound 157.

¹H NMR (d₆-DMSO, 300 MHz) δ 4.64 (d, 2H, J=6.0 Hz), 6.96-6.93 (m, 1H), 7.06 (d, 1H, J=3.6 Hz), 7.37 (dd, 1H, J=1.5, 5.4 Hz), 7.83 (dd, 1H, J=5.4, 8.1 Hz), 8.33 (s, 1H), 8.63 (d, 1H, J=7.8 Hz), 8.79 (dd, 1H, J=1.5, 5.4 Hz), 8.94 (t, 1H, J=6.3 Hz), 9.07 (s, 1H), 9.23 (d, 1H, J=2.4 Hz); MS (ESI) m/z=437 (MH⁺).

### Example 109

### 3-Chloro-6-(4-methyl-thiophen-3-yl)-8-trifluoromethyl-imidazo[1,2-a]pyridine-2-carboxylic Acid (thiophen-2-ylmethyl)-amide (Compound 209)

Prepared using similar procedure as for compound 157.

¹H NMR (d₆-DMSO, 300 MHz) δ 2.29 (s, 3H), 4.63 (d, 2H, J=6.3 Hz), 6.95 (dd, 1H, J=3.6, 4.8 Hz), 7.01 (d, 1H, J=2.4 Hz), 7.36 (m, 1H), 7.83 (d, 1H, J=3.3 Hz), 7.98 (s, 1H), 8.57 (s, 1H), 8.88 (t, 1H, J=6.3 Hz); MS (ESI) m/z=456 (MH⁺).

### Example 110

### 3-Chloro-6-(3,5-dimethyl-isoxazol-4-yl)-8-trifluoromethyl-imidazo[1,2-a]pyridine-2-carboxylic Acid (thiophen-2-ylmethyl)-amide (Compound 210)

Prepared using similar procedure as for compound 157.

¹H NMR (d₆-DMSO, 300 MHz) [symbol] 2.25 (s, 3H), 2.44 (s, 3H), 4.63 (d, 2H, J=6.0 Hz), 6.95 (m, 1H), 7.0 (s, 1H), 7.37 (d, 1H, J=4.2 Hz), 7.95 (s, 1H), 7.98 (s, 1H), 8.68 (s, 1H), 8.90 (t, 1H, J=6.3 Hz); MS (ESI) m/z=455 (MH⁺).

### Example 111

### 1-(3-Chloro-6-furan-2-yl-8-trifluoromethyl-imidazo[1,2-a]pyridin-2-yl)-3-phenyl-urea (Compound 211)

(3-Chloro-6-furan-2-yl-8-trifluoromethyl-imidazo[1,2-a]pyridin-2-yl)-carbamic acid tert-butyl ester (0.117 gm, 0.29 mmol) in THF (1 mL) was added to a suspension of sodium hydride (60%, 0.08 g, 2.04 mmol) in THF (5 mL). The mixture was stirred for 15 min and phenyl isocyanate was added and the mixture refluxed for 14 hours. The mixture was partitioned between ethyl acetate and water and the organic layer was washed (water, brine) and dried to afford the crude product. (MS analysis of the crude product indicated that the BOC protecting group had got removed under the reaction conditions.) The product was purified by suspending the crude mixture in acetonitrile and aqueous 1N HCl and washing the solids further with aqueous acid to afford 1-(3-cloro-6-furan-2-yl-8-trifluoromethyl-imidazo[1,2-a]pyridin-2-yl)-3-phenyl-urea (0.01 g, 8%). ¹H NMR (d₆-DMSO, 300 MHZ) [symbol] 9.81 (s, 1H), 9.46 (s, 1H), 8.65 (s, 1H), 8.14 (s, 1H), 7.84 (d, 1H, J=1.5 Hz), 7.45 (d, 2H, J=8.7 Hz), 7.32 (m, 3H), 6.99 (t, 1H, J=7.5 Hz), 6.67 (dd, 1H, J=1.8, 3.6 Hz); MS (ESI) m/z=421.0 (MH⁺).

### Example 112

### 3-Chloro-6-furan-2-yl-8-trifluoromethyl-imidazo[1,2-a]pyridine-2-carboxylic Acid 4-morpholin-4-yl-benzylamide (Compound 212)

### Standard HATU Coupling Conditions:

A mixture of 3-chloro-6-furan-2-yl-8-trifluoromethyl-imidazo[1,2-a]pyridine-2-carboxylic acid (40 mg, 0.1210 mmol), 4-morpholinobenzylamine (27.9 mg, 0.1452 mmol), HATU (55.2 mg, 0.1452 mmol), and di-isopropylethylamine (84.3 μL, 0.4839 mmol) was stirred in DMF (0.8 mL) at room temperature. After 1.5 hours, the mixture was diluted with EtOAc (20 mL) and washed with saturated aqueous NaHCO₃ (10 mL), then brine (10 mL). The filtrate was dried (Na₂SO₄), filtered and concentrated. Column chromatography [n-hex/EtOAc (5:4 v/v)] of the crude material gave 3-chloro-6-furan-2-yl-8-trifluoromethyl-imidazo[1,2-a]pyridine-2-carboxylic acid 4-morpholin-4-yl-benzylamide (compound 212) (51.1 mg, 84%) as a white powder. ¹H NMR (d₆-DMSO, 300 MHz) δ 3.03-3.08 (m, 4H), 3.69-3.74 (m, 4H), 4.39 (d, 2H, J=6.2 Hz), 6.69 (dd, 1H, J=1.6, 3.2 Hz), 6.89 (d, 2H, J=8.8 Hz), 7.21 (d, 2H, J=8.5 Hz), 7.39 (d, 1H, J=3.2 Hz), 7.87 (d, 1H, J=1.2 Hz), 8.24 (brs, 1H), 8.68 (t, 1H, J=6.2 Hz), 8.69 (brs, 1H); MS (ESI) m/z=505.1 (MH⁺).

### Example 113

### 3-Chloro-6-furan-2-yl-8-trifluoromethyl-imidazo[1,2-a]pyridine-2-carboxylic Acid 3-morpholin-4-yl-benzylamide (Compound 213)

Using standard HATU coupling conditions, 3-chloro-6-furan-2-yl-8-trifluoromethyl-imidazo[1,2-a]pyridine-2-carboxylic acid, and (3-morpholinophenyl)methylamine gave 3-chloro-6-furan-2-yl-8-trifluoromethyl-imidazo[1,2-a]pyridine-2-carboxylic acid 3-morpholin-4-yl-benzylamide (compound 213). ¹H NMR (d₆-DMSO, 300 MHz) δ 3.06-3.10 (m, 4H), 3.70-3.75 (m, 4H), 4.44 (d, 2H, J=6.1 Hz), 6.70 (dd, 1H, J=1.8, 3.5 Hz), 6.77-6.83 (m, 2H), 6.94 (brs, 1H), 7.17 (t, 1H, J=8 Hz), 7.39 (d, 1H, J=3.5 Hz), 7.88 (d, 1H, J=1.2 Hz), 8.25 (brs, 1H), 8.70 (brs, 1H), 8.74 (t, 1H, J=6.1 Hz); MS (ESI) m/z=505.1 (MH⁺).

### Example 114

### 3-Chloro-6-furan-2-yl-8-trifluoromethyl-imidazo[1,2-a]pyridine-2-carboxylic Acid 4-(2-dimethylamino-ethoxy)-benzylamide (Compound 214)

Using standard HATU coupling conditions, 3-chloro-6-furan-2-yl-8-trifluoromethyl-imidazo[1,2-a]pyridine-2-carboxylic acid, and 4-(2-(dimethylamino)ethoxy)benzylamine gave 3-chloro-6-furan-2-yl-8-trifluoromethyl-imidazo[1,2-a]pyridine-2-carboxylic acid 4-(2-dimethylamino-ethoxy)-benzylamide (compound 214). ¹H NMR (d₆-DMSO, 300 MHz) δ 2.82 (s, 6H), 3.46 (t, 2H, J=5 Hz), 4.29 (t, 2H, J=5 Hz), 4.43 (d, 2H, J=6.4 Hz), 6.70 (dd, 1H, J=1.8, 3.5 Hz), 6.95 (d, 2H, J=8.5 Hz), 7.31 (d, 2H, J=8.5 Hz), 7.39 (d, 1H, J=3.5 Hz), 7.88 (d, 1H, J=1.8 Hz), 8.26 (s, 1H), 8.70 (s, 1H), 8.73 (t, 1H, J=6.1 Hz), 9.84 (s, 1H); MS (ESI) m/z=507.1 (MH⁺).

### Example 115

### 3-Chloro-6-furan-2-yl-8-trifluoromethyl-imidazo[1,2-a]pyridine-2-carboxylic Acid 2-(2-dimethylamino-ethoxy)-benzylamide (Compound 215)

Using standard HATU coupling conditions, 3-chloro-6-furan-2-yl-8-trifluoromethyl-imidazo[1,2-a]pyridine-2-carboxylic acid, and 2-(2-(dimethylamino)ethoxy)benzylamine gave 3-chloro-6-furan-2-yl-8-trifluoromethyl-imidazo[1,2-a]pyridine-2-carboxylic acid 2-(2-dimethylamino-ethoxy)-benzylamide (compound 215). ¹H NMR (d₆-DMSO, 300 MHz) δ 2.93 (s, 6H), 3.56 (brs, 2H), 4.36 (t, 2H, J=5 Hz), 4.56 (d, 2H, J=6.2 Hz), 6.70 (dd, 1H, J=1.8, 3.5 Hz), 6.97 (dt, 1H, J=0.6, 7.5 Hz), 7.02 (dd, 1H, J=0.6, 8.2 Hz), 7.24-7.32 (m, 2H), 7.40 (d, 1H, J=3.2 Hz), 7.88 (d, 1H, J=1.2 Hz), 8.27 (brs, 1H), 8.71 (s, 1H), 8.73 (t, 1H, J=6.2 Hz), 9.76 (brs, 1H); MS (ESI) m/z=507.1 (MH⁺).

### Example 116

### (3-Chloro-6-furan-2-yl-8-trifluoromethyl-imidazo[1,2-a]pyridin-2-yl)-(3-phenyl-piperidin-1-yl)-methanone (Compound 216)

Using standard HATU coupling conditions, 3-chloro-6-furan-2-yl-8-trifluoromethyl-imidazo[1,2-a]pyridine-2-carboxylic acid, and 3-phenylpiperidine gave (3-chloro-6-furan-2-yl-8-trifluoromethyl-imidazo[1,2-a]pyridin-2-yl)-(3-phenyl-piperidin-1-yl)-methanone (compound 216). ¹H NMR (d₆-DMSO, 300 MHz) δ 1.58-2.02 (m, 4H), 2.72-3.20 (m, 3H), 4.10-4.62 (m, 2H), 6.68 (dd, 0.5H, J=1.8, 3.5 Hz), 6.70 (dd, 0.5H, J=1.8, 3.5 Hz), 7.14-7.39 (m, 6H), 7.86 (d, 0.5H, J=1.1 Hz), 7.88 (d, 0.5H, J=1.5 Hz), 8.19 (s, 0.5H), 8.23 (s, 0.5H), 8.67 (s, 0.5H), 8.70 (s, 0.5H); MS (ESI) m/z=474.1 (MH⁺).

### Example 117

### (3-Chloro-6-furan-2-yl-8-trifluoromethyl-imidazo[1,2-a]pyridin-2-yl)-(5,7-dihydro-pyrrolo[3,4-b]pyridin-6-yl)-methanone (Compound 217)

Using standard HATU coupling conditions, 3-chloro-6-furan-2-yl-8-trifluoromethyl-imidazo[1,2-a]pyridine-2-carboxylic acid, and 6,7-dihydro-5H-pyrrolo[3,4-b]pyridine gave (3-chloro-6-furan-2-yl-8-trifluoromethyl-imidazo[1,2-a]pyridin-2-yl)-(5,7-dihydro-pyrrolo[3,4-b]pyridin-6-yl)-methanone (compound 217). ¹H NMR (d₆-DMSO, 300 MHz) δ 4.94 (d, 2H, J=16 Hz), 5.28 (s, 2H), 6.71 (dd, 1H, J=1.8, 3.5 Hz), 7.34-7.42 (m, 2H), 7.83-7.92 (m, 2H), 8.28 (brs, 1H), 8.50 (m, 1H), 8.74 (s, 1H); MS (ESI) m/z=433 (MH⁺).

### Example 118

### (3-Chloro-6-furan-2-yl-8-trifluoromethyl-imidazo[1,2-a]pyridin-2-yl)-(4-phenyl-piperidin-1-yl)-methanone (Compound 218)

Using standard HATU coupling conditions, 3-chloro-6-furan-2-yl-8-trifluoromethyl-imidazo[1,2-a]pyridine-2-carboxylic acid, and 4-phenylpiperidine gave (3-chloro-6-furan-2-yl-8-trifluoromethyl-imidazo[1,2-a]pyridin-2-yl)-(4-phenyl-piperidin-1-yl)-methanone (compound 218). ¹H NMR (d₆-DMSO, 300 MHz) δ 1.56-1.95 (m, 4H), 2.80-2.99 (m, 2H), 3.24 (t, 1H, J=11 Hz), 4.19 (brd, 1H, J=12.3 Hz), 4.67 (brd, 1H, J=12.6 Hz), 6.69 (dd, 1H, J=1.8, 3.5 Hz), 7.16-7.33 (m, 5H), 7.38 (d, 1H, J=3.2 Hz), 7.88 (d, 1H, J=1.2 Hz), 8.22 (s, 1H), 8.70 (s, 1H); MS (ESI) m/z=474.1 (MH⁺).

### Example 119

### 3-Chloro-6-furan-2-yl-8-trifluoromethyl-imidazo[1,2-a]pyridine-2-carboxylic Acid (5-pyridin-2-yl-thiophen-2-ylmethyl)-amide (Compound 219)

Using standard HATU coupling conditions, 3-chloro-6-furan-2-yl-8-trifluoromethyl-imidazo[1,2-a]pyridine-2-carboxylic acid, and [5-(2-pyridyl)-2-thienyl]methylamine gave 3-chloro-6-furan-2-yl-8-trifluoromethyl-imidazo[1,2-a]pyridine-2-carboxylic acid (5-pyridin-2-yl-thiophen-2-ylmethyl)-amide (compound 219). ¹H NMR (d₆-DMSO, 300 MHz) δ 4.65 (d, 2H, J=5.9 Hz), 6.69 (dd, 1H, J=1.8, 3.5 Hz), 7.04 (d, 1H, J=3.5 Hz), 7.23 (ddd, 1H, J=1.5, 5.0, 7.0 Hz), 7.39 (d, 1H, J=3.2 Hz), 7.62 (d, 1H, J=3.8 Hz), 7.79 (dt, 1H, J=1.8, 7.3 Hz), 7.85 (dt, 1H, J=1.2, 7.9 Hz), 7.88 (dd, 1H, J=0.6, 1.8 Hz), 8.26 (brs, 1H), 8.46 (ddd, 1H, J=0.8, 1.2, 4.7 Hz), 8.70 (s, 1H), 8.97 (t, 1H, J=6.3 Hz); MS (ESI) m/z=503 (MH⁺).

### Example 120

### 6-Furan-3-yl-3-[(thiophen-2-ylmethyl)-amino]-8-trifluoromethyl-imidazo[1,2-a]pyridine-2-carboxylic Acid Ethyl Ester (Compound 220)

### Step 1: 6-Bromo-3-nitro-8-trifluoromethyl-imidazo[1,2-a]pyridine-2-carboxylic Acid Ethyl Ester

A mixture of 6-bromo-8-trifluoromethyl-imidazo[1,2-a]pyridine-2-carboxylic acid ethyl ester (2 g, 5.933 mmol) was heated at 50° C. in fuming nitric acid (10 mL) and sulfuric acid (20 mL) for 5.5 hours. The mixture was cooled and poured into ice-water (400 mL) to give a precipitate which was filtered to give 6-bromo-3-nitro-8-trifluoromethyl-imidazo[1,2-a]pyridine-2-carboxylic acid ethyl ester (1.25 g, 55%) as a light yellow solid. MS (ESI) m/z=405.9 (MNa⁺).

### Step 2: 6-Furan-3-yl-3-nitro-8-trifluoromethyl-imidazo[1,2-a]pyridine-2-carboxylic Acid Ethyl Ester

A mixture of 6-bromo-3-nitro-8-trifluoromethyl-imidazo[1,2-a]pyridine-2-carboxylic acid ethyl ester (650 mg, 1.7011 mmol), furan-3-boronic acid (286 mg, 2.5517 mmol), tetrakis(triphenylphosphine)palladium(0) (98.3 mg, 0.085 mmol) in 1M K₃PO₄ (4 mL) and 1,4-dioxane (12 mL) was treated under microwave conditions at 140° C. for 5 min. The microwave reaction was repeated again and the crude reaction mixtures were combined for workup. The mixture was diluted with EtOAc (120 mL) and washed with saturated aqueous NaHCO₃ (30 mL), then brine (30 mL). The filtrate was dried (Na₂SO₄), filtered and concentrated. The crude material was absorbed on silica gel and purified by chromatography [n-hex/EtOAc (5:1 v/v) to (4:1 v/v)] to give 6-furan-3-yl-3-nitro-8-trifluoromethyl-imidazo[1,2-a]pyridine-2-carboxylic acid ethyl ester (620 mg, 49%) as a yellow solid. MS (ESI) m/z=370 (MH⁺).

### Step 3: 6-Furan-3-yl-3-[(thiophen-2-ylmethyl)-amino]-8-trifluoromethyl-imidazo[1,2-a]pyridine-2-carboxylic Acid Ethyl Ester (compound 220)

A mixture of 6-furan-3-yl-3-nitro-8-trifluoromethyl-imidazo[1,2-a]pyridine-2-carboxylic acid ethyl ester (235 mg, 0.6364 mmol) and thiophene-2-methylamine (653 μL, 0.6364 mmol) was heated at 150° C. in NMP under microwave conditions for 10 min. The crude reaction mixture was loaded on a pad of silica gel and eluted with EtOAc/n-hex. The fractions containing the product were concentrated and repurified by silica gel chromatography [n-hex/EtOAc (5:1 v/v)] to give 6-furan-3-yl-3-[(thiophen-2-ylmethyl)-amino]-8-trifluoromethyl-imidazo[1,2-a]pyridine-2-carboxylic acid ethyl ester (266 mg, 96%). MS (ESI) m/z=436.1 (MH⁺).

### Example 121

### 1-(3-Chloro-6-furan-3-yl-8-trifluoromethyl-imidazo[1,2-a]pyridine-2-carbonyl)-4-phenyl-pyrrolidine-3-carboxylic Acid Methyl Ester (Compound 221)

Using standard HATU coupling conditions, 3-chloro-6-furan-3-yl-8-trifluoromethyl-imidazo[1,2-a]pyridine-2-carboxylic acid, and 4-phenylpyrrolidine-3-methylcarboxylate gave 1-(3-chloro-6-furan-3-yl-8-trifluoromethyl-imidazo[1,2-a]pyridine-2-carbonyl)-4-phenyl-pyrrolidine-3-carboxylic acid methyl ester (compound 221). ¹H NMR (d₆-DMSO, 300 MHz) δ 3.44-3.70 (m, 2H), 3.53 (s, 1.5H), 3.57 (s, 1.5H), 3.77 (dd, 0.5H, J=9.1, 12 Hz), 3.85 (t, 0.5 H, J=10.5 Hz), 3.98-4.14 (m, 2H), 4.33 (d, 0.5H, J=7.6 Hz), 4.37 (d, 0.5H, J=7.6 Hz), 7.24-7.40 (m, 6H), 7.82 (t, 0.5H, J=1.8 Hz), 7.83 (t, 0.5H, J=1.8 Hz), 8.16 (s, 0.5H), 8.21 (s, 0.5H), 8.53 (s, 0.5H), 8.56 (s, 0.5H), 8.80 (s, 0.5H), 8.82 (s, 0.5H); MS (ESI) m/z=518.1 (MH⁺).

### Example 122

### {6-Furan-3-yl-2-[(thiophen-2-ylmethyl)-carbamoyl]-8-trifluoromethyl-imidazo[1,2-a]pyridin-3-yl}-acetic Acid Methyl Ester (Compound 222)

### Step 1: 6-Bromo-3-methoxycarbonylmethyl-8-trifluoromethyl-imidazo[1,2-a]pyridine-2-carboxylic Acid Methyl Ester

A mixture of 5-bromo-3-trifluoromethyl-pyridin-2-ylamine (2.93 g, 12.14 mmol) and 3-bromo-2-oxo-pentanedioic acid dimethyl ester (prepared from bromination of dimethyl 2-oxoglutarate) (6.15 g, 24.29 mmol) was heated in DMF at 70° C. for a week. The mixture was poured into water (700 mL) to give a precipitate which was filtered and dried to give the product (1.74 g). The filtrate was extracted with EtOAc (300 mL) which after concentration of the solvent yielded 3.71 g of crude material. The crude prduct was absorbed on silica gel followed by column chromatography [(3:1 v/v) n-hex:EtOAc)] to give 6-bromo-3-methoxycarbonylmethyl-8-trifluoromethyl-imidazo[1,2-a]pyridine-2-carboxylic acid methyl ester as a yellow solid (1.37 g). ¹H NMR (d₆-DMSO, 300 MHz) δ 3.65 (s, 3H), 3.86 (s, 3H), 4.51 (s, 2H), 8.02 (s, 1H), 8.23 (s, 1H); MS (ESI) m/z=395 (MH⁺).

### Step 2: 3-Carboxymethyl-6-furan-3-yl-8-trifluoromethyl-imidazo[1,2-a]pyridine-2-carboxylic Acid

A mixture of 6-bromo-3-methoxycarbonylmethyl-8-trifluoromethyl-imidazo[1,2-a]pyridine-2-carboxylic acid methyl ester (600 mg, 1.5185 mmol), furan-3-boronic acid (254.9 mg, 2.2778 mmol), tetrakis(triphenylphosphine)palladium(0) (87.7 mg, 0.0759 mmol) in 1M K₃PO₄ (4 mL) and 1,4-dioxane (12 mL) was treated under microwave conditions at 120° C. for 5 min. Additional K₃PO₄ (1M, 2 mL) was added to the mixture and microwaved at 120° C. for 10 min. This was repeated with additional K₃PO₄ (1M, 0.5 mL) and microwaved at 120° C. for 5 min. The solvent was removed and 10% NaOH was added (12 mL). The aqueous phase was washed with Et₂O (2×60 mL) followed by addition of 6N HCl until pH 1. The precipitate was filtered and dried under vacuum to give 3-carboxymethyl-6-furan-3-yl-8-trifluoromethyl-imidazo[1,2-a]pyridine-2-carboxylic acid (445 mg, 83%) as a beige solid. ¹H NMR (d₆-DMSO, 300 MHz) δ 4.48 (s, 2H), 7.23 (dd, 1H, J=0.8, 1.7 Hz), 7.82 (t, 1H, J=1.5 Hz), 8.12 (s, 1H), 8.47 (s, 1H), 8.98 (s, 1H); MS (ESI) m/z=355 (MH⁺).

### Step 3: 6-Furan-3-yl-3-methoxycarbonylmethyl-8-trifluoromethyl-imidazo[1,2-a]pyridine-2-carboxylic Acid

To a stirred solution of 3-carboxymethyl-6-furan-3-yl-8-trifluoromethyl-imidazo[1,2-a]pyridine-2-carboxylic acid (745 mg, 2.1031 mmol) in MeOH (150 mL) was added thionyl chloride (7.7 μL, 0.1052 mmol). Additional thionyl chloride (total of 200 μL) was added throughout the reaction. After 6 days, the solvent was concentrated to give a mixture of the mono-methyl ester and the dimethyl ester. The crude material was diluted with EtOAc (100 mL) and washed with 2N HCl, dried (Na₂SO₄), filter and concentrated to give an off-white solid (759 mg) which was used for the next step without further purification. MS (ESI) m/z=369 (MH⁺).

### Step 4: {6-Furan-3-yl-2-[(thiophen-2-ylmethyl)-carbamoyl]-8-trifluoromethyl-imidazo[1,2-a]pyridin-3-yl}-acetic Acid Methyl Ester (compound 222)

A mixture of 6-furan-3-yl-3-methoxycarbonylmethyl-8-trifluoromethyl-imidazo[1,2-a]pyridine-2-carboxylic acid (23 mg, 0.06245 mmol), thiophene-2-methylamine (7.7 μL, 0.07495 mmol), HATU (28.5 mg, 0.07495 mmol), and di-isopropylethylamine (32.6 L, 0.1847 mmol) in DMF (0.8 mL) was stirred at room temperature. After 30 min, the mixture was diluted with EtOAc (10 mL) and washed successively with 2N HCl (10 mL), saturated aqueous NaHCO₃ (10 mL), and brine (10 mL). The filtrate was dried (Na₂SO₄), filtered and concentrated. Column chromatography [n-hex/EtOAc (2:1 v/v)] of the crude product gave {6-furan-3-yl-2-[(thiophen-2-ylmethyl)-carbamoyl]-8-trifluoromethyl-imidazo[1,2-a]pyridin-3-yl}-acetic acid methyl ester (15 mg) as a white powder. ¹H NMR (d₆-DMSO, 300 MHz) δ 3.65 (s, 3H), 4.63 (s, 2H, J=7 Hz), 4.61 (s, 2H), 6.94 (dd, 1H, J=3.2, 5 Hz), 7.01 (dd, 1H, J=1.2, 3.2 Hz), 7.22 (dd, 1H, J=0.6, 1.8 Hz), 7.36 (dd, 1H, J=1.2, 5 Hz), 7.82 (t, 1H, J=1.8 Hz), 8.14 (s, 1H), 8.46 (brs, 1H), 8.77 (t, 1H, J=6.2 Hz), 8.98 (s, 1H); MS (ESI) m/z=464 (MH⁺).

### Example 123

### 1-(3-Chloro-6-furan-3-yl-8-trifluoromethyl-imidazo[1,2-a]pyridine-2-carbonyl)-4-phenyl-pyrrolidine-3-carboxylic Acid (Compound 223)

To a solution of 1-(3-chloro-6-furan-3-yl-8-trifluoromethyl-imidazo[1,2-a]pyridine-2-carbonyl)-4-phenyl-pyrrolidine-3-carboxylic acid methyl ester (201 mg, 0.3882 mmol) in THF (30 mL) and MeOH (10 mL) was added a solution of lithium hydroxide monohydrate (24.4 mg, 0.5822 mmol) in water (10 mL). After 3.5 hours, 2N HCl (2 mL) was added followed by the removal of solvent under reduced pressure. The remaining aqueous solution was extracted with EtOAc (100 mL, 20 mL). The extracts were dried (Na₂SO₄), filtered and concentrated. A portion of the crude material (50 mg) was purified by preparative HPLC (30-100% ACN gradient) to give 1-(3-chloro-6-furan-3-yl-8-trifluoromethyl-imidazo[1,2-a]pyridine-2-carbonyl)-4-phenyl-pyrrolidine-3-carboxylic acid (compound 223) (30.5 mg). The rest of the material (169 mg) was used for further reactions without purification. ¹H NMR (d₆-DMSO, 300 MHz) δ 3.00-3.80 (m, 3H), 3.96-4.13 (m, 2H), 4.30-4.37 (m, 1H), 7.20-7.41 (m, 6H), 7.82 (t, 0.5H, J=1.8 Hz), 7.83 (t, 0.5H, J=1.5 Hz), 8.16 (s, 0.5H), 8.20 (s, 0.5H), 8.53 (s, 0.5H), 8.55 (s, 0.5H), 8.79 (s, 0.5H), 8.82 (s, 0.5H), 12.53 (s, 1H); MS (ESI) m/z=504 (MH⁺).

### Example 124

### 1-(3-Chloro-6-furan-3-yl-8-trifluoromethyl-imidazo[1,2-a]pyridine-2-carbonyl)-4-phenyl-pyrrolidine-3-carboxylic Acid (2-dimethylamino-ethyl)-amide (Compound 224)

Using standard HATU coupling conditions, 1-(3-chloro-6-furan-3-yl-8-trifluoromethyl-imidazo[1,2-a]pyridine-2-carbonyl)-4-phenyl-pyrrolidine-3-carboxylic acid (compound 223), and N,N-dimethylethylenediamine gave 1-(3-chloro-6-furan-3-yl-8-trifluoromethyl-imidazo[1,2-a]pyridine-2-carbonyl)-4-phenyl-pyrrolidine-3-carboxylic acid (2-dimethylamino-ethyl)-amide (compound 224). ¹H NMR (d₆-DMSO, 300 MHz) δ 2.64 (s, 3H), 2.70 (s, 3H), 2.90-3.48 (m, 5H), 3.58-4.40 (m, 5H), 7.20-7.37 (m, 6H), 7.82 (t, 0.5H, J=1.8 Hz), 7.84 (t, 0.5H, J=1.8 Hz), 8.17 (s, 0.5H), 8.20 (s, 0.5H), 8.31-8.42 (m, 1H), 8.53 (s, 0.5H), 8.55 (s, 0.5H), 8.80 (s, 0.5H), 8.82 (s, 0.5H); MS (ESI) m/z=574.2 (MH⁺).

### Example 125

### 1-(3-Chloro-6-furan-3-yl-8-trifluoromethyl-imidazo[1,2-a]pyridine-2-carbonyl)-4-phenyl-pyrrolidine-3-carboxylic Acid (Compound 225)

Using standard HATU coupling conditions, 1-(3-chloro-6-furan-3-yl-8-trifluoromethyl-imidazo[1,2-a]pyridine-2-carbonyl)-4-phenyl-pyrrolidine-3-carboxylic acid (compound 223), and 4-(2-aminoethyl)morpholine gave 1-(3-chloro-6-furan-3-yl-8-trifluoromethyl-imidazo[1,2-a]pyridine-2-carbonyl)-4-phenyl-pyrrolidine-3-carboxylic acid (compound 225). ¹H NMR (d₆-DMSO, 300 MHz) δ 2.80-4.40 (m, 18H), 7.20-7.38 (m, 6H), 7.82 (t, 0.5H, J=1.8 Hz), 7.84 (t, 0.5H, J=1.8 Hz), 8.17 (s, 0.5H), 8.20 (s, 0.5H), 8.32-8.45 (m, 1H), 8.53 (s, 0.5H), 8.55 (s, 0.5H), 8.80 (s, 0.5H), 8.82 (s, 0.5H); MS (ESI) m/z=616.2 (MH⁺).

### Example 126

### {6-Furan-3-yl-2-[(thiophen-2-ylmethyl)-carbamoyl]-8-trifluoromethyl-imidazo[1,2-a]pyridin-3-yl}-acetic Acid (Compound 226)

To a solution of {6-furan-3-yl-2-[(thiophen-2-ylmethyl)-carbamoyl]-8-trifluoromethyl-imidazo[1,2-a]pyridin-3-yl}-acetic acid methyl ester (compound 222) (48.5 mg, 0.1047 mmol) in THF (6 mL) and water (2 mL) was added lithium hydroxide monohydrate (6.6 mg, 0.1570 mmol) in water (0.1 mL). After 35 min, 2N HCl was added to acidify the solution followed by concentration of solvent. The remaining aqueous solution was extracted with EtOAc (20 mL). The organic phase was separated, dried (Na₂SO₄), filtered and concentrated. Column chromatography [n-hex/EtOAc (2:1 v/v) followed by n-hex/EtOAc (1:2 v/v), then MeOH/EtOAc (5:95 v/v)] of the crude material gave {6-furan-3-yl-2-[(thiophen-2-ylmethyl)-carbamoyl]-8-trifluoromethyl-imidazo[1,2-a]pyridin-3-yl}-acetic acid (16 mg, 34%) as a white solid.

¹H NMR (d₆-DMSO, 300 MHz) δ 4.57 (s, 2H), 4.63 (d, 2H, J=6.2 Hz), 6.94 (dd, 1H, J=3.7, 5.1 Hz), 7.02 (dd, 1H, J=1.1, 3.3 Hz), 7.24 (dd, 1H, J=0.7, 1.8 Hz), 7.36 (dd, 1H, J=1.7, 3.2 Hz), 7.82 (t, 1H, J=1.8 hz), 8.13 (s, 1H), 8.47 (s, 1H), 8.75 (t, 1H, J=6.2 Hz), 8.97 (s, 1H); MS (ESI) m/z=450 (MH⁺).

### Example 127

### 1-(3-Chloro-6-furan-3-yl-8-trifluoromethyl-imidazO [1,2-a]pyridine-2-carbonyl)-3-phenyl-pyrrolidine-2-carboxylic Acid Methyl Ester (Compound 227)

Using standard HATU coupling conditions, 3-chloro-6-furan-3-yl-8-trifluoromethyl-imidazo[1,2-a]pyridine-2-carboxylic acid, and methyl-3-phenylpyrrolidine-2-carboxylate gave 1-(3-chloro-6-furan-3-yl-8-trifluoromethyl-imidazo[1,2-a]pyridine-2-carbonyl)-3-phenyl-pyrrolidine-2-carboxylic acid methyl ester (compound 227). ¹H NMR (d₆-DMSO, 300 MHz) δ 2.00-2.40 (m, 2H), 3.30-4.40 (m, 3H), 3.55 (s, 1.5H), 3.61 (s, 1.5H), 4.49 (d, 0.5H, J=8.5 Hz), 5.36 (d, 0.5H, J=4.4 Hz), 7.20-7.38 (m, 6H), 7.82 (t, 0.5H, J=1.8 Hz), 7.83 (t, 0.5H, J=1.8 Hz), 8.16 (s, 0.5H), 8.22 (s, 0.5H), 8.53 (s, 0.5H), 8.56 (s, 0.5H), 8.79 (s, 0.5H), 8.82 (s, 0.5H); MS (ESI) m/z=518.1 (MH⁺).

### Example 128

### 1-(3-Chloro-6-furan-3-yl-8-trifluoromethyl-imidazo[1,2-a]pyridine-2-carbonyl)-3-phenyl-pyrrolidine-2-carboxylic Acid (Compound 228)

1-(3-Chloro-6-furan-3-yl-8-trifluoromethyl-imidazo[1,2-a]pyridine-2-carbonyl)-3-phenyl-pyrrolidine-2-carboxylic acid methyl ester was saponified using lithium hydroxide to give 1-(3-chloro-6-furan-3-yl-8-trifluoromethyl-imidazo[1,2-a]pyridine-2-carbonyl)-3-phenyl-pyrrolidine-2-carboxylic acid (compound 228). MS (ESI) m/z=504.1 (MH⁺).

### Example 129

### 1-(3-Chloro-6-furan-3-yl-8-trifluoromethyl-imidazo[1,2-a]pyridine-2-carbonyl)-2-phenyl-pyrrolidine-2-carboxylic Acid (Compound 229)

Using standard HATU coupling conditions, 3-chloro-6-furan-3-yl-8-trifluoromethyl-imidazo[1,2-a]pyridine-2-carboxylic acid, and 2-phenyl-pyrrolidine-2-carboxylic acid gave 1-(3-chloro-6-furan-3-yl-8-trifluoromethyl-imidazo[1,2-a]pyridine-2-carbonyl)-2-phenyl-pyrrolidine-2-carboxylic acid (compound 229). MS (ESI) m/z=504.1 (MH⁺).

### Example 130

### 1-(3-Chloro-6-furan-3-yl-8-trifluoromethyl-imidazo[1,2-a]pyridine-2-carbonyl)-3-phenyl-pyrrolidine-2-carboxylic Acid (2-dimethylamino-ethyl)-amide (Compound 230)

Using standard HATU coupling conditions, 1-(3-chloro-6-furan-3-yl-8-trifluoromethyl-imidazo[1,2-a]pyridine-2-carbonyl)-3-phenyl-pyrrolidine-2-carboxylic acid (compound 228), and N,N-dimethylethylenediamine gave 1-(3-chloro-6-furan-3-yl-8-trifluoromethyl-imidazo[1,2-a]pyridine-2-carbonyl)-3-phenyl-pyrrolidine-2-carboxylic acid (2-dimethylamino-ethyl)-amide (compound 230). ¹H NMR (d₆-DMSO, 300 MHz) δ 2.16-2.40 (m, 2H), 2.79 (s, 3H), 2.80 (s, 3H), 3.00-4.30 (m, 7H), 4.44 (d, 1H, J=7.6 Hz), 7.20-7.36 (m, 5H), 7.84 (t, 1H, J=2 Hz), 8.23 (s, 1H), 8.36 (t, 1H, J=5.8 Hz), 8.56 (s, 1H), 9.22 (s, 1H), 9.29 (s, 1H); MS (ESI) m/z=574.2 (MH⁺).

### Example 131

### 1-(3-Chloro-6-furan-3-yl-8-trifluoromethyl-imidazo[1,2-a]pyridine-2-carbonyl)-3-phenyl-pyrrolidine-2-carboxylic acid (2-morpholin-4-yl-ethyl)-amide (Compound 231)

Using standard HATU coupling conditions, 1-(3-chloro-6-furan-3-yl-8-trifluoromethyl-imidazo[1,2-a]pyridine-2-carbonyl)-3-phenyl-pyrrolidine-2-carboxylic acid (compound 228), and 4-(2-aminoethyl)morpholine gave 1-(3-chloro-6-furan-3-yl-8-trifluoromethyl-imidazo[1,2-a]pyridine-2-carbonyl)-3-phenyl-pyrrolidine-2-carboxylic acid (2-morpholin-4-yl-ethyl)-amide (compound 231). MS (ESI) m/z=616.2 (MH⁺).

### Example 132

### 6-Furan-3-yl-3-nitro-8-trifluoromethyl-imidazo[1,2-a]pyridine-2-carboxylic Acid (thiophen-2-ylmethyl)-amide (Compound 232)

### Step 1: 6-Bromo-3-nitro-8-trifluoromethyl-imidazo[1,2-a]pyridine-2-carboxylic Acid

To a solution of 6-bromo-8-trifluoromethyl-imidazo[1,2-a]pyridine-2-carboxylic acid (2.1 g, 6.7952 mmol) in cone H₂SO₄ (20 mL) at 0° C. was added fuming HNO₃ (5 mL) dropwise. The solution was then heated to 50° C. After 10 hours, the mixture was cooled to room temperature and stirred overnight. The mixture was carefully poured into ice-water (200 mL) to give a precipitate which was filtered and dried under high vacuum to give 6-bromo-3-nitro-8-trifluoromethyl-imidazo[1,2-a]pyridine-2-carboxylic acid (1.8844 g, 78%) as a light yellow solid. ¹H NMR (d₆-DMSO, 300 MHz) δ 8.52 (s, 1H), 9.49 (d, 1H, J=1.8 Hz); MS (ESI) m/z=355.9 (MH⁺).

### Step 2: 6-Bromo-3-nitro-8-trifluoromethyl-imidazo[1,2-a]pyridine-2-carboxylic Acid(thiophen-2-ylmethyl)-amide

A mixture of 6-bromo-3-nitro-8-trifluoromethyl-imidazo[1,2-a]pyridine-2-carboxylic acid (1 g, 2.8508 mmol), thiophene-2-methylamine (322 μL, 3.1359 mmol), HATU (1.192 g, 3.1359 mmol), and di-isopropylethylamine (1.49 mL, 8.5524 mmol in DMF (12 mL) was stirred at room temperature. After 45 min, 0.3 eq of HATU and 0.3 eq of thiophene-2-methylamine were added. After 20 min, the mixture was diluted with EtOAc (150 mL) and washed successively with 2N HCl (2×50 mL), saturated aqueous NaHCO₃ (50 mL), and brine (50 mL). The filtrate was dried (Na₂SO₄), filtered and concentrated to give a brown solid which was absorbed on silica gel. Column chromatography [n-hex/EtOAc (3:1 v/v)] of the crude material 6-bromo-3-nitro-8-trifluoromethyl-imidazo[1,2-a]pyridine-2-carboxylic acid (thiophen-2-ylmethyl)-amide (0.85 g, 66%) as a yellow solid.

### Step 3: 6-Furan-3-yl-3-nitro-8-trifluoromethyl-imidazo[1,2-a]pyridine-2-carboxylic Acid(thiophen-2-ylmethyl)-amide (compound 232)

A mixture of 6-bromo-3-nitro-8-trifluoromethyl-imidazo[1,2-a]pyridine-2-carboxylic acid (thiophen-2-ylmethyl)-amide (600 mg, 1.3357 mmol), furan-3-boronic acid (224 mg, 2.0035 mmol), tetrakis(triphenylphosphine)palladium(0) (77.2 mg, 0.06678 mmol) in 1M K₃PO₄ (3 mL) and 1,4-dioxane (9 mL) was treated under microwave conditions at 120° C. for 5 min. The mixture was diluted with EtOAc (100 mL) and washed with saturated aqueous NaHCO₃ (30 mL), then brine (30 mL). The filtrate was dried (Na₂SO₄), filtered and concentrated to give the crude material which was column chromatographed [n-hex/EtOAc (3:1 v/v) to n-hex/EtOAc (2:1 v/v)] to give 6-furan-3-yl-3-nitro-8-trifluoromethyl-imidazo[1,2-a]pyridine-2-carboxylic acid (thiophen-2-ylmethyl)-amide (418.6 mg, 72%) as a yellow powder. ¹H NMR (d₆-DMSO, 300 MHz) δ 4.69 (d, 2H, J=5.9 Hz), 6.99 (dd, 1H, J=3.5, 5 Hz), 7.09 (dd, 1H, J=0.6, 3.2 Hz), 7.22 (dd, 1H, J=0.6, 1.8 Hz), 7.45 (dd, 1H, J=1.2, 5 Hz), 7.84 (t, 1H, J=1.8 Hz), 8.53 (s, 1H), 8.61 (s, 1H), 9.32 (t, 1H, J=5.9 Hz), 9.50 (brs, 1H); MS (ESI) m/z=437 (MH⁺).

### Example 133

### (3-Chloro-6-furan-3-yl-8-trifluoromethyl-imidazo[1,2-a]pyridin-2-yl)-[3-(4-fluoro-phenyl)-pyrrolidin-1-yl]-methanone (Compound 233)

### Step 1: 6-Bromo-3-chloro-8-trifluoromethyl-imidazo[1,2-a]pyridine-2-carboxylic Acid

A suspension of 6-bromo-3-chloro-8-trifluoromethyl-imidazo[1,2-a]pyridine-2-carboxylic acid ethyl ester (5.05 g, 13.59 mmol) was heated under reflux in 3N HCl (100 mL) and acetonitrile (100 mL) for 3 days. Upon cooling, the solvent was removed followed by addition of 10% NaOH until pH˜10. The mixture was washed with Et₂O (2×80 mL) and acidified with 6N HCl to precipitate a white solid which was filtered and dried under high vacuum to give 6-bromo-3-chloro-8-trifluoromethyl-imidazo[1,2-a]pyridine-2-carboxylic acid (4.3 g, 92%). ¹H NMR (d₆-DMSO, 300 MHz) δ 8.07 (m, 1H), 8.97 (m, 1H), 13.45 (brs, 1H); MS (ESI) m/z=344.9 (MH⁺).

### Step 2: (6-Bromo-3-chloro-8-trifluoromethyl-imidazo[1,2-a]pyridin-2-yl)-[3-(4-fluoro-phenyl)-pyrrolidin-1-yl]-methanone

A mixture of 6-bromo-3-chloro-8-trifluoromethyl-imidazo[1,2-a]pyridine-2-carboxylic acid (937.3 mg, 2.7289 mmol), 3-(4-fluorophenyl)pyrrolidine (541 mg, 3.2746 mmol), HATU (1.25 g, 3.2746 mmol), and di-isopropylethylamine (1.9 mL, 10.9154 mmol) in DMF (14 mL) was stirred at room temperature. After 2 hours, the mixture was diluted with EtOAc (125 mL) and washed successively with 2N HCl (50 mL), saturated aqueous NaHCO₃ (50 mL), and brine (50 mL). The filtrate was dried (Na₂SO₄), filtered and concentrated. Column chromatography [n-hex/EtOAc (5:4 v/v)] of the crude material gave (6-bromo-3-chloro-8-trifluoromethyl-imidazo[1,2-a]pyridin-2-yl)-[3-(4-fluoro-phenyl)-pyrrolidin-1-yl]-methanone (1.17 g, 87%) as a foam. ¹H NMR (d₆-DMSO, 300 MHz) [symbol] 1.96-2.34 (m, 2H), 3.38-4.08 (m, 4.5H), 4.19 (dd, 0.5H, J=7.3-11.4 Hz), 7.15 (q, 2H, J=8.8 Hz), 7.31-7.42 (m, 2H), 8.05 (m, 0.5H), 8.07 (m, 0.5H), 8.97 (m, 0.5H), 8.99 (m, 0.5H); MS (ESI) m/z=490, 492 (MH⁺).

### Step 3: (3-Chloro-6-furan-3-yl-8-trifluoromethyl-imidazo[1,2-a]pyridin-2-yl)-[3-(4-fluoro-phenyl)-pyrrolidin-1-yl]-methanone (compound 233)

A mixture of (6-bromo-3-chloro-8-trifluoromethyl-imidazo[1,2-a]pyridin-2-yl)-[3-(4-fluoro-phenyl)-pyrrolidin-1-yl]-methanone (55 mg, 0.1122 mmol), furan-3-boronic acid (18.8 mg, 0.1681 mmol), tetrakis(triphenylphosphine)palladium(0) (6.5 mg, 0.0056 mmol) in 1M K₃PO₄ (0.4 mL) and 1,4-dioxane (1.2 mL) was treated under microwave conditions at 100° C. for min. The mixture was diluted with EtOAc (20 mL) and washed with saturated aqueous NaHCO₃ (10 mL), and brine (10 mL). Column chromatography [n-hex/EtOAc (5:4 v/v)] of the crude material gave (3-chloro-6-furan-3-yl-8-trifluoromethyl-imidazo[1,2-a]pyridin-2-yl)-[3-(4-fluoro-phenyl)-pyrrolidin-1-yl]-methanone (49 mg, 91%) as an off-white powder. ¹H NMR (d₆-DMSO, 300 MHz) δ 1.97-2.36 (m, 2H), 3.40-4.10 (m, 4.5H), 4.24 (dd, 0.5H, J=7.6, 11 Hz), 7.10-7.19 (m, 2H), 7.29-7.42 (m, 3H), 7.82 (t, 0.5H, J=1.8 Hz), 7.83 (t, 0.5H, J=1.8 Hz), 8.16 (s, 0.5H), 8.19 (s, 0.5H), 8.53 (s, 0.5H), 8.54 (s, 0.5H), 8.79 (s, 0.5H), 8.81 (s, 0.5H); MS (ESI) m/z=478.1 (MH⁺).

### Example 134

### [3-Chloro-6-(3-fluoro-phenyl)-8-trifluoromethyl-imidazo[1,2-a]pyridin-2-yl]-[3-(4-fluoro-phenyl)-pyrrolidin-1-yl]-methanone (Compound 234)

3-Chloro-6-(3-fluoro-phenyl)-8-trifluoromethyl-imidazo[1,2-a]pyridin-2-yl]-[3-(4-fluoro-phenyl)-pyrrolidin-1-yl]-methanone (compound 234) was prepared in a similar way as for (3-chloro-6-furan-3-yl-8-trifluoromethyl-imidazo[1,2-a]pyridin-2-yl)-[3-(4-fluoro-phenyl)-pyrrolidin-1-yl]-methanone (compound 233). ¹H NMR (d₆-DMSO, 300 MHz) δ 1.99-2.38 (m, 2H), 3.40-4.10 (m, 4.5H), 4.24 (dd, 0.5H, J=7.6, 11.1 Hz), 7.10-7.85 (m, 8H), 8.18 (s, 0.5H), 8.21 (s, 0.5H), 8.86 (s, 0.5H), 8.88 (s, 0.5H); MS (ESI) m/z=506.1 (MH⁺).

### Example 135

### {2-[3-(4-Fluoro-phenyl)-pyrrolidine-1-carbonyl]-6-furan-3-yl-8-trifluoromethyl-imidazo[1,2-a]pyridin-3-yl}-acetic Acid Methyl Ester (Compound 235)

Using standard HATU coupling conditions, 6-furan-3-yl-3-methoxycarbonylmethyl-8-trifluoromethyl-imidazo[1,2-a]pyridine-2-carboxylic acid, and 3-(4-fluorophenyl)pyrrolidine gave {2-[3-(4-fluoro-phenyl)-pyrrolidine-1-carbonyl]-6-furan-3-yl-8-trifluoromethyl-imidazo[1,2-a]pyridin-3-yl}-acetic acid methyl ester (compound 235). ¹H NMR (d₆-DMSO, 300 MHz) δ 1.96-2.40 (m, 2H), 3.40-4.60 (m, 7H), 3.64 (s, 3H), 7.11-7.23 (m, 3H), 7.32-7.41 (m, 2H), 7.82 (t, 0.5H, J=1.8 Hz), 7.82 (t, 0.5H, J=1.8 Hz), 8.10 (s, 0.5H), 8.13 (s, 0.5H), 8.46 (s, 0.5H), 8.47 (s, 0.5H), 8.90 (s, 0.5H), 8.99 (s, 0.5H); MS (ESI) m/z=516.1 (MH⁺).

### Example 136

### {2-[3-(4-Fluoro-phenyl)-pyrrolidine-1-carbonyl]-6-furan-3-yl-8-trifluoromethyl-imidazo[1,2-a]pyridin-3-yl}-acetic Acid (Compound 236)

{2-[3-(4-Fluoro-phenyl)-pyrrolidine-1-carbonyl]-6-furan-3-yl-8-trifluoromethyl-imidazo[1,2-a]pyridin-3-yl}-acetic acid methyl ester was saponified using lithium hydroxide to give {2-[3-(4-fluoro-phenyl)-pyrrolidine-1-carbonyl]-6-furan-3-yl-8-trifluoromethyl-imidazo[1,2-a]pyridin-3-yl}-acetic acid (compound 236). ¹H NMR (d₆-DMSO, 300 MHz) δ 1.92-2.40 (m, 2H), 3.40-4.51 (m, 7H), 7.11-7.24 (m, 3H), 7.32-7.42 (m, 2H), 7.81 (t, 0.5H, J=1.8 Hz), 7.82 (t, 0.5H, J=1.8 Hz), 8.08 (s, 0.5H), 8.10 (s, 0.5H), 8.45 (s, 0.5H), 8.47 (s, 0.5H), 8.96 (brs, 1H), 12.57 (brs, 1H); MS (ESI) m/z=502.1 (MH⁺).

### Example 137

### 2-{2-[3-(4-Fluoro-phenyl)-pyrrolidine-1-carbonyl]-6-furan-3-yl-8-trifluoromethyl-imidazo[1,2-a]pyridin-3-yl}-1-morpholin-4-yl-ethanone (Compound 237)

Using standard HATU coupling conditions, {2-[3-(4-fluoro-phenyl)-pyrrolidine-1-carbonyl]-6-furan-3-yl-8-trifluoromethyl-imidazo[1,2-a]pyridin-3-yl}-acetic acid (compound 236), and morpholine gave 2-{2-[3-(4-fluoro-phenyl)-pyrrolidine-1-carbonyl]-6-furan-3-yl-8-trifluoromethyl-imidazo[1,2-a]pyridin-3-yl}-1-morpholin-4-yl-ethanone (compound 237). MS (ESI) m/z=571.2 (MH⁺).

### Example 138

### [3-Chloro-6-(1H-pyrazol-4-yl)-8-trifluoromethyl-imidazo[1,2-a]pyridin-2-yl]-[3-(4-fluoro-phenyl)-pyrrolidin-1-yl]-methanone (Compound 238)

Under standard HATU coupling conditions, 3-chloro-6-(1H-pyrazol-4-yl)-8-trifluoromethyl-imidazo[1,2-a]pyridine-2-carboxylic acid and 3-(4-fluorophenyl)pyrrolidine gave [3-chloro-6-(1H-pyrazol-4-yl)-8-trifluoromethyl-imidazo[1,2-a]pyridin-2-yl]-[3-(4-fluoro-phenyl)-pyrrolidin-1-yl]-methanone (compound 238). ¹H NMR (d₆-DMSO, 300 MHz) δ 1.96-2.36 (m, 2H), 3.5-4.10 (m, 4.5H), 4.25 (dd, 0.5H, J=7.6, 11.7 Hz), 7.10-7.42 (m, 4H), 8.16 (s, 0.5H), 8.19 (s, 0.5H), 8.38 (s, 1H), 8.39 (s, 1H), 8.81 (s, 0.5H), 8.82 (s, 0.5H); MS (ESI) m/z=478.1 (MH⁺).

### Example 139

### 2-{2-[3-(4-Fluoro-phenyl)-pyrrolidine-1-carbonyl]-6-furan-3-yl-8-trifluoromethyl-imidazo[1,2-a]pyridin-3-yl}-acetamide (Compound 239)

Using standard HATU coupling conditions, {2-[3-(4-fluoro-phenyl)-pyrrolidine-1-carbonyl]-6-furan-3-yl-8-trifluoromethyl-imidazo[1,2-a]pyridin-3-yl}-acetic acid (compound 236), and ammonium chloride gave 2-{2-[3-(4-fluoro-phenyl)-pyrrolidine-1-carbonyl]-6-furan-3-yl-8-trifluoromethyl-imidazo[1,2-a]pyridin-3-yl}-acetamide (compound 239). ¹H NMR (d₆-DMSO, 300 MHz) δ 1.96-2.36 (m, 2H), 3.40-4.44 (m, 7H), 7.06 (brs, 1H), 7.11-7.42 (m, 5H), 7.64 (s, 1H), 7.81 (t, 0.5H, J=1.8 Hz), 7.82 (t, 0.5H, J=1.8 Hz), 8.05 (s, 0.5H), 8.07 (s, 0.5H), 8.43 (s, 0.5H), 8.44 (s, 0.5H), 8.84 (s, 1H); MS (ESI) m/z=501.1 (MH⁺).

### Example 140

### N-Benzyl-2-{2-[3-(4-fluoro-phenyl)-pyrrolidine-1-carbonyl]-6-furan-3-yl-8-trifluoromethyl-imidazo[1,2-a]pyridin-3-yl}-acetamide (Compound 240)

Using standard HATU coupling conditions, {2-[3-(4-fluoro-phenyl)-pyrrolidine-1-carbonyl]-6-furan-3-yl-8-trifluoromethyl-imidazo[1,2-a]pyridin-3-yl}-acetic acid (compound 236), and benzylamine gave N-benzyl-2-{2-[3-(4-fluoro-phenyl)-pyrrolidine-1-carbonyl]-6-furan-3-yl-8-trifluoromethyl-imidazo[1,2-a]pyridin-3-yl}-acetamide (compound 240). ¹H NMR (d₆-DMSO, 300 MHz) δ 1.94-2.40 (m, 2H), 3.36-4.46 (m, 5H), 4.28 (d, 2H, J=5.9 Hz), 4.42 (brs, 2H), 7.10-7.41 (m, 10H), 7.82 (t, 0.5H, J=2 Hz), 7.83 (t, 0.5H, J=2 Hz), 8.05 (s, 0.5H), 8.08 (s, 0.5H), 8.42 (s, 0.5H), 8.43 (s, 0.5H), 8.63 (m, 1H), 8.87 (s, 1H); MS (ESI) m/z=591.2 (MH⁺).

### Example 141

### N-(2-Dimethylamino-ethyl)-2-{2-[3-(4-fluoro-phenyl)-pyrrolidine-1-carbonyl]-6-furan-3-yl-8-trifluoromethyl-imidazo[1,2-a]pyridin-3-yl}-acetamide (Compound 241)

Using standard HATU coupling conditions, {2-[3-(4-fluoro-phenyl)-pyrrolidine-1-carbonyl]-6-furan-3-yl-8-trifluoromethyl-imidazo[1,2-a]pyridin-3-yl}-acetic acid (compound 236), and N,N-dimethylethylenediamine gave N-(2-dimethylamino-ethyl)-2-{2-[3-(4-fluoro-phenyl)-pyrrolidine-1-carbonyl]-6-furan-3-yl-8-trifluoromethyl-imidazo[1,2-a]pyridin-3-yl}-acetamide (compound 241). ¹H NMR (d₆-DMSO, 300 MHz) δ 1.96-2.40 (m, 2H), 2.79 (s, 3H), 2.80 (s, 3H), 4.49 (brs, 2H), 3.12-4.55 (m, 9H), 7.12-7.19 (m, 2H), 7.32-7.42 (m, 3H), 7.80 (t, 0.5H, J=1.8 Hz), 7.81 (t, 0.5H, J=1.8 Hz), 8.07 (s, 0.5H), 8.10 (s, 0.5H), 8.44 (m, 1H), 8.50 (s, 0.5H), 8.51 (s, 0.5H), 9.10 (brs, 1H); MS (ESI) m/z=572.2 (MH⁺).

### Example 142

### N-Cyclopropyl-2-{2-[3-(4-fluoro-phenyl)-pyrrolidine-1-carbonyl]-6-furan-3-yl-8-trifluoromethyl-imidazo[1,2-a]pyridin-3-yl}-acetamide (Compound 242)

Using standard HATU coupling conditions, {2-[3-(4-fluoro-phenyl)-pyrrolidine-1-carbonyl]-6-furan-3-yl-8-trifluoromethyl-imidazo[1,2-a]pyridin-3-yl}-acetic acid (compound 236), and cyclopropylamine gave N-cyclopropyl-2-{2-[3-(4-fluoro-phenyl)-pyrrolidine-1-carbonyl]-6-furan-3-yl-8-trifluoromethyl-imidazo[1,2-a]pyridin-3-yl}-acetamide (compound 242). ¹H NMR (d₆-DMSO, 300 MHz) δ 0.40-0.46 (m, 2H), 0.58-0.65 (m, 2H), 1.96-2.40 (m, 2H), 2.61 (m, 1H), 3.40-4.30 (m, 6.5H), 4.40 (dd, 0.5H, J=7.3, 11.8 Hz), 7.11-7.19 (m, 3H), 7.32-7.41 (m, 2H), 7.82 (t, 0.5H, J=2 Hz), 7.83 (t; 0.5H, J=2 Hz), 8.05 (s, 0.5H), 8.08 (s, 0.5H), 8.27 (s, 0.5H), 8.28 (s, 0.5H), 8.44 (m, 1H), 8.85 (s, 1H); MS (ESI) m/z=541.2 (MH⁺).

### Example 143

### [3-(4-Fluoro-phenyl)-pyrrolidin-1-yl]-[6-furan-3-yl-3-(3-methyl-[1,2,4]oxadiazol-5-ylmethyl)-8-trifluoromethyl-imidazo[1,2-a]pyridin-2-yl]-methanone (Compound 243)

A mixture of {2-[3-(4-fluoro-phenyl)-pyrrolidine-1-carbonyl]-6-furan-3-yl-8-trifluoromethyl-imidazo[1,2-a]pyridin-3-yl}-acetic acid (90 mg, 0.1795 mmol), N-hydroxyacetamide (14.6 mg, 0.1974 mmol), HATU (75.1 mg, 0.1974 mmol) and di-isopropylethylamine (94 L, 0.5384 mmol) was stirred in DMF (1 mL) at room temperature for 145 min. The mixture was diluted with DMF (3 mL) and heated at 120° C. for 15 min under microwave conditions. The mixture was diluted with EtOAc (50 mL) and washed successively with 2N HCl (20 mL), saturated aqueous NaHCO₃ (20 mL), and brine (20 mL). The filtrate was dried (Na₂SO₄), filtered and concentrated. Column chromatography [n-hex/CH₂Cl₂/EtOAc (1:1:2 v/v)] of the crude material gave [3-(4-fluoro-phenyl)-pyrrolidin-1-yl]-[6-furan-3-yl-3-(3-methyl-[1,2,4]oxadiazol-5-ylmethyl)-8-trifluoromethyl-imidazo[1,2-a]pyridin-2-yl]-methanone (50 mg, 52%) as a white powder. ¹H NMR (d₆-DMSO, 300 MHz) δ 90-2.40 (m, 2H), 2.23 (s, 3H), 3.39-4.09 (m, 4H), 4.31 (ddd, 0.5H, J=2.9, 8.5, 11.7 Hz), 4.51 (dd, 0.5H, J=7.0, 11.1 Hz), 5.08-5.12 (m, 2H), 7.11-7.19 (m, 3H), 7.32-7.39 (m, 2H), 7.80 (t, 0.5H, J=2 Hz), 7.81 (t, 0.5H, J=2 Hz), 8.14 (s, 0.5H), 8.17 (s, 0.5H), 8.45 (s, 0.5H), 8.46 (s, 0.5H), 9.04 (s, 0.5H), 9.05 (s, 0.5H); MS (ESI) m/z=540.2 (MH⁺).

### Example 144

### 3-Amino-6-furan-3-yl-8-trifluoromethyl-imidazo[1,2-a]pyridine-2-carboxylic Acid(thiophen-2-ylmethyl)-amide (Compound 244)

A suspension of 6-furan-3-yl-3-nitro-8-trifluoromethyl-imidazo[1,2-a]pyridine-2-carboxylic acid (thiophen-2-ylmethyl)-amide (compound 232) (107.7 mg, 0.2468 mmol), iron powder (82.7 mg, 1.4809 mmol), and ammonium chloride (112.2 mg, 2.0979 mmol) was heated at 100° C. in MeOH (8 mL) and water (1 mL). After 3 hours, the mixture was allowed to stir at room temperature overnight. The mixture was diluted with EtOAc (80 mL) and filtered through a pad of Celite to give a yellow solution. The solution was washed with saturated aqueous NaHCO₃ (20 mL), then brine (20 mL). The filtrate was dried (Na₂SO₄), filtered and concentrated to give a crude solid which was crystallized from CH₂Cl₂/THF to give 3-amino-6-furan-3-yl-8-trifluoromethyl-imidazo[1,2-a]pyridine-2-carboxylic acid (thiophen-2-ylmethyl)-amide (41.7 mg, 42%) as a yellow solid. MS (ESI) m/z=407 (MH⁺).

### Example 145

### 2-{2-[3-(4-Fluoro-phenyl)-pyrrolidine-1-carbonyl]-6-furan-3-yl-8-trifluoromethyl-imidazo[1,2-a]pyridin-3-yl}-N-methyl-acetamide (Compound 245)

Using standard HATU coupling conditions, {2-[3-(4-fluoro-phenyl)-pyrrolidine-1-carbonyl]-6-furan-3-yl-8-trifluoromethyl-imidazo[1,2-a]pyridin-3-yl}-acetic acid (compound 236), and methylamine gave 2-{2-[3-(4-fluoro-phenyl)-pyrrolidine-1-carbonyl]-6-furan-3-yl-8-trifluoromethyl-imidazo[1,2-a]pyridin-3-yl}-N-methyl-acetamide (compound 245). ¹H NMR (d₆-DMSO, 300 MHz) δ 1.94-2.38 (m, 2H), 2.57 (s, 1.5H), 2.60 (s, 1.5H), 3.40-4.34 (m, 6.5H), 4.41 (dd, 0.5H, J=7.5, 11.4 Hz), 7.11-7.18 (m, 3H), 7.30-7.42 (m, 2H), 7.81 (t, 0.5H, J=1.8 Hz), 7.82 (t, 0.5H, J=1.8 Hz), 8.04-8.10 (m, 2H), 8.43 (s, 0.5H), 8.44 (s, 0.5H), 8.87 (s, 1H); MS (ESI) m/z=515.2 (MH⁺).

### Example 146

### (6-Amino-3-chloro-8-trifluoromethyl-imidazo[1,2-a]pyridin-2-yl)-[3-(4-fluoro-phenyl)-pyrrolidin-1-yl]-methanone (Compound 246)

### Step 1: 5-Nitro-3-trifluoromethyl-pyridin-2-ylamine

To a solution of 2-amino-3-(trifluoromethyl)pyridine (2 g, 12.34 mmol) in conc sulfuric acid (10 mL) at 0° C. was added dropwise fuming nitric acid (0.56 mL, 12.34 mmol). After 15 min, the reaction was allowed to stir at room temperature. After 1 hour, the mixture was heated to 50° C. After 2 hours, the reaction was cooled to room temperature and slowly poured into ice-water (200 mL). The precipitate was filtered and dried under high vacuum to give 5-nitro-3-trifluoromethyl-pyridin-2-ylamine (1.92 g, 75%). ¹H NMR (d₆-DMSO, 300 MHz) δ 8.02 (brs, 2H), 8.38 (d, 1H, J=2.6 Hz), 9.04 (d, 1H, J=2.6 Hz); MS (ESI) m/z=208 (MH⁺).

### Step 2: 6-Nitro-8-trifluoromethyl-imidazo[1,2-a]pyridine-2-carboxylic Acid Methyl Ester

Similar to the preparation of 6-bromo-3-methoxycarbonylmethyl-8-trifluoromethyl-imidazo[1,2-a]pyridine-2-carboxylic acid methyl ester (Example 122, step 1), 5-nitro-3-trifluoromethyl-pyridin-2-ylamine (1.295 g, 6.2527 mmol) reacted with methyl bromopyruvate (1.85 mL, 15.632 mmol) in DMF to give 6-nitro-8-trifluoromethyl-imidazo[1,2-a]pyridine-2-carboxylic acid methyl ester (1.71 g, 95%). ¹H NMR (d₆-DMSO, 300 MHz) δ 3.91 (s, 3H), 8.38 (dd, 1H, J=1, 2 Hz), 8.87 (s, 1H), 10.12 (d, 1H, J=2.3 Hz); MS (ESI) m/z=290 (MH⁺).

### Step 3: 3-Chloro-6-nitro-8-trifluoromethyl-imidazo[1,2-a]pyridine-2-carboxylic Acid Methyl Ester

A mixture of 6-nitro-8-trifluoromethyl-imidazo[1,2-a]pyridine-2-carboxylic acid methyl ester (1.71 g, 5.9242 mmol) and N-chlorosuccinimide (831 mg, 6.2204 mmol) was heated at 50° C. in DMF (30 mL) for 3 hours. The mixture was then stirred at room temperature overnight. The mixture was diluted with EtOAc (30 mL) and washed with water (100 mL), 1M sodium thiosulfate solution (100 mL), saturated aqueous NaHCO₃ (100 mL), then brine (100 mL). The filtrate was dried (Na₂SO₄), filtered and concentrated to give 3-chloro-6-nitro-8-trifluoromethyl-imidazo[1,2-a]pyridine-2-carboxylic acid methyl ester (1.856 g, 97%) as a brown solid. ¹H NMR (d₆-DMSO, 300 MHz) δ 3.97 (s, 3H), 8.47 (d, 1H, J=1.8 Hz), 9.57 (d, 1H, J=2 Hz); MS (ESI) m/z=324 (MH⁺).

### Step 4: 6-Amino-3-chloro-8-trifluoromethyl-imidazo[1,2-a]pyridine-2-carboxylic Acid Methyl Ester

A suspension of 3-chloro-6-nitro-8-trifluoromethyl-imidazo[1,2-a]pyridine-2-carboxylic acid methyl ester (487 mg, 1.5049 mmol), and Raney®-nickel (0.5 mL) in acetic acid (0.5 mL) and MeOH (50 mL) was shaken under hydrogen at 40 psi for 7 hours. The catalyst was filtered and the solvent was concentrated under reduced pressure. The crude material was absorbed on silica gel and chromatographed [CH₂Cl₂/MeOH (98:2 v/v) to (97:3 v/v)] to give 6-amino-3-chloro-8-trifluoromethyl-imidazo[1,2-a]pyridine-2-carboxylic acid methyl ester (400 mg, 91%) as a brown solid. ¹H NMR (d₆-DMSO, 300 MHz) δ 3.86 (s, 3H), 5.67 (s, 2H), 7.56 (m, 1H), 7.71 (m, 1H); MS (ESI) m/z=294 (MH⁺).

### Step 5: 6-Amino-3-chloro-8-trifluoromethyl-imidazo[1,2-a]pyridine-2-carboxylic Acid

To a stirred solution of 6-amino-3-chloro-8-trifluoromethyl-imidazo[1,2-a]pyridine-2-carboxylic acid methyl ester (100 mg, 0.3406 mmol) in THF (9 mL) was added a solution of lithium hydroxide monohydrate (28.6 mg, 0.6811 mmol) in water (3 mL). After 4.5 hours, the solvent was concentrated followed by the addition of 2N HCl (1.2 mL). The aqueous solution was extracted with EtOAc (20 mL, 10 mL), and the extracts were dried (Na₂SO₄), filtered and concentrated to give 6-amino-3-chloro-8-trifluoromethyl-imidazo[1,2-a]pyridine-2-carboxylic acid (95 mg, 100%) as a brown solid. ¹H NMR (d₆-DMSO, 300 MHz) δ 7.54 (s, 1H), 7.70 (s, 1H); MS (ESI) m/z=280 (MH⁺).

### Step 6: (6-Amino-3-chloro-8-trifluoromethyl-imidazo[1,2-a]pyridin-2-yl)-[3-(4-fluoro-phenyl)-pyrrolidin-1-yl]-methanone (compound 246)

Under standard HATU coupling conditions, 6-amino-3-chloro-8-trifluoromethyl-imidazo[1,2-a]pyridine-2-carboxylic acid and 3-(4-fluoro-phenyl)-pyrrolidine gave (6-amino-3-chloro-8-trifluoromethyl-imidazo[1,2-a]pyridin-2-yl)-[3-(4-fluoro-phenyl)-pyrrolidin-1-yl]-methanone. ¹H NMR (d₆-DMSO, 300 MHz) δ 1.96-2.34 (m, 2H), 3.36-4.12 (m, 4.5H), 4.27 (dd, 0.5H, J=7.6, 10.8 Hz), 5.59 (d, 2H, J=5.2 Hz), 7.10-7.18 (m, 2H), 7.32-7.42 (m, 2H), 7.50 (m, 1H), 7.72 (m, 1H); MS (ESI) m/z=429 (MH⁺).

### Example 147

### N-{3-Chloro-2-[3-(4-fluoro-phenyl)-pyrrolidine-1-carbonyl]-8-trifluoromethyl-imidazo[1,2-a]pyridin-6-yl}-acetamide (Compound 247)

To a solution of (6-amino-3-chloro-8-trifluoromethyl-imidazo[1,2-a]pyridin-2-yl)-[3-(4-fluoro-phenyl)-pyrrolidin-1-yl]-methanone (30 mg, 0.0703 mmol) in DMF (1 mL) was added pyridine (28.4 μL, 0.3515 mmol) and acetyl chloride (7.5 μL, 0.1054 mmol). After 4 hours, the mixture was diluted with EtOAc (20 mL) and washed with brine (2×10 mL). The extracts were dried (Na₂SO₄), filtered and concentrated. Column chromatography [EtOAc/n-hex (3:1 v/v) to (5:1 v/v) then EtOAc] of the crude material gave N-{3-chloro-2-[3-(4-fluoro-phenyl)-pyrrolidine-1-carbonyl]-8-trifluoromethyl-imidazo[1,2-a]pyridin-6-yl}-acetamide (17.2 mg, 52%) as a white powder. ¹H NMR (d₆-DMSO, 300 MHz) δ 1.96-2.34 (m, 2H), 2.13 (s, 1.5H), 2.14 (s, 1.5H), 3.40-4.10 (m, 4.5H), 4.24 (dd, 0.5H, J=7.3, 10.8 Hz), 7.10-7.20 (m, 2H), 7.30-7.42 (m, 2H), 7.80 (brs, 0.5H), 7.83 (brs, 0.5H), 9.23 (brs, 0.5H), 9.24 (brs, 0.5H), 10.46 (s, 0.5H), 10.48 (s, 0.5H); MS (ESI) m/z=469.1 (MH⁺).

### Example 148

### 6-Phenyl-8-trifluoromethyl-imidazo[1,2-b]pyridazine-2-carboxylic Acid (thiophen-2-ylmethyl)-amide (Compound 248)

### Step 1: 6-Phenyl-4-trifluoromethyl-pyridazin-3-ylamine

A mixture of 3-chloro-6-phenyl-4-trifluoromethyl-pyridazine (0.79 g, 3.05 mmol) was heated in 2N ammonia in iso-propanol (60 mL) at 100° C. in a sealed tube for 3 days. Additional 2N ammonia in iso-propanol (10 mL) was added to the reaction and heated for 1 day. Upon cooling, the solvent was removed under reduced pressure. The solid was digested with THF (25 mL) and the undissolved solid was filtered. Concentration of the filtrate gave 6-phenyl-4-trifluoromethyl-pyridazin-3-ylamine (749.8 mg, quantitative) as a light yellow solid. ¹H NMR (d₆-DMSO, 300 MHz) δ 7.11 (s, 2H), 7.39-7.51 (m, 3H), 8.02-8.07 (m, 3H); MS (ESI) m/z=240.1 (MH⁺).

### Step 2: 6-Phenyl-8-trifluoromethyl-imidazo[1,2-b]pyridazine-2-carboxylic Acid Methyl Ester

Similar to the preparation of 6-bromo-3-methoxycarbonylmethyl-8-trifluoromethyl-imidazo[1,2-a]pyridine-2-carboxylic acid methyl ester (Example 122, step 1), 6-phenyl-4-trifluoromethyl-pyridazin-3-ylamine (745 mg, 3.1145 mmol) reacted with methyl bromopyruvate (0.92 mL, 7.7864 mmol) in DMF (15 mL) to give 6-phenyl-8-trifluoromethyl-imidazo[1,2-b]pyridazine-2-carboxylic acid methyl ester (701.2 mg, 70%) as a white solid. ¹H NMR (d₆-DMSO, 300 MHz) δ 3.90 (s, 3H), 7.58-7.62 (m, 3H), 8.13-8.20 (m, 2H), 8.31 (d, H, J=0.8 Hz), 9.11 (s, 1H); MS (ESI) m/z=322.1 (MH⁺).

### Step 3: 6-Phenyl-8-trifluoromethyl-imidazo[1,2-b]pyridazine-2-carboxylic Acid

6-Phenyl-8-trifluoromethyl-imidazo[1,2-b]pyridazine-2-carboxylic acid methyl ester was saponified using a similar method as for the preparation of 6-amino-3-chloro-8-trifluoromethyl-imidazo[1,2-a]pyridine-2-carboxylic acid (Example 146, step 5) to give 6-phenyl-8-trifluoromethyl-imidazo[1,2-b]pyridazine-2-carboxylic acid as a beige colored solid. ¹H NMR (d₆-DMSO, 300 MHz) δ 7.56-7.62 (m, 3H), 8.13-8.20 (m, 2H), 8.28 (d, H, J=1.1 Hz), 9.00 (s, 1H), 13.18 (brs, 1H); MS (ESI) m/z=308 (MH⁺).

### Step 4: 6-Phenyl-8-trifluoromethyl-imidazo[1,2-b]pyridazine-2-carboxylic Acid (thiophen-2-ylmethyl)-amide (compound 248)

Under standard HATU coupling conditions, 6-phenyl-8-trifluoromethyl-imidazo[1,2-b]pyridazine-2-carboxylic acid and thiophene-2-methylamine gave 6-phenyl-8-trifluoromethyl-imidazo[1,2-b]pyridazine-2-carboxylic acid (thiophen-2-ylmethyl)-amide. ¹H NMR (d₆-DMSO, 300 MHz) δ 4.66 (d, 2H, J=6.2 Hz), 6.95 (dd, 1H, J=3.5, 5 Hz), 7.03 (dd, 1H, J=1.2, 3.2 Hz), 7.37 (dd, 1H, J=1.2, 5 Hz), 7.56-7.62 (m, 3H), 8.14-8.20 (m, 2H), 8.29 (s, 1H), 8.91 (s, 1H), 8.99 (t, 1H, J=6.2 Hz); MS (ESI) m/z=403 (MH⁺).

### Example 149

### 3-Chloro-6-phenyl-8-trifluoromethyl-imidazo[1,2-b]pyridazine-2-carboxylic Acid(thiophen-2-ylmethyl)-amide (Compound 249)

### Step 1: 3-Chloro-6-phenyl-8-trifluoromethyl-imidazo[1,2-b]pyridazine-2-carboxylic Acid Methyl Ester

Using similar procedure as for the preparation of 3-chloro-6-nitro-8-trifluoromethyl-imidazo[1,2-a]pyridine-2-carboxylic acid methyl ester (Example 146, Step 3) 6-phenyl-8-trifluoromethyl-imidazo[1,2-b]pyridazine-2-carboxylic acid methyl ester reacted with N-chlorosuccinimide to give 3-chloro-6-phenyl-8-trifluoromethyl-imidazo[1,2-b]pyridazine-2-carboxylic acid methyl ester. ¹H NMR (d₆-DMSO, 300 MHz) δ 3.93 (s, 3H), 7.60-7.64 (m, 3H), 8.20-8.24 (m, 2H), 8.41 (d, 1H, J=1.2 Hz); MS (ESI) m/z=356 (MH⁺)

### Step 2: 3-Chloro-6-phenyl-8-trifluoromethyl-imidazo[1,2-b]pyridazine-2-carboxylic Acid

3-Chloro-6-phenyl-8-trifluoromethyl-imidazo[1,2-b]pyridazine-2-carboxylic acid methyl ester was saponified using a similar method as for the preparation of 6-amino-3-chloro-8-trifluoromethyl-imidazo[1,2-a]pyridine-2-carboxylic acid (Example 146, step 5) to give 3-chloro-6-phenyl-8-trifluoromethyl-imidazo[1,2-b]pyridazine-2-carboxylic acid as an off-white solid. ¹H NMR (d₆-DMSO, 300 MHz) [symbol] 7.58-7.64 (m, 3H), 8.20-8.25 (m, 2H), 8.39 (d, H, J=1.2 Hz); MS (ESI) m/z=342 (MH⁺).

### Step 3: 3-Chloro-6-phenyl-8-trifluoromethyl-imidazo[1,2-b]pyridazine-2-carboxylic Acid(thiophen-2-ylmethyl)-amide (compound 249)

Under standard HATU coupling conditions, 3-chloro-6-phenyl-8-trifluoromethyl-imidazo[1,2-b]pyridazine-2-carboxylic acid and thiophene-2-methylamine gave 3-chloro-6-phenyl-8-trifluoromethyl-imidazo[1,2-b]pyridazine-2-carboxylic acid (thiophen-2-ylmethyl)-amide. ¹H NMR (d₆-DMSO, 300 MHz) δ 4.65 (d, 2H, J=6.2 Hz), 6.96 (dd, 1H, J=3.2, 5 Hz), 7.04 (dd, 1H, J=1.2, 3.5 Hz), 7.38 (dd, 1H, J=1.2, 5 Hz), 7.58-7.64 (m, 3H), 8.18-8.26 (m, 2H), 8.39 (d, 1H, J=1.2 Hz), 9.03 (t, 1H, J=6.2 Hz); MS (ESI) m/z=437 (MH⁺).

### Example 150

### 3-Bromo-6-phenyl-8-trifluoromethyl-imidazo[1,2-b]pyridazine-2-carboxylic Acid(thiophen-2-ylmethyl)-amide (Compound 250)

### Step 1: 3-Bromo-6-phenyl-8-trifluoromethyl-imidazo[1,2-b]pyridazine-2-carboxylic Acid Methyl Ester

Using similar procedure as for the preparation of 3-chloro-6-nitro-8-trifluoromethyl-imidazo[1,2-a]pyridine-2-carboxylic acid methyl ester (Example 146, Step 3) 6-phenyl-8-trifluoromethyl-imidazo[1,2-b]pyridazine-2-carboxylic acid methyl ester reacted with N-bromosuccinimide to give 3-bromo-6-phenyl-8-trifluoromethyl-imidazo[1,2-b]pyridazine-2-carboxylic acid methyl ester. ¹H NMR (d₆-DMSO, 300 MHz) δ 3.93 (s, 3H), 7.60-7.66 (m, 3H), 8.20-8.26 (m, 2H), 8.41 (s, 1H); MS (ESI) m/z=399.9 (MH⁺).

### Step 2: 3-Bromo-6-phenyl-8-trifluoromethyl-imidazo[1,2-b]pyridazine-2-carboxylic Acid

3-Bromo-6-phenyl-8-trifluoromethyl-imidazo[1,2-b]pyridazine-2-carboxylic acid methyl ester was saponified using a similar method as for the preparation of 6-amino-3-chloro-8-trifluoromethyl-imidazo[1,2-a]pyridine-2-carboxylic acid (Example 146, step 5) to give 3-bromo-6-phenyl-8-trifluoromethyl-imidazo[1,2-b]pyridazine-2-carboxylic acid as a white solid. ¹H NMR (d₆-DMSO, 300 MHz) δ 7.59-7.65 (m, 3H), 8.20-8.26 (m, 2H), 8.39 (d, H, J=0.9 Hz); MS (ESI) m/z=388 (MH⁺).

### Step 3: 3-Bromo-6-phenyl-8-trifluoromethyl-imidazo[1,2-b]pyridazine-2-carboxylic Acid(thiophen-2-ylmethyl)-amide (Compound 250)

Under standard HATU coupling conditions, 3-bromo-6-phenyl-8-trifluoromethyl-imidazo[1,2-b]pyridazine-2-carboxylic acid and thiophene-2-methylamine gave 3-bromo-6-phenyl-8-trifluoromethyl-imidazo[1,2-b]pyridazine-2-carboxylic acid (thiophen-2-ylmethyl)-amide. ¹H NMR (d₆-DMSO, 300 MHz) δ 4.65 (d, 2H, J=6.2 Hz), 6.96 (dd, 1H, J=0.32, 5 Hz), 7.04 (dd, 1H, J=1.5, 3.5 Hz), 7.38 (dd, 1H, J=1.5, 5 Hz), 7.58-7.64 (m, 3H), 8.18-8.26 (m, 2H), 8.39 (d, 1H, J=0.9 Hz), 9.01 (t, 1H, J=6.2 Hz); MS (ESI) m/z=483 (MH⁺).

### Example 151

### [3-(4-Fluoro-phenyl)-pyrrolidin-1-yl]-(6-furan-3-yl-8-trifluoromethyl-imidazo[1,2-a]pyridin-2-yl)-methanone (Compound 251)

### Step 1: 6-Furan-3-yl-8-trifluoromethyl-imidazo[1,2-a]pyridine-2-carboxylic Acid

A mixture of 6-bromo-8-trifluoromethyl-imidazo[1,2-a]pyridine-2-carboxylic acid methyl ester (600 mg, 1.8572 mmol), furan-3-boronic acid (291 mg, 2.60 mmol), tetrakis(triphenylphosphine)palladium(0) (107 mg, 0.0928 mmol) in 1M K₃PO₄ (2.5 mL) and 1,4-dioxane (12.5 mL) was heated at 90° C. for 135 min. The mixture was diluted with EtOAc (120 mL) and washed with saturated aqueous NaHCO₃ (20 mL), and brine (20 mL). The solution was diluted with n-hex (50 mL) and loaded on a pad of silica gel which was eluted with EtOAc/n-hex (2:1 v/v) to give 6-furan-3-yl-8-trifluoromethyl-imidazo[1,2-a]pyridine-2-carboxylic acid methyl ester (653.7 mg) as a light brown solid. The partially purified methyl ester was dissolved in THF (90 mL) and treated with lithium hydroxide monohydrate (220 mg, 5.238 mmol) in water (30 mL). After 4.5 hours, the solvent was removed under reduced pressure, diluted with 10% NaOH (20 mL) and washed with Et₂O (100 mL). The aqueous phase was acidified with 6N HCl, extracted with EtOAc (2×100 mL). The filtrate was dried (Na₂SO₄), filtered and concentrated to give 6-furan-3-yl-8-trifluoromethyl-imidazo[1,2-a]pyridine-2-carboxylic acid (520 mg, 84%) a light yellow solid. ¹H NMR (d₆-DMSO, 300 MHz) δ 7.01 (dd, 1H, J=0.8, 1.7 Hz), 7.83 (t, 1H, J=1.7 Hz), 8.11 (brs, 1H), 8.44 (brs, 1H), 8.51 (s, 1H), 9.11 (s, 1H), 13.00 (brs, 1H); MS (ESI) m/z=297 (MH⁺).

### Step 2: [3-(4-Fluoro-phenyl)-pyrrolidin-1-yl]-(6-furan-3-yl-8-trifluoromethyl-imidazo[1,2-a]pyridin-2-yl)-methanone (Compound 251)

Under standard HATU coupling conditions, 6-furan-3-yl-8-trifluoromethyl-imidazo[1,2-a]pyridine-2-carboxylic acid and 3-(4-fluorophenyl)pyrrolidine gave [3-(4-fluoro-phenyl)-pyrrolidin-1-yl]-(6-furan-3-yl-8-trifluoromethyl-imidazo[1,2-a]pyridin-2-yl)-methanone. ¹H NMR (d₆-DMSO, 300 MHz) δ 1.96-2.40 (m, 2H), 3.40-4.37 (m, 4.5H), 4.53 (dd, 0.5H, J=7, 10.5 Hz), 7.01 (dd, 0.5H, J=0.9, 2 Hz), 7.02 (dd, 0.5H, J=0.9, 2 Hz), 7.16 (room temperature, 2H, J=9 Hz), 7.32-7.42 (m, 2H), 7.82 (t, 0.5H, J=1.8 Hz), 7.83 (t, 0.5H, J=1.8 Hz), 8.07 (s, 0.5H), 8.09 (s, 0.5H), 8.41-8.45 (m, 2H), 9.12 (s, 0.5H), 9.14 (s, 0.5H); MS (ESI) m/z=444 (MH⁺).

### Example 152

### (3-Bromo-6-phenyl-8-trifluoromethyl-imidazo[1,2-b]pyridazin-2-yl)-[3-(4-fluoro-phenyl)-pyrrolidin-1-yl]-methanone (Compound 252)

Under standard HATU coupling conditions, 3-bromo-6-phenyl-8-trifluoromethyl-imidazo[1,2-b]pyridazine-2-carboxylic acid and 3-(4-fluorophenyl)pyrrolidine gave (3-bromo-6-phenyl-8-trifluoromethyl-imidazo[1,2-b]pyridazin-2-yl)-[3-(4-fluoro-phenyl)-pyrrolidin-1-yl]-methanone. ¹H NMR (d₆-DMSO, 300 MHz) δ 2.00-2.36 (m, 2H), 3.40-4.10 (m, 4.5H), 4.17 (dd, 0.5H, J=7.3, 10.8 Hz), 7.20-7.20 (m, 2H), 7.32-7.44 (m, 2H), 7.58-7.64 (m, 3H), 8.17-8.25 (m, 2H), 8.34 (d, 0.5H, J=0.9 Hz), 8.37 (d, 0.5H, J=0.9 Hz); MS (ESI) m/z=535 (MH⁺).

### Example 153

### (3-Bromo-6-furan-3-yl-8-trifluoromethyl-imidazo[1,2-a]pyridin-2-yl)-[3-(4-fluoro-phenyl)-pyrrolidin-1-yl]-methanone (Compound 253)

### Step 1: 3,6-Dibromo-8-trifluoromethyl-imidazo[1,2-a]pyridine-2-carboxylic Acid Methyl Ester

Using similar procedure as for the preparation of 3-chloro-6-nitro-8-trifluoromethyl-imidazo[1,2-a]pyridine-2-carboxylic acid methyl ester (Example 146, Step 3) 6-bromo-8-trifluoromethyl-imidazo[1,2-a]pyridine-2-carboxylic acid methyl ester reacted with N-bromosuccinimide to give 3,6-dibromo-8-trifluoromethyl-imidazo[1,2-a]pyridine-2-carboxylic acid methyl ester. ¹H NMR (d₆-DMSO, 300 MHz) δ 3.89 (s, 3H), 8.12 (m, 1H), 8.92 (m, 1H); MS (ESI) m/z=400.9, 402.9 (MH⁺).

### Step 2: 3,6-Dibromo-8-trifluoromethyl-imidazo[1,2-a]pyridine-2-carboxylic Acid

3,6-Dibromo-8-trifluoromethyl-imidazo[1,2-a]pyridine-2-carboxylic acid methyl ester was saponified using a similar method as for the preparation of 6-amino-3-chloro-8-trifluoromethyl-imidazo[1,2-a]pyridine-2-carboxylic acid (Example 146, step 5) to give 3,6-dibromo-8-trifluoromethyl-imidazo[1,2-a]pyridine-2-carboxylic acid. MS (ESI) m/z=388.9 (MH⁺).

### Step 3: (3,6-Dibromo-8-trifluoromethyl-imidazo[1,2-a]pyridin-2-yl)-[3-(4-fluoro-phenyl)-pyrrolidin-1-yl]-methanone

Under standard HATU coupling conditions, 3,6-dibromo-8-trifluoromethyl-imidazo[1,2-a]pyridine-2-carboxylic acid and 3-(4-fluorophenyl)pyrrolidine gave (3,6-dibromo-8-trifluoromethyl-imidazo[1,2-a]pyridin-2-yl)-[3-(4-fluoro-phenyl)-pyrrolidin-1-yl]-methanone. ¹H NMR (d₆-DMSO, 300 MHz) δ 1.96-2.34 (m, 2H), 3.40-4.08 (m, 4.5H), 4.12 (dd, 0.5H, J=6.7, 11.1 Hz), 7.09-7.20 (m, 2H), 7.30-7.42 (m, 2H), 8.05 (s, 0.5H), 8.08 (s, 0.5H), 8.88 (s, 0.5H), 8.90 (s, 0.5H); MS (ESI) m/z=537.9 (MH⁺).

### Step 4: (3-Bromo-6-furan-3-yl-8-trifluoromethyl-imidazo[1,2-a]pyridin-2-yl)-[3-(4-fluoro-phenyl)-pyrrolidin-1-yl]-methanone (Compound 253)

A mixture of (3,6-dibromo-8-trifluoromethyl-imidazo[1,2-a]pyridin-2-yl)-[3-(4-fluoro-phenyl)-pyrrolidin-1-yl]-methanone (100 mg, 0.1869 mmol), furan-3-boronic acid (31.4 mg, 0.2803 mmol), tetrakis(triphenylphosphine)palladium(0) (10.8 mg, 0.0093 mmol) in 1M K₃PO₄ (0.3 mL) and 1,4-dioxane (1.2 mL) was heated at 80° C. for 10 min under microwave conditions. The mixture was diluted with EtOAc (40 mL) and washed with saturated aqueous NaHCO₃ (10 mL), and brine (10 mL). The extracts were dried (Na₂SO₄), filtered and concentrated. Column chromatography [n-hex/EtOAc (3:2 v/v)] and [CH₂Cl₂/ACN (12:1 v/v)] of the crude material gave (3-bromo-6-furan-3-yl-8-trifluoromethyl-imidazo[1,2-a]pyridin-2-yl)-[3-(4-fluoro-phenyl)-pyrrolidin-1-yl]-methanone (28.7 mg, 29%) as a white powder. ¹H NMR (d₆-DMSO, 300 MHz) δ 1.96-2.36 (m, 2H), 3.40-4.10 (m, 4.5H), 4.18 (dd, 0.5H, J=7.3, 10.8 Hz), 7.10-7.20 (m, 2H), 7.27-7.43 (m, 3H), 7.82 (t, 0.5H, J=1.8 Hz), 7.83 (t, 0.5H, J=1.8 Hz), 8.16 (s, 0.5H), 8.19 (s, 0.5H), 8.53 (s, 0.5H), 8.55 (s, 0.5H), 8.72 (s, 0.5H), 8.73 (s, 0.5H); MS (ESI) m/z=522 (MH⁺).

### Example 154

### (3,6-Di-furan-3-yl-8-trifluoromethyl-imidazo[1,2-a]pyridin-2-yl)-[3-(4-fluoro-phenyl)-pyrrolidin-1-yl]-methanone (Compound 254)

A mixture of (3,6-dibromo-8-trifluoromethyl-imidazo[1,2-a]pyridin-2-yl)-[3-(4-fluoro-phenyl)-pyrrolidin-1-yl]-methanone (50 mg, 0.0934 mmol), furan-3-boronic acid (52.3 mg, 0.4672 mmol), tetrakis(triphenylphosphine)palladium(0) (5.4 mg, 0.0047 mmol) in 1M K₃PO₄ (0.3 mL) and 1,4-dioxane (0.9 mL) was heated at 120° C. for 10 min under microwave conditions. The mixture was diluted with EtOAc (40 mL) and washed with saturated aqueous NaHCO₃ (10 mL), and brine (10 mL). The extracts were dried (Na₂SO₄), filtered and concentrated. Column chromatography [CH₂Cl₂/ACN (10:1 v/v)] of the crude material gave (3,6-di-furan-3-yl-8-trifluoromethyl-imidazo[1,2-a]pyridin-2-yl)-[3-(4-fluoro-phenyl)-pyrrolidin-1-yl]-methanone (34.6 mg, 73%) as a white powder. ¹H NMR (d₆-DMSO, 300 MHz) δ 1.94-2.32 (m, 2H), 3.36-4.04 (m, 5H), 6.96 (dd, 0.5H, J=0.9, 1.8 Hz), 6.97 (dd, 0.5H, J=0.9, 1.8 Hz), 7.06-7.18 (m, 3H), 7.24-7.39 (m, 2H), 7.79 (t, 0.5H, J=1.8 Hz), 7.80 (t, 0.5H, J=1.8 Hz), 7.91 (t, 0.5H, J=1.5 Hz), 7.79 (t, 0.5H, J=1.5 Hz), 8.09 (s, 0.5H), 8.11 (s, 0.5H), 8.32 (dd, 0.5H, J=0.9, 1.5 Hz), 8.33 (dd, 0.5H, J=0.9, 1.5 Hz), 8.45 (brs, 0.5H), 8.47 (brs, 0.5H), 8.61 (s, 0.5H), 8.62 (s, 0.5H); MS (ESI) m/z=510.1 (MH⁺).

### Example 155

### [3-(4-Fluoro-phenyl)-pyrrolidin-1-yl]-[6-(1H-pyrazol-4-yl)-8-trifluoromethyl-imidazo[1,2-a]pyridin-2-yl]-methanone (Compound 255)

### Step 1: 6-Bromo-8-trifluoromethyl-imidazo[1,2-a]pyridine-2-carboxylic Acid

Using similar method as for the preparation of 6-bromo-3-chloro-8-trifluoromethyl-imidazo[1,2-a]pyridine-2-carboxylic acid (Example 133, Step 1), 6-bromo-8-trifluoromethyl-imidazo[1,2-a]pyridine-2-carboxylic acid ethyl ester was treated with hydrochloric acid to give 6-bromo-8-trifluoromethyl-imidazo[1,2-a]pyridine-2-carboxylic acid. ¹H NMR (d₆-DMSO, 300 MHz) δ 7.97 (m, 1H), 8.53 (s, 1H), 9.17 (m, 1H), 13.11 (brs, 1H); MS (ESI) m/z=310.9 (MH⁺).

### Step 2: (6-Bromo-8-trifluoromethyl-imidazo[1,2-a]pyridin-2-yl)-[3-(4-fluoro-phenyl)-pyrrolidin-1-yl]-methanone

Under standard HATU coupling conditions, 6-bromo-8-trifluoromethyl-imidazo[1,2-a]pyridine-2-carboxylic acid and 3-(4-fluorophenyl)pyrrolidine gave (6-bromo-8-trifluoromethyl-imidazo[1,2-a]pyridin-2-yl)-[3-(4-fluoro-phenyl)-pyrrolidin-1-yl]-methanone. ¹H NMR (d₆-DMSO, 300 MHz) δ 1.96-2.40 (m, 2H), 3.40-4.36 (m, 4.5H), 4.50 (dd, 0.5H, J=7.6, 11.1 Hz), 7.15 (dt, 2H, J=0.8, 8.8 Hz), 7.32-7.41 (m, 2H), 7.93 (m, 0.5H), 7.96 (m, 0.5H), 8.45 (s, 0.5H), 8.46 (s, 0.5H), 9.17 (m, 0.5H), 9.19 (m, 0.5H); MS (ESI) m/z=458 (MH⁺).

### Step 3: [3-(4-Fluoro-phenyl)-pyrrolidin-1-yl]-[6-(1H-pyrazol-4-yl)-8-trifluoromethyl-imidazo[1,2-a]pyridin-2-yl]-methanone (compound 255)

A mixture of (6-bromo-8-trifluoromethyl-imidazo[1,2-a]pyridin-2-yl)-[3-(4-fluoro-phenyl)-pyrrolidin-1-yl]-methanone (50 mg, 0.1096 mmol), 4-pyrazoleboronic acid pinacol ester (74.4 mg, 0.3836 mmol), tetrakis(triphenylphosphine)palladium(0) (6.3 mg, 0.0055 mmol) in 1M K₃PO₄ (0.4 mL) and 1,4-dioxane (1.2 mL) was heated at 140° C. for 25 min under microwave conditions. The mixture was diluted with EtOAc (25 mL) and washed with saturated aqueous NaHCO₃ (10 mL), and brine (10 mL). The extracts were dried (Na₂SO₄), filtered and concentrated. Preparative HPLC purification (30-100% ACN gradient) of the crude product gave [3-(4-fluoro-phenyl)-pyrrolidin-1-yl]-[6-(1H-pyrazol-4-yl)-8-trifluoromethyl-imidazo[1,2-a]pyridin-2-yl]-methanone (12.3 mg, 25%) as a white powder. ¹H NMR (d₆-DMSO, 300 MHz) δ 1.92-2.40 (m, 2H), 3.40-4.40 (m, 4.5H), 4.54 (dd, 0.5H, J=7.6, 11.7 Hz), 7.15 (brt, 2H, J=9.1 Hz), 7.34-7.42 (m, 2H), 8.02 (brs, 1H), 8.04 (s, 0.5H), 8.07 (s, 0.5H), 8.38 (brs, 1H), 8.40 (s, 0.5H), 8.41 (s, 0.5H), 9.10 (s, 0.5H), 9.12 (s, 0.5H), 13.10 (brs, 1H); MS (ESI) m/z=444.1 (MH⁺).

### Example 156

### [3-Bromo-6-(1H-pyrazol-4-yl)-8-trifluoromethyl-imidazo[1,2-a]pyridin-2-yl]-[3-(4-fluoro-phenyl)-pyrrolidin-1-yl]-methanone (Compound 256)

Similar to the preparation of (3-bromo-6-furan-3-yl-8-trifluoromethyl-imidazo[1,2-a]pyridin-2-yl)-[3-(4-fluoro-phenyl)-pyrrolidin-1-yl]-methanone (compound 253), (3,6-dibromo-8-trifluoromethyl-imidazo[1,2-a]pyridin-2-yl)-[3-(4-fluoro-phenyl)-pyrrolidin-1-yl]-methanone and 4-pyrazoleboronic acid pinacol ester reacted under microwave conditions to give [3-bromo-6-(1H-pyrazol-4-yl)-8-trifluoromethyl-imidazo[1,2-a]pyridin-2-yl]-[3-(4-fluoro-phenyl)-pyrrolidin-1-yl]-methanone. ¹H NMR (d₆-DMSO, 300 MHz) δ 1.96-2.36 (m, 2H), 3.40-4.08 (m, 4.5H), 4.18 (dd, 0.5H, J=7.6, 11.4 Hz), 7.10-7.20 (m, 2H), 7.30-7.43 (m, 2H), 8.16 (s, 0.5H), 8.19 (s, 0.5H), 8.22 (brs, 1H), 8.54 (brs, 1H), 8.73 (s, 0.5H), 8.75 (s, 0.5H), 13.14 (s, 1H); MS (ESI) m/z=523.1 (MH⁺).

### Example 157

### [3-Chloro-6-(1H-pyrazol-4-yl)-8-trifluoromethyl-imidazo[1,2-a]pyridin-2-yl]-[3-(2-fluoro-phenyl)-pyrrolidin-1-yl]-methanone (Compound 257)

### Step 1: 3-Chloro-6-(1H-pyrazol-4-yl)-8-trifluoromethyl-imidazo[1,2-a]pyridine-2-carboxylic Acid

A mixture of 6-bromo-3-chloro-8-trifluoromethyl-imidazo[1,2-a]pyridine-2-carboxylic acid ethyl ester (371.5 mg, 1 mmol), 4-pyrazoleboronic acid pinacol ester (582.1 mg, 3 mmol), tetrakis(triphenylphosphine)palladium(0) (57.8 mg, 0.05 mmol) in 1M K₃PO₄ (3 mL) and 1,4-dioxane (12 mL) was heated at 140° C. for 15 min under microwave conditions. Additional 1M K₃PO₄ (5 mL) was added to the reaction mixture and heated again at 120° C. for min under microwave conditions. The solvent was removed under reduced pressure, 10% citric acid (20 mL) was added followed by extraction with EtOAc (2×100 mL, 50 mL). The extracts were dried (Na₂SO₄), filtered and concentrated. Column chromatography [CH₂Cl₂/MeOH/AcOH (8:1:0.1 v/v) to (4:1:0.1 v/v)] of the crude material gave 3-chloro-6-(1H-pyrazol-4-yl)-8-trifluoromethyl-imidazo[1,2-a]pyridine-2-carboxylic acid (70.1 mg, 21%) as a grey powder.

### Step 2: [3-Chloro-6-(1H-pyrazol-4-yl)-8-trifluoromethyl-imidazo[1,2-a]pyridin-2-yl]-[3-(2-fluoro-phenyl)-pyrrolidin-1-yl]-methanone (compound 257)

Under standard HATU coupling conditions, 3-chloro-6-(1H-pyrazol-4-yl)-8-trifluoromethyl-imidazo[1,2-a]pyridine-2-carboxylic acid and 3-(2-fluorophenyl)pyrrolidine gave [3-chloro-6-(1H-pyrazol-4-yl)-8-trifluoromethyl-imidazo[1,2-a]pyridin-2-yl]-[3-(2-fluoro-phenyl)-pyrrolidin-1-yl]-methanone. ¹H NMR (d₆-DMSO, 300 MHz) δ 2.03-2.36 (m, 2H), 3.48-4.14 (m, 4.5H), 4.29 (dd, 0.5H, J=6.7, 10.5 Hz), 7.12-7.46 (m, 4H), 8.16 (s, 0.5H), 8.19 (s, 0.5H), 8.23 (brs, 1H), 8.54 (brs, 1H), 8.81 (s, 0.5H), 8.82 (s, 0.5H), 13.13 (brs, 1H); MS (ESI) m/z=478.1 (MH⁺).

### Example 158

### [3-Chloro-6-(1H-pyrazol-4-yl)-8-trifluoromethyl-imidazo[1,2-a]pyridin-2-yl]-[3-(3-fluoro-phenyl)-pyrrolidin-1-yl]-methanone (Compound 258)

[3-Chloro-6-(1H-pyrazol-4-yl)-8-trifluoromethyl-imidazo[1,2-a]pyridin-2-yl]-[3-(3-fluoro-phenyl)-pyrrolidin-1-yl]-methanone was prepared following similar method as for the synthesis of [3-chloro-6-(1H-pyrazol-4-yl)-8-trifluoromethyl-imidazo[1,2-a]pyridin-2-yl]-[3-(2-fluoro-phenyl)-pyrrolidin-1-yl]-methanone (Compound 257). ¹H NMR (d₆-DMSO, 300 MHz) δ 2.00-2.31 (m, 2H), 3.44-4.12 (m, 4.5H), 4.27 (dd, 0.5H, J=7.6, 11.4 Hz), 7.02-7.43 (m, 4H), 8.16 (s, 0.5H), 8.19 (s, 0.5H), 8.23 (brs, 1H), 8.53 (brs, 1H), 8.81 (s, 0.5H), 8.82 (s, 0.5H), 13.13 (brs, 1H); MS (ESI) m/z=478.1 (MH⁺).

### Example 159

### (3-Chloro-6-furan-3-yl-8-trifluoromethyl-imidaz [1,2-a]pyridin-2-yl)-(3-phenyl-2,5-dihydro-pyrrol-1-yl)-methanone (Compound 259)

Under standard HATU coupling conditions, 3-chloro-6-furan-3-yl-8-trifluoromethyl-imidazo[1,2-a]pyridine-2-carboxylic acid, and 3-phenyl-2,5-dihydro-1H-pyrrole (prepared from dehydration of 3-phenyl-pyrrolidin-3-ol) gave (3-chloro-6-furan-3-yl-8-trifluoromethyl-imidazo[1,2-a]pyridin-2-yl)-(3-phenyl-2,5-dihydro-pyrrol-1-yl)-methanone. ¹H NMR (d₆-DMSO, 300 MHz) δ 4.53 (m, 1H), 4.73 (m, 1H), 4.85 (m, 1H), 5.04 (m, 1H), 6.49 (m, 1H), 7.26-7.56 (m, 6H), 7.83 (q, 1H, J=1.4 Hz), 8.21 (dd, 1H, J=1.4, 2.3 Hz), 8.55 (s, 1H), 8.82 (s, 1H); MS (ESI) m/z=458.1 (MH⁺).

### Example 160

### (3-Chloro-6-furan-3-yl-8-trifluoromethyl-imidazo[1,2-a]pyridin-2-yl)-(3-phenyl-pyrrolidin-1-yl)-methanone (Compound 260)

Under standard HATU coupling conditions, 3-chloro-6-furan-3-yl-8-trifluoromethyl-imidazo[1,2-a]pyridine-2-carboxylic acid, and 3-phenyl-pyrrolidine gave (3-chloro-6-furan-3-yl-8-trifluoromethyl-imidazo[1,2-a]pyridin-2-yl)-(3-phenyl-pyrrolidin-1-yl)-methanone. ¹H NMR (d₆-DMSO, 300 MHz) δ 1.98-2.36 (m, 2H), 3.40-4.12 (m, 4.5H), 4.26 (dd, 0.5H, J=7, 10.8 Hz), 7.20-7.36 (m, 6H), 7.82 (t, 0.5H, J=1.8 Hz), 7.83 (t, 0.5H, J=1.8 Hz), 8.16 (brs, 0.5H), 8.19 (brs, 0.5H), 8.53 (brs, 0.5H), 8.55 (brs, 0.5H), 8.79 (brs, 0.5H), 8.81 (brs, 0.5H); MS (ESI) m/z=460.1 (MH⁺).

### Example 161

### (3-Chloro-6-furan-3-yl-8-trifluoromethyl-imidazo[1,2-a]pyridin-2-yl)-(3-(R)-phenyl-pyrrolidin-1-yl)-methanone (Compound 261)

Under standard HATU coupling conditions, 3-chloro-6-furan-3-yl-8-trifluoromethyl-imidazo[1,2-a]pyridine-2-carboxylic acid, and 3R-phenyl-pyrrolidine gave (3-chloro-6-furan-3-yl-8-trifluoromethyl-imidazo[1,2-a]pyridin-2-yl)-((R)-3-phenyl-pyrrolidin-1-yl)-methanone. ¹H NMR (d₆-DMSO, 300 MHz) δ 1.98-2.36 (m, 2H), 3.40-4.12 (m, 4.5H), 4.26 (dd, 0.5H, J=7, 10.8 Hz), 7.20-7.36 (m, 6H), 7.82 (t, 0.5H, J=1.8 Hz), 7.83 (t, 0.5H), J=1.8 Hz), 8.16 (brs, 0.5H), 8.19 (brs, 0.5H), 8.53 (brs, 0.5H), 8.55 (brs, 0.5H), 8.79 (brs, 0.5H), 8.81 (brs, 0.5H); MS (ESI) m/z=460.1 (MH⁺).

### Example 162

### (3-Chloro-6-furan-3-yl-8-trifluoromethyl-imidazo[1,2-a]pyridin-2-yl)-(3-(S)-phenyl-pyrrolidin-1-yl)-methanone (Compound 262)

Under standard HATU coupling conditions, 3-chloro-6-furan-3-yl-8-trifluoromethyl-imidazo[1,2-a]pyridine-2-carboxylic acid, and 3S-phenyl-pyrrolidine gave (3-chloro-6-furan-3-yl-8-trifluoromethyl-imidazo[1,2-a]pyridin-2-yl)-((S)-3-phenyl-pyrrolidin-1-yl)-methanone. ¹H NMR (d₆-DMSO, 300 MHz) δ 1.98-2.36 (m, 2H), 3.40-4.12 (m, 4.5H), 4.26 (dd, 0.5H, J=7, 10.8 Hz), 7.20-7.36 (m, 6H), 7.82 (t, 0.5H, J=1.8 Hz), 7.83 (t, 0.5H), J=1.8 Hz), 8.16 (brs, 0.5H), 8.19 (brs, 0.5H), 8.53 (brs, 0.5H), 8.55 (brs, 0.5H), 8.79 (brs, 0.5H), 8.81 (brs, 0.5H); MS (ESI) m/z=460.1 (MH⁺).

### Example 163

### 3-Chloro-8-furan-3-yl-imidazo[1,2-a]pyridine-2-carboxylic Acid (thiophen-2-ylmethyl)-amide (Compound 263)

A stirred solution of 8-bromo-3-chloro-6-phenyl-imidazo[1,2-a]pyridine-2-carboxylic acid (thiophen-2-ylmethyl)-amide (75 mg, 0.168 mmol), 3-furanboronic acid (28.2 mg, 0.252 mmol), Pd(PPh₃)₄ (19.4 mg, 0.017 mmol) was heated in aqueous K₃PO₄ (560 μL, 1.68 mmol) and 1,4-dioxane (2 mL) at 80° C. for 12 h. The mixture was diluted with EtOAc (20 mL), washed with saturated aqueous NaHCO₃ (10 mL), brine (10 mL), dried (Na₂SO₄), filtered and concentrated. The product was precipitated from ACN, filtered, washed with ether, and dried under high vacuum to afford 3-chloro-8-furan-3-yl-imidazo[1,2-a]pyridine-2-carboxylic acid (thiophen-2-ylmethyl)-amide (compound 263) (35 mg, 48%) as a brown solid. ¹HNMR (d₆-DMSO, 300 MHz) δ 4.65 (d, 2H, J=6.00 Hz), 6.96 (m, 1H), 7.03 (d, 1H, J=1.80 Hz), 7.38 (dd, 1H, J=3.50, 4.70 Hz), 7.55 (m, 4H), 7.85 (m, 3H), 8.10 (s, 1H), 8.44 (s, 1H), 9.33 (s, 1H), 9.47 (t, 1H, J=7.50 Hz); MS (ESI) m/z=434 (MH⁺).

### Example 164

### 3-Chloro-8-(1-methyl-1H-pyrazol-4-yl)-6-phenyl-imidazo[1,2-a]pyridine-2-carboxylic Acid(thiophen-2-ylmethyl)-amide (Compound 264)

3-Chloro-8-(1-methyl-1H-pyrazol-4-yl)-6-phenyl-imidazo[1,2-a]pyridine-2-carboxylic acid (thiophen-2-ylmethyl)-amide (compound 264) was prepared using a similar procedure as for the preparation of 3-chloro-8-furan-3-yl-imidazo[1,2-a]pyridine-2-carboxylic acid (thiophen-2-ylmethyl)-amide (compound 263). MS (ESI) m/z=448.1 (MH⁺).

### Example 165

### 3-Chloro-6-phenyl-8-pyridin-3-yl-imidazo[1,2-a]pyridine-2-carboxylic Acid (thiophen-2-ylmethyl)-amide (Compound 265)

3-Chloro-6-phenyl-8-pyridin-3-yl-imidazo[1,2-a]pyridine-2-carboxylic acid (thiophen-2-ylmethyl)-amide (compound 265) was prepared using a similar procedure as for the preparation of 3-chloro-8-furan-3-yl-imidazo[1,2-a]pyridine-2-carboxylic acid (thiophen-2-ylmethyl)-amide (compound 263). MS (ESI) m/z=446.1 (MH⁺).

### Example 166

### (E/Z)-3-{3-Chloro-6-phenyl-2-[(thiophen-2-ylmethyl)-2-carbamoyl]-imidazo[1,2-a]pyridine-8-yl}-acrylic Acid Methyl Ester (Compound 266)

### Step 1: (E/Z)-3-{3-Chloro-6-phenyl-2-[(thiophen-2-ylmethyl)-2-carbamoyl]-imidazo[1,2-a]pyridine-8-yl}-acrylic Acid Tert-butyl Ester

A stirred solution of 8-bromo-3-chloro-6-phenyl-imidazo[1,2-a]pyridine-2-carboxylic acid (thiophen-2-ylmethyl)-amide (500 mg, 1.12 mmol), tert-butyl acrylate (492 μL, 3.36 mmol), NaOAc (27.5 mg, 3.36 mmol), DIPEA (585 μL, 3.36 mmol), Pd(OAc)₂ (25 mg, 0.112 mmol), and P-(o-tolyl)₃ (34 mg, 0.112 mmol) in DMF (10 mL) was heated at 130° C. under argon for 12 h. The mixture was taken up in water (30 mL) and extracted with EtOAc (3×40 mL), washed with brine (30 mL), dried (Na₂SO₄), filtered and concentrated. Flash chromatography [n-hex/EtOAc (2:1 v/v)] of the crude product gave (E/Z)-3-{3-chloro-6-phenyl-2-[(thiophen-2-ylmethyl)-2-carbamoyl]-imidazo[1,2-a]pyridine-8-yl}-acrylic acid tert-butyl ester (376 mg, 68%) as a brown solid. MS (ESI) m/z=495.1 (MH⁺).

### Step 2: (E/Z)-3-{3-Chloro-6-phenyl-2-[(thiophen-2-ylmethyl)-2-carbamoyl]-imidazo[1,2-a]pyridine-8-yl}-acrylic Acid Methyl Ester (compound 266)

A stirred solution of (E/Z)-3-{3-chloro-6-phenyl-2-[(thiophen-2-ylmethyl)-2-carbamoyl]-imidazo[1,2-a]pyridine-8-yl}-acrylic acid tert-butyl ester (100 mg, 0.202 mmol) in MeOH (2 mL) and 4M HCl in dioxanes (2 mL) was heated 80° C. for 1 hour. Upon cooling, the mixture was co-evaporated with toluene (5 mL) to afford (E/Z)-3-{3-chloro-6-phenyl-2-[(thiophen-2-ylmethyl)-2-carbamoyl]-imidazo[1,2-a]pyridine-8-yl}-acrylic acid methyl ester (66.8 mg, 73%) as a pale yellow solid. MS (ESI) m/z=452.0 (MH⁺).

### Example 167

### (E/Z)-3-{3-Chloro-6-phenyl-2-[(thiophen-2-ylmethyl)-2-carbamoyl]-imidazo[1,2-a]pyridine-8-yl}-acrylic Acid (Compound 267)

A solution of (E/Z)-3-{3-chloro-6-phenyl-2-[(thiophen-2-ylmethyl)-2-carbamoyl]-imidazo[1,2-a]pyridine-8-yl}-acrylic acid tert-butyl ester (110 mg, 0.223 mmol) in TFA (2 mL) and DCM (2 mL) was stirred at 70° C. for 1 hour. Upon cooling, the mixture was co-evaporated with toluene (2×5 mL) to afford (E/Z)-3-{3-chloro-6-phenyl-2-[(thiophen-2-ylmethyl)-2-carbamoyl]-imidazo[1,2-a]pyridine-8-yl}-acrylic acid (compound 267) (93 mg, 95.4%) as a yellow solid. MS (ESI) m/z=438.0 (MH⁺).

### Example 168

### 3-Chloro-8-((E/Z)-2-diethylcarbamoyl-vinyl)-6-phenyl-imidazo[1,2-a]pyridine-2-carboxylic Acid (thiophen-2-ylmethyl)-amide (Compound 268)

A solution of (E/Z)-3-{3-chloro-6-phenyl-2-[(thiophen-2-ylmethyl)-2-carbamoyl]-imidazo[1,2-a]pyridine-8-yl}-acrylic acid (compound 267) (100 mg, 0.228 mmol), diethylamine (60 μL, 0.571 mmol), HATU (130 mg, 0.343 mmol), DIPEA (120 μL, 0.685 mmol) in DMF (1 mL) was stirred at 50° C. for 3 hours. The mixture was diluted with saturated aqueous NaHCO₃ (3 mL) and water (3 mL) followed by extraction with EtOAc (2×10 mL). The organic layer was washed with brine (4 mL), dried (MgSO₄), filtered, and concentrated. The product was purified by preparative HPLC (30-100% gradient ACN/water with 0.1% TFA), and converted to the HCl salt to afford 3-chloro-8-((E/Z)-2-diethylcarbamoyl-vinyl)-6-phenyl-imidazo[1,2-a]pyridine-2-carboxylic acid (thiophen-2-ylmethyl)-amide (compound 268) (66 mg, 59%) as a dark brown solid. ¹H NMR (d₆-DMSO, 300 MHz) δ 1.05 (m, 6H), 3.35 (m, 2H), 3.52 (m, 2H), 4.59 (d, 2H, J=7.80 Hz), 6.90 (m, 1H), 6.97 (dd, 1H, J=1.20, 3.60 Hz), 7.46 (m, 4H), 7.78 (m, 3H), 8.07 (d, 1H, J=15.30 Hz), 8.14 (d, 1H, J=1.50 Hz), 8.47 (d, 1H, J=1.80 Hz), 8.89 (t, 1H, J=6.60 Hz); MS (ESI) m/z=493.1 (MH⁺).

### Example 169

### 1-(3-Chloro-6-furan-3-yl-8-trifluoromethyl-imidazo[1,2-a]pyridine-2-carbonyl)-piperidine-4-carboxylic Acid Ethyl Ester (Compound 269)

Ethyl-4-piperidine carboxylate (93 μL, 0.605 mmol) was added to a stirred solution of 3-chloro-6-furan-3-yl-8-trifluoromethyl-imidazo[1,2-a]pyridine-2-carboxylic acid (100 mg, 0.302 mmol), HATU (173 mg, 0.454 mmol) and DIPEA (158 μL, 0.907 mmol) in DMF (2 mL). The mixture was stirred at 50° C. for 1.5 hours. Saturated aqueous NaHCO₃ (1 mL) was added to the mixture followed by extraction with EtOAc (2×4 mL). The combined organic layer was washed with brine (2 mL), dried (MgSO₄), filtered, and concentrated in vacuo. The product was purified using preparative TLC [n-hex/EtOAc (2:1 v/v)] to afford 1-(3-chloro-6-furan-3-yl-8-trifluoromethyl-imidazo[1,2-a]pyridine-2-carbonyl)-piperidine-4-carboxylic acid ethyl ester (compound 269) (125 mg, 88%) as white solid. MS (ESI) m/z=470.1 (MH⁺).

### Example 170

### 1-(3-Chloro-6-furan-3-yl-8-trifluoromethyl-imidazo[1,2-a]pyridine-2-carbonyl)-piperidine-4-carboxylic Acid (Compound 270)

A mixture of 1-(3-chloro-6-furan-3-yl-8-trifluoromethyl-imidazo[1,2-a]pyridine-2-carbonyl)-piperidine-4-carboxylic acid ethyl ester (300 mg, 0.639 mmol) and 3M LiOH (1.28 mL, 3.830 mmol) in THF (5 mL) was stirred at room temperature for 12 hours. The precipitate was filtered and the cake was washed with THF (2×5 mL). The filtrate was acidified with 10% aqueous HCl, then extracted with EtOAc (2×20 mL). The organic layer was washed with brine, dried (MgSO₄), filtered, and concentrated. The product was purified using preparative TLC [MeOH/CH₂Cl₂ (5:95 v/v)] to afford 1-(3-chloro-6-furan-3-yl-8-trifluoromethyl-imidazo[1,2-a]pyridine-2-carbonyl)-piperidine-4-carboxylic acid (compound 270) (200 mg, 71%) as pale yellow solid. MS (ESI) m/z=442.1 (MH⁺).

### Example 171

### 1-(3-Chloro-6-furan-3-yl-8-trifluoromethyl-imidazo[1,2-a]pyridine-2-carbonyl)-piperidine-4-carboxylic Acid Phenylamide (Compound 271)

Aniline (31 μL, 0.340 mmol) was added to a stirring solution of 1-(3-chloro-6-furan-3-yl-8-trifluoromethyl-imidazo[1,2-a]pyridine-2-carbonyl)-piperidine-4-carboxylic acid (75 mg, 0.170 mmol), HATU (97 mg, 0.255 mmol) and DIPEA (89 μL, 0.509 mmol) in DMF (1 mL). The mixture was stirred at 50° C. for 1.5 h. Saturated aqueous NaHCO₃ (1 mL) was added to the mixture followed by extraction with EtOAc (2×4 mL). The combined organic layer was washed with brine (2 mL), dried (MgSO₄), filtered, and concentrated. The product was purified using preparative TLC [MeOH/CH₂Cl₂ (5:95 v/v)] to afford 1-(3-chloro-6-furan-3-yl-8-trifluoromethyl-imidazo[1,2-a]pyridine-2-carbonyl)-piperidine-4-carboxylic acid phenylamide (compound 271) (26.3 mg, 30%) as a white solid. ¹H NMR (d₆-DMSO, 300 MHz) δ 1.64 (m, 2H), 1.79 (m, 1H), 1.95 (m, 1H), 2.68 (m, 1H), 2.94 (m, 1H), 3.18 (m, 1H), 4.14 (d, 1H, J=8.70 Hz), 4.57 (d, 1H, J=4.50 Hz), 7.03 (m, 1H), 7.32 (m, 4H), 7.58 (dd, 1H, J=1.20, 9.00 Hz), 7.83 (m, 1H), 8.18 (m, 1H), 8.55 (s, 1H), 8.81 (s, 1H), 9.97 (s, 1H); MS (ESI) m/z=517.1 (MH⁺).

### Example 172

### 1-(3-Chloro-6-furan-3-yl-8-trifluoromethyl-imidazo[1,2-a]pyridine-2-carbonyl)-piperidine-4-carboxylic Acid Benzylamide (Compound 272)

1-(3-Chloro-6-furan-3-yl-8-trifluoromethyl-imidazo[1,2-a]pyridine-2-carbonyl)-piperidine-4-carboxylic acid benzylamide (compound 272) was prepared in a similar method as 1-(3-chloro-6-furan-3-yl-8-trifluoromethyl-imidazo[1,2-a]pyridine-2-carbonyl)-piperidine-4-carboxylic acid phenylamide (compound 271). MS (ESI) m/z=531.2 (MH⁺).

### Example 173

### 1-(3-Chloro-6-furan-3-yl-8-trifluoromethyl-imidazo[1,2-a]pyridine-2-carbonyl)-piperidine-4-carboxylic Acid Ethylamide (Compound 273)

1-(3-Chloro-6-furan-3-yl-8-trifluoromethyl-imidazo[1,2-a]pyridine-2-carbonyl)-piperidine-4-carboxylic acid ethylamide (compound 273) was prepared in a similar method as 1-(3-chloro-6-furan-3-yl-8-trifluoromethyl-imidazo[1,2-a]pyridine-2-carbonyl)-piperidine-4-carboxylic acid phenylamide (compound 271). MS (ESI) m/z=469.1 (MH⁺).

### Example 174

### 1-(3-Chloro-6-furan-3-yl-8-trifluoromethyl-imidazo[1,2-a]pyridine-2-carbonyl)-piperidine-4-carboxylic Acid Diethylamide (Compound 274)

1-(3-Chloro-6-furan-3-yl-8-trifluoromethyl-imidazo[1,2-a]pyridine-2-carbonyl)-piperidine-4-carboxylic acid diethylamide (compound 274) was prepared in a similar method as 1-(3-chloro-6-furan-3-yl-8-trifluoromethyl-imidazo[1,2-a]pyridine-2-carbonyl)-piperidine-4-carboxylic acid phenylamide (compound 271). MS (ESI) m/z=497.2 (MH⁺).

### Example 175

### (3-Chloro-6-furan-3-yl-8-trifluoromethyl-imidaz[1,2-a]pyridine-2-yl)-[4-(2-fluoro-phenyl)-piperidin-1-yl]-methanone (Compound 275)

4-(2-Fluorophenyl)piperidine hydrochloride (130 mg, 0.605 mmol) was added to a stirring solution of 3-chloro-6-furan-3-yl-8-trifluoromethyl-imidazo[1,2-a]pyridine-2-carboxylic acid (100 mg, 0.302 mmol), HATU (138 mg, 0.363 mmol) and DIPEA (158 μL, 0.907 mmol) in DMF (2 mL). The mixture was stirred at 50° C. for 1.5 h. Saturated aqueous NaHCO₃ (1 mL) was added followed by extraction with EtOAc (2×4 mL). The combined organic layer was washed with brine (2 mL), dried (MgSO₄), filtered, and concentrated. The product was purified using preparative TLC [MeOH/CH₂Cl₂ (5:95 v/v)] to afford (3-chloro-6-furan-3-yl-8-trifluoromethyl-imidazo[1,2-a]pyridine-2-yl)-[4-(2-fluoro-phenyl)-piperidin-1-yl]-methanone (compound 275) (45 mg, 30%) as a white solid. ¹H NMR (d₆-DMSO, 300 MHz) δ 1.69 (m, 3H), 1.90 (d, 1H, J=12.6 Hz), 2.95 (m, 1H), 3.20 (m, 2H), 4.19 (d, 1H, J=12.9 Hz), 4.68 (d, 1H, J=13.2 Hz), 7.14 (m, 2H), 7.24 (m, 1H), 7.31 (m, 2H), 7.82 (t, 1H, J=1.5 Hz), 8.18 (s, 1H), 8.54 (s, 1H), 8.80 (s, 1H); MS (ESI) m/z=492.1 (MH⁺).

### Example 176

### (3-Chloro-6-furan-3-yl-8-trifluoromethyl-imidaz[1,2-a]pyridine-2-yl)-[4-(3-fluoro-phenyl)-piperidin-1-yl]-methanone (Compound 276)

(3-Chloro-6-furan-3-yl-8-trifluoromethyl-imidazo[1,2-a]pyridine-2-yl)-[4-(3-fluoro-phenyl)-piperidin-1-yl]-methanone (compound 276) was prepared using a similar method as (3-chloro-6-furan-3-yl-8-trifluoromethyl-imidazo[1,2-a]pyridine-2-yl)-[4-(2-fluoro-phenyl)-piperidin-1-yl]-methanone (compound 275). ¹NMR (d₆-DMSO, 300 MHz) δ 1.63 (m, 2H), 1.80 (d, 1H, J=12.3 Hz), 1.93 (d, 1H, J=11.4 Hz), 2.90 (m, 2H), 3.22 (m, 1H), 4.19 (d, 1H, J=12.9 Hz), 4.67 (d, 1H, J=13.2 Hz), 7.01 (m, 1H), 7.05 (s, 1H), 7.12 (d, 1H, J=1.5 Hz), 7.31 (m, 2H), 7.82 (t, 1H, J=1.8 Hz), 8.18 (d, 1H, J=1.5 Hz), 8.54 (d, 1H, J=1.2 Hz), 8.80 (s, 1H); MS (ESI) m/z=492.1 (MH⁺).

### Example 177

### (3-Chloro-6-furan-3-yl-8-trifluoromethyl-imidazo[1,2-a]pyridine-2-yl)-[4-(4-fluoro-phenyl)-piperidin-1-yl]-methanone (Compound 277)

(3-Chloro-6-furan-3-yl-8-trifluoromethyl-imidazo[1,2-a]pyridine-2-yl)-[4-(4-fluoro-phenyl)-piperidin-1-yl]-methanone (compound 277) was prepared using a similar method as (3-chloro-6-furan-3-yl-8-trifluoromethyl-imidazo[1,2-a]pyridine-2-yl)-[4-(2-fluoro-phenyl)-piperidin-1-yl]-methanone (compound 275). ¹H NMR (d₆-DMSO, 300 MHz) δ 1.60 (m, 2H), 1.76 (d, 1H, J=10.60 Hz), 1.90 (d, 1H, J=10.50 Hz), 2.87 (m, 2H), 3.22 (m, 1H), 4.18 (d, 1H, J=13.20 Hz), 4.67 (d, 1H, J=13.20 Hz), 7.11 (m, 2H), 7.30 (m, 3H), 7.82 (t, 1H, J=1.80 Hz), 8.17 (s, 1H), 8.53 (d, 1H, J=1.20 Hz), 8.80 (s, 1H); MS (ESI) m/z=492.1 (MH⁺).

### Example 178

### 3-Chloro-6-(3-dimethylaminomethyl-phenyl)-8-trifluoromethyl-imidazo[1,2-a]pyridine-2-carboxylic Acid (thiophen-2-ylmethyl)-amide (Compound 278)

A stirred mixture of 6-bromo-3-chloro-8-trifluoromethyl-imidazo[1,2-a]pyridine-2-carboxylic acid (thiophen-2-ylmethyl)-amide 12 (50 mg, 0.114 mmol), 3-(N,N-dimethylaminomethyl)phenylboronic acid pinacol ester (68 mg, 0.228 mmol), and Pd(PPh₃)₄ (13 mg, 0.011 mmol) in aqueous K₃PO₄ (380 μL, 1.140 mmol) and 1,4-dioxanes (1 mL) was heated at 80° C. for 12 hours. The mixture was diluted with EtOAc (20 mL), washed with saturated aqueous NaHCO₃ (10 mL), then brine (10 mL). The extracts were dried (Na₂SO₄), filtered, and concentrated. The product was purified using preparative TLC [MeOH/CH₂Cl₂ (13:87 v/v)] to afford 3-chloro-6-(3-dimethylaminomethyl-phenyl)-8-trifluoromethyl-imidazo[1,2-a]pyridine-2-carboxylic acid (thiophen-2-ylmethyl)-amide (compound 278) (25 mg, 45%) as an off-white solid. ¹H NMR (d₆-DMSO, 300 MHz) δ 2.21 (s, 6H), 3.53 (s, 2H), 4.65 (d, 2H, J=5.70 Hz), 6.95 (dd, 1H, J=3.30, 5.10 Hz), 7.03 (m, 1H), 7.44 (m, 4H), 7.76 (m, 1H), 8.17 (s, 1H), 8.77 (d, 1H, J=3.00 Hz), 8.88 (t, 1H, J=6.00 Hz); MS (ESI) m/z=493.1 (MH⁺).

### Example 179

### 3-Chloro-6-(1H-pyrrol-3-yl)-8-trifluoromethyl-imidazo[1,2-a]pyridine-2-carboxylic Acid(thiophen-2-ylmethyl)-amide (Compound 279)

3-Chloro-8-trifluoromethyl-6-(1-triisopropylsilanyl-1H-pyrrol-3-yl)-imidazo[1,2a]pyridine-2-carboxylic acid (thiophen-2-ylmethyl)-amide was prepared via Suzuki coupling of 6-bromo-3-chloro-8-trifluoromethyl-imidazo[1,2-a]pyridine-2-carboxylic acid (thiophen-2-ylmethyl)-amide with 1-(triisopropylsilyl)pyrrole-3-boronic acid. Deprotection was accomplished by stirring a solution of the above (0.28 g, 0.48 mmol) with K₂CO₃ (0.27 g, 2 mmol) in MeOH (10 mL) for 3 hours. The crude reaction mixture was filtered and the filtrate concentrated under reduced pressure. The crude material was diluted with water and EtOAc. The organic layer was separated and washed successively with saturated aqueous NaHCO₃, water, and brine. The extracts were dried (Na₂SO₄), filtered and concentrated. The product was purified by preparative HPLC to afford 3-chloro-6-(1H-pyrrol-3-yl)-8-trifluoromethyl-imidazo[1,2-a]pyridine-2-carboxylic acid (thiophen-2-ylmethyl)-amide (compound 279) (0.016 g, 8%). ¹H NMR (d₆-DMSO, 300 MHz) δ 4.63 (d, 2H, J=6.0 Hz), 6.70 (br s, 1H), 6.88 (m, 1H), 6.96 (m, 1H), 7.03 (m, 1H), 7.37 (d, 1H, J=5.1 Hz), 7.59 (s, 1H), 8.14 (s, 1H), 8.58 (s, 1H), 8.81 (t, 1H, J=6.0 Hz), 11.19 (s, 1H); MS 424.9 (MH⁺).

### Example 180

### 3-Chloro-6-(1-methyl-1H-pyrazol-4-yl)-8-trifluoromethyl-imidazo[1,2-a]pyridine-2-carboxylic Acid (thiophen-2-ylmethyl)-amide (Compound 280)

3-Chloro-6-(1-methyl-1H-pyrazol-4-yl)-8-trifluoromethyl-imidazo[1,2-a]pyridine-2-carboxylic acid (thiophen-2-ylmethyl)-amide (compound 280) was prepared using a similar method as for the preparation of 3-chloro-6-(1H-pyrrol-3-yl)-8-trifluoromethyl-imidazo[1,2-a]pyridine-2-carboxylic acid (thiophen-2-ylmethyl)-amide (compound 279). ¹H NMR (d₆-DMSO, 300 MHz) δ 3.88 (s, 3H), 4.63 (d, 2H, J=6.3 Hz), 6.94 (dd, 1H, J=3.3, 5.1 Hz), 7.02 (d, 1H, J=3.3 Hz), 7.36 (brd, 1H, J=4.8 Hz), 8.14 (s, 1H), 8.16 (s, 1H), 8.47 (s, 1H), 8.77 (s, 1H), 8.81 (t, 1H, J=6.3 Hz); MS (ESI) m/z=440 (MH⁺).

### Example 181

### 2-{3-Chloro-2-[(thiophen-2-ylmethyl)-carbamoyl]-8-trifluoromethyl-imidazo[1,2-a]pyridin-6-yl}-pyrrole-1-carboxylic Acid Tert-butyl Ester (Compound 281)

2-{3-Chloro-2-[(thiophen-2-ylmethyl)-carbamoyl]-8-trifluoromethyl-imidazo[1,2-a]pyridin-6-yl}-pyrrole-1-carboxylic acid tert-butyl ester (compound 281) was prepared using a similar method as for the preparation of 3-chloro-6-(1H-pyrrol-3-yl)-8-trifluoromethyl-imidazo[1,2-a]pyridine-2-carboxylic acid (thiophen-2-ylmethyl)-amide (compound 279). ¹H NMR (d₆-DMSO, 300 MHz) δ 1.3 (s, 9H), 4.64 (d, 2H, J=6.0 Hz), 6.35 (t, 1H, J=3.3 Hz), 6.53 (m, 1H), 6.95 (dd, 1H, J=3.6, 5.1 Hz), 7.00 (m, 1H), 7.36 (d, 1H, J=5.1 Hz), 7.48 (m, 1H), 8.09 (s, 1H), 8.62 (s, 1H), 8.80 (t, 1H, J=5.7 Hz); MS (ESI) m/z=525 (MH⁺).

### Example 182

### 3-Chloro-6-cyclohex-1-enyl-8-trifluoromethyl-imidaz[1,2-a]pyridine-2-carboxylic Acid(thiophen-2-ylmethyl)-amide (Compound 282)

3-Chloro-6-cyclohex-1-enyl-8-trifluoromethyl-imidazo[1,2-a]pyridine-2-carboxylic acid (thiophen-2-ylmethyl)-amide (compound 282) was prepared using a similar method as for the preparation of 3-chloro-6-(1H-pyrrol-3-yl)-8-trifluoromethyl-imidazo[1,2-a]pyridine-2-carboxylic acid (thiophen-2-ylmethyl)-amide (compound 279). ¹H NMR (d₆-DMSO, 300 MHz) δ 1.55-1.80 (m, 4H), 2.22 (m, 2H), 2.44 (m, 2H), 4.62 (d, 2H, J=6.6 Hz), 6.48 (t, 1H, J=3.9 Hz), 6.94 (dd, 1H, J=3.6, 5.1 Hz), 7.01 (d, 1H, J=2.7 Hz), 7.36 (dd, 1H, J=1.2, 5.1 Hz), 8.02 (s, 1H), 8.31 (s, 1H), 8.83 (t, 1H, J=6.6 Hz); MS (ESI) m/z=440 (MH⁺).

### Example 183

### 3-Chloro-6-(2H-pyrazol-3-yl)-8-trifluoromethyl-imidazo[1,2-a]pyridine-2-carboxylic Acid(thiophen-2-ylmethyl)-amide (Compound 283)

3-Chloro-6-(2H-pyrazol-3-yl)-8-trifluoromethyl-imidazo[1,2-a]pyridine-2-carboxylic acid (thiophen-2-ylmethyl)-amide (compound 283) was prepared using a similar method as for the preparation of 3-chloro-6-(1H-pyrrol-3-yl)-8-trifluoromethyl-imidazo[1,2-a]pyridine-2-carboxylic acid (thiophen-2-ylmethyl)-amide (compound 279). ¹H NMR (d₆-DMSO, 300 MHz) δ 4.63 (d, 2H, J=6.0 Hz), 6.94 (m, 1H), 7.02 (m, 1H), 7.09 (d, 1H, J=2.1 Hz), 7.36 (d, 1H, J=4.5 Hz), 7.86 (s, 1H), 8.32 (s, 1H), 8.86 (t, 1H, J=6.0 Hz), 8.90 (s, 1H); MS 425.9 (MH⁺), 447.9 (MNa⁺).

### Example 184

### 3-Chloro-6-(5,6-dihydro-4H-pyran-2-yl)-8-trifluoromethyl-imidazo[1,2-a]pyridine-2-carboxylic Acid (thiophen-2-ylmethyl)-amide (Compound 284)

3-Chloro-6-(5,6-dihydro-4H-pyran-2-yl)-8-trifluoromethyl-imidazo[1,2-a]pyridine-2-carboxylic acid (thiophen-2-ylmethyl)-amide (compound 284) was prepared using a similar method as for the preparation of 3-chloro-6-(1H-pyrrol-3-yl)-8-trifluoromethyl-imidazo[1,2-a]pyridine-2-carboxylic acid (thiophen-2-ylmethyl)-amide (compound 279). ¹H NMR (d₆-DMSO, 300 MHz) δ 1.94 (m, 2H), 2.28 (m, 2H), 4.26 (t, 2H, J=4.5 Hz), 4.69 (d, 2H, J=6 Hz), 5.95 (t, 1H, J=4.2 Hz), 7.00 (dd, 1H, J=3.6, 5.1 Hz), 7.08 (dd, 1H, J=1.2, 3.3 Hz), 7.43 (dd, 1H, J=1.2, 5.1 Hz), 8.12 (s, 1H), 8.46 (s, 1H), 8.91 (t, 1H, J=6 Hz); MS (ESI) m/z=442 (MH⁺).

### Example 185

### 6-(1-Benzyl-1H-pyrazol-4-yl)-3-chloro-8-trifluoromethyl-imidazo[1,2-a]pyridine-2-carboxylic Acid (thiophen-2-ylmethyl)-amide (Compound 285)

6-(1-Benzyl-1H-pyrazol-4-yl)-3-chloro-8-trifluoromethyl-imidazo[1,2-a]pyridine-2-carboxylic acid (thiophen-2-ylmethyl)-amide (compound 285) was prepared using a similar method as for the preparation of 3-chloro-6-(1H-pyrrol-3-yl)-8-trifluoromethyl-imidazo[1,2-a]pyridine-2-carboxylic acid (thiophen-2-ylmethyl)-amide (compound 279). ¹H NMR (d₆-DMSO, 300 MHz) δ 4.63 (d, 2H, J=6.3 Hz), 5.36 (s, 2H), 6.94 (dd, 1H, J=3.6, 5.4 Hz), 7.02 (m, 1H), 7.33 (m, 6H), 8.18 (s, 1H), 8.24 (s, 1H), 8.64 (s, 2H), 8.83 (m, 1H); MS (ESI) m/z=516 (MH⁺).

### Example 186

### 3-Chloro-6-(3-dimethylamino-phenyl)-8-trifluoromethyl-imidazo[1,2-a]pyridine-2-carboxylic Acid (thiophen-2-ylmethyl)-amide (Compound 286)

3-Chloro-6-(3-dimethylamino-phenyl)-8-trifluoromethyl-imidazo[1,2-a]pyridine-2-carboxylic acid (thiophen-2-ylmethyl)-amide (compound 286) was prepared using a similar method as for the preparation of 3-chloro-6-(1H-pyrrol-3-yl)-8-trifluoromethyl-imidazo[1,2-a]pyridine-2-carboxylic acid (thiophen-2-ylmethyl)-amide (compound 279). ¹H NMR (d₆-DMSO, 300 MHz) δ 3.12 (s, 6H), 4.71 (d, 2H, J=6 Hz), 7.01 (dd, 1H, J=3.6, 5.4 Hz), 7.10 (dd, 1H, J=0.6, 3.6 Hz), 7.16 (brs, 1H), 7.44 (dd, 1H, J=1.5, 5.4 Hz), 7.32-7.54 (m, 3H), 8.24 (s, 1H), 8.31 (s, 1H), 8.96 (t, 1H, J=6 Hz); MS (ESI) m/z=479.1 (MH⁺).

### Example 187

### 3-Chloro-6-styryl-8-trifluoromethyl-imidazo[1,2-a]pyridine-2-carboxylic Acid (thiophen-2-ylmethyl)-amide (Compound 287)

3-Chloro-6-styryl-8-trifluoromethyl-imidazo[1,2-a]pyridine-2-carboxylic acid (thiophen-2-ylmethyl)-amide (compound 287) was prepared using a similar method as for the preparation of 3-chloro-6-(1H-pyrrol-3-yl)-8-trifluoromethyl-imidazo[1,2-a]pyridine-2-carboxylic acid (thiophen-2-ylmethyl)-amide (compound 279). ¹H NMR (d₆-DMSO, 300 MHz) δ 4.63 (d, 2H, J=6.6 Hz), 6.94 (m, 1H), 7.02 (m, 1H), 7.46-7.30 (m, 4H), 7.52 (s, 1H), 7.55 (s, 1H), 7.61 (d, 2H, J=7.2 Hz), 8.34 (s, 1H), 8.79 (s, 1H), 8.86 (t, 1H, J=6.6 Hz); MS (ESI) m/z=462.0 (MH⁺), 484.0 (MNa⁺).

### Example 188

### 3-Chloro-6-isoxazol-4-yl-8-trifluoromethyl-imidazo[1,2-a]pyridine-2-carboxylic Acid(thiophen-2-ylmethyl)-amide (Compound 288)

3-Chloro-6-isoxazol-4-yl-8-trifluoromethyl-imidazo[1,2-a]pyridine-2-carboxylic acid (thiophen-2-ylmethyl)-amide (compound 288) was prepared using a similar method as for the preparation of 3-chloro-6-(1H-pyrrol-3-yl)-8-trifluoromethyl-imidazo[1,2-a]pyridine-2-carboxylic acid (thiophen-2-ylmethyl)-amide (compound 279). ¹H NMR (d₆-DMSO, 300 MHz) δ 4.63 (d, 2H, J=6.0 Hz), 6.94 (m, 1H), 7.03 (m, 1H), 7.36 (m, 1H), 8.29 (s, 1H), 8.88 (t, 1H, J=5.7 Hz), 9.04 (s, 1H), 9.46 (s, 1H), 9.73 (s, 1H); MS (ESI) m/z=427 (MH⁺).

### Example 189

### 3-Chloro-6-(2,4-dimethyl-thiazol-5-yl)-8-trifluoromethyl-imidazo[1,2-a]pyridine-2-carboxylic Acid (thiophen-2-ylmethyl)-amide (Compound 289)

3-Chloro-6-(2,4-dimethyl-thiazol-5-yl)-8-trifluoromethyl-imidazo[1,2-a]pyridine-2-carboxylic acid (thiophen-2-ylmethyl)-amide (compound 289) was prepared using a similar method as for the preparation of 3-chloro-6-(1H-pyrrol-3-yl)-8-trifluoromethyl-imidazo[1,2-a]pyridine-2-carboxylic acid (thiophen-2-ylmethyl)-amide (compound 279). MS (ESI) m/z=471.0.0 (MH⁺).

### Example 190

### 3-Chloro-6-(1H-pyrazol-4-yl)-8-trifluoromethyl-imidazo[1,2-a]pyridine-2-carboxylic Acid(thiophen-2-ylmethyl)-amide (Compound 290)

3-Chloro-6-(1H-pyrazol-4-yl)-8-trifluoromethyl-imidazo[1,2-a]pyridine-2-carboxylic acid (thiophen-2-ylmethyl)-amide (compound 290) was prepared using a similar method as for the preparation of 3-chloro-6-(1H-pyrrol-3-yl)-8-trifluoromethyl-imidazo[1,2-a]pyridine-2-carboxylic acid (thiophen-2-ylmethyl)-amide (compound 279). ¹H NMR (d₆-DMSO): δ 4.63 (d, 2H, J=6 Hz), 6.94 (m, 1H), 7.02 (brs, 1H), 7.35 (d, 1H, J=4.8 Hz), 8.20 (s, 1H), 8.39 (s, 2H), 8.80 (m, 2H); MS (ESI) m/z=426.0 (MH⁺).

### Example 191

### 3-{3-Chloro-2-[(thiophen-2-ylmethyl)-carbamoyl]-8-trifluoromethyl-imidazo[1,2-a]pyridin-6-yl}-benzoic Acid Methyl Ester (Compound 291)

3-{3-Chloro-2-[(thiophen-2-ylmethyl)-carbamoyl]-8-trifluoromethyl-imidazo[1,2-a]pyridin-6-yl}-benzoic acid methyl ester (compound 291) was prepared using a similar method as for the preparation of 3-chloro-6-(1H-pyrrol-3-yl)-8-trifluoromethyl-imidazo[1,2-a]pyridine-2-carboxylic acid (thiophen-2-ylmethyl)-amide (compound 279). MS (ESI) m/z=494.0 (MH⁺).

### Example 192

### 3-Chloro-6-[1-(2-morpholin-4-yl-ethyl)-1H-pyrazol-4-yl]-8-trifluoromethyl-imidazo[1,2-a]pyridine-2-carboxylic acid (thiophen-2-ylmethyl)-amide (Compound 292)

3-Chloro-6-[1-(2-morpholin-4-yl-ethyl)-1H-pyrazol-4-yl]-8-trifluoromethyl-imidazo[1,2-a]pyridine-2-carboxylic acid (thiophen-2-ylmethyl)-amide (compound 292) was prepared using a similar method as for the preparation of 3-chloro-6-(1H-pyrrol-3-yl)-8-trifluoromethyl-imidazo[1,2-a]pyridine-2-carboxylic acid (thiophen-2-ylmethyl)-amide (compound 279). MS (ESI) m/z=539.1 (MH⁺).

### Example 193

### 3-Chloro-6-(1H-pyrrol-2-yl)-8-trifluoromethyl-imidazo[1,2-a]pyridine-2-carboxylic Acid(thiophen-2-ylmethyl)-amide (Compound 293)

A mixture of 2-{3-chloro-2-[(thiophen-2-ylmethyl)-carbamoyl]-8-trifluoromethyl-imidazo[1,2-a]pyridin-6-yl}-pyrrole-1-carboxylic acid tert-butyl ester (0.034 μm, 0.06 mmol) and HCl (4M solution in 1,4-dioxane, 2 mL) was stirred for 72 hours. Concentration of the solvent followed by drying under high vacuum gave 3-chloro-6-(1H-pyrrol-2-yl)-8-trifluoromethyl-imidazo[1,2-a]pyridine-2-carboxylic acid (thiophen-2-ylmethyl)-amide (compound 293) (0.01 g, 39%). ¹H NMR (d₆-DMSO, 300 MHz) δ 4.63 (d, 2H, J=6.30 Hz), 6.17 (s, 1H), 6.95 (m, 1H), 7.00 (m, 1H), 7.36 (d, 1H, J=5.1 Hz), 8.18 (s, 1H), 8.82 (m, 2H), 11.71 (s, 1H); MS (ESI) m/z=425 (MH⁺).

### Example 194

### 3-Chloro-6-phenylethynyl-8-trifluoromethyl-imidazo[1,2-a]pyridine-2-carboxylic Acid(thiophen-2-ylmethyl)-amide (Compound 294)

A mixture of 6-bromo-3-chloro-8-trifluoromethyl-imidazo[1,2-a]pyridine-2-carboxylic acid (thiophen-2-ylmethyl)-amide (0.132 g, 0.3 mmol), phenyl acetylene (0.066 mL, 0.45 mmol), bis(triphenylphosphine)palladium(II) chloride (0.015 g, 0.021 mmol), copper(I) iodide (0.015 g, 0.078 mmol), triethylamine (0.3 mL, 2.11 mmol) in DMF (1.2 mL) was heated at 100° C. for 3 min under microwave conditions. The crude product was purified via silica gel chromatography to afford 3-chloro-6-phenylethynyl-8-trifluoromethyl-imidazo[1,2-a]pyridine-2-carboxylic acid (thiophen-2-ylmethyl)-amide (compound 294) (0.0126 g, 9%). ¹H NMR (dₛ-DMSO, 300 MHz) δ 4.64 (d, 2H, J=5.7 Hz), 6.90-7.10 (m, 2H), 7.30-7.70 (m, 6H), 8.04 (s, 1H), 8.90 (t, 1H, J=5.7 Hz), 8.97 (s, 1H); MS (ESI) m/z=460 (MH⁺).

### Example 195

### 3-Chloro-6-(4-hydroxy-but-1-ynyl)-8-trifluoromethyl-imidazo[1,2-a]pyridine-2-carboxylic Acid (thiophen-2-ylmethyl)-amide (Compound 295)

3-Chloro-6-(4-hydroxy-but-1-ynyl)-8-trifluoromethyl-imidazo[1,2-a]pyridine-2-carboxylic acid (thiophen-2-ylmethyl)-amide (compound 295) was prepared using Sonogashira protocol similar to the preparation of 3-chloro-6-phenylethynyl-8-trifluoromethyl-imidazo[1,2-a]pyridine-2-carboxylic acid (thiophen-2-ylmethyl)-amide (compound 294). MS (ESI) m/z=428.0 (MH⁺).

### Example 196

### 3-Chloro-6-(3-hydroxy-prop-1-ynyl)-8-trifluoromethyl-imidazo[1,2-a]pyridine-2-carboxylic Acid (thiophen-2-ylmethyl)-amide (Compound 296)

3-Chloro-6-(3-hydroxy-prop-1-ynyl)-8-trifluoromethyl-imidazo[1,2-a]pyridine-2-carboxylic acid (thiophen-2-ylmethyl)-amide (compound 296) was prepared using Sonogashira protocol similar to the preparation of 3-chloro-6-phenylethynyl-8-trifluoromethyl-imidazo[1,2-a]pyridine-2-carboxylic acid (thiophen-2-ylmethyl)-amide (compound 294). MS (ESI) m/z=414.0 (MH⁺), 436.0 (MNa⁺).

### Example 197

### 3-Chloro-6-ethynyl-8-trifluoromethyl-imidazo[1,2-a]pyridine-2-carboxylic Acid (thiophen-2-ylmethyl)-amide (Compound 297)

3-Chloro-8-trifluoromethyl-6-trimethylsilanylethynyl-imidazo[1,2-a]pyridine-2-carboxylic acid (thiophen-2-ylmethyl)-amide [prepared via Sonogashira coupling as in the preparation of 3-chloro-6-phenylethynyl-8-trifluoromethyl-imidazo[1,2-a]pyridine-2-carboxylic acid (thiophen-2-ylmethyl)-amide (compound 294)](0.09 g, 0.2 mmol) was stirred in THF (10 mL) at 0° C. and Et₃N.3HF solution (0.035 mL, 0.3 mmol) was added. The mixture was allowed to warm to room temperature and stirred for 3 hours. The crude reaction mixture was quenched with silica gel, filtered and the crude product obtained from a normal extractive workup was purified by silica gel chromatography to afford the title compound (0.015 g, 19%). ¹H NMR (d₆-DMSO, 300 MHz) δ 4.55 (s, 1H), 4.61 (d, 2H, J=6.6 Hz), 6.94 (m, 1H), 7.01 (m, 1H), 7.35 (dd, 1H, J=0.9, 4.8 Hz), 7.91 (s, 1H), 8.88 (m, 2H). MS (ESI) m/z=384.0 (MH⁺).

### Example 198

### 6-(3-Fluoro-phenyl)-3-iodo-8-trifluoromethyl-imidazo[1,2-a]pyridine-2-carboxylic Acid (Compound 298)

6-Bromo-8-trifluoromethyl-imidazo[1,2-a]pyridine-2-carboxylic acid ethyl ester was subjected to Suzuki coupling conditions with 3-fluorophenylboronic acid to afford 6-(3-fluoro-phenyl)-8-trifluoromethyl-imidazo[1,2-a]pyridine-2-carboxylic acid ethyl ester. This compound was saponified with aqueous NaOH to afford 6-(3-fluoro-phenyl)-8-trifluoromethyl-imidazo[1,2-a]pyridine-2-carboxylic acid. This acid (2.14 gm, 6.6 mmol) was iodinated with N-iodosuccinimide (1.9 g, 8.4 mmol) in DMF (30 mL) for 18 h. The mixture was poured into water to give a precipitate which was filtered, and dried under high vacuum to afford 6-(3-Fluoro-phenyl)-3-iodo-8-trifluoromethyl-imidazo[1,2-a]pyridine-2-carboxylic acid (compound 298) in quantitative yield. ¹H NMR (d₆-DMSO, 300 MHz) δ 7.31 (dt, 1H, J=2.7, 8.1 Hz), 7.58 (m, 1H), 7.68 (d, 1H, J=8.1 Hz), 7.77 (d, 1H, J=10.2 Hz), 8.19 (s, 1H), 8.73 (s, 1H); MS (ESI) m/z=450.9 (MH⁺), 472.9 (MNa⁺).

### Example 199

### 6-(3-Fluoro-phenyl)-3-iodo-8-trifluoromethyl-imidazo[1,2-a]pyridine-2-carboxylic Acid(thiophen-2-ylmethyl)-amide (Compound 299)

Under standard HATU coupling conditions, 6-(3-fluoro-phenyl)-3-iodo-8-trifluoromethyl-imidazo[1,2-a]pyridine-2-carboxylic acid (compound 298) and thiophene-2-methylamine gave 6-(3-fluoro-phenyl)-3-iodo-8-trifluoromethyl-imidazo[1,2-a]pyridine-2-carboxylic acid (thiophen-2-ylmethyl)-amide (compound 299). ¹H NMR (d₆-DMSO, 300 MHz): δ 4.65 (d, 2H, J=6.0 Hz), 6.95 (dd, 1H, J=3.6, 4.8 Hz), 7.04 (m, 1H), 7.31 (m, 1H), 7.38 (dt, 1H, J=1.2, 5.1 Hz), 7.58 (m, 1H), 7.68 (d, 1H, J=7.8 Hz), 7.77 (d, 1H, J=10.2 Hz), 8.21 (s, 1H), 8.75 (s, 1H), 8.84 (t, 1H, J=6.3 Hz); MS (ESI) m/z=546 (MH⁺).

### Example 200

### 6-(3-Fluoro-phenyl)-3-propenyl-8-trifluoromethyl-imidazo[1,2-a]pyridine-2-carboxylic Acid(thiophen-2-ylmethyl)-amide (Compound 300)

3-Bromo-6-(3-fluoro-phenyl)-8-trifluoromethyl-imidazo[1,2-a]pyridine-2-carboxylic acid (thiophen-2-ylmethyl)-amide underwent Suzuki coupling with cis-1-propene-1-boronic acid to give 6-(3-fluoro-phenyl)-3-propenyl-8-trifluoromethyl-imidazo[1,2-a]pyridine-2-carboxylic acid (thiophen-2-ylmethyl)-amide (compound 300). ¹H NMR (d₆-DMSO, 300 MHz) δ 1.56 (d, 3H, J=7.2 Hz), 4.64 (d, 2H, J=6 Hz), 6.21 (dq, 1H, J=7.2, 11 Hz), 6.70 (brd, 1H, J=11 Hz), 6.94 (dt, 1H, J=0.9, 4.2 Hz), 7.02 (d, 1H, J=3 Hz), 7.27 (dt, 1H, J=2.7, 8.7 Hz), 7.36 (dt, 1H, J=1.2, 5.1 Hz), 7.54 (q, 1H, J=7.2 Hz), 7.66 (brd, 1H, J=7.5 Hz), 7.74 (brd, 1H, J=10.2 Hz), 8.14 (s, 1H), 8.55 (s, 1H), 8.74 (t, 1H, J=6 Hz); MS (ESI) m/z=460 (MH⁺).

### Example 201

### 6-(3-Fluoro-phenyl)-3-(1H-pyrazol-4-yl)-8-trifluoromethyl-imidazo[1,2-a]pyridine-2-carboxylic Acid (thiophen-2-ylmethyl)-amide (Compound 301)

6-(3-Fluoro-phenyl)-3-(1H-pyrazol-4-yl)-8-trifluoromethyl-imidazo[1,2-a]pyridine-2-carboxylic acid (thiophen-2-ylmethyl)-amide (compound 301) was prepared using Suzuki coupling as in the preparation of 6-(3-fluoro-phenyl)-3-propenyl-8-trifluoromethyl-imidazo[1,2-a]pyridine-2-carboxylic acid (thiophen-2-ylmethyl)-amide (compound 300). ¹H NMR (d₆-DMSO, 300 MHz) δ 4.62 (d, 2H, J=6.0 Hz), 6.93 (dd, 1H, J=3.6, 4.8 Hz), 6.99 (m, 1H), 7.25 (m, 1H), 7.34 (dd, 1H, J=1.2, 4.8 Hz), 7.53 (m, 1H), 7.63 (m, 1H), 7.72 (m, 1H), 8.13 (s, 1H), 8.20 (s, 2H), 8.62 (s, 1H), 8.73 (t, 1H, J=6.0 Hz); MS (ESI) m/z=486 (MH⁺).

### Example 202

### 6-(3-Fluoro-phenyl)-3-isopropenyl-8-trifluoromethyl-imidazo[1,2-a]pyridine-2-carboxylic Acid (thiophen-2-ylmethyl)-amide (Compound 302)

6-(3-Fluoro-phenyl)-3-isopropenyl-8-trifluoromethyl-imidazo[1,2-a]pyridine-2-carboxylic acid (thiophen-2-ylmethyl)-amide (compound 302) was prepared using Suzuki coupling as in the preparation of 6-(3-fluoro-phenyl)-3-propenyl-8-trifluoromethyl-imidazo[1,2-a]pyridine-2-carboxylic acid (thiophen-2-ylmethyl)-amide (compound 300). ¹H NMR (d₆-DMSO, 300 MHz) δ 1.56 (d, 3H, J=7.2 Hz), 4.62 (d, 2H, J=6.3 Hz), 5.36 (s, 1H), 5.68 (s, 1H), 7.01 (m, 1H), 7.26 (m, 1H), 7.35 (d, 1H, J=5.4 Hz), 7.54 (m, 2H), 7.71 (d, 1H, J=10.2 Hz), 8.09 (s, 1H), 8.66 (s, 1H), 8.73 (t, 1H, J=6.3 Hz); MS (ESI) m/z=460 (MH⁺).

### Example 203

### 3-Cyclohex-1-enyl-6-(3-fluoro-phenyl)-8-trifluoromethyl-imidazo[1,2-a]pyridine-2-carboxylic Acid (thiophen-2-ylmethyl)-amide (Compound 303)

3-Cyclohex-1-enyl-6-(3-fluoro-phenyl)-8-trifluoromethyl-imidazo[1,2-a]pyridine-2-carboxylic acid (thiophen-2-ylmethyl)-amide (compound 303) was prepared using Suzuki coupling as in the preparation of 6-(3-fluoro-phenyl)-3-propenyl-8-trifluoromethyl-imidazo[1, 2-a]pyridine-2-carboxylic acid (thiophen-2-ylmethyl)-amide (compound 300). ¹H NMR (d₆-DMSO, 300 MHz) δ 1.74 (m, 4H), 2.25 (m, 2H), 2.38 (m, 2H), 4.63 (d, 2H, J=6 Hz), 6.03 (brs, 1H), 6.94 (dd, 1H, J=3.3, 5.1 Hz), 7.01 (d, 1H, J=2.7 Hz), 7.28 (brt, 1H, J=8.4 Hz), 7.36 (dd, 1H, J=1.2, 4.8 Hz), 7.51-7.62 (m, 2H), 7.70 (brd, 1H, J=10 Hz), 8.06 (s, 1H), 8.61 (s, 1H), 8.66 (t, 1H, J=6 Hz); MS (ESI) m/z=500.1 (MH⁺).

### Example 204

### 3-(2-Cyclopropyl-vinyl)-6-(3-fluoro-phenyl)-8-trifluoromethyl-imidazo[1,2-a]pyridine-2-carboxylic Acid (thiophen-2-ylmethyl)-amide (Compound 304)

3-(2-Cyclopropyl-vinyl)-6-(3-fluoro-phenyl)-8-trifluoromethyl-imidazo[1,2-a]pyridine-2-carboxylic acid (thiophen-2-ylmethyl)-amide (compound 304) was prepared using Suzuki coupling as in the preparation of 6-(3-fluoro-phenyl)-3-propenyl-8-trifluoromethyl-imidazo[1,2-a]pyridine-2-carboxylic acid (thiophen-2-ylmethyl)-amide (compound 300). MS (ESI) m/z=486.1 (MH⁺).

### Example 205

### 6-(3-Fluoro-phenyl)-3-pyridin-3-ylethynyl-8-trifluoromethyl-imidazo[1,2-a]pyridine-2-carboxylic Acid (thiophen-2-ylmethyl)-amide (Compound 305)

3-Bromo-6-(3-fluoro-phenyl)-8-trifluoromethyl-imidazo[1,2-a]pyridine-2-carboxylic acid (thiophen-2-ylmethyl)-amide underwent Sonogashira coupling with 3-ethynyl-pyridine to give 6-(3-fluoro-phenyl)-3-pyridin-3-ylethynyl-8-trifluoromethyl-imidazo[1,2-a]pyridine-2-carboxylic acid (thiophen-2-ylmethyl)-amide (compound 305). ¹H NMR (d₆-DMSO, 300 MHz) δ 4.67 (d, 2H, J=5.7 Hz), 6.95 (dd, 1H, J=3.6, 5.1 Hz), 7.04 (m, 1H), 7.30 (dt, 1H, J=2.4, 8.4 Hz), 7.38 (d, 1H, J=5.1 Hz), 7.58 (m, 2H), 7.74 (d, 1H, J=7.5 Hz), 7.83 (d, 1H, J=10.2 Hz), 8.20 (d, 1H, J=7.8 Hz), 8.27 (s, 1H), 8.67 (br s, 1H), 8.95 (m, 2H), 9.08 (s, 1H); MS (ESI) m/z=521 (MH⁺).

### Example 206

### 6-(3-Fluoro-phenyl)-3-(4-hydroxy-but-1-ynyl)-8-trifluoromethyl-imidazo[1,2-a]pyridine-2-carboxylic Acid (thiophen-2-ylmethyl)-amide (Compound 306)

6-(3-Fluoro-phenyl)-3-(4-hydroxy-but-1-ynyl)-8-trifluoromethyl-imidazo[1,2-a]pyridine-2-carboxylic acid (thiophen-2-ylmethyl)-amide (compound 306) was prepared using Sonogashira coupling as in the preparation of 6-(3-fluoro-phenyl)-3-pyridin-3-ylethynyl-8-trifluoromethyl-imidazo[1,2-a]pyridine-2-carboxylic acid (thiophen-2-ylmethyl)-amide (compound 305). ¹H NMR (d₆-DMSO, 300 MHz) δ 2.80 (t, 2H, J=6.6 Hz), 3.68-3.74 (m, 2H), 4.63 (d, 2H, J=6 Hz), 5.08 (t, 1H, J=6 Hz), 6.95 (dd, 1H, J=3.3, 5.1 Hz), 7.03 (dd, 1H, J=1.2, 3.6 Hz), 7.30 (dt, 1H, J=2.4, 7.8 Hz), 7.37 (dd, 1H, J=1.2, 5.1 Hz), 7.56 (dt, 1H, J=6.3, 8.1 Hz), 7.68 (brd, 1H, J=8.4 Hz), 7.76 (dt, 1H, J=2.1, 10.2 Hz), 8.22 (brs, 1H), 8.80 (t, 1H, J=6 Hz), 8.90 (brs, 1H); MS (ESI) m/z=488 (MH⁺).

### Example 207

### 3-(3,3-Dimethyl-but-1-ynyl)-6-(3-fluoro-phenyl)-8-trifluoromethyl-imidazo[1,2-a]pyridine-2-carboxylic Acid (thiophen-2-ylmethyl)-amide (Compound 307)

3-(3,3-Dimethyl-but-1-ynyl)-6-(3-fluoro-phenyl)-8-trifluoromethyl-imidazo[1,2-a]pyridine-2-carboxylic acid (thiophen-2-ylmethyl)-amide(compound 307) was prepared using Sonogashira coupling as in the preparation of 6-(3-fluoro-phenyl)-3-pyridin-3-ylethynyl-8-trifluoromethyl-imidazo[1,2-a]pyridine-2-carboxylic acid (thiophen-2-ylmethyl)-amide(compound 305). ¹HNMR (d₆-DMSO, 300 MHz) δ 1.40 (s, 9H), 4.654 (d, 2H, J=6.3 Hz), 6.95 (dd, 1H, J=3.6, 5.1 Hz), 7.03 (dd, 1H, J=0.6, 2.1 Hz), 7.27-7.38 (m, 2H), 7.54-7.67 (m, 2H), 7.74 (brd, 1H, J=10.2 Hz), 8.21 (s, 1H), 8.66 (s, 1H), 8.76 (t, 1H, J=6 Hz); MS (ESI) m/z=500.1 (MH⁺).

### Example 208

### 3-Chloro-6-(2H-[1,2,3]triazol-4-yl)-8-trifluoromethyl-imidazo[1,2-a]pyridine-2-carboxylic Acid (thiophen-2-ylmethyl)-amide (Compound 308)

A mixture of 3-chloro-6-ethynyl-8-trifluoromethyl-imidazo[1,2-a]pyridine-2-carboxylic acid (thiophen-2-ylmethyl)-amide(compound 297) (0.129 g, 0.34 mmol), trimethylsilyl azide (0.066 mL, 0.51 mmol), copper(I) iodide (0.015 g, 0.08 mmol) in DMF (1.4 mL) and MeOH (0.15 mL) was heated at 150° C. for 18 min under microwave conditions. The product was purified by reverse phase HPLC to give 3-chloro-6-(2H-[1,2,3]triazol-4-yl)-8-trifluoromethyl-imidazo[1,2-a]pyridine-2-carboxylic acid (thiophen-2-ylmethyl)-amide(compound 308) (0.015 g, 10%). ¹H NMR (d₆-DMSO, 300 MHz) δ 4.64 (d, 2H, J=6.0 Hz), 6.95 (m, 1H), 7.03 (br d, 1H), 7.37 (d, 1H, J=5.1 Hz), 8.36 (s, 1H), 8.63 (br s, 1H), 8.92 (t, 1H, J=6.0 Hz), 9.04 (s, 1H); MS (ESI) m/z=427.0 (MH⁺).

### Example 209

### 3-Chloro-6-cyano-8-trifluoromethyl-imidazo[1,2-a]pyridine-2-carboxylic Acid (thiophen-2-ylmethyl)-amide (Compound 309)

A mixture of 6-bromo-3-chloro-8-trifluoromethyl-imidazo[1,2-a]pyridine-2-carboxylic acid (thiophen-2-ylmethyl)-amide (0.77 g, 1.76 mmol), zinc cyanide (0.3 g, 2.55 mmol), tetrakis(triphenylphosphine)palladium(0) in DMF (12 mL) was heated at 170° C. for 2 min under microwave conditions. The reaction mixture was filtered, partitioned between ethyl acetate and water. The organic layer was washed successively with saturated aqueous NaHCO₃, water, and brine. The extracts were dried (Na₂SO₄), filtered and concentrated. The product was purified by reverse phase HPLC to give 3-chloro-6-cyano-8-trifluoromethyl-imidazo[1,2-a]pyridine-2-carboxylic acid (thiophen-2-ylmethyl)-amide(compound 309) (0.1 gm, 15%). ¹H NMR (d₆-DMSO, 300 MHz) δ 4.62 (d, 2H, J=6.3 Hz), 6.93 (dd, 1H, J=3.6, 5.1 Hz), 7.01 (m, 1H), 7.35 (dd, 1H, J=1.2, 4.8 Hz), 8.30 (t, 1H, J=1.2 Hz), 8.98 (t, 1H, J=6.3 Hz), 9.58 (s, 1H); MS (ESI) m/z=385 (MH⁺).

### Example 210

### 3-Chloro-6-(5-oxo-4,5-dihydro-[1,2,4]oxadiazol-3-yl)-8-trifluoromethyl-imidazo[1,2-a]pyridine-2-carboxylic Acid (thiophen-2-ylmethyl)-amide (Compound 310)

### Step 1: 3-Chloro-6-(N-hydroxycarbamimidoyl)-8-trifluoromethyl-imidazo[1,2-a]pyridine-2-carboxylic Acid (thiophen-2-ylmethyl)-amide

3-Chloro-6-(N-hydroxycarbamimidoyl)-8-trifluoromethyl-imidazo[1,2-a]pyridine-2-carboxylic acid (thiophen-2-ylmethyl)-amide was prepared by treating 3-chloro-6-cyano-8-trifluoromethyl-imidazo[1,2-a]pyridine-2-carboxylic acid (thiophen-2-ylmethyl)-amide (compound 309) with hydroxylamine in EtOH followed by reverse phase HPLC purification. MS (ESI) m/z=418.0 (MH⁺).

### Step 2: 3-Chloro-6-(5-oxo-4,5-dihydro-[1,2,4]oxadiazol-3-yl)-8-trifluoromethyl-imidazo[1,2-a]pyridine-2-carboxylic Acid (thiophen-2-ylmethyl)-amide (compound 310)

A mixture of 3-chloro-6-(N-hydroxycarbamimidoyl)-8-trifluoromethyl-imidazo[1,2-a]pyridine-2-carboxylic acid (thiophen-2-ylmethyl)-amide (Example 210, step 1) (0.12 g, 0.29 mmol), carbonyldiimidazole (0.056 g, 0.34 mmol) and 1,4-dioxane (10 mL) was heated at 70° C. for 2 h followed by heating at 100° C. for 3 h. After aqueous workup, the crude material was purified by reverse phase HPLC to afford 3-chloro-6-(5-oxo-4,5-dihydro-[1,2,4]oxadiazol-3-yl)-8-trifluoromethyl-imidazo[1,2-a]pyridine-2-carboxylic acid (thiophen-2-ylmethyl)-amide (compound 310) (0.02 g, 16%). MS (ESI) m/z=443.9 (MH⁺).

### Example 211

### 3-Chloro-6-[1,2,4]oxadiazol-3-yl-8-trifluoromethyl-imidazo[1,2-a]pyridine-2-carboxylic Acid (thiophen-2-ylmethyl)-amide (Compound 311)

To a solution of 3-chloro-6-(N-hydroxycarbamimidoyl)-8-trifluoromethyl-imidazo[1,2-a]pyridine-2-carboxylic acid (thiophen-2-ylmethyl)-amide (Example 210, Step 1) (0.1 g, 0.2 mmol) in trimethylorthoformate (15 mL) was added 2 drops of boron trifluoride etherate. The mixture was then heated at 110° C. for 30 min. After aqueous workup, the product was purified by reverse phase HPLC to afford 3-chloro-6-[1,2,4]oxadiazol-3-yl-8-trifluoromethyl-imidazo[1,2-a]pyridine-2-carboxylic acid (thiophen-2-ylmethyl)-amide (compound 311) (0.015 g, 18%). ¹H NMR (d₆-DMSO, 300 MHz) δ 4.65 (d, 2H, J=6.0 Hz), 6.95 (m, 1H), 7.03 (m, 1H), 7.37 (d, 1H, J=5.1 Hz), 8.25 (s, 1H), 8.99 (t, 1H, J=6.0 Hz), 9.04 (s, 1H), 9.89 (s, 1H); MS (ESI) m/z=428.0 (MH⁺), 450 (MNa⁺).

### Example 212

### 3-Chloro-2-[(thiophen-2-ylmethyl)-carbamoyl]-8-trifluoromethyl-imidazo[1,2-a]pyridine-6-carboxylic Acid Methyl Ester (Compound 312)

Hydrogen chloride gas was bubbled to a solution of 3-chloro-6-cyano-8-trifluoromethyl-imidazo[1,2-a]pyridine-2-carboxylic acid (thiophen-2-ylmethyl)-amide (compound 309) (0.38 g, 0.99 mmol) in MeOH (100 mL) at 0° C. for 15 minutes. The flask was sealed and allowed to warm to room temperature. After 18 hours, water was added to the mixture followed by the removel of MeOH. After aqueous workup, 3-chloro-2-[(thiophen-2-ylmethyl)-carbamoyl]-8-trifluoromethyl-imidazo[1,2-a]pyridine-6-carboxylic acid methyl ester (compound 312) (0.2 gm, 48%) was obtained. ¹H NMR (d₆-DMSO, 300 MHz) δ 3.95 (s, 3H), 4.63 (d, 2H, J=6.0 Hz), 6.94 (m, 1H), 7.02 (m, 1H), 7.36 (dd, 1H, J=1.2, 4.8 Hz), 8.10 (s, 1H), 8.98 (br m, 2H); MS (ESI) m/z=417.9 (MH⁺), 439.9 (MNa⁺).

### Example 213

### 3-Chloro-2-[(thiophen-2-ylmethyl)-carbamoyl]-8-trifluoromethyl-imidazo[1,2-a]pyridine-6-carboxylic Acid (Compound 313)

To a solution of 3-chloro-2-[(thiophen-2-ylmethyl)-carbamoyl]-8-trifluoromethyl-imidazo[1,2-a]pyridine-6-carboxylic acid methyl ester (compound 312) (0.14 g, 0.33 mmol) in THF (4.5 mL) and water (1.5 mL), LiOH (0.042 g, 1 mmol) was added. The mixture was stirred for 1 hour followed by the removal of solvent under reduced pressure. The crude material was purified by reverse phase HPLC to afford 3-chloro-2-[(thiophen-2-ylmethyl)-carbamoyl]-8-trifluoromethyl-imidazo[1,2-a]pyridine-6-carboxylic acid (compound 313) (0.015 g, 11%). MS (ESI) m/z=404.0 (MH⁺).

### Example 214

### 6-(3-Fluoro-phenyl)-8-trifluoromethyl-imidazo[1,2-a]pyridine-2-carboxylic Acid (thiophen-2-ylmethyl)-amide (Compound 314)

6-(3-Fluoro-phenyl)-8-trifluoromethyl-imidazo[1,2-a]pyridine-2-carboxylic acid (thiophen-2-ylmethyl)-amide (compound 314) was obtained as a major side product from a palladium reaction (using Pd₂ (dba₃)₄ as a catalyst) of 6-(3-fluoro-phenyl)-3-iodo-8-trifluoromethyl-imidazo[1,2-a]pyridine-2-carboxylic acid (thiophen-2-ylmethyl)-amide (compound 299). ¹H NMR (d₆-DMSO, 300 MHz) δ 4.65 (d, 2H, J=6.6 Hz), 6.96 (m, 1H), 7.03 (m, 1H), 7.29 (br t, 1H), 7.37 (dd, 1H, J=5.1, 1.2 Hz), 7.61 (m, 3H), 8.14 (s, 1H), 8.51 (s, 1H), 8.85 (t, 1H, J=6.6 Hz), 9.28 (s, 1H); MS (ESI) m/z=420.0 (MH⁺).

### Example 215

### 3-Chloro-6-(2H-tetrazol-5-yl)-8-trifluoromethyl-imidazo[1,2-a]pyridine-2-carboxylic Acid(thiophen-2-ylmethyl)-amide (Compound 315)

A mixture of 6-bromo-3-chloro-8-trifluoromethyl-imidazo[1,2-a]pyridine-2-carboxylic acid (thiophen-2-ylmethyl)-amide (0.1 g, 0.23 mmol), zinc cyanide (0.032 g, 0.27 mmol), and tetrakis(triphenylphosphine)palladium(0) (0.014 g, 0.01 mmol) were heated in DMF at 170° C. for 4 minutes under microwave conditions. Sodium azide (0.21 g, 3.24 mmol) and ammonium chloride (0.17 g, 3.24 mmol) were then added and the mixture heated again at 170° C. for 5 minutes under microwave conditions. After aqueous workup, the product was purified by reverse phase HPLC to afford 3-chloro-6-(2H-tetrazol-5-yl)-8-trifluoromethyl-imidazo[1,2-a]pyridine-2-carboxylic acid (thiophen-2-ylmethyl)-amide (compound 315) (0.015 g, 15%). MS (ESI) m/z=428.0 (MH⁺).

### Example 216

### (3-Chloro-6-furan-2-yl-8-trifluoromethyl-imidazo[1,2-a]pyridin-2-yl)-[3-(4-fluoro-phenyl)-pyrrolidin-1-yl]-methanone (Compound 316)

Using standard HATU coupling conditions, 3-chloro-6-furan-2-yl-8-trifluoromethyl-imidazo[1,2-a]pyridine-2-carboxylic acid, and 3-(4-fluorophenyl)pyrrolidine gave (3-chloro-6-furan-2-yl-8-trifluoromethyl-imidazo[1,2-a]pyridin-2-yl)-[3-(4-fluoro-phenyl)-pyrrolidin-1-yl]-methanone (compound 316). ¹H NMR (d₆-DMSO): δ 2.05 (m, 1H), 2.28 (m, 1H), 3.57-3.81 (m, 3.5H), 4.03 (m, 1H), 4.24 (0.5H), 6.68 (m, 1H), 7.13 (q, 2H, J=8.4 Hz), 7.36 (m, 3H), 7.86 (m, 1H), 8.20 (s, 0.5H), 8.22 (s, 0.5H), 8.68 (s, 0.5H), 8.70 (s, 0.5H); MS (ESI) m/z=478.1 (MH⁺).

### Example 217

### (3-Chloro-6-furan-2-yl-8-trifluoromethyl-imidazo[1,2-a]pyridin-2-yl)-(3-hydroxy-3-phenyl-pyrrolidin-1-yl)-methanone (Compound 317)

Using standard HATU coupling conditions, 3-chloro-6-furan-2-yl-8-trifluoromethyl-imidazo[1,2-a]pyridine-2-carboxylic acid, and 3-phenyl-pyrrolidin-3-ol gave (3-chloro-6-furan-2-yl-8-trifluoromethyl-imidazo[1,2-a]pyridin-2-yl)-(3-hydroxy-3-phenyl-pyrrolidin-1-yl)-methanone (compound 317). ¹H NMR (d₆-DMSO): δ 2.14 (m, 1H), 2.34 (m, 1H), 3.48 (brs, 1H), 3.65-4.11 (m, 4H), 6.68 (m, 1H), 7.30 (m, 4H), 7.55 (m, 2H), 7.86 (m, 1H), 8.19 (s, 0.5H), 8.22 (s, 0.5H), 8.67 (s, 0.5H), 8.70 (s, 0.5H); MS (ESI) m/z=476.1 (MH⁺);

### Example 218

### (3-Chloro-6-furan-2-yl-8-trifluoromethyl-imidazo[1,2-a]pyridin-2-yl)-(4-methyl-3-phenyl-piperazin-1-yl)-methanone (Compound 318)

Using standard HATU coupling conditions, 3-chloro-6-furan-2-yl-8-trifluoromethyl-imidazo[1,2-a]pyridine-2-carboxylic acid, and 1-methyl-2-phenyl-piperazine gave (3-chloro-6-furan-2-yl-8-trifluoromethyl-imidazo[1,2-a]pyridin-2-yl)-(4-methyl-3-phenyl-piperazin-1-yl)-methanone (compound 318). ¹H NMR (d₆-DMSO): δ 2.49 (m, 1H), 2.60 (brs, 3H), 3.37 (m, 2H), 3.72 (m, 2H), 4.57 (m, 2H), 4.78 (d, 2H, J=12 Hz), 6.66 (brs, 1H), 7.33-7.59 (m, 6H), 7.63 (s, 0.5H), 7.86 (s, 0.5H), 8.18 (s, 0.5H), 8.26 (s, 0.5H), 8.65 (s, 0.5H), 8.71 (s, 0.5H); MS (ESI) m/z=489.1 (MH⁺).

### Example 219

### 3-Chloro-6-furan-2-yl-8-trifluoromethyl-imidazo[1,2-a]pyridine-2-carboxylic Acid (2-dimethylamino-ethyl)-thiophen-2-ylmethyl-amide (Compound 319)

Using standard HATU coupling conditions, 3-chloro-6-furan-2-yl-8-trifluoromethyl-imidazo[1,2-a]pyridine-2-carboxylic acid, and N,N-dimethyl-N′-thiophen-2-ylmethyl-ethane-1,2-diamine gave 3-chloro-6-furan-2-yl-8-trifluoromethyl-imidazo[1,2-a]pyridine-2-carboxylic acid (2-dimethylamino-ethyl)-thiophen-2-ylmethyl-amide (compound 319). ¹H NMR (d₆-DMSO): δ 2.75 (s, 3H), 2.86 (s, 3H), 3.33 (m, 1H), 3.54 (m, 1H), 3.74 (m, 1H), 3.81 (m, 1H), 4.84 (s, 1H), 5.23 (s, 1H), 6.68 (dd, 1H, J=1.8, 3.6 Hz), 6.97 (ddd, 1H, J=3.2, 4.8, 9.9 Hz), 7.13 (dd, 1H, J=2.4, 19.2 Hz), 7.38 (d, 1H, J=3.6 Hz), 7.47 (dd, 1H, J=5.4, 7.5 Hz), 7.86 (d, 1H, J=1.5 Hz), 8.25 (s, 1H), 8.71 (s, 1H); MS (ESI) m/z=497.1 (MH⁺).

### Example 220

### (3-Chloro-6-furan-2-yl-8-trifluoromethyl-imidazo[1,2-a]pyridin-2-yl)-(4-methyl-2-phenyl-piperazin-1-yl)-methanone (Compound 320)

Using standard HATU coupling conditions, 3-chloro-6-furan-2-yl-8-trifluoromethyl-imidazo[1,2-a]pyridine-2-carboxylic acid, and 1-methyl-3-phenyl-piperazine gave (3-chloro-6-furan-2-yl-8-trifluoromethyl-imidazo[1,2-a]pyridin-2-yl)-(4-methyl-2-phenyl-piperazin-1-yl)-methanone (compound 320). ¹H NMR (d₆-DMSO): δ 2.87 (s, 3H), 3.36 (m, 4H), 4.34 (d, 1H, J=14 Hz), 4.67 (m, 1H), 6.15 (brs, 1H), 6.69 (brs, 1H), 7.44 (m, 6H), 7.86 (brs, 1H), 8.18 (s, 0.5H), 8.28 (s, 0.5H), 8.67 (s, 0.5H), 8.74 (s, 0.5H); MS (ESI) m/z=489.1 (MH⁺).

### Example 221

### 3-Chloro-6-furan-2-yl-8-trifluoromethyl-imidazo[1,2-a]pyridine-2-carboxylic Acid phenethyl-amide (Compound 321)

Using standard HATU coupling conditions, 3-chloro-6-furan-2-yl-8-trifluoromethyl-imidazo[1,2-a]pyridine-2-carboxylic acid, and phenethylamine gave 3-chloro-6-furan-2-yl-8-trifluoromethyl-imidazo[1,2-a]pyridine-2-carboxylic acid phenethyl-amide (compound 321). ¹H NMR (d₆-DMSO): δ 2.85 (m, 2H), 3.51 (m, 2H), 6.68 (m, 1H), 7.24 (m, 5H), 7.31 (d, 1H, J=3 Hz), 7.85 (d, 1H, J=10 Hz), 8.21 (d, 1H), 8.28 (t, 1H, J=6 Hz), 8.65 (s, 1H), MS (ESI) m/z=434.1 (MH⁺).

### Example 222

### (3-Chloro-6-furan-2-yl-8-trifluoromethyl-imidazo[1,2-a]pyridin-2-yl)-(2-phenyl-pyrrolidin-1-yl)-methanone (Compound 322)

Using standard HATU coupling conditions, 3-chloro-6-furan-2-yl-8-trifluoromethyl-imidazo[1,2-a]pyridine-2-carboxylic acid, and 2-phenylpyrrolidine gave (3-chloro-6-furan-2-yl-8-trifluoromethyl-imidazo[1,2-a]pyridin-2-yl)-(2-phenyl-pyrrolidin-1-yl)-methanone (compound 322). ¹H NMR (d₆-DMSO): δ 1.85 (m, 3H), 2.39 (m, 1H), 3.85 (m, 1H), 4.11 (m, 1H), 5.23 (m, 0.5H), 5.66 (m, 0.5H), 6.67 (m, 1H), 6.95 (m, 3H), 7.29 (m, 3H), 7.82 (brs, 0.5H), 7.85 (brs, 0.5H), 8.13 (s, 0.5H), 8.22 (s, 0.5H), 8.44 (s, 0.5H), 8.68 (s, 0.5H); MS (ESI) m/z=460.1 (MH⁺);

### Example 223

### (3-Chloro-6-furan-2-yl-8-trifluoromethyl-imidazo[1,2-a]pyridin-2-yl)-(4-phenyl-piperazin-1-yl)-methanone (Compound 323)

Using standard HATU coupling conditions, 3-chloro-6-furan-2-yl-8-trifluoromethyl-imidazo[1,2-a]pyridine-2-carboxylic acid, and 1-phenyl piperazine gave (3-chloro-6-furan-2-yl-8-trifluoromethyl-imidazo[1,2-a]pyridin-2-yl)-(4-phenyl-piperazin-1-yl)-methanone (compound 323). ¹H NMR (d₆-DMSO): δ 3.17 (m, 2H), 3.24 (m, 2H), 3.76 (m, 4H), 6.68 (m, 1H), 6.81 (t, 1H, J=7.8 Hz), 6.96 (m, 2H), 7.21 (m, 2H), 7.37 (d, 1H, J=3.6 Hz), 7.86 (d, 1H, J=3 Hz), 8.22 (s, 1H), 8.69 (s, 1H); MS (ESI) m/z=475.1 (MH⁺).

### Example 224

### (4-Benzyl-piperazin-1-yl)-(3-chloro-6-furan-2-yl-8-trifluoromethyl-imidazo[1,2-a]pyridin-2-yl)-methanone (Compound 324)

Using standard HATU coupling conditions, 3-chloro-6-furan-2-yl-8-trifluoromethyl-imidazo[1,2-a]pyridine-2-carboxylic acid, and 1-benzyl piperazine gave (4-benzyl-piperazin-1-yl)-(3-chloro-6-furan-2-yl-8-trifluoromethyl-imidazo[1,2-a]pyridin-2-yl)-methanone (compound 324). ¹H NMR (d₆-DMSO): δ 3.55 (m, 8H), 4.60 (s, 2H), 6.67 (m, 1H), 7.25 (d, 1H, J=3 Hz), 7.47 (m, 3H), 7.63 (m, 2H), 7.77 (d, 1H, J=3 Hz), 8.19 (m, 1H), 8.74 (s, 1H); MS (ESI) m/z=489.1 (MH⁺).

### Example 225

### 3-Chloro-6-furan-2-yl-8-trifluoromethyl-imidazo[1,2-a]pyridine-2-carboxylic Acid (1-methyl-1H-imidazol-4-ylmethyl)-amide (Compound 325)

Using standard HATU coupling conditions, 3-chloro-6-furan-2-yl-8-trifluoromethyl-imidazo[1,2-a]pyridine-2-carboxylic acid, and C-(1-methyl-1H-imidazol-4-yl)-methylamine gave 3-chloro-6-furan-2-yl-8-trifluoromethyl-imidazo[1,2-a]pyridine-2-carboxylic acid (1-methyl-1H-imidazol-4-ylmethyl)-amide (compound 325). ¹H NMR (d₆-DMSO): δ 3.80 (s, 3H), 4.50 (d, 2H, J=6.3 Hz), 6.69 (m, 1H), 7.39 (d, 1H, J=3.6 Hz), 7.52 (s, 1H), 7.87 (d, 1H, J=1.8 Hz), 8.26 (s, 1H), 8.69 (s, 1H), 8.82 (m, 2H); MS (ESI) m/z=424.0 (MH⁺).

### Example 226

### (3-Benzyl-pyrrolidin-1-yl)-(3-chloro-6-furan-2-yl-8-trifluoromethyl-imidazo[1,2-a]pyridin-2-yl)-methanone (Compound 326)

Using standard HATU coupling conditions, 3-chloro-6-furan-2-yl-8-trifluoromethyl-imidazo[1,2-a]pyridine-2-carboxylic acid, and 3-benzyl-pyrrolidine gave (3-benzyl-pyrrolidin-1-yl)-(3-chloro-6-furan-2-yl-8-trifluoromethyl-imidazo[1,2-a]pyridin-2-yl)-methanone (compound 326). ¹H NMR (d₆-DMSO): δ 1.61 (m, 1H), 1.95 (m, 1H), 2.65 (m, 2H), 3.50-3.87 (m, 4.5H), 8.66 (s, 0.5H), 6.66 (m, 1H), 7.22 (m, 6H), 7.84 (brs, 1H), 8.18 (brs, 1H), 8.64 (s, 0.5H); MS (ESI) m/z=474.1 (MH⁺).

### Example 227

### 3-Chloro-6-furan-2-yl-8-trifluoromethyl-imidazo[1,2-a]pyridine-2-carboxylic Acid (3-methyl-3H-imidazol-4-ylmethyl)-amide (Compound 327)

Using standard HATU coupling conditions, 3-chloro-6-furan-2-yl-8-trifluoromethyl-imidazo[1,2-a]pyridine-2-carboxylic acid, and C-(3-methyl-3H-imidazol-4-yl)-methylamine gave 3-chloro-6-furan-2-yl-8-trifluoromethyl-imidazo[1,2-a]pyridine-2-carboxylic acid (3-methyl-3H-imidazol-4-ylmethyl)-amide (compound 327). ¹H NMR (d₆-DMSO): δ 3.89 (s, 3H), 4.56 (d, 2H, J=6 Hz), 6.67 (m, 1H), 7.37 (d, 1H, J=3.3 Hz), 7.54 (brs, 1H), 7.85 (s, 1H), 8.24 (s, 1H), 8.68 (s, 1H), 8.91 (t, 1H, J=6 Hz), 8.98 (s, 1H); MS (ESI) m/z=424.0 (MH⁺).

### Example 228

### (3-Benzyl-azetidin-1-yl)-(3-chloro-6-furan-2-yl-8-trifluoromethyl-imidazo[1,2-a]pyridin-2-yl)-methanone (Compound 328)

Using standard HATU coupling conditions, 3-chloro-6-furan-2-yl-8-trifluoromethyl-imidazo[1,2-a]pyridine-2-carboxylic acid, and 3-benzyl-azetidine gave (3-benzyl-azetidin-1-yl)-(3-chloro-6-furan-2-yl-8-trifluoromethyl-imidazo[1,2-a]pyridin-2-yl)-methanone (compound 328). ¹H NMR (d₆-DMSO): δ 2.94 (m, 3.5H), 3.77 (m, 0.5H), 4.05-4.30 (m, 2H), 4.61 (t, 1H, J=8 Hz), 6.67 (m, 1H), 7.24 (m, 5H), 7.36 (d, 1H, J=3.3 Hz), 7.86 (d, 1H, J=1.8 Hz), 8.21 (s, 1H), 8.67 (s, 1H); MS (ESI) m/z=460.1 (MH⁺).

### Example 229

### (3-Chloro-6-furan-2-yl-8-trifluoromethyl-imidazo[1,2-a]pyridin-2-yl)-[2-(4-fluoro-phenyl)-pyrrolidin-1-yl]-methanone (Compound 329)

Using standard HATU coupling conditions, 3-chloro-6-furan-2-yl-8-trifluoromethyl-imidazo[1,2-a]pyridine-2-carboxylic acid, and 2-(4-fluorophenyl)pyrrolidine gave (3-chloro-6-furan-2-yl-8-trifluoromethyl-imidazo[1,2-a]pyridin-2-yl)-[2-(4-fluoro-phenyl)-pyrrolidin-1-yl]-methanone (compound 329). ¹H NMR (d₆-DMSO): δ 1.78 (m, 1H), 1.90 (m, 1H), 2.38 (m, 1H), 3.81-4.11 (m, 3H), 8.67 (s, 0.5H), 5.21 (m, 0.5H), 5.65 (m, 0.5H), 6.66 (m, 1H), 6.91 (m, 2H), 7.14 (m, 1H), 7.28 (m, 1H), 7.36 (d, 1H, J=3 Hz), 7.82 (brs, 0.5H), 7.85 (brs, 0.5H), 8.13 (s, 0.5H), 8.22 (s, 0.5H), 8.49 (s, 0.5H); MS (ESI) m/z=478.1 (MH⁺).

### Example 230

### (3-Chloro-6-furan-3-yl-8-trifluoromethyl-imidazo[1,2-a]pyridin-2-yl)-(2,2-dimethyl-pyrrolidin-1-yl)-methanone (Compound 330)

Using standard HATU coupling conditions, 3-chloro-6-furan-3-yl-8-trifluoromethyl-imidazo[1,2-a]pyridine-2-carboxylic acid, and 2,2,-dimethylpyrrolidine gave (3-chloro-6-furan-3-yl-8-trifluoromethyl-imidazo[1,2-a]pyridin-2-yl)-(2,2-dimethyl-pyrrolidin-1-yl)-methanone (compound 330). ¹H NMR (d₆-DMSO): δ 1.59 (s, 6H), 1.87 (m, 4H), 3.81 (t, 2H, J=7 Hz), 7.18 (m, 1H), 7.74 (t, 1H, J=1.8 Hz), 8.09 (brs, 1H), 8.37 (s, 1H), 8.73 (s, 1H); MS (ESI) m/z=412.1 (MH⁺).

### Example 231

### (3-Chloro-6-furan-3-yl-8-trifluoromethyl-imidazo[1,2-a]pyridin-2-yl)-(2-pyridin-2-yl-pyrrolidin-1-yl)-methanone (Compound 331)

Using standard HATU coupling conditions, 3-chloro-6-furan-3-yl-8-trifluoromethyl-imidazo[1,2-a]pyridine-2-carboxylic acid, and 2-pyrrolidin-2-yl-pyridine gave (3-chloro-6-furan-3-yl-8-trifluoromethyl-imidazo[1,2-a]pyridin-2-yl)-(2-pyridin-2-yl-pyrrolidin-1-yl)-methanone (compound 331). ¹H NMR (d₆-DMSO): δ 1.90 (m, 1H), 2.06 (m, 1H), 2.13 (m, 1H), 2.57 (m, 1H), 3.93 (m, 1H), 4.27 (m, 0.5H), 4.41 (m, 0.5H), 5.55 (m, 0.5H), 6.16 (d, 0.5H, J=7.8 Hz), 7.13 (m, 0.5H), 7.19 (m, 0.5H), 7.73 (m, 2H), 7.81 (d, 1H, J=7.8 Hz), 8.01 (s, 0.5H), 8.15 (s, 0.5H), 8.24 (m, 1H), 8.33 (s, 0.5H), 8.39 (s, 0.5H), 8.67 (s, 0.5H), 8.76 (s, 0.5H), 8.82 (d, 1H, J=4.5 Hz); MS (ESI) m/z=461.1 (MH⁺).

### Example 232

### 3-Chloro-6-furan-3-yl-8-trifluoromethyl-imidazo[1,2-a]pyridine-2-carboxylic Acid Methyl-thiophen-2-ylmethyl-amide (Compound 332)

Using standard HATU coupling conditions, 3-chloro-6-furan-3-yl-8-trifluoromethyl-imidazo[1,2-a]pyridine-2-carboxylic acid, and methyl-thiophen-2-ylmethyl-amine gave 3-chloro-6-furan-3-yl-8-trifluoromethyl-imidazo[1,2-a]pyridine-2-carboxylic acid methyl-thiophen-2-ylmethyl-amide (compound 332). ¹H NMR (d₆-DMSO): δ 3.05 (s, 1.5H), 3.26 (s, 1.5H), 4.93 (s, 1H), 5.21 (s, 1H), 6.97 (m, 1H), 7.14 (m, 2H), 7.37 (m, 1H), 7.75 (s, 1H), 8.12 (brs, 1H), 8.38 (s, 1H), 8.76 (brs, 1H); MS (ESI) m/z=440.0 (MH⁺).

### Example 233

### (3-Chloro-6-furan-3-yl-8-trifluoromethyl-imidazo[1,2-a]pyridin-2-yl)-[3-(2-fluoro-phenyl)-pyrrolidin-1-yl]-methanone (Compound 333)

Using standard HATU coupling conditions, 3-chloro-6-furan-3-yl-8-trifluoromethyl-imidazo[1,2-a]pyridine-2-carboxylic acid, and 3-(2-fluorophenyl)pyrrolidine gave (3-chloro-6-furan-3-yl-8-trifluoromethyl-imidazo[1,2-a]pyridin-2-yl)-[3-(2-fluoro-phenyl)-pyrrolidin-1-yl]-methanone (compound 333). ¹H NMR (d₆-DMSO): δ 2.11 (m, 1H), 2.29 (m, 1H), 3.49 (m, 1H), 3.63 (m, 1H), 3.80 (m, 2H), 4.04 (m, 0.5H), 4.27 (m, 0.5H), 7.21 (m, 2H), 7.30 (m, 2H), 7.41 (m, 1H), 7.82 (m, 1H), 8.16 (s, 0.5H), 8.19 (s, 0.5H), 8.53 (s, 0.5H), 8.54 (s, 0.5H), 8.79 (s, 0.5H), 8.81 (s, 0.5H); MS (ESI) m/z=478.1 (MH⁺).

### Example 234

### (3-Chloro-6-furan-3-yl-8-trifluoromethyl-imidazo[1,2-a]pyridin-2-yl)-[3-(3-fluoro-phenyl)-pyrrolidin-1-yl]-methanone (Compound 334)

Using standard HATU coupling conditions, 3-chloro-6-furan-3-yl-8-trifluoromethyl-imidazo[1,2-a]pyridine-2-carboxylic acid, and 3-(3-fluorophenyl)pyrrolidine gave (3-chloro-6-furan-3-yl-8-trifluoromethyl-imidazo[1,2-a]pyridin-2-yl)-[3-(3-fluoro-phenyl)-pyrrolidin-1-yl]-methanone (compound 334). ¹H NMR (d₆-DMSO): δ 2.06 (m, 1H), 2.29 (m, 1H), 3.51 (m, 2H), 3.76 (m, 1H), 3.85 (m, 0.5H), 4.05 (m, 1H), 4.24 (m, 0.5H), 7.05 (m, 1H), 7.16 (m, 2H), 7.33 (m, 2H), 7.81 (m, 1H), 8.16 (s, 0.5H), 8.19 (s, 0.5H), 8.53 (s, 0.5H), 8.54 (s, 0.5H), 8.80 (s, 0.5H), 8.81 (s, 0.5H); MS (ESI) m/z=478.1 (MH⁺).

### Example 235

### (3-Chloro-6-furan-3-yl-8-trifluoromethyl-imidazo[1,2-a]pyridin-2-yl)-[3-(4-methoxy-phenyl)-pyrrolidin-1-yl]-methanone (Compound 335)

Using standard HATU coupling conditions, 3-chloro-6-furan-3-yl-8-trifluoromethyl-imidazo[1,2-a]pyridine-2-carboxylic acid, and 3-(4-methoxyphenyl)pyrrolidine gave (3-chloro-6-furan-3-yl-8-trifluoromethyl-imidazo[1,2-a]pyridin-2-yl)-[3-(4-methoxy-phenyl)-pyrrolidin-1-yl]-methanone (compound 335). ¹H NMR (d₆-DMSO): δ 2.01 (m, 1H), 2.24 (m, 1H), 3.38 (m, 2H), 3.59 (m, 1H), 3.70 (brs, 1.5H), 3.72 (brs, 1.5H), 3.82 (m, 0.5H), 4.02 (m, 1H), 4.20 (m, 0.5H), 6.87 (t, 2H, J=8.4 Hz), 7.28 (m, 3H), 7.82 (m, 1H), 8.16 (brs, 1H), 8.54 (brs, 0.5H), 8.79 (s, 0.5H), 8.80 (s, 0.5H); MS (ESI) m/z=490.1 (MH⁺).

### Example 236

### (3-Chloro-6-furan-3-yl-8-trifluoromethyl-imidazo[1,2-a]pyridin-2-yl)-[3-(4-trifluoromethyl-phenyl)-pyrrolidin-1-yl]-methanon (Compound 336)

Using standard HATU coupling conditions, 3-chloro-6-furan-3-yl-8-trifluoromethyl-imidazo[1,2-a]pyridine-2-carboxylic acid, and 3-(4-trifluoromethyl-phenyl)pyrrolidine gave (3-chloro-6-furan-3-yl-8-trifluoromethyl-imidazo[1,2-a]pyridin-2-yl)-[3-(4-trifluoromethyl-phenyl)-pyrrolidin-1-yl]-methanone (compound 336). ¹H NMR (d₆-DMSO): δ 2.09 (m, 1H), 2.34 (m, 1H), 3.54 (m, 2H), 3.76 (m, 1H), 3.89 (m, 0.5H), 4.08 (m, 1H), 4.28 (m, 0.5H), 7.31 (m, 1H), 7.56 (m, 2H), 7.68 (m, 2H), 7.81 (m, 1H), 8.16 (s, 0.5H), 8.19 (s, 0.5H), 8.53 (s, 0.5H), 8.55 (s, 0.5H), 8.79 (s, 0.5H), 8.81 (s, 0.5H); MS (ESI) m/z=528.1 (MH⁺).

### Example 237

### [3-(2-Fluoro-phenyl)-pyrrolidin-1-yl]-(6-furan-3-yl-8-trifluoromethyl-imidazo[1,2-a]pyridin-2-yl)-methanone (Compound 337)

Using standard HATU coupling conditions, 6-furan-3-yl-8-trifluoromethyl-imidazo[1,2-a]pyridine-2-carboxylic acid, and 3-(2-fluorophenyl)pyrrolidine gave [3-(2-fluoro-phenyl)-pyrrolidin-1-yl]-(6-furan-3-yl-8-trifluoromethyl-imidazo[1,2-a]pyridin-2-yl)-methanone (compound 337). ¹H NMR (d₆-DMSO): δ 2.12 (m, 1H), 2.29 (m, 1H), 3.55 (m, 1H), 3.77 (m, 1H), 3.92 (m, 1H), 4.03 (m, 1H), 4.32 (m, 0.5H), 4.55 (q, 0.5H, J=4 Hz), 7.00 (m, 1H), 7.19 (m, 2H), 7.28 (m, 1H), 7.40 (t, 1H, J=9 Hz), 7.81 (m, 1H), 8.05 (s, 0.5H), 8.08 (s, 0.5H), 8.41 (d, 2H, J=2.4 Hz), 9.11 (s, 0.5H), 9.13 (s, 0.5H); MS (ESI) m/z=444.1 (MH⁺).

### Example 238

### 2-[1-(3-Chloro-6-furan-3-yl-8-trifluoromethyl-imidazo[1,2-a]pyridine-2-carbonyl)-pyrrolidin-3-yl]-benzoic Acid Methyl Ester (Compound 338)

Using standard HATU coupling conditions, 3-chloro-6-furan-3-yl-8-trifluoromethyl-imidazo[1,2-a]pyridine-2-carboxylic acid, and 2-pyrrolidin-3-yl-benzoic acid methyl ester gave 2-[1-(3-chloro-6-furan-3-yl-8-trifluoromethyl-imidazo[1,2-a]pyridine-2-carbonyl)-pyrrolidin⁻³-yl]-benzoic acid methyl ester (compound 338). ¹H NMR (d₆-DMSO): δ 2.21 (m, 2H), 3.48 (m, 1H), 3.59 (m, 1H), 3.80 (d, 1.5H, J=1.8 Hz), 3.85 (d, 1.5H, J=1.8 Hz), 4.02 (m, 2H), 4.24 (m, 1H), 7.37-7.29 (m, 2H), 7.57 (m, 2H), 7.71 (m, 1H), 7.81 (m, 1H), 8.14 (s, 0.5H), 8.18 (s, 0.5H), 8.51 (s, 0.5H), 8.54 (s, 0.5H), 8.78 (s, 0.5H), 8.80 (s, 0.5H); MS (ESI) m/z=518.1 (MH⁺).

### Example 239

### (3-Chloro-6-furan-3-yl-8-trifluoromethyl-imidazo[1,2-a]pyridin-2-yl)-[3-(3,4-dimethoxy-phenyl)-pyrrolidin-1-yl]-methanone (Compound 339)

Using standard HATU coupling conditions, 3-chloro-6-furan-3-yl-8-trifluoromethyl-imidazo[1,2-a]pyridine-2-carboxylic acid, and 3-(3,4-dimethoxy-phenyl)-pyrrolidine gave (3-chloro-6-furan-3-yl-8-trifluoromethyl-imidazo[1,2-a]pyridin-2-yl)-[3-(3,4-dimethoxy-phenyl)-pyrrolidin-1-yl]-methanone (compound 339). ¹H NMR (d₆-DMSO): δ 2.03 (m, 1H), 2.25 (m, 1H), 3.37 (m, 2H), 3.56 (m, 0.5H), 3.71 (m, 6H), 4.01 (m, 2H), 4.23 (m, 0.5H), 6.87 (m, 3H), 7.29 (m, 1H), 7.81 (m, 1H), 8.16 (d, 0.5H, 0.9 Hz), 8.18 (d, 0.5H, J=0.9 Hz), 8.52 (d, 0.5H, J=0.9 Hz), 8.57 (d, 0.5H, J=0.9 Hz), 8.78 (s, 0.5H), 8.80 (s, 0.5H); MS (ESI) m/z=520.1 (MH⁺).

### Example 240

### (3-Chloro-6-furan-3-yl-8-trifluoromethyl-imidazo[1,2-a]pyridin-2-yl)-(3-piperidin-1-yl-pyrrolidin-1-yl)-methanone (Compound 340)

Using standard HATU coupling conditions, 3-chloro-6-furan-3-yl-8-trifluoromethyl-imidazo[1,2-a]pyridine-2-carboxylic acid, and 1-pyrrolidin-3-yl-piperidine gave (3-chloro-6-furan-3-yl-8-trifluoromethyl-imidazo[1,2-a]pyridin-2-yl)-(3-piperidin-1-yl-pyrrolidin-1-yl)-methanone (compound 340). ¹H NMR (d₆-DMSO): δ 1.68 (m, 2H), 1.83 (m, 2H), 2.16 (m, 1H), 2.39 (m, 1H), 2.98 (m, 2H), 3.73 (m, 2H), 3.82 (m, 2H), 3.96 (m, 2H), 4.12 (m, 2H), 7.30 (m, 1H), 7.82 (m, 1H), 8.20 (brs, 1H), 8.54 (s, 1H), 8.80 (s, 0.5H), 8.82 (s, 0.5H), 9.68 (brs, 1H); MS (ESI) m/z=467.0 (MH⁺).

### Example 241

### (3-Chloro-6-furan-3-yl-8-trifluoromethyl-imidazo[1,2-a]pyridin-2-yl)-[3-(2-chloro-phenyl)-pyrrolidin-1-yl]-methanone (Compound 341)

Using standard HATU coupling conditions, 3-chloro-6-furan-3-yl-8-trifluoromethyl-imidazo[1,2-a]pyridine-2-carboxylic acid, and 3-(2-chlorophenyl)pyrrolidine gave (3-chloro-6-furan-3-yl-8-trifluoromethyl-imidazo[1,2-a]pyridin-2-yl)-[3-(2-chloro-phenyl)-pyrrolidin-1-yl]-methanone (compound 341). ¹H NMR (d₆-DMSO): δ 2.12 (m, 1H), 2.27 (m, 1H), 3.51 (m, 0.5H), 3.63 (m, 0.5H), 3.77 (m, 2H), 3.90 (m, 0.5H), 4.03 (m, 1H), 4.32 (m, 0.5H), 7.29 (m, 3H), 7.43 (m, 2H), 7.81 (m, 1H), 8.15 (s, 0.5H), 8.18 (s, 0.5H), 8.52 (s, 0.5H), 8.54 (s, 0.5H), 8.78 (s, 0.5H), 8.80 (s, 0.5H); MS (ESI) m/z=493.9 (MH⁺).

### Example 242

### 3-Chloro-6-furan-2-yl-8-trifluoromethyl-imidazo[1,2-a]pyridine-2-carboxylic Acid(tetrahydro-pyran-2-ylmethyl)-amide (Compound 342)

Using standard HATU coupling conditions, 3-chloro-6-furan-2-yl-8-trifluoromethyl-imidazo[1,2-a]pyridine-2-carboxylic acid, and C-(tetrahydro-pyran-2-yl)-methylamine gave 3-chloro-6-furan-2-yl-8-trifluoromethyl-imidazo[1,2-a]pyridine-2-carboxylic acid (tetrahydro-pyran-2-ylmethyl)-amide (compound 342). MS (ESI) m/z=428 (MH⁺).

### Example 243

### 3-Chloro-6-furan-2-yl-8-trifluoromethyl-imidazo[1,2-a]pyridine-2-carboxylic Acid Tetrahydro-pyran-4-ylmethyl)-amide (Compound 343)

Using standard HATU coupling conditions, 3-chloro-6-furan-2-yl-8-trifluoromethyl-imidazo[1,2-a]pyridine-2-carboxylic acid, and C-(tetrahydro-pyran-4-yl)-methylamine gave 3-chloro-6-furan-2-yl-8-trifluoromethyl-imidazo[1,2-a]pyridine-2-carboxylic acid tetrahydro-pyran-4-ylmethyl)-amide (compound 343). MS (ESI) m/z=428.1 (MH⁺).

### Example 244

### 3-Chloro-6-furan-2-yl-8-trifluoromethyl-imidazo[1,2-a]pyridine-2-carboxylic Acid (3-dimethylamino-tetrahydro-thiophen-3-ylmethyl)-amide (Compound 344)

Using standard HATU coupling conditions, 3-chloro-6-furan-2-yl-8-trifluoromethyl-imidazo[1,2-a]pyridine-2-carboxylic acid, and (3-aminomethyl-tetrahydro-thiophen-3-yl)-dimethyl-amine gave 3-chloro-6-furan-2-yl-8-trifluoromethyl-imidazo[1,2-a]pyridine-2-carboxylic acid (3-dimethylamino-tetrahydro-thiophen-3-ylmethyl)-amide (compound 344). MS (ESI) m/z=473.1 (MH⁺).

### Example 245

### (3-Chloro-6-furan-2-yl-8-trifluoromethyl-imidazo[1,2-a]pyridin-2-yl)-pyrrolidin-1-yl-methanone (Compound 345)

Using standard HATU coupling conditions, 3-chloro-6-furan-2-yl-8-trifluoromethyl-imidazo[1,2-a]pyridine-2-carboxylic acid, and pyrrolidine gave (3-chloro-6-furan-2-yl-8-trifluoromethyl-imidazo[1,2-a]pyridin-2-yl)-pyrrolidin-1-yl-methanone (compound 345). MS (ESI) m/z=384 (MH⁺).

### Example 246

### 1-(6-Furan-3-yl-8-trifluoromethyl-imidaz [1,2-a]pyridine-2-carbonyl)-piperidine-4-carboxylic acid ethyl ester (Compound 346)

Using standard HATU coupling conditions, 6-furan-3-yl-8-trifluoromethyl-imidazo[1,2-a]pyridine-2-carboxylic acid, and piperidine-4-carboxylic acid ethyl ester gave 1-(6-furan-3-yl-8-trifluoromethyl-imidazo[1,2-a]pyridine-2-carbonyl)-piperidine-4-carboxylic acid ethyl ester (compound 346). MS (ESI) m/z=436.1 (MH⁺).

### Example 247

### 7-Chloro-5-(1H-pyrazol-4-yl)-1H-indole-2-carboxylic Acid (thiophen-2-ylmethyl)-amide (Compound 347)

A mixture of 5-bromo-7-chloro-1H-indole-2-carboxylic acid (thiophen-2-ylmethyl)-amide (50 mg, 0.13 mmol), 3-pyrazole boronic acid (30 mg, 0.26 mmol), and tetrakis(triphenylphosphine)palladium(0) (5 mol %) was heated in 3M K₃PO₄ (0.45 mL) and 1,4-dioxane (3 mL) at 130° C. for 20 min under microwave conditions. The precipitate was filtered, diluted with EtOAc (25 mL) and washed with saturated aqueous NaHCO₃ (15 mL), then brine (15 mL). The organic extracts were filtered through a small pad of silica gel and the solvent was removed under reduced pressure. The product was purified by preparative TLC [MeOH/CH₂Cl₂ (6:94 v/v)] followed by reverse phase HPLC (30-80% CH₃CN in water (0.1% TFA)) to provide 7-chloro-5-(1H-pyrazol-4-yl)-1H-indole-2-carboxylic acid (thiophen-2-ylmethyl)-amide (compound 347) (5.0 mg, 20%) as a white powder. ¹H NMR (d₆-DMSO, 300 MHz) δ 4.68 (d, 1H, J=5.4 Hz), 6.96 (m, 1H), 7.06 (s, 1H), 7.15 (s, 1H), 7.41 (m, 1H), 7.57 (s, 1H), 7.8 (s, 1H), 8.06 (s, 1H), 9.15 (s, 1H), 11.65 (s, 1H); MS (ESI) m/z=357 (MH⁺).

### Example 248

### 7-Chloro-5-furan-3-yl-1H-indole-2-carboxylic Acid (thiophen-2-ylmethyl)-amide (Compound 348)

7-Chloro-5-furan-3-yl-1H-indole-2-carboxylic acid (thiophen-2-ylmethyl)-amide (compound 348) was prepared using Suzuki coupling as in the preparation of 7-chloro-5-(1H-pyrazol-4-yl)-1H-indole-2-carboxylic acid (thiophen-2-ylmethyl)-amide (compound 347). ¹H NMR (d₆-DMSO, 300 MHz) δ 4.61 (d, 2H, J=3.3 Hz), 6.91 (dd, 1H, J=3.6, 5.1 Hz), 7.0 (s, 1H), 7.12 (m, 1H), 7.33 (m, 1H), 7.53 (s, 1H), 7.64 (s, 1H), 7.77 (s, 1H), 8.12 (s, 1H), 9.12 (m, 1H), 11.64 (s, 1H); MS (ESI) m/z=357 (MH⁺).

### Example 249

### 5-Furan-3-yl-7-trifluoromethyl-1H-benzoimidazole-2-carboxylic Acid (Compound 349)

### Step 1: N-(4-Bromo-2-trifluoromethyl-phenyl)-oxalamic Acid Ethyl Ester

To a solution of 4-bromo-2-trifluoromethyl-phenylamine (500 mg, 0.2 mmol) in THF (1 mL) was added triethylamine (0.56 mL, 4.0 mmol) in THF (1 mL). The mixture was stirred for 15 min and chloro-oxo-acetic acid ethyl ester (400 mg, 0.28 mmol) was added. After 2 hours, the mixture was partitioned between ethyl acetate and water. The organic layer was washed (water, brine), dried to afford the crude product which was purified by flash chromatography [EtOAc/n-hex (30:70 v/v)] to give N-(4-bromo-2-trifluoromethyl-phenyl)-oxalamic acid ethyl ester (650 mg, 92%). MS (ESI) m/z=341 (MH⁺).

### Step 2: N-(4-Bromo-2-nitro-6-trifluoromethyl-phenyl)-oxalamic Acid Ethyl Ester

To a solution of N-(4-bromo-2-trifluoromethyl-phenyl)-oxalamic acid ethyl ester (200 mg, 0.5 mmol) in conc. H₂SO₄ (1 mL) at 0° C. was added cone nitric acid (0.2 mol). The mixture was allowed to stir at 0-10° C. for 2 hours. The mixture was poured on to ice-water to give a precipitate which was filtered, washed with water (2×10 mL) to provide N-(4-bromo-2-nitro-6-trifluoromethyl-phenyl)-oxalamic acid ethyl ester (180 mg, 80%) as a yellow solid. MS (ESI) m/z=386 (MH⁺).

### Step 3: N-(2-Amino-4-bromo-6-trifluoromethyl-phenyl)-oxalamic Acid Ethyl Ester

To a stirred solution of the N-(4-bromo-2-nitro-6-trifluoromethyl-phenyl)-oxalamic acid ethyl ester (2.0 g, 5 mmol) in THF (10 mL) was added a solution of Na₂S2O₄ (8.7 g, 50 mmol) in water (50 mL). After 1 hour, EtOAc was added and the layers were separated. The organic extracts were dried (MgSO₄) and concentrated to provide crude N-(2-amino-4-bromo-6-trifluoromethyl-phenyl)-oxalamic acid ethyl ester (90%) which was used for the next step without further purification. MS (ESI) m/z=355 (MH⁺).

### Step 4: 5-Furan-3-yl-7-trifluoromethyl-1H-benzoimidazole-2-carboxylic Acid (compound 349)

A mixture of N-(2-amino-4-bromo-6-trifluoromethyl-phenyl)-oxalamic acid ethyl ester (50.0 mg, 0.10 mmol), 3-furan boronic acid (31.0 mg, 0.2 mmol), and tetrakis(triphenylphosphine)palladium(0) (5 mol %) was heated in 3M K₃PO₄ (0.5 mL) and 1,4-dioxane (3 mL) under inert atm. at 95° C. for 12 hours. The crude reaction mixture was concentrated and the solid was washed with CH₃CN (5 mL) and water (5 mL) and the crude acid was pure enough to proceed to next step. Sample of the crude material was purified by reverse phase HPLC [30-80% CH₃CN in water (0.1% TFA)] to provide 5-furan-3-yl-7-trifluoromethyl-1H-benzoimidazole-2-carboxylic acid (compound 349) (30 mg, 70%). ¹H NMR (d₆-DMSO, 300 MHz) δ 6.85 (s, 1H), 7.39 (s, 1H), 7.5 (s, 1H), 7.75 (t, 1H, J=1.5 Hz), 8.14 (s, 1H); MS (ESI) m/z=297 (MH⁺).

### Example 250

### 5-Furan-3-yl-7-trifluoromethyl-1H-benzoimidazole-2-carboxylic Acid (thiophen-2-ylmethyl)-amide (Compound 350)

A mixture of 5-furan-3-yl-7-trifluoromethyl-1H-benzoimidazole-2-carboxylic acid (100 mg, 0.33 mmol), thiophen-2-yl-methylamine (76 mg, 0.66 mmol), DIPEA (0.11 mL, 0.66 mmol), HATU (250 mg, 0.66 mmol) was stirred in DMF (1 mL) at 60° C. for 3 hours. The mixture was diluted with EtOAc (25 mL) and washed with saturated aqueous NaHCO₃ (10 mL), then brine (10 mL). The organic phase was dried (MgSO₄), and filtered through a small pad of silica gel. Concentration of the solvent gave the product which was further purified by preparative TLC using 10% MeOH/DCM as an eluent to provide 5-furan-3-yl-7-trifluoromethyl-1H-benzoimidazole-2-carboxylic acid (thiophen-2-ylmethyl)-amide (compound 350) (66 mg, 50%); ¹H NMR (d₆-DMSO, 300 MHz) δ 4.68 (d, 1H, J=6.3 Hz), 6.48 (s, 1H), 6.85 (m, 2H), 7.01 (s, 1H), 7.26 (m, 1H), 7.43 (s, 1H), 7.61 (s, 1H), 7.70 (t, 1H, J=1.5 Hz), 8.16 (s, 1H), 8.49 (t, 1H, J=6.3 Hz); MS (ESI) m/z=392 (MH⁺).

### Example 251

### [3-(4-Fluo ro-phenyl)-pyrrolidin-1-yl]-(6-furan-3-yl-4-trifluoromethyl-1H-benzoimidazol-2-yl)-methanone (Compound 351)

[3-(4-Fluoro-phenyl)-pyrrolidin-1-yl]-(6-furan-3-yl-4-trifluoromethyl-1H-benzoimidazol-2-yl)-methanone (compound 351) was prepared using similar procedure as for 5-furan-3-yl-7-trifluoromethyl-1H-benzoimidazole-2-carboxylic acid (thiophen-2-ylmethyl)-amide (compound 350). ¹H NMR (d₆-DMSO, 300 MHz) δ 2.00 (m, 1H), 2.20 (m, 1H), 3.38 (m, 1.5H), 3.59 (m, 0.5H), 3.83 (m, 1H), 4.00 (m, 1H), 4.40 (s, 0.5H), 4.65 (m, 0.5H), 6.81 (s, 1H), 7.09 (t, 2H, J=8.7 Hz), 7.31 (m, 3H), 7.54 (s, 1H), 7.70 (dd, 1H, J=1.5, 1.8 Hz), 8.13 (s, 1H), 12.09 (s, 1H); MS (ESI) m/z=444 (MH⁺).

### Example 252

### (1-Ethyl-6-furan-3-yl-4-trifluoromethyl-1H-benzoimidazol-2-yl)-[3-(4-fluoro-phenyl)-pyrrolidin-1-yl]-methanone (Compound 352) and

### Example 253

### (1-Ethyl-5-furan-3-yl-7-trifluoromethyl-1H-benzoimidazol-2-yl)-[3-(4-fluoro-phenyl)-pyrrolidin-1-yl]-methanone (Compound 353)

To a solution of [3-(4-fluoro-phenyl)-pyrrolidin-1-yl]-(6-furan-3-yl-4-trifluoromethyl-1H-benzoimidazol-2-yl)-methanone (compound 351) (350 mg, 0.78 mmol) in DMF (2 mL) under inert atm. was added NaH (95%, 38 mg, 1.5 mmol). After 10 min, ethyl iodide (0.2 mL, 2.3 mmol) was added to the mixture which was allowed to stir at room temperature for 12 hours. The brown solution was concentrated and redissolved in ethyl acetate and portioned with water. Evaporation of organic layer gave the crude product which was purified by preparative TLC [15% EtOAc/hexane as eluent] to give (1-ethyl-6-furan-3-yl-4-trifluoromethyl-1H-benzoimidazol-2-yl)-[3-(4-fluoro-phenyl)-pyrrolidin-1-yl]-methanone (compound 352) (40 mg, 10.5%) and (1-ethyl-5-furan-3-yl-7-trifluoromethyl-1H-benzoimidazol-2-yl)-[3-(4-fluoro-phenyl)-pyrrolidin-1-yl]-methanone (compound 353) (18 mg, 5%) both as white powders.

Data for (1-ethyl-6-furan-3-yl-4-trifluoromethyl-1H-benzoimidazol-2-yl)-[3-(4-fluoro-phenyl)-pyrrolidin-1-yl]-methanone (compound 352) ¹H NMR (d₆-DMSO, 300 MHz) δ 1.24 (t, 3H, J=6.6), 2.08 (m, 1H), 2.3 (m, 1H), 3.45 (m, 1.5H), 3.66 (m, 0.5H), 3.91 (m, 1H), 4.08 (m, 1H), 4.34 (bq, 2H), 4.71 (m, 0.5H), 4.83 (m, 0.5H), 7.18 (m, 3H), 7.37 (m, 2H), 7.70 (t, 1H, J=1.5 Hz), 7.71 (bs, 2H), 8.40 (s, 1H); MS (ESI) m/z=472 (MH⁺)

Data for (1-ethyl-5-furan-3-yl-7-trifluoromethyl-1H-benzoimidazol-2-yl)-[3-(4-fluoro-phenyl)-pyrrolidin-1-yl]-methanone (compound 353) ¹H NMR (d₆-DMSO, 300 MHz) δ 1.42 (t, 3H, J=6.9), 2.08 (m, 1H), 2.31 (m, 1H), 3.48 (m, 1H), 3.70 (m, 1H), 3.90 (m, 1H), 4.10 (m, 1H), 4.30 (m, 1H), 4.50 (bq, 2H), 7.18 (m, 3H), 7.41 (m, 2H), 7.76 (s, 1H), 7.95 (s, 1H), 8.00 (s, 1H), 8.40 (s, 1H); MS (ESI) m/z=472 (MH⁺).

### Example 254

### [3-Chloro-6-(3-dimethylaminomethyl-phenyl)-8-trifluoromethyl-imidazo[1,2-a]pyridin-2-yl]-[3-(4-fluoro-phenyl)-pyrrolidin-1-yl]-methanone (Compound 354)

Prepared using similar procedure as for compound 253 (Example 153, Step 4).

¹H NMR (d₆-DMSO, 300 MHz) δ 2.08 (m, 1H), 2.3 (m, 1H), 2.49 (s, 6H), 3.45 (m, 1H), 3.49 (s, 2H), 3.68 (m, 1.5H), 3.85 (m, 1H), 4.05 (m, 1H), 4.26 (m, 0.5H), 7.13 (m, 2H), 7.37 (m, 3H), 7.47 (m, 1H), 7.73 (m, 2H, J=1.5 Hz), 8.13 (d, 1H, J=8.1); 8.75 (d, 1H, J=5.4 Hz); MS (ESI) m/z=546 (MH⁺).

### Example 255

### 1-Ethyl-5-furan-3-yl-7-trifluoromethyl-1H-benzoimidazole-2-carboxylic Acid (thiophen-2-ylmethyl)-amide (Compound 355)

### Step 1: (4-Bromo-2-nitro-6-trifluoromethyl-phenylimino)-ethoxy-acetic Acid Ethyl Ester, 1 and N-(4-Bromo-2-nitro-6-trifluoromethyl-phenyl)-N-ethyl-oxalamic Acid Ethyl Ester

To a stirred solution of N-(4-bromo-2-nitro-6-trifluoromethyl-phenyl)-oxalamic acid ethyl ester (500 mg, 1.2 mmol) and ethyl iodide (0.2 mL, 2.4 mmol) in CH₃CN (2 mL) was added 18-Crown-6 (65 mg, 0.24 mmol) and K₂CO₃ (330 mg, 2.4 mmol). The solution was then stirred at 60° C. for 12 hours. The light brown solution was filtered, reduced in volume and redissolved in ethyl acetate. Flash chromatography [EtOAc/n-hex (15:85 v/v)] of the crude material yielded (4-bromo-2-nitro-6-trifluoromethyl-phenylimino)-ethoxy-acetic acid ethyl ester (29 mg, 5%) and N-(4-bromo-2-nitro-6-trifluoromethyl-phenyl)-N-ethyl-oxalamic acid ethyl ester (430 mg, 81%) as white powder. MS (ESI) m/z=414 (MH⁺).

### Step 2: N-(2-Amino-4-bromo-6-trifluoromethyl-phenyl)-N-ethyl-oxalamic Acid Ethyl Ester

To a stirred solution of N-(4-Bromo-2-nitro-6-trifluoromethyl-phenyl)-N-ethyl-oxalamic acid ethyl ester (100 mg, 0.25 mmol) in THF (1 mL) was added a solution of Na₂S₂O₄ (420 mg, 2.5 mmol) in water (2 mL). After 1 h, ethyl acetate was added and the layers were separated. The extracts were dried (MgSO₄) and evaporated to provide N-(2-amino-4-bromo-6-trifluoromethyl-phenyl)-N-ethyl-oxalamic acid ethyl ester (85 mg, 92%). MS (ESI) m/z=383 (MH⁺).

### Step 3: 1-Ethyl-5-furan-3-yl-7-trifluoromethyl-1H-benzoimidazole-2-carboxylic Acid

Ethyl-5-furan-3-yl-7-trifluoromethyl-1H-benzoimidazole-2-carboxylic acid was prepared using a similar procedure as for 5-furan-3-yl-7-trifluoromethyl-1H-benzoimidazole-2-carboxylic acid (compound 349). MS (ESI) m/z=297 (MH⁺).

### Step 4: 1-Ethyl-5-furan-3-yl-7-trifluoromethyl-1H-benzoimidazole-2-carboxylic Acid(thiophen-2-ylmethyl)-amide (compound 355)

Ethyl-5-furan-3-yl-7-trifluoromethyl-1H-benzoimidazole-2-carboxylic acid (thiophen-2-ylmethyl)-amide (compound 355) was prepared using similar method as for 5-furan-3-yl-7-trifluoromethyl-1H-benzoimidazole-2-carboxylic acid (thiophen-2-ylmethyl)-amide (compound 350). ¹H NMR (d₆-DMSO, 300 MHz) δ 1.34 (t, 3H, J=6.9), 4.66 (d, 2H, J=6.3 Hz), 4.74 (q, 2H, J=7.2 Hz), 6.96 (dd, 1H, J=3.3, 5.1 Hz), 7.05 (m, 1H), 7.15 (m, 1H), 7.40 (m, 1H), 7.78 (t, 1H, J=1.8 Hz), 8.0 (s, 1H), 8.25 (s, 1H), 8.39 (s, 1H), 9.67 (t, 1H, J=6.3 Hz); MS (ESI) m/z=420 (MH⁺).

### Example 256

### Thiophene-2-carboxylic acid (3-chloro-6-furan-3-yl-8-trifluoromethyl-imidazo[1,2-a]pyridin-2-yl)-amide (Compound 356)

A solution of (3-chloro-6-furan-2-yl-8-trifluoromethyl-imidazo[1,2-a]pyridin-2-yl)-carbamic acid tert-butyl ester (80 mg, 0.2 mmol) in THF (1 mL) was added to a suspension of sodium hydride (95%, 10 mg, 04 mmol) in THF (5 mL). After 15 min, thiophene carbonyl chloride (60 mg, 0.4 mmol) was added and the mixture was stirred at 60° C. for 12 hours. The mixture was partitioned between ethyl acetate and saturated aqueous NaHCO₃. The organic extracts were dried (MgSO₄) and evaporated to provide the crude product. To the crude product in dioxane was added 4M HCl in dioxane (10 eq) and stirred at room temperature for 48 hours. Concentration of the solvents followed by purification using preparative TLC [4% MeOH/DCM as an eluent] gave thiophene-2-carboxylic acid (3-chloro-6-furan-3-yl-8-trifluoromethyl-imidazo[1,2-a]pyridin-2-yl)-amide (compound 356) (16 mg, 20%). ¹H NMR (d₆-DMSO, 300 MHz) δ 6.62 (dd, 1H, J=1.8, 3.3 Hz), 7.17 (t, 1H, J=4.2 Hz), 7.28 (d, 1H, J=3.3), 7.79 (s, 1H), 7.83 (d, 1H, J=4.5 Hz), 8.04 (d, 1H, J=3.6 Hz), 8.09 (s, 1H), 8.64 (s, 1H); MS (ESI) m/z=412 (MH⁺).

### Example 257

### Thiophene-2-sulfonic Acid (3-chloro-6-furan-3-yl-8-trifluoromethyl-imidazo[1,2-a]pyridin-2-yl)-amide (Compound 357)

Using similar procedure as for the preparation of thiophene-2-carboxylic acid (3-chloro-6-furan-3-yl-8-trifluoromethyl-imidazo[1,2-a]pyridin-2-yl)-amide (compound 356) by replacing thiophene carbonyl chloride with thiophene-2-sulfonyl chloride gave thiophene-2-sulfonic acid (3-chloro-6-furan-3-yl-8-trifluoromethyl-imidazo[1,2-a]pyridin-2-yl)-amide (compound 357). ¹H NMR (d₆-DMSO, 300 MHz) δ 6.59 (dd, 1H, J=1.8, 3.3 Hz), 7.07 (dd, 1H, J=3.9, 4.8 Hz), 7.21 (d, 1H, J=3.3 Hz), 7.60 (dd, 1H, J=1.5, 3.9 Hz), 7.76 (m, 1H), 7.82 (d, 1H, J=3.9 Hz), 8.01 (s, 1H), 8.55 (s, 1H); MS (ESI) m/z=448 (MH⁺).

### Example 258

### 3-Chloro-8-isopropenyl-6-phenyl-imidazo [1,2-a]pyridine-2-carboxylic Acid (thiophen-2-ylmethyl)-amide (Compound 358)

Prepared using similar procedure as for compound 349 (Example 249, Step 4).

¹H NMR (d₆-DMSO, 300 MHz) δ 2.49 (s, 3H), 4.64 (d, 2H, J=6.0 Hz), 5.58 (s, 1H), 6.71 (s, 1H), 6.95 (dd, 1H, J=3.3, 5.1 Hz), 7.02 (m, 1H), 7.36 (dd, 1H, J=1.2, 5.1 Hz), 7.45 (m, 4H), 7.68 (s, 1H), 7.81 (s, 1H), 7.83 (s, 1H), 8.46 (s, 1H), 9.01 (t, 1H, J=6.0 Hz); MS (ESI) m/z=408 (MH⁺).

### Example 259

### 3-Chloro-6-phenyl-8-styryl-imidazo[1,2-a]pyridine-2-carboxylic Acid (thiophen-2-ylmethyl)-amide (Compound 359)

Prepared using similar procedure as for compound 349 (Example 249, Step 4), (75%).

¹H NMR (d₆-DMSO, 300 MHz) δ 4.70 (d, 2H, J=6.3), 6.97 (dd, 1H, J=3.3, 4.8 Hz), 7.06 (s, 1H), 7.44 (m, 5H), 7.54 (m, 2H), 7.63 (d, 1H, J=16.5 Hz), 7.77 (d, 2H, J=7.8 Hz), 7.85 (d, 2H, J=7.5 Hz), 8.04 (s, 1H), 8.41 (d, 1H, J=16.5 Hz), 8.46 (s, 1H), 9.21 (t, 1H, J=6.0 Hz); MS (ESI) m/z=471 (MH⁺).

### Example 260

### 3-Chloro-6-furan-3-yl-8-trifluoromethyl-imidazo[1,2-a]pyridine-2-carboxylic Acid (thiazol-5-ylmethyl)-amide (Compound 360)

Prepared using similar procedure as for compound 157 (Example 57).

¹H NMR (d₆-DMSO, 300 MHz) δ 4.57 (d, 2H, J=5.7 Hz), 7.25 (s, 1H), 7.38 (s, 1H), 7.76 (s, 1H), 8.15 (s, 1H), 8.49 (s, 1H), 8.66 (t, 1H, J=6.3 Hz), 8.74 (s, 1H), 8.98 (s, 1H); MS (ESI) m/z=427 (MH⁺).

### Example 261

### 3-Bromo-6-phenyl-imidazo[1,2-a]pyridine-2,8-dicarboxylic Acid 8-amide 2-[(thiophen-2-ylmethyl)-amide](Compound 361)

Bromination of 2-amino-nicotinonitrile with NBS followed by treatment with methyl bromopyruvate gave 6-bromo-8-cyano-imidazo[1,2-a]pyridine-2-carboxylic acid ethyl ester. 8-Cyano-6-phenyl-imidazo[1,2-a]pyridine-2-carboxylic acid ethyl ester was obtained from Suzuki reaction of the above bromide with phenylboronic acid. To a stirred solution of 8-cyano-6-phenyl-imidazo[1,2-a]pyridine-2-carboxylic acid ethyl ester (0.038 g, 0.13 mmol) in THF (1 mL) and ethanol (1 mL) was added NaOH (5% aq, 0.5 mL). After 4 hours, the organics were evaporated and the mixture was acidified to pH 4. The mixture was partitioned between EtOAc and water, followed by extraction and drying of the organic layer to afford 8-carbamoyl-6-phenyl-imidazo[1,2-a]pyridine-2-carboxylic acid (0.015 g) as a solid. MS (ESI) m/z=282.1 (M+H⁺). A solution of the acid and NBS (0.009 g, 0.05 mmol) was stirred in DMF (0.5 mL) for 1 hour. Concentration of the solvent followed by aqueous workup afforded 3-bromo-8-carbamoyl-6-phenyl-imidazo[1,2-a]pyridine-2-carboxylic acid (0.017 g, 95%). MS (ESI) m/z=360.0 (M⁺+1). This acid was coupled to thioiphen-2-methylamine under standard HATU coupling conditions to give 3-bromo-6-phenyl-imidazo[1,2-a]pyridine-2,8-dicarboxylic acid 8-amide 2-[(thiophen-2-ylmethyl)-amide](compound 361). MS (ESI) m/z=455.0 (M⁺), 478 (MNa⁺).

### Example 262

### 3-Bromo-8-cyano-6-phenyl-imidazo[1,2-a]pyridine-2-carboxylic Acid (thiophen-2-ylmethyl)amide (Compound 362)

To a stirred solution of 8-cyano-6-phenyl-imidazo[1,2-a]pyridine-2-carboxylic acid ethyl ester (0.1 g, 0.34 mmol) in EtOH (1 mL) and THF (2 mL) was added aqueous NaOH (5%, 0.05 mL) solution. After 30 min, additional THF (6 mL) and aqueous NaOH (5%, 0.05 mL) solution were added and the reaction was monitored until completion (1 hour). The organics were removed and the aqueous layer was acidified to pH 4 to give a solid. The solid was filtered and dried under vacuum to afford 8-cyano-6-phenyl-imidazo[1,2-a]pyridine-2-carboxylic acid (0.052 g, 58%). MS (ESI) m/z=264.1 (M+H⁺) Bromination of 8-cyano-6-phenyl-imidazo[1,2-a]pyridine-2-carboxylic acid followed by amide bond coupling with thioiphen-2-methylamine (as described for compound 361) gave 3-bromo-8-cyano-6-phenyl-imidazo[1,2-a]pyridine-2-carboxylic acid (thiophen-2-ylmethyl)amide (compound 362). ¹H NMR (d₆-DMSO, 300 MHz) δ 4.62 (d, 2H, J=6 Hz), 6.96 (m, 1H), 7.02 (brs, 1H), 7.36 (d, 1H, J=3.9 Hz), 7.50 (m, 3H), 7.82 (d, 2H, J=8.4 Hz), 8.61 (s, 1H), 8.74 (s, 1H), 9.09 (t, 1H, J=5.4 Hz); MS (ESI) m/z=437.0 (M⁺).

### Example 263

### N-(3-Chloro-6-furan-2-yl-8-trifluoromethyl-imidazo[1,2-a]pyridin-2-yl)-C-phenyl-methanesulfonamide (Compound 363)

(6-Furan-2-yl-8-trifluoromethyl-imidazo[1,2-a]pyridin-2-yl)-carbamic acid tert-butyl ester (0.13 g, 0.32 mmol) in THF (1 mL) was added to a suspension of NaH (60%, 0.089 g, 2.2 mmol) in THF (2 mL). After 30 min, phenyl-methanesulfonyl chloride (0.43 g, 2.2 mmol) was added dropwise and stirred for 2 hours. After aqueous workup, and silica gel chromatography, the compound obtained was treated with HCl (4M in dioxane, 3 mL) in anhydrous MeOH (3 mL). After 24 hours, the solvent was concentrated under vacuum. The product was precipitated upon addition of acetonitrile (1 mL) and HCl (1N, 2 mL). The precipitate was filtered and dried under high vacuum to give N-(3-chloro-6-furan-2-yl-8-trifluoromethyl-imidazo[1,2-a]pyridin-2-yl)-C-phenyl-methanesulfonamide (compound 363) as a solid (0.033 g, 23%). ¹H NMR (d₆-DMSO, 300 MHz) δ 4.79 (s, 2H), 6.68 (m, 1H), 7.35 (m, 3H), 7.47 (m, 2H), 7.85 (d, 1H, J=2.1 Hz), 8.18 (s, 1H), 8.65 (s, 1H), 10.42 (s, 1H); MS (ESI) m/z=456.0 (MH⁺).

### Example 264

### 6-(3-Fluoro-phenyl)-3-morpholin-4-ylmethyl-8-trifluoromethyl-imidazo[1,2-a]pyridine-2-carboxylic Acid (thiophen-2-ylmethyl)-amide (Compound 364)

A mixture of 6-(3-fluorophenyl)-8-trifluoromethyl-imidazo[1,2-a]pyridine-2-carboxylic acid (0.1 g, 0.32 mmol), paraformaldehyde (0.03 g) and morpholine (0.08 g, 0.95 mmol) in acetic acid (2 mL) was heated at 120° C. for 15 min under microwave conditions. Trituation of the crude solid with water (100 mL) gave the desired product which upon filtration and drying gave 6-(3-fluoro-phenyl)-3-morpholin-4-ylmethyl-8-trifluoromethyl-imidazo[1,2-a]pyridine-2-carboxylic acid. This acid was coupled to thioiphen-2-methylamine under standard HATU coupling conditions to give 6-(3-fluoro-phenyl)-3-morpholin-4-ylmethyl-8-trifluoromethyl-imidazo[1,2-a]pyridine-2-carboxylic acid (thiophen-2-ylmethyl)-amide (compound 364). ¹H NMR (d₆-DMSO, 300 MHz) δ 3.45 (m under residual water peak), 3.86 (m, 4H), 4.68 (d, 2H, J=6.0 Hz), 5.19 (brs, 2H), 6.94 (m, 1H), 7.04 (d, 1H, J=2.4 Hz), 7.29 (dt, 1H, J=2.4, 8.7 Hz), 7.36 (m, 1H), 7.56 (m, 1H), 7.85 (d, 1H, J=7.8 Hz), 7.95 (brd, 1H), 8.26 (s, 1H), 9.10 (br t, 1H), 9.39 (s, 1H), 11.41 (brs, 1H); MS (ESI) m/z=519.1 (MH⁺).

### Example 265

### 3-Dimethylaminomethyl-6-(3-fluoro-phenyl)-8-trifluoromethyl-imidazO[1,2-a]pyridine-2-carboxylic Acid (thiophen-2-ylmethyl)-amide (Compound 365)

3-Dimethylaminomethyl-6-(3-fluoro-phenyl)-8-trifluoromethyl-imidazo[1,2-a]pyridine-2-carboxylic Acid (thiophen-2-ylmethyl)-amide (compound 365) was prepared similar to compound (compound 364) with the use of dimethylamine instead of morpholine. ¹H NMR (d₆-DMSO, 300 MHz) δ 2.81 (s, 3H), 2.88 (s, 3H), 4.68 (d, 2H, J=6.3 Hz), 5.13 (d, 2H, J=5.1 Hz), 6.95 (m, 1H), 7.04 (m, 1H), 7.27-7.38 (m, 2H), 7.58 (m, 1H), 7.80 (d, 1H, J=8.7 Hz), 8.27 (s, 1H), 7.88 (m, 1H), 9.14 (t, 1H, J=6.0 Hz), 9.34 (s, 1H), 10.41 (brs, 1H); MS (ESI) m/z=477.1 (MH⁺).

### Example 266

### 6-(3-Fluoro-phenyl)-3-pyrrolidin-1-ylmethyl-8-trifluoromethyl-imidazo[1,2-a]pyridine-2-carboxylic Acid (thiophen-2-ylmethyl)-amide (Compound 366)

6-(3-Fluoro-phenyl)-3-pyrrolidin-1-ylmethyl-8-trifluoromethyl-imidazo[1,2-a]pyridine-2-carboxylic acid (thiophen-2-ylmethyl)-amide (compound 366) was prepared similar to compound (compound 364) with the use of pyrrolidine instead of morpholine. ¹H NMR (d₆-DMSO, 300 MHz) δ 1.88 (m, 2H), 2.07 (m, 2H), 3.50-3.35 (m under residual water peak), 4.68 (d, 2H, J=6 Hz), 5.22 (d, 2H, J=5.4 Hz), 6.95 (m, 1H), 7.03 (m, 1H), 7.30 (dt, 1H, J=2.4, 8.4 Hz), 7.36 (dd, 1H, J=5.1, 1.5 Hz), 7.56 (m, 1H), 7.83 (d, 1H, J=8.7 Hz), 7.91 (m, 1H), 8.27 (s, 1H), 9.11 (t, 1H, J=6 Hz), 9.36 (s, 1H), 10.81 (brs, 1H); MS (ESI) m/z=503.1 (MH⁺).

### Example 267

### 3-Bromo-6-(3-fluoro-phenyl)-8-trifluoromethyl-imidazo[1,2-a]pyridine-2-carboxylic Acid (thiophen-2-ylmethyl)-amid (Compound 367)

A solution of 6-(3-fluorophenyl)-8-trifluoromethyl-imidazo[1,2-a]pyridine-2-carboxylic acid (0.16 g, 0.5 mmol) and NBS (0.09 g, 0.5 mmol) was stirred in DMF (1.5 mL) for 3 hours. The mixture was added dropwise to give a precipitate which was filtered and dried under high vacuum to 3-bromo-6-(3-fluoro-phenyl)-8-trifluoromethyl-imidazo[1,2-a]pyridine-2-carboxylic acid. This acid was coupled to thioiphen-2-methylamine under standard HBTU coupling conditions to give 3-bromo-6-(3-fluoro-phenyl)-8-trifluoromethyl-imidazo[1,2-a]pyridine-2-carboxylic acid (thiophen-2-ylmethyl)-amide (compound 367). ¹H NMR (d₆-DMSO, 300 MHz) δ 4.64 (d, 2H, J=6.3 Hz), 6.95 (m, 1H), 7.02 (brs, 1H), 7.31 (dt, 1H, J=3, 9 Hz), 7.36 (d, 1H, J=5.1 Hz), 7.56 (m, 1H), 7.69 (d, 1H, J=7.8 Hz), 7.78 (brd, 1H), 8.21 (s, 1H), 8.78 (s, 1H), 8.88 (t, 1H, J=6.3 Hz); MS (ESI) m/z=499.7 (MH⁺).

### Example 268

### [3-Bromo-6-(3-fluoro-phenyl)-8-trifluoromethyl-imidazo[1,2-a]pyridin-2-yl]-(3-phenyl-pyrrolidin-1-yl)-methanone (Compound 368)

[3-Bromo-6-(3-fluoro-phenyl)-8-trifluoromethyl-imidazo[1,2-a]pyridin-2-yl]-(3-phenyl-pyrrolidin-1-yl)-methanone (compound 368) was prepared similar to the preparation of compound (compound 367). ¹H NMR (d₆-DMSO, 300 MHz) δ 2.06 (m, 1H), 2.31 (m, 1H), 3.4-4.4 (br m under residual water peak), 7.29 (m, 6H), 7.56 (m, 1H), 7.69 (br t, 1H), 7.78 (m, 1H), 8.17 (s, 0.5H), 8.19 (s, 0.5H), 8.78 (s, 0.5H), 8.77 (s, 0.5H); MS (ESI) m/z=533.7 (MH⁺).

### Example 269

### 3-Bromo-8-chloro-6-phenyl-imidazo[1,2-a]pyridine-2-carboxylic Acid (thiophen-2-ylmethyl)-amide (Compound 369)

3-Chloro-5-phenyl-pyridin-2-ylamine was prepared from chlorination of 5-phenyl-pyridin-2-ylamine by N-chlorosuccinimide. Reaction of 3-chloro-5-phenyl-pyridin-2-ylamine with methyl bromopyruvate afforded 8-chloro-6-phenyl-imidazo[1,2-a]pyridine-2-carboxylic acid methyl ester which was brominated with N-bromosuccinimide followed by subsequent saponification gave 3-bromo-8-chloro-6-phenyl-imidazo[1,2-a]pyridine-2-carboxylic acid. This acid was coupled to thioiphen-2-methylamine under standard HBTU coupling conditions to give 3-bromo-8-chloro-6-phenyl-imidazo[1,2-a]pyridine-2-carboxylic acid (thiophen-2-ylmethyl)-amide (compound 369). ¹H NMR (d₆-DMSO, 300 MHz) δ 4.62 (d, 2H, J=6.3 Hz), 6.94 (m, 1H), 7.02 (m, 1H), 7.36-7.54 (m, 4H), 7.80 (d, 2H, J=7.8 Hz), 8.07 (s, 1H), 8.48 (s, 1H), 9.01 (t, 1H, J=6.3 Hz); MS (ESI) m/z=445.9 (M⁺).

### Example 270

### 3,8-Dichloro-6-phenyl-imidazo[1,2-a]pyridine-2-carboxylic Acid (thiophen-2-ylmethyl)-amide (Compound 370)

Following a similar procedure as for the preparation of 3-bromo-8-chloro-6-phenyl-imidazo[1,2-a]pyridine-2-carboxylic acid (thiophen-2-ylmethyl)-amide (compound 369), 8-chloro-6-phenyl-imidazo[1,2-a]pyridine-2-carboxylic acid methyl ester was chlorinated with N-chlorosuccinimide at the C-3 position which upon subsequent saponification to give 3-chloro-8-chloro-6-phenyl-imidazo[1,2-a]pyridine-2-carboxylic acid. This acid was coupled to thioiphen-2-methylamine under standard HBTU coupling conditions to give 3,8-dichloro-6-phenyl-imidazo[1,2-a]pyridine-2-carboxylic acid (thiophen-2-ylmethyl)-amide (compound 370). ¹H NMR (d₆-DMSO, 300 MHz) δ 4.62 (d, 2H, J=6.3 Hz), 6.95 (m, 1H), 7.02 (m, 1H), 7.36-7.54 (m, 4H), 7.83 (d, 2H, J=7.8 Hz), 8.07 (s, 1H), 8.55 (s, 1H), 9.02 (t, 1H, J=6.3 Hz); MS (ESI) m/z=402.0 (M⁺).

### Example 271

### 8-Bromo-3-chloro-6-phenyl-imidazo[1,2-a]pyridine-2-carboxylic Acid (thiophen-2-ylmethyl)-amide (Compound 371)

8-Bromo-3-chloro-6-phenyl-imidazo[1,2-a]pyridine-2-carboxylic acid (thiophen-2-ylmethyl)-amide (compound 371) was prepared using similar procedure as for the synthesis of compound 369. 3-Bromo-5-phenyl-pyridin-2-ylamine was prepared from bromination of 5-phenyl-pyridin-2-ylamine by N-bromosuccinimide. Reaction of 3-bromo-5-phenyl-pyridin-2-ylamine with methyl bromopyruvate afforded 8-bromo-6-phenyl-imidazo[1,2-a]pyridine-2-carboxylic acid methyl ester which was chlorinated with N-chlorosuccinimide followed by subsequent saponification gave 8-bromo-3-chloro-6-phenyl-imidazo[1,2-a]pyridine-2-carboxylic acid. This acid was coupled to thioiphen-2-methylamine under standard HBTU coupling conditions to give 8-bromo-3-chloro-6-phenyl-imidazo[1,2-a]pyridine-2-carboxylic acid (thiophen-2-ylmethyl)-amide (compound 371). ¹H NMR (d₆-DMSO, 300 MHz) δ 4.62 (d, 2H, J=6.6 Hz), 6.94 (m, 1H), 7.02 (d, 1H, J=3.3 Hz), 7.35-7.52 (m, 4H), 7.80 (d, 2H, J=6.9 Hz), 8.18 (s, 1H), 8.56 (s, 1H), 8.96 (t, 1H, J=6.6 Hz); MS (ESI) m/z=445.9 (M⁺).

### Example 272

### 3-Chloro-6-phenyl-8-(1H-pyrazol-4-yl)-imidazo[1,2-a]pyridine-2-carboxylic Acid (thiophen-2-ylmethyl)-amide (Compound 372)

8-Bromo-3-chloro-6-phenyl-imidazo[1,2-a]pyridine-2-carboxylic acid (thiophen-2-ylmethyl)-amide (compound 371) underwent Suzuki coupling with 4-pyrazoleboronic acid pinaacol ester to give 3-chloro-6-phenyl-8-(1H-pyrazol-4-yl)-imidazo[1,2-a]pyridine-2-carboxylic acid (thiophen-2-ylmethyl)-amide (compound 372). ¹H NMR (d₆-DMSO, 300 MHz) δ 4.67 (d, 2H, J=6.0 Hz), 6.95 (m, 1H), 7.04 (m, 1H), 7.35-7.54 (m, 4H), 7.86 (brd, 2H), 8.12 (d, 1H, J=1.8 Hz), 8.37 (d, 1H, J=1.8 Hz), 8.89 (brs, 2H), 9.34 (t, 1H, J=6.3 Hz); MS (ESI) m/z=434.0 (MH⁺).

### Example 273

### 3-Chloro-8-cyano-6-furan-3-yl-imidazo[1,2-a]pyridine-2-carboxylic Acid (thiophen-2-ylmethyl)-amide (Compound 373)

A solution of 6-bromo-8-cyano-imidazo[1,2-a]pyridine-2-carboxylic acid methyl ester (11.07 g, 39.52 mmol) and NCS (5.3 g, 39.52 mmol) was stirred in DMF (200 mL) for 18 hours. Water (200 mL) and NaHSO₃ (5% aq, 50 mL) were added to give a precipitate. The solids were filtered, washed (water) and dried to afford 6-bromo-3-chloro-8-cyano-imidazo[1,2-a]pyridine-2-carboxylic acid methyl ester (11.2 g, 90%) as a tan solid. 6-Bromo-3-chloro-8-cyano-imidazo[1,2-a]pyridine-2-carboxylic acid methyl ester underwent Suzuki coupling with furan-3-boronic acid to give 3-chloro-8-cyano-6-furan-3-yl-iridazo[1,2-a]pyridine-2-carboxylic acid methyl ester. To a suspension of 3-chloro-8-cyano-6-furan-3-yl-imidazo[1,2-a]pyridine-2-carboxylic acid methyl ester (3.73 g, 12.4 mmol) in THF (100 mL) was added a solution of potassium trimethylsilanolate (1.9 g, 14.9 mmol) in THF (15 mL). After 4 hours, water and EtOAc were added and the aqueous layer was acidified with citric acid (5% aq.). The mixture was filtered, the organic layer was washed and dried to afford 3-chloro-8-cyano-6-furan-3-yl-imidazo[1,2-a]pyridine-2-carboxylic acid (2.3 g, 66%). This acid was coupled to thioiphen-2-methylamine under standard HBTU coupling conditions to give 3-chloro-8-cyano-6-furan-3-yl-imidazo[1,2-a]pyridine-2-carboxylic acid (thiophen-2-ylmethyl)-amide (compound 373). ¹H NMR (d₆-DMSO, 300 MHz) δ 4.61 (d, 2H, J=6.3 Hz), 6.94 (m, 1H), 7.01 (d, 1H, J=3.3 Hz), 7.28 (m, 1H), 7.36 (d, 1H, J=5.1 Hz), 7.82 (m, 1H), 8.46 (s, 1H), 8.59 (s, 1H), 8.84 (s, 1H), 9.08 (t, 1H, J=6.3 Hz); MS (ESI) m/z=383.0 (MH⁺).

### Example 274

### 3-Chloro-6-furan-3-yl-8-[1,2,4]oxadiazol-3-yl-imidazo[1,2-a]pyridine-2-carboxylic Acid(thiophen-2-ylmethyl)-amide (Compound 374)

### Step 1: 3-Chloro-6-furan-3-yl-8-(N-hydroxycarbamimidoyl)-imidazo[1,2-a]pyridine-2-carboxylic Acid (thiophen-2-ylmethyl)-amide

To a suspension of 3-chloro-8-cyano-6-furan-3-yl-imidazo[1,2-a]pyridine-2-carboxylic acid (thiophen-2-ylmethyl)-amide (compound 373, 0.39 g, 1.03 mmol) in EtOH (50 mL) was added hydroxylamine (50% soln., 4 mL), and the mixture was heated to reflux for 30 min. Upon cooling to room temperature, water was added (50 mL) to precipitate the product. The precipitate was filtered and dried under vacuum to afford 3-chloro-6-furan-3-yl-8-(N-hydroxycarbamimidoyl)-imidazo[1,2-a]pyridine-2-carboxylic acid (thiophen-2-ylmethyl)-amide (0.25 g, 58%) as a light yellow solid. ¹H NMR (d₆-DMSO, 300 MHz) δ 4.64 (d, 2H, J=6.0 Hz), 6.55 (brs, 2H), 6.95 (m, 1H), 7.02 (m, 1H), 7.16 (m, 1H), 7.36 (d, 1H, J=5.1 Hz), 7.80 (m, 1H), 8.05 (s, 1H), 8.35 (s, 1H), 8.58 (s, 1H), 9.36 (t, 1H, J=6.3 Hz), 10.02 (s, 1H); MS (ESI) m/z=416.0 (MH⁺).

### Step 2: 3-Chloro-6-furan-3-yl-8-[1,2,4]oxadiazol-3-yl-imidazo[1,2-a]pyridine-2-carboxylic Acid (thiophen-2-ylmethyl)-amide (Compound 374)

To a stirred solution of 3-chloro-6-furan-3-yl-8-(N-hydroxycarbamimidoyl)-imidazo[1,2-a]pyridine-2-carboxylic acid (thiophen-2-ylmethyl)-amide (Example 274 Step 1) (0.069 g, 0.17 mmol) in trimethylorthoformate (2 mL) was added boron trifluoride etherate (2 drops). The mixture was then heated at 70° C. for 16 hours. The crude product was purified by reverse phase HPLC to afford 3-chloro-6-furan-3-yl-8-[1,2,4]oxadiazol-3-yl-imidazo[1,2-a]pyridine-2-carboxylic acid (thiophen-2-ylmethyl)-amide (compound 374) (0.008 g, 11%). ¹H NMR (d₆-DMSO, 300 MHz) δ 4.64 (d, 2H, J=6.0 Hz), 6.94 (m, 1H), 7.02 (brs, 1H), 7.27 (s, 1H), 7.36 (d, 1H, J=5.1 Hz), 7.83 (brs, 1H), 8.35 (s, 1H), 8.50 (s, 1H), 8.76 (m, 2H), 9.86 (s, 1H); MS (ESI) m/z=426.0 (MH⁺).

### Example 275

### 3-Chloro-6-furan-3-yl-8-(5-pentyl-[1,2,4]oxadiazol-3-yl)-imidazo[1,2-a]pyridine-2-carboxylic Acid (thiophen-2-ylmethyl)-amide (Compound 375)

3-Chloro-6-furan-3-yl-8-(N-hydroxycarbamimidoyl)-imidazo[1,2-a]pyridine-2-carboxylic acid (thiophen-2-ylmethyl)-amide (Example 274 Step 1) (0.06 g, 0.14 mmol) was dissolved in DMF (1.5 mL) and hexanoic acid (0.016 g, 0.14 mmol), HBTU (0.06 g, 0.15 mmol) and diisopropylethyl amine (0.04 g, 0.28 mmol) were added. The mixture was stirred at room temperature for 1 hour followed by heating at 70° C. over 3 days. The crude product crashed out from aqueous NaHCO₃ was further purified by column chromatography to afford 3-chloro-6-furan-3-yl-8-(5-pentyl-[1,2,4]oxadiazol-3-yl)-imidazo[1,2-a]pyridine-2-carboxylic acid (thiophen-2-ylmethyl)-amide (compound 375) (0.015 g, 22%). ¹H NMR (d₆-DMSO, 300 MHz) δ 0.88 (t, 3H, J=7.2 Hz), 1.27 (m, 4H), 1.82 (m, 2H), 3.06 (t, 2H, J=6.9 Hz), 4.65 (d, 2H, J=6.3 Hz), 6.94 (m, 1H), 7.02 (m, 1H), 7.26 (s, 1H), 7.36 (d, 1H, J=5.1 Hz), 7.82 (s, 1H), 8.28 (s, 1H), 8.48 (s, 1H), 8.73 (m, 2H); MS (ESI) m/z=496.1 (MH⁺).

### Example 276

### 3-Bromo-6-(1H-pyrazol-4-yl)-8-trifluoromethyl-imidazo[1,2-a]pyridine-2-carboxylic Acid(thiophen-2-ylmethyl)-amide (Compound 376)

6-Bromo-8-trifluoromethyl-imidazo[1,2-a]pyridine-2-carboxylic acid methyl ester underwent Suzuki coupling wih 4-pyrazole boronic acid pinacol ester to give 6-(1H-pyrazol-4-yl)-8-trifluoromethyl-imidazo[1,2-a]pyridine-2-carboxylic acid methyl ester. Saponification of 6-(1H-pyrazol-4-yl)-8-trifluoromethyl-imidazo[1,2-a]pyridine-2-carboxylic acid methyl ester with aqueous NaOH gave 6-(1H-pyrazol-4-yl)-8-trifluoromethyl-imidazo[1,2-a]pyridine-2-carboxylic acid. Bromination of 6-(1H-pyrazol-4-yl)-8-trifluoromethyl-imidazo[1,2-a]pyridine-2-carboxylic acid with N-bromosuccinimide gave 3-bromo-6-(1H-pyrazol-4-yl)-8-trifluoromethyl-imidazo[1,2-a]pyridine-2-carboxylic acid. This acid was coupled to thiophen-2-methylamine under standard HBTU coupling conditions to give 3-bromo-6-(1H-pyrazol-4-yl)-8-trifluoromethyl-imidazo[1,2-a]pyridine-2-carboxylic acid (thiophen-2-ylmethyl)-amide (compound 376). ¹H NMR (d₆-DMSO, 300 MHz) δ 4.63 (d, 2H, J=6.3 Hz), 6.95 (m, 1H), 7.02 (brs, 1H), 7.36 (d, 1H, J=5.1 Hz), 8.20 (brs, 2H), 8.54 (s, 1H), 8.74 (s, 1H), 8.80 (t, 1H, J=6.0 Hz); MS (ESI) m/z=471.7 (MH⁺).

### Example 277

### [3-(2-Fluoro-phenyl)-pyrrolidin-1-yl]-[6-(1H-pyrazol-4-yl)-8-trifluoromethyl-imidazo[1,2-a]pyridin-2-yl]-methanone (Compound 377)

Under standard HBTU coupling conditions, 6-(1H-pyrazol-4-yl)-8-trifluoromethyl-imidazo[1,2-a]pyridine-2-carboxylic acid, and 3-(2-fluorophenyl)pyrrolidine gave [3-(2-fluoro-phenyl)-pyrrolidin-1-yl]-[6-(1H-pyrazol-4-yl)-8-trifluoromethyl-imidazo[1,2-a]pyridin-2-yl]-methanone (compound 377). ¹H NMR (d₆-DMSO, 300 MHz) δ 2.04 (m, 1H), 2.30 (m, 1H), 4.08-3.44 (m, under residual water peak), 4.34 (m, 0.5H), 4.48 (m, 0.5H), 7.18 (m, 2H), 7.29 (m, 1H), 7.40 (brt, 1H), 8.05 (s, 0.5H), 8.07 (s, 0.5H), 8.19 (s, 1H), 8.21 (s, 1H), 8.41 (d, 1H, J=3 Hz), 9.11 (s, 0.5H), 9.11 (s, 0.5H); MS (ESI) m/z=444.1 (MH⁺).

### Example 278

### [3-(3-Fluoro-phenyl)-pyrrolidin-1-yl]-[6-(1H-pyrazol-4-yl)-8-trifluoromethyl-imidazo[1,2-a]pyridin-2-yl]-methanone (Compound 378)

Under standard HBTU coupling conditions, 6-(1H-pyrazol-4-yl)-8-trifluoromethyl-imidazo[1,2-a]pyridine-2-carboxylic acid, and 3-(3-fluorophenyl)pyrrolidine gave [3-(3-fluoro-phenyl)-pyrrolidin-1-yl]-[6-(1H-pyrazol-4-yl)-8-trifluoromethyl-imidazo[1,2-a]pyridin-2-yl]-methanone (compound 378). ¹H NMR (d₆-DMSO, 300 MHz) δ 2.02 (m, 1H), 2.31 (m, 1H), 3.42 (m, under residual water peak), 3.75-4.15 (m, 2H), 4.27 (m, 0.5H), 4.48 (m, 0.5H), 7.06 (t, 1H, J=8.4 Hz), 7.17 (m, 2H), 7.37 (m, 1H), 8.04 (s, 0.5H), 8.06 (s, 0.5H), 8.18 (brs, 2H), 8.40 (d, 1H, J=1.8 Hz), 9.09 (s, 0.5H), 9.11 (s, 0.5H); MS (ESI) m/z=444.7 (MH⁺).

### Example 279

### 3-Chloro-8-cyano-6-(1H-pyrazol-4-yl)-imidazo[1,2-a]pyridine-2-carboxylic Acid (thiophen-2-ylmethyl)-amide (Compound 379)

3-Chloro-8-cyano-6-(1H-pyrazol-4-yl)-imidazo[1,2-a]pyridine-2-carboxylic acid (thiophen-2-ylmethyl)-amide (compound 379) was prepared from 6-bromo-8-cyano-imidazo[1,2-a]pyridine-2-carboxylic acid ethyl ester using similar procedures as to 3-chloro-8-cyano-6-furan-3-yl-imidazo[1,2-a]pyridine-2-carboxylic acid (thiophen-2-ylmethyl)-amide (compound 373). ¹H NMR (d₆-DMSO, 300 MHz) δ 4.61 (d, 2H, J=6.3 Hz), 6.94 (m, 1H), 7.01 (d, 1H, J=2.7 Hz), 7.35 (dd, 1H, J=0.9, 4.8 Hz), 8.34 (brs, 2H), 8.59 (s, 1H), 8.85 (s, 1H), 9.05 (t, 1H, J=6.3 Hz); MS (ESI) m/z=383.7 (MH⁺).

### Example 280

### (3-Chloro-6-furan-2-yl-8-trifluoromethyl-imidazo[1,2-a]pyridin-2-yl)-(2,3-dihydro-indol-1-yl)-methanone (Compound 380)

Using standard HATU coupling conditions, 3-chloro-6-furan-2-yl-8-trifluoromethyl-imidazo[1,2-a]pyridine-2-carboxylic acid, and 2,3-dihydro-1H-indole gave (3-chloro-6-furan-2-yl-8-trifluoromethyl-imidazo[1,2-a]pyridin-2-yl)-(2,3-dihydro-indol-1-yl)-methanone (compound 380). ¹H NMR (d₆-DMSO): δ 3.17 (t, 2H, J=8.4 Hz), 4.44 (t, 2H, J=8.4 Hz), 6.69 (dd, 1H, J=1.8, 3.3 Hz), 7.07 (t, 1H, J=7 Hz), 7.22 (m, 1H), 7.29 (d, 1H, J=7 Hz), 7.39 (d, 1H, J=3.3 Hz), 7.87 (d, 1H, J=1.2 Hz), 8.17 (d, 1H, J=8.1 Hz), 8.25 (s, 1H), 8.72 (s, 1H); MS (ESI) m/z=432 (MH⁺).

### Example 281

### (3-Chloro-6-furan-3-yl-8-trifluoromethyl-imidazo[1,2-a]pyridin-2-yl)-(3-morpholin-4-yl-pyrrolidin-1-yl)-methanone (Compound 381)

Using standard HATU coupling conditions, 3-chloro-6-furan-3-yl-8-trifluoromethyl-imidazo[1,2-a]pyridine-2-carboxylic acid, and 4-pyrrolidin-3-yl-morpholine gave (3-chloro-6-furan-3-yl-8-trifluoromethyl-imidazo[1,2-a]pyridin-2-yl)-(3-morpholin-4-yl-pyrrolidin-1-yl)-methanone (compound 381). ¹H NMR (d₆-DMSO): δ 2.20-2.44 (m, 2H), 3.08-4.30 (m, 13H), 7.31 (s, 1H), 7.82 (t, 1H, J=1.5 Hz), 8.20 (s, 1H), 8.54 (s, 1H), 8.81 (brs, 1H); MS (ESI) m/z=469 (MH⁺).

### Example 282

### 3-Chloro-6-furan-3-yl-N-thiophen-2-ylmethyl-8-trifluoromethyl-imidazo[1,2-a]pyridine-2-carboxamidine (Compound 382)

### Step 1: 3-Chloro-6-furan-3-yl-8-trifluoromethyl-imidazo[1,2-a]pyridine-2-carboxylic Acid Amide

The title compound was prepared from 3-chloro-6-furan-3-yl-8-trifluoromethyl-imidazo[1,2-a]pyridine-2-carboxylic acid and ammonium chloride using standard HATU coupling conditions. MS (ESI) m/z=330.0 (MH⁺).

### Step 2: 3-Chloro-6-furan-3-yl-8-trifluoromethyl-imidazo[1,2a]pyridine-2-carbonitrile

3-Chloro-6-furan-3-yl-8-trifluoromethyl-imidazo[1,2-a]pyridine-2-carboxylic acid amide (0.93 g, 2.8 mmol) was refluxed in POCl₃ (10 mL) for 1 hour. POCl₃ was removed under vacuum and the residue was suspended in EtOAc/water. The solids that remained undissolved were filtered and the filtrate subjected to a normal extractive workup. The organic layer was concentrated and the solids obtained were combined with the previously collected solids (above) to afford the crude product. Trituration of the crude solid with ether (15 mL) afforded the desired product (0.7 g, 79%) as a tan solid. MS (ESI) m/z=312.0 (MH⁺).

### Step 3: 3-Chloro-6-furan-3-yl-N-thiophen-2-ylmethyl-8-trifluoromethyl-imidazo[1,2-a]pyridine-2-carboxamidine (compound 382)

3-Chloro-6-furan-3-yl-8-trifluoromethyl-imidazo[1,2a]pyridine-2-carbonitrile (0.11 g, 0.35 mmol), CuCl (0.038 g, 0.38 mmol) and thiophen-2-yl-methylamine (0.06 g, 0.53 mmol) were suspended in EtOH (2 mL) and the mixture heated at 120° C. for 10 min under microwave conditions. The reaction mixture was poured into 5% aqueous NaOH solution and the mixture sonicated and heated gently. The mixture was then acidified to pH 2 with 1N HCl and filtered. The crude product contained in the filtrate was purified by reverse phase HPLC to afford the title compound (0.026 g, 17%). ¹H NMR (d₆-DMSO, 300 MHz) δ 4.96 (d, 2H, J=5.7 Hz), 7.05 (t, 1H, J=4.8 Hz), 7.26 (m, 1H), 7.35 (s, 1H), 7.54 (d, 1H, J=5.1 Hz), 8.33 (s, 1H), 8.59 (s, 1H), 8.91 (s, 1H), 8.65 (s, 1H), 8.81 (s, 1H), 10.39 (br t, 1H); MS (ESI) m/z=425.0 (MH⁺).

### Example 283

### N-{3-Chloro-2-[3-(3-fluoro-phenyl)-pyrrolidine-1-carbonyl]-6-furan-3-yl-imidazo[1,2-a]pyridin-8-yl}-methanesulfonamide (Compound 383)

### Step 1: 6-Bromo-3-chloro-8-nitro-imidazo[1,2-a]pyridine-2-carboxylic Acid Methyl Ester

5-Bromo-3-nitro-pyridin-2-ylamine was converted to 6-bromo-8-nitro-imidazo[1,2-a]pyridine-2-carboxylic acid methyl ester which was then converted to 6-bromo-3-chloro-8-nitro-imidazo[1,2-a]pyridine-2-carboxylic acid methyl ester [MS (ESI) m/z=301.9 (MH⁺)] using procedures as described previously for the synthesis of 6-bromo-3-chloro-8-trifluoromethyl-imidazo[1,2-a]pyridine-2-carboxylic acid methyl ester. MS (ESI) m/z=335.9 (MH⁺).

### Step 2: 8-Amino-6-bromo-3-chloro-imidazo[1,2-a]pyridine-2-carboxylic Acid Methyl Ester

6-Bromo-3-chloro-8-nitro-imidazo[1,2-a]pyridine-2-carboxylic acid methyl ester (1.12 g, 3.3 mmol) was dissolved in THF (200 mL) and Na₂S₂O₄ (6.8 g) in water (50 mL) was added and the mixture stirred for 2 hours. Aqueous NaOH solution (5%) was added until the mixture reached a pH of 8-9. The mixture was extracted with EtOAc (4×100 mL) to give crude 8-amino-6-bromo-3-chloro-imidazo[1,2-a]pyridine-2-carboxylic acid methyl ester (0.3 g) which was used for the next step without further purification. MS (ESI) m/z=306.0 (MH⁺).

### Step 3: 6-Bromo-3-chloro-8-methanesulfonylamino-imidazo[1,2-a]pyridine-2-carboxylic Acid Methyl Ester

8-Amino-6-bromo-3-chloro-imidazo[1,2-a]pyridine-2-carboxylic acid methyl ester (0.28 g, 0.92 mmol) was dissolved in DCM (2 mL) and methanesulfonyl chloride (0.11 g, 0.92 mol) and triethylamine (0.27 mL, 1.84 mmol) were added and the mixture stirred for 18 hours. Additional methanesulfonyl chloride (0.11 g, 0.92 mmol) and triethylamine (0.27 mL, 1.84 mmol) were added and the mixture stirred an additional 5 hours. The DCM was removed under vacuum and water (25 mL) and EtOAc (50 mL) were added. After an extractive work-up, the organic layer was concentrated and subsequently redissolved in THF (5 mL). Aqueous NaOH solution (0.5%, 1 mL) was added and the mixture stirred for 1 hour. The mixture was acidified to pH 4 with 1N HCl, and crude 6-bromo-3-chloro-8-methanesulfonylamino-imidazo[1,2-a]pyridine-2-carboxylic acid methyl ester (0.215 g) was obtained by extracting with EtOAc and drying. MS (ESI) m/z=383.9 (MH⁺).

### Step 4: N-{3-Chloro-2-[3-(3-fluoro-phenyl)-pyrrolidine-1-carbonyl]-6-furan-3-yl-imidazo[1,2-a]pyridin-8-yl}-methanesulfonamide (compound 383)

6-Bromo-3-chloro-8-methanesulfonylamino-imidazo[1,2-a]pyridine-2-carboxylic acid methyl ester (0.215 g, 0.56 mmol) was subjected to standard Suzuki coupling conditions using 4-pyrazole boronic acid. Under these conditions, a 1:1 mixture (0.16 g) of 3-chloro-8-methanesulfonylamino-6-(1H-pyrazol-4-yl)-imidazo[1,2-a]pyridine-2-carboxylic acid and 6-bromo-3-chloro-8-methanesulfonylamino-imidazo[1,2-a]pyridine-2-carboxylic acid was obtained. This mixture was subjected to HBTU amide coupling conditions with 3-(3-fluoro-phenyl)-pyrrolidine. Purification of the crude reaction mixture afforded the desired product N-{3-chloro-2-[3-(3-fluoro-phenyl)-pyrrolidine-1-carbonyl]-6-furan-3-yl-imidazo[1,2-a]pyridin-8-yl}-methanesulfonamide (0.019 g). ¹H NMR (d₆-DMSO, 300 MHz) δ 10.08 (br s, 1H), 8.44 (s, 1H), 8.25 (s, 2H), 7.51-7.10 (m, 4H), 4.41 (br dd, 0.5H), 4.22-3.43 (m with water peak), 3.26 (s, 1.5H), 3.18 (s, 1.5H), 2.31 (br m, 1H), 2.03 (br m, 1H); MS (ESI) m/z=503.1 (MH⁺).

### Example 284

### N-{3-Chloro-2-[3-(3-fluoro-phenyl)-pyrrolidine-1-carbonyl]-6-furan-3-yl-imidazo[1,2-a]pyridin-8-yl}-acetamide (Compound 384)

### Step 1: 8-Acetylamino-6-bromo-3-chloro-imidazo[1,2-a]pyridine-2-carboxylic Acid Methyl Ester

8-Amino-6-bromo-3-chloro-imidazo[1,2-a]pyridine-2-carboxylic acid methyl ester (0.29 g, 0.98 mmol) was dissolved in pyridine (5 mL), acetic anhydride (1.5 mL) was added and the mixture stirred over 72 hours. The mixture was concentrated, EtOAc/water added and after a normal extractive work up, 8-acetylamino-6-bromo-3-chloro-imidazo[1,2-a]pyridine-2-carboxylic acid methyl ester (0.26 g, 77%) was obtained. MS (ESI) m/z=348.0 (MH⁺).

### Step 2: 8-Acetylamino-3-chloro-6-furan-3-yl-imidazo[1,2-a]pyridine-2-carboxylic Acid Methyl Ester

8-Acetylamino-6-bromo-3-chloro-imidazo[1,2-a]pyridine-2-carboxylic acid methyl ester (0.26 g, 0.75 mmol) was subjected to Suzuki coupling conditions with 3-furanboronic acid to afford 8-acetylamino-3-chloro-6-furan-3-yl-imidazo[1,2-a]pyridine-2-carboxylic acid methyl ester (0.16 g, 64%); MS (ESI) m/z=334.0 (MH⁺), 356 (MNa⁺).

### Step 3: 8-Acetylamino-3-chloro-6-furan-3-yl-imidazo[1,2-a]pyridine-2-carboxylic Acid

8-Acetylamino-3-chloro-6-furan-3-yl-imidazo[1,2-a]pyridine-2-carboxylic acid methyl ester (0.16 g, 0.48 mmol) was dissolved in THF (20 mL) and an aqueous NaOH solution (5%, 2 mL) was added and the mixture stirred for 1 hour. The mixture was concentrated and the mixture acidified to pH 3 with 1N HCl. The crude product crashed out, was filtered, washed with water and dried to afford 8-acetylamino-3-chloro-6-furan-3-yl-imidazo[1,2-a]pyridine-2-carboxylic acid (0.08 g, 52%); MS (ESI) m/z=320 (MH⁺).

### Step 4: N-{3-Chloro-2-[3-(3-fluoro-phenyl)-pyrrolidine-1-carbonyl]-6-furan-3-yl-imidazo[1,2-a]pyridin-8-yl}-acetamide (Compound 384)

Prepared using standard HBTU coupling (0.06 g, 51%). ¹H NMR (d₆-DMSO, 300 MHz) [symbol] 10.05 (br s, 1H), 8.34-8.24 (m, 3H), 7.82 (br s, 1H) 7.41-7.03 (m, 5H), 4.32-3.20 (m under br water peak), 2.31 (m, 1H), 2.28 (s, 1.5H), 2.21 (s, 1.5H), 2.09 (m, 1H); MS (ESI) m/z=467.1 (MH⁺).

### Example 285 and 286

### (3-Chloro-6-furan-3-yl-8-trifluoromethyl-imidazo[1,2-a]pyridin-2-ylmethyl)-carbamic Acid Tert-butyl Ester (Compound 385) and

### (6-Furan-3-yl-8-trifluoromethyl-imidazo[1,2-a]pyridin-2-ylmethyl)-carbamic Acid Tert-butyl Ester (Compound 386)

3-Chloro-6-furan-3-yl-8-trifluoromethyl-imidazo[1,2-a]pyridine-2-carbonitrile (1.95 g, 6.27 mmol) and di-tert-butyl dicarbonate (2.74 g, 12.54 mmol) were dissolved in MeOH (50 mL) and the mixture was cooled to 0° C. Nickel chloride hexahydrate (1.49 g, 6.27 mmol) was added, followed by portion-wise addition of NaBH₄ (1.2 g, 31.35 mmol) over 2 hours. The mixture was allowed to warm to room temperature and MeOH was removed under vacuum. A saturated aqueous solution of NaHCO₃ (20 mL) was added followed by extraction with EtOAc. Solids that remained were filtered off and concentration of the organic layer afforded the crude product (1 g). The solids collected above were suspended in citric acid (5% aq., 20 mL) and extracted with EtOAc to afford additional 0.8 g of crude product. The combined crude products were purified by silica gel chromatography to give (6-furan-3-yl-8-trifluoromethyl-imidazo[1,2-a]pyridin-2-ylmethyl)-carbamic acid tert-butyl ester (0.26 g, 10%) (MS (ESI) m/z=382.1 (MH⁺)) and (3-chloro-6-furan-3-yl-8-trifluoromethyl-imidazo[1,2-a]pyridin-2-ylmethyl)-carbamic acid tert-butyl ester (0.5 g, 19%). MS (ESI) m/z=416.1 (MH⁺).

### Example 287 and 288

### N-(3-Chloro-6-furan-3-yl-8-trifluoromethyl-imidazo[1,2-a]pyridin-2-ylmethyl)-2-thiophen-2-yl-acetamide (Compound 387) and

### N-(6-Furan-3-yl-8-trifluoromethyl-imidazo[1,2-a]pyridin-2-ylmethyl)-2-thiophen-2-yl-acetamide (Compound 388)

### Step 1: C-(3-Chloro-6-furan-3-yl-8-trifluoromethyl-imidazo[1,2-a]pyridin-2-yl)-methylamine and C-(6-furan-3-yl-8-trifluoromethyl-imidazo[1,2-a]pyridin-2-yl)-methylamine

A mixture of (6-furan-3-yl-8-trifluoromethyl-imidazo[1,2-a]pyridin-2-ylmethyl)-carbamic acid tert-butyl ester and (3-chloro-6-furan-3-yl-8-trifluoromethyl-imidazo[1,2-a]pyridin-2-ylmethyl)-carbamic acid tert-butyl ester (0.2 g) was dissolved in anhydrous MeOH (1 mL) and a solution of hydrogen chloride in 1,4-dioxane (4M, 1 mL) was added. The mixture was stirred for 1 hour, then concentrated and dried to afford the crude amino methyl intermediates which were used for the next step without further purification.

### Step 2: N-(3-Chloro-6-furan-3-yl-8-trifluoromethyl-imidazo[1,2-a]pyridin-2-ylmethyl)-2-thiophen-2-yl-acetamide (compound 387) and N-(6-Furan-3-yl-8-trifluoromethyl-imidazo[1,2-a]pyridin-2-ylmethyl)-2-thiophen-2-yl-acetamide (compound 388)

Prepared using standard HBTU coupling of the above mixture of amines with thiophen-2-yl-acetic acid.

Data for N-(3-Chloro-6-furan-3-yl-8-trifluoromethyl-imidazo[1,2-a]pyridin-2ylmethyl)-2-thiophen-2-yl-acetamide: ¹H NMR (d₆-DMSO, 300 MHz) [symbol] 3.69 (s, 2H), 4.45 (d, 2H, J=5.7 Hz), 6.93 (m, 2H), 7.29 (m, 1H), 7.34 (m, 1H), 7.82 (m, 1H), 8.10 (s, 1H), 8.52 (s, 1H), 8.75 (m, 2H); MS (ESI) m/z=440.0 (MH⁺).

Data for N-(6-Furan-3-yl-8-trifluoromethyl-imidazo[1,2-a]pyridin-2-ylmethyl)-2- [symbol] thiophen-2-yl-acetamide: ¹H NMR (d₆-DMSO, 300 MHz) [symbol] 3.73 (s, 2H), 4.44 (d, 2H, J=5.4 Hz), 6.95 (m, 2H), 7.03 (m, 1H), 7.36 (m, 1H), 7.83 (m, 1H), 7.89 (s, 1H), 8.05 (s, 1H), 8.40 (s, 1H), 8.75 (t, 1H, J=5.7 Hz), 9.16 (s, 1H); MS (ESI) m/z=406.1 (MH⁺).

### Example 289 and 290

### N-(3-Chloro-6-furan-3-yl-8-trifluoromethyl-imidazo[1,2-a]pyridin-2-ylmethyl)-2-phenyl-acetamide (Compound 389) and

### N-(6-Furan-3-yl-8-trifluoromethyl-imidazo[1,2-a]pyridin-2-ylmethyl)-2-phenyl-acetamide (Compound 390)

Prepared using similar procedures as in Examples 287 and 288 (compounds 387 and 388).

Data for N-(3-Chloro-6-furan-3-yl-8-trifluoromethyl-imidazo[1,2-a]pyridin-2ylmethyl)-2-phenyl-acetamide: ¹H NMR (d₆-DMSO, 300 MHz) [symbol] 3.47 (s, 2H), 4.44 (d, 2H, J=6.0 Hz), 7.26 (m, 6H), 7.82 (m, 1H), 8.10 (s, 1H), 8.52 (s, 1H), 8.72 (t, 1H, J=5.4 Hz), 8.75 (s, 11-1); MS (ESI) m/z=434.1 (MH⁺).

Data for N-(6-Furan-3-yl-8-trifluoromethyl-imidazo[1,2-a]pyridin-2-ylmethyl)-2-phenyl-acetamide: ¹H NMR (d₆-DMSO, 300 MHz) [symbol] 3.50 (s, 2H), 4.42 (d, 2H, J=5.7 Hz), 7.03 (m, 1H), 7.22-7.30 (m, 5H), 7.82 (m, 1H), 7.88 (s, 1H), 8.05 (s, 1H), 8.40 (s, 1H), 8.72 (t, 1H, J=5.7 Hz), 9.15 (s, 1H); MS (ESI) m/z=400.1 (MH⁺).

### Example 291

### 1-Benzyl-3-(6-furan-3-yl-8-trifluoromethyl-imidazo[1,2-a]pyridin-2-ylmethyl)-urea (Compound 391)

To a mixture of 3-chloro-6-furan-3-yl-8-trifluoromethyl-imidazo[1,2-a]pyridin-2-yl)-methylamine and (6-furan-3-yl-8-trifluoromethyl-imidazo[1,2-a]pyridin-2-yl)-methylamine) (0.044 g) in DMF (1 mL) was added benzyl isocyanate (0.017 mL) and N,N-diisopropylethyl amine (0.08 mL). After stirring for 1 hour, the mixture was concentrated andpurified by reverse phase HPLC to afford the title compound (0.032 g). ¹H NMR (d₆-DMSO, 300 MHz) [symbol] 4.24 (s, 2H), 4.62 (s, 2H), 7.06 (m, 1H), 7.29 (m, 5H), 7.85 (m, 1H), 8.03 (s, 1H), 8.32 (s, 1H), 8.47 (s, 1H), 8.36 (s, 1H); MS (ESI) m/z=415.1 (MH⁺).

### Example 292

### 1-(6-Furan-3-yl-8-trifluoromethyl-imidazo[1,2-a]pyridin-2-ylmethyl)-3-phenyl-urea (Compound 392)

Prepared using similar procedure as in Example 291 (compound 391). ¹H NMR (d₆-DMSO, 300 MHz) [symbol] 4.44 (s, 2H), 6.84 (m, 2H), 7.02 (s, 1H), 7.21 (t, 2H, J=7.5 Hz), 7.41 (d, 2H, J=7.8 Hz), 7.82 (s, 1H), 7.99 (s, 1H), 8.14 (s, 1H), 8.40 (s, 1H), 8.89 (s, 1H), 9.20 (s, 1H); MS (ESI) m/z=401.1 (MH⁺).

### Example 293

### (6-Furan-3-yl-8-trifluoromethyl-imidazo[1,2-a]pyririn-2-ylmethyl-carbamic Acid Benzyl Ester (Compound 393)

### Step 1: C-(6-Furan-3-yl-8-trifluoromethyl-imidazo[1,2-a]pyridine-2-ylmethyl)-carbamic Acid Tert-butyl Ester

6-Bromo-8-trifluoromethyl-imidazo[1,2-a]pyridine-2-carboxylic acid was converted to (6-furan-3-yl-8-trifluoromethyl-imidazo[1,2-a]pyridin-2-ylmethyl)-carbamic acid tert-butyl ester using methods as described for (3-chloro-6-furan-3-yl-8-trifluoromethyl-imidazo[1,2-a]pyridin-2-ylmethyl)-carbamic acid tert-butyl ester as in Example 285 (compound 385)

### Step 2: C-(6-Furan-3-yl-8-trifluoromethyl-imidazo[1,2-a]pyridine-2-yl)-methylamine

C-(6-Furan-3-yl-8-trifluoromethyl-imidazo[1,2-a]pyridine-2-ylmethyl)-carbamic acid tert-butyl ester (103 mg 0.27 mmol) was dissolved in MeOH (2 mL) and a solution of hydrogen chloride in 1,4-dioxane (4N, 0.5 mL) was added. This solution was stirred at room temperature for 2 hours. Concentration of the solvent gave C-(6-furan-3-yl-8-trifluoromethyl-imidazo[1,2-a]pyridine-2-yl)-methylamine (82.3 mg, 96) as an HCl salt. MS (ESI) m/z 282 (MH⁺).

### Step 3: (6-Furan-3-yl-8-trifluoromethyl-imidazo[1,2-a]pyridine-2-ylmethyl)-carbamic Acid Benzyl Ester (compound 393)

C-(6-Furan-3-yl-8-trifluoromethyl-imidazo[1,2-a]pyridine-2-yl)-methylamine (82.3 mg, 0.26 mmol) was suspended in dichloromethane (2.5 mL). To this suspension was added N,N-diisopropylethylamine (0.14 mL, 0.78 mmol) at 0° C. followed by benzyl chloroformate (0.05 mL, 0.39 mmol). The mixture was stirred at 0° C. for 15 minutes and then brought to room temperature and stirred for 15 minutes at room temperature. Reaction mixture was quenched using H₂O and extracted with dichloromethane. The organic phase was separated, dried (MgSO₄), filtered and concentrated to give the crude product. The crude was purified using reverse phase HPLC to give (6-furan-3-yl-8-trifluoromethyl-imidazol-pyridine-2-ylmethyl)-carbamic acid benzyl ester (61 mg, 57%). ¹H NMR (d₆-DMSO, 300 MHz) [symbol] 9.13 (s, 1H), 8.38 (s, 1H), 8.02 (s, 1H), 7.93 (t, 1H, J=6 Hz), 7.87 (s, 1H), 7.80 (br s, 1H), 7.34 (m, 5H), 6.99 (br s, 1H), 5.05 (s, 2H), 4.35 (d, 2H, J=7 Hz); MS (ESI) m/z 416 (MH⁺).

### Example 294

### (6-Furan-3-yl)-8-trifluoromethyl-imidazo[1,2-a]pyridine-2-ylmethyl)-carbamic Acid Phenyl Ester (Compound 394)

Prepared using similar procedure as in Example 293 (compound 393). ¹H NMR (d₆-DMSO, 300 MHz) [symbol] 8.81 (s, 1H), 8.55 (s, 1H), 8.50 (t, 1H), J=6 Hz), 8.23 (s, 1H), 7.85 (d, 1H, J=7 Hz), 7.83 (m, 2H), 7.58 (m, 2H), 7.31 (br s, 1H), 4.82 (d, 2H, J=2 Hz), 3.61 (brs, 1H); MS (ESI) m/z 402 (MH⁺).

### Example 295

### N-(6-Furan-3-yl)-8-trifluoromethyl)-imidazo[1,2-a]pyridine-2-ylmethyl)-benzenesulfonamide (Compound 395)

Prepared following experimental procedure described as in Example 296 (compound 396). ¹H NMR (d₆-DMSO, 300 MHz) [symbol] 9.06 (s, 1H), 8.36 (s, 1H), 8.26 (t, 1H, J=6 Hz), 7.96 (s, 1H), 7.81 (s, 1H), 7.78 (m, 3H), 7.50 (m, 3H), 7.00 (s, 1H), 4.14 (d, 2H, J=6 Hz); MS (ESI) m/z 422 (MH⁺).

### Example 296

### N-(6-Furan-3-yl)-8-trifluoromethyl)-imidazo[1,2-a]pyridine-2-ylmethyl)-C-phenyl-methanesulfonamide (Compound 396)

C-(6-Furan-3-yl-8-trifluoromethyl-imidazo[1,2-a]pyridine-2-yl)-methylamine (50 mg, 0.18 mmol) was suspended in dichloromethane (2.5 mL). To this suspension, was added N,N-diisopropylethylamine (0.09 mL, 0.54 mmol) followed by phenylmethanesulfonyl chloride (44.6 mg, 0.23 mmol). Reaction mixture was stirred at room temperature overnight. It was then quenched using H₂O and extracted with dichloromethane. The organic phase was separated, dried (MgSO₄), filtered and concentrated. The crude product was purified using reverse phase HPLC. ¹H NMR (d₆-DMSO, 300 MHz) [symbol] 9.12 (s, 1H), 8.37 (s, 1H), 7.98 (s, 1H), 7.92 (s, 1H), 7.79 (s, 1H), 7.74 (t, 1H, J=7 Hz), 7.33 (m, 5H), 7.00 (br s, 1H), 4.40 (s, 2H), 4.25 (d, 2H, J=6 Hz); MS (ESI) m/z 436 (MH⁺).

### Example 297

### 1-(4-Fluoro-benzyl)-3-(6-furan-3-yl-8-trifluoromethyl-imidazo[1,2-a]pyridine-2-ylmethyl)-urea (Compound 397)

Prepared following experimental procedure described in Example 292 (compound 392). ¹H NMR (d₆-DMSO, 300 MHz) [symbol] 9.11 (s, 1H), 8.37 (s, 1H), 7.98 (s, 1H), 7.83 (s, 1H), 7.80 (t, 1H, J=2 Hz), 7.26 (m, 2H), 7.10 (m, 2H), 6.99 (br s, 1H), 6.76 (br m, 1H) 4.35 (s, 2H), 4.18 (s, 2H); MS (ESI) m/z 433 (MH⁺).

### Example 298

### 1-(3-Fluoro-benzyl)-3-(6-furan-3-yl-8-trifluoromethyl-imidazo[1,2-a]pyridine-2-ylmethyl)-urea (Compound 398)

Prepared following experimental procedure described in Example 292 (compound 392). ¹H NMR (d₆-DMSO, 300 MHz) [symbol] 9.11 (s, 1H), 8.36 (s, 1H), 7.98 (s, 1H), 7.84 (s, 1H), 7.80 (t, 1H, J=2 Hz), 7.32 (m, 1H), 7.09-6.99 (m, 4H), 6.82 (br m, 1H), 4.36 (s, 2H), 4.23 (s, 2H); MS (ESI) m/z 433 (MH⁺).

### Example 299

### 1-(2-Fluoro-benzyl)-3-(6-furan-3-yl-8-trifluoromethyl-imidazo[1,2-a]pyridine-2-ylmethyl)-urea (Compound 399)

Prepared following experimental procedure described in Example 292 (compound 392). ¹H NMR (d₆-DMSO, 300 MHz) [symbol] 9.13 (s, 1H), 8.37 (s, 1H), 8.02 (s, 1H), 7.86 (s, 1H), 7.80 (t, 1H, J=2 Hz), 7.28 (m, 2H), 7.11 (m, 2H), 7.00 (br s, 1H), 6.58 (br s, 1H), 4.36 (s, 1H), 4.26 (s, 2H); MS (ESI) m/z 433 (MH⁺).

### Example 300

### 1-(3-Fluoro-phenyl)-3-(6-furan-3-yl-8-trifluoromethyl-imidazo[1,2-a]pyridine-2-ylmethyl)-urea (Compound 400)

Prepared following experimental procedure described in Example 292 (compound 392). ¹H NMR (d₆-DMSO, 300 MHz) [symbol] 9.11 (s, 1H), 8.71 (s, 1H), 8.36 (s, 1H), 7.99 (s, 1H), 7.90 (s, 1H), 7.79 (t, 1H, J=2 Hz), 7.39 (m, 2H), 7.02 (m, 3H), 4.42 (br d, 2H, J=3 Hz); MS (ESI) m/z 419 (MH⁺).

### Example 301

### 2-(4-fluorophenyl)-N-{[6-furan-3-yl)-8-trifluoromethyl)imidazo[1,2-a]pyridine-2-yl]methyl}acetamide (Compound 401)

Prepared using exerimental procedures described in Example 293 (compound 393).

¹H NMR (d₆-DMSO, 300 MHz) [symbol] 9.10 (s, 1H), 8.67 (t, 1H, J=6 Hz), 8.37 (s, 1H), 8.00 (s, 1H), 7.85 (s, 1H), 7.80 (m, 1H), 7.30 (m, 2H), 7.10 (m, 2H), 7.00 (s, 1H), 4.40 (d, 2H, J=6 Hz), 3.52 (s, 2H); MS (ESI) m/z 418 (MH⁺).

### Example 302

### 3-Chloro-6-furan-3-yl-8-trifluoromethyl-imidazo[1,2-a]pyridine-2-carboxylic Acid-2-fluoro-benzylamide (Compound 402)

Prepared using standard HATU coupling. ¹H NMR (d₆-DMSO, 300 MHz) δ 8.78 (m, 2H), 8.55 (s, 1H), 8.21 (s, 1H), 7.82 (t, 1H, J=2 Hz), 7.31 (m, 3H), 7.17 (m, 2H), 4.54 (d, J=6 Hz, 2H); MS (ESI) m/z=438 (MH⁺).

### Example 303

### 3-Chloro-6-furan-3-yl-8-trifluoromethyl-imidazo[1,2-a]pyridine-2-carboxylic Acid 3-fluorobenzamide (Compound 403)

Prepared using standard HATU coupling. ¹H NMR (d₆-DMSO, 300 MHz) δ 8.88 (t, 1H, J=6 Hz), 8.80 (s, 1H), 8.55 (s, 1H), 8.21 (s, 1H), 7.83 (t, 1H, J=2 Hz), 7.35 (m, 2H), 7.13 (m, 3H), 4.49 (d, 2H, J=6 Hz); MS (ESI) m/z=438 (MH⁺).

### Example 304

### 3-Chloro-6-furan-3-yl-8-trifluoromethyl-imidazo[1,2-a]pyridine-2-carboxylic Acid 4-fluorobenzamide (Compound 404)

Prepared using standard HATU coupling. ¹H NMR (d₆-DMSO, 300 MHz) δ 8.84 (t, 1H, J=6 Hz), 8.80 (s, 1H), 8.55 (s, 1H), 8.21 (s, 1H), 7.83 (s, 1H), 7.36 (m, 3H), 7.14 (m, 2H), 4.46 (d, 2H, J=6 Hz); MS (ESI) m/z=438 (MH⁺).

### Example 305

### 3-Chloro-6-furan-3-yl-8-trifluoromethyl-imidazo[1,2-a]-pyridine-2-carboxylic acid [2-(2-fluoro-phenyl)-ethyl]-amide (Compound 405)

Prepared using standard HATU coupling. ¹H NMR (d₆-DMSO, 300 MHz) δ 8.79 (s, 1H), 8.54 (s, 1H), 8.35 (t, 1H, J=6 Hz), 8.20 (s, 1H), 7.82 (t, 1H, 2 Hz), 7.28 (m, 3H), 7.15 (m, 2H), 3.52 (m, 2H), 2.90 (t, 2H, J=7 Hz); MS (ESI) m/z=452 (MH⁺).

### Example 306

### 3-Chloro-6-furan-3-yl-8-trifluoromethyl-imidazo[1,2-a]-pyridine-2-carboxylic acid [2-(3-fluoro-phenyl)-ethyl]-amide (Compound 406)

Prepared using standard HATU coupling. ¹H NMR (d₆-DMSO, 300 MHz) δ 8.79 (s, 1H), 8.54 (s, 1H), 8.28 (t, 1H, J=6 Hz), 8.20 (s, 1H), 7.82 (t, 1H, J=2 Hz), 7.30 (m, 2H), 7.05 (m, 3H), 3.52 (m, 2H), 2.89 (t, 2H, J=7 Hz); MS (ESI) m/z=452 (MH⁺).

### Example 307

### 3-Chloro-6-furan-3-yl-8-trifluoromethyl-imidazo[1,2-a]-pyridine-2-carboxylic acid [2-(4-fluoro-phenyl)-ethyl]-amide (Compound 407)

Prepared using standard HATU coupling. ¹H NMR (d₆-DMSO, 300 MHz) δ 8.79 (s, 1H), 8.54 (s, 1H), 8.27 (t, 1H, J=6 Hz), 8.20 (s, 1H), 7.82 (t, 1H, J=2 Hz), 7.27 (m, 3H), 7.11 (m, 2H), 3.49 (m, 2H), 2.85 (t, 2H, J=7 Hz); MS (ESI) m/z=452 (MH⁺).

### Example 308

### 3-Chloro-6-furan-3-yl-8-trifluoromethyl-imidazo[1,2-a]-pyridine-2-carboxylic Acid (2-oxo-2-phenyl-ethyl)-amide (Compound 408)

Prepared using standard HATU coupling. ¹H NMR (d₆-DMSO, 300 MHz) δ 8.82 (s, 1H), 8.56 (s, 1H), 8.48 (t, 1H, J=6 Hz), 8.24 (s, 1H), 8.04 (d, 2H, J=7 Hz), 7.83 (t, 1H, J=2 Hz), 7.68 (m, 1H), 7.56 (m, 2H), 7.31 (br s, 1H), 4.84 (d, 2H, J=6 Hz); MS (ESI) m/z 448 (MH⁺).

### Example 309

### 3-Chloro-6-furan-3-yl-8-trifluoromethyl-imidazo[1,2-a]pyridine-2-carboxylic Acid[2-(3-fluoro-phenyl)-2-oxo-ethyl]-amide (Compound 409)

Prepared using standard HATU coupling. ¹H NMR (d₆-DMSO, 300 MHz) δ 8.81 (s, 1H), 8.55 (s, 1H), 8.50 (t, 1H, J=6 Hz), 8.23 (s, 1H), 7.86 (m, 1H), 7.83 (m, 2H), 7.58 (m, 2H), 7.31 (br s, 1H), 4.82 (m, 2H); MS (ESI) m/z 466 (MH⁺).

### Example 310

### 3-Chloro-6-furan-3-yl-8-trifluoromethyl-imidazo[1,2-a]pyridine-2-carboxylic Acid (phenyl-pyridin-2-yl-methyl)-amide (Compound 410)

Prepared using standard HATU coupling. ¹H NMR (d₆-DMSO, 300 MHz) [symbol] 6.18 (d, 1H, J=8 Hz), 8.80 (s, 1H), 8.66 (d, 1H, J=6 Hz), 8.55 (s, 1H), 8.23 (s, 1H), 7.93 (br m, 1H), 7.81 (m, 1H), 7.64 (d, 1H, J=8 Hz), 7.41 (m, 3H), 7.30 (m, 4H), 6.40 (d, 1H, J=5 Hz); MS (ESI) m/z 496.9 (MH⁺).

### Example 311

### 3-Chloro-6-furan-3-yl-8-trifluoromethyl-imidazo[1,2-a]pyridine-2-carboxylic Acid (1-phenyl-ethyl)-amide (Compound 411)

Prepared using standard HATU coupling. ¹H NMR (d₆-DMSO, 300 MHz) [symbol] 8.79 (s, 1H), 8.54 (s, 1H), 8.41 (d, 1H, J=8 Hz), 8.20 (s, 1H), 7.81 (t, 1H, J=2 Hz), 7.42 (m, 2H), 7.34-7.22 (m, 4H), 5.18 (m, 1H), 1.54 (d, 3H, J=7 Hz); MS (ESI) m/z 433.9 (MH⁺).

### Example 312

### 3-Chloro-6-furan-3-yl-8-trifluoromethyl-imidazo[1,2-a]pyridine-2-carboxylic Acid (1-phenyl-ethyl)-amide (Compound 412)

Prepared using standard HATU coupling. ¹H NMR (d₆-DMSO, 300 MHz) [symbol] 8.79 (s, 1H), 8.53 (s, 1H), 8.41 (d, 1H, J=8 Hz), 8.20 (s, 1H), 7.81 (t, 1H, J=2 Hz), 7.41 (m, 2H), 7.35-7.22 (m, 4H), 5.18 (m, 1H), 1.54 (d, 3H, J=7 Hz); MS (ESI) m/z 433.9 (MH⁺).

### Example 313

### 3-Chloro-6-furan-3-yl-8-trifluoromethyl-imidazo[1,2-a]pyridine-2-carboxylic Acid (2-phenyl-propyl)-amide (Compound 413)

Prepared using standard HATU coupling. ¹H NMR (d₆-DMSO, 300 MHz) [symbol] 8.77 (s, 1H), 8.52 (s, 1H), 8.18 (s, 1H), 8.07 (t, 1H, J=6 Hz), 7.80 (s, 1H), 7.25 (m, 5H), 7.18 (m, 1H), 3.45 (m, 2H), 3.10 (m, 1H), 1.20 (d, 3H, J=7 Hz); MS (ESI) m/z 448 (MH⁺).

### Example 314

### 3-Chloro-6-furan-3-yl-8-trifluoromethyl-imidazo[1,2-a]pyridine-2-carboxylic Acid (2-phenyl-propyl)-amide (Compound 414)

Prepared using standard HATU coupling. ¹H NMR (d₆-DMSO, 300 MHz) [symbol] 8.77 (s, 1H), 8.53 (s, 1H), 8.18 (s, 1H), 8.08 (t, 1H, J=6 Hz), 7.81 (t, 1H, 2 Hz), 7.28 (m, 5H), 7.21 (m, 1H), 3.44 (m, 1H), 3.10 (m, 1H), 1.20 (d, 3H, J=7 Hz); MS (ESI) m/z 448 (MH⁺).

### Example 315

### 3-Chloro-3-furan-3-yl-8-trifluomethyl-imidazo[1,2-a]-pyridine-2-carboxylic Acid (thiazol-2-ylmethyl)-amide (Compound 415)

Prepared using standard HATU coupling. ¹H NMR (d₆-DMSO, 300 MHz) [symbol] 9.11 (t, 1H, J=6 Hz), 8.80 (s, 1H), 8.54 (s, 1H), 8.21 (s, 1H), 7.82 (s, 1H), 7.71 (d, 1H, J=7 Hz), 7.60 (d, 1H, J=7 Hz), 7.30 (br s, 1H), 4.78 (d, 2H, 6 Hz); MS (ESI) m/z 427 (MH⁺).

### Example 316

### 1-(3-Chloro-6-furan-3-yl-8-trifluoromethyl-imidazo[1,2-a]pyridine-2-carbonyl)-pyrrolidine-3-carbonitrile (Compound 416)

Prepared using standard HATU coupling. ¹H NMR (d₆-DMSO, 300 MHz) [symbol] 8.81 (s, 1H), 8.55 (s, 1H), 8.20 (s, 1H), 8.72 (br s, 1H), 7.31 (s, 1H), 4.09 (m, 1H), 3.89 (m, 1H), 3.75-3.48 (m, 3H), 2.25 (m, 2H); MS (ESI) m/z=409 (MH⁺).

### Example 317

### (3-Chloro-6-furan-3-yl-8-trifluoromethyl-imidazo[1,2-a]pyridine-2-yl)-(3-[1,2,4]oxadiazol-3-yl-pyrrolidin-1-yl)-methanone (Compound 417)

### Step 1: 1-(3-Chloro-6-furan-3-yl-8-trifluoromethyl-imidaz [1,2-a]pyridine-2-carbonyl)-N-hydroxy-pyrrolidine-3-carboxamidine

1-(3-Chloro-6-furan-3-yl-8-trifluoromethyl-imidazo[1,2-a]pyridine-2-carbonyl)-pyrrolidine-3-carbonitrile, (compound 416, 89 mg, 0.22 mmol) was suspended in anhydrous ethanol (4 mL). To this suspension was added NH₂OH (50% in H₂O, 0.1 mL) and the reaction mixture was heated at 80° C. for 1 hour. The resulting mixture was evaporated to dryness to give crude 1-(3-chloro-6-furan-3-yl-8-trifluoromethyl-imidazo[1,2-a]pyridine-2-carbonyl)-N-hydroxy-pyrrolidine-3-carboxamidine (92 mg, 95.8%) which was used for the next step without further purification. MS (ESI) m/z=442 (MH⁺).

### Step 2: (3-Chloro-6-furan-3-yl-8-trifluoromethyl-imidazo[1,2-a]pyridine-2-yl)-(3-[1,2,4]oxadiazol-3-yl-pyrrolidin-1-yl)-methanone (compound 417)

To a stirred suspension of 1-(3-chloro-6-furan-3-yl-8-trifluoromethyl-imidazo[1,2-a]pyridine-2-carbonyl)-N-hydroxy-pyrrolidine-3-carboxamidine (92 mg, 0.21 mmol) in trimethyl orthoformate (4 mL) was added boron trifluoride diethyl etherate (4 drops). The mixture was heated at 100° C. for 30 minutes. Reaction mixture was evaporated under reduced pressure followed by purification using reverse phase HPLC to give (3-chloro-6-furan-3-yl-8-trifluoromethyl-imidazo[1,2-a]pyridine-2-yl)-(3-[1,2,4]oxadiazol-3-yl-pyrrolidin-1-yl)-methanone (47 mg). ¹H NMR (d₆-DMSO, 300 MHz) [symbol] 9.57 (s, 0.5H), 9.53 (s, 0.5H), 8.80 (s, 1H), 8.52 (s, 1H), 8.18 (s, 1H), 7.82 (s, 1H), 7.29 (s, 1H), 4.26 (m, 0.5H), 4.02 (m, 2H), 3.69 (m, 2.5H), 2.36 (m, 1H), 2.17 (m, 1H); MS (ESI) m/z=452 (MH⁺).

### Example 318

### (3-Chloro-6-furan-3-yl-8-trifluoromethyl-imidazo[1,2-a]pyridine-2-yl)-[3-(1H-tetrazol-5-yl)-pyrrolidin-1-yl]-methanone (Compound 418)

Prepared using a similar method as in Example 215 (compound 315) ¹H NMR (d₆-DMSO, 300 MHz) [symbol] 8.80 (s, 1H), 8.54 (s, 1H), 8.19 (s, 1H), 7.82 (s, 1H), 7.30 (br s, 1H), 4.31 (m, 0.5H), 4.01 (m, 2H), 3.87 (m, 1H), 3.72 (m, 1.5H), 2.42 (m, 1H), 2.19 (m, 1H); MS (ESI) m/z=452 (MH⁺).

### Example 319

### 3-[1-(3-Chloro-6-furan-3-yl-8-trifluoromethyl-imidazo[1,2-a]pyridine-2-carbonyl)-pyrrolodin-3-yl]-4H-[1,2,4]oxodiazol-5-one (Compound 419)

Prepared using a similar method as in Example Example 210 (compound 310). ¹H NMR (d₆-DMSO, 300 MHz) [symbol] 8.80 (s, 1H), 8.54 (s, 1H), 8.19 (s, 1H), 7.82 (t, 1H, J=2 Hz), 7.30 (br s, 1H), 7.14 (m, 0.5H), 3.92 (m, 2H), 3.56 (m, 2.5H), 2.26 (m, 1H), 2.12 (m, 1H); MS (ESI) m/z 468 (MH⁺).

### Example 320

### (3-Chloro-6-furan-3-yl-8-trifluoromethyl-imidazo[1,2-a]pyridine-2-yl)-[3-(3,4-difluoro-phenyl)-pyrrolidin-1-yl]-methanone (Compound 420)

Prepared using standard HATU coupling. ¹H NMR (d₆-DMSO, 300 MHz) [symbol] 8.80 (d, 1H, J=5 Hz), 8.53 (d, 1H, J=4 Hz), 8.17 (d, 1H, J=4 Hz), 7.81 (br s, 1H), 7.34-7.19 (m, 5H), 4.26 (m, 0.5H), 4.04 (m, 1H), 3.86-3.40 (m, 3.5H), 2.29 (m, 1H), 2.09 (m, 1H); MS (ESI) m/z 496 (MHt).

### Example 321

### 1-(3-Chloro-6-furan-3-yl-8-trifluoromethyl-imidazo[1,2-a]pyridine-2-carbonyl)-pyrrolidine-3-carboxylic acid cyclopropylamide (Compound 421)

### Step 1: 1-(3-Chloro-6-furan-3-yl-8-trifluoromethyl-imidazo[1,2-a]pyridine-2-carbonyl)-pyrrolidine-3-carboxylic Acid Methyl Ester

3-Chloro-6-furan-3-yl-8-trifluoromethyl-imidazo[1,2-a]pyridine-2-carboxylic acid, (750 mg, 2.3 mmol) and pyrrolidine-3-carboxylic aid methyl ester HCl salt, (376 mg, 2.3 mmol) reacted using standard HATU coupling conditions to give 1-(3-chloro-6-furan-3-yl-8-trifluoromethyl-imidazo[1,2-a]pyridine-2-carbonyl)-pyrrolidine-3-carboxylic acid methyl ester (0.89 g, 88%). MS (ESI) m/z 442 (MH⁺).

### Step 2: 3-Chloro-6-furan-3-yl-8-trifluoromethyl-imidazo[1,2-a]pyridine-2-carbonyl)-pyrrolidine-3-carboxylic Acid, (IS2516-71)

1-(3-Chloro-6-furan-3-yl-8-trifluoromethyl-imidazo[1,2-a]pyridine-2-carbonyl)-pyrrolidine-3-carboxylic acid methyl ester (0.89 g, 2.0 mmol) was dissolved in THF/MeOH/H₂O (3:1:1 v/v, 20 mL). To this solution was added LiOH—H₂O (0.26 g, 6.0 mmol). Reaction mixture was stirred at room temperature for 2 hours. The organic solvents were removed and the remaining aqueous solution was acidified using 1M HCl. The solids were filtered, washed using additional H₂O, and dried to give 3-chloro-6-furan-3-yl-8-trifluoromethyl-imidazo[1,2-a]pyridine-2-carbonyl)-pyrrolidine-3-carboxylic acid (0.67 g, 79%). MS (ESI) m/z 423 (MH⁺).

### Step 3: 1-(3-Chloro-6-furan-3-yl-8-trifluoromethyl-imidazo[1,2-a]pyridine-2-carbonyl)-pyrrolidine-3-carboxylic Acid Cyclopropylamide (compound 421)

Prepared using standard HATU coupling of the above acid and cyclopropylamine. 1H NMR (d₆-DMSO, 300 MHz) [symbol] 8.46 (s, 1H), 8.20 (s, 1H), 7.84 (s, 1H), 7.48 (t, 1H, J=2 Hz), 6.96 (s, 1H), 3.63-3.16 (m, 5H), 2.56 (m, 1H), 2.28 (m, 1H), 1.68 (m, 2H), 0.26 (m, 2H), 0.03 (m, 2H); MS (ESI) m/z 467 (MH⁺).

### Example 322

### (3-Chloro-6-furan-3-yl-8-trifluoromethyl-imidazo[1,2-a]pyridin-2-yl)-(3-thiophen-2-yl-2,5-dihydro-pyrrol-1-yl)-methanone (Compound 422)

### Step 1: 3-Trifluoromethanesulfonyloxy-2,5-dihydro-pyrrole-1-carboxylic Acid Tert-butyl Ester

To a solution of lithium diisopropylamide (2M in heptane/THF/ethylbenzene, 6.5 mL, 12.96 mmol) in THF (30 mL) at −78° C. was added a solution of N-Boc-3-pyrrolidinone (2 g, 10.8 mmol) in THF (30 mL) over 10 min. After 40 min, a solution of N-phenylbis(trifluoromethanesulfinimide) (4.24 g, 11.88 mmol) in THF (30 mL) was added. After 3 hours, the mixture was quenched with saturated aqueous solution of NaHCO₃ and diluted with ethyl ether (250 mL). The aqueous phase was discarded and the organic phase was washed with 5% citric acid (2×50 mL), 10% aq NaOH (2×50 mL), water (50 mL), and brine (50 mL). The organic phase was dried (Na₂SO₄), filtered and concentrated. The crude product was absorbed on silica gel followed by column chromatography [n-hex/EtOAc (15:1 v/v) followed by n-hex/EtOAc (9:1 v/v)] gave 3-trifluoromethanesulfonyloxy-2,5-dihydro-pyrrole-1-carboxylic acid tert-butyl ester (1.2 g, 35%) as an oil. ¹H NMR (d₆-DMSO, 300 MHz) [symbol] 1.42 (s, 9H), 4.06-4.26 (m, 4H), 6.02-6.18 (m, 1H); MS (ESI) m/z=262 (MH⁺-^{t}Bu).

### Step 2: 3-Thiophen-2-yl-2,5-dihydro-pyrrole-1-carboxylic Acid Tert-butyl Ester

To a solution of 3-trifluoromethanesulfonyloxy-2,5-dihydro-pyrrole-1-carboxylic acid tert-butyl ester (184.5 mg, 0.582 mmol) in THF (3 mL) was added 2-thienylzinc bromide (0.5 M in THF, 1.16 mL, 0.582 mmol) and tetrakis(triphenylphosphine)palladium(0) (67.2 mg, 0.058 mmol). The mixture was heated at 50° C. for 105 min. Upon cooling, the mixture was filtered warm and diluted with EtOAc (50 mL) and washed with brine (20 mL). The organic layer was dried (Na₂SO₄), filtered and concentrated. Column chromatography [n-hex/EtOAc (12:1 v/v)] of the crude gave 3-thiophen-2-yl-2,5-dihydro-pyrrole-1-carboxylic acid tert-butyl ester (49 mg, 33%) as an oil. ¹H NMR (d₆-DMSO, 300 MHz) [symbol] 1.44 (s, 4.5H), 1.45 (s, 4.5H), 4.17 (m, 2H), 4.36 (m, 2H), 6.08 (brd, 1H, J=12.3 Hz), 7.05 (t, 1H, J=3.2 hz), 7.11 (d, 1H, J=3.2 Hz), 7.51 (d, 1H, J=5.3 Hz); MS (ESI) m/z=274 (MNa⁺).

### Step 3: 3-Thiophen-2-yl-2,5-dihydro-1H-pyrrole

A solution of 3-thiophen-2-yl-2,5-dihydro-pyrrole-1-carboxylic acid tert-butyl ester (45.5 mg, 0.181 mmol) was stirred in 30% TFA/DCM solution (10 mL). After 50 min, the solvents were removed and evaporated with toluene (2×3 mL) to give 3-thiophen-2-yl-2,5-dihydro-1H-pyrrole (49 mg) as a brown solid which was used for the next step without further purification. ¹H NMR (d₆-DMSO, 300 MHz) [symbol] 4.12 (brs 2H), 4.31 (brs, 2H), 6.13 (m, 1H), 7.10 (dd, 1H, J=3.5, 5 Hz), 7.21 (dd, 1H, J=0.6, 5 Hz), 7.60 (dd, 1H, J=0.9, 5 Hz), 9.33 (brs, 2H); MS (ESI) m/z=152.1 (MH⁺).

### Step 4: (3-Chloro-6-furan-3-yl-8-trifluoromethyl-imidazo[1,2-a]pyridin-2-yl)-(3-thiophen-2-yl-2,5-dihydro-pyrrol-1-yl)-methanone (compound 422)

Prepared using standard HATU coupling of the above amine. ¹H NMR (d₆-DMSO, 300 MHz) [symbol] 4.51 (m, 1H), 4.70 (m, 1H), 4.82 (m, 1H), 6.22 (m, 1H), 5.04 (m, 1H), 7.01 (dd, 0.5H, J=0.9, 2.6 Hz), 7.08 (dd, 0.5H, J=2.6, 3.5 Hz), 7.10 (dd, 0.5H, J=2.5, 3.8 Hz), 7.21 (brd, 0.5H, J=2.5 Hz), 7.32-7.35 (m, 1H), 7.53 (dd, 0.5H, J=1.2, 3.3 Hz), 7.55 (dd, 0.5H, J=0.9, 2.3 Hz), 7.83-7.86 (m, 1H), 8.24-8.26 (brs, 1H), 8.57 (brs, 1H), 8.85 (s, 1H); MS (ESI) m/z=464 (MH⁺).

### Example 323

### (3-Chloro-6-furan-3-yl-8-trifluoromethyl-imidazo[1,2-a]pyridin-2-yl)-(3-thiophen-2-yl-pyrrolidin-1-yl)-methanone (Compound 423)

### Step 1: 3-Thiophen-2-yl-pyrrolidine

A suspension of 3-thiophen-2-yl-2,5-dihydro-pyrrole-1-carboxylic acid tert-butyl ester (Example 322, Step 2, (147 mg, 0.585 mmol) and 10% Pd/C (100 mg) was stirred under H₂ in MeOH. After 24 hours, the catalyst was filtered and the solvent concentrated under reduced pressure. Column chromatography [n-hex/EtOAc (9:1 v/v)] of the crude gave 3-thiophen-2-yl-pyrrolidine-1-carboxylic acid tert-butyl ester (138 mg, 93%) as oil. A solution of the above compound (136 mg, 0.537 mmol) was stirred in 30% TFA/DCM (10 mL). After 30 min, the solvents were removed and evaporated with toluene (2×2 mL) to give 3-thiophen-2-yl-pyrrolidine (187 mg) which was used for the next step without further purification. ¹H NMR (d₆-DMSO, 300 MHz) [symbol] 2.34-2.46 (m, 1H), 1.89-2.08 (m, 1H), 3.00-3.80 (m, 5H), 7.01 (dd, 1H, J=3.5, 5 Hz), 7.04 (dt, 1H, J=1.2, 3.5 Hz), 7.45 (dd, 1H, J=1.5, 5 Hz), 8.93 (brs, 2H); MS (ESI) m/z=154.1 (MH⁺).

### Step 2: 3 (3-Chloro-6-furan-3-yl-8-trifluoromethyl-imidazo[1,2-a]pyridin-2-yl)-(3-thiophen-2-yl-pyrrolidin-1-yl)-methanone (compound 423)

Prepared using standard HATU coupling of the above amine. ¹H NMR (d₆-DMSO, 300 MHz) [symbol] 2.32-2.44 (m, 1H), 1.96-2.13 (m, 1H), 3.43-4.34 (m, 5H), 6.94-7.04 (m, 2H), 7.32 (m, 1H), 7.38 (dd, 0.5H, J=1.8, 3.5 Hz), 7.41 (dd, 0.5H, J=3.5, 5 Hz), 7.83 (t, 0.5H, J=1.8 Hz), 7.84 (t, 0.5H, J=1.8 Hz), 8.19 (brs, 0.5H), 8.21 (brs, 0.5H), 8.55 (m, 1H), 8.12 (s, 0.5H), 8.22 (s, 0.5H); MS (ESI) m/z=466 (MH⁺).

### Example 324

### (3-Chloro-6-furan-3-yl-8-trifluoromethyl-imidazo[1,2-a]pyridin-2-yl)-[3-(2-fluoro-phenyl)-2,5-dihydro-pyrrol-1-yl]-methanone (Compound 424)

### Step 1: 3-(2-Fluoro-phenyl)-2,5-dihydro-1H-pyrrole

Similar to the preparation of 3-thiophen-2-yl-2,5-dihydro-1H-pyrrole (Example 322, Step 2 and 3,3-trifluoromethanesulfonyloxy-2,5-dihydro-pyrrole-1-carboxylic acid tert-butyl ester reacted with 2-fluorophenylboronic acid, Pd(PPh₃)₄ under standard Suzuki conditions gave 3-(2-fluoro-phenyl)-2,5-dihydro-pyrrole-1-carboxylic acid tert-butyl ester which was hydrolyzed with 30% TFA/DCM to give 3-(2-fluoro-phenyl)-2,5-dihydro-1H-pyrrole. ¹H NMR (d₆-DMSO, 300 MHz) [symbol] 4.17 (brs, 2H), 4.38 (brs, 2H), 6.44 (m, 1H), 7.23-7.47 (m, 3H), 7.52 (dt, 1H, J=1.8, 8 Hz), 9.38 (brs, 2H); MS (ESI) m/z=164 (MH⁺).

### Step 2: (3-Chloro-6-furan-3-yl-8-trifluoromethyl-imidazo[1,2-a]pyridin-2-yl)-[3-(2-fluoro-phenyl)-2,5-dihydro-pyrrol-1-yl]-methanone (compound 424)

Prepared using standard HATU coupling of the above amine. ¹H NMR (d₆-DMSO, 300 MHz) [symbol] 4.49 (m, 1H), 4.71 (m, 1H), 4.83 (m, 1H), 5.05 (m, 1H), 6.46 (brs, 1H), 7.14-7.37 (m, 4H), 7.45-7.52 (m, 1H), 7.78 (t, 1H, J=1.8 Hz), 8.16 (d, 1H, J=1.2 Hz), 8.51 (s, 1H), 8.78 (s, 1H); MS (ESI) m/z=476 (MH⁺).

### Example 325

### (3-Chloro-6-furan-3-yl-8-trifluoromethyl-imidazo[1,2-a]pyridin-2-yl)-(3-thiophen-3-yl-2,5-dihydro-pyrrol-1-yl)-methanone (Compound 425)

### Step 1: 3-Thiophen-3-yl-2,5-dihydro-1H-pyrrole

Similar to the preparation of 3-thiophen-2-yl-2,5-dihydro-1H-pyrrole (Example 322, Step 2 and 3,3-trifluoromethanesulfonyloxy-2,5-dihydro-pyrrole-1-carboxylic acid tert-butyl ester reacted with 3-thienylboronic acid, Pd(PPh₃)₄ under standard Suzuki conditions gave 3-thiophen-3-yl-2,5-dihydro-pyrrole-1-carboxylic acid tert-butyl ester which was hydrolyzed with 30% TFA/DCM to give 3-thiophen-3-yl-2,5-dihydro-1H-pyrrole. ¹H NMR (d₆-DMSO, 300 MHz) [symbol] 4.12 (m, 2H), 4.27 (m, 2H), 6.25 (m, 1H), 7.46 (dd, 1H, J=2.6, 3.8 Hz), 7.62-7.65 (m, 2H), 9.30 (brs, 2H); MS (ESI) m/z=152 (MH⁺).

### Step 2: (3-Chloro-6-furan-3-yl-8-trifluoromethyl-imidazo[1,2-a]pyridin-2-yl)-(3-thiophen-3-yl-2,5-dihydro-pyrrol-1-yl)-methanone (compound 425)

Prepared using standard HATU coupling of the above amine. ¹H NMR (d₆-DMSO, 300 MHz) [symbol] 4.50 (m, 1H), 4.68 (m, 1H), 4.81 (m, 1H), 4.95 (m, 1H), 6.28-6.34 (m, 1H), 7.27-7.47 (m, 2H), 7.57-7.62 (m, 2H), 7.84-7.86 (m, 1H), 8.20-8.26 (m, 1H), 8.57 (m, 1H), 8.85 (brs, 1H); MS (ESI) m/z=463.9 (MH⁺).

### Example 326

### (3-Chloro-6-furan-3-yl-8-trifluoromethyl-imidazo[1,2-a]pyridin-2-yl)-[3-(3-fluoro-phenyl)-2,5-dihydro-pyrrol-1-yl]-methanone (Compound 426)

### Step 1: 3-(3-Fluoro-phenyl)-2,5-dihydro-1H-pyrrole

Similar to the preparation of 3-thiophen-2-yl-2,5-dihydro-1H-pyrrole (Example 322, Step 2 and 3,3-trifluoromethanesulfonyloxy-2,5-dihydro-pyrrole-1-carboxylic acid tert-butyl ester reacted with 3-fluorophenylboronic acid, Pd(PPh₃)₄ under standard Suzuki conditions gave 3-(3-fluoro-phenyl)-2,5-dihydro-pyrrole-1-carboxylic acid tert-butyl ester which was hydrolyzed with 30% TFA/DCM to give 3-(3-fluoro-phenyl)-2,5-dihydro-1H-pyrrole. ¹H NMR (d₆-DMSO, 300 MHz) [symbol] 4.16 (m, 2H), 4.35 (m, 2H), 6.55 (m, 1H), 7.16-7.50 (m, 4H), 9.36 (brs, 2H); MS (ESI) m/z=164.1 (MH⁺).

### Step 2: (3-Chlo ro-6-furan-3-yl-8-trifluoromethyl-imidazo[1,2-a]pyridin-2-yl)-[3-(3-fluoro-phenyl)-2,5-dihydro-pyrrol-1-yl]-methanone (Compound 426)

Prepared using standard HATU coupling of the above amine. ¹H NMR (d₆-DMSO, 300 MHz) [symbol] 4.56 (m, 1H), 4.74 (m, 1H), 4.87 (m, 1H), 5.04 (m, 1H), 6.58-6.65 (m, 1H), 7.12-7.64 (m, 5H), 7.84-7.78 (m, 1H), 8.22-8.26 (m, 1H), 8.58 (s, 1H), 8.85 (s, 1H); MS (ESI) m/z=476 (MH⁺).

### Example 327

### (3-Chloro-6-furan-3-yl-8-trifluoromethyl-imidazo[1,2-a]pyridin-2-yl)-[3-(4-fluoro-phenyl)-2,5-dihydro-pyrrol-1-yl]-methanone (Compound 427)

### Step 1: 3-(4-Fluoro-phenyl)-2,5-dihydro-1H-pyrrole

Similar to the preparation of 3-thiophen-2-yl-2,5-dihydro-1H-pyrrole (Example 322, Step 2 and 3,3-trifluoromethanesulfonyloxy-2,5-dihydro-pyrrole-1-carboxylic acid tert-butyl ester reacted with 4-fluorophenylboronic acid, Pd(PPh₃)₄ under standard Suzuki conditions gave 3-(4-fluoro-phenyl)-2,5-dihydro-pyrrole-1-carboxylic acid tert-butyl ester which was hydrolyzed with 30% TFA/DCM to give 3-(4-fluoro-phenyl)-2,5-dihydro-1H-pyrrole. ¹H NMR (d₆-DMSO, 300 MHz) [symbol] 4.15 (m, 2H), 4.34 (m, 2H), 6.41 (m, 1H), 7.22-7.30 (m, 2H), 7.56-7.64 (m, 2H), 9.38 (brs, 2H); MS (ESI) m/z=164 (MH⁺).

### Step 2: (3-Chloro-6-furan-3-yl-8-trifluoromethyl-imidaz[1,2-a]pyridin-2-yl)-[3-(4-fluoro-phenyl)-2,5-dihydro-pyrrol-1-yl]-methanone (compound 427)

Prepared using standard HATU coupling of the above amine. ¹H NMR (d₆-DMSO, 300 MHz) [symbol] 4.53 (m, 1H), 4.73 (m, 1H), 4.85 (m, 1H), 5.02 (m, 1H), 6.48 (m, 1H), 7.20-7.64 (m, 5H), 7.84-7.87 (m, 1H), 8.22-8.26 (m, 1H), 8.57 (s, 1H), 8.85 (s, 1H); MS (ESI) m/z=475.9 (MH⁺).

### Example 328

### (3-Chloro-6-furan-3-yl-8-trifluoromethyl-imidazo[1,2-a]pyridin-2-yl)-(3-thiazol-2-yl-2,5-dihydro-pyrrol-1-yl)-methanone (Compound 428)

### Step 1: 2-(2,5-Dihydro-1H-pyrrol-3-yl)-thiazole

Similar to the preparation of 3-thiophen-2-yl-2,5-dihydro-1H-pyrrole (Example 322, Step 2 and 3,3-trifluoromethanesulfonyloxy-2,5-dihydro-pyrrole-1-carboxylic acid tert-butyl ester reacted with 2-thiazolylzinc bromide, Pd(PPh₃)₄ under similar Negishi conditions gave 3-thiazol-2-yl-2,5-dihydro-pyrrole-1-carboxylic acid tert-butyl ester which was hydrolyzed with PGP PORTFA/DCM to give 2-(2,5-dihydro-1H-pyrrol-3-yl)-thiazole. ¹H NMR (d₆-DMSO, 300 MHz) [symbol] 4.20 (m, 2H), 4.40 (m, 2H), 6.65 (m, 1H), 7.84 (d, 1H, J=3.2 Hz), 7.90 (d, 1H, J=3.2 Hz), 9.47 (brs, 2H); MS (ESI) m/z=153 (MH⁺).

### Step 2: (3-Chloro-6-furan-3-yl-8-trifluoromethyl-imidazo[1,2-a]pyridin-2-yl)-(3-thiazol-2-yl-2,5-dihydro-pyrrol-1-yl)-methanone (compound 428)

Prepared using standard HATU coupling of the above amine. ¹H NMR (d₆-DMSO, 300 MHz) [symbol] 4.58 (m, 1H), 4.78 (m, 1H), 4.93 (m, 1H), 5.11 (m, 1H), 6.68-6.75 (m, 1H), 7.34 (m, 1H), 7.77 (d, 0.5H, J=3.2 Hz), 7.80 (d, 0.5H, J=3.2 Hz), 7.84-7.87 (m, 1.5H), 7.90 (d, 0.5H, J=3.2 Hz), 8.24 (s, 1H), 8.50 (s, 1H), 8.85 (s, 1H); MS (ESI) m/z=464.9 (MH⁺).

### Example 329

### (3-Chloro-6-furan-3-yl-8-trifluoromethyl-imidazo[1,2-a]pyridin-2-yl)-(3-furan-3-yl-2,5-dihydro-pyrrol-1-yl)-methanone (Compound 429)

### Step 1: 3-Furan-3-yl-2,5-dihydro-1H-pyrrole

Similar to the preparation of 3-thiophen-2-yl-2,5-dihydro-1H-pyrrole (Example 322, Step 2 and 3,3-trifluoromethanesulfonyloxy-2,5-dihydro-pyrrole-1-carboxylic acid tert-butyl ester reacted with 3-furanboronic acid, Pd(PPh₃)₄ under standard Suzuki conditions gave 3-furan-3-yl-2,5-dihydro-pyrrole-1-carboxylic acid tert-butyl ester which was hydrolyzed with 30% TFA/DCM to give 3-furan-3-yl-2,5-dihydro-1H-pyrrole. ¹H NMR (d₆-DMSO, 300 MHz) [symbol] 4.08 (m, 2H), 4.15 (m, 2H), 6.13 (brs, 1H), 6.85 (t, 1H, J=1 Hz), 7.73 (t, 1H, J=1.7 Hz), 7.90 (s, 1H), 9.28 (brs, 2H); MS (ESI) m/z=136.3 (MH⁺).

### Step 2: (3-Chloro-6-furan-3-yl-8-trifluoromethyl-imidaz[1,2-a]pyridin-2-yl)-(3-furan-3-yl-2,5-dihydro-pyrrol-1-yl)-methanone (Compound 429)

Prepared using standard HATU coupling of the above amine. ¹H NMR (d₆-DMSO, 300 MHz) [symbol] 4.47 (m, 1H), 4.57 (m, 1H), 4.77 (m, 1H), 4.82 (m, 1H), 6.18 (t, 0.5H, J=1.8 Hz), 6.20 (t, 0.5H, J=1.8 Hz), 6.79 (dd, 0.5H, J=0.9, 1.8 Hz), 6.82 (dd, 0.5H, J=0.9, 1.8 Hz), 7.34 (m, 1H), 7.58 (s, 0.5H), 7.70-7.73 (m, 1H), 7.85 (m, 1H), 7.92 (s, 0.5H), 8.20-8.25 (m, 1H), 8.57 (d, 1H, J=1.2 Hz), 8.84 (s, 1H); MS (ESI) m/z=447.9 (MH⁺).

### Example 330

### (3-Chloro-6-furan-3-yl-8-trifluoromethyl-imidazo[1,2-a]pyridin-2-yl)-(3-thiazol-2-yl-pyrrolidin-1-yl)-methanone (Compound 430)

### Step 1: 2-Pyrrolidin-3-yl-thiazole

Using similar method as for the preparation of 3-thiophen-2-yl-pyrrolidine (Example 323, Step 1,3-thiazol-2-yl-2,5-dihydro-pyrrole-1-carboxylic acid tert-butyl ester was reduced followed by acid hydrolysis to give 2-pyrrolidin-3-yl-thiazole. ¹H NMR (d₆-DMSO, 300 MHz) [symbol] 8.06-2.18 (m, 1H), 2.37-2.50 (m, 1H), 3.20-4.06 (m, 5H), 7.71 (d, 1H, J=3.2 Hz), 7.78 (d, 1H, J=3.2 Hz), 8.97 (brs, 2H); MS (ESI) m/z=155.3 (MH⁺).

### Step 2: 3 (3-Chloro-6-furan-3-yl-8-trifluoromethyl-imidaz [1,2-a]pyridin-2-yl)-(3-thiazol-2-yl-pyrrolidin-1-yl)-methanone (Compound 430)

Prepared using standard HATU coupling of the above amine. ¹H NMR (d₆-DMSO, 300 MHz) [symbol] 2.12-2.28 (m, 1H), 2.36-2.50 (m, 1H), 3.60-4.10 (m, 4.5H), 4.31 (dd, 0.5H, J=7, 11.4 Hz), 7.33 (m, 1H), 7.64 (d, 0.5H, J=3.2 Hz), 7.68 (d, 0.5H, J=3.2 Hz), 7.74 (d, 0.5H, J=3.2 Hz), 7.77 (d, 0.5H, J=3.2 Hz), 7.83 (t, 0.5H, J=1.8 Hz), 7.84 (t, 0.5H, J=1.4 Hz), 8.18-8.22 (m, 1H), 8.56 (s, 1H), 8.82 (s, 1H); MS (ESI) m/z=466.9 (MH⁺).

### Example 331

### (3-Chloro-6-furan-3-yl-8-trifluoromethyl-imidazo[1,2-a]pyridin-2-yl)-[3-(tetrahydro-furan-3-yl)-pyrrolidin-1-yl]-methanone (compound 431)

### Step 1: 3-(Tetrahydro-furan-3-yl)-pyrrolidine

Using similar method as for the preparation of 3-thiophen-2-yl-pyrrolidine (Example 323, Step 1,3-furan-3-yl-2,5-dihydro-pyrrole-1-carboxylic acid tert-butyl ester was reduced for 2 days followed by acid hydrolysis to give 3-(tetrahydro-furan-3-yl)-pyrrolidine. ¹H NMR (d₆-DMSO, 300 MHz) [symbol] 1.40-2.20 (m, 6H), 3.50-3.80 (m, 4H), 2.60-3.20 (m, 4H), 8.64 (brs, 2H); MS (ESI) m/z=141.9 (MH⁺).

### Step 2: (3-Chloro-6-furan-3-yl-8-trifluoromethyl-imidazo[1,2-a]pyridin-2-yl)-[3-(tetrahydro-furan-3-yl)-pyrrolidin-1-yl]-methanone (Compound 431)

Prepared using standard HATU coupling of the above amine. ¹H NMR (d₆-DMSO, 300 MHz) [symbol] 1.40-2.20 (m, 6H), 3.06-4.06 (m, 8H), 7.32 (dd, 1H, J=0.9, 1.8 Hz), 7.84 (t, 1H, J=1.8 Hz), 8.19 (s, 1H), 8.55 (s, 1H), 8.81 (s, 1H); MS (ESI) m/z=454.1 (MH⁺).

### Example 332

### [3-Bromo-6-(1H-pyrazol-4-yl)-8-trifluoromethyl-imidazo[1,2-a]pyridin-2-yl]-(3-thiazol-2-yl-pyrrolidin-1-yl)-methanone (Compound 432)

Under standard HATU coupling conditions, 3-bromo-6-(1H-pyrazol-4-yl)-8-trifluoromethyl-imidazo[1,2-a]pyridine-2-carboxylic acid, and 2-pyrrolidin-3-yl-thiazole (Example 330, Step 1) gave [3-bromo-6-(1H-pyrazol-4-yl)-8-trifluoromethyl-imidazo[1,2-a]pyridin-2-yl]-(3-thiazol-2-yl-pyrrolidin-1-yl)-methanone. ¹H NMR (d₆-DMSO, 300 MHz) [symbol] 2.15-2.25 (m, 1H), 2.37-2.49 (m, 1H), 3.59-4.10 (m, 4.5H), 4.26 (dd, 0.5H, J=6.5, 10.8 Hz), 7.64 (d, 0.5H, J=3.2 Hz), 7.68 (d, 0.5H, J=3.2 Hz), 7.73 (d, 0.5H, J=3.2 Hz), 7.77 (d, 0.5H, J=3.2 Hz), 8.19 (brs, 1H), 8.39 (s, 2H), 8.75 (s, 1H), 13.15 (s, 1H); MS (ESI) m/z=511.1 (MH⁺).

### Example 333

### [3-Bromo-6-(1H-pyrazol-4-yl)-8-trifluoromethyl-imidazo[1,2-a]pyridin-2-yl]-(3-thiazol-2-yl-2,5-dihydro-pyrrol-1-yl)-methanone (Compound 433)

Under standard HATU coupling conditions, 3-bromo-6-(1H-pyrazol-4-yl)-8-trifluoromethyl-imidazo[1,2-a]pyridine-2-carboxylic acid, and 2-(2,5-dihydro-1H-pyrrol-3-yl)-thiazole (Example 328, Step 1) gave [3-bromo-6-(1H-pyrazol-4-yl)-8-trifluoromethyl-imidazo[1,2-a]pyridin-2-yl]-(3-thiazol-2-yl-2,5-dihydro-pyrrol-1-yl)-methanone. ¹H NMR (d₆-DMSO, 300 MHz) [symbol] 4.58 (m, 1H), 4.77 (m, 1H), 4.88 (m, 1H), 5.07 (m, 1H), 6.69-6.72 (m, 1H), 7.76 (d, 0.5H, J=3.2 Hz), 7.79 (d, 0.5H, J=3.5 Hz), 7.84 (d, 0.5H, J=3.2 Hz), 7.90 (d, 0.5H, J=3.2 Hz), 8.23 (s, 1H), 8.40 (s, 2H), 8.79 (s, 1H); MS (ESI) m/z=509.1 (MH⁺).

### Example 334

### [3-Bromo-6-(1H-pyrazol-4-yl)-8-trifluoromethyl-imidazo[1,2-a]pyridin-2-yl]-(3-thiophen-2-yl-pyrrolidin-1-yl)-methanone (Compound 434)

Under standard HATU coupling conditions, 3-bromo-6-(1H-pyrazol-4-yl)-8-trifluoromethyl-imidazo[1,2-a]pyridine-2-carboxylic acid, and 3-thiophen-2-yl-pyrrolidine (Example 323, Step 1) gave [3-bromo-6-(1H-pyrazol-4-yl)-8-trifluoromethyl-imidazo[1,2-a]pyridin-2-yl]-(3-thiophen-2-yl-pyrrolidin-1-yl)-methanone. ¹H NMR (d₆-DMSO, 300 MHz) [symbol] 1.96-2.12 (m, 1H), 2.32-2.46 (m, 1H), 3.45-4.26 (m, 5H), 6.94-7.05 (m, 2H), 7.38 (dd, 0.5H, J=1.8, 4.4 Hz), 7.41 (dd, 0.5H, J=1.5, 5 Hz), 8.18 (s, 0.5H), 8.20 (s, 0.5H), 8.39 (brs, 2H), 8.75 (s, 0.5H), 8.76 (s, 0.5H); MS (ESI) m/z=510 (MH⁺).

### Example 335

### [3-Bromo-6-(1H-pyrazol-4-yl)-8-trifluoromethyl-imidazo[1,2-a]pyridin-2-yl]-(3-thiophen-3-yl-2,5-dihydro-pyrrol-1-yl)-methanone (Compound 435)

Under standard HATU coupling conditions, 3-bromo-6-(1H-pyrazol-4-yl)-8-trifluoromethyl-imidazo[1,2-a]pyridine-2-carboxylic acid, and 3-thiophen-3-yl-2,5-dihydro-1H-pyrrole (Example 325, Step 1) gave [3-bromo-6-(1H-pyrazol-4-yl)-8-trifluoromethyl-imidazo[1,2-a]pyridin-2-yl]-(3-thiophen-3-yl-2,5-dihydro-pyrrol-1-yl)-methanone. ¹H NMR (d₆-DMSO, 300 MHz) [symbol] 4.50 (m, 1H), 4.68 (m, 1H), 4.76 (m, 1H), 4.90 (m, 1H), 6.27-6.34 (m, 1H), 7.29 (dd, 0.5H, J=1.5, 2.5 Hz), 7.40 (dd, 0.5H, J=1.5, 5.2 Hz), 7.45 (dd, 0.5H, J=2.5, 4 Hz), 7.56-7.64 (m, 1.5H), 8.20-8.24 (m, 1H), 8.41 (brs, 2H), 8.79 (s, 1H); MS (ESI) m/z=507.9 (MH⁺).

### Example 336

### [3-Bromo-6-(1H-pyrazol-4-yl)-8-trifluoromethyl-imidazo[1,2-a]pyridin-2-yl]-(3-furan-3-yl-2,5-dihydro-pyrrol-1-yl)-methanone (Compound 436)

Under standard HATU coupling conditions, 3-bromo-6-(1H-pyrazol-4-yl)-8-trifluoromethyl-imidazo[1,2-a]pyridine-2-carboxylic acid, and 3-furan-3-yl-2,5-dihydro-1H-pyrrole (Example 329, Step 1) gave [3-bromo-6-(1H-pyrazol-4-yl)-8-trifluoromethyl-imidazo[1,2-a]pyridin-2-yl]-(3-furan-3-yl-2,5-dihydro-pyrrol-1-yl)-methanone. ¹H NMR (d₆-DMSO, 300 MHz) [symbol] 4.47 (m, 1H), 4.56 (m, 1H), 4.72 (m, 1H), 4.76 (m, 1H), 6.16 (t, 0.5H, J=1.8 Hz), 6.20 (t, 0.5H, J=1.8 Hz), 6.79 (dd, 0.5H, J=0.9, 1.8 Hz), 6.82 (dd, 0.5H, J=0.9, 1.8 Hz), 7.59 (s, 0.5H), 7.70 (t, 0.5H, J=1.7 Hz), 7.71 (t, 0.5H, J=1.7 Hz), 7.92 (s, 0.5H), 8.20-8.24 (m, 1H), 8.40 (s, 2H), 8.78 (s, 1H); MS (ESI) m/z=492 (MH⁺).

### Example 337

### [3-Bromo-6-(1H-pyrazol-4-yl)-8-trifluoromethyl-imidazo[1,2-a]pyridin-2-yl]-[3-(3-fluoro-phenyl)-2,5-dihydro-pyrrol-1-yl]-methanone (Compound 437)

Under standard HATU coupling conditions, 3-bromo-6-(1H-pyrazol-4-yl)-8-trifluoromethyl-imidazo[1,2-a]pyridine-2-carboxylic acid, and 3-(3-fluoro-phenyl)-2,5-dihydro-1H-pyrrole (Example 326, Step 1) gave [3-bromo-6-(1H-pyrazol-4-yl)-8-trifluoromethyl-imidazo[1,2a]pyridin-2-yl]-[3-(3-fluoro-phenyl)-2,5-dihydro-pyrrol-1-yl]-methanone. ¹H NMR (d₆-DMSO, 300 MHz) [symbol] 4.55 (m, 1H), 4.74 (m, 1H), 4.82 (m, 1H), 4.99 (m, 1H), 6.57-6.65 (m, 1H), 7.12-7.50 (m, 4H), 8.25 (m, 1H), 8.41 (brs, 2H), 8.79 (s, 1H); MS (ESI) m/z=520 (MH⁺).

### Example 338

### [3-Bromo-6-(1H-pyrazol-4-yl)-8-trifluoromethyl-imidazo[1,2-a]pyridin-2-yl]-[3-(4-fluoro-phenyl)-2,5-dihydro-pyrrol-1-yl]-methanone (Compound 438)

Under standard HATU coupling conditions, 3-bromo-6-(1H-pyrazol-4-yl)-8-trifluoromethyl-imidazo[1,2-a]pyridine-2-carboxylic acid, and 3-(4-fluoro-phenyl)-2,5-dihydro-1H-pyrrole (Example 327, Step 1) gave [3-bromo-6-(1H-pyrazol-4-yl)-8-trifluoromethyl-imidazo[1,2-a]pyridin-2-yl]-(3-(4-fluoro-phenyl)-2,5-dihydro-pyrrol-1-yl]-methanone. ¹H NMR (d₆-DMSO, 300 MHz) [symbol] 4.53 (m, 1H), 4.73 (m, 1H), 4.81 (m, 1H), 4.98 (m, 1H), 6.44-6.50 (m, 1H), 7.20-7.64 (m, 4H), 8.20-8.25 (m, 1H), 8.41 (brs, 2H), 8.79 (s, 1H); MS (ESI) m/z=520 (MH⁺).

### Example 339

### [3-Chloro-6-(1H-pyrazol-4-yl)-8-trifluoromethyl-imidazo[1,2-a]pyridin-2-yl]-(3-thiazol-2-yl-2,5-dihydro-pyrrol-1-yl)-methanone (Compound 439)

Under standard HATU coupling conditions, 3-chloro-6-(1H-pyrazol-4-yl)-8-trifluoromethyl-imidazo[1,2-a]pyridine-2-carboxylic acid, and 2-(2,5-dihydro-1H-pyrrol-3-yl)-thiazole (Example 328, Step 1,) gave [3-chloro-6-(1H-pyrazol-4-yl)-8-trifluoromethyl-imidazo[1,2-a]pyridin-2-yl]-(3-thiazol-2-yl-2,5-dihydro-pyrrol-1-yl)-methanone. ¹H NMR (d₆-DMSO, 300 MHz) [symbol] 4.58 (m, 1H), 4.77 (m, 1H), 4.93 (m, 1H), 5.12 (m, 1H), 6.68-6.74 (m, 1H), 7.76 (d, 0.5H, J=3.2 Hz), 7.79 (d, 0.5H, J=3.2 Hz), 7.85 (d, 0.5H, J=3.2 Hz), 7.90 (d, 0.5H, J=3.2 Hz), 8.23 (s, 1H), 8.26 (s, 1H), 8.57 (s, 1H), 8.86 (s, 1H), 13.16 (brs, 1H); MS (ESI) m/z=465 (MH⁺).

### Example 340

### [6-(1H-Pyrazol-4-yl)-8-trifluoromethyl-imidazo[1,2-a]pyridin-2-yl]-(3-thiazol-2-yl-2,5-dihydro-pyrrol-1-yl)-methanone (Compound 440)

Under standard HATU coupling conditions, 6-(1H-pyrazol-4-yl)-8-trifluoromethyl-imidazo[1,2-a]pyridine-2-carboxylic acid, and 2-(2,5-dihydro-1H-pyrrol-3-yl)-thiazole (Example 328, Step 1,) gave [6-(1H-pyrazol-4-yl)-8-trifluoromethyl-imidazo[1,2-a]pyridin-2-yl]-(3-thiazol-2-yl-2,5-dihydro-pyrrol-1-yl)-methanone. ¹H NMR (d₆-DMSO, 300 MHz) [symbol] 4.57 (m, 1H), 4.77 (m, 1H), 5.07 (m, 1H), 5.26 (m, 1H), 6.68-6.78 (m, 1H), 7.78 (d, 0.5H, J=3.2 Hz), 7.79 (d, 0.5H, J=3.2 Hz), 7.88 (d, 0.5H, J=3.2 Hz), 7.89 (d, 0.5H, J=3.2 Hz), 8.04 (s, 1H), 8.11 (s, 1H), 8.41 (s, 1H), 8.50 (s, 1H), 9.16 (s, 1H), 13.13 (brs, 1H); MS (ESI) m/z=431 (MH⁺).

### Example 341

### [3-Chloro-6-(1H-pyrazol-4-yl)-8-trifluoromethyl-imidazo[1,2-a]pyridin-2-yl]-(3-thiophen-2-yl-pyrrolidin-1-yl)-methanone (Compound 441)

Under standard HATU coupling conditions, 3-chloro-6-(1H-pyrazol-4-yl)-8-trifluoromethyl-imidazo[1,2-a]pyridine-2-carboxylic acid, and 3-thiophen-2-yl-pyrrolidine (Example 323, Step 1) gave [3-chloro-6-(1H-pyrazol-4-yl)-8-trifluoromethyl-imidazo[1,2-a]pyridin-2-yl]-(3-thiophen-2-yl-pyrrolidin-1-yl)-methanone. ¹H NMR (d₆-DMSO, 300 MHz) [symbol] 1.96 (m, 1H), 2.30-2.45 (m, 1H), 3.46-4.34 (m, 5H), 6.94-7.05 (m, 2H), 7.38 (dd, 0.5H, J=1.8, 4.4 Hz), 7.41 (dd, 0.5H, J=1.5, 5 Hz), 8.19 (s, 0.5H), 8.20 (s, 0.5H), 8.24 (s, 1H), 8.56 (s, 1H), 8.82 (s, 0.5H), 8.83 (s, 0.5H), 13.16 (s, 1H); MS (ESI) m/z=466 (MH⁺).

### Example 342

### [3-Bromo-6-(1H-pyrazol-4-yl)-8-trifluoromethyl-imidazo[1,2-a]pyridine-2-yl]-(3-thiophen-2-yl-2.5-dihydro-pyrrol-1-yl)-methanone (Compound 442)

Prepared using experimental procedure described in Example 322 (compound 422). ¹H NMR (d₆-DMSO, 300 MHz) [symbol] 8.76 (s, 1H), 8.39 (s, 1H), 8.20 (br s, 1H), 7.52 (t, 1H, J=3 Hz), 7.19 (d, 1H, J=6 Hz), 7.06 (m, 1H), 6.99 (d, 1H, J=7 Hz), 6.19 (d, 1H, J=8 Hz), 4.98 (br s, 1H), 4.75 (br s, 1H), 4.68 (br s, 1H), 4.48 (br s, 1H); MS (ESI) m/z 508 (MH⁺).

### Example 343

### [3-Chloro-6-(1H-pyrozol-4-yl)-8-(trifluoromethyl)imidazo[1,2-a]pyridine-2-yl][3-(thiophen-2-yl)-2,5-dihydro-1H-pyrrol-1-yl]methanone (Compound 443)

Prepared using experimental procedure described in Example 322 (compound 422). ¹H NMR (d₆-DMSO, 300 MHz) [symbol] 8.84 s, 1H), 8.40 (d, 2H, J=2 Hz), 8.20 (m, 1H), 7.52 (m, 1H), 7.19 (d, 1H, J=3 Hz), 7.07 (m, 1H), 6.99 (m, 1H), 6.19 (m, 1H), 5.02 (br s, 1H), 4.81 (br s, 1H), 4.68 (br s, 1H), 4.48 (br s, 1H); MS (ESI) m/z 464 (MH⁺).

### Example 344

### [3-Chloro-6-(1H-pyrazol-4-yl)-8-(trifluoromethyl)imidazo[1,2-a]pyridin-2-yl][3-(furan-2-yl)-2,5-dihydro-1H-pyrrol-1-yl]methanone (Compound 444)

### Step 1: 3-Furan-2-yl-2,5-dihydro-pyrrole-1-carboxylic Acid Tert-butyl Ester

3-Trifluoromethanesulfonyloxy-2,5-dihydro-pyrrole-1-carboxylic acid tert-butyl ester, (0.12 g 0.38 mmol) was combined with 2-(tributylstannyl) furan (0.36 mL, 1.1 mmol) in THF (3 mL). To this solution was added Pd(PPh₃)₄ (43.9 mg, 0.036 mmol) and reaction mixture was stirred at 60° C. for 45 minutes. All the solids were filtered out and the resulting filtrate was concentrated to yield crude product. The crude was purified using silica gel chromatography [n-hexane/EtOAc (10:1 v/v)] to give 3-furan-2-yl-2,5-dihydro-pyrrole-1-carboxylic acid tert-butyl ester (0.042 g, 47.2%). MS (ESI) m/z 236 (MH⁺).

### Step 2: 3-Furan-2-yl-2,5-dihydro-1H-pyrrole,

3-Furan-2-yl-2,5-dihydro-pyrrole-1-carboxylic acid tert-butyl ester (42 mg) was stirred in CH₂Cl₂/TFA (3:1 v/v, 4 mL) at room temperature. After 1 hour, the mixture was evaporated to dryness. The material was used without further purification in the next step as a TFA salt. MS (ESI) m/z 218 (MH⁺).

### Step 3: [3-Chloro-6-(1H-pyrazol-4-yl)-8-trifluoromethyl-imidazo[1,2-a]pyridine-2-yl]-(3-furan-2-yl-2,5-dihydro-pyrrol-1-yl)-methanone (compound 444)

3-Chloro-6-(1H-pyrazol-4-yl)-8-trifluoromethyl-imidazo[1,2-a]pyridine-2-carboxylic acid (295 mg, 0.89 mmol) was combined with 3-furan-2-yl-2.5-dihydro-1H-pyrrole TFA salt, (252 mg, 0.89 mmol) in DMF (4 mL). To this suspension was added HATU (340 mg, 0.89 mmol) followed by N,N-diisopropylethylamine (0.8 mL, 4.5 mmol). Reaction mixture was stirred at room temperature for 30 minutes. It was diluted with EtOAc, and extracted using saturated aqueous NaHCO₃. The organic phase was separated, washed with H₂O, dried (MgSO₄), filtered and concentrated. The crude was purified using reverse phase HPLC. ¹H NMR (d₆-DMSO, 300 MHz) [symbol] 8.84s, 1H), 8.20 (br s, 1H), 7.71 (m, 1H), 6.60 (d, 0.5H, J=3 Hz), 6.53 (m, 1H), 6.33 (d, 0.5H, J=3 Hz), 6.18 (br s, 1H), 4.92 (br s, 1H), 4.83 (br s, 1H), 4.60 (br s, 1H), 4.50 (br s, 1H); MS (ESI) m/z 448 (MH⁺).

### Example 345

### [3-Chloro-6-(1H-pyrazol-4-yl)-8-(trifluoromethyl)imidazol[1,2-a]pyridine-2-yl][3-(1,3-thiazol-4-yl)-2,5-dihydro-1H-pyrrol-1-yl]methanone (Compound 445)

Prepared using experimental procedure described in Example 344 (compound 444). ¹H NMR (d₆-DMSO, 300 MHz) [symbol] 9.15 (br s, 1H), 9.11 (br s, 0.5H), 8.84 (s, 1H), 8.40 (s, 2H), 8.21 (br s, 1H), 7.82 (d, 1H, J=1 Hz), 7.55 (d, 0.5H, J=2 Hz), 6.49 (br s, 1H), 4.98 (br s, 1H), 4.85 (br s, 1H), 4.71 (br s, 1H), 4.52 (br s, 1H); MS (ESI) m/z 465 (MH⁺).

### Example 346

### (3-Bromo-6-furan-3-yl-8-trifluoromethyl-imidazo[1,2-a]pyridin-2-yl)-(3-(3-fluoro-phenyl)-pyrrolidin-1-yl)-methanone (Compound 446)

Using similar method as for the preparation of (3-bromo-6-furan-3-yl-8-trifluoromethyl-imidazo[1,2-a]pyridin-2-yl)-[3-(4-fluoro-phenyl)-pyrrolidin-1-yl]-methanone (Example 153, compound 253), 3,6-dibromo-8-trifluoromethyl-imidazo[1,2-a]pyridine-2-carboxylic acid was coupled to 3-(3-fluorophenyl)pyrrolidine followed by Suzuki reaction with furan-3-boronic acid to give (3-bromo-6-furan-3-yl-8-trifluoromethyl-imidazo[1,2-a]pyridin-2-yl)-(3-(3-fluoro-phenyl)-pyrrolidin-1-yl)-methanone. ¹H NMR (d₆-DMSO, 300 MHz) [symbol] 2.00-2.36 (m, 2H), 3.40-4.10 (m, 4.5H), 4.19 (dd, 0.5H, J=7.6, 11.1 Hz), 7.00-7.42 (m, 5H), 7.82 (t, 0.5H, J=1.8 Hz), 7.83 (t, 0.5H), J=1.8 Hz), 8.16 (s, 0.5H), 8.19 (s, 0.5H), 8.53 (t, 0.5H, J=1.2 Hz), 8.54 (t, 0.5H, J=1.2 Hz), 8.72 (s, 0.5H), 8.74 (s, 0.5H); MS (ESI) m/z=522 (MH⁺).

### Example 347

### (3-Bromo-6-(1H-pyrazol-4-yl)-8-trifluoromethyl-imidazo[1,2-a]pyridin-2-yl)-(3-(3-fluoro-phenyl)-pyrrolidin-1-yl)-methanone (Compound 447)

Prepared similarly to ((3-bromo-6-furan-3-yl-8-trifluoromethyl-imidazo[1,2-a]pyridin-2-yl)-(3-(3-fluoro-phenyl)-pyrrolidin-1-yl)-methanone (compound 446) with the use of 4-pyrazoleboronic acid pinacol ester for the Suzuki reaction. ¹H NMR (d₆-DMSO, 300 MHz) [symbol] 2.00-2.40 (m, 2H), 3.40-4.10 (m, 4.5H), 4.20 (dd, 0.5H, J=7.3, 10.8 Hz), 7.00-7.42 (m, 5H), 8.16 (s, 0.5H), 8.19 (s, 0.5H), 8.22 (s, 1H), 8.53 (s, 1H), 8.73 (s, 0.5H), 8.75 (s, 0.5H), 13.14 (brs, 1H); MS (ESI) m/z=524.1 (MH⁺).

### Example 348

### ((3-Bromo-6-furan-3-yl-8-trifluoromethyl-imidaz[1,2-a]pyridin-2-yl)-(3-(2-fluoro-phenyl)-pyrrolidin-1-yl)-methanone (448)

Using similar method as for the preparation of (3-bromo-6-furan-3-yl-8-trifluoromethyl-imidazo[1,2-a]pyridin-2-yl)-[3-(4-fluoro-phenyl)-pyrrolidin-1-yl]-methanone (Example 153, compound 253), 3,6-dibromo-8-trifluoromethyl-imidazo[1,2-a]pyridine-2-carboxylic acid was coupled to 3-(2-fluorophenyl)pyrrolidine followed by Suzuki reaction with furan-3-boronic acid to give ((3-bromo-6-furan-3-yl-8-trifluoromethyl-imidazo[1,2-a]pyridin-2-yl)-(3-2-fluoro-phenyl)-pyrrolidin-1-yl)-methanone. ¹H NMR (d₆-DMSO, 300 MHz) [symbol] 2.04-2.36 (m, 2H), 3.48-4.08 (m, 4.5H), 4.22 (dd, 0.5H, J=6.5, 10.3 Hz), 7.12-7.46 (m, 5H), 7.82 (t, 0.5H, J=1.8 Hz), 7.83 (t, 0.5H), J==1.8 Hz), 8.16 (s, 0.5H), 8.19 (s, 0.5H), 8.52 (s, 0.5H), 8.54 (s, 0.5H), 8.72 (s, 0.5H), 8.74 (s, 0.5H); MS (ESI) m/z=524 (MH⁺).

### Example 349

### (3-Bromo-6-(1H-pyrazol-4-yl)-8-trifluoromethyl-imidazo[1,2-a]pyridin-2-yl)-(3-(2-fluoro-phenyl)-pyrrolidin-1-yl)-methanone (Compound 449)

Prepared similar to (3-bromo-6-furan-3-yl-8-trifluoromethyl-imidazo[1,2-a]pyridin-2-yl)-(3-(2-fluoro-phenyl)-pyrrolidin-1-yl)-methanone (compound 448) with the use of 4-pyrazoleboronic acid pinacol ester for the Suzuki reaction. ¹H NMR (d₆-DMSO, 300 MHz) [symbol] 2.04-2.36 (m, 2H), 3.48-4.08 (m, 4.5H), 4.23 (dd, 0.5H, J=6.7, 11.4 Hz), 7.10-7.46 (m, 4H), 8.16 (s, 0.5H), 8.19 (s, 0.5H), 8.22 (brs, 1H), 8.53 (brs, 1H), 8.73 (s, 0.5H), 8.75 (s, 0.5H), 13.12 (brs, 1H); MS (ESI) m/z=524 (MH⁺).

### Example 350

### 3-(1-(3-Chloro-6-furan-3-yl-8-trifluoro methyl-imidazo[1,2-a]pyridine-2-carbonyl)-pyrrolidin-3-yl)-benzonitrile (Compound 450)

Prepared using standard HATU coupling. ¹H NMR (d₆-DMSO, 300 MHz) [symbol] 2.23-2.40 (m, 2H), 3.42-4.13 (m, 4.5H), 4.27 (dd, 0.5H, J=7, 11.1 Hz), 7.30 (m, 1H), 7.54 (q, 1H, J=7.9 Hz), 7.65-7.87 (m, 4H), 8.17 (s, 0.5H), 8.19 (s, 0.5H), 8.53 (s, 0.5H), 8.55 (s, 0.5H), 8.80 (s, 0.5H), 8.82 (s, 0.5H); MS (ESI) m/z=485.1 (MH⁺).

### Example 351

### (3-Chloro-6-furan-3-yl-8-trifluoromethyl-imidaz [1,2-a]pyridin-2-yl)-[3-(3-methoxy-phenyl)-pyrrolidin-1-yl]-methanone (Compound 451)

Prepared using standard HATU coupling. ¹H NMR (d₆-DMSO, 300 MHz) [symbol] 1.98-2.36 (m, 2H), 3.40-4.12 (m, 4.5H), 3.73 (s, 1.5H), 3.75 (s, 1.5H), 4.26 (dd, 0.5H, J=7, 10.8 Hz), 6.76-6.94 (m, 3H), 7.24 (q, 1H, J=8.2 Hz), 7.30 (dd, 0.5H, J=0.9, 2.0 Hz), 7.31 (dd, 0.5H, J=0.9, 2.0 Hz), 7.82 (t, 0.5H, J=1.8 Hz), 7.83 (t, 0.5H, J=1.8 Hz), 8.16 (s, 0.5H), 8.19 (s, 0.5H), 8.53 (s, 0.5H), 8.54 (s, 0.5H), 8.79 (s, 0.5H), 8.81 (s, 0.5H); MS (ESI) m/z=490.1 (MH⁺).

### Example 352

### 3-(1-(3-Chloro-6-furan-3-yl-8-trifluoromethyl-imidazo[1,2-a]pyridine-2-carbonyl)-pyrrolidin-3-yl)-benzoic acid methyl ester (Compound 452)

Prepared using standard HATU coupling. ¹H NMR (d₆-DMSO, 300 MHz) [symbol] 2.00-2.42 (m, 2H), 3.46-4.13 (m, 4.5H), 3.84 (s, 1.5H), 3.86 (s, 1.5H), 4.31 (dd, 0.5H, J=7, 11.1 Hz), 7.30 (dd, 0.5H, J=0.6, 1.8 Hz), 7.31 (dd, 0.5H, J=0.6, 1.8 Hz), 7.49 (q, 1H, J=8 Hz), 7.61 (brd, 0.5H, J=7.9 Hz), 7.67 (brd, 0.5H, J=7.9 Hz), 7.80-7.92 (m, 3H), 8.16 (s, 0.5H), 8.19 (s, 0.5H), 8.53 (s, 0.5H), 8.55 (s, 0.5H), 8.80 (s, 0.5H), 8.82 (s, 0.5H); MS (ESI) m/z=518.1 (MH⁺).

### Example 353

### (3-Chloro-6-furan-3-yl-8-trifluoromethyl-imidazo[1,2-a]pyridin-2-yl)-(3-pyridin-3-yl-pyrrolidin-1-yl)-methanone (Compound 453)

Prepared using standard HATU coupling. ¹H NMR (d₆-DMSO, 300 MHz) [symbol] 2.10-2.50 (m, 2H), 3.50-4.18 (m, 4.5H), 4.32 (dd, 0.5H, J=7, 10.8 Hz), 7.32 (m, 1H), 7.82-7.93 (m, 2H), 8.18 (s, 0.5H), 8.21 (s, 0.5H), 8.35 (brd, 0.5H, J=8.2 Hz), 8.45 (brd, 0.5H, J=8.2 Hz), 8.54 (s, 0.5H), 8.56 (s, 0.5H), 8.65-8.88 (m, 3H); MS (ESI) m/z=461.1 (MH⁺).

### Example 354

### (3-Chloro-6-furan-3-yl-8-trifluoromethyl-imidazo[1,2-a]pyridin-2-yl)-(3-pyridin-4-yl-pyrrolidin-1-yl)-methanone (Compound 454)

Prepared using standard HATU coupling. ¹H NMR (d₆-DMSO, 300 MHz) [symbol] 2.08-2.50 (m, 2H), 3.50-4.20 (m, 4.5H), 4.34 (dd, 0.5H, J=7, 10.8 Hz), 7.32 (m, 1H), 7.83 (q, 1H, J=1.7 Hz), 7.88 (s, 0.5H), 7.90 (s, 0.5H), 7.96 (s, 0.5H), 7.98 (s, 0.5H), 8.19 (s, 0.5H), 8.21 (s, 0.5H), 8.55 (s, 0.5H), 8.56 (s, 0.5H), 8.77-8.85 (m, 3H); MS (ESI) m/z=461.1 (MH⁺).

### Example 355

### 3-(1-(3-Chloro-6-furan-3-yl-8-trifluoromethyl-imidazo[1,2-a]pyridine-2-carbonyl)-pyrrolidin-3-yl)-benzoic Acid (Compound 455)

Saponification of 3-(1-(3-chloro-6-furan-3-yl-8-trifluoromethyl-imidazo[1,2-a]pyridine-2-carbonyl)-pyrrolidin-3-yl)-benzoic acid methyl ester (compound 452) using lithium [symbol] hydroxide gave 3-(1-(3-chloro-6-furan-3-yl-8-trifluoromethyl-imidazo[1,2-a]pyridine-2-carbonyl)-pyrrolidin-3-yl)-benzoic acid (compound 455) ¹H NMR (d₆-DMSO, 300 MHz) [symbol] 2.00-2.42 (m, 2H), 3.44-4.12 (m, 4.5H), 4.30 (dd, 0.5H, J=7.3, 11.1 Hz), 7.30 (dd, 0.5H, J=0.6, 1.8 Hz), 7.32 (dd, 0.5H, J=0.6, 1.8 Hz), 7.46 (q, 1H, J=7.9 Hz), 7.57 (brd, 0.5H, J=7.9 Hz), 7.63 (brd, 0.5H, J=7.9 Hz), 7.78-7.92 (m, 3H), 8.17 (s, 0.5H), 8.20 (s, 0.5H), 8.53 (s, 0.5H), 8.55 (s, 0.5H), 8.80 (s, 0.5H), 8.82 (s, 0.5H); MS (ESI) m/z=504.1 (MH⁺).

### Example 356

### (-1-(3-Chloro-6-furan-3-yl-8-trifluoromethyl-imidazo[1,2-a]pyridine-2-carbonyl)-4-phenyl-pyrrolidin-3-yl)-carbamic acid tert-butyl ester (Compound 456)

Prepared using standard HATU coupling. ¹H NMR (d₆-DMSO, 300 MHz) [symbol] 1.30 (s, 4.5H), 1.34 (s, 4.5H), 3.30-4.37 (m, 6H), 7.20-7.40 (m, 7H), 7.82 (t, 0.5H, J=1.8 Hz), 7.83 (t, 0.5H, J=1.8 Hz), 8.16 (s, 0.5H), 8.19 (s, 0.5H), 8.53 (s, 0.5H), 8.55 (s, 0.5H), 8.80 (s, 0.5H), 8.81 (s, 0.5H); MS (ESI) m/z=575.2 (MH⁺).

### Example 357

### (3-Amino-4-phenyl-pyrrolidin-1-yl)-(3-chloro-6-furan-3-yl-8-trifluoromethyl-imidazo[1,2-a]pyridin-2-yl)-methanone (Compound 457)

To a solution of (1-(3-chloro-6-furan-3-yl-8-trifluoromethyl-imidazo[1,2-a]pyridine-2-carbonyl)-4-phenyl-pyrrolidin-3-yl)-carbamic acid tert-butyl ester (333 mg, 0.5791 mmol) in CH₂Cl₂ (10 mL) was added 2M HCl in Et₂O (5 mL). After 2.5 hours, 2M HCl in Et₂O (5 mL) was added and the mixture was stirred overnight. The white precipitate was filtered and dried under high vacuum to give 3-amino-4-phenyl-pyrrolidin-1-yl)-(3-chloro-6-furan-3-yl-8-trifluoromethyl-imidazo[1,2-a]pyridin-2-yl)-methanone (285 mg, 96%) as a white powder. ¹H NMR (d₆-DMSO, 300 MHz) [symbol] 3.58-4.48 (m, 6H), 7.30-7.44 (m, 6H), 7.83 (t, 0.5H, J=1.8 Hz), 7.84 (t, 0.5H, J=1.8 Hz), 8.18 (s, 0.5H), 8.23 (s, 0.5H), 8.42 (brs, 3H), 8.54 (s, 0.5H), 8.57 (s, 0.5H), 8.81 (s, 0.5H), 8.85 (s, 0.5H); MS (ESI) m/z=475.1 (MH⁺).

### Example 358

### N-(-1-(3-Chloro-6-furan-3-yl-8-trifluoromethyl-imidazo[1,2-a]pyridine-2-carbonyl)-4-phenyl-pyrrolidin-3-yl)-methanesulfonamide (Compound 458)

To a solution of (3-amino-4-phenyl-pyrrolidin-1-yl)-(3-chloro-6-furan-3-yl-8-trifluoromethyl-imidazo[1,2-a]pyridin-2-yl)-methanone (50 mg, 0.09778 mmol) in DMF (1 mL) was added N,N-diisopropylethylamine (85 μL, 0.4889 mmol), and methanesulfonyl chloride (11.4 μL, 0.1467 mmol). After 1 hour, the mixture was diluted with EtOAc (20 mL), and washed with saturated aqueous NaHCO₃ (10 mL), then brine (10 mL). The extracts were dried (Na₂SO₄), filtered and concentrated. Preparative HPLC purification (30-100% ACN gradient) of the crude material gave N-(-1-(3-cloro-6-furan-3-yl-8-trifluoromethyl-imidazo[1,2-a]pyridine-2-carbonyl)-4-phenyl-pyrrolidin-3-yl)-methanesulfonamide (compound 458) (33.8 mg, 63%) as a white powder. ¹H NMR (d₆-DMSO, 300 MHz) [symbol] 2.47 (s, 1.5H), 2.62 (s, 1.5H), 3.32 (m, 0.5H), 3.60 (t, 0.5H, J=11 Hz), 3.73 (dd, 0.5H, J=9.1, 11.4 Hz), 4.02-4.24 (m, 2H), 4.32 (dd, 0.5H, J=7.6, 11.4 Hz), 4.39 (dd, 0.5H, J=7.6, 11.4 Hz), 7.24-7.46 (m, 6H), 7.65 (d, 0.5H, J=8.2 Hz), 7.69 (d, 0.5H, J=8.5 Hz), 7.82 (t, 0.5H, J=1.8 Hz), 7.83 (t, 0.5H, J=1.8 Hz), 8.17 (s, 0.5H), 8.21 (s, 0.5H), 8.53 (s, 0.5H), 8.56 (s, 0.5H), 8.80 (s, 0.5H), 8.82 (s, 0.5H); MS (ESI) m/z=553.1 (MH⁺).

### Example 359

### N-(-1-(3-Chloro-6-furan-3-yl-8-trifluoromethyl-imidazo[1,2-a]pyridine-2-carbonyl)-4-phenyl-pyrrolidin-3-yl)-acetamide (Compound 459)

To a solution of (3-amino-4-phenyl-pyrrolidin-1-yl)-(3-chloro-6-furan-3-yl-8-trifluoromethyl-imidazo[1,2-a]pyridin-2-yl)-methanone (50 mg, 0.09778 mmol) in DMF (1 mL) was added N,N-diisopropylethylamine (85 μL, 0.4889 mmol), and acetic anhydride (13.9 μL, 0.1467 mmol). After 1 hour, the mixture was diluted with EtOAc (20 mL), and washed with saturated aqueous NaHCO₃ (10 mL), then brine (10 mL). The extracts were dried (Na₂SO₄), filtered and concentrated. Preparative HPLC purification (30-100% ACN gradient) of the crude material gave N-(-1-(3-chloro-6-furan-3-yl-8-trifluoromethyl-imidazo[1,2-a]pyridine-2-carbonyl)-4-phenyl-pyrrolidin-3-yl)-acetamide (compound 459) (40.8 mg, 90%) as a white powder. ¹H NMR (d₆-DMSO, 300 MHz) [symbol] 1.74 (s, 1.5H), 1.80 (s, 1.5H), 3.30-4.60 (m, 6H), 7.20-7.40 (m, 6H), 7.82 (t, 0.5H, J=1.5 Hz), 7.83 (t, 0.5H, J=1.8 Hz), 8.17-8.26 (m, 2H), 8.54 (s, 0.5H), 8.55 (s, 0.5H), 8.81 (s, 1H); MS (ESI) m/z=517.1 (MH⁺).

### Example 360

### (3-Chloro-6-furan-3-yl-8-trifluoromethyl-imidazo[1,2-a]pyridin-2-yl)-[3-(3-chloro-phenyl)-pyrrolidin-1-yl]-methanone (Compound 460)

Prepared using standard HATU coupling. ¹H NMR (d₆-DMSO, 300 MHz) [symbol] 1.98-2.38 (m, 2H), 3.40-4.12 (m, 4.5H), 4.26 (dd, 0.5H, J=7.6, 11.4 Hz), 7.25-7.44 (m, 5H), 7.82 (t, 0.5H, J=1.8 Hz), 7.83 (t, 0.5H, J=1.8 Hz), 8.17 (s, 0.5H), 8.19 (s, 0.5H), 8.54 (s, 0.5H), 8.55 (s, 0.5H), 8.80 (s, 0.5H), 8.81 (s, 0.5H); MS (ESI) m/z=494 (MH⁺).

### Example 361

### (3-Bromo-6-(1H-pyrazol-4-yl)-8-trifluoromethyl-imidazo[1,2-a]pyridin-2-yl)-(3-phenyl-pyrrolidin-1-yl)-methanone (Compound 461)

### Step 1: (3,6-Dibromo-8-trifluoromethyl-imidazo[1,2-a]pyridin-2-yl)-(3-phenyl-pyrrolidin-1-yl)-methanone

Under standard HATU coupling conditions, 3,6-dibromo-8-trifluoromethyl-imidazo[1,2-a]pyridine-2-carboxylic acid and 3-phenylpyrrolidine gave (3,6-dibromo-8-trifluoromethyl-imidazo[1,2-a]pyridin-2-yl)-(3-phenyl-pyrrolidin-1-yl)-methanone. ¹H NMR (d₆-DMSO, 300 MHz) [symbol] 2.00-2.36 (m, 2H), 3.40-4.06 (m, 4.5H), 4.13 (dd, 0.5H, J=7.6, 10.8 Hz), 7.18-7.36 (m, 5H), 8.04 (m, 0.5H), 8.08 (m, 0.5H), 8.88 (d, 0.5H, J=0.9 Hz), 8.90 (d, 0.5H, J=1 Hz); MS (ESI) m/z=517.9 (MH⁺).

### Step 2: (3-Bromo-6-(1H-pyrazol-4-yl)-8-trifluoromethyl-imidazo[1,2-a]pyridin-2-yl)-(3-phenyl-pyrrolidin-1-yl)-methanone (compound 461)

(3-Bromo-6-(1H-pyrazol-4-yl)-8-trifluoromethyl-imidazo[1,2-a]pyridin-2-yl)-(3-phenyl-pyrrolidin-1-yl)-methanone was prepared similar to ((3-bromo-6-furan-3-yl-8-trifluoromethyl-imidazo[1,2-a]pyridin-2-yl)-(3-(2-fluoro-phenyl)-pyrrolidin-1-yl)-methanone (compound 448) with the use of 4-pyrazoleboronic acid pinacol ester for the Suzuki reaction. ¹H NMR (d₆-DMSO, 300 MHz) [symbol] 1.98-2.36 (m, 2H), 3.40-4.08 (m, 4.5H), 4.20 (dd, 0.5H, J=7.3, 10.8 Hz), 7.18-7.36 (m, 5H), 8.18 (brd, 1H, J=9.4 Hz), 8.21 (brd, 1H, J=5.3 Hz), 8.54 (s, 0.5H), 8.55 (s, 0.5H), 8.73 (s, 0.5H), 8.76 (s, 0.5H), 13.15 (brs, 1H); MS (ESI) m/z=504 (MH⁺).

### Example 362

### [3-(3-Amino-phenyl)-pyrrolidin-1-yl]-(3-chloro-6-furan-3-yl-8-trifluoromethyl-imidaz [1,2-a]pyridin-2-yl)-methanone (Compound 462)

Prepared using standard HATU coupling. ¹H NMR (d₆-DMSO, 300 MHz) [symbol] 1.96-2.12 (m, 1H), 2.24-2.40 (m, 1H), 3.40-4.12 (m, 4.5H), 4.27 (dd, 0.5H, J=7.0, 11.4 Hz), 7.04-7.42 (m, 5H), 7.82-7.84 (m, 1H), 8.18 (s, 0.5H), 8.21 (s, 0.5H), 8.54 (s, 0.5H), 8.56 (s, 0.5H), 8.81 (s, 0.5H), 8.82 (s, 0.5H); MS (ESI) m/z=475.1 (MH⁺).

### Example 363

### (3-Chloro-6-furan-3-yl-8-trifluoromethyl-imidaz [1,2-a]pyridin-2-yl)-[3-(2-methoxy-phenyl)-pyrrolidin-1-yl]-methanone (Compound 463)

Prepared using standard HATU coupling. ¹H NMR (d₆-DMSO, 300 MHz) [symbol] 2.02-2.30 (m, 1H), 2.24-2.40 (m, 1H), 3.66-3.90 (m, 3.5H), 3.76 (s, 1.5H), 3.83 (s, 1.5H), 3.96-4.08 (m, 1H), 4.20-4.32 (0.5H), 6.80-7.25 (m, 2H), 7.18-7.27 (m, 2H), 7.30 (dd, 0.5H, J=0.8, 2 Hz), 7.32 (dd, 0.5H, J=0.8, 2 Hz), 7.82 (t, 0.5H, J=1.8 Hz), 7.83 (t, 0.5H, J=1.8 Hz), 8.16 (s, 0.5H), 8.19 (s, 0.5H), 8.53 (s, 0.5H), 8.55 (s, 0.5H), 8.79 (s, 0.5H), 8.81 (s, 0.5H); MS (ESI) m/z=490.1 (MH⁺).

### Example 364

### [3-Chloro-6-(1H-pyrazol-4-yl)-8-trifluoromethyl-imidazo[1,2-a]pyridin-2-yl]-(3-phenyl-pyrrolidin-1-yl)-methanone (Compound 464)

Prepared using standard HATU coupling. ¹H NMR (d₆-DMSO, 300 MHz) [symbol] 1.80-2.24 (m, 1H), 2.24-2.38 (m, 1H), 3.38-4.42 (m, 4.5H), 4.26 (dd, 0.5H, J=7, 11.4 Hz), 7.18-7.36 (m, 5H), 8.16 (s, 0.5H), 8.19 (s, 0.5H), 8.38 (s, 1H), 8.39 (s, 1H), 8.81 (brs, 0.5H), 8.82 (brs, 0.5H); MS (ESI) m/z=460 (MH⁺).

### Example 365

### [3-Chloro-6-(1H-pyrazol-4-yl)-8-trifluoromethyl-imidazo[1,2-a]pyridin-2-yl]-(3-(S)-phenyl-pyrrolidin-1-yl)-methanone (Compound 465)

Prepared using standard HATU coupling. ¹H NMR (d₆-DMSO, 300 MHz) [symbol] 1.98-2.14 (m, 1H), 2.24-2.36 (m, 1H), 3.40-4.12 (m, 4.5H), 4.27 (dd, 0.5H, J=7.3, 11.1 Hz), 7.18-7.38 (m, 5H), 8.16 (s, 0.5H), 8.19 (s, 0.5H), 8.23 (brs, 1H), 8.54 (brs, 1H), 8.81 (brs, 0.5H), 8.83 (brs, 0.5H), 13.14 (s, 1H); MS (ESI) m/z=460 (MH⁺).

### Example 366

### [3-Chloro-6-(1H-pyrazol-4-yl)-8-trifluoromethyl-imidazo[1,2-a]pyridin-2-yl]-(3-(R)-phenyl-pyrrolidin-1-yl)-methanone (Compound 466)

Prepared using standard HATU coupling. ¹H NMR (d₆-DMSO, 300 MHz) [symbol] 1.98-2.14 (m, 1H), 2.24-2.36 (m, 1H), 3.40-4.12 (m, 4.5H), 4.27 (dd, 0.5H, J=7.3, 11.1 Hz), 7.18-7.38 (m, 5H), 8.16 (s, 0.5H), 8.19 (s, 0.5H), 8.23 (brs, 1H), 8.54 (brs, 1H), 8.81 (brs, 0.5H), 8.83 (brs, 0.5H), 13.14 (s, 1H); MS (ESI) m/z=460 (MH⁺).

### Example 368

### (3-Chloro-6-furan-3-yl-8-trifluoromethyl-imidazo[1,2-a]pyridin-2-yl)-(3-pyridin-2-yl-pyrrolidin-1-yl)-methanone (Compound 468)

Prepared using standard HATU coupling and isolated as hydrochloride salt. ¹H NMR (d₆-DMSO, 300 MHz) [symbol] 2.20-2.48 (m, 2H), 3.60-4.16 (m, 4.5H), 4.29 (dd, 0.5H, J=7.3, 11 Hz), 7.31 (dd, 0.5H, J=0.8, 1.8 Hz), 7.32 (dd, 0.5H, J=0.8, 1.8 Hz), 7.56-7.86 (m, 3H), 8.10-8.28 (m, 2H), 8.54 (s, 0.5H), 8.56 (s, 0.5H), 8.67 (brd, 0.5H, J=4 Hz), 8.73 (brd, 0.5H, J=4.4 Hz), 8.81 (brs, 0.5H), 8.82 (brs, 0.5H); MS (ESI) m/z=461.1 (MH⁺).

### Example 372

### [5-(5-Bromo-2-hydroxy-phenyl)-3-furan-3-y-4,5-dihydro-pyrazol-1-yl]-(3-chloro-6-furan-3-yl-8-trifluoromethyl-imidazo[1,2-a]pyridin-2-yl)-methanone (Compound 472)

Under standard HATU coupling conditions, 3-chloro-6-furan-3-yl-8-trifluoromethyl-imidazo[1,2-a]pyridine-2-carboxylic acid, and 4-bromo-2-(5-furan-3-yl-3,4-dihydro

-2H-pyrazol-3-yl)-phenol gave [5-(5-bromo-2-hydroxy-phenyl)-3-furan-3-yl-4,5-dihydro-pyrazol-1-yl]-(3-chloro-6-furan-3-yl-8-trifluoromethyl-imidazo[1,2-a]pyridin-2-yl)-methanone. ¹H NMR (d₆-DMSO, 300 MHz) [symbol] 3.07 (dd, 1H, J=4.7, 17.5 Hz), 3.87 (dd, 1H, J=11.5, 17.5 Hz), 5.82 (dd, 1H, J=4.7, 11.5 Hz), 6.61 (dd, 1H, J=1.8, 3.5 Hz), 6.86 (d, 1H, J=8.5 Hz), 6.99 (d, 1H, J=3.2 Hz), 7.21 (d, 1H, J=2.3 Hz), 7.29 (dd, 1H, J=2.3, 8.5 Hz), 7.34 (d, 1H, J=1.5 Hz), 7.81 (d, 1H, J=1.2 Hz), 7.84 (t, 1H, J=1.5 Hz), 8.21 (s, 1H), 8.57 (s, 1H), 8.87 (s, 1H), 10.25 (s, 1H); MS (ESI) m/z=619 (MH⁺).

### Example 374

### 2-[3-(3-Fluoro-phenyl)-pyrroidin-1-ylmethyl]-6-furan-3-yl-8-trifluoromethyl-imidazo[1,2-a]pyridine (Compound 474)

A mixture of (3-chloro-6-furan-3-yl-8-trifluoromethyl-imidazo[1,2-a]pyridin-2-yl)-[3-(3-fluoro-phenyl)-pyrrolidin-1-yl]-methanone (Compound 334, 210 mg, 0.302 mmol) and Lawesson's reagent (122 mg, 0.302 mmol) was heated in THF (2.5 mL) for 1.5 hr. The solvent was concentrated under vacuo and the crude material chromatographed [n-hex/EtOAc (5:1 v/v)] to give (3-chloro-6-furan-3-yl-8-trifluoromethyl-imidazo[1,2-a]pyridin-2-yl)-[3-(3-fluoro-phenyl)-pyrrolidin-1-yl]-methanethione (110 mg, 74%) as a yellow solid.

To a solution of the above intermediate (64 mg, 0.130 mmol) and nickel(II) chloride hexahydrate (77 mg, 0.324 mmol) in THF (7 mL) and MeOH (7 mL) at 0° C. was added sodium borohydride (36.8 mg, 0.972 mmol) in one portion. After 20 min, black precipitate was filtered and washed with MeOH. The filtrate was concentrated and the crude material was chromatographed [CHCl₃/MeOH (95:5 v/v)] to give 2-[3-(3-fluoro-phenyl)-pyrrolidin-1-ylmethyl]-6-furan-3-yl-8-trifluoromethyl-imidazo[1,2-a]pyridine which was converted to the HCl salt (44.5 mg, 80%) isolated as a white powder. ¹H NMR (d₆-DMSO, 300 MHz) δ 1.93-2.48 (m, 2H), 3.30-3.86 (m, 5H), 4.64 (s, 2H), 7.04-7.42 (m, 5H), 7.82 (t, 1H, J=1.8 Hz), 8.08 (s, 1H), 8.31 (s, 0.5H), 8.33 (s, 0.5H), 8.43 (s, 1H), 9.27 (s, 1H), 11.45 (brs, 0.5H), 11.64 (brs, 0.5H); MS (ESI) m/z=430.1 (MH⁺).

### Example 375

### (3-Chloro-6-furan-3-yl-8-trifluoromethyl-imidaz[1,2-a]pyridin-2-yl)-[3-(3-methyl-[1,2,4]oxadiazol-5-yl)-pyrrolidin-1-yl]-methanone (Compound 475)

### Step 1: 3-Methyl-5-pyrrolidin-3-yl-[1,2,4]oxadiazole Hydrochloride

To a solution of 1-Boc-pyrrolidine-3-carboxylic acid (215.3 mg, 1 mmol) in DMF (5 mL) was added N,N-diisopropylethylamine (0.61 mL, 3.5 mmol), HATU (380.2 mg, 1 mmol) and N-hydroxyacetamidine (81.5 mg, 1.1 mmol). After 3 hours, the mixture was diluted with DMF (15 mL) and the mixture was subjected to heating at 120° C. under microwave conditions for 30 min. The solvent was concentrated and diluted with EtOAc (50 mL) and washed with saturated aqueous NaHCO₃ (25 mL), then brine (25 mL). The filtrate was diluted with n-hex (50 mL), passed through a short pad of silica gel, and washed with n-hex/EtOAc (1:1 v/v). The solvents was concentrated to give 3-(3-methyl-[1,2,4]oxadiazol-5-yl)-pyrrolidine-1-carboxylic acid tert-butyl ester as a yellow oil (192 mg). To a solution of the above compound in CH₂Cl₂ (4 mL) was added 4M HCl in dioxane (3 mL). After 1.5 hours, the solvent was concentrated under vacuo to give 3-methyl-5-pyrrolidin-3-yl-[1,2,4]oxadiazole hydrochloride (149 mg) as a beige solid. ¹H NMR (d₆-DMSO, 300 MHz) [symbol] 2.10-2.25 (m, 1H), 2.34 (s, 3H), 2.32-2.50 (m, 1H), 3.20-3.35 (m, 2H), 3.43 (dd, 1H, J=7, 11.7 Hz), (dd, 1H, J=8.2, 11.7 Hz), 3.92 (p, 1H, J=7.9 Hz), 9.35 (brs, 2H).

### Step 2: (3-Chloro-6-furan-3-yl-8-trifluoromethyl-imidaz[1,2-a]pyridin-2-yl)-[3-(3-methyl-[1,2,4]oxadiazol-5-yl)-pyrrolidin-1-yl]-methanone (Compound 475)

Under standard HATU coupling conditions, 3-chloro-6-furan-3-yl-8-trifluoromethyl-imidazo[1,2-a]pyridine-2-carboxylic acid, and 3-methyl-5-pyrrolidin-3-yl-[1,2,4]oxadiazole hydrochloride (prepared as shown in step 1) gave (3-chloro-6-furan-3-yl-8-trifluoromethyl-imidazo[1,2-a]pyridin-2-yl)-[3-(3-methyl-[1,2,4]oxadiazol-5-yl)-pyrrolidin-1-yl]-methanone. ¹H NMR (d₆-DMSO, 300 MHz) [symbol] 2.18-2.28 (m, 1H), 2.31 (s, 1.5H), 2.34 (s, 1.5H), 2.35-2.48 (m, 1H), 3.60-4.14 (m, 4.5H), 4.31 (dd, 0.5H, J=7.3, 11.4 Hz), 7.32 (d, 1H, J=1.8 Hz), 7.83 (t, 1H, J=1.8 Hz), 8.20 (s, 1H), 8.55 (s, 1H), 8.81 (s, 1H); MS (ESI) m/z=466.1 (MH⁺).

### Example 376

### [3-Chloro-6-(1H-pyrazol-4-yl)-8-trifluoromethyl-imidazo[1,2-a]pyridin-2-yl]-[3-(3-methyl-[1,2,4]oxadiazol-5-yl)-pyrrolidin-1-yl]-methanone (Compound 476)

Under standard HATU coupling conditions, 3-chloro-6-(1H-pyrazol-4-yl)-8-trifluoromethyl-imidazo[1,2-a]pyridine-2-carboxylic acid, and 3-methyl-5-pyrrolidin-3-yl-[1,2,4]oxadiazole hydrochloride gave [3-chloro-6-(1H-pyrazol-4-yl)-8-trifluoromethyl-imidazo[1,2-a]pyridin-2-yl]-[3-(3-methyl-[1,2,4]oxadiazol-5-yl)-pyrrolidin-1-yl]-methanone. ¹H NMR (d₆-DMSO, 300 MHz) δ 2.18-2.30 (m, 1H), 2.31 (s, 1.5H), 2.34 (s, 1.5H), 2.37-2.47 (m, 1H), 3.64-4.14 (m, 4.5H), 4.31 (dd, 0.5H, J=7.3, 11.7 Hz), 8.19 (s, 1H), 8.24 (s, 1H), 8.55 (s, 1H), 8.82 (s, 1H), 13.15 (s, 1H); MS (ESI) m/z=466.1 (MH⁺).

### Example 377

### (3-Chloro-6-furan-3-yl-8-trifluoromethyl-imidazo[1,2-a]pyridin-2-yl)-(3-phenyl-4,5-dihydro-pyrazol-1-yl)-methanone (Compound 477)

### Step 1: 3-Phenyl-4,5-dihydro-1H-pyrazole

To a solution of hydrazine monohydrate (1.24 mL, 25.6 mmol) in MeOH (45 mL) was added a solution of 3-chloropropiophenone (1.08 g, 6.4 mmol) in MeOH (20 mL) over 10 min. After 6 days, the solvent was concentrated and the crude material purified by RP-HPLC (0-60% ACN gradient) to give 3-phenyl-4,5-dihydro-1H-pyrazole (405 mg) as a yellow solid. ¹H NMR (d₆-DMSO, 300 MHz) [symbol] 3.42-3.50 (m, 2H), 3.58-3.66 (m, 2H), 7.49-7.62 (m, 3H), 7.82-7.86 (m, 2H); MS (ESI) m/z=147.1 (MH⁺).

### Step 2: (3-Chloro-6-furan-3-yl-8-trifluoromethyl-imidazo[1,2-a]pyridin-2-yl)-(3-phenyl-4,5-dihydro-pyrazol-1-yl)-methanone (Compound 477)

Under standard HATU coupling conditions, 3-chloro-6-furan-3-yl-8-trifluoromethyl-imidazo[1,2-a]pyridine-2-carboxylic acid, and 3-phenyl-4,5-dihydro-1H-pyrazole (prepared as shown in step 1) gave (3-chloro-6-furan-3-yl-8-trifluoromethyl-imidazo[1,2-a]pyridin-2-yl)-(3- [symbol] phenyl-4,5-dihydro-pyrazol-1-yl)-methanone. ¹H NMR (d₆-DMSO, 300 MHz) [symbol] 3.41 (t, 2H, J=9.5 Hz), 4.18 (t, 2H, J=9.5 Hz), 7.33 (d, 0.5H, J=0.8 Hz), 7.34 (d, 0.5H, J=0.6 Hz), 7.38-7.50 (m, 3H), 7.68-7.73 (m, 2H), 7.85 (t, 1H, J=1.7 Hz), 8.21 (s, 1H), 8.57 (s, 1H), 8.66 (s, 1H); MS (ESI) m/z=459 (MH⁺).

### Example 378

### [1-(3-Chloro-6-furan-3-yl-8-trifluoromethyl-imidazo[1,2-a]pyridine-2-carbonyl)-pyrrolidin-3-yl]-carbamic acid tert-butyl ester (Compound 478)

Prepared using standard HATU coupling. ¹H NMR (d₆-DMSO, 300 MHz) [symbol] 1.36 (s, 4.5H), 1.41 (s, 4.5H), 2.00-2.12 (m, 1H), 1.76-1.90 (m, 1H), 3.36-4.10 (m, 5H), 7.24 (m, 1H), 7.32 (m, 1H), 7.84 (t, 1H, J=1.7 Hz), 8.20 (s, 1H), 8.55 (s, 1H), 8.81 (s, 1H); MS (ESI) m/z=499.1 (MH⁺).

### Example 379

### (3-Amino-pyrrolidin-1-yl)-(3-chloro-6-furan-3-yl-8-trifluoromethyl-imidazo[1,2-a]pyridin-2-yl)-methanone (Compound 479)

To a solution of [1-(3-chloro-6-furan-3-yl-8-trifluoromethyl-imidazo[1,2-a]pyridine-2-carbonyl)-pyrrolidin-3-yl]-carbamic acid tert-butyl ester (0.27 g, 0.541 mmol) in CH₂Cl₂ (15 mL) was added 4M HCl in dioxane (5 mL). After 4 hours, the precipitate was filtered and dried under high vacuum to give (3-amino-pyrrolidin-1-yl)-(3-chloro-6-furan-3-yl-8-trifluoromethyl-imidazo[1,2-a]pyridin-2-yl)-methanone hydrochloride (210 mg, 89%) as a light yellow powder. ¹H NMR (d₆-DMSO, 300 MHz) [symbol] 2.18-2.32 (m, 1H), 1.94-2.12 (m, 1H), 3.60-4.21 (m, 5H), 7.33 (m, 1H), 7.85 (t, 1H, J=1.8 Hz), 8.22 (brs, 4H), 8.56 (d, 1H, J=0.9 Hz), 8.84 (s, 1H); MS (ESI) m/z=399 (MH⁺).

### Example 380

### N-[1-(3-Chloro-6-furan-3-yl-8-trifluoromethyl-imidazo[1,2-a]pyridine-2-carbonyl)-pyrrolidin-3-yl]-methanesulfonamide (Compound 480)

To a solution of (3-amino-pyrrolidin-1-yl)-(3-chloro-6-furan-3-yl-8-trifluoromethyl-imidazo[1,2-a]pyridin-2-yl)-methanone hydrochloride (compound 479, 50 mg, 0.115 mmol) in DMF (0.8 mL) was added N,N-diisopropylethylamine (80 μL, 0.459 mmol) and methanesulfonyl chloride (10.7 μL, 0.137 mmol). After 30 min, methanesulfonyl chloride (10 μL) was added. After 15 min, the mixture was diluted with EtOAc (20 mL) was washed with saturated aqueous NaHCO₃ (10 mL), then brine (10 mL). The extracts were dried (Na₂SO₄), filtered and concentrated. Column chromatography [CH₂Cl₂/MeOH (97:3 v/v)] of the crude gave N-[1-(3-chloro-6-furan-3-yl-8-trifluoromethyl-imidazo[1,2-a]pyridine-2-carbonyl)-pyrrolidin-3-yl]-methanesulfonamide (40.6 mg, 74%) as a white powder. ¹H NMR (d₆-DMSO, 300 MHz) [symbol] 1.82-2.00 (m, 1H), 2.11-2.24 (m, 1H), 2.93 (s, 1.5H), 2.99 (s, 1.5H), 3.42-4.10 (m, 5H), 7.32 (d, 1H, J=1.7 Hz), 7.45 (dd, 1H, J=4.1, 6.2 Hz), 7.84 (t, 1H, J=1.7 Hz), 8.20 (s, 1H), 8.56 (s, 1H), 8.82 (s, 1H); MS (ESI) m/z=477 (MH⁺).

### Example 381

### N-[1-(3-Chloro-6-furan-3-yl-8-trifluoromethyl-imidazo[1,2-a]pyridine-2-carbonyl)-pyrrolidin-3-yl]-acetamide (Compound 481)

To a solution of (3-amino-pyrrolidin-1-yl)-(3-chloro-6-furan-3-yl-8-trifluoromethyl-imidazo[1,2-a]pyridin-2-yl)-methanone hydrochloride (compound 479, 50 mg, 0.115 mmol) in DMF (0.8 mL) was added N,N-diisopropylethylamine (80 L, 0.459 mmol) and acetic anhydride (13 L, 0.138 mmol). After 30 min, the mixture was diluted with EtOAc (20 mL) and washed with saturated aqueous NaHCO₃ (10 mL), then brine (10 mL). The extracts were dried (Na₂SO₄), filtered and concentrated. Column chromatography [CH₂Cl₂/MeOH (95:5 v/v)] of the crude gave N-[1-(3-chloro-6-furan-3-yl-8-trifluoromethyl-imidazo[1,2-a]pyridine-2-carbonyl)-pyrrolidin-3-yl]-acetamide (39.5 mg, 78%) as a white powder. ¹H NMR (d₆-DMSO, 300 MHz) [symbol] 1.78 (s, 1.5H), 1.83 (s, 1.5H), 1.76-1.90 (m, 1H), 2.02-2.16 (m, 1H), 3.34-4.02 (m, 4H), 4.22-4.32 (m, 1H), 7.32 (m, 1H), 7.84 (t, 1H, J=1.8 Hz), 8.14 (d, 1H, J=6.7 Hz), 8.20 (s, 1H), 8.55 (d, 1H, J=0.6 Hz), 8.81 (brs, 1H); MS (ESI) m/z=441 (MH⁺).

### Example 382

### Cyclopropanecarboxylic Acid[1-(3-chloro-6-furan-3-yl-8-trifluoromethyl-imidazo[1,2-a]pyridine-2-carbonyl)-pyrrolidin-3-yl]-amide (Compound 482)

Using similar method as for the preparation of compound 481, acylation of (3-amino-pyrrolidin-1-yl)-(3-chloro-6-furan-3-yl-8-trifluoromethyl-imidazo[1,2-a]pyridin-2-yl)-methanone hydrochloride compound 479) with cyclopropanecarboxylic acid gave cyclopropanecarboxylic acid [1-(3-chloro-6-furan-3-yl-8-trifluoromethyl-imidazo[1,2-a]pyridine-2-carbonyl)-pyrrolidin-3-yl]-amide as a white powder. ¹H NMR (d₆-DMSO, 300 MHz) [symbol] 0.60-0.73 (m, 4H), 1.48-1.62 (m, 1H), 1.76-1.92 (m, 1H), 2.04-2.16 (m, 1H), 3.36-4.02 (m, 4H), 4.24-4.36 (m, 1H), 7.31 (m, 1H), 7.84 (m, 1H), 8.20 (m, 1H), 8.36 (d, 0.5H, J=6.7 Hz), 8.41 (d, 0.5H, J=6.5 Hz), 8.55 (s, 1H), 8.81 (brs, 0.51H), 8.81 (brs, 0.5H); MS (ESI) m/z=467 (MH⁺).

### Example 383

### 3-(6-Furan-3-yl-8-trifluoromethyl-imidazo[1,2-a]pyridin-2-ylmethyl)-5-phenyl-oxazolidin-2-one (Compound 483)

### Step 1: 5-Phenyl-oxazolidin-2-one

To a solution of 2-amino-1-phenylethanol (1 g, 7.29 mmol) in CH₂Cl₂ (75 mL) was added imidazole (248 mg, 3.64 mmol) followed by N,N-carbonyldiimidazole (1.241 g, 7.65 mmol). After 3 days, the mixture was washed with aqueous hydrochloride (1N, 2×50 mL). The extracts was filtered through a pad of silica gel and washed with EtOAc (200 mL). Concentration of the solvent gave 5-phenyl-oxazolidin-2-one (1.026 g, 86%) as a white solid. ¹H NMR (d₆-DMSO, 300 MHz) [symbol] 3.33 (ddd, 1H, J=0.8, 7, 8.8 Hz), 3.88 (dt, 1H, J=0.6, 8.8 Hz), 5.59 (dd, 1H, J=7.3, 8.5 Hz), 7.33-7.46 (m, 5H), 7.68 (s, 1H); MS (ESI) m/z=164.1 (MH⁺).

### Step 2: (6-Furan-3-yl-8-trifluoromethyl-imidazo[1,2-a]pyridin-2-yl)-methanol

To a solution of 6-furan-3-yl-8-trifluoromethyl-imidazo[1,2-a]pyridine-2-carboxylic acid (500 mg, 1.688 mmol) in THF (20 mL) at 0° C. was added a solution of borane tetrahydrofuran complex (1M in THF, 5.1 mL, 5.06 mmol). After 10 min, the ice-water bath was removed and the mixture was allowed to stir at room temperature for 9 hours. Water was added slowly to quench the reaction which was then diluted with EtOAc (100 mL). The organic layer was washed with saturated aqueous solution of NaHCO₃ (20 mL), then brine (20 mL). The organic layer was filtered through a pad of silica gel and the solvent was concentrated under vacuo to give (6-furan-3-yl-8-trifluoromethyl-imidazo[1,2-a]pyridin-2-yl)-methanol (272 mg, 57%) as a solid. ¹H NMR (d₆-DMSO, 300 MHz) [symbol] 4.63 (d, 2H, J=5.3 Hz), 5.33 (t, 1H, J=5.3 Hz), 7.07 (dd, 1H, J=0.8, 2 Hz), 7.82 (t, 1H, J=1.8 Hz), 7.90 (s, 1H), 7.95 (s, 1H), 8.38 (s, 1H), 9.12 (s, 1H); MS (ESI) m/z=283.1 (MH⁺).

### Step 3: Methanesulfonic acid 6-furan-3-yl-8-trifluoromethyl-imidazo[1,2-a]pyridin-2-ylmethyl Ester

To a solution of (6-furan-3-yl-8-trifluoromethyl-imidazo[1,2-a]pyridin-2-yl)-methanol (270 mg, 0.957 mmol) in DMF (5 mL) at 0° C. was added N,N-diisopropylethylamine (0.5 mL, 2.87 mmol) followed by dropwise addition of methanesulfonyl chloride (81.8 μL, 1.05 mmol). After 1 hour, the mixture was diluted with EtOAc (50 mL) and washed with saturated aqueous solution of NH₄Cl (25 mL), then brine (20 mL). The extracts were dried (Na₂SO₄), filtered and concentrated. Column chromatography [n-hex/EtOAc (5:4 v/v)] of the crude product gave methanesulfonic acid 6-furan-3-yl-8-trifluoromethyl-imidazo[1,2-a]pyridin-2-ylmethyl ester (164 mg, 48%) as a white solid. ¹H NMR (d₆-DMSO, 300 MHz) [symbol] 3.28 (s, 3H), [symbol] 5.43 (s, 2H), 7.04 (dd, 1H, J=0.9, 1.7 Hz), 7.83 (t, 1H, J=1.7 Hz), 8.07 (s, 1H), 8.20 (s, 1H), 8.42 (s, 1H), 9.17 (s, 1H); MS (ESI) m/z=361 (MH⁺).

### Step 4: 3-(6-Furan-3-yl-8-trifluoromethyl-imidazo[1,2-a]pyridin-2-ylmethyl)-5-phenyl-oxazolidin-2-one (compound 483)

To a solution of 5-phenyl-oxazolidin-2-one (34 mg, 0.208 mmol) in DMF (1.5 mL) at 0° C. was added NaH (60%, 6 mg, 0.222 mmol). After 10 min, methanesulfonic acid 6-furan-3-yl-8-trifluoromethyl-imidazo[1,2-a]pyridin-2-ylmethyl ester (50 mg, 0.139 mmol) was added in one portion. After 80 min, water (10 mL) was added and the mixture was diluted with EtOAc (20 mL). The organic phase was separated, dried (Na₂SO₄), filtered and concentrated. The crude material was purified by RP-HPLC (20-99% ACN gradient) and further purified by silica gel chromatography [EtOAc/n-hex (3:2 v/v) followed by EtOAc/n-hex (2:1 v/v)] to give 3-(6-furan-3-yl-8-trifluoromethyl-imidazo[1,2-a]pyridin-2-ylmethyl)-5-phenyl-oxazolidin-2-one (15.1 mg, 25%) as a white powder. ¹H NMR (d₆-DMSO, 300 MHz) [symbol] 3.55 (dd, 1H, J=7.3, 9.1 Hz), 4.02 (t, 1H, J=8.8 Hz), 4.57 (d, 1H, J=15.5 Hz), 4.63 (d, 1H, J=15.5 Hz), 5.59 (dd, 1H, J=7.3, 8.8 Hz), 7.03 (dd, 1H, J=0.9, 1.8 Hz), 7.34-7.46 (m, 5H), 7.82 (t, 1H, J=1.8 Hz), 8.00 (s, 1H), 8.02 (s, 1H), 8.39 (s, 1H), 9.11 (s, 1H); MS (ESI) m/z=428.2 (MH⁺).

### Example 384

### 3-Iodo-6-(1H-pyrazol-4-yl)-8-trifluoromethyl-imidazo[1,2-a]pyridine-2-carboxylic Acid (Compound 484)

### Step 1: 6-(1-tert-Butoxycarbonyl-1H-pyrazol-4-yl)-8-trifluoromethyl-imidazo[1,2-a]pyridine-2-carboxylic Acid Methyl Ester

DMF (155 mL) was added under argon to a mixture of 6-bromo-8-trifluoromethyl-imidazo[1,2-a]pyridine-2-carboxylic acid methyl ester (5 g, 15.47 mmol), 4-(4,4,5,5-tetramethyl-[1,3,2]dioxaborolan-2-yl)-pyrazole-1-carboxylic acid tert-butyl ester (22.75 g, 77.40 mmol), tetrakis(triphenylphosphine)palladium(0) (1.79 g, 1.55 mmol), and cesium carbonate (50.4 g, 155 mmol) and reaction was heated to 80° C. for 20 min. After cooling in a water bath, the solvent was removed in-vacuo. To the resulting residue was added H₂O and diethyl ether and sample was sonicated for 30 min. The precipitate was filtered and washed successively with H₂O and diethyl ether, and then air dried to obtain 6-(1-tert-butoxycarbonyl-1H-pyrazol-4-yl)-8-trifluoromethyl-imidazo[1,2-a]pyridine-2-carboxylic acid methyl ester (5.61 g, 90%) as a beige solid. MS (ESI) m/z=410.9 (MH⁺).

### Step 2: 6-(1H-Pyrazol-4-yl)-8-trifluoromethyl-imidazo[1,2-a]pyridine-2-carboxylic Acid

To a solution of 6-(1-tert-butoxycarbonyl-1H-pyrazol-4-yl)-8-trifluoromethyl-imidazo[1,2-a]pyridine-2-carboxylic acid methyl ester (3.34 g, 8.14 mmol) in THF and DMF (5:1 v/v, 97 mL) at room temperature was added aqueous NaOH solution (1 M, 32 mL). After 4 hours, the pH was adjusted to 4 with aqueous citric acid (1 M). The residual THF was removed and the resulting precipitate was filtered and washed successively with H₂O and diethyl ether, and then air dried to obtain 6-(1H-pyrazol-4-yl)-8-trifluoromethyl-imidazo[1,2-a]pyridine-2-carboxylic acid (2.24 g, 93%) as a beige solid. MS (ESI) m/z=297.0 (MH⁺).

### Step 3: 3-Iodo-6-(1H-pyrazol-4-yl)-8-trifluoromethyl-imidazo[1,2-a]pyridine-2-carboxylic Acid (Compound 484)

N-iodosuccinimide (5.11 g, 22.7 mmol) was added in 9 portions to a solution of 6-(1H-pyrazol-4-yl)-8-trifluoromethyl-imidazo[1,2-a]pyridine-2-carboxylic acid (2.242 g, 7.57 mmol) in DMF (76 mL) at room temperature. After 24 hours, the reaction was quenched with 5% aqueous NaHSO₃. The precipitate was filtered and washed successively with H₂O and diethyl ether, and then air dried to obtain 3-iodo-6-(1H-pyrazol-4-yl)-8-trifluoromethyl-imidazo[1,2-a]pyridine-2-carboxylic acid (2.312 g, 72%) as a beige solid. MS (ESI) m/z=423.1 (MH⁺).

### Example 385

### 3,6-Bis-(1H-pyrazol-4-yl)-8-trifluoromethyl-imidazo[1,2-a]pyridine-2-carboxylic acid (thiophen-2-ylmethyl)-amide (Compound 485)

### Step 1: 3,6-Bis-(1H-pyrazol-4-yl)-8-trifluoromethyl-imidazo[1,2-a]pyridine-2-carboxylic Acid Methyl Ester

DMF (14 mL) was added to a mixture of 6-bromo-3-chloro-8-trifluoromethyl-imidazo[1,2-a]pyridine-2-carboxylic acid methyl ester (0.50 g, 1.40 mmol), 4-(4,4,5,5-tetramethyl-[1,3,2]dioxaborolan-2-yl)-pyrazole-1-carboxylic acid tert-butyl ester (2.06 g, 7.0 mmol), tetrakis(triphenylphosphine)palladium(0) (0.162 g, 0.14 mmol), and saturated aqueous NaHCO₃ (1.9 mL) and the reaction was heated at 120° C. for 20 min under microwave conditions. The solvent was removed in-vacuo, and to the resulting residue was added H₂O and diethyl ether and sample was sonicated for 30 min. The precipitate was filtered and washed successively with H₂O and diethyl ether, and then air dried to obtain 3,6-bis-(1H-pyrazol-4-yl)-8-trifluoromethyl-imidazo[1,2-a]pyridine-2-carboxylic acid methyl ester (819 mgs, 85%) as a brown solid. MS (ESI) m/z=377.0 (MH⁺).

### Step 2: 3,6-Bis-(1H-pyrazol-4-yl)-8-trifluoromethyl-imidazo[1,2-a]pyridine-2-carboxylic Acid

An aqueous solution of NaOH (1M, 4.4 mL) was added slowly to a suspension of 3,6-bis-(1H-pyrazol-4-yl)-8-trifluoromethyl-imidazo[1,2-a]pyridine-2-carboxylic acid methyl ester (819 mg, 2.3 mmol) in THF (24 mL) at room temperature. After stirring over night, the pH was adjusted to 4 with aqueous citric acid (1M). The resulting precipitate was filtered and washed successively with H₂O and diethyl ether, and then air dried to obtain 3,6-bis-(1H-pyrazol-4-yl)-8-trifluoromethyl-imidazo[1,2-a]pyridine-2-carboxylic acid (622 mg, 72%) as a beige solid. MS (ESI) m/z=363.0 (MH⁺).

### Step 3: 3,6-Bis-(1H-pyrazol-4-yl)-8-trifluoromethyl-imidazo[1,2-a]pyridine-2-carboxylic Acid (thiophen-2-ylmethyl)-amide (compound 485)

Prepared using standard HATU coupling conditions. ¹H NMR (d₆-DMSO, 300 MHz) [symbol] 4.63 (d, 2H, J=6.2, Hz), 6.94-6.96 (m, 1H), 7.02-7.03 (m, 1H), 8.11 (s, 1H), 8.17 (broad s, 4H), 8.59 (s, 2H), 8.69 (t, 1H, J=6.7 Hz); MS (ESI) m/z=458.1 (MH⁺).

### Example 386

### [3,6-Bis-(1H-pyrazol-4-yl)-8-trifluoromethyl-imidazo[1,2-a]pyridin-2-yl]-[3-(3-fluoro-phenyl)-pyrrolidin-1-yl]-methanone (Compound 486)

Prepared using standard HATU coupling conditions. ¹H NMR (d₆-DMSO, 300 MHz) [symbol] 1.97-2.07 (m, 1H), 2.20-2.33 (m, 1H), 3.37-3.79 (m, 4H), 3.93-4.05 (m, 1H), 7.01-7.22 (m, 3H), 7.28-7.41 (m, 1H), 8.07 (d, 1H, J=7.0 Hz), 8.18 (s, 2H), 8.28 (s, 2H), 8.63 (d, 1H, J=4.4 Hz); MS (ESI) m/z=510.0 (MH⁺).

### Example 387

### (3-Chloro-6-furan-3-yl-8-trifluoromethyl-imidazo[1,2-a]pyridin-2-yl)-(2-phenyl-azetidin-1-yl)-methanone (Compound 487)

Prepared using standard HATU coupling conditions as a 2:1 mixture of rotomers. NMR (d₆-DMSO, 300 MHz) [symbol] 1.99-2.08 (m, 0.5H), 2.11-2.22 (m, 1H), 2.73-2.85 (m, 1H), 2.90-2.96 (m, 0.5H), 4.11-4.20 (m, 0.5H), 4.30-4.39 (m, 0.5H), 4.55-4.63 (m, 1H), 4.70-4.79 (m, 1H), 5.49 (dd, 1H, J=6.2, 8.8 Hz), 5.97 (dd, 0.5H, J=4.7, 8.8 Hz), 7.07-7.45 (m, 6H), 7.80 (t, 0.5H, J=1.8 Hz), 7.84 (t, 1H, J=1.5 Hz), 8.10 (s, 0.5H), 8.22 (s, 1H), 8.49 (s, 0.5H), 8.57 (s, 1H), 8.65 (s, 0.5H), 8.82 (s, 1H); MS (ESI) m/z=446.0 (MH⁺).

### Example 388

### (3-Chloro-6-furan-3-yl-8-trifluoromethyl-imidazo[1,2-a]pyridin-2-yl)-(2-phenyl-azetidin-1-yl)-methanone (Compound 488)

Prepared using standard HATU coupling conditions. ¹H NMR (d₆-DMSO, 300 MHz) [symbol] 4.07-4.17 (m, 2H), 4.49-4.58 (m, 2H), 5.07 (dd, 1H, J=7.6, 10.3 Hz), 7.33 (dd, 1H, J=0.9, 2.1 Hz), 7.65-7.75 (m, 4H), 7.84 (t, 1H, J=1.5 Hz), 7.95 (s, 1H), 8.21 (s, 1H), 8.56 (s, 1H), 8.83 (s, 1H); MS (ESI) m/z=514.0 (MH⁺).

### Example 389

### [1-(3-Chloro-6-furan-3-yl-8-trifluoromethyl-imidazo[1,2-a]pyridine-2-carbonyl)-azetidin-3-yl]-carbamic Acid Tert-butyl Ester (Compound 489)

Prepared using standard HATU coupling conditions. ¹H NMR (d₆-DMSO, 300 MHz) [symbol] 1.39 (s, 9H), 3.89-3.92 (m, 1H), 4.25-4.38 (m, 3H), 4.77 (t, 1H, J=8.2 Hz), 7.31-7.32 (m, 1H), 7.61-7.63 (m, 1H), 7.84 (t, 1H, J=1.8 Hz), 8.21 (s, 1H), 8.56 (s, 1H), 8.81 (s, 1H); MS (ESI) m/z=485.1 (MH⁺).

### Example 390

### (3-Amino-azetidin-1-yl)-(3-chloro-6-furan-3-yl-8-trifluoromethyl-imidazo[1,2-a]pyridin-2-yl)-methanone Hydrochloride (Compound 490)

A solution of hydrogen chloride in dioxane (4M, 2 mL) was added to [1-(3-chloro-6-furan-3-yl-8-trifluoromethyl-imidazo[1,2-a]pyridine-2-carbonyl)-azetidin-3-yl]-carbamic acid tert-butyl ester (compound 489 in Example 389, 111 mg, 0.23 mmol) and reaction was sonicated. After 2 hours, the precipitate was filtered to give (3-amino-azetidin-1-yl)-(3-chloro-6-furan-3-yl-8-trifluoromethyl-imidazo[1,2-a]pyridin-2-yl)-methanone hydrochloride (60 mg, 68%) as a white solid. ¹H NMR (d₆-DMSO, 300 MHz) [symbol] 4.04-4.09 (m, 2H), 4.29-4.36 (m, 1H), 4.59 (dd, 1H, J=6.7, 11.7 Hz), 4.83 (dd, 1H, J=4.4, 11.7 Hz), 7.33 (d, 1H, J=1.8 Hz), 7.85 (t, 1H, J=1.8 Hz), 8.24 (s, 1H), 8.41 (s, 3H), 8.57 (s, 1H), 8.84 (s, 1H); MS (ESI) m/z=384.9 (MH⁺).

### Example 391

### N-[1-(3-Chloro-6-furan-3-yl-8-trifluoromethyl-imidazo[1,2-a]pyridine-2-carbonyl)-azetidin-3-yl]-methanesulfonamide (Compound 491)

Methanesulfonyl chloride (10 μL, 0.13 mmol) was added to a solution of N,N-diisopropylethylamine (184 μL) and (3-amino-azetidin-1-yl)-(3-chloro-6-furan-3-yl-8-trifluoromethyl-imidazo[1,2-a]pyridin-2-yl)-methanone (60 mg, 0.143 mmol) in DMF (715 μL). After 2 hours, water was added and the precipitate was filtered and subjected to silica chromatography to give N-[1-(3-chloro-6-furan-3-yl-8-trifluoromethyl-imidazo[1,2-a]pyridine-2-carbonyl)-azetidin-3-yl]-methanesulfonamide (30 mg, 45%) as a white solid. ¹H NMR (d₆-DMSO, 300 MHz) [symbol] 2.94 (s, 3H), 3.95 (dd, 1H, J=4.7, 9.7 Hz), 4.28-4.46 (m, 3H), 4.86 (dd, 1H, J=6.7, 10.5 Hz), 7.32 (d, 1H, J=1.2 Hz), 7.84 (t, 1H, J=1.2 Hz), 7.91 (d, 1H, J=8.2 Hz), 8.22 (s, 1H), 8.56 (s, 1H) 8.82 (s, 1H); MS (ESI) m/z=463.0 (MH⁺).

The compounds in Examples 392-403 were made by the same method as that used in Example 391 using the appropriate sulfonyl chloride or acid chloride.

### Example 392

### N-[1-(3-Chloro-6-furan-3-yl-8-trifluoromethyl-imidazo[1,2-a]pyridine-2-carbonyl)-azetidin-3-yl]-benzenesulfonamide (Compound 492)

White solid (14 mg, 19%). ¹H NMR (d₆-DMSO, 300 MHz) [symbol] 3.64-3.70 (m, 1H), 4.05-4.21 (m, 3H), 4.52-4.57 (m, 1H), 7.31 (d, 1H, J=1.6 Hz), 7.59-7.72 (m, 3H), 7.81-7.84 (m, 3H), 8.19 (s, 1H), 8.43 (d, 1H, J=8.2 Hz), 8.55 (s, 1H), 8.78 (s, 1H); MS (ESI) m/z=525.0 (MH⁺).

### Example 393

### N-[1-(3-Chloro-6-furan-3-yl-8-trifluoromethyl-imidazo[1,2-a]pyridine-2-carbonyl)-azetidin-3-yl]-C-phenyl-methanesulfonamide (Compound 493)

White solid (27 mg, 35%). ¹H NMR (d₆-DMSO, 300 MHz) [symbol] 3.85 (dd, 1H, J=5.0, 9.4 Hz), 4.16-4.39 (m, 5H), 4.72 (dd, 1H, J=7.6, 10.5 Hz), 7.32 (dd, 1H, J=0.9, 1.7 Hz), 7.38 (s, 5H), 7.84 (t, 1H, J=1.5 Hz), 8.01 (d, 1H, J=8.2 Hz), 8.22 (s, 1H), 8.56 (s, 1H), 8.81 (s, 1H); MS (ESI) m/z=539.0 (MH⁺).

### Example 394

### N-[1-(3-Chloro-6-furan-3-yl-8-trifluoromethyl-imidazo[1,2-a]pyridine-2-carbonyl)-azetidin-3-yl]-2-fluoro-benzenesulfonamide (Compound 494)

White solid (35 mg, 68%). ¹H NMR (d₆-DMSO, 300 MHz) [symbol] 3.71-3.75 (m, 1H), 3.99 (dd, 1H, J=5.0, 11.4 Hz), 4.21-4.26 (m, 2H), 4.49 (dd, 1H, J=7.3, 11.7 Hz), 7.30 (dd, 1H, J=0.9, 1.8 Hz), 7.43-7.49 (m, 2H), 7.83 (t, 1H, J=1.8 Hz), 7.87-7.91 (m, 2H), 8.19 (s, 1H), 8.46 (d, 1H, J=8.2 Hz), 8.55 (s, 1H), 8.78 (s, 1H); MS (ESI) m/z=543.0 (MH⁺).

### Example 395

### N-[1-(3-Chloro-6-furan-3-yl-8-trifluoromethyl-imidazo[1,2-a]pyridine-2-carbonyl)-azetidin-3-yl]-3-fluoro-benzenesulfonamide (compound 495)

White solid (35 mg, 68%). ¹H NMR (d₆-DMSO, 300 MHz) [symbol] 3.71-3.76 (m, 1H), 4.07 (dd, 1H, J=4.1, 10.5 Hz), 4.19-4.24 (m, 2H), 4.55 (dd, 1H, J=6.1, 10.5 Hz), 7.30 (dd, 1H, J=0.9, 2.1 Hz), 7.54-7.74 (m, 4H), 7.83 (t, 1H, J=1.8 Hz), 8.19 (s, 1H), 8.55 (s, 1H), 8.58 (d, 1H, J=7.5 Hz), 8.78 (s, 1H); MS (ESI) m/z=543.0 (MH⁺).

### Example 396

### N-[1-(3-Chloro-6-furan-3-yl-8-trifluoromethyl-imidazo[1,2-a]pyridine-2-carbonyl)-azetidin-3-yl]-4-fluoro-benzenesulfonamide (Compound 496)

White solid (33 mg, 64%). ¹H NMR (d₆-DMSO, 300 MHz) [symbol] 3.82-3.85 (m, 1H), 4.18-4.26 (m, 3H), 4.58-4.64 (m, 1H), 7.31 (dd, 1H, J=0.9, 2.1 Hz), 7.40-7.51 (m, 2H), 7.72-7.86 (m, 3H), 8.19 (s, 1H), 8.55 (s, 1H), 8.77-8.79 (m, 2H); MS (ESI) m/z=543.0 (MH⁺).

### Example 397

### Propane-2-sulfonic acid [1-(3-chloro-6-furan-3-yl-8-trifluoromethyl-imidazo[1,2-a]pyridine-2-carbonyl)-azetidin-3-yl]-amide (Compound 497)

White solid (15 mg, 13%). ¹H NMR (d₆-DMSO, 300 MHz) [symbol] 1.23 (dd, 6H, J=1.8, 6.7 Hz), 3.15 (m, 1H), 3.94 (dd, 1H, J=4.7, 9.4 Hz), 4.26-4.47 (m, 3H), 4.83 (dd, 1H, J=7.6, 10.0 Hz), 7.31 (dd, 1H, J=0.9, 1.8 Hz), 7.83 (t, 1H, J=1.8 Hz), 7.92 (d, 1H, J=8.5 Hz), 8.21 (s, 1H), 8.55 (s, 1H), 8.81 (s, 1H); MS (ESI) m/z=491.0 (MH⁺).

### Example 398

### Cyclopropanesulfonic Acid[1-(3-chloro-6-furan-3-yl-8-trifluoromethyl-imidazo[1,2-a]pyridine-2-carbonyl)-azetidin-3-yl]-amide (Compound 498)

White solid (35 mg, 75%). ¹H NMR (d₆-DMSO, 300 MHz) [symbol] 0.92-0.98 (m, 4H), 3.98 (dd, 1H, J=5.0, 10.3 Hz), 4.24-4.50 (m, 3H), 4.86 (dd, 1H, J=7.9, 10.0 Hz), 7.32 (dd, 1H, J=0.9, 2.1 Hz), 7.84 (t, 1H, J=1.8 Hz), 7.97 (d, 1H, J=9.1 Hz), 8.22 (s, 1H), 8.56 (s, 1H), 8.82 (s, 1H); MS (ESI) m/z=489.0 (MH⁺).

### Example 399

### Thiophene-2-sulfonic acid [1-(3-chloro-6-furan-3-yl-8-trifluoromethyl-imidazo[1,2-a]pyridine-2-carbonyl)-azetidin-3-yl]-amide (Compound 499)

White solid (37 mg, 73%). ¹H NMR (d₆-DMSO, 300 MHz) [symbol] 2.53-2.58 (m, 1H), 3.69-3.77 (m, 1H), 4.15-4.23 (m, 3H), 4.63-4.68 (m, 1H), 7.22 (dd, 1H, J=3.8, 5.0 Hz), 7.31 (dd, 1H, J=0.9, 2.1 Hz), 7.65 (dd, 1H, J=1.5, 3.8 Hz), 7.83 (t, 1H, J=1.5 Hz), 8.00 (dd, 1H, J=1.5, 5.0 Hz), 8.20 (s, 1H), 8.55 (s, 1H), 8.64 (d, 1H, J=7.6 Hz), 8.79 (s, 1H); MS (ESI) m/z=531.0 (MH⁺).

### Example 400

### Ethanesulfonic Acid[1-(3-chloro-6-furan-3-yl-8-trifluoromethyl-imidazo[1,2-a]pyridine-2-carbonyl)-azetidin-3-yl]-amide (Compound 500)

White solid (24 mg, 53%). ¹H NMR (d₆-DMSO, 300 MHz) [symbol] 1.20 (t, 3H, J=7.3 Hz), 4.57 (q, 2H, J=7.3 Hz), 3.94 (dd, 1H, J=5.3, 10.0 Hz), 4.23-4.46 (m, 3H), 4.85 (dd, 1H, J=8.2, 10.0 Hz), 7.32 (dd, 1H, J=0.6, 1.8 Hz), 7.84 (t, 1H, J=1.8 Hz), 7.94 (d, 1H, J=8.2 Hz), 8.21 (s, 1H), 8.56 (s, 1H), 8.82 (s, 1H); MS (ESI) m/z=477.0 (MH⁺).

### Example 401

### N-[1-(3-Chloro-6-furan-3-yl-8-trifluoromethyl-imidazo[1,2-a]pyridine-2-carbonyl)-azetidin-3-yl]-acetamide (Compound 501)

White solid (36 mg, 60%). ¹H NMR (d₆-DMSO, 300 MHz) [symbol] 1.84 (s, 3H), 3.87 (dd, 1H, J=5.0, 10.5 Hz), 4.29-4.41 (m, 2H), 4.49-4.55 (m, 1H), 4.79 (dd, 1H, J=8.2, 9.7 Hz), 7.32 (dd, 1H, J=0.9, 1.8 Hz), 7.84 (t, 1H, J=1.8 Hz), 8.21 (s, 1H), 8.56 (s, 1H), 8.58 (d, 1H, J=7.0 Hz), 8.81 (s, 1H); MS (ESI) m/z=427.0 (MH⁺).

### Example 402

### N-[1-(3-Chloro-6-furan-3-yl-8-trifluoromethyl-imidazo[1,2-a]pyridine-2-carbonyl)-azetidin-3-yl]-2-phenyl-acetamide (Compound 502)

White solid (44 mg, 63%). ¹H NMR (d₆-DMSO, 300 MHz), [symbol] 3.44 (s, 3H), 3.88 (dd, 1H, J=5.6, 10.5 Hz), 4.30-4.43 (m, 2H), 4.50-4.56 (m, 1H), 4.80 (dd, 1H, J=10.3, 18.5 Hz), 7.20-7.33 (m, 5H), 7.84 (t, 1H, J=1.8 Hz), 8.21 (s, 1H), 8.56 (s, 1H), 8.82-8.83 (m, 2H); MS (ESI) m/z=503.1 (MH⁺).

### Example 403

### N-[1-(3-Chloro-6-furan-3-yl-8-trifluoromethyl-imidazo[1,2-a]pyridine-2-carbonyl)-azetidin-3-yl]-benzamide (Compound 503)

White solid (32 mg, 47%). MS (ESI) m/z=489.0 (MH⁺).

The compounds in Examples 404-407 were made by the same method as that used in Example 391 using the appropriate carbamoyl chloride or isocyanate.

### Example 404

### 3-[1-(3-Chloro-6-furan-3-yl-8-trifluoromethyl-imidaz [1,2-a]pyridine-2-carbonyl)-azetidin-3-yl]-1,1-dimethyl-urea (Compound 504)

White solid (34 mg, 73%). ¹H NMR (d₆-DMSO, 300 MHz) [symbol] 2.67 (s, 6H), 3.93 (dd, 1H, J=5.3, 10.0 Hz), 4.18-4.36 (m, 2H), 4.43 (dd, 1H, J=5.3, 10.8 Hz), 4.81 (dd, 1H, J=8.5, 10.3 Hz), 7.32 (dd, 1H, J=0.6, 1.8 Hz), 7.84 (t, 1H, J=1.5 Hz), 7.97 (d, 1H, J=8.5 Hz), 8.22 (s, 1H), 8.56 (s, 1H), 8.82 (s, 1H); MS (ESI) m/z=492.0 (MH⁺).

### Example 405

### Morpholine-4-carboxylic acid [1-(3-chloro-6-furan-3-yl-8-trifluoromethyl-imidazo[1,2-a]pyridine-2-carbonyl)-azetidin-3-yl]-amide (Compound 505)

White solid (33 mg, 47%). MS (ESI) m/z=498.3 (MH⁺).

### Example 406

1-[1-(3-Chloro-6-furan-3-yl-8-trifluoromethyl-imidazo[1,2-a]pyridine-2-carbonyl)-azetidin-3-yl]-3-phenyl-urea (Compound 506)

White solid (33 mg, 47%). MS (ESI) m/z=504.3 (MH⁺).

### Example 407

### 1-Benzyl-3-[1-(3-chloro-6-furan-3-yl-8-trifluoromethyl-imidazo[1,2-a]pyridine-2-carbonyl)-azetidin-3-yl]-urea (Compound 507)

White solid (23 mg, 32%). MS (ESI) m/z=518.3 (MH⁺).

### Example 408

### 3-[(3-Chloro-6-furan-3-yl-8-trifluoromethyl-imidazo[1,2-a]pyridine-2-carbonyl)-amino]-azetidine-1-carboxylic acid tert-butyl Ester (Compound 508)

Prepared using standard HATU coupling conditions. ¹H NMR (d₆-DMSO, 300 MHz) [symbol] 1.40 (s, 9H), 3.99-4.10 (m, 4H), 4.70-4.77 (m, 1H), 7.32-7.33 (m, 1H), 7.84 (t, 1H, J=1.8 Hz), 8.23 (s, 1H), 8.57 (s, 1H), 8.82 (s, 1H), 8.93 (d, 1H, J=7.9 Hz); MS (ESI) m/z=485.2 (MH⁺).

### Example 409

### 3-Chloro-6-furan-3-yl-8-trifluoromethyl-imidazo[1,2-a]pyridine-2-carboxylic Acid Azetidin-3-ylamide Hydrochloride (Compound 509)

A solution of hydrogen chloride in dioxane (4M, 18 mL) was added to 3-[(3-chloro-6-furan-3-yl-8-trifluoromethyl-imidazo[1,2-a]pyridine-2-carbonyl)-amino]-azetidine-1-carboxylic acid tert-butyl ester (compound 508 in Example 408, 950 mg, 1.96 mmol) and reaction was sonicated. After 2 hours the precipitate was filtered to give 3-chloro-6-furan-3-yl-8-trifluoromethyl-imidazo[1,2-a]pyridine-2-carboxylic acid azetidin-3-ylamide hydrochloride (878 mg, 100%) as a white solid. ¹H NMR (d₆-DMSO, 300 MHz) [symbol] 4.10-4.27 (m, 4H), 4.81-4.88 (m, 1H), 7.33 (dd, 1H, J=0.6, 1.8 Hz), 7.85 (t, 1H, J=1.8 Hz), 8.25 (s, 1H), 8.57 (t, 1H, J=1.2 Hz), 8.65 (s, 2H), 8.83 (s, 1H), 9.00 (d, 1H, J=7.3 Hz); MS (ESI) m/z=385.0 (MH⁺).

### Example 410

### 3-Chloro-6-furan-3-yl-8-trifluoromethyl-imidazo[1,2-a]pyridine-2-carboxylic Acid (1-methanesulfonyl-azetidin-3-yl)-amide (Compound 510)

Methanesulfonyl chloride (10 μL, 0.13 mmol) was added to a solution of N,N-diisopropylethylamine (184 μL) and 3-chloro-6-furan-3-yl-8-trifluoromethyl-imidazo[1,2-a]pyridine-2-carboxylic acid azetidin-3-ylamide hydrochloride (compound 509 in Example 409, 60 mg, 0.14 mmol) in DMF (0.72 mL). After 2 hours, water was added and the precipitate was filtered and subjected to silica chromatography to give 3-chloro-6-furan-3-yl-8-trifluoromethyl-imidazo[1,2-a]pyridine-2-carboxylic acid (1-methanesulfonyl-azetidin-3-yl)-amide (17 mg, 26%) as a white solid. ¹H NMR (d₆-DMSO, 300 MHz) [symbol] 2.94 (s, 3H), 3.95 (dd, 1H, J=4.7, 9.7 Hz), 4.28-4.46 (m, 3H), 4.86 (dd, 1H, J=6.7, 10.5 Hz), 7.32 (d, 1H, J=1.2 Hz), 7.84 (t, 1H, J=1.2 Hz), 7.91 (d, 1H, J=8.2 Hz), 8.22 (s, 1H), 8.56 (s, 1H), 8.82 (s, 1H); MS (ESI) m/z=463.0 (MH⁺).

The compounds in Examples 411-415 were made by the same method as that used in Example 410 using the appropriate sulfonyl chloride or acid chloride.

### Example 411

### 3-Chloro-6-furan-3-yl-8-trifluoromethyl-imidazo[1,2-a]pyridine-2-carboxylic Acid (1-benzenesulfonyl-azetidin-3-yl)-amide (Compound 511)

White solid (42 mg, 56%). ¹H NMR (d₆-DMSO, 300 MHz) [symbol] 3.87-4.04 (m, 4H), 4.39-4.51 (m, 1H), 7.31 (dd, 1H, J=0.9, 1.9 Hz), 7.67-7.88 (m, 6H), 8.21 (s, 1H), 8.55 (s, 1H), 8.77-8.79 (m, 2H); MS (ESI) m/z=525.0 (MH⁺).

### Example 412

### 3-Chloro-6-furan-3-yl-8-trifluoromethyl-imidazo[1,2-a]pyridine-2-carboxylic Acid (1-phenylmethanesulfonyl-azetidin-3-yl)-amide (Compound 512)

White solid (19 mg, 25%). ¹H NMR (d₆-DMSO, 300 MHz) δ 3.84 (dd, 1H, J=2.3, 4.7 Hz), 4.01 (t, 1H, J=8.2 Hz), 4.14 (t, 1H, J=7.6 Hz), 4.30-4.37 (m, 2H), 4.57 (s, 1H), 4.70-4.77 (m, 1H), 7.32-7.49 (m, 6H), 7.84 (t, 1H, J=1.8 Hz), 8.24 (s, 1H), 8.57 (s, 1H), 8.83 (s, 1H), 8.92 (d, 1H, J=6.7 Hz); MS (ESI) m/z=539.0 (MH⁺).

### Example 413

### 3-Chloro-6-furan-3-yl-8-trifluoromethyl-imidazo[1,2-a]pyridine-2-carboxylic Acid (1-acetyl-azetidin-3-yl)-amide (Compound 513)

White solid (24 mg, 40%). ¹H NMR (d₆-DMSO, 300 MHz) δ 1.77 (s, 3H), 3.96 (dd, 1H, J=5.9, 9.7 Hz), 4.09 (t, 1H, J=9.1 Hz), 4.22 (dd, 1H, J=5.9, 8.5 Hz), 4.37 (t, 1H, J=8.2 Hz), 4.70-4.80 (m, 1H), 7.32 (dd, 1H, J=0.9, 1.8 Hz), 7.84 (t, 1H, J=1.8 Hz), 8.23 (s, 1H), 8.56 (s, 1H), 8.82 (s, 1H), 8.94 (d, 1H, J=7.6 Hz); MS (ESI) m/z=427.0 (MH⁺).

### Example 414

### 3-Chloro-6-furan-3-yl-8-trifluoromethyl-imidazo[1,2-a]pyridine-2-carboxylic Acid (1-phenylacetyl-azetidin-3-yl)-amide (Compound 514)

White solid (34 mg, 48%). ¹H NMR (d₆-DMSO, 300 MHz) δ 3.45 (s, 3H), 3.99-4.15 (m, 2H), 4.29 (dd, 1H, J=5.9, 8.5 Hz), 4.47 (t, 1H, J=7.9 Hz), 4.76-4.82 (m, 1H), 7.21-7.34 (m, 5H), 7.84 (t, 1H, J=1.8 Hz), 8.23 (s, 1H), 8.57 (s, 1H), 8.82 (s, 1H), 8.98 (d, 1H, J=7.6 Hz); MS (ESI) m/z=503.1 (MH⁺).

### Example 415

### 3-Chloro-6-furan-3-yl-8-trifluoromethyl-imidazo[1,2-a]pyridine-2-carboxylic Acid (1-benzoyl-azetidin-3-yl)-amide (Compound 515)

White solid (35 mg, 51%). ¹H NMR (d₆-DMSO, 300 MHz) [symbol] 4.15-4.21 (m, 1H), 4.33 (t, 1H, J=10.0 Hz), 4.43 (dd, 1H, J=5.2, 8.8 Hz), 4.59 (t, 1H, J=8.2 Hz), 4.83-4.89 (m, 1H), 7.32 (dd, 1H, J=0.9, 2.1 Hz), 7.44-7.55 (m, 3H), 7.64-7.68 (m, 2H), 7.84 (t, 1H, J=1.8 Hz), 8.23 (s, 1H), 8.56 (s, 1H), 8.82 (s, 1H), 9.00 (d, 1H, J=7.6 Hz); MS (ESI) m/z=489.1 (MH⁺).

The compounds in Examples 416-419 were made by the same method as that used in Example 410 using the appropriate carbamoyl chloride or isocyanate.

### Example 416

### 3-Chloro-6-furan-3-yl-8-trifluoromethyl-imidazo[1,2-a]pyridine-2-carboxylic Acid[1-(morpholine-4-carbonyl)-azetidin-3-yl]-amide (Compound 516)

White solid (41 mg, 70%). MS (ESI) m/z=498.2 (MH⁺).

### Example 417

### 3-Chloro-6-furan-3-yl-8-trifluoromethyl-imidazo[1,2-a]pyridine-2-carboxylic Acid (1-phenylcarbamoyl-azetidin-3-yl)-amide (Compound 517)

White solid (42 mg, 60%). MS (ESI) m/z=504.3 (MH⁺).

### Example 418

### 1-Benzyl-3-[1-(3-chloro-6-furan-3-yl-8-trifluoromethyl-imidazo[1,2-a]pyridine-2-carbonyl)-azetidin-3-yl]-urea (Compound 518)

White solid (31 mg, 43%). MS (ESI) m/z=518.2 (MH⁺).

### Example 419

### N-[1-(6-Furan-3-yl-8-trifluoromethyl-imidazazo[1,2-a]pyridine-2-carbonyl)-azetidin-3-yl]-methanesulfonamide (Compound 519)

### Step 1: [1-(6-Furan-3-yl-8-trifluoromethyl-imidazo[1,2-a]pyridine-2-carbonyl)-azetidin-3-yl]-carbamic Acid Tert-butyl Ester

Prepared using standard HATU coupling, (396 mg, 88%) as a beige solid. MS (ESI) m/z=376.1 (MH⁺).

### Step 2: (3-Amino-azetidin-1-yl)-(6-furan-3-yl-8-trifluoromethyl-imidazo[1,2-a]pyridin-2-yl)-methanone Hydrochloride

A solution of hydrogen chloride in dioxane (4M, 4 mL) was added to [1-(6-furan-3-yl-8-trifluoromethyl-imidazo[1,2-a]pyridine-2-carbonyl)-azetidin-3-yl]-carbamic acid tert-butyl ester (396 mg, 0.88 mmol) and reaction was sonicated. After 2 hours the precipitate was filtered to give (3-amino-azetidin-1-yl)-(6-furan-3-yl-8-trifluoromethyl-imidazo[1,2-a]pyridin-2-yl)-methanone hydrochloride, 379 mg, 100%) as a white solid. MS (ESI) m/z=351.0 (MH⁺).

### Step 3: N-[1-(6-Furan-3-yl-8-trifluoromethyl-imidazo[1,2-a]pyridine-2-carbonyl)-azetidin-3-yl]-methanesulfonamide (compound 519)

Methanesulfonyl chloride (22 μL) was added to a solution of N,N-diisopropylethylamine (335 μL) and (3-amino-azetidin-1-yl)-(6-furan-3-yl-8-trifluoromethyl-imidazo[1,2-a]pyridin-2-yl)-methanone (100 mg, 0.26 mmol) in DMF (1.3 mL). After 2 hours, water was added and the precipitate was filtered and subjected to silica chromatography to give N-[1-(6-furan-3-yl-8-trifluoromethyl-imidazo[1,2-a]pyridine-2-carbonyl)-azetidin-3-yl]-methanesulfonamide, (compound 519, 35 mg, 31%) as a white solid. ¹H NMR (d₆-DMSO, 300 MHz) [symbol] 2.95 (s, 3H), 3.94 (dd, 1H, J=4.7, 10.0 Hz), 4.27-4.50 (m, 3H), 4.93 (dd, 1H, J=7.9, 10.5 Hz), 7.02 (dd, 1H, J=0.9, 2.1 Hz), 7.84 (t, 1H, J=1.8 Hz), 7.91 (d, 1H, J=7.9 Hz), 8.11 (s, 1H), 8.42 (s, 1H), 8.44 (s, 1H), 9.13 (s, 1H); MS (ESI) m/z=429.0 (MH⁺).

### Example 420

### N-{1-[6-(1H-pyrazol-4-yl)-8-trifluoromethyl-imidazo[1,2-a]pyridine-2-carbonyl]-azetidin-3-yl}-methanesulfonamide (Compound 520)

### Step 1: {1-[6-(1H-pyrazol-4-yl)-8-trifluoromethyl-imidazo[1,2-a]pyridine-2-carbonyl]-azetidin-3-yl}-carbamic Acid Tert-butyl Ester

Prepared using standard HATU coupling conditions. MS (ESI) m/z=451.0 (MH⁺).

### Step 2: (3-Amino-azetidin-1-yl)-(6-(1H-pyrazol-4-yl)-8-trifluoromethyl-imidazo[1,2-a]pyridin-2-yl)-methanone Hydrochloride

A solution of hydrogen chloride in dioxane (4M, 3 mL) was added to [1-(6-(1H-pyrazol-4-yl)-8-trifluoromethyl-imidazo[1,2-a]pyridine-2-carbonyl)-azetidin-3-yl]-carbamic acid tert-butyl ester (105 mg, 0.23 mmol) and reaction was sonicated. After 2 hours the precipitate was filtered to give (3-amino-azetidin-1-yl)-(6-(1H-pyrazol-4-yl)-8-trifluoromethyl-imidazo[1,2-a]pyridin-2-yl)-methanone hydrochloride (100 mg, 100%) as a white solid. MS (ESI) m/z=351.0 (MH⁺).

### Step 3: N-[1-(6-(1H-pyrazol-4-yl)-8-trifluoromethyl-imidazo[1,2-a]pyridine-2-carbonyl)-azetidin-3-yl]-methanesulfonamide (compound 520)

Methanesulfonyl chloride (10 μL) was added to a solution of N,N-diisopropylethylamine (80 μL) and (3-amino-azetidin-1-yl)-(6-(1H-pyrazol-4-yl)-8-trifluoromethyl-imidazo[1,2-a]pyridin-2-yl)-methanone (50 mg, 0.13 mmol) in DMF (300 μL). After 2 hours, water was added and the precipitate was filtered and subjected to silica chromatography to give N-[1-(6-(1H-pyrazol-4-yl)-8-trifluoromethyl-imidazo[1,2-a]pyridine-2-carbonyl)-azetidin-3-yl]-methanesulfonamide (compound 520, 21 mg, 38%) as a white solid. ¹H NMR (d₆-DMSO, 300 MHz) [symbol] 2.95 (s, 3H), 3.94 (dd, 1H, J=4.7, 10.0 Hz), 4.27-4.50 (m, 3H), 4.93 (dd, 1H, J=8.2, 9.7 Hz), 7.91 (d, 1H, J=7.9 Hz), 8.03 (s, 1H), 8.09 (s, 1H), 8.40 (s, 1H), 8.41 (s, 1H), 9.12 (s, 1H), 13.14 (s, 1H); MS (ESI) m/z=429.0 (MH⁺).

### Example 421

### N-{1-[3-Chloro-6-(1H-pyrazol-4-yl)-8-trifluoromethyl-imidazo[1,2-a]pyridine-2-carbonyl]-azetidin-3-yl}-methanesulfonamide (Compound 521)

### Step 1: 3-Chloro-6-(1H-pyrazol-4-yl)-8-trifluoromethyl-imidazo[1,2-a]pyridine-2-carboxylic Acid

N-chlorosuccinimide (1.78 g, 13.4 mmol) was added to a suspension of 6-(1-tert-butoxycarbonyl-1H-pyrazol-4-yl)-8-trifluoromethyl-imidazo[1,2-a]pyridine-2-carboxylic acid methyl ester (5 g, 12.2 mmol) in DMF (61 mL) at room temperature. The reaction was heated to 50° C. for 4 hours and then cooled to room temperature. After 18 hours, the reaction was quenched with 5% aqueous NaHSO₃. The precipitate was filtered and washed successively with H₂O and diethyl ether, and then air dried to obtain 6-(1-tert-butoxycarbonyl-1H-pyrazol-4-yl)-3-chloro-8-trifluoromethyl-imidazo[1,2-a]pyridine-2-carboxylic acid methyl ester (4.73 g, 87%) as a beige solid. MS (ESI) m/z=445.0 (MH⁺).

### Step 2: 3-Chloro-6-(1H-pyrazol-4-yl)-8-trifluoromethyl-imidazo[1,2-a]pyridine-2-carboxylic Acid

An aqueous solution of NaOH (1 M, 43 mL) was added slowly to a solution of 6-(1-tert-butoxycarbonyl-1H-pyrazol-4-yl)-3-chloro-8-trifluoromethyl-imidazo[1,2-a]pyridine-2-carboxylic acid methyl ester (4.732 g, 10.65 mmol) in THF and DMF (5:1 v/v, 146 mL) at room temperature. After 4 hours the pH was adjusted to 4 with aqueous citric acid (1 M). The residual THF was removed and the resulting precipitate was filtered and washed successively with H₂O and diethyl ether, and then air dried to obtain 3-chloro-6-(1H-pyrazol-4-yl)-8-trifluoromethyl-imidazo[1,2-a]pyridine-2-carboxylic acid (3.04 g, 87%) as a beige solid. MS (ESI) m/z=331.0 (MH⁺).

### Step 3: {1-[3-Chloro-6-(1H-pyrazol-4-yl)-8-trifluoromethyl-imidazo[1,2-a]pyridine-2-carbonyl]-azetidin-3-yl}-carbamic Acid Tert-butyl Ester

Prepared using standard HATU coupling conditions with the above acid. MS (ESI) m/z=485.1 (MH⁺).

### Step 4: (3-Amino-azetidin-1-yl)-(3-chloro-6-(1H-pyrazol-4-yl)-8-trifluoromethyl-imidazo[1,2-a]pyridin-2-yl)-methanone Hydrochloride

A solution of hydrogen chloride in dioxane (4M, 2 mL) was added to [1-(3-chloro-6-(1H-pyrazol-4-yl)-8-trifluoromethyl-imidazo[1,2-a]pyridine-2-carbonyl)-azetidin-3-yl]-carbamic acid tert-butyl ester (101 mg, 0.208 mmol) and reaction was sonicated. After 2 hours the precipitate was filtered to give (3-amino-azetidin-1-yl)-(3-chloro-6-(1H-pyrazol-4-yl)-8-trifluoromethyl-imidazo[1,2-a]pyridin-2-yl)-methanone hydrochloride (90, 100%) as a white solid. MS (ESI) m/z=385.0 (MH⁺).

### Step 5: N-{1-[3-Chloro-6-(1H-pyrazol-4-yl)-8-trifluoromethyl-imidazo[1,2-a]pyridine-2-carbonyl]-azetidin-3-yl}-methanesulfonamide (Compound 521)

Methanesulfonyl chloride (9 μL) was added to a solution of N,N-diisopropylethylamine (80 L) and (3-amino-azetidin-1-yl)-(3-chloro-6-(1H-pyrazol-4-yl)-8-trifluoromethyl-imidazo[1,2-a]pyridin-2-yl)-methanone (50 mg, 0.119 mmol) in DMF (600 μL). After 2 hours, water was added and the precipitate was filtered and subjected to silica chromatography to give N-[1-(3-chloro-6-(1H-pyrazol-4-yl)-8-trifluoromethyl-imidazo[1,2-a]pyridine-2-carbonyl)-azetidin-3-yl]-methanesulfonamide (20 mg, 36%) as a white solid. 1H NMR (d₆-DMSO, 300 MHz) [symbol] 2.94 (s, 3H), 3.95 (dd, 1H, J=5.0, 10.0 Hz), 4.28-4.46 (m, 3H), 4.86 (dd, 1H, J=7.3, 9.4 Hz), 7.91 (d, 1H, J=8.2 Hz), 8.21 (s, 1H), 8.25 (s, 1H), 8.56 (s, 1H), 8.83 (s, 1H), 13.16 (s, 1H); MS (ESI) m/z=463.0 (MH⁺).

### Example 422

### N-{1-[3-Brom o-6-(1H-pyrazol-4-yl)-8-trifluoromethyl-imidazo[1,2-a]pyridine-2-carbonyl]-azetidin-3-yl}-methanesulfonamide (Compound 522)

### Step 1: {1-[3-Bromo-6-(1H-pyrazol-4-yl)-8-trifluoromethyl-imidazo[1,2-a]pyridine-2-carbonyl]-azetidin-3-yl}-carbamic Acid Tert-butyl Ester

Using standard HATU coupling conditions, 3-bromo-6-(1H-pyrazol-4-yl)-8-trifluoromethyl-imidazo[1,2-a]pyridine-2-carboxylic acid (300 mg, 0.8 mmol) and 3-N-Boc-amino-azetidine (167 mg, 0.8 mmol) gave [1-(3-bromo-6-(1H-pyrazol-4-yl)-8-trifluoromethyl-imidazo[1,2-a]pyridine-2-carbonyl)-azetidin-3-yl]-carbamic acid tert-butyl ester (182 mg, 43%) as a beige solid. MS (ESI) m/z=529.0 (MH⁺).

### Step 2: (3-Amino-azetidin-1-yl)-(3-Bromo-6-(1H-pyrazol-4-yl)-8-trifluoromethyl-imidazo[1,2-a]pyridin-2-yl)-methanone Hydrochloride

A solution of hydrogen chloride in dioxane (4M, 3 mL) was added to [1-(3-bromo-6-(1H-pyrazol-4-yl)-8-trifluoromethyl-imidazo[1,2-a]pyridine-2-carbonyl)-azetidin-3-yl]-carbamic acid tert-butyl ester (182 mg, 0.344 mmol) and reaction was sonicated. After 2 hours, the precipitate was filtered to give (3-amino-azetidin-1-yl)-(3-bromo-6-(1H-pyrazol-4-yl)-8-trifluoromethyl-imidazo[1,2-a]pyridin-2-yl)-methanone hydrochloride (230 mg, 100%) as a white solid. MS (ESI) m/z=429.0 (MH⁺).

### Step 3: N-{1-[3-Bromo-6-(1H-pyrazol-4-yl)-8-trifluoromethyl-imidazo[1,2-a]pyridine-2-carbonyl]-azetidin-3-yl}-methanesulfonamide (Compound 522)

Methanesulfonyl chloride (8.4 μL) was added to a solution of N,N-diisopropylethylamine (80 μL) and (3-amino-azetidin-1-yl)-(3-bromo-6-(1H-pyrazol-4-yl)-8-trifluoromethyl-imidazo[1,2-a]pyridin-2-yl)-methanone (50 mg, 0.108 mmol) in DMF (300 μL). After 2 hours, water was added and the precipitate was filtered and subjected to silica chromatography to give N-[1-(3-bromo-6-(1H-pyrazol-4-yl)-8-trifluoromethyl-imidazo[1,2-a]pyridine-2-carbonyl)-azetidin-3-yl]-methanesulfonamide (16 mg, 29%) as a white solid. ¹H NMR (d₆-DMSO, 300 MHz) δ2.94 (s, 3H), 3.94 (dd, 1H, J=5.0, 10.3 Hz), 4.26-4.45 (m, 3H), 4.85 (dd, 1H, J=7.3, 10.3 Hz), 7.90 (d, 1H, J=8.2 Hz), 8.22 (s, 2H), 8.56 (s, 1H), 8.76 (s, 1H), 13.17 (s, 1H); MS (ESI) m/z=507.9 (MH⁺).

### Example 423

### (6-Bromo-3-chloro-8-trifluoromethyl-imidazo[1,2-a]pyridin-2-yl)-[3-(3-fluoro-phenyl)-pyrrolidin-1-yl]-methanone (Compound 523)

### Step 1: 5-Bromo-3-chloro-7-trifluoromethyl-imidazo[1,2-a]pyridine-2-carboxylic Acid

A mixture of 6-bromo-3-chloro-8-trifluoromethyl-imidazo[1,2-a]pyridine-2-carboxylic acid methyl ester (5 g, 13.99 mmol) and aqueous NaOH solution (2M, 20.98 mL, 41.96 mmol) in THF/H₂O (3:1 v/v, 100 mL) was stirred at room temperature for 2 hours. The mixture was concentrated and the residue was acidified with 10% HCl and extracted with DCM (2×80 mL). The organic layer was washed with brine (50 mL), dried (MgSO₄), and the filtrate was concentrated to afford 5-bromo-3-chloro-7-trifluoromethyl-imidazo[1,2-a]pyridine-2-carboxylic acid as a light yellow powder (4.42 g, 92%). ¹H NMR (d₆-DMSO, 300 MHz) δ 13.5 (s, 1H), 8.98 (d, 1H, J=0.8 Hz), 8.09 (s, 1H). MS (ESI) m/z=345 (MH⁺).

### Step 2: (6-Bromo-3-chloro-8-trifluoromethyl-imidazo[1,2-a]pyridin-2-yl)-[3-(3-fluoro-phenyl)-pyrrolidin-1-yl]-methanone (Compound 523)

A solution of 5-bromo-3-chloro-7-trifluoromethyl-imidazo[1,2-a]pyridine-2-carboxylic acid (852 mg, 2.48 mmol), HATU (1.41 g, 3.72 mmol), N,N-diisopropylethylamine (1.30 mL, 7.44 mmol), and 3-(3-fluoro-phenyl)-pyrrolidine HCl salt (1.00 g, 4.96 mmol) in DMF (10 mL) was stirred at 55° C. for 1.5 hours. The mixture was taken up in EtOAc (50 mL) and washed with H₂O (30 mL), saturated aqueous NaHCO₃ (30 mL), brine (30 mL), dried (MgSO₄), the filtrate was concentrated on silica and subjected to flash column chromatography [EtOAc/n-hexane (2:3 v/v)] to afford (6-bromo-3-chloro-8-trifluoromethyl-imidazo[1,2-a]pyridin-2-yl)-[3-(3-fluoro-phenyl)-pyrrolidin-1-yl]-methanone (1.05 g, 86%) as a white solid. ¹H NMR (d₆-DMSO, 300 MHz) δ 8.97 (m, 1H), 8.04 (m, 1H), 7.33 (m, 1H), 7.14 (m, 3H), 4.20 (m, 0.5H), 4.01 (m, 1H), 3.60 (m, 3.5H), 2.30 (m, 1H), 2.07 (m, 1H). MS (ESI) m/z=492.0 (MH⁺).

### Example 424

### [3-Chloro-6-(1H-pyrrol-3-yl)-8-trifluoromethyl-imidazo[1,2-a]pyridin-2-yl]-[3-(3-fluoro-phenyl)-pyrrolidin-1-yl]methanone (Compound 524)

A mixture of (6-bromo-3-chloro-8-trifluoromethyl-imidazo[1,2-a]pyridin-2-yl)-[3-(3-fluoro-phenyl)-pyrrolidin-1-yl]-methanone (Compound 523 in Example 423, 100 mg, 0.20 mmol), 1-(triisopropylsilyl)pyrrole-3-boronic acid (81.4 mg, 0.31 mmol), and Pd(PPh₃)₄ (12 mg, 0.01 mmol) in 3M K₃PO₄ (0.68 mL, 2.04 mmol) and 1,4-dioxane (2 mL) was stirred at 90° C. overnight. A solution of K₂CO₃ (85 mg, 0.612 mmol) in H₂O (2 mL) was added and the mixture stirred at 90° C. overnight. The mixture was diluted with EtOAc (20 mL), washed with saturated aqueous NaHCO₃ (10 mL), brine (10 mL), dried (MgSO₄), and the filtrate was concentrated. Preparative TLC (10% MeOH/DCM) afforded [3-chloro-6-(1H-pyrrol-3-yl)-8-trifluoromethyl-imidazo[1,2-a]pyridin-2-yl]-[3-(3-fluoro-phenyl)-pyrrolidin-1-yl]methanone (53 mg, 55%) as light brown solid. ¹H NMR (d₆-DMSO, 300 MHz) [symbol] 11.17 (s, 1H), 8.58 (d, J=0.90 Hz, 1H), 8.10 (d, J=7.80 Hz, 1H), 7.59 (m, 1H), 7.36 (m, 1H), 7.18 (m, 2H), 7.07 (m, 1H), 6.88 (m, 1H), 6.69 (m, 1H), 4.27 (m, 1H), 4.07 (m, 1H), 3.79 (m, 1.5H), 3.50 (m, 1.5H), 2.30 (m, 1H), 2.06 (m, 1H). MS (ESI) m/z=477.1 (MH⁺).

### Example 425

### [3-Chloro-6-(1H-indol-3-yl)-8-trifluoromethyl-imidazo[1,2-a]pyridin-2-yl]-[3-(3-fluoro-phenyl)-pyrrolidin-1-yl]methanone (Compound 525)

Prepared using similar procedure as in Example 424 (compound 524)

¹H NMR (d₆-DMSO, 300 MHz) [symbol] 11.69 (s, 1H), 8.66 (d, J=4.80 Hz, 1H), 8.20 (d, J=7.80 Hz, 1H), 8.09 (m, 1H), 7.88 (m, 1H), 7.50 (m, 1H), 7.36 (m, 1H), 7.18 (m, 4H), 7.11 (m, 1H), 4.30 (m, 1H), 4.11 (m, 1H), 3.84 (m, 1.5H), 3.60 (m, 1.5H), 2.32 (m, 1H), 2.12 (m, 1H). MS (ESI) m/z=527.1 (MH⁺).

### Example 426

### (3-Chloro-6-furan-3-yl-8-trifluoromethyl-imidazo[1,2-a]pyridine-2-yl)-(3-hydroxy-pyrrolidin-1-yl)-methanone (Compound 526)

Prepared using standard HATU coupling. ¹H NMR (d₆-DMSO, 300 MHz) δ 8.80 (s, 1H), 8.54 (s, 1H), 8.18 (s, 1H), 7.82 (m, 1H), 7.31 (m, 1H), 5.00 (dd, 1H, J=3.00, 9.00 Hz), 4.32 (m, 1H), 3.85 (m, 1.5H), 3.58 (m, 2.5H), 1.93 (m, 1H), 1.85 (m, 1H). MS (ESI) m/z=400.1 (MH⁺).

### Example 427

### (3-Chloro-6-furan-3-yl-8-trifluoromethyl-imidazo[1,2-a]pyridine-2-yl)-(3-(R)-hydroxy-pyrrolidin-1-yl)-methanone (Compound 527)

Prepared using standard HATU coupling. ¹H NMR (d₆-DMSO, 300 MHz) δ 8.80 (s, 1H), 8.54 (s, 1H), 8.18 (s, 1H), 7.83 (m, 1H), 7.31 (m, 1H), 5.00 (dd, 1H, J=3.30, 8.40 Hz), 4.32 (m, 1H), 3.85 (m, 1.5H), 3.58 (m, 2.5H), 1.93 (m, 1H), 1.85 (m, 1H). MS (ESI) m/z=400.1 (MH⁺).

### Example 428

### (3-Chloro-6-furan-3-yl-8-trifluoromethyl-imidazo[1,2-a]pyridine-2-yl)-(2-phenyl-piperidin-1-yl)-methanone (Compound 528)

Prepared using standard HATU coupling. ¹H NMR (d₆-DMSO, 300 MHz) δ 8.82 (d, 2H, J=10.65 Hz), 8.57 (d, 1H, J=5.40 Hz), 8.20 (d, 1H, J=8.85 Hz), 7.86 (s, 1H), 7.39 (m, 6H), 5.94 (s, 0.5H), 5.49 (s, 0.5H), 4.49 (d, 0.5H, J=5.70 Hz), 3.97 (d, 0.5H, J=7.50 Hz), 2.99 (m, 1H), 2.66 (m, 1H), 1.96 (m, 1H), 1.58 (m, 4H). MS (ESI) m/z=474.1 (MH⁺).

### Example 429

### (3-Chloro-6-furan-3-yl-8-trifluoromethyl-imidazo[1,2-a]pyridine-2-yl)-[4-(2-fluoro-phenyl)-piperazin-1-yl]-methanone (Compound 529)

Prepared using standard HATU coupling. ¹H NMR (d₆-DMSO, 300 MHz) δ 8.81 (s, 1H), 8.55 (s, 1H), 8.20 (s, 1H), 7.83 (m, 1H), 7.32 (m, 1H), 7.01 (m, 4H), 3.83 (m, 4H), 3.18 (m, 2H), 3.11 (m, 2H). MS (ESI) m/z=493.1 (MH⁺).

### Example 430

### (3-Chloro-6-furan-3-yl-8-trifluoromethyl-imidazo[1,2-a]pyridine-2-yl)-[4-(4-fluoro-phenyl)-piperazin-1-yl]-methanone (Compound 530)

Prepared using standard HATU coupling. ¹H NMR (d₆-DMSO, 300 MHz) δ 8.81 (s, 1H), 8.55 (s, 1H), 8.20 (s, 1H), 7.83 (m, 1H), 7.32 (m, 1H), 7.01 (m, 4H), 3.84 (m, 4H), 3.11 (m, 2H), 3.03 (m, 2H). MS (ESI) m/z=493.1 (MH⁺).

### Example 431

### (3-Chloro-6-furan-3-yl-8-trifluoromethyl-imidazo[1,2-a]pyridine-2-yl)-[4-(3-fluoro-phenyl)-piperazin-1-yl]-methanone (Compound 531)

Prepared using standard HATU coupling. ¹H NMR (d₆-DMSO, 300 MHz) δ 8.82 (s, 1H), 8.55 (s, 1H), 8.20 (s, 1H), 7.83 (m, 1H), 7.32 (m, 1H), 7.24 (dd, J=7.80, 15.60 Hz, 1H), 6.80 (m, 1H), 6.77 (m, 1H), 6.57 (m, 1H), 3.82 (m, 4H), 3.31 (m, 2H), 3.22 (m, 2H). MS (ESI) m/z=493.1 (MH⁺).

### Example 432

### (3-Chloro-6-furan-3-yl-8-trifluoromethyl-imidazo[1,2-a]pyridine-2-yl)-[4-pyridin-2-yl-piperazin-1-yl]-methanone (Compound 532)

Prepared using standard HATU coupling. ¹H NMR (d₆-DMSO, 300 MHz) δ 8.82 (s, 1H), 8.55 (s, 1H), 8.20 (s, 1H), 8.12 (m, 1H), 7.83 (m, 1H), 7.32 (m, 1H), 6.87 (d, J=8.70 Hz, 1H), 6.67 (dd, J=4.80, 6.60 Hz, 1H), 3.78 (m, 4H), 3.62 (m, 2H), 3.54 (m, 2H). MS (ESI) m/z=476.1 (MH⁺).

### Example 433

### (3-Chloro-6-furan-3-yl-8-trifluoromethyl-imidazo[1,2-a]pyridine-2-yl)-[4-pyridin-4-yl-piperazin-1-yl]-methanone (Compound 533)

Prepared using standard HATU coupling. ¹H NMR (d₆-DMSO, 300 MHz) δ 8.82 (s, 1H), 8.55 (s, 1H), 8.20 (s, 1H), 8.17 (d, J=1.50 Hz, 1H), 8.15 (d, J=1.50 Hz, 1H), 7.83 (t, J=1.50 Hz, 1H), 7.32 (m, 1H), 6.85 (d, J=1.80 Hz, 1H), 6.83 (d, J=1.80 Hz, 1H), 3.85 (m, 2H), 3.80 (m, 2H), 3.46 (m, 2H), 3.39 (m, 2H). MS (ESI) m/z=476.1 (MH⁺).

### Example 434

### (3-Chloro-6-furan-3-yl-8-trifluoromethyl-imidazo[1,2-a]pyridine-2-yl)-(4-phenyl-piperazin-1-yl)-methanone (Compound 534)

Prepared using standard HATU coupling. ¹H NMR (d₆-DMSO, 300 MHz) δ 8.81 (s, 1H), 8.55 (s, 1H), 8.20 (s, 1H), 7.83 (t, J=1.20 Hz, 1H), 7.32 (m, 1H), 7.22 (m, 2H), 6.97 (d, J=7.80 Hz, 2H), 6.80 (t, J=6.90 Hz, 1H), 3.83 (m, 4H), 3.24 (m, 2H), 3.16 (m, 2H). MS (ESI) m/z=475.1 (MH⁺).

### Example 435

### (3-Chloro-6-furan-3-yl-8-trifluoromethyl-imidazo[1,2-a]pyridine-2-yl)-(4-phenyl-piperidin-1-yl)-methanone (Compound 535)

Prepared using standard HATU coupling. ¹H NMR (d₆-DMSO, 300 MHz) δ 8.80 (s, 1H), 8.54 (s, 1H), 8.17 (s, 1H), 7.82 (t, J=1.80 Hz, 1H), 7.26 (m, 6H), 4.67 (d, J=13.20 Hz, 1H), 4.18 (d, J=13.50 Hz, 1H), 3.23 (m, 1H), 2.88 (m, 2H), 1.92 (d, J=12.60 Hz, 1H), 1.78 (d, J=12.30 Hz, 1H), 1.63 (m, 2H). MS (ESI) m/z=474.1 (MH⁺).

### Example 436

### (3-Chloro-6-furan-3-yl-8-trifluoromethyl-imidazo[1,2-a]pyridine-2-yl)-(4-thiazol-2-yl-piperazin-1-yl)-methanone (Compound 536)

Prepared using standard HATU coupling. ¹H NMR (d₆-DMSO, 300 MHz) δ 8.81 (s, 1H), 8.55 (s, 1H), 8.20 (s, 1H), 7.83 (t, J=1.50 Hz, 1H), 7.32 (m, 1H), 7.18 (d, J=3.60 Hz, 1H), 6.88 (d, J=3.60 Hz, 1H), 3.83 (m, 4H), 3.52 (m, 2H), 3.46 (m, 2H). MS (ESI) m/z=482.0 (MH⁺).

### Example 437

### 3-Chloro-6-furan-3-yl-8-trifluoromethyl-imidazo[1,2-a]pyridine-2-yl)-(2,3,5,6-tetrahydro-[1,2′]bipyrazinyl-4-yl)-methanone (Compound 537)

Prepared using standard HATU coupling. ¹H NMR (d₆-DMSO, 300 MHz) δ 8.82 (s, 1H), 8.55 (s, 1H), 8.36 (d, J=1.50 Hz, 1H), 8.20 (s, 1H), 8.09 (m, 1H), 7.86 (d, J=2.70 Hz, 1H), 7.83 (t, J=1.80 Hz, 1H), 7.32 (m, 1H), 3.82 (m, 4H), 3.73 (m, 2H), 3.64 (m, 2H). MS (ESI) m/z=477.1 (MH⁺).

### Example 438

### 3-Chloro-6-furan-3-yl-8-trifluoromethyl-imidazo[1,2-a]pyridine-2-yl)-[4-(3,4-difluoro-phenyl)-piperazin-1-yl]-methanone (Compound 538)

Prepared using standard HATU coupling. ¹H NMR (d₆-DMSO, 300 MHz) δ 8.81 (s, 1H), 8.54 (s, 1H), 8.19 (s, 1H), 7.82 (t, J=1.50 Hz, 1H), 7.32 (m, 1H), 7.27 (dd, J=9.00, 19.50 Hz, 1H), 7.03 (dq, J=3.00 Hz, 1H), 6.78 (m, 1H), 3.82 (m, 4H), 3.23 (m, 2H), 3.16 (m, 2H). MS (ESI) m/z=511.1 (MH⁺).

### Example 439

### 3-Chloro-6-furan-3-yl-8-trifluoromethyl-imidazo[1,2-a]pyridine-2-yl)-[4-(4-trifluoromethyl-phenyl)-piperazin-1-yl]-methanone (Compound 539)

Prepared using standard HATU coupling. ¹H NMR (d₆-DMSO, 300 MHz) δ 8.82 (s, 1H), 8.55 (s, 1H), 8.20 (s, 1H), 7.83 (t, J=1.50 Hz, 1H), 7.51 (d, J=9.00 Hz, 2H), 7.32 (m, 1H), 7.09 (d, J=8.40 Hz, 2H), 3.87 (m, 4H), 3.42 (m, 2H), 3.34 (m, 2H). MS (ESI) m/z=543.1 (MH⁺).

### Example 440

### 2-[1-(3-Chloro-6-furan-3-yl-8-trifluoromethyl-imidazo[1,2-a]pyridine-2-carbonyl)-piperidin-4-yl]-benzonitrile (Compound 540)

Prepared using standard HATU coupling. ¹H NMR (d₆-DMSO, 300 MHz) δ 8.81 (s, 1H), 8.54 (s, 1H), 8.18 (s, 1H), 7.81 (m, 2H), 7.68 (m, 1H), 7.56 (d, J=8.10 Hz, 1H), 7.42 (t, J=7.20 Hz, 1H), 7.31 (m, 1H), 4.71 (d, J=12.90 Hz, 1H), 4.28 (d, J=12.90 Hz, 1H), 3.25 (m, 2H), 2.98 (t, J=11.40 Hz, 1H), 1.95 (d, J=11.10 Hz, 1H), 1.76 (m, 3H). MS (ESI) m/z=499.1 (MH⁺).

### Example 441

### (3-Chloro-6-furan-3-yl-8-trifluoromethyl-imidazo[1,2-a]pyridin-2-yl)-[4-(2-chloro-phenyl)-piperidin-1-yl]-methanone (Compound 541)

Prepared using standard HATU coupling. ¹H NMR (d₆-DMSO, 300 MHz) δ 8.80 (s, 1H), 8.54 (s, 1H), 8.18 (s, 1H), 7.83 (t, J=1.80 Hz, 1H), 7.41 (dt, J=8.10, 15.90 Hz, 2H), 7.32 (m, 2H), 7.26 (m, 1H), 4.70 (d, J=13.20 Hz, 1H), 4.23 (d, J=13.20 Hz, 1H), 3.25 (m, 2H), 2.96 (t, J=12.60 Hz, 1H), 1.92 (d, J=12.60 Hz, 1H), 1.78 (d, J=12.90 Hz, 1H), 1.67 (m, 2H). MS (ESI) m/z=508.1 (MH⁺).

### Example 442

### (3-Chloro-6-furan-3-yl-8-trifluoromethyl-imidazo[1,2-a]pyridin-2-yl)-(4-o-tolyl-piperidin-1-yl)-methanone (Compound 542)

Prepared using standard HATU coupling. ¹H NMR (d₆-DMSO, 300 MHz) δ 8.80 (s, 1H), 8.54 (s, 1H), 8.18 (s, 1H), 7.83 (t, J=1.50 Hz, 1H), 7.31 (m, 1H), 7.14 (m, 4H), 4.69 (d, J=13.50 Hz, 1H), 4.18 (d, J=12.90 Hz, 1H), 3.27 (m, 1H), 2.99 (m, 2H), 2.33 (s, 3H), 1.84 (d, J=12.30 Hz, 1H), 1.64 (m, 3H). MS (ESI) m/z=488.1 (MH⁺).

### Example 443

### (3-Chloro-6-furan-3-yl-8-trifluoromethyl-imidazo[1,2-a]pyridin-2-yl)-(4-pyridin-3-yl-piperazin-1-yl)-methanone (Compound 543)

Prepared using standard HATU coupling. ¹H NMR (d₆-DMSO, 300 MHz) δ 8.82 (s, 1H), 8.55 (s, 1H), 8.33 (d, J=2.40 Hz, 1H), 8.20 (s, 1H), 8.14 (d, J=3.90 Hz, 1H), 7.83 (t, J=1.80 Hz, 1H), 7.38 (m, 1H), 8.32 (m, 1H), 7.22 (m, 1H), 3.84 (m, 4H), 3.28 (m, 2H), 3.24 (m, 2H). MS (ESI) m/z=476.1 (MH⁺).

### Example 444

### (3-Chloro-6-furan-3-yl-8-trifluoromethyl-imidazo[1,2-a]pyridin-2-yl)-[2-(2-fluoro-phenyl)-piperidin-1-yl)-methanone (Compound 544)

Prepared using standard HATU coupling. ¹H NMR (d₆-DMSO, 300 MHz) δ 8.79 (s, 1H), 8.53 (s, 1H), 8.17 (s, 1H), 7.82 (s, 1H), 7.30 (m, 5H), 5.88 (s, 1H), 4.52 (m, 0.5H), 4.10 (m, 0.5H), 3.0 (m, 0.5H), 2.16 (m, 1H), 2.00 (m, 1H), 1.65 (m, 3.5H), 1.54 (m, 1H). MS (ESI) m/z=492.1 (MH⁺).

### Example 445

### (3-Chloro-6-furan-3-yl-8-trifluoromethyl-imidazo[1,2-a]pyridin-2-yl)-[2-(3-fluoro-phenyl)-piperidin-1-yl)-methanone (Compound 545)

Prepared using standard HATU coupling. ¹H NMR (d₆-DMSO, 300 MHz) δ 8.72 (d, J=19.20 Hz, 1H), 8.46 (d, J=10.20 Hz, 1H), 8.10 (d, J=18.90 Hz, 1H), 7.76 (s, 1H), 7.37 (m, 1H), 7.23 (d, J=10.50 Hz, 1H), 7.10 (m, 3H), 5.80 (s, 0.5H), 5.38 (s, 0.5H), 4.38 (d, J=13.20 Hz, 0.5H), 3.90 (d, J=22.50 Hz, 0.5H), 2.89 (m, 0.5H), 2.54 (m, 0.5H), 2.36 (m, 0.5H), 1.84 (m, 1H), 1.48 (m, 4.5H). MS (ESI) m/z=492.1 (MH⁺).

### Example 446

### (3-Chloro-6-furan-3-yl-8-trifluoromethyl-imidazo[1,2-a]pyridin-2-yl)-[2-(3-fluoro-phenyl)-piperidin-1-yl)-methanone (Compound 546)

Prepared using standard HATU coupling. ¹H NMR (d₆-DMSO, 300 MHz) δ 8.79 (d, J=14.40 Hz, 1H), 8.53 (d, J=7.50 Hz, 1H), 8.16 (d, J=16.50 Hz, 1H), 7.83 (s, 1H), 7.28 (m, 5H), 5.87 (s, 0.5H), 5.42 (s, 0.5H), 4.43 (d, J=10.80 Hz, 0.5H), 3.93 (d, J=12.30 Hz, 0.5H), 2.92 (m, 0.5H), 2.59 (m, 0.5H), 1.91 (m, 1H), 1.55 (m, 4.5H). MS (ESI) m/z=492.1 (MH⁺).

### Example 447

### (3-Chloro-6-furan-3-yl-8-trifluoromethyl-imidazo[1,2-a]pyridin-2-yl)-[3-(2-fluoro-phenyl)-piperidin-1-yl)-methanone (Compound 547)

Prepared using standard HATU coupling. ¹H NMR (d₆-DMSO, 300 MHz) δ 8.78 (d, J=13.80 Hz, 1H), 8.53 (d, J=10.20 Hz, 1H), 8.16 (d, J=18.30 Hz, 1H), 7.81 (m, 1H), 7.45 (t, J=7.50 Hz, 1H), 7.32 (m, 2H), 7.20 (m, 2H), 7.07 (m, 1H), 4.57 (t, J=12.30 Hz, 1H), 4.13 (d, J=12.90 Hz, 1H), 3.00 (m, 3H), 1.80 (m, 4H). MS (ESI) m/z=492.1 (MH⁺).

### Example 448

### (3-Chloro-6-furan-3-yl-8-trifluoromethyl-imidazo[1,2-a]pyridin-2-yl)-[3-(4-fluoro-phenyl)-piperidin-1-yl)-methanone (Compound 548)

Prepared using standard HATU coupling. ¹H NMR (d₆-DMSO, 300 MHz) δ 8.78 (d, J=11.40 Hz, 1H), 8.53 (d, J=6.60 Hz, 1H), 8.17 (d, J=9.00 Hz, 1H), 7.82 (m, 1H), 7.39 (m, 1H), 7.30 (m, 2H), 7.16 (t, J=8.70 Hz, 1H), 7.07 (t, J=9.00 Hz, 1H), 4.57 (dd, J=13.50, 21.90 Hz, 1H), 4.20 (d, J=11.40 Hz, 0.5H), 4.10 (d, J=13.50 Hz, 0.5H), 3.13 (m, 1H), 2.88 (m, 2H), 1.95 (m, 1H), 1.74 (m, 3H). MS (ESI) m/z=492.1 (MH⁺).

### Example 449

### (3-Chloro-6-furan-3-yl-8-trifluoromethyl-imidazo[1,2-a]pyridin-2-yl)-[3-(3-fluoro-phenyl)-piperidin-1-yl)-methanone (Compound 549)

Prepared using standard HATU coupling. ¹H NMR (d₆-DMSO, 300 MHz) δ 8.78 (d, J=11.10 Hz, 1H), 8.53 (d, J=6.00 Hz, 1H), 8.17 (d, J=8.40 Hz, 1H), 7.81 (m, 1H), 7.28 (m, 2H), 7.20 (m, 1H), 7.07 (m, 2H), 4.56 (t, J=12.00 Hz, 1H), 4.25 (d, J=12.30 Hz, 0.5H), 4.09 (d, J=12.30 Hz, 0.5H), 3.14 (m, 1H), 2.86 (m, 3H), 1.97 (m, 1H), 1.74 (m, 2H). MS (ESI) m/z=492.1 (MH⁺).

### Example 450

### (3-Chloro-6-furan-3-yl-8-trifluoromethyl-imidazo[1,2-a]pyridin-2-yl)-[4-(2-methoxy-phenyl)-piperidin-1-yl)-methanone (Compound 550)

Prepared using standard HATU coupling. ¹H NMR (d₆-DMSO, 300 MHz) δ 8.80 (s, 1H), 8.54 (s, 1H), 8.18 (s, 1H), 7.83 (t, J=1.80 Hz, 1H), 7.31 (m, 1H), 7.18 (m, 2H), 6.95 (m, 2H), 4.66 (d, J=12.30 Hz, 1H), 4.16 (d, J=13.20 Hz, 1H), 3.79 (s, 3H), 3.18 (m, 2H), 2.90 (m, 1H), 1.85 (m, 1H), 1.62 (m, 3H). MS (ESI) m/z=504.1 (MH⁺).

### Example 451

### (4-Benzo[d]isoxazol-3-yl-piperazin-1-yl)-(3-Chloro-6-furan-3-yl-8-trifluoromethyl-imidazo[1,2-a]pyridine-2-yl)-methanone (Compound 551)

Prepared using standard HATU coupling. ¹H NMR (d₆-DMSO, 300 MHz) δ 8.82 (s, 1H), 8.54 (d, J=1.20 Hz, 1H), 8.20 (t, J=1.20 Hz, 1H), 8.03 (d, 8.10 Hz, 1H), 7.82 (t, J=1.80 Hz, 1H), 7.59 (dd, J=0.60, 4.20 Hz, 2H), 7.31 (m, 2H), 3.90 (m, 4H), 3.60 (m, 2H), 3.51 (m, 2H). MS (ESI) m/z=516.0 (MH⁺).

### Example 452

### 1-[1-(3-Chloro-6-furan-3-yl-8-trifluoromethyl-imidazo[1,2-a]pyridine-2-carbonyl)-piperidin-4-yl]-1,3-dihydro-benzoimidazol-2-one (Compound 552)

Prepared using standard HATU coupling. ¹H NMR (d₆-DMSO, 300 MHz) δ 10.86 (s, 1H), 8.83 (s, 1H), 8.55 (s, 1H), 8.20 (s, 1H), 7.84 (t, J=1.50 Hz, 1H), 7.32 (d, J=1.20 Hz, 1H), 7.21 (d, J=4.20 Hz, 1H), 7.00 (m, 3H), 4.70 (d, J=11.40 Hz, 1H), 4.51 (t, J=13.80 Hz, 1H), 4.26 (d, J=12.30 Hz, 1H), 2.99 (t, J=10.50 Hz, 1H), 2.53 (m, 1H), 2.41 (m, 2H), 1.87 (d, J=9.00 Hz, 1H), 1.72 (d, J=9.30 Hz, 1H). MS (ESI) m/z=530.2 (MH⁺).

### Example 453

### 1-[1-(3-Chloro-6-furan-3-yl-8-trifluoromethyl-imidazo[1,2-a]pyridine-2-carbonyl)-piperidin-4-yl]-4-phenyl-1,3-dihydro-imidazol-2-one (Compound 553)

Prepared using standard HATU coupling. ¹H NMR (d₆-DMSO, 300 MHz) δ 10.71 (s, 1H), 8.83 (s, 1H), 8.56 (s, 1H), 8.20 (s, 1H), 7.84 (t, J=1.80 Hz, 1H), 7.37 (dd, J=1.20, 8.70 Hz, 2H), 7.32 (m, 4H), 7.17 (t, J=7.20 Hz, 1H), 4.68 (d, J=10.20 Hz, 1H), 4.26 (m, 2H), 3.01 (t, J=11.70 Hz, 1H), 2.54 (m, 1H), 1.91 (m, 4H). MS (ESI) m/z=556.2 (MH⁺).

### Example 454

### 3-Chloro-6-furan-3-yl-8-trifluoromethyl-imidazo[1,2-a]pyridine-2-carboxylic acid (2-pyridin-2-yl-ethyl)-amide (Compound 554)

Prepared using standard HATU coupling. ¹H NMR (d₆-DMSO, 300 MHz) δ 8.80 (s, 1H), 8.78 (s, 1H), 8.56 (s, 1H), 8.51 (t, J=6.00 Hz, 1H), 8.37 (m, 1H), 8.23 (d, J=1.50 Hz, 1H), 7.84 (m, 3H), 7.32 (m, 1H), 3.75 (m, 2H), 3.28 (m, 2H). MS (ESI) m/z=435.0 (MH⁺).

### Example 455

### (3-Chloro-6-furan-3-yl-8-trifluoromethyl-imidazo[1,2-a]pyridin-2-yl)-[4-(2-fluoro-phenyl)-3,6-dihydro-2H-pyridin-1-yl)-methanone (Compound 555)

### Step 1: 4-(2-Fluoro-phenyl)-3,6-dihydro-2H-pyridine-1-carboxylic Acid Tert-butyl Ester

A mixture of 3,6-dihydro-2H-pyridine-1-N-Boc-boronic acid pinacolato ester (209 mg, 0.68 mmol), 1-fluoro-2-iodobenzene (100 mg, 0.45 mmol), Pd(dppf)Cl₂*CH₂Cl₂ (22 mg, 0.03 mmol) in aqueous Na₂CO₃ (0.4 M, 1 mL) and ACN (1 mL) was degassed twice and stirred at 90° C. for 2 hours. The mixture was concentrated on silica and subjected to flash column chromatography [EtOAc/n-hexane (1:1 v/v)] to afford 4-(2-fluoro-phenyl)-3,6-dihydro-2H-pyridine-1-carboxylic acid tert-butyl ester (110 mg, 88%) as a pale yellow oil. ¹H NMR (d₆-DMSO, 300 MHz) δ 7.35 (m, 2H), 7.19 (m, 2H), 5.93 (s, 1H), 3.94 (m, 2H), 3.57 (t, J=6.00 Hz, 2H), 2.41 (m, 2H), 1.41 (s, 9H); MS (ESI) m/z=222.1 (MH⁺-^{t}Bu).

### Step 2: 4-(2-Fluoro-phenyl)-1,2,3,6-tetrahydro-pyridine hydrochloride

A solution of hydrogen chloride in 1,4-dioxane (4M, 1 mL) was added to a stirring solution of 4-(2-fluoro-phenyl)-3,6-dihydro-2H-pyridine-1-carboxylic acid tert-butyl ester (100 mg, 0.36 mmol) in 1,4-dioxane (1 mL) and the reaction mixture was stirred at room temperature overnight. The mixture was concentrated, and dried under vacuum overnight to afford 4-(2-fluoro-phenyl)-1,2,3,6-tetrahydro-pyridine hydrochloride (74 mg, 96%) as a light brown solid. MS (ESI) m/z=178.0 (MH⁺).

### Step 3: (3-Chloro-6-furan-3-yl-8-trifluoromethyl-imidazo[1,2-a]pyridin-2-yl)-[4-(2-fluoro-phenyl)-3,6-dihydro-2H-pyridin-1-yl)-methanone (compound 555)

Prepared using standard HATU coupling. ¹H NMR (d₆-DMSO, 300 MHz) [symbol] 8.81 (s, 1H), 8.54 (d, J=1.20 Hz, 1H), 8.19 (s, 1H), 7.83 (t, J=1.80 Hz, 1H), 7.38 (m, 1H), 7.31 (m, 2H), 7.19 (m, 2H), 6.09 (s, 0.5H), 5.95 (s, 0.5H), 4.34 (d, J=15.60 Hz, 2H), 3.85 (m, 2H), 2.56 (m, 2H). MS (ESI) m/z=490.1 (MH⁺).

### Example 456

### (3-Chloro-6-furan-3-yl-8-trifluoromethyl-imidazo[1,2-a]pyridin-2-yl)-(4-thiazol-2-yl-piperidin-1-yl)-methanone (Compound 556)

### Step 1: 4-Thiazol-2-yl-3,6-dihydro-2H-pyridine-1-carboxylic acid tert-butyl Ester

A mixture of 3,6-dihydro-2H-pyridine-1-N-Boc-boronic acid pinacolato ester (1.40 g, 4.53 mmol), 2-bromo-thiazole (619 mg, 3.77 mmol), Pd(dppf)Cl₂*CH₂Cl₂ (185 mg, 0.23 mmol) in aqueous Na₂CO₃ (2M, 5.66 mL, 11.32 mmol) and 1,4-dioxane (14 mL) was degassed twice and stirred at 90° C. for 2 hours. The mixture was concentrated on silica and subjected to flash column chromatography [EtOAc/n-hexane (1:1 v/v)] to afford 4-thiazol-2-yl-3,6-dihydro-2H-pyridine-1-carboxylic acid tert-butyl ester (655 mg, 65%) as pale yellow oil. ¹H NMR (CDCl₃, 300 MHz) [symbol] 7.76 (d, J=3.60 Hz, 1H), 7.22 d J=3.60 Hz, 1H), 6.56 (m, 1H), 4.11 (m, 2H), 3.64 (tJ=5.40 Hz, 2H), 2.70 (m, 2H), 1.50 (s, 9H); MS (ESI) m/z=267.1 (MH⁺-^{t} Bu).

### Step 2: 4-Thiazol-2-yl-piperidine-1-carboxylic Acid Tert-butyl Ester

A suspension of 4-thiazol-2-yl-3,6-dihydro-2H-pyridine-1-carboxylic acid tert-butyl ester (450 mg, 1.69 mmol) and Raney nickel (90 mg) in EtOH (10 mL) was hydrogenated under H₂ (g) at 65 psi. After 3 days, the mixture was filtered through Celite and the filtrate was concentrated to afford 4-Thiazol-2-yl-piperidine-1-carboxylic acid tert-butyl ester (400 mg, 93%) as pale yellow oil. ¹H NMR (CDCl₃, 300 MHz) δ7.71 (d, 1H, J=3.00 Hz), 7.22. d, 1H, J=3.30 Hz), 4.21 (m, 2H), 3.17 (m, 1H), 2.89 (m, 2H), 2.79 (m, 2H), 1.77 (m, 2H), 1.50 (s, 9H); MS (ESI) m/z=213 (MH⁺-^{t}Bu)

### Step 3: 4-Thiazol-2-yl-piperidine Hydrochloride

Prepared using similar procedure as in Example 455, Step 3. ¹H NMR (d₆-DMSO, 300 MHz) [symbol] 7.76 (d, 1H, J=3.00 Hz), 7.66 (d, 1H, J=3.30 Hz), 3.36 (m, 3H), 3.04 (m, 3H), 2.20 (m, 2H), 1.93 (m, 2H). MS (ESI) m/z=169 (MH⁺).

### Step 4: (3-Chloro-6-furan-3-yl-8-trifluoromethyl-imidazo[1,2-a]pyridin-2-yl)-(4-thiazol-2-yl-piperidin-1-yl)-methanone (Compound 556)

Prepared using standard HATU coupling. ¹H NMR (d₆-DMSO, 300 MHz) [symbol] 8.79 (s, 1H), 8.53 (s, 1H), 8.17 (s, 1H), 7.82 (m, 1H), 7.71 (d, 1H, J=3.60 Hz, 1H), 7.60 (d, 1H, J=3.00 Hz, 1H), 7.29 (m, 1H), 4.52 (m, 1H), 4.14 (m, 1H), 3.35 (m, 2H), 3.03 (m, 1H), 2.19 (m, 1H), 2.01 (m, 1H), 1.71 (m, 2H). MS (ESI) m/z=482 (MH⁺).

### Example 457

### (3-Chloro-6-furan-3-yl-8-trifluoromethyl-imidazo[1,2-a]pyridin-2-yl)-(4-thiazol-4-yl-piperidin-1-yl)-methanone (Compound 557)

### Step 1: 4-Thiazol-4-yl-3,6-dihydro-2H-pyridine-1-carboxylic Acid Tert-butyl Ester

Prepared using similar procedure as in Example 456, Step 1. ¹H NMR (d₆-DMSO, 300 MHz) δ 9.08 (s, 1H), 7.58 (s, 1H), 6.61 (s, 1H), 4.02 (t, J=2.10 Hz, 2H), 3.55 (m, 2H), 2.47 (m, 2H), 1.40 (s, 9H); MS (ESI) m/z=211 (MH⁺-^{t}Bu).

### Step 2: 4-Thiazol-4-yl-1,2,3,6-tetrahydro-pyridine Hydrochloride

Prepared using similar procedure as in Example 456, Step 3. ¹H NMR (d₆-DMSO, 300 MHz) δ 9.13 (s, 1H), 9.10 (s, 1H), 7.73 (s, 1H), 6.61 (m, 1H), 3.77 (m, 2H), 3.32 (m, 2H), 2.70 (m, 2H). MS (ESI) m/z=166.9 (MH⁺).

### Step 3: 4-Thiazol-4-yl-piperidine Hydrochloride

Prepared using similar procedure as in Example 456, Step 2. MS (ESI) m/z=169.0 (MH⁺).

### Step 4: (3-Chloro-6-furan-3-yl-8-trifluoromethyl-imidazo[1,2-a]pyridin-2-yl)-(4-thiazol-4-yl-piperidin-1-yl)-methanone (Compound 557)

Prepared using standard HATU coupling. ¹H NMR (d₆-DMSO, 300 MHz) δ 9.09 (d, J=1.80 Hz, 1H), 8.81 (s, 1H), 8.55 (s, 1H), 8.19 (s, 1H), 7.84 (m, 1H), 7.44 (d, J=2.10 Hz, 1H), 7.31 (m, 1H), 4.60 (d, J=13.20 Hz, 1H), 4.14 (d, J=13.50 Hz, 1H), 3.10 (m, 3H), 2.00 (m, 2H), 1.69 (m, 2H). MS (ESI) m/z=481.0 (MH⁺).

### Example 458

### (3-Chloro-6-furan-3-yl-8-trifluoromethyl-imidazo[1,2-a]pyridin-2-yl)-[4-(1H-imidazol-4-yl)-3,6-dihydro-2H-pyridin-1-yl]-methanone (Compound 558)

### Step 1: 4-Iodo-imidazole-1-sulfonic Acid Dimethylamide

N,N′-Dimethylsulfonamide chloride (550 μL, 5.16 mmol) was added to a stirring solution of 4-iodoimidazole (500 mg, 2.58 mmol) and triethylamine (0.90 mL, 6.44 mmol) in ACN (5 mL) at room temperature. After 2 hours, the mixture was concentrated on silica and subjected to flash column chromatography (10-40% EtOAc/n-hexane gradient) to afford 4-iodo-imidazole-1-sulfonic acid dimethylamide (620 mg, 80%) as a white solid. ¹H NMR (CDCl₃, 300 MHz) δ 6.23 (s, 1H), 5.78 (s, 1H), 1.34 (s, 6H). MS (ESI) m/z=301.9 (MH⁺).

### Step 2: 4-(1-Dim ethylsulfamoyl-1H-imidazol-4-yl)-3,6-dihydro-2H-pyridine-1-carboxylic Acid Tert-butyl Ester

Prepared using Suzuki coupling of the above iodide as in Example 455, Step 1. ¹H NMR (CDCl₃, 300 MHz) δ 7.85 (s, 1H), 7.09 (s, 1H), 6.48 (m, 1H), 4.85 (d, J=3.00 Hz, 2H), 3.62 (t, J=5.70 Hz, 2H), 2.40 (m, 2H), 1.55 (s, 6H), 1.46 (s, 9H); MS (ESI) m/z=357.1 (MH⁺-^{t}Bu)

### Step 3: 4-(1,2,3,6-Tetrahydro-pyridin-4-yl)-imidazole-1-sulfonic Acid Dimethyl Amide Hydrochloride

Prepared using similar procedure as in Example 455, Step 2. MS (ESI) m/z=257.0 (MH⁺).

### Step 4: 4-[1-(3-Chloro-6-furan-3-yl-8-trifluoromethyl-imidazo[1,2-a]pyridine-2-carbonyl)-1,2,3,6-tetrahydro-pyridin-4-yl]-imidazole-1-sulfonic Acid Dimethylamide

Prepared using standard HATU couopling. MS (ESI) m/z=569.1 (MH⁺).

### Step 5: (3-Chloro-6-furan-3-yl-8-trifluoromethyl-imidazo[1,2-a]pyridin-2-yl)-[4-(1H-imidazol-4-yl)-3,6-dihydro-2H-pyridin-1-yl]-methanone (compound 558)

Prepared using similar procedure as in Example 455, Step 2 with heating at 50° C. ¹H NMR (d₆-DMSO, 300 MHz) δ 9.15 (d, J=4.80 Hz, 1H), 8.83 (s, 1H), 8.57 (s, 1H), 8.22 (s, 1H), 7.85 (d, J=1.50 Hz, 1H), 7.77 (s, 1H), 7.33 (s, 1H), 6.57 (s, 0.5H), 6.45 (s, 0.5H), 4.41 (d, J=30.30 Hz, 2H), 3.89 (d, J=5.40 Hz, 2H), 2.55 (m, 2H). MS (ESI) m/z=462.0 (MH⁺).

### Example 459

### (3-Chloro-6-furan-3-yl-8-trifluoromethyl-imidazo[1,2-a]pyridine-2-yl)-(4-thiazol-2-yl-3,6-dihydro-2H-pyridin-1-yl)-methanone (Compound 559)

### Step 1: 4-(4,4,5,5-Tetramethyl-[1,3,2]dioxaborolan-2-yl)-1,2,3,6-tetrahydro-pyridine Hydrochloride

4-(4,4,5,5-Tetramethyl-[1,3,2]dioxaborolan-2-yl)-3,6-dihydro-2H-pyridine-1-carboxylic acid tert-butyl ester underwent HCl deprotection to give 4-(4,4,5,5-tetramethyl-[1,3,2]dioxaborolan-2-yl)-1,2,3,6-tetrahydro-pyridine hydrochloride. ¹H NMR (d₆-DMSO, 300 MHz) δ 8.90 (s, 1H), 6.36 (m, 1H), 3.60 (m, 2H), 3.10 (m, 2H), 2.27 (m, 2H), 1.22 (s, 12H). MS (ESI) m/z=209.8 (MH⁺).

### Step 2: (3-Chloro-6-furan-3-yl-8-trifluoromethyl-imidazo[1,2-a]pyridin-2-yl)-[4-(4,4,5,5-tetramethyl-[1,3,2]dioxaborolan-2-yl)-3,6-dihydro-2H-pyridin-1-yl]-methanone

Prepared using standard HATU coupling with amine prepared in Step 1. ¹H NMR (d₆-DMSO, 300 MHz) δ 8.80 (s, 1H), 8.54 (s, 1H), 8.18 (s, 1H), 7.83 (t, J=1.80 Hz, 1H) 7.30 (m, 1H), 6.49 (s, 0.5H), 6.31 (s, 0.5H), 4.21 (d, J=8.10 Hz, 2H), 3.70 (m, 1H), 3.61 (m, 1H), 2.21 (s, 2H), 1.20 (s, 12H). MS (ESI) m/z=522.1 (MH⁺).

### Step 3: (3-Chloro-6-furan-3-yl-8-trifluoromethyl-imidazo[1,2-a]pyridine-2-yl)-(4-thiazol-2-yl-3,6-dihydro-2H-pyridin-1-yl)-methanone (Compound 559)

Prepared using Suzuki coupling as in Example 456, Step 1. ¹H NMR (d₆-DMSO, 300 MHz) δ 8.83 (s, 1H), 8.56 (s, 1H), 8.22 (s, 1H), 7.84 (s, 1H), 7.82 (d, J=3.30 Hz, 2H), 7.68 (m, 1H), 7.33 (m, 1H), 6.72 (s, 0.5H), 6.57 (s, 0.5H), 4.41 (d, J=19.50 Hz, 2H), 3.85 (m, 2H), 2.73 (m, 2H). MS (ESI) m/z=480 (MH⁺).

### Example 460

### 2-[1-(3-Chloro-6-furan-3-yl-8-trifluoromethyl-imidazo[1,2-a]pyridine-2-carbonyl)-1,2,3,6-tetrahydro-pyridin-4-yl]-N,N-diethyl-benzamide (Compound 560)

Prepared using similar procedure as in Example 459 (compound 559). ¹H NMR (d₆-DMSO, 300 MHz) δ 8.81 (s, 1H), 8.55 (s, 1H), 8.19 (s, 1H), 7.83 (t, J=1.80 Hz, 1H), 7.34 (m, 4H), 7.19 (m, 1H), 5.80 (s, 0.5H), 5.67 (s, 0.5H), 4.25 (d, J=26.10 Hz, 2H), 3.79 (m, 2H), 3.00 (m, 4H), 2.71 (m, 0.5H), 2.56 (m, 0.5H), 2.26 (m, 1H), 1.07 (m, 2H), 0.95 (m, 4H). MS (ESI) m/z=571.1 (MH⁺).

### Example 461

### (3-Chloro-6-furan-3-yl-8-trifluoromethyl-imidazo[1,2-a]pyridin-2-yl)-[4-(2-hydroxymethyl-phenyl)-3,6-dihydro-2H-pyridin-1-yl)-methanone (Compound 561)

Prepared using similar procedure as in Example 459 (compound 559). ¹H NMR (d₆-DMSO, 300 MHz) δ 8.77 (s, 1H), 8.49 (s, 1H), 8.14 (s, 1H), 7.78 (t, J=1.80 Hz, 1H), 7.41 (d, J=7.20 Hz, 1H), 7.27 (s, 1H), 7.19 (m, 2H), 7.07 (d, J=7.20 Hz, 1H), 5.64 (s, 0.5H), 5.50 (s, 0.5H), 5.04 (m, 1H), 4.42 (t, J=7.50 Hz, 2H), 4.24 (d, J=6.30 Hz, 2H), 3.80 (m, 2H), 2.37 (m, 2H). MS (ESI) m/z=502.1 (MH⁺).

### Example 462

### (3-Chloro-6-furan-3-yl-8-trifluoromethyl-imidazo[1,2-a]pyridin-2-yl)-[4-(2,6-dimethoxy-phenyl)-3,6-dihydro-2H-pyridin-1-yl)-methanone (Compound 562)

Prepared using similar procedure as in Example 459 (compound 559). ¹H NMR (d₆-DMSO, 300 MHz) δ 8.76 (s, 1H), 8.49 (s, 1H), 8.14 (s, 1H), 7.78 (t, J=1.50 Hz, 1H), 7.26 (s, 1H), 7.13 (m, 1H), 6.60 (d, J=3.00 Hz, 1H), 6.57 (d, J=3.00 Hz, 1H), 5.48 (s, 0.5H), 5.34 (s, 0.5H), 4.18 (m, 2H), 3.81 (m, 1H), 3.72 (m, 1H), 3.70 (s, 3H), 3.65 (s, 3H), 2.21 (m, 2H). MS (ESI) m/z=532.1 (MH⁺).

### Example 463

### 2-[1-(3-Chloro-6-furan-3-yl-8-trifluoromethyl-imidazo[1,2-a]pyridine-2-carbonyl)-1,2,3,6-tetrahydro-pyridin-4-yl]-benzonitrile (Compound 563)

Prepared using similar procedure as in Example 459 (compound 559). ¹H NMR (d₆-DMSO, 300 MHz) δ 8.84 (s, 1H), 8.56 (s, 1H), 8.21 (s, 1H), 7.85 (m, 2H), 7.71 (t, J=8.10 Hz; 1H), 7.53 (m, 2H), 7.33 (m, 1H), 6.13 (s, 0.5H), 6.00 (s, 0.5H), 4.39 (d, J=20.40 Hz, 2H), 3.92 (m, 2H), 2.63 (m, 2H). MS (ESI) m/z=497.0 (MH⁺).

### Example 464

### (3-Chloro-6-furan-3-yl-8-trifluoromethyl-imidazo[1,2-a]pyridin-2-yl)-[4-(2,6-difluoro-phenyl)-3,6-dihydro-2H-pyridin-1-yl)-methanone (Compound 564)

Prepared using similar procedure as in Example 459 (compound 559). ¹H NMR (d₆-DMSO, 300 MHz) δ 8.77 (s, 1H), 8.49 (s, 1H), 8.14 (s, 1H), 7.78 (t, J=1.80 Hz, 1H), 7.33 (m, 1H), 7.26 (m, 1H), 7.07 (m, 2H), 5.92 (s, 0.5H), 5.77 (s, 0.5H), 4.30 (d, J=17.70 Hz, 2H), 3.80 (m, 2H), 2.47 (m, 2H). MS (ESI) m/z=508.0 (MH⁺).

### Example 465

### 2-[1-(3-Chloro-6-furan-3-yl-8-trifluoromethyl-imidazo[1,2-a]pyridine-2-carbonyl)-1,2,3,6-tetrahydro-pyridin-4-yl]-3-fluoro-benzonitrile (Compound 565)

Prepared using similar procedure as in Example 459 (compound 559). ¹H NMR (d₆-DMSO, 300 MHz) δ 8.77 (s, 1H), 8.49 (s, 1H), 8.14 (s, 1H), 7.78 (t, J=1.50 Hz, 1H), 7.68 (m, 1H), 7.54 (m, 2H), 7.26 (m, 1H), 6.04 (s, 0.5H), 5.90 (s, 0.5H), 4.33 (d, J=18.90 Hz, 2H), 3.84 (m, 2H), 2.51 (m, 2H). MS (ESI) m/z=515.0 (MH⁺).

### Example 466

### (3-Chloro-6-furan-3-yl-8-trifluoromethyl-imidazo[1,2-a]pyridin-2-yl)-(4-thiazol-4-yl-3,6-dihydro-2H-pyridin-1-yl-methanone (Compound 566)

Prepared using similar procedure as in Example 459 (compound 559). ¹H NMR (d₆-DMSO, 300 MHz) δ 8.98 (d, 1H, J=5.10 Hz), 8.83 (s, 1H), 8.56 (s, 1H), 8.21 (s, 11H), 7.92 (m, 1H), 7.84 (m, 1H), 7.33 (s, 1H), 6.26 (s, 0.5H), 6.11 (s, 0.5H), 4.36 (d, J=21.60 Hz, 2H), 3.88 (m, 2H), 2.63 (m, 2H). MS (ESI) m/z=479.9 (MH⁺).

### Example 467

### (3-Chloro-6-furan-3-yl-8-trifluoromethyl-imidazo[1,2-a]pyridin-2-yl)-[4-(2-ethynyl-phenyl)-3,6-dihydro-2H-pyridin-1-yl)-methanone (Compound 567)

Prepared using similar procedure as in Example 459 (compound 559). ¹H NMR (CDCl₃, 300 MHz) δ 8.33 (s, 1H), 7.85 (s, 1H), 7.76 (s, 1H), 7.59 (t, J=1.50 Hz, 1H), 7.52 (m, 2H), 7.28 (m, 2H), 6.75 (m, 1H), 5.92 (s, 0.5H), 5.81 (s, 0.5H), 4.59 (d, J=2.40 Hz, 1H), 4.44 (d, J=2.70 Hz, 1H), 4.05 (m, 2H), 2.76 (m, 2H), 1.26 (m, 1H). MS (ESI) m/z=496.0 (MH⁺).

### Example 468

### (3-Chloro-6-furan-3-yl-8-trifluoromethyl-imidazo[1,2-a]pyridin-2-yl)-(4-thiazol-5-yl-3,6-dihydro-2H-pyridin-1-yl-methanone (Compound 568)

Prepared using similar procedure as in Example 459 (compound 559). ¹H NMR (d₆-DMSO, 300 MHz) δ 8.95 (d, 1H, J=5.10 Hz), 8.81 (s, 1H), 8.54 (s, 1H), 8.19 (s, 1H), 7.90 (d, 1H, J=6.00 Hz), 7.83 (s, 1H), 7.31 (s, 1H), 6.24 (s, 0.5H), 6.09 (s, 0.5H), 4.34 (d, J=21.60 Hz, 2H), 3.88 (m, 2H), 2.54 (m, 2H). MS (ESI) m/z=480 (MH⁺).

### Example 469

### 2-[1-(3-Chloro-6-furan-3-yl-8-trifluoromethyl-imidazo[1,2-a]pyridine-2-carbonyl)-1,2,3,6-tetrahydro-pyridin-4-yl]-4-fluoro-benzonitrile (Compound 569)

Prepared using similar procedure as in Example 459 (compound 559). ¹H NMR (d₆-DMSO, 300 MHz) δ 8.84 (s, 1H), 8.56 (s, 1H), 8.21 (s, 1H), 7.98 (m, 1H), 7.84 (t, J=1.80 Hz, 1H), 7.48 (m, 1H), 7.38 (m, 2H), 6.21 (s, 0.5H), 6.08 (s, 0.5H), 4.40 (d, J=22.80 Hz, 2H), 3.92 (m, 2H), 2.63 (m, 2H). MS (ESI) m/z=515.0 (MH⁺).

### Example 470

### 2-[1-(3-Chloro-6-furan-3-yl-8-trifluoromethyl-imidazo[1,2-a]pyridine-2-carbonyl)-1,2,3,6-tetrahydro-pyridin-4-yl]-5-fluoro-benzonitrile (Compound 570)

Prepared using similar procedure as in Example 459 (compound 559). ¹H NMR (d₆-DMSO, 300 MHz) δ 8.84 (s, 1H), 8.84 (s, 1H), 8.56 (s, 1H), 8.21 (s, 1H), 7.88 (m, 1H), 7.84 (t, J=2.10 Hz, 1H), 7.60 (m, 2H), 7.33 (m, 1H), 6.12 (s, 0.5H), 5.99 (s, 0.5H), 4.39 (d, J=21.00 Hz, 2H), 3.90 (m, 2H), 2.61 (m, 2H). MS (ESI) m/z=515.0 (MH⁺).

### Example 471

### (3-Chloro-6-furan-3-yl-8-trifluoromethyl-imidazo[1,2-a]pyridin-2-yl)-(3-fluoro-3′,6′-dihydro-2′H-[2,4′]bipyridinyl-1′-yl)-methanone (Compound 571)

Prepared using similar procedure as in Example 459 (compound 559). ¹H NMR (d₆-DMSO, 300 MHz) δ 10.26 (s, 1H), 8.83 (s, 1H), 8.56 (s, 1H), 8.44 (m, 1H), 8.21 (s, 1H), 7.84 (t, J=1.80 Hz, 1H), 7.75 (m, 1H), 7.42 (m, 1H), 7.32 (m, 1H), 6.66 (s, 0.5H), 6.49 (s, 0.5H), 4.42 (d, J=20.70 Hz, 2H), 3.85 (m, 2H), 2.73 (m, 2H). MS (ESI) m/z=491.2 (MH⁺).

### Example 472

### (3-Chloro-6-furan-3-yl-8-trifluoromethyl-imidazo[1, 2-1]pyridine-2-yl)-(3′-fluoro-3,6-dihydro-2H-[4,4′]bipyridinyl-1-yl)-methanone (Compound 572)

Prepared using similar procedure as in Example 459 (compound 559). ¹H NMR (d₆-DMSO, 300 MHz) δ 8.84 (s, 1H), 8.63 (s, 1H), 8.56 (s, 1H), 8.46 (d, J=4.50 Hz, 1H), 8.21 (s, 1H), 7.84 (t, J=1.80 Hz, 1H), 7.54 (t, J=6.90 Hz, 1H), 7.33 (m, 1H), 6.46 (s, 0.5H), 6.33 (s, 0.5H), 4.43 (d, J=30.00 Hz, 2H), 3.90 (m, 2H), 2.62 (m, 2H). MS (ESI) m/z=491.0 (MH⁺).

### Example 473

### (3-Chloro-6-furan-3-yl-8-trifluoromethyl-imidazo[1,2-a]pyridine-2-yl)-[4-(5-hydroxymethyl-thiazol-2-yl)-3,6-dihydro-2H-pyridin-1-yl]-methanone (Compound 573)

Prepared using similar procedure as in Example 459 (compound 559). ¹H NMR (d₆-DMSO, 300 MHz) δ 8.83 (s, 1H), 8.56 (s, 1H), 8.21 (s, 1H), 7.84 (t, J=1.80 Hz, 1H), 7.62 (s, 1H), 7.33 (t, J=1.20 Hz, 1H), 6.65 (s, 0.5H), 6.50 (s, 0.5H), 4.65 (d, J=3.60 Hz, 2H), 4.40 (d, J=18.60 Hz, 2.5H), 3.86 (m, 2.5H), 2.70 (m, 2H). MS (ESI) m/z=508.9 (MH⁺).

### Example 474

### Trifluoro-methanesulfonic Acid 1-(3-chloro-6-furan-3-yl-8-trifluoromethyl-imidazo[1,2-a]pyridine-2-carbonyl)-1,2,3,6-tetrahydro-pyridin-4-yl Ester (Compound 574)

### Step 1: 4-Trifluoromethanesulfonyloxy-3,6-dihydro-2H-pyridine-1-carboxylic Acid Tert-butyl Ester

A solution of t-butoxycarbonyl-4-piperidone (3 g, 15.06 mmol) in THF (10 mL) was slowly added to a stirring 2M solution of LDA (9.03 mL, 18.07 mmol) in THF (10 mL) at −78° C. After 10 min, a solution of N-phenyl bis(trifluoromethanesulfonimide) (5.92 g, 16.56 mmol) in THF (10 mL) was slowly added. After 30 min, the cooling bath was removed and the mixture was allowed to warm to room temperature over the course of 1.5 hours. The mixture was cooled to 0° C., quenched with saturated aqueous NaHCO₃ (30 mL), and extracted with ether (200 mL). The organic layer was washed with 5% citric acid (40 mL), aqueous NaOH (1M, 4×40 mL), H₂O (2×40 mL), brine (40 mL), dried (MgSO₄), the filtrate was concentrated on silica and subjected to flash column chromatography (15-50% EtOAc/hexane gradient) to afford 4-trifluoromethanesulfonyloxy-3,6-dihydro-2H-pyridine-1-carboxylic acid tert-butyl ester (3.40 g, 68.2%) as a brown oil. ¹H NMR (CDCl₃, 300 MHz) δ 6.10 (t, J=3.30 Hz, 1H), 4.07 (m, 2H), 3.63 (t, J=5.70 Hz, 2H), 2.48 (m, 2H), 1.48 (s, 9H); MS (ESI) m/z=276 (MH⁺-^{t}Bu).

### Step 2: Trifluoro-methanesulfonic acid 1,2,3,6-tetrahydro-pyridin-4-yl-ester Hydrochloride

4-Trifluoromethanesulfonyloxy-3,6-dihydro-2H-pyridine-1-carboxylic acid tert-butyl ester underwent HCl deprotection to give trifluoro-methanesulfonic acid 1,2,3,6-tetrahydro-pyridin-4-yl-ester hydrochloride. ¹H NMR (CDCl₃, 300 MHz) δ 9.76 (s, 1H), 7.32 (m, 1H), 3.94 (m, 2H), 2.79 (m, 2H), 2.11 (m, 2H). MS (ESI) m/z=232.0 (MH⁺).

### Step 3: Trifluoro-methanesulfonic acid 1-(3-chloro-6-furan-3-yl-8-trifluoromethyl-imidazo[1,2-a]pyridine-2-carbonyl)-1,2,3,6-tetrahydro-pyridin-4-yl ester (Compound 574)

Prepared using standard HATU coupling of the above amine. ¹H NMR (d₆-DMSO, 300 MHz) δ 8.83 (s, 1H), 8.56 (s, 1H), 8.22 (s, 1H), 7.84 (t, J=2.10 Hz, 1H), 7.32 (m, 1H), 6.15 (s, 0.5H), 6.04 (s, 0.5H), 4.37 (d, J=31.20 Hz, 2H), 3.87 (m, 2H), 2.60 (m, 2H). MS (ESI) m/z=544 (MH⁺).

### Example 475

### (3-Chloro-6-furan-3-yl-8-trifluoromethyl-imidazo[1,2-a]pyridine-2-yl)-(4-furan-3-yl-3,6-dihydro-2H-pyridin-1-yl)-methanone (Compound 575)

Prepared using Suzuki reaction conditions as described in Example 455, Step 1 with the above triflate and 3-furanboronic acid. ¹H NMR (d₆-DMSO, 300 MHz) δ 8.80 (s, 1H), 8.54 (s, 1H), 8.18 (s, 1H), 7.82 (t, J=1.50 Hz, 1H), 7.74 (d, J=5.70 Hz, 1H), 7.62 (m, 1H), 7.30 (d, J=1.80 Hz, 1H), 6.72 (d, J=11.70 Hz, 1H), 6.10 (s, 0.5H), 5.95 (s, 0.5H), 4.30 (d, J=19.80 Hz, 2H), 3.82 (m, 2H), 2.42 (m, 2H). MS (ESI) m/z=461.9 (MH⁺).

### Example 476

### (3-Chloro-6-furan-3-yl-8-trifluoromethyl-imidazo[1,2-a]pyridin-2-yl)-[4-(3-fluoro-phenyl)-3,6-dihydro-2H-pyridin-1-yl)-methanone (Compound 576)

Prepared using similar procedure as in Example 475 (compound 575). ¹H NMR (d₆-DMSO, 300 MHz) δ 8.81 (s, 1H), 8.54 (s, 1H), 8.19 (s, 1H), 7.82 (t, J=1.80 Hz, 1H), 7.38 (m, 1H), 7.34 (m, 3H), 7.11 (m, 1H), 6.35 (s, 0.5H), 6.22 (s, 0.5H), 4.35 (d, J=18.00 Hz, 2H), 3.85 (m, 2H), 2.58 (m, 2H). MS (ESI) m/z=489.9 (MH⁺).

### Example 477

### 2-[1-(3-Chloro-6-furan-3-yl-8-trifluoromethyl-imidazo[1,2-a]pyridin-2-carbonyl)-1,2,3,6-tetrahydro-pyridin-4-yl)-N,N-dimethyl-benzenesulfonamide (Compound 577)

Prepared using similar procedure as in Example 475 (compound 575). ¹H NMR (d₆-DMSO, 300 MHz) δ 8.83 (s, 1H), 8.56 (s, 1H), 8.20 (s, 1H), 7.84 (m, 2H), 7.66 (m, 1H), 7.56 (m, 1H), 7.37 (m, 2H), 5.67 (s, 0.5H), 5.52 (s, 0.5H), 4.31 (d, J=13.20 Hz, 2H). 3.90 (m, 2H), 2.73 (s, 3H), 2.67 (s, 3H), 2.45 (m, 2H). MS (ESI) m/z=578.9 (MH⁺).

### Example 478

### (3-Chloro-6-furan-3-yl-8-trifluoromethyl-imidazo[1,2-a]pyridin-2-yl)-[4-(1-methyl-1H-pyrazol-4-yl)-3,6-dihydro-2H-pyridin-1-yl)-methanone (Compound 578)

Prepared using similar procedure as in Example 475 (compound 575). ¹H NMR (d₆-DMSO, 300 MHz) δ 8.80 (s, 1H), 8.54 (s, 1H), 8.12 (s, 1H), 7.82 (m, 1H), 7.76 (d, J=7.20 Hz, 1H), 7.55 (m, 1H), 7.31 (s, 1H), 6.00 (s, 0.5H), 5.86 (s, 0.5H), 4.25 (d, J=14.10 Hz, 2H), 3.84 (m, 2H), 3.77 (s, 3H), 2.42 (m, 2H). MS (ESI) m/z=476.2 (MH⁺).

### Example 479

### (3-Chloro-6-furan-3-yl-8-trifluoromethyl-imidazo[1,2-a]pyridin-2-yl)-[4-(1-1H-pyrazol-4-yl)-3,6-dihydro-2H-pyridin-1-yl)-methanone (Compound 579)

Prepared using similar procedure as in Example 475 (compound 575). ¹H NMR (d₆-DMSO, 300 MHz) δ 8.83 (s, 1H), 8.56 (s, 1H), 8.20 (s, 1H), 7.84 (m, 3H), 7.33 (s, 1H), 6.09 (s, 1H), 5.94 (s, 1H), 4.30 (d, J=17.10 Hz, 2H), 3.84 (m, 2H). MS (ESI) m/z=462.1 (MH⁺).

### Example 480

### (3-Chloro-6-furan-3-yl-8-trifluoromethyl-imidazo[1,2-a]pyridin-2-yl)-[4-(2-morpholin-4-yl-thiazol-4-yl)-3,6-dihydro-2H-pyridin-1-yl)-methanone (Compound 580)

Prepared using similar procedure as in Example 475 (compound 575). ¹H NMR (d₆-DMSO, 300 MHz) δ 8.83 (s, 1H), 8.56 (s, 1H), 8.21 (s, 1H), 7.84 (s, 1H), 7.33 (s, 1H), 6.79 (d, J=9.30 Hz, 1H), 6.55 (s, 0.5H), 6.38 (s, 0.5H), 4.34 (d, J=15.90 Hz, 2H), 3.82 (m, 2H), 3.71 (m, 4H), 3.38 (m, 4H), 2.47 (m, 2H). MS (ESI) m/z=563.0 (MH⁺).

### Example 481

### (3-Chloro-6-furan-3-yl-8-trifluoromethyl-imidazo[1,2-a]pyridin-2-yl)-(2-fluoro-3′,6′-dihydro-2′H-[3,4′]bipyridinyl-1′-yl)-methanone (Compound 581)

Prepared using similar procedure as in Example 475 (compound 575). ¹H NMR (d₆-DMSO, 300 MHz) δ 8.83 (s, 1H), 8.56 (m, 1H), 8.21 (s, 1H), 8.15 (m, 1H), 7.96 (m, 1H), 7.84 (t, J=1.80 Hz, 1H), 7.39 (m, 1H), 7.32 (m, 1H), 6.25 (s, 0.5H), 6.11 (s, 0.5H), 4.39 (d, J=20.40 Hz, 2H), 3.90 (m, 2H), 2.59 (m, 2H). MS (ESI) m/z=491.1 (MH⁺).

### Example 482

### (3-Chloro-6-furan-3-yl-8-trifluoromethyl-imidazo[1,2-a]pyridin-2-yl)-(4-isoxazol-4-yl-3,6-dihydro-2H-pyridin-1-yl)-methanone (Compound 582)

Prepared using similar procedure as in Example 475 (compound 575). ¹H NMR (d₆-DMSO, 300 MHz) δ 8.98 (s, 1H), 8.90 (d, J=12.00 Hz, 1H), 8.80 (s, 1H), 8.50 (s, 1H), 8.18 (s, 1H), 7.82 (t, J=1.50 Hz, 1H), 7.27 (d, J=1.20 Hz, 1H), 6.28 (s, 0.5H), 6.14 (s, 0.5H), 4.32 (d, J=19.80 Hz, 2H), 3.85 (m, 2H), 2.48 (m, 2H). MS (ESI) m/z=463.0 (MH⁺).

### Example 483

### (3-Chloro-6-furan-3-yl-8-trifluoromethyl-imid azo[1,2-a]pyridin-2-yl)-[4-(1H-pyrrol-3-yl)-3,6-dihydro-2H-pyridin-1-yl]-methanone (Compound 583)

Prepared using similar procedure as in Example 475 (compound 575). ¹H NMR (d₆-DMSO, 300 MHz) δ 8.83 (s, 1H), 8.56 (s, 1H), 8.20 (s, 1H), 7.84 (s, 1H), 7.33 (s, 1H), 6.83 (d, J=6.30 Hz, 1H), 6.72 (d, J=2.10 Hz, 1H), 6.21 (d, J=12.30 Hz, 1H), 5.89 (s, 0.5H), 5.74 (s, 0.5H), 4.26 (d, J=2.40 Hz, 2H), 3.78 (m, 2H), 2.46 (m, 2H). MS (ESI) m/z=461.1 (MH⁺).

### Example 484

### (3-Chloro-6-furan-3-yl-8-trifluoromethyl-imidazo[1,2-a]pyridin-2-yl)-[4-(2H-pyrazol-3-yl)-3,6-dihydro-2H-pyridin-1-yl]methanone (Compound 584)

Prepared using similar procedure as in Example 475 (compound 575). ¹H NMR (d₆-DMSO, 300 MHz) δ 8.83 (s, 1H), 8.83 (s, 1H), 8.56 (s, 1H), 8.21 (s, 1H), 7.95 (s, 1H), 7.84 (t, J=1.50 Hz, 1H), 7.71 (m, 1H), 7.33 (d, J=1.20 Hz, 1H), 6.49 (m, 1H), 6.38 (s, 0.5H), 6.24 (s, 0.5H), 4.35 (d, J=17.70 Hz, 2H), 3.84 (m, 2H), 2.60 (m, 2H). MS (ESI) m/z=462.0 (MH⁺).

### Example 485

### 1-{5-[1-(3-Chloro-6-furan-3-yl-8-trifluoromethyl-imidazo[1,2-a]pyridine-2-carbonyl)-1,2,3,6-tetrahydro-pyridin-4-yl]-thiophen-2-yl}-ethanone (Compound 585)

Prepared using similar procedure as in Example 475 (compound 575). ¹H NMR (d₆-DMSO, 300 MHz) δ 8.83 (s, 1H), 8.82 (s, 1H), 8.56 (s, 1H), 8.21 (s, 1H), 7.85 (m, 2H), 7.33 (d, J=2.70 Hz, 1H), 7.26 (d, J=3.90 Hz, 1H), 6.48 (s, 0.5H), 6.33 (s, 0.5H), 4.38 (d, J=23.10 Hz, 2H), 3.88 (m, 2H), 2.62 (m, 2H), 2.48 (s, 3H). MS (ESI) m/z=519.9 (MH⁺).

### Example 486

### (3-Chloro-6-furan-3-yl-8-trifluoromethyl-imidazo[1,2-a]pyridine-2-yl)-[4-(2-methyl-2H-pyrazol-3-yl)-3,6-dihydro-2H-pyridin-1-yl]-methanone (Compound 586)

Prepared using similar procedure as in Example 475 (compound 575). ¹H NMR (d₆-DMSO, 300 MHz) δ 8.83 (s, 1H), 8.56 (s, 1H), 8.21 (s, 1H), 7.84 (t, J=1.80 Hz, 1H), 7.39 (d, J=1.50 Hz, 1H, 7.33 (d, J=1.50 Hz, 1H), 6.29 (d, 1.80 Hz, 1H), 6.10 (s, 0.5H), 5.96 (s, 0.5H), 4.38 (d, J=18.30 Hz, 2H), 3.86 (m, 5H), 2.51 (m, 2H). MS (ESI) m/z=476.0 (MH⁺).

### Example 487

### (3-Chloro-6-furan-3-yl-8-trifluoromethyl-imidazo[1,2-a]pyridine-2-yl)-(2′-fluoro-3,6-dihydro-2H-[4,4′]bipyridinyl-1-yl)-methanone (Compound 587)

Prepared using similar procedure as in Example 475 (compound 575). ¹H NMR (d₆-DMSO, 300 MHz) δ 8.83 (s, 1H), 8.56 (s, 1H), 8.22 (m, 2H), 7.84 (m, 1H), 7.46 (d, J=5.40 Hz, 1H), 7.33 (s, 1H), 7.24 (s, 1H), 6.71 (s, 0.5H), 6.58 (s, 0.5H), 4.44 (d, J=27.90 Hz, 2H), 3.90 (m, 2H), 2.62 (m, 2H). MS (ESI) m/z=491.0 (MH⁺).

### Example 488

### (3-Chloro-6-furan-3-yl-8-trifluoromethyl-imidazo[1,2-a]pyridin-2-yl)-(5-thiazol-4-yl-3,4-dihydro-2H-pyridin-1-yl)-methanone (Compound 588)

### Step 1: 5-Trifluoromethanesulfonyloxy-3,4-dihydro-2H-pyridine-1-carboxylic Acid Tert-butyl Ester and 5-trifluoromethanesulfonyloxy-3,6-dihydro-2H-pyridine-1-carboxylic Acid Tert-butyl Ester

Using similar procedure as in Example 474, Step 1,1-Boc-3-piperidone was treated with LDA and N-phenyl bis(trifluoromethanesulfonimide) to give a mixture of triflates (2:3 ratio).

Data for 5-trifluoromethanesulfonyloxy-3,4-dihydro-2H-pyridine-1-carboxylic acid tert-butyl ester (JM-2549-82A): ¹H NMR (CDCl₃, 300 MHz) δ 7.14 (s, 1H), 3.45 (t, J=5.70 Hz, 2H), 2.41 (t, J=6.30 Hz, 2H), 1.86 (m, 2H), 1.43 (s, 9H); MS (ESI) m/z=276 (MH⁺-^{t}Bu)

Data for 5-trifluoromethanesulfonyloxy-3,6-dihydro-2H-pyridine-1-carboxylic acid tert-butyl ester: ¹H NMR (d₆-DMSO, 300 MHz) δ 6.10 (m, 1H), 4.03 (s, 2H), 3.45 (t, J=5.40 Hz, 2H), 2.25 (m, 2H), 1.43 (s, 9H); MS (ESI) m/z=276 (MH⁺-^{t}Bu)

### Step 2: 5-(4,4,5,5-Tetramethyl-[1,3,2]dioxaborolan-2-yl)-3,4-dihydro-2H-pyridine-1-carboxylic Acid Tert-butyl Ester

5-Trifluoromethanesulfonyloxy-3,4-dihydro-2H-pyridine-1-carboxylic acid tert-butyl ester (700 mg, 2.11 mmol) was dissolved in 1,4-dioxane (15 mL) and added under N₂ (g) to a degassed mixture of potassium acetate (622 mg, 6.34 mmol), Pd(dppf)Cl₂*CH₂Cl₂ (52 mg, 0.06 mmol), dppf (35 mg, 0.06 mmol), bis-pinacolato diborane (590 mg, 2.32 mmol) and the reaction mixture heated at 80° C. overnight. The mixture was concentrated on silica and subjected to flash column chromatography (15-50% EtOAc/n-hexane gradient) to afford 5-(4,4,5,5-tetramethyl-[1,3,2]dioxaborolan-2-yl)-3,4-dihydro-2H-pyridine-1-carboxylic acid tert-butyl ester (320 mg, 49%) as white semi-solid. MS (ESI) m/z=254.1 (MH⁺-^{t}Bu).

### Step 3: 5-Thiazol-4-yl-3,4-dihydro-2H-pyridine-1-carboxylic Acid tert-butyl Ester

Prepared similar procedure as in Example 456, Step 1.

¹H NMR (CDCl₃, 300 MHz) δ 8.75 (s, 1H), 7.89 (s, 1H), 6.89 (s, 1H), 3.63 (m, 2H), 2.44 (t, J=6.60 Hz, 2H), 1.97 (m, 2H), 1.53 (s, 9H); MS (ESI) m/z=211.1 (MH⁺-^{t}Bu)

### Step 4: 5-Thiazol-4-yl-1,2,3,4-tetrahydro-pyridine Hydrochloride

5-Thiazol-4-yl-3,4-dihydro-2H-pyridine-1-carboxylic acid tert-butyl ester underwent HCl deprotection to give 5-thiazol-4-yl-1,2,3,4-tetrahydro-pyridine hydrochloride. MS (ESI) m/z=167.1 (MH⁺).

### Step 5: (3-Chloro-6-furan-3-yl-8-trifluoromethyl-imidazo[1,2-a]pyridin-2-yl)-(5-thiazol-4-yl-3,4-dihydro-2H-pyridin-1-yl)-methanone (Compound 588)

Prepared using standard HATU coupling of the above amine. ¹H NMR (CDCl₃, 300 MHz) δ 9.31 (s, 1H), 8.81 (s, 1H), 8.37 (s, 1H), 7.85 (s, 1H), 7.80 (s, 1H), 7.58 (m, 1H), 7.25 (m, 1H), 6.74 (m, 1H), 3.96 (m, 2H), 2.66 (m, 2H), 2.18 (m, 2H). MS (ESI) m/z=479.0 (MH⁺).

### Example 489

### (3-Chloro-6-furan-3-yl-8-trifluoromethyl-imidazo[1,2-a]pyridin-2-yl)-(5-thiazol-4-yl-3,6-dihydro-2H-pyridin-1-yl)-methanone (Compound 589)

### Step 1: 5-(4,4,5,5-Tetramethyl-[1,3,2]dioxaborolan-2-yl)-3,6-dihydro-2H-pyridine-1-carboxylic Acid Tert-butyl Ester

Prepared using similar procedure as in Example 488, Step 2. MS (ESI) m/z=254.1 (MH⁺-^{t}Bu).

### Step 2: 5-Thiazol-4-yl-3,6-dihydro-2H-pyridine-1-carboxylic Acid tert-butyl Ester

Prepared using similar procedure as in Example 488, Step 3. ¹H NMR (CDCl₃, 300 MHz) δ 8.75 (s, 1H), 7.06 (s, 1H), 6.81 (s, 1H), 4.28 (m, 2H), 3.54 (t, J=5.40 Hz, 2H), 2.33 (m, 2H), 1.47 (s, 9H); MS (ESI) m/z=211.1 (MH⁺-^{t}Bu)

### Step 3: 5-Thiazol-4-yl-1,2,3,6-tetrahydro-pyridine Hydrochloride

Prepared using similar procedure as in Example 488, Step 4. ¹H NMR (d₆-DMSO, 300 MHz) δ 9.30 (s, 1H), 7.76 (s, 1H), 6.78 (m, 1H), 4.70 (m, 3H), 3.99 (m, 2H), 3.25 (m, 2H). MS (ESI) m/z=167.1 (MH⁺).

### Step 4: (3-Chloro-6-furan-3-yl-8-trifluoromethyl-imidazo[1,2-a]pyridin-2-yl)-(5-thiazol-4-yl-3,6-dihydro-2H-pyridin-1-yl)-methanone (Compound 589)

Prepared using standard HATU coupling of the above amine. ¹H NMR (d₆-DMSO, 300 MHz) δ 9.14 (d, J=1.80 Hz, 1H), 8.83 (s, 1H), 8.56 (s, 1H), 8.21 (s, 1H), 7.84 (d, J=1.50 Hz, 1H), 7.76 (s, 1H), 7.33 (s, 1H), 6.79 (m, 1H), 4.63 (d, J=24.60 Hz, 2H), 3.83 (m, 2H), 2.42 (m, 2H). MS (ESI) m/z=479.0 (MH⁺).

### Example 490

### (3-Chloro-6-furan-3-yl-8-trifluoromethyl-imidazo[1,2-a]pyridine-2-yl)-[5-(2-fluoro-phenyl)-3,4-dihydro-2H-pyridin-1-yl]-methanone (Compound 590)

### Step 1: 5-(2-Fluoro-phenyl)-3,4-dihydro-2H-pyridine-1-carboxylic Acid Tert-butyl Ester

5-Trifluoromethanesulfonyloxy-3,4-dihydro-2H-pyridine-1-carboxylic acid tert-butyl ester underwent Suzuki reaction with 2-fluorophenylboronic acid using conditions as in Example 455, Step 1. ¹H NMR (CDCl₃, 300 MHz) δ 7.28 (m, 1H), 7.17 (m, 4H), 3.53 (m, 2H), 2.35 (t, J=6.30 Hz, 2H), 1.86 (m, 2H), 1.43 (s, 9H); MS (ESI) m/z=222.1 (MH⁺-^{t}Bu)

### Step 2: 5-(2-Fluoro-phenyl)-1,2,3,4-tetrahydro-pyridine Hydrochloride

5-(2-Fluoro-phenyl)-3,4-dihydro-2H-pyridine-1-carboxylic acid tert-butyl ester underwent HCl deprotection to give 5-(2-fluoro-phenyl)-1,2,3,4-tetrahydro-pyridine hydrochloride. MS (ESI) m/z=178.0 (MH⁺).

### Step 3: (3-Chloro-6-furan-3-yl-8-trifluoromethyl-imidazo[1,2-a]pyridine-2-yl)-[5-(2-fluoro-phenyl)-3,4-dihydro-2H-pyridin-1-yl]-methanone (Compound 590)

Prepared using standard HATU coupling of the above amine. ¹H NMR (CDCl₃, 300 MHz) δ 8.33 (s, 1H), 7.90 (s, 1H), 7.85 (s, 1H), 7.79 (s, 1H), 7.59 (t, J=1.50 Hz, 1H), 7.38 (m, 1H), 7.08 (m, 3H), 6.74 (d, J=1.80 Hz, 1H), 4.11 (m, 0.5H), 3.97 (m, 1.5H), 2.59 (t, J=6.30 Hz, 2H), 2.10 (m, 2H). MS (ESI) m/z=490.0 (MH⁺).

### Example 491

### (3-Chloro-6-furan-3-yl-8-trifluoromethyl-imidazo[1,2-a]pyridine-2-yl)-[5-(2-fluoro-phenyl)-3,6-dihydro-2H-pyridin-1-yl]-methanone (Compound 591)

### Step 1: 5-(2-Fluoro-phenyl)-3,6-dihydro-2H-pyridine-1-carboxylic Acid Tert-butyl Ester

5-Trifluoromethanesulfonyloxy-3,6-dihydro-2H-pyridine-1-carboxylic acid tert-butyl ester underwent Suzuki reaction with 2-fluorophenylboronic acid using conditions as in Example 455, Step 1. ¹H NMR (CDCl₃, 300 MHz) δ 7.25 (m, 2H), 7.16 (m, 3H), 3.61 (m, 2H), 2.44 (t, J=6.30 Hz, 2H), 1.96 (m, 2H), 1.48 (s, 9H); MS (ESI) m/z=222.1 (MH⁺-^{t}Bu)

### Step 2: 5-(2-Fluoro-phenyl)-1,2,3,6-tetrahydro-pyridine Hydrochloride

5-(2-Fluoro-phenyl)-3,6-dihydro-2H-pyridine-1-carboxylic acid tert-butyl ester underwent HCl deprotection to give 5-(2-fluoro-phenyl)-1,2,3,6-tetrahydro-pyridine hydrochloride. MS (ESI) m/z=178.0 (MH⁺).

### Step 3: (3-Chloro-6-furan-3-yl-8-trifluoromethyl-imidazo[1,2-a]pyridine-2-yl)-[5-(2-fluoro-phenyl)-3,6-dihydro-2H-pyridin-1-yl]-methanone (Compound 591)

Prepared using standard HATU coupling of the above amine. ¹H NMR (d₆-DMSO, 300 MHz) δ 8.80 (d, J=12.30 Hz, 1H), 8.53 (d, J=7.50 Hz, 1H), 8.18 (d, J=10.80 Hz, 1H), 7.82 (m, 1H), 7.39 (m, 1H), 7.28 (m, 3H), 7.18 (m, 1H), 6.14 (m, 1H), 4.62 (s, 1H), 4.47 (s, 1H), 3.82 (m, 2H). 2.39 (m, 2H). MS (ESI) m/z=490.1 (MH⁺).

### Example 492

### [3-Chloro-6-(1H-pyrazol-4-yl)-8-trifluoromethyl-imidazo[1,2-a]pyridin-2-yl)-2,5-dihydro-pyrrol-1-yl]-methanone (Compound 592)

### Step 1: 5-(4,4,5,5-Tetramethyl-[1,3,2]dioxaborolan-2-yl)-2,5-dihydro-pyrrole-1-carboxylic Acid Tert-butyl Ester

3-Trifluoromethanesulfonyloxy-2,5-dihydro-pyrrole-1-carboxylic acid tert-butyl ester was converted 5-(4,4,5,5-tetramethyl-[1,3,2]dioxaborolan-2-yl)-2,5-dihydro-pyrrole-1-carboxylic acid tert-butyl ester using similar procedure as in Example 423, Step 2. ¹H NMR (CDCl₃, 300 MHz) δ 6.42 (m, 1H), 4.20 (s, 2H), 4.15 (d, J=3.00 Hz, 2H), 1.46 (s, 9H), 1.27 (s, 12H); MS (ESI) m/z=240.1 (MH⁺-^{t}Bu)

### Step 2: 3-(3-Fluoro-pyridin-2-yl)-2,5-dihydro-pyrrole-1-carboxylic Acid Tert-butyl Ester

5-(4,4,5,5-Tetramethyl-[1,3,2]dioxaborolan-2-yl)-2,5-dihydro-pyrrole-1-carboxylic acid tert-butyl ester underwent Suzuki reaction with 2-bromo-3-fluoropyridine using conditions as in Example 455, Step 1. ¹H NMR (CDCl₃, 300 MHz) δ 8.40 (m, 1H), 7.42 (m, 1H), 7.22 (m, 1H), 6.68 (m, 1H), 6.62 (m, 2H), 4.43 (m, 2H), 1.50 (s, 9H); MS (ESI) m/z=210 (MH⁺-^{t}Bu)

### Step 3: 2-(2,5-Dihydro-1H-pyrrol-3-yl)-3-fluoro-pyridine Hydrochloride

3-(3-Fluoro-pyridin-2-yl)-2,5-dihydro-pyrrole-1-carboxylic acid tert-butyl ester underwent HCl deprotection to give 2-(2,5-Dihydro-1H-pyrrol-3-yl)-3-fluoro-pyridine hydrochloride. ¹H NMR (d₆-DMSO, 300 MHz) δ 9.72 (s, 1H), 8.46 (m, 1H), 7.85 (m, 1H), 7.50 (m, 1H), 6.65 (m, 1H), 4.42 (m, 2H), 4.24 (m, 2H). MS (ESI) m/z=165.1 (MH⁺).

### Step 4: [3-Chloro-6-(1H-pyrazol-4-yl)-8-trifluoromethyl-imidazo[1,2-a]pyridin-2-yl)-2,5-dihydro-pyrrol-1-yl]-methanone (Compound 592)

Prepared using standard HATU coupling of the above amine. ¹H NMR (d₆-DMSO, 300 MHz) δ 8.86 (s, 1H), 8.49 (m, 0.5H), 8.42 (m, 2.5H), 8.23 (s, 1H), 7.83 (t, J=11.70 Hz, 1H), 7.45 (m, 1H), 6.78 (s, 1H), 5.14 (m, 1H), 4.98 (m, 1H), 4.84 (m, 1H), 4.64 (m, 1H). MS (ESI) m/z=477.0 (MH⁺).

### Example 493

### (3-Chloro-6-furan-3-yl-8-trifluoromethyl-imidazo[1,2-a]pyridin-2-yl)-(3-phenoxy-pyrrolidin-1-yl)-methanone (Compound 593)

A mixture of 3-chloro-6-furan-2-yl-8-trifluoromethyl-imidazo[1,2-a]pyridine-2-carboxylic acid (40 mg, 0.14 mmol), amine (0.14 mmol), HATU (54 mg, 0.14 mmol), and N,N-diisopropylethylamine (0.08 mL, 0.42 mmol in DMF (0.8 mL) was stirred at room temperature. After 1.5 hours, the mixture was diluted with EtOAc (20 mL) and washed with saturated aqueous NaHCO₃ (10 mL), then brine (10 mL). The filtrate was dried (Na₂SO₄), filtered and concentrated. Column chromatography [n-hex/EtOAc (5:4 v/v)] of the crude material gave compound 593 (51 mg, 74%) as a white powder. ¹H NMR (d₆-DMSO, 300 MHz) [symbol] 2.23 (m, 2H), 3.38 (m, 1H), 3.81 (m, 2H), 4.09 (m, 1H), 5.12 (m, 1H), 4.09 (m, 1H), 6.95 (m, 3H), 7.28 (m, 3H), 7.83 (m, 1H), 8.18 (dd, 1H, J=6.6 Hz), 8.55 (d, 1H, J=3.6 Hz), 8.81 (d, 1H, J=10.2 Hz), MS (ESI) m/z=477 (MH⁺).

### Example 494

### (3-Chloro-6-furan-3-yl-8-trifluoromethyl-imidazo[1,2-a]pyridin-2-yl)-(4-phenyl-3,6-dihydro-2H-pyridin-1-yl)-methanone (Compound 594)

Prepared using standard HATU coupling. ¹H NMR (d₆-DMSO, 300 MHz) δ 2.53 (bs, 2H), 3.80 (m, 2H), 4.28 (bd, 2H), 6.19 (bd, 1H), 7.25 (m, 4H), 7.39 (m, 2H), 7.76 (s, 1H), 8.13 (s, 1H), 8.48 (s, 1H), 8.75 (s, 1H); MS (ESI) m/z=472 (MH⁺).

### Example 495

### (3-Chloro-6-furan-3-yl-8-trifluoromethyl-imidazo[1,2-a]pyridin-2-yl)-[4-(4-fluoro-phenyl)-3,6-dihydro-2H-pyridin-1-yl]-methanone (Compound 595)

Prepared using standard HATU coupling. ¹H NMR (d₆-DMSO, 300 MHz) δ 2.52 (bd, 2H), 3.82 (m, 2H), 4.28 (bd, 2H), 6.03 & 6.16 (bd, 1H), 7.08 (m, 2H), 7.24 (s, 1H), 7.44 (m, 2H) 7.57 (s, 1H), 8.12 (s, 1H), 8.48 (s, 1H), 8.74 (s, 1H); MS (ESI) m/z=491 (MH⁺).

### Example 496

### (3-Chloro-6-furan-3-yl-8-trifluoromethyl-imidazo[1,2-a]pyridin-2-yl)-[4-(2,4-difluoro-phenyl)-piperazin-1-yl]-methanone (Compound 596)

Prepared using standard HATU coupling. ¹H NMR (d₆-DMSO, 300 MHz) δ 3.04 (bd, 4H), 3.83 (bs, 4H), 7.00 (m, 1H), 7.10 (m, 1H), 7.20 (m, 1H) 7.31 (m, 1H), 7.83 (t, 1H, J=1.5 Hz), 8.19 (s, 1H), 8.55 (s, 1H), 8.81 (s, 1H); MS (ESI) m/z=512 (MH⁺).

### Example 497

### (3-Chloro-6-furan-3-yl-8-trifluoromethyl-imidazo[1,2-a]pyridin-2-yl)-(4-pyrimidin-2-yl-piperazin-1-yl)-methanone (Compound 597)

Prepared using standard HATU coupling. ¹H NMR (d₆-DMSO, 300 MHz) δ 2.50 (m, 4H), 3.77 (m, 4H), 6.66 (t, 1H, J=4.5 Hz), 7.32 (m, 1H), 7.83 (m, 1H), 8.20 (s, 1H), 8.37 (s, 1H), 8.39 (s, 1H), 8.55 (s, 1H), 8.82 (s, 1H); MS (ESI) m/z=478 (MH⁺).

### Example 498

### [3-chloro-6-(furan-3-yl)-8-(trifluoromethyl)imidazo[1,2-a]pyridin-2-yl][4-(thiophen-2-yl)piperidin-1-yl]methanone (Compound 598)

Prepared using standard HATU coupling. ¹H NMR (d₆-DMSO, 300 MHz) δ 1.55 (m, 2H), 1.98 (m, 2H), 2.94 (m, 1H), 3.24 (m, 2H), 4.15 (m, 1H), 4.61 (m, 1H), 6.95 (m, 2H), 7.32 (s, 2H), 7.82 (m, 1H), 8.18 (s, 1H), 8.55 (s, 1H), 8.81 (s, 1H), MS (ESI) m/z=479 (MH⁺).

### Example 499

### (3-Chloro-6-furan-3-yl-8-trifluoromethyl-imidazo[1,2-a]pyridin-2-yl)-(3-phenylamino-pyrrolidin-1-yl)-methanone (Compound 599)

Prepared using standard HATU coupling. ¹H NMR (d₆-DMSO, 300 MHz) δ 1.86 (m, 1H), 2.16 (m, 1H), 3.42-4.50 (m, 5H), 5.84 (dd, 1H, J=6.3 & 9.0 Hz), 6.47 (m, 1H), 6.57 (m, 1H), 6.57 (d, 1H, J=7.5 Hz), 6.98 (dd, 1H, J=8.1, 7.2 Hz), 7.03 (dd, 1H, J=8.4 & 7.2 Hz), 7.26 (m, 1H), 7.77 (m, 1H), 8.13 (d, 1H, J=4.5 Hz), 8.49 (d, 1H, J=4.2 Hz), 8.75 (d, 1H, J=7.5 Hz); MS (ESI) m/z=476 (MH⁺).

### Example 500

### N-[1-(3-Chloro-6-furan-3-yl-8-trifluoromethyl-imidazo[1,2-a]pyridine-2-carbonyl)-pyrrolidin-3-yl]-N-phenyl-acetamide (Compound 600)

To a solution of (3-chloro-6-furan-3-yl-8-trifluoromethyl-imidazo[1,2-a]pyridin-2-yl)-(3-phenylamino-pyrrolidin-1-yl)-methanone (0.06 mmol) in THF (2 mL) was added Et₃N (1.2 mmol). After 15 min, acetyl chloride (0.025 mL, 0.18 mmol) was added and the solution was stirred at 60° C. for 3 hours. The solvent was evaporated and the mixture was carefully poured into ice-water (2 mL) to give a white precipitate which was filtered and dried under high vacuum to give N-[1-(3-chloro-6-furan-3-yl-8-trifluoromethyl-imidazo[1,2-a]pyridine-2-carbonyl)-pyrrolidin-3-yl]-N-phenyl-acetamide (90%) as a light yellow solid. ¹H NMR (d₆-DMSO, 300 MHz) δ 1.76 (m, 1H), 2.13 (m, 1H), 3.14 (m, 1H), 3.34 (s, 3H), 3.54 (m, 1H), 3.80 (m, 1H), 3.83 (m, 0.5H), 3.99 (m, 0.5H), 5.09 (m, 1H), 7.30 (3, 3H), 7.43 (m, 3H), 7.82 (m, 1H), 8.16 (bd, 1H, J=8.4 Hz), 8.54 (bd, 1H, J=5.7 Hz), 8.79 (dd, 1H, J=6.6 Hz); MS (ESI) m/z=518 (MH⁺).

### Example 501

### 1-(3-Chloro-6-furan-3-yl-8-trifluoromethyl-imidazo[1,2-a]pyridine-2-carbonyl)-4-phenyl-piperidine-4-carbonitrile (Compound 601)

Prepared using standard HATU coupling. ¹H NMR (d₆-DMSO, 300 MHz) δ 2.14 (m, 2H), 2.27 (m, 2H), 3.12 (m, 1H), 3.42 (m, 1H), 4.42 (d, 1H, J=3.8 Hz), 4.74 (d, 1H, J=12.9 Hz), 7.37 (m, 2H), 7.42 (m, 2H), 7.57 (m, 2H) 7.82 (t, 1H, J=1.2 Hz), 8.20 (s, 1H), 8.55 (s, 1H), 8.81 (s, 1H); MS (ESI) m/z=500 (MH⁺).

### Example 502

### 3-Chloro-6-furan-3-yl-8-trifluoromethyl-imidazo[1,2-a]pyridine-2-carboxylic Acid (phenyl-thiophen-2-yl-methyl)-amide (Compound 602)

Prepared using standard HATU coupling. ¹H NMR (d₆-DMSO, 300 MHz) δ 3.30 (bs, 1H), 6.53 (m, 1H), 6.95 (bd, 2H), 7.36 (m, 5H), 7.81 (bs, 1H), 8.21 (bs, 1H), 8.54 (bs, 1H), 8.79 (bs, 1H), 8.93 (m, 1H); MS (ESI) m/z=503 (MH⁺).

### Example 503

### 2-[3-Chloro-6-(furan-3-yl)-8-(trifluoromethyl)imidazo[1,2-a]pyridin-2-yl]-N-(thiophen-2-ylmethyl)acetamide (Compound 603)

### Step 1: (6-Bromo-3-chloro-8-trifluoromethyl-imidazo[1,2-a]pyridin-2-yl)-acetic Acid

A mixture of (6-bromo-8-trifluoromethyl-imidazo[1,2-a]pyridin-2-yl)-acetic acid (0.1 g, 0.3 mmol) and N-chlorosuccinimide (50 mg, 0.36 mmol) was stirred at 60° C. in DMF (1 mL) for 12 hours. The mixture was diluted with EtOAc (10 mL) and washed with water (10 mL), 1M sodium thiosulfate solution (10 mL), and brine (10 mL). The filtrate was dried (Na₂SO₄), filtered and concentrated to give (6-bromo-3-chloro-8-trifluoromethyl-imidazo[1,2-a]pyridin-2-yl)-acetic acid (80%) as a brown solid. MS (ESI) m/z=358 (MH⁺).

### Step 2: 2-(6-Bromo-3-chloro-8-trifluoromethyl-imidazo[1,2-a]pyridin-2-yl)-N-thiophen-2-ylmethyl-acetamide

Prepared using standard HATU coupling of (6-bromo-3-chloro-8-trifluoromethyl-imidazo[1,2-a]pyridin-2-yl)-acetic acid. MS (ESI) m/z=454 (MH⁺).

### Step 3. 2-[3-Chloro-6-(furan-3-yl)-8-(trifluoromethyl)imidazo[1,2-a]pyridin-2-yl]-N-(thiophen-2-ylmethyl)acetamide (compound 603)

Prepared using Suzuki coupling of 2-(6-bromo-3-chloro-8-trifluoromethyl-imidazo[1,2-a]pyridin-2-yl)-N-thiophen-2-ylmethyl-acetamide.

¹H NMR (d₆-DMSO, 300 MHz) δ 3.69 (s, 2H), 4.44 (d, 1H, J=6.0 Hz), 6.94 (m, 1H), 7.00 (m, 1H), 7.30 (m, 1H), 7.39 (m, 1H), 7.84 (m, 1H), 8.09 (s, 1H), 8.52 (s, 1H), 8.67 (s, 1H), 8.76 (s, 1H); MS (ESI) m/z=441 (MH⁺).

### Example 504

### 2-[3-chloro-6-(furan-3-yl)-8-(trifluoromethyl)imidazo [1,2-a]pyridin-2-yl]-1-[3-(3-fluorophenyl)pyrrolidin-1-yl]ethanone (Compound 604)

Prepared using similar procedure as in Example 503

¹H NMR (d₆-DMSO, 300 MHz) δ 2.01 (m, 1H), 2.18 (m, 1H), 3.31-3.63 (m, 4H), 3.80 (m, 2.5H), 4.10 (m, 0.5H), 7.01 (m, 1H), 7.12 (m, 2H), 7.23 (s, 1H), 7.30 (m, 1H), 7.76 (s, 1H), 8.80 (s, 1H), 8.45 (s, 1H), 8.69 (s, 1H); MS (ESI) m/z=493 (MH⁺).

### Example 505

### (4-Benzoimidazol-1-yl-piperidin-1-yl)-(3-chloro-6-furan-3-yl-8-trifluoromethyl-imidazo[1,2-a]pyridin-2-yl)-methanone (Compound 605)

Prepared using standard HATU coupling. MS (ESI) m/z=515 (MH⁺).

### Example 506

### [3-Chloro-6-(1H-pyrazol-4-yl)-8-trifluoromethyl-imidaz [1,2-a]pyridin-2-yl]-[4-(2-fluoro-phenyl)-piperidin-1-yl]-methanone (Compound 606)

Prepared using standard HATU coupling. ¹H NMR (d₆-DMSO, 300 MHz) δ 1.66 (m, 2H), 1.85 (m, 2H), 2.84 (m, 1H), 3.16 (m, 2H), 4.14 (d, 1H, J=13.8 Hz), 4.63 (d, 1H, J=12.9 Hz), 5.46 (bs, 1H) 7.11 (m, 2H), 7.19 (m, 1H), 7.25 (m, 1H), 8.12 (s, 1H), 8.32 (bs, 2H), 8.74 (s, 1H); MS (ESI) m/z=492 (MH⁺).

### Example 507

### 2-{1-[3-Chloro-6-(1H-pyrazol-4-yl)-8-trifluoromethyl-imidazo[1,2-a]pyridine-2-carbonyl]-piperidin-4-yl}-benzonitrile (Compound 607)

Prepared using standard HATU coupling. ¹H NMR (d₆-DMSO, 300 MHz) δ 1.69-1.95 (m, 2H), 2.95-3.26 (m, 4H), 4.25-4.71 (bm, 3H), 7.41 (m, 2H), 7.55 (d, 1H, J=7.8 Hz), 7.66 (m, 1H), 7.80 (d, 1H, J=7.8 Hz), 8.17 (s, 1H), 8.38 (bs, 2H), 8.81 (s, 1H); MS (ESI) m/z=499 (MH⁺).

### Example 508

### [3-Bromo-6-(1H-pyrazol-4-yl)-8-trifluoromethyl-imidazo[1,2-a]pyridin-2-yl]-[4-(4-fluoro-phenyl)-3,6-dihydro-2H-pyridin-1-yl]-methanone (Compound 608)

Prepared using standard HATU coupling. ¹H NMR (d₆-DMSO, 300 MHz) δ 2.52 (m, 2H), 3.73 & 3.84 (t, 2H, J=6.0 Hz), 4.26 (bd, 2H), 6.15 (m, 1H), 7.14 (m, 2H), 7.44 (m, 2H), 7.61 (m, 1H), 8.15 (s, 1H), 8.36 (s, 1H), 8.71 (s, 1H); MS (ESI) m/z=535 (MH⁺).

### Example 509

### [3-Bromo-6-(1H-pyrazol-4-yl)-8-trifluoromethyl-imidazo[1,2-a]pyridin-2-yl]-(4-thiazol-4-yl-3,6-dihydro-2H-pyridin-1-yl)-methanone (Compound 609)

Prepared using standard HATU coupling. ¹H NMR (d₆-DMSO, 300 MHz) δ 2.59 (m, 2H), 3.77 (m, 4H), 4.31 (m, 2H), 6.52 & 6.70 (bd, 1H), 7.61 (m, 1H), 8.19 (s, 1H), 8.41 (s, 2H), 8.76 (s, 1H), 9.09 (m, 1H); MS (ESI) m/z=524 (MH⁺).

### Example 510

### [3-Bromo-6-(1H-pyrazol-4-yl)-8-trifluoromethyl-imidazo[1,2-a]pyridin-2-yl]-(4-thiazol-2-yl-piperazin-1-yl)-methanone (Compound 610)

Prepared using standard HATU coupling. ¹H NMR (d₆-DMSO, 300 MHz) δ 3.74 (m, 4H), 3.89 (m, 4H), 7.10 (d, 1H, J=4.2 Hz), 7.44 (d, 1H, J=4.2 Hz), 8.23 (m, 1H), 8.43 (s, 2H), 8.78 (s, 1H), 9.09 (m, 1H); MS (ESI) m/z=524 (MH⁺).

### Example 511

### [3-Bromo-6-(1H-pyrazol-4-yl)-8-trifluoromethyl-imidazo[1,2-a]pyridin-2-yl]-(4-thiazol-2-yl-piperidin-1-yl)-methanone (Compound 611)

Prepared using standard HATU coupling. ¹H NMR (d₆-DMSO, 300 MHz) δ 1.68 (m, 2H), 1.99 (m, 2H), 2.16 (m, 0.5H) 3.02 (m, 1.5H), 3.22-3.41 (m, 2.5H), 4.54 (m, 0.5H), 7.62 (d, 1H, J=3.3 Hz), 7.73 (d, 1H, J=3.6 Hz), 8.17 (s, 1H), 8.37 (s, 2H), 8.73 (s, 1H); MS (ESI) m/z=524 (MH⁺).

### Example 512

### [6-(1H-pyrazol-4-yl)-8-(trifluoromethyl)imidazo[1,2-a]pyridin-2-yl][4-(1,3-thiazol-2-yl)-3,6-dihydropyridin-1(2H)-yl]methanone (Compound 612)

Prepared using standard HATU coupling. ¹H NMR (d₆-DMSO, 300 MHz) δ 2.72 (m, 2H), 4.33 (bm, 2H), 4.89 (m, 2H), 6.62 and 6.69 (bs, 1H), 7.65 (d, 1H, J=3.0 Hz), 7.80 (d, 1H, J=3.3 Hz), 8.09 (s, 1H), 8.22 (s, 2H), 8.38 (s, 1H), 9.41 (s, 1H); MS (ESI) m/z=446 (MH⁺).

### General Procedure for the Examples 513-515

A mixture of (3-chloro-6-furan-3-yl-8-trifluoromethyl-imidazo[1,2-a]pyridin-2-yl)-[4-(4,4,5,5-tetramethyl-[1,3,2]dioxaborolan-2-yl)-3,6-dihydro-2H-pyridin-1-yl]-methanone (52 mg, 0.10 mmol), R—Br (0.25 mmol) and Pd(dppf)Cl₂*CH₂Cl₂ (4 mg, 0.005 mmol) in 2M Na₂CO₃ (0.5 mL) and 1,4-dioxane (1.2 mL) was heated at 100° C. for 12 hours. The mixture was diluted with EtOAc (25 mL) and washed with saturated aqueous NaHCO₃ (10 mL), and brine (10 mL). The extracts were dried (Na₂SO₄), filtered and concentrated. Preparative HPLC purification (30-100% ACN gradient) of the crude product gave the final product (˜25% Yield) as a white powder.

### Example 513

### (3-Chloro-6-furan-3-yl-8-trifluoromethyl-imidazo[1,2-a]pyridin-2-yl)-(4-thiophen-2-yl-3,6-dihydro-2H-pyridin-1-yl)-methanone (Compound 613)

¹H NMR (d₆-DMSO, 300 MHz) δ 2.54 (bs, 2H), 3.80 (m, 2H), 4.26 (m, 2H), 5.98 & 6.14 (bs, 1H), 6.97 (m, 1H), 7.07 (m, 1H), 7.27 (m, 1H), 7.36 (t, 1H, J=4.5), 7.78 (m, 1H), 8.16 (m, 1H), 8.50 (s, 1H), 8.77 (s, 1H), MS (ESI) m/z=479 (MH⁺).

### Example 514

### 2-[1-(3-Chloro-6-furan-3-yl-8-trifluoromethyl-imidazo[1,2-a]pyridine-2-carbonyl)-1,2,3,6-tetrahydro-pyridin-4-yl]-6-fluoro-benzonitrile (Compound 614)

Prepared using similar procedure as in Example 413 (compound 513). ¹H NMR (d₆-DMSO, 300 MHz) δ 2.56 (m 2H), 3.83 (m, 2H), 4.33 (m, 2H), 6.03 & 6.16 (bd, 1H), 7.27 (m, 1H), 7.40 (m, 2H) 7.70 (m, 1H), 7.77 (m, 1H), 8.15 (s, 1H), 8.50 (s, 1H), 8.78 (s, 1H); MS (ESI) m/z=516 (MH⁺).

### Example 515

### (3-Chloro-6-furan-3-yl-8-trifluoromethyl-imidazo[1,2-a]pyridin-2-yl)-[4-(2-methyl-thiazol-4-yl)-3,6-dihydro-2H-pyridin-1-yl]-methanone (Compound 615)

Prepared using similar procedure as in Example 413 (compound 513). ¹H NMR (d₆-DMSO, 300 MHz) δ 2.54 (bs, 2H), 2.64 (s, 3H), 3.86 (m, 2H), 4.32 (m, 2H), 6.63 & 6.47 (bd, 1H), 7.31 (s, 1H), 7.38 (m, 1H), 7.82 (s, 1H), 8.19 (s, 1H), 8.55 (s, 1H), 8.81 (s, 1H), MS (ESI) m/z=479 (MH⁺).

### Example 516

### (3-Chloro-6-furan-3-yl-8-trifluoromethyl-imidazo[1,2-a]pyridin-2-yl)-[4-(2,6-difluoro-3-methoxy-phenyl)-3,6-dihydro-2H-pyridin-1-yl]-methanone (Compound 616)

A mixture of trifluoro-methanesulfonic acid 1-(3-chloro-6-furan-3-yl-8-trifluoromethyl-imidazo[1,2-a]pyridine-2-carbonyl)-1,2,3,6-tetrahydro-pyridin-4-yl ester (compound 574, 50 mg, 0.0.10 mmol), 2,6-difluoro-3-methoxyphenylboronic acid (0.25 mmol) and Pd(dppf)Cl₂.CH₂Cl₂ (4 mg, 0.005 mmol) in 2M Na₂CO₃ (0.5 mL) and ACN (1.2 mL) was heated at 100° C. for 12 hours. The mixture was diluted with EtOAc (25 mL) and washed with saturated aqueous NaHCO₃ (10 mL), and brine (10 mL). The extracts were dried (Na₂SO₄), filtered and concentrated. Preparative HPLC purification (30-100% ACN gradient) of the crude product gave the final product (45% yield) as a white powder. ¹H NMR (d₆-DMSO, 300 MHz) δ 2.44 (m, 2H), 3.87 (m, 2H), 4.30 (m, 2H), 5.95 & 5.81 (bd, 1H), 7.06 (m, 2H), 7.31 (m, 1H), 7.82 (t, 1H, J=1.8 Hz), 8.18 (s, 1H), 8.54 (s, 1H), 8.81 (s, 1H); MS (ESI) m/z=539 (MH⁺).

### Example 517

### (3-Chloro-6-furan-3-yl-8-trifluoromethyl-imidazo[1,2-a]pyridin-2-yl)-(2,6-difluoro-3′,6′-dihydro-2′H-[3,4′]bipyridinyl-1′-yl)-methanone (Compound 617)

Prepared using similar procedure as in Example 516 (compound 616). ¹H NMR (d₆-DMSO, 300 MHz) δ 2.54 (m, 2H), 3.85 (m, 2H), 4.32 (m, 2H), 6.08 & 6.20 (bd, 1H), 7.20 (m, 1H), 7.31 (s, 1H), 7.82 (t, 1H, J=1.8 Hz), 8.14 (m, 1H), 8.19 (s, 1H), 8.55 (s, 1H), 8.81 (s, 1H); MS (ESI) m/z=510 (MH⁺).

### Example 518

### [3-chloro-6-(furan-3-yl)-8-(trifluoromethyl)imidazo[1,2-a]pyridin-2-yl][4-(pyrimidin-5-yl)-3,6-dihydropyridin-1(2H)-yl]methanone (Compound 618)

Prepared using similar procedure as in Example 516 (compound 616). ¹H NMR (d₆-DMSO, 300 MHz) δ 2.63 (m, 2H), 3.90 (m, 2H), 4.42 (m, 2H), 6.39 & 6.51 (bd, 1H), 7.31 (s, 1H), 7.82 (t, 1H, J=1.8 Hz), 8.19 (m, 1H), 8.55 (s, 1H), 8.81 (s, 1H), 8.91 (s, 1H), 9.06 (d, 1H, J=4.5 Hz); MS (ESI) m/z=475 (MH⁺).

### Example 519

### [3-chloro-6-(furan-3-yl)-8-(trifluoromethyl)imidazo[1,2-a]pyridin-2-yl][4-(1,6-dihydropyrimidin-5-yl)-3,6-dihydropyridin-1(2H)-yl]methanone (Compound 619)

To a stirred solution of [3-chloro-6-(furan-3-yl)-8-(trifluoromethyl)imidazo[1,2-a]pyridin-2-yl][4-(pyrimidin-5-yl)-3,6-dihydropyridin-1(2H)-yl]methanone (Example 518, compound 618) (80 mg, 0.17 mmol) in TFA (1 mL) was added Et₃SiH (0.27 mL, 1.7 mmol). The mixture was heated at 70° C. for 16 hours. After evaporation of the solvent, the crude product was purified by reverse phase HPLC to afford the title compound (25%). ¹H NMR (d₆-DMSO, 300 MHz) δ 2.36 (m, 2H), 3.79 (m, 2H), 4.18 (m, 1H), 4.25 (m, 2H), 4.38 (m, 1H), 5.52 & 5.66 (bs, 1H), 6.39 (m, 1H), 7.32 (s, 1H), 7.83 (s, 1H), 8.18 (s, 1H), 8.20 (s, 1H), 8.55 (s, 1H), 8.82 (s, 1H), 10.40 (s, 1H); MS (ESI) m/z=477 (MH⁺).

### Example 520

### [3-chloro-6-(furan-3-yl)-8-(trifluoromethyl)imidazo[1,2-a]pyridin-2-yl][4-(5-methyl-1H-pyrazol-4-yl)-3,6-dihydropyridin-1(2H)-yl]methanone (Compound 620)

Prepared using similar procedure as in Example 516 (compound 616). ¹H NMR (d₆-DMSO, 300 MHz) δ 2.26 (m, 2H), 2.43 (s, 3H), 3.80 (m, 2H), 4.26 (m, 2H), 5.66 & 5.81 (bd, 1H), 7.27 (s, 11H), 7.66 (m, 1H), 7.78 (s, 1H), 8.15 (s, 1H), 8.50 (s, 1H), 8.76 (s, 1H), MS (ESI) m/z=477 (MH⁺).

### General Procedure for Examples 521, 523-529

A mixture of boronate ester/boronic acid (0.10 mmol), 6-bromo-3-chloro-8-trifluoromethyl-imidazo[1,2-a]pyridin-2-yl)-[3-(3-fluoro-phenyl)-pyrrolidin-1-yl]-methanone (0.25 mmol) and Pd(dppf)Cl₂.CH₂Cl₂ (4 mg, 0.005 mmol) in 3M K₃PO₄ (0.5 mL) and 1,4-dioxane (1.2 mL) was heated at 100° C. for 12 hours. The mixture was diluted with EtOAc (25 mL) and washed with saturated aqueous NaHCO₃ (10 mL), and brine (10 mL). The extracts were dried (Na₂SO₄), filtered and concentrated. Preparative HPLC purification (30-100% ACN gradient) of the crude product gave the final product (˜35% yield) as a white powder.

### Example 521

### (3-Chloro-6-pyrimidin-5-yl-8-trifluoromethyl-imidao [1,2-a]pyridin-2-yl)-[3-(3-fluoro-phenyl)-pyrrolidin-1-yl]-methanone (Compound 621)

MS (ESI) m/z=491 (MH⁺).

### Example 522

### [3-Chloro-6-(1,6-dihydro-pyrimidin-5-yl)-8-trifluoromethyl-imidazo[1,2-a]pyridin-2-yl]-[3-(3-fluoro-phenyl)-pyrrolidin-1-yl]-methanone (Compound 622)

(3-Chloro-6-pyrimidin-5-yl-8-trifluoromethyl-imidazo[1,2-a]pyridin-2-yl)-[3-(3-fluoro-phenyl)-pyrrolidin-1-yl]-methanone was treated with triethylsilane similar to Example 519 (compound 619). MS (ESI) m/z=493 (MH⁺).

### Example 523

### [3-Chloro-6-(2-dimethylamino-pyrimidin-5-yl)-8-trifluoromethyl-imidazo[1,2-a]pyridin-2-yl]-[3-(3-fluoro-phenyl)-pyrrolidin-1-yl]-methanone (Compound 623)

Prepared using general procedure described in Example 521 (compound 621). MS (ESI) m/z=534 (MH⁺).

### Example 524

### [3-Chloro-6-(1-methyl-1H-pyrazol-4-yl)-8-trifluoromethyl-imidazo[1,2-a]pyridin-2-yl]-[3-(3-fluoro-phenyl)-pyrrolidin-1-yl]-methanone (Compound 624)

Prepared using general procedure described in Example 521 (compound 621).

¹H NMR (d₆-DMSO, 300 MHz) δ 2.05 (m, 1H), 2.26 (m, 1H), 3.82 (s, 3H), 3.31-4.28 (m, 5H), 7.09 (m, 1H), 7.19 (m, 2H), 7.36 (m, 1H), 8.14 (d, 1H, J=8.1 Hz), 8.17 (d, 1H, J=3.3 Hz), 8.48 (d, 1H, J=4.2 Hz), 8.79 (d, 1H, J=4.2 Hz); MS (ESI) m/z=492 (MH⁺).

### Example 525

### [3-Chloro-6-(3-methyl-1H-pyrazol-4-yl)-8-trifluorom ethyl-imidazo[1,2-a]pyridin-2-yl]-[3-(3-fluoro-phenyl)-pyrrolidin-1-yl]-methanone (Compound 625)

Prepared using general procedure described in Example 521 (compound 621).

¹H NMR (d₆-DMSO, 300 MHz) δ 2.07 (m, 1H), 2.30 (m, 1H), 3.31 (s, 3H), 3.31-4.28 (m, 5H), 7.05 (m, 1H), 7.21 (m, 2H), 7.37 (m, 1H), 7.91 (bs, 1H), 7.99 (m, 1H), 8.30 (bs, 1H), 8.49 (d, 1H, J=5.1 Hz); MS (ESI) m/z=492 (MHt).

### Example 526

### [3-Chloro-6-(2-morpholin-4-yl-thiazol-4-yl)-8-trifluoromethyl-imidazo[1,2-a]pyridin-2-yl]-[3-(3-fluoro-phenyl)-pyrrolidin-1-yl]-methanone (Compound 626)

Prepared using general procedure described in Example 521 (compound 621). MS (ESI) m/z=581 (MH⁺).

### Example 527

### N-(3-{3-Chloro-2-[3-(3-fluoro-phenyl)-pyrrolidine-1-carbonyl]-8-trifluoromethyl-imidazo[1,2-a]pyridin-6-yl}-pyridin-2-yl)-2,2-dimethyl-propionamide (Compound 627)

Prepared using general procedure described in Example 521 (compound 621). MS (ESI) m/z=589 (MH⁺).

### Example 528

### [6-(2-Amino-pyridin-3-yl)-3-chloro-8-trifluoromethyl-imidazo[1,2-a]pyridin-2-yl]-[3-(3-fluoro-phenyl)-pyrrolidin-1-yl]-methanone (Compound 628)

Prepared using general procedure described in Example 521 (compound 621) to give (3-{3-chloro-2-[3-(3-fluoro-phenyl)-pyrrolidine-1-carbonyl]-8-trifluoromethyl-imidazo[1,2-a]pyridin-6-yl}-pyridin-2-yl)-carbamic acid tert-butyl ester which was treated with acid to give the title compound as a light brown solid (45%) after purification. MS (ESI) m/z=504 (MH⁺).

### Example 529

### [3-chloro-6-(1,2,3,6-tetrahydropyridin-4-yl)-8-(trifluoromethyl)imidazo[1,2-a]pyridin-2-yl][3-(3-fluorophenyl)pyrrolidin-1-yl]methanone (Compound 629)

Prepared using general procedure described in Example 521 (compound 621).

¹H NMR (d₆-DMSO, 300 MHz) [symbol] 2.08 (m, 1H), 2.34 (m, 1H), 2.76 (m, 2H), 3.46 (m, 1H), 3.69 (m, 2H), 3.78 (m, 2.5H), 4.06 (m, 3H), 4.22 (m, 0.5H), 6.52 (m, 1H), 7.01 (m, 1H), 7.17 (m, 2H), 7.38 (m, 1H), 8.06 (d, 1H, J=7.5 Hz), 8.45 (d, 1H, J=4.8 Hz), 8.80 (d, 1H); MS (ESI) m/z=494 (MH⁺).

### Example 530

### 1-{4-[3-Chloro-2-{[3-(3-fluorophenyl)pyrrolidin-1-yl]carbonyl}-8-(trifluoromethyl)imidazo[1,2-a]pyridin-6-yl]-3,6-dihydropyridin-1(2H)-yl}ethanone (Compound 630)

To a solution of [3-chloro-6-(1,2,3,6-tetrahydropyridin-4-yl)-8-(trifluoromethyl)imidazo[1,2-a]pyridin-2-yl][3-(3-fluorophenyl)pyrrolidin-1-yl]-methanone (40 mg, 0.08 mmol) in THF (2 mL) at 0° C. was added N,N-diisopropylethylamine (0.04 mL, 0.24 mmol). After 15 min, acetyl chloride (0.02 mL, 0.24 mmol) was added and the solution was stirred for 10 hours at room temperature. The mixture was carefully poured into ice-water (2 mL) to give a white precipitate which was filtered and dried under high vacuum to give 1-{4-[3-chloro-2-{[3-(3-fluorophenyl)pyrrolidin-1-yl]carbonyl}-8-(trifluoromethyl)imidazo[1,2-a]pyridin-6-yl]-3,6-dihydropyridin-1(2H)-yl}ethanone (80%) as a light yellow solid. ¹H NMR (d₆-DMSO, 300 MHz) δ2.27 (m, 2H), 2.59 (m, 1H), 2.64 (m, 2H), 3.13 (s, 3H), 3.45 (m, 1H), 3.65 (m, 3H), 3.75 (m, 1H), 4.03 (m, 1H), 4.17 (m, 2H), 7.06 (m, 1H), 7.19 (m, 2H), 7.37 (m, 1H), 8.04 (m, 1H), 8.41 (d, 1H); MS (ESI) m/z=535 (MH⁺).

### Example 531

### {3-chloro-6-[1-(methylsulfonyl)-1,2,3,6-tetrahydropyridin-4-yl]-8-(trifluoromethyl)imidazo[1,2-a]pyridin-2-yl}[3-(3-fluorophenyl)pyrrolidin-1-yl]methanone (Compound 631)

To a solution of [3-chloro-6-(1,2,3,6-tetrahydropyridin-4-yl)-8-(trifluoromethyl)imidazo[1,2-a]pyridin-2-yl][3-(3-fluorophenyl)pyrrolidin-1-yl]methanone (35 mg, 0.07 mmol) in THF (2 mL) at 0° C. was added N,N-diisopropylethylamine (0.04 mL, 0.21 mmol). After 15 min, mesyl chloride (0.02 mL, 0.24 mmol) was added and the solution was stirred for 10 hours at room temperature. The mixture was diluted with EtOAc (15 mL) and washed successively with 2N HCl (2×2 mL), saturated aqueous NaHCO₃ (5 mL), and brine (50 mL). The organic layer was concentrated, and the crude material was purified by preparative preparative TLC (6% MeOH/DCM gradient) to give the title compound as a light brown solid (40%). MS (ESI) m/z=572 (MH⁺).

### Example 532

### 3-Chloro-2-[3-(3-fluoro-phenyl)-pyrrolidine-1-carbonyl]-8-trifluoromethyl-imidazo[1,2-a]pyridine-6-carboxylic Acid Butyl Ester (Compound 632)

A mixture of (6-bromo-3-chloro-8-trifluoromethyl-imidazo[1,2-a]pyridin-2-yl)-[3-(3-fluoro-phenyl)-pyrrolidin-1-yl]-methanone (0.07 mg, 0.14 mmol), N,N-diisopropylethylamine (0.06 mL, 0.35 mmol), Pd(dppf)Cl₂CH₂ Cl₂ (5 mg, 0.007 mmol) in n-BuOH (3 mL) was degassed and stirred at 90° C. for 12 hours under CO atm (balloon). The mixture was concentrated and purified by preparative TLC (5% MeOH/DCM) to give the title compound as a white solid (41%). MS (ESI) m/z=513 (MH⁺).

### Example 533

### [3-Chloro-6-(5-chloro-furan-3-yl)-8-trifluoromethyl-imidazo[1,2-a]pyridin-2-yl]-[3-(3-fluoro-phenyl)-pyrrolidin-1-yl]-methanone (Compound 633)

### Step 1: 3-Chloro-6-(5-chloro-furan-3-yl)-8-trifluoromethyl-imidazo[1,2-a]pyridine-2-carbonyl Chloride

A solution of 3-chloro-6-furan-3-yl-8-trifluoromethyl-imidazo[1,2-a]pyridine-2-carboxylic acid (0.25 g, 0.7 mmol) in thionyl chloride (2 mL) was heated at 70° C. for 12 hours. The reaction mixture was concentrated in high vacuum and co-evaporated with toluene (2×10 mL) to give the crude product (0.20) which was used in the next step without further purification.

### Step 2: [3-Chloro-6-(5-chloro-furan-3-yl)-8-trifluoromethyl-imidazo[1,2-a]pyridin-2-yl]-[3-(3-fluoro-phenyl)-pyrrolidin-1-yl]-methanone (Compound 633)

To a solution of 3-chloro-6-(5-chloro-furan-3-yl)-8-trifluoromethyl-imidazo[1,2-a]pyridine-2-carbonyl chloride (0.20 g, 0.6 mmol) in THF (1 mL) was added a solution of 3-(3-fluoro-phenyl)-pyrrolidine HCl salt (0.24 g, 1.2 mmol) and N,N-diisopropylethylamine (0.2 mL, 1.2 mmol) in THF (1 mL). The mixture was allowed to stir at room temperature for 12 hours. The mixture was diluted with EtOAc (20 mL) and washed with aqueous HCl (10%, 2 mL) and brine (2×10 mL). The extracts were dried (Na₂SO₄), filtered and concentrated and the crude material was purified by preparative HPLC (30-100% ACN gradient) to give the title compound (25%) as a white powder. ¹H NMR (d₆-DMSO, 300 MHz) δ 2.04 (m, 1H), 2.25 (m, 1H), 3.48-3.69 (m, 3H), 4.00 (m, 1.5H), 4.19 (m, 0.5H), 7.02 (m, 1H), 7.14 (m, 2H), 7.31 (m, 2H), 7.86 (dd, 1H, J=2.7, 2.1 Hz), 8.07 (d, 1H, J=7.1 Hz), 8.75 (d, 1H, J=5.7 Hz); MS (ESI) m/z=513 (MH⁺).

### Example 534

### [3-chloro-6-(furan-3-yl)-8-(trifluoromethyl)imidazo[1,2-a]pyridin-2-yl][3-(1,3-thiazol-4-yl)-8-azabicyclo[3.2.1]oct-2-en-8-yl]methanone (Compound 634)

### Step 1: 3-Trifluoromethanesulfonyloxy-8-aza-bicyclo[3.2.1]oct-2-ene-8-carboxylic Acid Tert-butyl Ester

A solution of 3-oxo-8-aza-bicyclo[3.2.1]octane-8-carboxylic acid tert-butyl ester (0.7 g, 3.1 mmol) in THF (10 mL) was slowly added to a stirring solution of LDA (2M, 6.2 mmol) in THF (10 mL) at −78° C. After 10 min, a solution of N-phenyl bis(trifluoromethanesulfonimide) (2.14 g, 6.2 mmol) in THF (10 mL) was slowly added. After 30 min, the cooling bath was removed and reaction mixture was allowed to warm to room temperature over the course of 1.5 hours. The mixture was cooled to 0° C., quenched with saturated aqueous NaHCO₃ (30 mL), and extracted with ether (200 mL). The organic layer was washed with 5% citric acid (40 mL), 1M NaOH (4×40 mL), H₂O (2×40 mL), brine (40 mL), dried (MgSO₄), concentrated on silica and flash column chromatography (15-50% EtOAc/n-hexane gradient) afforded 3-trifluoromethanesulfonyloxy-8-aza-bicyclo[3.2.1]oct-2-ene-8-carboxylic acid tert-butyl ester (90%) as brown oil. MS (ESI) m/z=378 (MNa⁺).

### Step 2: 3-(4,4,5,5-Tetramethyl-[1,3,2]dioxa borolan-2-yl)-8-aza-bicyclo[3.2.1]oct-2-ene-8-carboxylic Acid Tert-butyl Ester

3-Trifluoromethanesulfonyloxy-8-aza-bicyclo[3.2.1]oct-2-ene-8-carboxylic acid tert-butyl ester (0.3 g, 0.8 mmol) was dissolved in 1,4-dioxane (5 mL) and added under N₂ (g) to a degassed mixture of potassium acetate (0.23 g, 2.4 mmol), Pd(dppf)Cl₂*CH₂Cl₂ (6 mg, 0.08 mmol), dppf (13 mg, 0.024 mmol), bis-pinacolato diborane (0.3 g, 2.32 mmol) and the reaction mixture heated at 80° C. overnight. The reaction mixture was concentrated and purified by flash column chromatography (15-50% EtOAc/n-hexane gradient) which afforded 3-(4,4,5,5-tetramethyl-[1,3,2]dioxa borolan-2-yl)-8-aza-bicyclo[3.2.1]oct-2-ene-8-carboxylic acid tert-butyl ester (90%) as white viscous material. MS (ESI) m/z=358 (MNa⁺).

### Step 3: 3-Thiazol-4-yl-8-aza-bicyclo[3.2.1]oct-2-ene-8-carboxylic Acid Tert-butyl Ester

Prepared using Suzuki coupling protocol as in Example 521.

¹H NMR (d₆-DMSO, 300 MHz) δ 1.40-2.30 (m, 5H), 3.15 (m, 1H), 4.51 (m, 2H), 6.94 (d, 1H, J=5.1 Hz), 7.03 (d, 1H, J=4.8 Hz), δ 8.74 (d, 1H, J=5.1); MS (ESI) m/z=237 (MH⁺-^{t}Bu).

### Step 4: 3-Thiazol-4-yl-8-aza-bicyclo [3.2.1]oct-2-ene

3-Thiazol-4-yl-8-aza-bicyclo[3.2.1]oct-2-ene-8-carboxylic acid tert-butyl ester underwent HCl deprotection to give 3-thiazol-4-yl-8-aza-bicyclo[3.2.1]oct-2-ene; MS (ESI) m/z=193 (MH⁺).

### Step 5: [3-chloro-6-(furan-3-yl)-8-(trifluoromethyl)imidazo[1,2-a]pyridin-2-yl][3-(1,3-thiazol-4-yl)-8-azabicyclo[3.2.1]oct-2-en-8-yl]methanone (Compound 634)

A solution of 3-thiazol-4-yl-8-aza-bicyclo[3.2.1]oct-2-ene (60 mg, 0.3 mmol), EDC (0.11 g, 0.6 mmol), HOAT (0.4 mmol), N,N-diisopropylethylamine (0.08 mg, 0.6 mmol) and 3-chloro-6-furan-2-yl-8-trifluoromethyl-imidazo[1,2-a]pyridine-2-carboxylic acid (0.14 g, 0.45 mmol) in DMF (2 mL) was stirred at room temperature for 4 hours. The reaction mixture was concentrated and washed with saturated aqueous NaHCO₃ (30 mL), brine (30 mL) and dried (MgSO₄). The crude material was purified by preparative TLC (80% EtOAc/n-hexane) which afforded the title compound (25%) as a white solid. ¹H NMR (d₆-DMSO, 300 MHz) δ 1.70 (m, 1H), 1.96 (m, 2H), 2.17 (m, 1H), 2.40 (m, 1H), 3.08 (m, 1H), 4.90 (m, 1H), 5.20 (m, 1H), 6.91 (m, 1H), 7.26 (s, 1H), 7.50 (dd, 1H, J=1.8, 17.1 Hz), 7.78 (d, 1H, J=1.5 Hz), 8.15 (s, 1H), 8.49 (s, 1H), 8.74 (d, 1H, J=5.7), 9.01 (dd, 1H, J=1.8, 8.4 Hz); MS (ESI) m/z=506 (MH⁺).

### Example 535

### 2-(1-{[3-chloro-6-(furan-3-yl)-8-(trifluoromethyl)imidazo[1,2-a]pyridin-2-yl]carbonyl}-1,2,3,6-tetrahydropyridin-4-yl)-3,6-difluorobenzonitrile (Compound 635)

Prepared using similar procedure as in Example 534, Step 3, 4 and 5.

¹H NMR (d₆-DMSO, 300 MHz) δ 2.50 (m, 2H), 3.89 (m, 2H), 4.36 (m, 2H), 6.05 & 6.18 (bd, 1H), 7.33 (m, 1H), 7.56 (m, 1H), 7.72 (m, 1H), 7.84 (s, 1H), 8.21 (s, 1H), 8.56 (s, 1H), 8.84 (s, 1H); MS (ESI) m/z=510 (MH⁺).

### Example 536

### [3-(3-Fluoro-phenyl)-pyrrolidin-1-yl]-(6-furan-3-yl-8-methyl-imidazo[1,2-a]pyridin-2-yl)-methanone

6-Bromo-8-methyl-imidazo[1,2-a]pyridine-2-carboxylic acid methyl ester was prepared by reacting 5-bromo-3-methyl-1,2-dihydro-pyridin-2-ylamine with methyl bromopyruvate in DMF at 50° C. 6-Bromo-8-methyl-imidazo[1,2-a]pyridine-2-carboxylic acid methyl ester was then converted to [3-(3-fluoro-phenyl)-pyrrolidin-1-yl]-(6-furan-3-yl-8-methyl-imidazo[1,2-a]pyridin-2-yl)-methanone following similar procedure as in Example 151. MS (ESI) m/z=391 (MH⁺).

### Example 537

### N-{[2-{[3-(3-fluorophenyl)pyrrolidin-1-yl]carbonyl}-6-(furan-3-yl)-8-(trifluoromethyl)imidazo[1,2-a]pyridin-5-yl]methyl}acetamide (Compound 637)

### Step 1: (5-Bromo-3-trifluoromethyl-pyridin-2-yl)-carbamate Di-tert-butyl Ester

DMAP (8.90 g, 72.82 mmol) was slowly added to a stirring solution of di-tert-butyl dicarbonate (61.13 g, 280.08 mmol) and 5-bromo-3-trifluoromethyl-pyridin-2-ylamine (13.5 g, 56.02 mmol) in acetone (300 mL) at room temperature. The mixture was heated to 65° C. and stirred for 4 days, cooled to room temperature, filtered, concentrated on silica and flash column chromatography [EtOAc/hexane 3:7 v/v)] to afford (5-bromo-3-trifluoromethyl-pyridin-2-yl)-carbamate di-tert-butyl ester (23.5 g, 95.1%) as a white crystalline solid. ¹HNMR (CDCl₃, 300 MHz) δ 8.77 (s, 1H), 8.15 (s, 1H), 1.38 (s, 18H). MS (ESI) m/z=287 (M⁺-boc, -^{t}Bu).

### Step 2: (5-Bromo-1-hydroxy-3-trifluoromethyl-pyridin-2-yl)-carbamate Di-tert-butyl Ester

Trifluoroacetic anhydride (12.79 mL, 92.01 mmol) was slowly added to a stirring solution of (5-bromo-3-trifluoromethyl-pyridin-2-yl)-carbamate di-tert-butyl ester (20.3 g, 46.01 mmol) and urea hydrogen peroxide complex (8.66 g, 92.01 mmol) in DCM (300 mL) at 0° C. and the mixture was stirred for 2 hours at 0° C. The mixture was allowed to warm to room temperature over the course of 2 hours. The reaction mixture was quenched with 1M Na₂S₂O₃ (60 mL), stirred for 20 min, then 5% HCl (50 mL) added, stirred for 20 min, and the organic layer was collected. The aqueous layer was extracted with DCM (100 mL), the combined organic layer was dried (MgSO₄), filtered and concentrated on silica. Flash column chromatography (10-30% EtOAc/hexane gradient) of the crude afforded (5-bromo-1-hydroxy-3-trifluoromethyl-pyridin-2-yl)-carbamate di-tert-butyl ester (10.2 g, 48.5%) as a pale white solid. ¹HNMR (d₆-DMSO, 300 MHz) δ 9.17 (s, 1H), 8.15 (s, 1H), 1.32 (s, 18H). MS (ESI) m/z=303 (MH⁺-boc-^{t}Bu).

### Step 3: (5-Bromo-6-cyano-3-trifluoromethyl-pyridin-2-yl)-carbamate Di-tert-butyl Ester

A stirred solution of TMSCN (8.21 mL, 65.61 mmol), TEA (9.14 mL, 65.61 mmol), and (5-bromo-1-hydroxy-3-trifluoromethyl-pyridin-2-yl)-carbamate di-tert-butyl ester (10 g, 21.87 mmol) in ACN (300 mL) was heated at 75° C. overnight. The reaction mixture was concentrated on silica and flash column chromatography (5-100% EtOAc/hexane gradient) afforded (5-bromo-6-cyano-3-trifluoromethyl-pyridin-2-yl)-carbamate di-tert-butyl ester (4.53 g, 44.4%) as a white solid. ¹HNMR (d₆-DMSO, 300 MHz) δ 9.07 (s, 1H), 1.35 (s, 18H). MS (ESI) m/z=312 (M⁺-boc, -tBu).

### Step 4: (6-Cyano-5-furan-3-yl-3-trifluoromethyl-pyridin-2-yl)-carbamic Acid Di-tert-butyl Ester

(5-Bromo-6-cyano-3-trifluoromethyl-pyridin-2-yl)-carbamate di-tert-butyl ester and 3-furanboronic acid reacted under standard Suzuki conditions to give (6-cyano-5-furan-3-yl-3-trifluoromethyl-pyridin-2-yl)-carbamic acid di-tert-butyl ester. MS (ESI) m/z=298 (M⁺-boc,-tBu).

### Step 5: (6-Aminomethyl-5-furan-3-yl-3-trifluoromethyl-pyridin-2-yl)-carbamic Acid Di-tert-butyl Ester

A suspension of (6-cyano-5-furan-3-yl-3-trifluoromethyl-pyridin-2-yl)-carbamic acid di-tert-butyl ester (1.7 g, 3.7 mmol) and Raney®-nickel (wet 50 mg) in EtOH was stirred under H₂ at 65 psi for 10 days. The catalyst was carefully filtered through Celite and the solvent concentrated under reduced pressure to give (6-aminomethyl-5-furan-3-yl-3-trifluoromethyl-pyridin-2-yl)-carbamic acid di-tert-butyl ester (99%) which was used for the next step without further purification. MS (ESI) m/z=458 (MH⁺).

### Step 6: [6-(Acetylamino-methyl)-5-furan-3-yl-3-trifluoromethyl-pyridin-2-yl]-carbamic Acid Di-tert-butyl Ester

To a solution of (6-aminomethyl-5-furan-3-yl-3-trifluoromethyl-pyridin⁻²-yl)-carbamic acid di-tert-butyl ester (0.5 g, 1 mmol) in THF (3 mL) was added Et₃N (0.4 mL, 3 mmol). After 15 min, acetyl chloride (0.23 mL, 3 mmol) was added and the solution was stirred for 10 hours at room temperature. The reaction mixture was concentrated and the residue was acidified with 10% HCl and extrated with EtOAc (2×20 mL). The organic layer was washed with brine (50 mL), dried (MgSO₄), filtered and concentrated. The crude was subjected to flash column chromatorgarphy [EtOAc/n-hexane (1:1 v/v)] to afford the compound [6-(acetylamino-methyl)-5-furan-3-yl-3-trifluoromethyl-pyridin-2-yl]-carbamic acid di-tert-butyl ester (60%) as brown solid. MS (ESI) m/z=500 (MH⁺).

### Step 7: N-(6-Amino-3-furan-3-yl-5-trifluoromethyl-pyridin-2-ylmethyl)-acetamide

4M HCl solution in 1,4-dioxane (10 eq) was added to a stirring solution of [6-(acetylamino-methyl)-5-furan-3-yl-3-trifluoromethyl-pyridin-2-yl]-carbamic acid di-tert-butyl ester (0.25 g, 0.5 mmol) in THF (5 mL) and was stirred at 60° C. for 12 hours. The reaction mixture was concentrated to give N-(6-amino-3-furan-3-yl-5-trifluoromethyl-pyridin-2-ylmethyl)-acetamide as HCl salt (˜90%). ¹HNMR (d₆-DMSO, 300 MHz) δ 8.12 (s, 1H), 7.85 (s, 1H), 7.74 (m, 2H), 6.77 (s, 1H), 4.93 (bs, 2H), 4.27 (d, 2H, J=4.5 Hz), 2.47 (s, 3H). MS (ESI) m/z=300 (MH⁺).

### Step 8: 5-(Acetylamino-methyl)-6-furan-3-yl-8-trifluoromethyl-imidazo[1,2-a]pyridine-2-carboxylic Acid Methyl Ester

N-(6-Amino-3-furan-3-yl-5-trifluoromethyl-pyridin-2-ylmethyl)-acetamide reacted with methyl bromopyruvate to give 5-(acetylamino-methyl)-6-furan-3-yl-8-trifluoromethyl-imidazo[1,2-a]pyridine-2-carboxylic acid methyl ester. (ESI) m/z=382 (MH⁺).

### Step 9: 5-(Acetylamino-methyl)-6-furan-3-yl-8-trifluoromethyl-imidazo[1,2-a]pyridine-2-carboxylic Acid

5-(Acetylamino-methyl)-6-furan-3-yl-8-trifluoromethyl-imidazo[1,2-a]pyridine-2-carboxylic acid methyl ester was saponified using sodium hydroxide to give 5-(acetylamino-methyl)-6-furan-3-yl-8-trifluoromethyl-imidazo[1,2-a]pyridine-2-carboxylic acid. MS (ESI) m/z=368 (MH⁺).

### Step 10: N-{[2-{[3-(3-fluorophenyl)pyrrolidin-1-yl]carbonyl}-6-(furan-3-yl)-8-(trifluoromethyl)imidazo[1,2-a]pyridin-5-yl]methyl}acetamide (compound 637)

Prepared using standard HATU coupling of the above acid. ¹H NMR (d₆-DMSO, 300 MHz) δ 1.95 (s, 3H) 2.07 (m, 1H), 2.44 (m, 1H), 3.57-4.15 (m, 5H), 4.88 (s, 2H), 6.82 (s, 1H), 7.05 (m, 1H), 7.22 (m, 2H), 7.40 (m, 2H), 7.73 (s, 1H), 7.94 (s, 1H), 8.10 (m, 1H), 8.88 (m, 1H); MS (ESI) m/z=515 (MH⁺).

### Example 538

### [3-(3-Fluoro-phenyl)-pyrrolidin-1-yl]-(6-furan-3-yl-8-trifluoromethyl-imidazo[1,2-a]pyridin-2-yl)-methanone (Compound 638)

Prepared using standard HATU coupling. ¹H NMR (d₆-DMSO, 300 MHz) δ 2.07 (m, 1H), 2.40 (m, 1H), 3.38-4.05 (m, 4H), 4.33 (m, 0.5H), 4.53 (m, 0.5H), 7.02 (m, 2H), 7.19 (m, 2H), 7.35 (m, 1H), 7.82 (m, 1H), 8.08 (d, 1H, J=4.8 Hz), 8.42 (m, 2H), 9.13 (d, 1H, J=5.1 Hz); MS (ESI) m/z=445. (MH⁺).

### Example 539

### [3-(3-Fluoro-phenyl)-pyrrolidin-1-yl]-(6-phenyl-8-trifluoromethyl-imidazo[1,2-a]pyridin-2-yl)-methanone (Compound 639)

Prepared using standard HATU coupling. ¹H NMR (d₆-DMSO, 300 MHz) δ 2.04 (m, 1H), 2.29 (m, 1H), 3.47 (m, 2H), 3.74 (m, 1H), 4.01 (m, 1H), 4.29 (m, 0.5H), 4.52 (m, 0.5H), 7.02 (m, 1H), 7.15 (m, 2H), 7.37 (m, 2H), 7.46 (m, 2H), 7.72 (m, 2H), 8.01 (d, 1H, J=7.2 Hz), 8.45 (d, 1H, J=2.7 Hz), 9.12 (d, 1H, J=4.5 Hz); MS (ESI) m/z=454 (MH⁺).

### Example 540

### (3-Bromo-6-phenyl-8-trifluoromethyl-imidazo[1,2-a]pyridin-2-yl)-[3-(3-fluoro-phenyl)-pyrrolidin-1-yl]-methanone (Compound 640)

### Step 1: 3-Bromo-6-phenyl-8-trifluoromethyl-imidazo[1,2-a]pyridine-2-carboxylic Acid

A mixture of 6-phenyl-8-trifluoromethyl-imidazo[1,2-a]pyridine-2-carboxylic acid (0.15 g, 0.4 mmol) and N-bromosuccinimide (90 mg, 0.51 mmol) was stirred at room temperature in DMF (3 mL) for 12 hours. The mixture was diluted with EtOAc (10 mL) and washed with water (10 mL), 1M sodium thiosulfate solution (10 mL), and brine (10 mL). The filtrate was dried (Na₂SO₄), filtered and concentrated to give 3-bromo-6-phenyl-8-trifluoromethyl-imidazo[1,2-a]pyridine-2-carboxylic acid (30%) as a brown solid. MS (ESI) m/z=386 (MH⁺).

### Step 2: (3-Bromo-6-phenyl-8-trifluoromethyl-imidazo[1,2-a]pyridin-2-yl)-[3-(3-fluoro-phenyl)-pyrrolidin-1-yl]-methanone

Prepared using standard HATU coupling of the above acid. ¹H NMR (d₆-DMSO, 300 MHz) δ 2.12 (m, 1H), 2.31 (m, 1H), 3.49 (m, 1.5H), 3.66 (m, 1H), 3.78 (m, 1H), 4.07 (m, 1H), 4.22 (m, 0.5H), 7.07 (m, 1H), 7.21 (m, 2H), 7.34 (m, 1H), 7.51 (m, 3H), 7.84 (m, 2H), 8.17 (d, 1H, J=7.2 Hz), 8.71 (d, 1H, J=5.4 Hz); MS (ESI) m/z=524 (MH⁺).

### Example 541

### (3-Chloro-6-phenyl-8-trifluoromethyl-imidazo[1,2-a]pyridin-2-yl)-[3-(3-fluoro-phenyl)-pyrrolidin-1-yl]-methanone (Compound 641)

Prepared using standard HATU coupling. ¹H NMR (d₆-DMSO, 300 MHz) δ 2.02 (m, 1H), 2.24 (m, 1H), 3.41 (m, 2H), 3.69 (m, 1.5H), 4.00 (m, 1H), 4.21 (m, 0.5H), 7.02 (m, 3H), 7.29 (m, 1H), 7.41 (m, 3H), 7.76 (m, 2H), 8.08 (d, 1H, J=7.2 Hz), 8.69 (s, 1H); MS (ESI) m/z=524 (MH⁺).

### Example 542

### 1-{3-Chloro-2-[3-(3-fluoro-phenyl)-pyrrolidine-1-carbonyl]-8-trifluoromethyl-imidazo[1,2-a]pyridin-6-yl}-ethanone (Compound 642)

### Step 1: [3-Chloro-6-(1-ethoxy-vinyl)-8-trifluoromethyl-imidazo[1,2-a]pyridin-2-yl]-[3-(3-fluoro-phenyl)-pyrrolidin-1-yl]-methanone

To a solution (6-Bromo-3-chloro-8-trifluoromethyl-imidazo[1,2-a]pyridin-2-yl)-[3-(3-fluoro-phenyl)-pyrrolidin-1-yl]-methanone (0.3 g, 0.6 mmol) in DMF (3 mL) were added (1-ethoxyviny I)trimethyltin (0.1 g, 1.2 mmol), Et₃N (0.17 mL, 1.2 mmol), p-O-tolylphosphine (0.18 g, 0.6 mmol) and Pd(OAc)₂ (13.5 mg, 0.06 mmol). The resulting orange suspension was degassed and was stirred at 90° C. for 16 hours. The black suspension was concentrated and diluted with DCM (10 mL) and washed with a 5% KF solution, water, and brine. The organic layer was dried (MgSO₄) and concentrated to a yellow oil. Preparative TLC (60% EtOAc/n-hexane) of the crude product yielded [3-chloro-6-(1-ethoxy-vinyl)-8-trifluoromethyl-imidazo[1,2-a]pyridin-2-yl]-[3-(3-fluoro-phenyl)-pyrrolidin-1-yl]-methanone (55%) as a light brown solid. MS (ESI) m/z=482 (MH⁺).

### Step: 2: 1-{3-Chloro-2-[3-(3-fluoro-phenyl)-pyrrolidine-1-carbonyl]-8-trifluoromethyl-imidazo[1,2-a]pyridin-6-yl}-ethanone (Compound 642)

Aqueous HCl (3M, 0.5 mmol) solution was added to [3-chloro-6-(1-ethoxy-vinyl)-8-trifluoromethyl-imidazo[1,2-a]pyridin-2-yl]-[3-(3-fluoro-phenyl)-pyrrolidin-1-yl]-methanone (120 mg, 0.2 mmol) in THF (1 mL) and was stirred at room temperature for 4 hours. The reaction mixture was concentrated and the crude material was subjected to preparative TLC (6% MeOH/DCM) to give the title compound (65%) as a light brown solid. ¹H NMR (d₆-DMSO, 300 MHz) δ 2.07 (m, 1H), 2.30 (m, 1H), 2.75 (s, 3H), 3.33-4.24 (m, 5H), 7.13 (m, 1H), 7.22 (m, 2H), 7.37 (m, 1H), 8.14 (d, 1H, J=7.8 Hz), 9.20 (d, 1H, J=5.1 z); MS (ESI) m/z=454 (MH⁺).

### Example 543

### 2[6-(2-Amino-thiazol-4-yl)-3-chloro-8-trifluoromethyl-imidazo[1,2-a]pyridin-2-yl]-[3-(3-fluoro-phenyl)-pyrrolidin-1-yl]-methanone (Compound 643)

### Step:1 2-Bromo-1-{3-chloro-8-(1,1-difluoro-ethyl)-2-[3-(3-fluoro-phenyl)-pyrrolidine-1-carbonyl]-imidazo[1,2-a]pyridin-6-yl}-ethanone

A mixture of [3-chloro-6-(1-ethoxy-vinyl)-8-trifluoromethyl-imidazo[1,2-a]pyridin-2-yl]-[3-(3-fluoro-phenyl)-pyrrolidin-1-yl]-methanone (0.1 g, 0.2 mmol) and N-bromosuccinimide (73 mg, 0.4 mmol) was stirred at room temperature in DMF (3 mL) for 12 hours. The mixture was diluted with EtOAc (10 mL) and washed with water (10 mL), 1M sodium thiosulfate solution (10 mL), and brine (10 mL). The filtrate was dried (Na₂SO₄), filtered and concentrated to give 2-bromo-1-{(3-chloro-8-(1,1-difluoro-ethyl)-2-[3-(3-fluoro-phenyl)-pyrrolidine-1-carbonyl]-imidazo[1,2-a]pyridin-6-yl}-ethanone (45%) as a brown solid. MS (ESI) m/z=534 (MH⁺).

### Step: 2 [6-(2-Amino-thiazol-4-yl)-3-chloro-8-trifluoromethyl-imiidazo[1,2-a]pyridin-2-yl]-[3-(3-fluoro-phenyl)-pyrrolidin-1-yl]-methanone (Compound 643)

To a solution of 2-bromo-1-{3-chloro-8-(1,1-difluoro-ethyl)-2-[3-(3-fluoro-phenyl)-pyrrolidine-1-carbonyl]-imidazo[1,2-a]pyridin-6-yl}-ethanone (100 mg, 0.18 mmol) in EtOH (3 mL) was added thiourea (27 mg, 0.36 mmol) and mixture was stirred at room temperature for 12 hours. The reaction mixture was concentrated and subjected to preprative TLC (5% MeOH/DCM) to provide [6-(2-amino-thiazol-4-yl)-3-chloro-8-trifluoromethyl-imidazo[1,2-a]pyridin-2-yl]-[3-(3-fluoro-phenyl)-pyrrolidin-1-yl]-methanone (50%) as light yellow solid. ¹H NMR (d₆-DMSO, 300 MHz) δ 2.06 (m, 1H), 3.26 (m, H), 3.44 (s, 1.5H), 3.55 (m, 0.5H), 3.75 (m, 1H), 3.87 (m, 0.5H), 4.04 (m, 1H), 4.27 (m, 0.5H), 7.04 (m, 1H), 7.16 (m, 2H), 7.36 (m, 3H), 7.50 (d, 1H, J=4.5 Hz), 8.33 (m, 1H), 8.80 (m, 1H); MS (ESI) m/z=510 (MH⁺).

### Example 544

### N-(4-{3-Chloro-2-[3-(3-fluoro-phenyl)-pyrrolidine-1-carbonyl]-8-trifluoromethyl-imidazo[1,2-a]pyridin-6-yl}-thiazol-2-yl)-acetamide (Compound 644)

To a solution of 2 [6-(2-amino-thiazol-4-yl)-3-chloro-8-trifluoromethyl-imidazo[1,2-a]pyridin-2-yl]-[3-(3-fluoro-phenyl)-pyrrolidin-1-yl]-methanone (15 mg, 0.03 mmol) in DMF (2 mL) was added N,N-diisopropylethylamine (0.2 mL, 0.06 mmol) and acetyl chloride (0.007 mL, 0.06 mmol). The solution was stirred for 12 hours at 60° C. The mixture was carefully poured into ice-water (1 mL) and extracted with ethyl acetate (2×5 ml). The organic layer was dried (Na₂SO₄), filtered and concentrated to provide the title compound (75%) as a white solid. ¹H NMR (d₆-DMSO, 300 MHz) δ 2.06 (m, 1H), 2.16 (s, 3H), 2.30 (m, 1H), 3.47-4.29 (m, 5H), 7.07 (m, 1H), 7.19 (m, 2H), 7.36 (m, 1H), 8.08 (d, 1H, J=4.5 Hz), 8.41 (d, 1H, J=8.4 Hz), 8.93 (d, 1H, J=5.1 Hz), 12.44 (s, 1H); MS (ESI) m/z=552 (MH⁺).

### Example 545

### 3-Bromo-8-isopropyl-6-phenyl-imidazo[1,2-a]pyridine-2-carboxylic acid (thiophen-2-ylmethyl)-amide (Compound 645)

### Step 1: 5-Phenyl-pyridin-2-ylamine

5-Phenyl-pyridin-2-ylamine was prepared from Suzuki reaction of 5-bromo-pyridin-2-ylamine and phenylboronic acid. MS (ESI) m/z=171 (MH⁺).

### Step 2: 3-Bromo-5-phenyl-pyridin-2-ylamine

A mixture of 5-phenyl-pyridin-2-ylamine (8 g, 47 mmol) and N-bromosuccinimide (12.46 g, 70 mmol) was stirred at room temperature in DMF (100 mL) for 4 hours. The mixture was concentrated, water (50 mL) was added, and the brown precipitate formed was filtered to yield the first crop of the product. To the rest of the aquous filtrate was added EtOAc (200 mL) and the organic layer was separated, washed with 1M sodium thiosulfate solution (10 mL), and brine (10 mL). The filtrate was dried (Na₂SO₄), filtered and concentrated to give the rest of the title compound (60%) as a brown solid. MS (ESI) m/z=250 (MH⁺).

### Step 3: 8-Bromo-6-phenyl-imidazo[1,2-a]pyridine-2-carboxylic Acid Methyl Ester

A solution of 3-bromo-5-phenyl-pyridin-2-ylamine 1 (4.5 g, 18 mmol) and methyl-3-bromopyruvate (6.5 g, 36 mmol) in DMF (100 mL) was heated at 70° C. for 3 hours. The mixture was concentrated, ice H₂O was added with rapid stirring, and the resulting precipitate filtered, washed with H₂O (4×300 mL), dried under vacuum overnight to give 8-bromo-6-phenyl-imidazo[1,2-a]pyridine-2-carboxylic acid methyl ester (71%) as a brown solid. MS (ESI) m/z=332 (MH⁺).

### Step 4: 8-Isopropenyl-6-phenyl-imidazo[1,2-a]pyridine-2-carboxylic Acid Methyl Ester

A mixture of vinyl boronic acid (1 g, 12 mmol), 8-bromo-6-phenyl-imidazo[1,2-a]pyridine-2-carboxylic acid methyl ester (2 g, 6 mmol) and Pd(PPh₃)₄ (0.7 g, 0.6 mmol) in 3M K₃PO₄ (36 mmol) and 1,4-dioxane (1.2 mL) was heated at 90° C. for 4 hours. The mixture was diluted with EtOAc (25 mL) and washed with saturated aqueous NaHCO₃ (10 mL), and brine (10 mL). The extracts were dried (Na₂SO₄), filtered and concentrated to give crude 8-isopropenyl-6-phenyl-imidazo[1,2-a]pyridine-2-carboxylic acid methyl ester (55%) which was used for the next step without further purification. MS (ESI) m/z=293 (MH⁺).

### Step 5: 8-Isopropyl-6-phenyl-imidazo[1,2-a]pyridine-2-carboxylic Acid Methyl Ester

A suspension of 8-isopropenyl-6-phenyl-imidazo[1,2-a]pyridine-2-carboxylic acid ethyl ester (1.46 g, 5 mmol) and 10% Pd/C (100 mg) was stirred under H₂ at atm pressure in EtOH. After 72 hours, the catalyst was filtered through Celite and the solvent was concentrated under reduced pressure to give 8-isopropyl-6-phenyl-imidazo[1,2-a]pyridine-2-carboxylic acid methyl ester (35%) which was used for the next step without further purification. MS (ESI) m/z=295 (MH⁺).

### Step 6: 8-Isopropyl-6-phenyl-imidazo[1,2-a]pyridine-2-carboxylic Acid

A mixture of 8-isopropyl-6-phenyl-imidazo[1,2-a]pyridine-2-carboxylic acid methyl ester (0.3 g, 1 mmol) and NaOH (2M, 0.6 mmol) was stirred at room temperature in THF/H₂O (3:1 v/v, 100 mL) for 12 hours. The reaction mixture was concentrated and the residue was acidified with 10% HCl and extracted with ethyl acetate (2×20 mL). The organic layer was washed with brine (50 mL), dried (MgSO₄), filtered and concentrated to afford 8-isopropyl-6-phenyl-imidazo[1,2-a]pyridine-2-carboxylic acid (42%) as a light brown solid which was used without further purification. MS (ESI) m/z=281 (MH⁺).

### Step 7: 3-Bromo-8-isopropyl-6-phenyl-imidazo[1,2-a]pyridine-2-carboxylic Acid

A mixture 8-isopropyl-6-phenyl-imidazo[1,2-a]pyridine-2-carboxylic acid (75 mg, 0.2 mmol) and N-bromosuccinimide (43 mg, 0.24 mmol) was stirred at room temperature in DMF (3 mL) for 12 hours. The mixture was diluted with EtOAc (10 mL) and washed with water (10 mL), 1M sodium thiosulfate solution (10 mL), and brine (10 mL). The filtrate was dried (Na₂SO₄), filtered and concentrated to give 3-bromo-8-isopropyl-6-phenyl-imidazo[1,2-a]pyridine-2-carboxylic acid (60%) as a brown solid. MS (ESI) m/z=361 (MH⁺).

### Step 8: 3-Bromo-8-isopropyl-6-phenyl-imidazo[1,2-a]pyridine-2-carboxylic Acid (Thiophen-2-ylmethyl)-amide (compound 645)

Prepared using standard HATU coupling of the above acid. ¹H NMR (d₆-DMSO, 300 MHz) [symbol] 1.39 (d, 6H, J=6.9 Hz), 3.66 (m, 1H), 4.63 (d, 2H, J=6.6 Hz), 6.95 (m, 1H), 7.03 (m, 1H), 7.37-7.59 (m, 5H), 7.79 (m, 2H), 8.34 (m, 1H), 8.95 (t, 1H, J=6.3 Hz); MS (ESI) m/z=455 (MH⁺).

### Example 546

### [3-(3-Fluoro-phenyl)-pyrrolidin-1-yl]-(8-isopropyl-6-phenyl-imidazo[1,2-a]pyridin-2-yl)-methanone (Compound 646)

Prepared using standard HATU coupling. ¹H NMR (d₆-DMSO, 300 MHz) δ 1.39 (d, 3H, J=7.2 Hz), 1.43 (d, 3H, J=7.2 Hz), 2.07 (m, 1H), 2.40 (m, 1H), 3.56 (m, 3H), 3.78 (m, 0.5H), 3.96 (m, 0.5H), 4.07 (m, 1H), 4.43 (m, 0.5H), 4.46 (m, 0.5H), 7.09 (m, 1H), 7.21 (m, 2H), 7.39 (m, 3H), 7.49 (m, 2H), 7.70 (m, 2H), 8.36 (d, 1H, J=1.8 Hz), 8.78 (m, 1H); MS (ESI) m/z=428 (MH⁺).

### Example 547

### (3-Bromo-8-isopropyl-6-phenyl-imidazo[1,2-a]pyridin-2-yl)-[3-(3-fluoro-phenyl)-pyrrolidin-1-yl]-methanone (Compound 647)

Prepared using standard HATU coupling. ¹H NMR (d₆-DMSO, 300 MHz) δ 1.39 (d, 3H, J=7.2 Hz), 1.43 (d, 3H, J=7.2 Hz), 2.10 (m, 1H), 2.31 (m, 1H), 3.34-3.60 (m, 3H), 3.79 (m, 1H), 3.90 (m, 0.5H), 4.07 (m, 1H), 4.26 (m, 0.5H), 7.06 (m, 1H), 7.24 (m, 2H), 7.38 (m, 2H), 7.49 (m, 3H), 7.77 (m, 2H), 8.37 (m, 1H); MS (ESI) m/z=508 (MH⁺).

### Example 548

### 3-Chloro-6-pyrimidin-5-yl-8-trifluoromethyl-imidazo[1,2-a]pyridine-2-carboxylic Acid(thiophen-2-ylmethyl)-amide (Compound 648)

Prepared using similar procedure as in Example 521 (compound 621). ¹H NMR (d₆-DMSO, 300 MHz) [symbol] 4.65 (d, 2H, J=6.3 Hz), 6.95 (dd, 1H, J=3.6, 5.4 Hz), 7.02 (m, 1H), 7.36 (d, 1H, J=5.1 Hz), 8.36 (s, 1H), 8.95 (t, 1H, J=6.6 Hz), 9.12 (s, 1H), 9.26 (s, 1H), 9.31 (s, 2H); MS 455 (MH⁺).

### Example 549

### 3-Chloro-6-(1-isobutyl-1H-pyrazol-4-yl)-8-trifluoromethyl-imidazo[1,2-a]pyridine-2-carboxylic Acid(thiophen-2-ylmethyl)-amide (Compound 649)

Prepared using similar procedure as in Example 521 (Compound 621).

¹H NMR (d₆-DMSO, 300 MHz) [symbol] 0.82 (d, 6H, J=6.6 Hz), 2.09 (m, 1H), 3.88 (d, 2H, J=6.9 Hz), 4.57 (d, 2H, J=6.0 Hz), 6.90 (d, 1H, J=3.3 Hz), 6.98 (m, 1H), 7.33 (d, 1H, J=4.8 Hz), 8.12 (s, 2H), 8.15 (s, 2H), 8.48 (s, 2H), 8.78 (s, 2H), 8.79 (t, 1H, J=6.6 Hz); MS 483 (MH⁺).

### Example 550

### 2-[6-(furan-3-yl)-8-(trifluoromethyl)imidazo[1,2-a]pyridin-2-yl]-N-(thiophen-2-ylmethyl)acetamide (Compound 650)

### Step 1: (6-Bromo-8-trifluoromethyl-imidazo[1,2-a]pyridin-2-yl)-acetic Acid Ethyl Ester

(6-Bromo-8-trifluoromethyl-imidazo[1,2-a]pyridin-2-yl)-acetic acid ethyl ester was prepared by reacting 5-bromo-3-trifluoromethyl-pyridin-2-ylamine with 4-chloro-3-oxo-butyric acid ethyl. MS (ESI) m/z=352 (MH⁺).

### Step 2: (6-Bromo-8-trifluoromethyl-imidazo[1,2-a]pyridin-2-yl)-acetic Acid

(6-Bromo-8-trifluoromethyl-imidazo[1,2-a]pyridin-2-yl)-acetic acid ethyl ester was saponified using lithium hydroxide to give (6-bromo-8-trifluoromethyl-imidazo[1,2-a]pyridin-2-yl)-acetic acid. MS (ESI) m/z=324 (MH⁺).

### Step 3: (6-Furan-3-yl-8-trifluoromethyl-imidazo[1,2-a]pyridin-2-yl)-acetic Acid

Prepared using standard Suzuki reaction of the above acid. MS (ESI) m/z=311 (MH⁺).

### Step 4: 2-[6-(furan-3-yl)-8-(trifluoromethyl)imidazo[1,2-a]pyridin-2-yl]-N-(thiophen-2-ylmethyl)acetamide

Using standard HATU coupling of the above acid. ¹H NMR (d₆-DMSO, 300 MHz) δ 3.62 (s, 2H), 4.39 (d, 1H, J=6.0 Hz), 6.87 (m, 1H), 6.93 (m, 1H), 6.97 (m, 1H), 7.32 (m, 1H), 7.75 (m, 1H), 7.87 (s, 1H), 7.91 (s, 1H), 8.33 (s, 1H), 8.64 (m, 1H), 9.07 (s, 1H); MS (ESI) m/z=406 (MH⁺).

### Example 551

### 2-(6-Bromo-8-trifluoromethyl-imidazo[1,2-a]pyridin-2-yl)-1-[3-(3-fluoro-phenyl)-pyrrolidin-1-yl]-ethanone (Compound 651)

Prepared using standard HATU coupling of (6-bromo-8-trifluoromethyl-imidazo[1,2-a]pyridin-2-yl)-acetic acid. MS (ESI) m/z=471 (MH⁺).

### Example 552

### 1-[3-(3-fluorophenyl)pyrrolidin-1-yl]-2-[6-(furan-3-yl)-8-(trifluoromethyl)imidazo[1,2-a]pyridin-2-yl]ethanone (Compound 652)

Prepared using Suzuki coupling of 2-(6-bromo-8-trifluoromethyl-imidazo[1,2-a]pyridin-2-yl)-1-[3-(3-fluoro-phenyl)-pyrrolidin-1-yl]-ethanone (compound 651) and 3-furanboronic acid. ¹H NMR (d₆-DMSO, 300 MHz) δ 2.00 (m, 1H), 2.24 (m, 1H), 3.22 (m, 2H), 3.55 (m, 2H), 3.81 (m, 2.5H), 4.10 (m, 0.5H), 6.98 (m, 2H), 7.08 (m, 2H), 7.33 (m, 1H), 7.76 (m, 1H), 7.88 (m, 2H), 8.33 (s, 1H), 9.05 (s, 1H); MS (ESI) m/z=459 (MH⁺).

### Example 553

### 6-Furan-2-yl-2-(3-phenyl-isoxazol-5-yl)-8-trifluoromethyl-imidazo[1,2-a]pyridine (Compound 653)

### Step 1: 6-Bromo-2-(3-phenyl-isoxazol-5-yl)-8-trifluoromethyl-imidazo[1,2-a]pyridine

A mixture of 5-bromo-3-trifluoromethyl-pyridin-2-ylamine (150 mg, 0.622 mmol), and 2-bromo-1-(3-phenylisoxazol-5-yl)ethan-1-one (248 mg, 0.934 mmol) was heated in DMF (1.25 mL) at 50° C. for 1 day. The mixture was then heated at 70° C. for 15 hours. Upon cooling, the mixture was poured into ice-water (20 mL) to give an orange solid which was crystallized from DCM/EtOAc to give the product (72 mg) as orange needles. The residue was purified by silica gel chromatography [n-hex/EtOAc. (4:1 v/v)] to give 6-bromo-2-(3-phenyl-isoxazol-5-yl)-8-trifluoromethyl-imidazo[1,2-a]pyridine as white solid (56 mg). ¹H NMR (d₆-DMSO, 300 MHz) [symbol] 7.50-7.56 (m, 3H), 7.62 (s, 1H), 7.98-8.04 (m, 3H), 8.63 (s, 1H), 9.25 (dd, 1H, J=0.6, 1.8 Hz); MS (ESI) m/z=409.9 (MH⁺).

### Step 2: 6-Furan-2-yl-2-(3-phenyl-isoxazol-5-yl)-8-trifluoromethyl-imidazo[1,2-a]pyridine (Compound 653)

A mixture of 6-bromo-2-(3-phenyl-isoxazol-5-yl)-8-trifluoromethyl-imidazo[1,2-a]pyridine (40 mg, 0.098 mmol), 2-furanbornoic acid (32.9 mg, 0.294 mmol), Pd(PPh₃)₄ (5.7 mg, 0.005 mmol) was heated in aq. K₃PO₄ (1M, 0.5 mL) and 1,4-dioxane (1.5 mL) at 130° C. under standard microwave conditions for 10 min. Upon cooling, the mixture was diluted with EtOAc (30 mL) and washed with saturated aqueous NaHCO₃ (10 mL), then brine (10 mL), dried (Na₂SO₄), filtered and concentrated. The product was crystallized from DCM/EtOAc to give 6-furan-2-yl-2-(3-phenyl-isoxazol-5-yl)-8-trifluoromethyl-imidazo[1,2-a]pyridine (19.7 mg) as white needles. ¹H NMR (d₆-DMSO, 300 MHz) [symbol] 86.68 (dd, 1H, J=1.8, 3.5 Hz), 7.27 (d, 1H, J=2.9 Hz), 7.51-7.55 (m, 3H), 7.59 (s, 1H), 7.87 (dd, 1H, J=0.6, 1.8 Hz), 8.00-8.04 (m, 2H), 8.18 (brs, 1H), 8.73 (s, 1H), 9.23 (s, 1H); MS (ESI) m/z=396.1 (MH⁺).

### Example 554

### 6-Furan-3-yl-2-(3-phenyl-isoxazol-5-yl)-8-trifluoromethyl-imidazo[1,2-a]pyridine (Compound 654)

6-Furan-3-yl-2-(3-phenyl-isoxazol-5-yl)-8-trifluoromethyl-imidazo[1,2-a]pyridine was prepared using similar method as in Example 553 (compound 653) with the use of 3-furanboronic acid. ¹H NMR (d₆-DMSO, 300 MHz) [symbol] 7.06 (dd, 1H, J=0.8, 2 Hz), 7.50-7.55 (m, 3H), 7.56 (s, 1H), 7.84 (t, 1H, J=1.8 Hz), 7.99-8.03 (m, 2H), 8.13 (brs, 1H), 8.45 (s, 1H), 8.61 (s, 1H), 9.18 (s, 1H); MS (ESI) m/z=396.1 (MH⁺).

### Example 555

### 3-Chloro-6-furan-2-yl-2-(3-phenyl-[1,2,4]oxadiazol-5-yl)-8-trifluoromethyl-imidazo[1,2-a]pyridine (Compound 655)

A mixture of 3-chloro-6-furan-3-yl-8-trifluoromethyl-imidazo[1,2-a]pyridine-2-carboxylic acid (100 mg, 0.302 mmol), N′-hydroxybenzenecarboximidamide (49.4 mg, 0.363 mmol), HATU (138 mg, 0.363 mmol), N,N-diisopropylethylamine (158 μL, 0.907 mmol) was stirred in DMF (1.5 mL). After 30 min, the mixture was diluted with EtOAc (25 mL) and washed with HCl (1N, 10 mL), saturated aqueous NaHCO₃ (10 mL), then brine (10 mL). The organic layer was dried (Na₂SO₄), filtered and concentrated to give a film which was dissolved in DMF (6 mL) and heated at 150° C. for 10 min under microwave conditions. Upon cooling, the mixture was diluted with EtOAc (60 mL) and washed with water (30 mL), then brine (20 mL), dried (Na₂SO₄), filtered and concentrated. The crude product was purified by silica gel chromatography [DCM/n-hex/EtOAc (3:3:0.2 v/v)] to give 3-chloro-6-furan-2-yl-2-(3-phenyl-[1,2,4]oxadiazol-5-yl)-8-trifluoromethyl-imidazo[1,2-a]pyridine (22.6 mg) as a white powder. ¹H NMR (d₆-DMSO, 300 MHz) [symbol] 6.72 (dd, 1H, J=1.8, 3.2 Hz), 7.45 (d, 1H, J=3.5 Hz), 7.58-7.68 (m, 3H), 7.91 (d, 1H, J=1.8 Hz), 8.11-8.16 (m, 2H), 8.35 (s, 1H), 8.80 (s, 1H); MS (ESI) m/z=431 (MH⁺).

### Example 556

### 2-(3-Benzyl-[1,2,4]oxadiazol-5-yl)-3-chloro-6-furan-2-yl-8-trifluoromethyl-imidazo[1,2-a]pyridine (Compound 656)

2-(3-Benzyl-[1,2,4]oxadiazol-5-yl)-3-chloro-6-furan-2-yl-8-trifluoromethyl-imidazo[1,2-a]pyridine was prepared using similar method as in Example 555 (compound 655) by replacing N′-hydroxybenzenecarboximidamide with N′-hydroxy-2-phenylethanimidamide. ¹H NMR (d₆-DMSO, 300 MHz) [symbol] 4.24 (s, 2H), 6.71 (dd, 1H, J=1.8, 3.5 Hz), 7.26-7.36 (m, 5H), 7.43 (d, 1H, J=3.2 Hz), 7.89 (d, 1H, J=1.2 Hz), 8.32 (brs, 1H), 8.75 (s, 1H); MS (ESI) m/z=445 (MH⁺).

### Example 557

### 3-Chloro-6-furan-2-yl-2-(3-phenoxymethyl-[1,2,4]oxadiazol-5-yl)-8-trifluoromethyl-imidazo[1,2-a]pyridine (Compound 657)

3-Chloro-6-furan-2-yl-2-(3-phenoxymethyl-[1,2,4]oxadiazol-5-yl)-8-trifluoromethyl-imidazo[1,2-a]pyridine was prepared using similar method as in Example 555 (compound 655) by replacing N′-hydroxybenzenecarboximidamide with N′-hydroxy-2-phenoxyethanimidamide. ¹H NMR (d₆-DMSO, 300 MHz) [symbol] 5.41 (s, 2H), 6.97-7.03 (m, 1H), 7.06-7.11 (m, 2H), 7.30-7.36 (m, 3H), 7.84 (t, 1H, J=1.8 Hz), 8.30 (s, 1H), 8.59 (s, 1H), 8.90 (s, 1H); MS (ESI) m/z=461 (MH⁺).

### Example 558

### 1-[1-(3-Chloro-6-furan-3-yl-8-trifluoromethyl-imidazo[1,2-a]pyridine-2-carbonyl)-azetidin-3-yl]-3-ethyl-urea (Compound 658)

Prepared by the same method as that used in Example 391 using the appropriate carbamoyl chloride or isocyanate. White solid (10 mgs, 23%). MS (ESI) m/z=456.0 (MH⁺).

### Example 559

### 3-(3-Fluoro-phenyl)-pyrrolidin-1-yl]-[3-iodo-6-(1H-pyrazol-4-yl)-8-trifluoromethyl-imidazo[1,2-a]pyridin-2-yl]-methanone (compound 659)

HOAT (1.19 g, 8.77 mmol) and EDC (1.68 g, 8.77 mmol) were added together to a stirring solution of N,N′-diisopropylethylamine (4 mL), 3-iodo-6-(1H-pyrazol-4-yl)-8-trifluoromethyl-imidazo[1,2-a]pyridine-2-carboxylic acid (compound 484, 2.31 g, 5.48 mmol), and 3-(3-fluoro-phenyl)-pyrrolidine (1.10 g, 5.48 mmol) in DMF (27 mL). The reaction stirred at room temperature over night and then water was added. The resulting precipitate was filtered and washed successively with H₂O and diethyl ether. The sample was then chromatographed on silica gel, eluting with methanol in dichloromethane, and 3-(3-fluoro-phenyl)-pyrrolidin-1-yl]-[3-iodo-6-(1H-pyrazol-4-yl)-8-trifluoromethyl-imidazo[1,2-a]pyridin-2-yl]-methanone was obtained (1.43 g, 46%) as a white solid. ¹H NMR (d₆-DMSO, 300 MHz) [symbol] 2.07 (t, 1H, J=10.5 Hz), 2.27-2.36 (m, 1H), 3.43-4.07 (m, 4H), 4.15 (dd, 1H, J=7.3, 11.1 Hz), 7.03-7.25 (m, 4H), 7.32-7.43 (m, 2H), 8.15 (d, 1H, J=7.6 Hz), 8.69 (d, 1H, J=5.3 Hz), 13.17 (s, 1H); MS (ESI) m/z=570.0 (MH⁺).

### BIOLOGICAL EXAMPLES

### Example 1

### Anti-Hepatitis C Activity

Compounds can exhibit anti-hepatitis C activity by inhibiting HCV polymerase, by inhibiting other enzymes needed in the replication cycle, or by other pathways. A number of assays have been published to assess these activities. A general method that assesses the gross increase of HCV virus in culture was disclosed in U.S. Pat. No. 5,738,985 to Miles et al. In vitro assays have been reported in Ferrari et al. Jnl. of Vir., 73:1649-1654, 1999; Ishii et al., Hepatology, 29:1227-1235, 1999; Lohmann et al., Jnl of Bio. Chem., 274:10807-10815, 1999; and Yamashita et al., Jnl. of Bio. Chem., 273:15479-15486, 1998.

### Example 2

### Replicon Assay

A cell line, ET (Huh-lucubineo-ET) is used for screening of compounds for inhibiting HCV RNA dependent RNA polymerase. The ET cell line is stably transfected with RNA transcripts harboring a I₃₈₉ luc-ubi-neo/NS3-3′/ET; replicon with firefly luciferase-ubiquitin-neomycin phosphotransferase fusion protein and EMCV-IRES driven NS3-5B polyprotein containing the cell culture adaptive mutations (E1202G; T1280I; K1846T) (Krieger at al, 2001 and unpublished). The ET cells are grown in DMEM (Dulbeco's Modified Eagle's Medium), supplemented with 10% fetal calf serum, 2 mM Glutamine, Penicillin (100 IU/mL)/Streptomycin (100 μg/mL), 1× nonessential amino acids, and 250 μg/mL G418 (“Geneticin”). They are all available through Life Technologies (Bethesda, Md.). The cells are plated at 0.5−1.0×10⁴ cells/well in the 96 well plates and incubated for 24 hrs before adding test compound. The compounds are added to the cells to achieve a final concentration of 0.1 nM to 50 μm and a final DMSO (dimethylsulfoxide) concentration of 0.5%. Luciferase activity is measured 48-72 hours later by adding a lysis buffer and the substrate (Catalog number Glo-lysis buffer E2661 and Bright-Glo luciferase system E2620 Promega, Madison, Wis.). Cells should not be too confluent during the assay. Percent inhibition of replication data is plotted relative to no compound control. Under the same condition, cytotoxicity of the compounds are determined using cell proliferation reagent, WST-1 (Roche, Germany). The compounds showing antiviral activities, but no significant cytotoxicities are chosen to determine EC₅₀ and TC₅₀. For these determinations, a 10 point, 2-fold serial dilution for each compound was used, which spans a concentration range of 1000 fold. EC₅₀ and similarly TC₅₀ values were calculated by fitting % inhibition at each concentration to the following equation:

% inhibition=100%/[(EC₅₀/[I])^{b}+1]

where b is Hill's coefficient.

% inhibition values at a specific concentration, 10 M for example, can also be derived from the equation above.

In some aspects, when tested, the compounds of Formula (I) will exhibit a % inhibition of at least 80% at 10 μM. In other aspects the % inhibition is at least 50% at 10 μM. In other aspects the % inhibition is at least 10% at 10 μM.

When tested, certain compounds of Table 1, 2, and 3 were found to have the % inhibition values listed in Table 4.

TABLE 4

| Compound | % inhibition at |
|---|---|
| Number | 10 μM |
| 102 | 81.0 |
| 103 | 54.7 |
| 104 | 44.0 |
| 105 | 58.2 |
| 106 | 60.8 |
| 107 | 82.4 |
| 108 | 52.2 |
| 110 | 35.9 |
| 111 | 64.2 |
| 112 | 74.2 |
| 113 | 77.3 |
| 114 | 37.9 |
| 115 | 55.3 |
| 117 | 25.8 |
| 119 | 71.5 |
| 120 | 66.6 |
| 121 | 73.9 |
| 122 | 94.3 |
| 123 | 46.1 |
| 124 | 54.3 |
| 125 | 48.0 |
| 128 | 65.2 |
| 129 | 43.0 |
| 130 | 32.3 |
| 131 | 49.2 |
| 133 | 99.8 |
| 137 | 63.0 |
| 138 | 92.1 |
| 139 | 97.9 |
| 145 | 49.8 |
| 146 | 35.1 |
| 147 | 26.8 |
| 148 | 27.7 |
| 150 | 43.5 |
| 152 | 92.5 |
| 154 | 58.8 |
| 156 | 89.3 |
| 157 | 97.8 |
| 158 | 88.5 |
| 159 | 94.8 |
| 160 | 97.3 |
| 162 | 44.2 |
| 163 | 95.4 |
| 164 | 70.1 |
| 166 | 49.1 |
| 169 | 50.3 |
| 170 | 58.0 |
| 171 | 99.4 |
| 172 | 100.0 |
| 173 | 95.2 |
| 174 | 85.7 |
| 175 | 99.4 |
| 176 | 96.8 |
| 177 | 93.2 |
| 178 | 75.2 |
| 179 | 37.5 |
| 180 | 20.4 |
| 181 | 48.2 |
| 182 | 80.8 |
| 184 | 84.4 |
| 185 | 95.7 |
| 186 | 96.0 |
| 187 | 34.2 |
| 188 | 80.5 |
| 189 | 97.3 |
| 190 | 68.1 |
| 191 | 2.3 |
| 192 | 73.9 |
| 193 | 82.3 |
| 194 | 89.5 |
| 196 | 94.0 |
| 197 | 90.6 |
| 198 | 83.2 |
| 199 | 41.0 |
| 200 | 91.6 |
| 201 | 72.4 |
| 202 | 78.1 |
| 203 | 47.5 |
| 204 | 31.7 |
| 207 | 85.7 |
| 208 | 88.5 |
| 209 | 83.4 |
| 211 | 51.8 |
| 212 | 56.7 |
| 213 | 2.7 |
| 214 | 49.0 |
| 215 | 49.0 |
| 216 | 60.3 |
| 217 | 38.8 |
| 218 | 63.5 |
| 219 | 25.4 |
| 221 | 73.6 |
| 222 | 66.1 |
| 224 | 98.7 |
| 225 | 39.8 |
| 226 | 98.8 |
| 227 | 49.2 |
| 230 | 53.4 |
| 232 | 56.6 |
| 233 | 90.6 |
| 234 | 81.8 |
| 235 | 96.6 |
| 236 | 49.0 |
| 238 | 86.9 |
| 239 | 53.0 |
| 240 | 33.4 |
| 241 | 100.0 |
| 242 | 93.0 |
| 243 | 86.2 |
| 245 | 63.9 |
| 248 | 45.8 |
| 249 | 81.4 |
| 250 | 92.0 |
| 251 | 93.4 |
| 252 | 84.8 |
| 253 | 98.0 |
| 254 | 46.9 |
| 255 | 76.6 |
| 256 | 92.0 |
| 257 | 92.2 |
| 258 | 91.0 |
| 259 | 99.4 |
| 260 | 96.0 |
| 261 | 87.9 |
| 262 | 97.3 |
| 263 | 60.3 |
| 268 | 40.2 |
| 275 | 97.8 |
| 276 | 74.0 |
| 277 | 71.7 |
| 279 | 95.8 |
| 280 | 83.9 |
| 281 | 69.3 |
| 282 | 97.6 |
| 283 | 87.5 |
| 284 | 98.8 |
| 285 | 71.8 |
| 286 | 57.9 |
| 287 | 52.3 |
| 288 | 85.0 |
| 289 | 30.3 |
| 290 | 93.9 |
| 293 | 87.2 |
| 294 | 41.5 |
| 297 | 29.3 |
| 299 | 97.6 |
| 300 | 76.9 |
| 301 | 79.9 |
| 302 | 69.5 |
| 303 | 39.7 |
| 305 | 59.5 |
| 306 | 62.5 |
| 307 | 33.5 |
| 308 | 88.2 |
| 309 | 1.8 |
| 311 | 83.2 |
| 312 | 76.2 |
| 314 | 49.6 |
| 316 | 89.2 |
| 317 | 94.6 |
| 318 | 62.4 |
| 319 | 58.8 |
| 320 | 0.6 |
| 321 | 78.2 |
| 322 | 78.7 |
| 323 | 54.6 |
| 324 | 34.6 |
| 326 | 66.7 |
| 327 | 95.4 |
| 328 | 72.5 |
| 329 | 97.6 |
| 330 | 34.4 |
| 331 | 53.3 |
| 332 | 37.7 |
| 333 | 98.9 |
| 334 | 96.1 |
| 335 | 76.3 |
| 336 | 73.2 |
| 337 | 97.7 |
| 338 | 49.3 |
| 339 | 63.0 |
| 340 | 65.6 |
| 341 | 83.6 |
| 347 | 0.1 |
| 348 | 0.1 |
| 351 | 52.1 |
| 352 | 49.8 |
| 353 | 3.5 |
| 354 | 97.8 |
| 356 | 32.5 |
| 358 | 77.3 |
| 359 | 100.0 |
| 360 | 85.2 |
| 362 | 86.9 |
| 363 | 12.6 |
| 364 | 5.3 |
| 365 | 4.4 |
| 367 | 96.2 |
| 368 | 92.2 |
| 369 | 88.0 |
| 370 | 80.4 |
| 371 | 78.6 |
| 374 | 28.6 |
| 375 | 35.4 |
| 376 | 98.8 |
| 377 | 94.2 |
| 378 | 86.4 |
| 379 | 80.8 |
| 380 | 20.2 |
| 381 | 37.9 |
| 382 | 65.4 |
| 388 | 97.2 |
| 389 | 45.3 |
| 390 | 95.8 |
| 391 | 99.9 |
| 393 | 0.5 |
| 395 | 90.4 |
| 396 | 94.2 |
| 397 | 21.9 |
| 398 | 86.9 |
| 399 | 37.7 |
| 402 | 94.9 |
| 403 | 88.9 |
| 404 | 83.9 |
| 405 | 90.0 |
| 406 | 82.5 |
| 407 | 88.0 |
| 409 | 46.6 |
| 410 | 99.1 |
| 411 | 74.8 |
| 412 | 85.0 |
| 413 | 82.8 |
| 414 | 76.3 |
| 415 | 92.6 |
| 416 | 58.0 |
| 417 | 96.5 |
| 420 | 93.6 |
| 422 | 99.8 |
| 423 | 99.8 |
| 424 | 99.7 |
| 425 | 99.3 |
| 426 | 98.2 |
| 427 | 99.1 |
| 428 | 99.9 |
| 429 | 98.0 |
| 430 | 99.4 |
| 431 | 95.1 |
| 432 | 96.5 |
| 433 | 99.2 |
| 434 | 99.8 |
| 435 | 98.8 |
| 436 | 98.0 |
| 437 | 96.9 |
| 438 | 97.8 |
| 439 | 99.2 |
| 440 | 98.9 |
| 441 | 97.6 |
| 442 | 99.5 |
| 443 | 99.3 |
| 444 | 98.0 |
| 445 | 93.9 |
| 446 | 95.8 |
| 447 | 92.8 |
| 448 | 99.2 |
| 449 | 97.4 |
| 450 | 82.5 |
| 451 | 73.5 |
| 452 | 87.6 |
| 453 | 97.6 |
| 454 | 96.1 |
| 456 | 79.3 |
| 458 | 64.6 |
| 459 | 25.5 |
| 460 | 88.0 |
| 461 | 94.3 |
| 462 | 86.5 |
| 463 | 80.6 |
| 464 | 93.7 |
| 465 | 97.9 |
| 466 | 90.4 |
| 468 | 93.9 |
| 472 | 86.6 |
| 474 | 0.7 |
| 475 | 93.0 |
| 476 | 72.0 |
| 477 | 97.6 |
| 478 | 4.9 |
| 480 | 47.7 |
| 483 | 99.9 |
| 485 | 58.9 |
| 486 | 65.8 |
| 487 | 94.2 |
| 488 | 60.2 |
| 489 | 33.0 |
| 491 | 92.4 |
| 492 | 97.4 |
| 493 | 91.4 |
| 494 | 88.5 |
| 495 | 94.2 |
| 496 | 94.3 |
| 497 | 97.8 |
| 498 | 95.0 |
| 499 | 93.4 |
| 500 | 98.0 |
| 504 | 98.9 |
| 510 | 24.0 |
| 511 | 98.6 |
| 514 | 6.6 |
| 518 | 99.6 |
| 519 | 84.7 |
| 521 | 72.8 |
| 522 | 88.6 |
| 523 | 10.2 |
| 523 | 85.6 |
| 524 | 91.0 |
| 525 | 23.4 |
| 528 | 79.1 |
| 529 | 78.9 |
| 530 | 80.3 |
| 531 | 79.3 |
| 532 | 79.1 |
| 533 | 23.4 |
| 534 | 77.6 |
| 535 | 86.6 |
| 536 | 95.8 |
| 537 | 61.0 |
| 538 | 78.3 |
| 539 | 78.9 |
| 540 | 98.6 |
| 541 | 89.7 |
| 542 | 72.4 |
| 543 | 91.2 |
| 544 | 75.4 |
| 545 | 70.2 |
| 546 | 64.5 |
| 547 | 94.7 |
| 548 | 91.2 |
| 549 | 94.3 |
| 550 | 64.8 |
| 551 | 38.5 |
| 552 | 6.0 |
| 553 | 43.6 |
| 554 | 97.4 |
| 555 | 95.5 |
| 556 | 99.5 |
| 557 | 93.0 |
| 558 | 89.3 |
| 559 | 99.1 |
| 560 | 80.9 |
| 561 | 89.1 |
| 562 | 13.4 |
| 563 | 97.4 |
| 564 | 99.0 |
| 565 | 98.0 |
| 566 | 99.4 |
| 567 | 81.3 |
| 568 | 98.8 |
| 569 | 85.3 |
| 570 | 89.1 |
| 571 | 97.8 |
| 572 | 98.2 |
| 573 | 91.2 |
| 574 | 79.1 |
| 575 | 94.3 |
| 576 | 82.6 |
| 577 | 71.2 |
| 578 | 97.2 |
| 579 | 97.6 |
| 581 | 98.2 |
| 582 | 91.2 |
| 583 | 67.9 |
| 584 | 72.5 |
| 585 | 93.2 |
| 586 | 86.0 |
| 587 | 76.9 |
| 588 | 84.5 |
| 589 | 76.0 |
| 590 | 93.9 |
| 591 | 96.9 |
| 592 | 97.1 |
| 593 | 89.5 |
| 594 | 88.5 |
| 595 | 77.2 |
| 596 | 75.6 |
| 597 | 38.7 |
| 598 | 95.9 |
| 599 | 91.1 |
| 600 | 76.0 |
| 601 | 98.3 |
| 602 | 90.5 |
| 603 | 65.8 |
| 604 | 74.1 |
| 605 | 3.1 |
| 606 | 87.9 |
| 607 | 92.2 |
| 608 | 62.7 |
| 609 | 94.1 |
| 610 | 68.7 |
| 611 | 92.4 |
| 612 | 59.3 |
| 613 | 99.4 |
| 614 | 89.7 |
| 616 | 64.9 |
| 617 | 95.0 |
| 618 | 95.9 |
| 619 | 44.0 |
| 620 | 69.0 |
| 624 | 63.9 |
| 625 | 59.8 |
| 629 | 73.3 |
| 633 | 90.2 |
| 634 | 86.4 |
| 635 | 95.9 |
| 637 | 60.8 |
| 638 | 98.5 |
| 639 | 71.9 |
| 640 | 88.1 |
| 641 | 80.9 |
| 642 | 73.8 |
| 643 | 74.5 |
| 644 | 59.6 |
| 645 | 87.1 |
| 646 | 42.4 |
| 647 | 58.6 |
| 649 | 62.7 |
| 650 | 65.7 |
| 652 | 82.1 |
| 654 | 20.4 |
| 656 | 34.1 |
| 657 | 86.9 |
| 659 | 93.8 |

### Formulation Examples

The following are representative pharmaceutical formulations containing a compound of Formula (I).

### Formulation Example 1

### Tablet Formulation

The following ingredients are mixed intimately and pressed into single scored tablets.

| | Quantity per |
|---|---|
| Ingredient | tablet, mg |
| compound | 400 |
| cornstarch | 50 |
| croscarmellose sodium | 25 |
| lactose | 120 |
| magnesium stearate | 5 |

### Formulation Example 2

### Capsule Formulation

The following ingredients are mixed intimately and loaded into a hard-shell gelatin capsule.

| | Quantity per |
|---|---|
| Ingredient | capsule, mg |
| compound | 200 |
| Lactose, spray-dried | 148 |
| magnesium stearate | 2 |

### Formulation Example 3

### Suspension Formulation

The following ingredients are mixed to form a suspension for oral administration.

| Ingredient | Amount | |
|---|---|---|
| compound | 1.0 | g |
| fumaric acid | 0.5 | g |
| sodium chloride | 2.0 | g |
| methyl paraben | 0.15 | g |
| propyl paraben | 0.05 | g |
| granulated sugar | 25.0 | g |
| sorbitol (70% solution) | 13.00 | g |
| Veegum K (Vanderbilt Co.) | 1.0 | g |
| flavoring | 0.035 | mL |
| colorings | 0.5 | mg |

| | |
|---|---|
| distilled water | q.s. (quantity sufficient) to 100 mL |

### Formulation Example 4

### Injectable Formulation

The following ingredients are mixed to form an injectable formulation.

| Ingredient | Amount |
|---|---|
| compound | 0.2 mg-20 mg |
| sodium acetate buffer solution, | 0.4M 2.0 mL |
| HCl (1N) or NaOH (1N) | q.s. to suitable pH |
| water (distilled, sterile) | q.s. to 20 mL |

### Formulation Example 5

### Suppository Formulation

A suppository of total weight 2.5 g is prepared by mixing the compound with Witepsol® H-15 (triglycerides of saturated vegetable fatty acid; Riches-Nelson, Inc., New York), and has the following composition:

| Ingredient | Amount |
|---|---|
| compound | 500 mg |
| Witepsol ® H-15 | balance |

While some embodiments have been shown and described, various modifications and substitutions may be made thereto without departing from the spirit and scope of the invention: For example, for claim construction purposes, it is not intended that the claims set forth hereinafter be construed in any way narrower than the literal language thereof, and it is thus not intended that exemplarary embodiments from the specification be read into the claims. Accordingly, it is to be understood that the present invention has been described by way of illustration and not limitations on the scope of the claims.

1. At least one chemical entity selected from compounds of Formula 1: 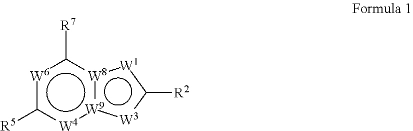
and pharmaceutically acceptable salts thereof, wherein
W¹ is selected from CR¹ and NR¹;
W³ is selected from CR³ and NR³;
W⁴ is selected from CR⁴ and N;
W⁶ is selected from CR⁶ and N;
W⁸ is selected from C and N;
W⁹ is selected from C and N;
R¹ is absent or is selected from hydrogen, halogen, optionally substituted alkyl, optionally substituted alkenyl, optionally substituted alkynyl, optionally substituted cycloalkyl, optionally substituted amino, optionally substituted heterocycloalkyl, optionally substituted aryl, optionally substituted heteroaryl, —OR¹⁵, —SR¹⁵, —S(O)R¹⁶, —S(O)₂R¹⁶, —S(O)₂NR¹⁰R¹¹, —NR¹⁰R¹¹, —NR¹¹C(O)NR¹⁰R¹¹, —NR¹¹C(S)NR¹¹R¹¹, —NR¹¹S(O)₂R¹⁴ —NR¹¹C(O)OR¹³, —NR¹¹C(O)R¹², —C(NR¹¹)NR¹⁰R¹¹, —C(O)NR¹⁰R¹¹, —C(O)OR¹³ —CN, —NO₂, and —C(O)R¹²;
R² is selected from halogen, optionally substituted alkyl, optionally substituted alkenyl, optionally substituted alkynyl, optionally substituted cycloalkyl, optionally substituted amino, optionally substituted heterocycloalkyl, optionally substituted aryl, optionally substituted heteroaryl, —OR¹⁵, —SR¹⁵, —S(O)R¹⁶, —S(O)₂R¹⁶, —S(O)₂NR¹⁰R¹¹, —NR¹⁰R¹¹, —NR¹¹C(O)NR¹⁰R¹¹, —NR¹¹C(S)NR¹⁰R¹¹, —NR¹¹S(O)₂R¹⁴—NR¹¹C(O)OR¹³, —NR¹¹C(O)R¹² —C(NR¹¹)NR¹⁰R¹¹, —C(O)NR¹⁰R¹¹, —C(O)OR¹³, —CN, —NO₂, and —C(O)R¹²;
R³ is absent or is selected from hydrogen, halogen, optionally substituted alkyl, optionally substituted alkenyl, optionally substituted alkynyl, optionally substituted cycloalkyl, optionally substituted amino, optionally substituted heterocycloalkyl, optionally substituted aryl, optionally substituted heteroaryl, —OR¹⁵, —SR¹⁵, —S(O)R¹⁶, —S(O)₂R¹⁶, —S(O)₂NR¹⁰R¹¹, —NR¹⁰R¹¹ —NR¹¹C(O)NR¹⁰R¹¹, —NR¹¹C(S)NR¹⁰R¹¹, —NR¹¹S(O)₂R¹⁴—NR C(O)OR¹³ —NR¹¹C(O)R¹², —C(NR¹¹)NR¹⁰R¹¹, —C(O)NR¹⁰R¹¹, —C(O)OR¹³, —CN, —NO₂, and —C(O)R¹²;
R⁴ is selected from hydrogen, halogen, optionally substituted alkyl, optionally substituted alkenyl, optionally substituted alkynyl, optionally substituted cycloalkyl, optionally substituted amino, optionally substituted heterocycloalkyl, optionally substituted aryl, optionally substituted heteroaryl, —OR¹⁵, —SR¹⁵, —S(O)R¹⁶, —S(O)₂R¹⁶, —S(O)₂NR¹⁰R¹¹, —NR¹¹R¹¹ —NR¹¹C(O)NR¹⁰R¹¹, —NR¹¹C(S)NR¹⁰R¹¹, —NR¹¹S(O)₂R¹⁴—NR¹¹C(O)OR¹³, —NR C(O)R¹², —C(NR¹¹)NR¹⁰R¹¹, —C(O)NR¹⁰R¹¹, —C(O)OR¹³, —CN, —NO₂, and —C(O)R¹²;
R⁵ is selected from halogen, optionally substituted alkyl, optionally substituted alkenyl, optionally substituted alkynyl, optionally substituted cycloalkyl, optionally substituted amino, optionally substituted heterocycloalkyl, optionally substituted aryl, optionally substituted heteroaryl, —OR¹⁵, —SR¹⁵, S(O)R¹⁶, —S(O)₂R¹⁶, —S(O)₂NR¹⁰R¹¹, —NR¹⁰R¹¹, —NR¹¹C(O)NR¹⁰R¹¹, —NR¹¹C(S)NR¹⁰R¹¹, —NR¹¹S(O)₂R¹⁴—NR¹¹C(O)OR¹³, —NR¹¹C(O)R¹² —C(NR¹¹)NR¹⁰R¹¹, —C(O)NR¹⁰R¹¹, —C(O)OR¹³, —CN, —NO₂, and —C(O)R¹²;
R⁶ is selected from hydrogen, halogen, optionally substituted alkyl, optionally substituted alkenyl, optionally substituted alkynyl, optionally substituted cycloalkyl, optionally substituted amino, optionally substituted heterocycloalkyl, optionally substituted aryl, optionally substituted heteroaryl, —OR¹⁵, —SR¹⁵, —S(O)R¹⁶, —S(O)₂R¹⁶, —S(O)₂NR¹⁰R¹¹, —NR¹¹R¹¹ —NR¹¹C(O)NR¹⁰R¹¹, —NR¹¹C(S)NR¹⁰R¹¹, —NR¹¹S(O)₂R¹⁴—NR¹¹C(O)OR¹³, —NR¹¹C(O)R¹² —C(NR¹¹)NR¹⁰R¹¹, —C(O)NR¹¹R¹¹, —C(O)OR¹³, —CN, —NO₂, and —C(O)R¹²;
R⁷ is selected from halogen, optionally substituted alkyl, optionally substituted alkenyl, optionally substituted alkynyl, optionally substituted cycloalkyl, optionally substituted amino, optionally substituted heterocycloalkyl, optionally substituted aryl, optionally substituted heteroaryl, —OR¹⁵, —SR¹⁵, S(O)R¹⁶, —S(O)₂R¹⁶, —S(O)₂NR¹⁰R¹¹, —NR¹⁰R¹¹, —NR¹¹C(O)NR¹⁰R¹¹, —NR¹¹C(S)NR¹⁰R¹¹, —NR¹¹S(O)₂R¹⁴—NR¹¹C(O)OR¹³, —NR¹¹C(O)R¹² —C(NR¹¹)NR¹⁰R¹¹, —C(O)NR¹¹R¹¹, —C(O)OR¹³, —CN, —NO₂, and —C(O)R¹²;
R¹⁰ and R¹¹ are independently selected from hydrogen, optionally substituted alkyl, optionally substituted amino, optionally substituted alkoxy, optionally substituted cycloalkyl, optionally substituted heterocycloalkyl, optionally substituted aryl, and optionally substituted heteroaryl, or R¹⁰ and R¹¹, taken together with any intervening atoms, form a ring system selected from optionally substituted heterocycloalkyl, and optionally substituted heteroaryl;
R¹² is selected from hydrogen, optionally substituted alkyl, optionally substituted cycloalkyl, optionally substituted heterocycloalkyl, optionally substituted aryl, and optionally substituted heteroaryl;
R¹³ is selected from hydrogen, optionally substituted alkyl, optionally substituted cycloalkyl, optionally substituted heterocycloalkyl, optionally substituted aryl, and optionally substituted heteroaryl;
R¹⁴ is selected from optionally substituted alkyl, optionally substituted cycloalkyl, optionally substituted heterocycloalkyl, optionally substituted aryl, and optionally substituted heteroaryl;
R¹⁵ is selected from hydrogen, optionally substituted alkyl, optionally substituted cycloalkyl, optionally substituted heterocycloalkyl, optionally substituted aryl, and optionally substituted heteroaryl; and
R¹⁶ is selected from optionally substituted alkyl, optionally substituted cycloalkyl, optionally substituted heterocycloalkyl, optionally substituted aryl, and optionally substituted heteroaryl;
provided that
if W¹ is NR¹ and W³ is NR³, then R³ is absent;
if W³ is NR³ and W¹ is NR¹, then R¹ is absent;
at least one of W¹, W³, W⁸, and W⁹ is N;
no more than four of W¹, W³, W⁴, W⁶, W⁸, and W⁹ are N; and
if W¹ is N, W⁴ is N, and W⁶ is CR⁶, then W⁸ is not N;
and further provided that the compound of Formula 1 is not
(5-(5-chlorothiophen-2-yl)-7-(trifluoromethyl)pyrazolo[1,5-a]pyridin-2-yl)(3-(3,4-dimethoxyphenyl)-5-(2-hydroxyphenyl)-4,5-dihydro-1H-pyrazol-1-yl)methanone;
(5-(5-chlorothiophen-2-yl)-7-(trifluoromethyl)pyrazolo[1,5-a]pyridin-2-yl)(3-(2,5-dimethylphenyl)-5-(2-hydroxyphenyl)-4,5-dihydro-1H-pyrazol-1-yl)methanone; or
(5-(5-chlorothiophen-2-yl)-7-(trifluoromethyl)pyrazolo[1,5-a]pyridin-2-yl)(3-(3,4-dichlorophenyl)-5-(2-hydroxyphenyl)-4,5-dihydro-1H-pyrazol-1-yl)methanone.

2. At least one chemical entity of claim 1 wherein R⁵ is selected from optionally substituted cycloalkyl, optionally substituted aryl, optionally substituted heteroaryl, and optionally substituted heterocycloalkyl.

3. At least one chemical entity of claim 1 wherein the compound of Formula 1 is selected from the following compounds:

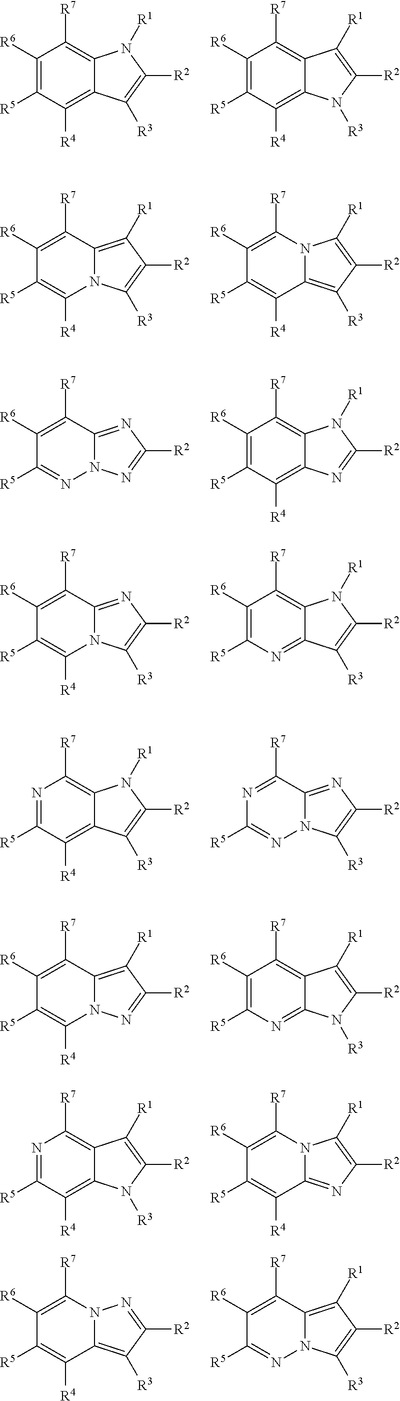

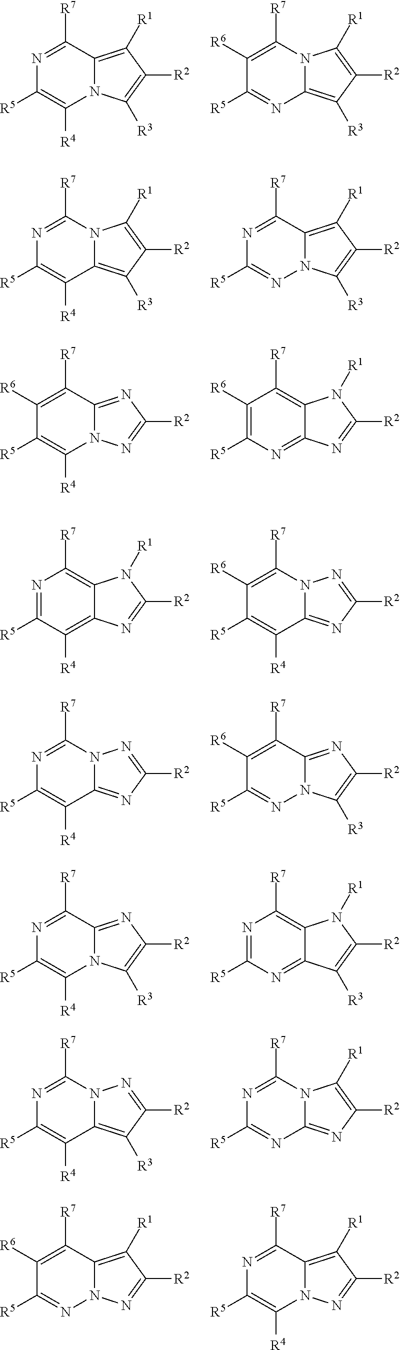

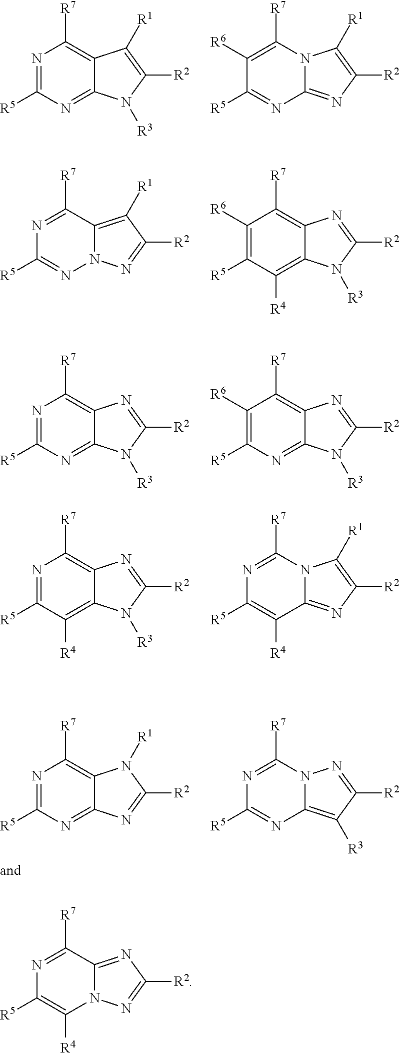

4. At least one chemical entity of claim 3 wherein R⁵ is selected from optionally substituted cycloalkyl, optionally substituted aryl, optionally substituted heteroaryl, and optionally substituted heterocycloalkyl.

5. At least one chemical entity of claim 1 wherein the compound of Formula 1 is selected from the following compounds:

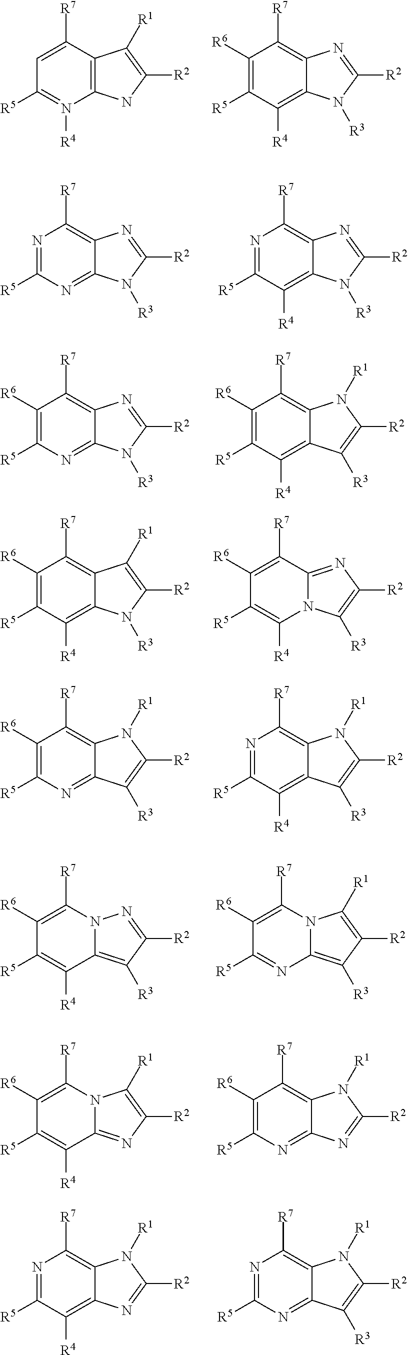

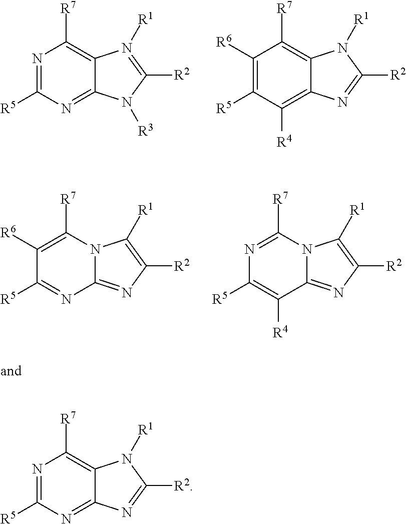

6. At least one chemical entity of claim 5 wherein R⁵ is selected from optionally substituted cycloalkyl, optionally substituted aryl, optionally substituted heteroaryl, and optionally substituted heterocycloalkyl.

7. At least one chemical entity of claim 1 wherein the compound of Formula 1 is selected from the following compounds:

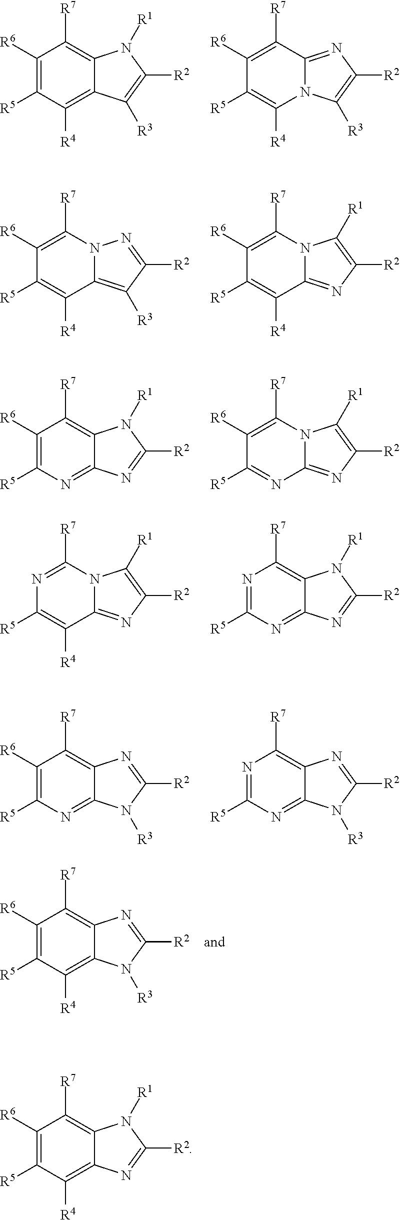

8. At least one chemical entity of claim 7 wherein R⁵ is selected from optionally substituted cycloalkyl, optionally substituted aryl, optionally substituted heteroaryl, and optionally substituted heterocycloalkyl.

9. At least one chemical entity of claim 1 wherein the compound of Formula 1 is selected from the following compounds:

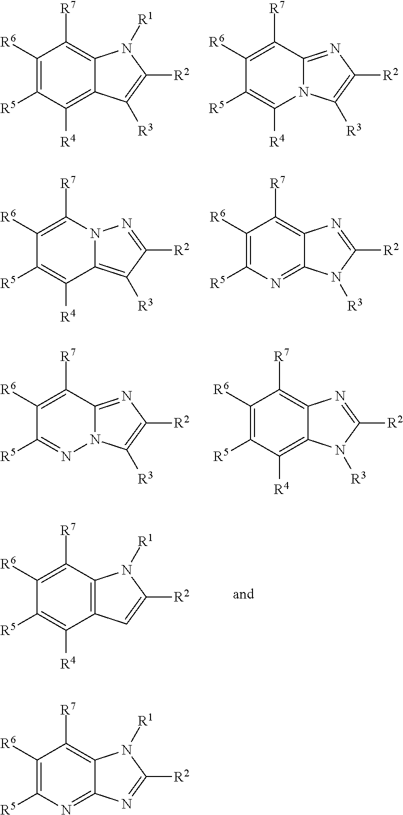

10. At least one chemical entity of claim 9 wherein the compound of Formula 1 is selected from the following compounds:

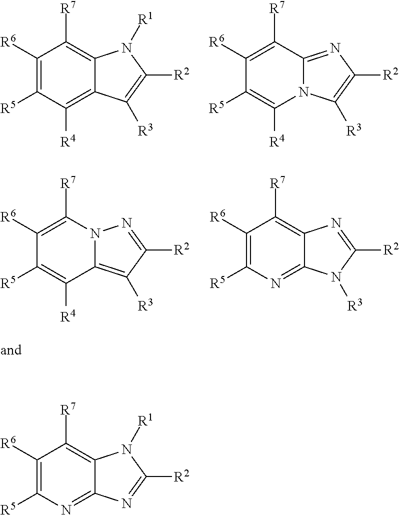

11. At least one chemical entity of claim 10 wherein the compound of Formula 1 is

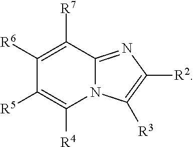

12. At least one chemical entity of claim 9 wherein R⁵ is selected from optionally substituted cycloalkyl, optionally substituted aryl, optionally substituted heteroaryl, and optionally substituted heterocycloalkyl.

13. At least one chemical entity of claim 1 wherein R² is selected from optionally substituted alkyl, —NR¹¹S(O)₂R¹⁴, —NR¹¹C(O)NR¹⁰R¹¹, —NR¹¹C(O)OR¹³—C(O)NR¹⁰R¹¹, and —C(O)OR¹³.

14. At least one chemical entity of claim 13 wherein R² is lower alkyl substituted with —NR¹⁰R¹¹.

15. At least one chemical entity of claim 14 wherein R² is —CH₂—NR¹⁰R¹¹.

16. At least one chemical entity of claim 13 wherein R² is lower alkyl substituted with —C(O)NR¹⁰R¹¹.

17. At least one chemical entity of claim 16 wherein R² is —CH₂—C(O)NR¹⁰R¹¹.

18. At least one chemical entity of claim 13 wherein R² is —C(O)NR¹⁰R¹¹.

19. At least one chemical entity of claim 14 wherein R¹⁰ and R¹¹, together with any intervening atoms, form a substituted 3- to 7-membered nitrogen containing heterocycloalkyl which optionally further includes one or two additional heteroatoms chosen from N, O, S, S(O), S(O)₂, and P(O), wherein said 3- to 7-membered nitrogen containing heterocycloalkyl is substituted with a group —Y—R³⁰ and optionally substituted with a second group R³¹, wherein
Y is a bond or is selected from —NR¹⁰—, —NR¹¹SO₂—, —O—, —S—, —C(O)NR¹⁰—, and —S(O)₂R¹⁰—;
R³⁰ is selected from optionally substituted alkyl, optionally substituted cycloalkyl, optionally substituted heterocycloalkyl, optionally substituted aryl, and optionally substituted heteroaryl; and
R³¹ is selected from halogen, optionally substituted alkyl, optionally substituted alkenyl, optionally substituted alkynyl, optionally substituted cycloalkyl, optionally substituted heterocycloalkyl, optionally substituted aryl, optionally substituted heteroaryl, optionally substituted alkoxy, —OH, —SH, —NO₂, —NR¹⁰R¹¹, —C(O)NR¹⁰R¹¹, —C(O)OR¹³, —SO₂NR¹⁰R¹¹, —NR¹¹C(S)NR¹⁰R¹¹, —NR¹¹C(O)NR¹⁰R¹¹, —CN, —NR¹¹SO₂R¹⁴, and —NR¹¹CO₂R¹³.

20. At least one chemical entity of claim 19 wherein R¹⁰ and R¹¹, together with any intervening atoms, form a substituted 3- to 7-membered nitrogen containing heterocycloalkyl which optionally further includes one or two additional heteroatoms chosen from N, O, S, S(O), S(O)₂, and P(O), wherein said 3- to 7-membered nitrogen containing heterocycloalkyl is substituted with a group —Y—R³⁰ and optionally substituted with a second group R³¹, wherein
Y is a bond or is selected from —O—, —S—, —C(O)NR¹⁰—, and —S(O)₂R¹⁰—;
R³⁰ is selected from optionally substituted cycloalkyl, optionally substituted heterocycloalkyl, optionally substituted aryl, and optionally substituted heteroaryl; and
R³¹ is selected from halogen, optionally substituted alkyl, optionally substituted alkenyl, optionally substituted alkynyl, optionally substituted cycloalkyl, optionally substituted heterocycloalkyl, optionally substituted aryl, optionally substituted heteroaryl, optionally substituted alkoxy, —NO₂, —NR¹⁰R¹¹, —C(O)NR¹⁰R¹¹, —C(O)OR¹³, —SO₂NR¹⁰R¹¹, —NR¹¹C(S)NR¹⁰R¹¹, —NR¹¹C(O)NR¹⁰R¹¹, —CN, —NR¹¹SO₂R¹⁴, and —NR¹¹CO₂R¹³.

21. At least one chemical entity of claim 19 wherein Y is a bond or is selected from —NR¹⁰— and —O—.

22. At least one chemical entity of claim 21 wherein Y is a bond or is —O—.

23. At least one chemical entity of claim 22 wherein Y is a bond.

24. At least one chemical entity of claim 19 wherein R³⁰ is selected from optionally substituted aryl and optionally substituted heteroaryl.

25. At least one chemical entity of claim 24 wherein R³⁰ is selected from phenyl, thiophen-2-yl, thiophen-3-yl, furan-2-yl, furan-3-yl, thiazol-2-yl, thiazol-4-yl, thiazol-5-yl, pyrazol-4-yl, imidazol-4-yl, and imidazol-2-yl.

26. At least one chemical entity of claim 25 wherein R³⁰ is selected from phenyl, thiophen-2-yl, thiophen-3-yl, furan-2-yl, and furan-3-yl.

27. At least one chemical entity of claim 19 wherein R¹⁰ and R¹¹, together with any intervening atoms, form a pyrrolidinyl, piperidinyl, piperazinyl, 5,6-dihydropyridin-1(2H)-yl, 4,5-dihydro-1H-pyrazol-1-yl, 2,5-dihydro-1H-pyrrol-1-yl, or azetidinyl ring.

28. At least one chemical entity of claim 14 wherein R¹¹ is selected from lower alkyl and hydrogen.

29. At least one chemical entity of claim 14 wherein R¹⁰ is selected from optionally substituted alkyl, optionally substituted cycloalkyl, optionally substituted heterocycloalkyl, and optionally substituted aryl.

30. At least one chemical entity of claim 29 wherein R¹⁰ is —(CR¹⁷R¹⁸)ₙR¹⁹,
wherein R¹⁷ and R¹⁸ are independently selected from hydrogen, carboxy, optionally substituted aminocarbonyl, lower carboxy ester, and lower alkyl; n is 0, 1 or 2; and R¹⁹ is chosen from optionally substituted aryl and optionally substituted heteroaryl.

31. At least one chemical entity of claim 30 wherein R¹⁰ is benzyl, thiophen-2-yl-ethyl, thiophen-3-yl-methyl, furan-2-yl-methyl, and furan-3-yl-methyl, each of which is optionally substituted.

32. At least one chemical entity of claim 1 wherein R² is optionally substituted heteroaryl.

33. At least one chemical entity of claim 32 wherein R² is isoxazol-5-yl or [1,2,4]oxadiazol-5-yl, each of which is optionally substituted.

34. At least one chemical entity of claim 33 wherein R² is isoxazol-5-yl or [1,2,4]oxadiazol-5-yl, each of which is optionally substituted with a group chosen from optionally substituted aryl and optionally substituted alkyl.

35. At least one chemical entity of claim 1 wherein, if present, R³ is selected from optionally substituted alkyl and halogen.

36. At least one chemical entity of claim 35 wherein R³ is selected from lower alkyl and halogen.

37. At least one chemical entity of claim 36 wherein R³ is halogen.

38. At least one chemical entity of claim 37 wherein R³ is selected from chlorine and bromine.

39. At least one chemical entity of claim 38 wherein R³ is chlorine.

40. At least one chemical entity of claim 1 wherein R⁴ is selected from hydrogen, optionally substituted alkyl, —NR¹¹SO₂R¹⁴, —NR¹¹C(O)NR¹⁰R¹¹, —NR¹¹CO₂R¹³—S(O)NR¹⁰R¹¹, —NR¹¹C(O)NR¹⁰R¹¹, —CN, —NO₂, and —C(O)R¹².

41. At least one chemical entity of claim 40 wherein R⁴ is selected from hydrogen and optionally substituted lower alkyl.

42. At least one chemical entity of claim 41 wherein R⁴ is hydrogen.

43. At least one chemical entity of claim 1 wherein R⁵ is selected from optionally substituted cycloalkyl, optionally substituted heterocycloalkyl, optionally substituted aryl, and optionally substituted heteroaryl.

44. At least one chemical entity of claim 43 wherein R⁵ is selected from optionally substituted aryl and optionally substituted heteroaryl.

45. At least one chemical entity of claim 44 wherein R⁵ is selected from pyrid-3-yl, pyrazol-4-yl, phenyl, furan-2-yl, furan-3-yl, thiophen-2-yl, and thiophen-3-yl, each of which is optionally substituted.

46. At least one chemical entity of claim 45 wherein R⁵ is selected from phenyl, furan-2-yl, furan-3-yl, thiophen-2-yl, and thiophen-3-yl, each of which is optionally substituted.

47. At least one chemical entity of claim 1 wherein R⁶ is selected from hydrogen, halogen, optionally substituted alkyl, —OR¹⁵, —S(O)NR¹⁰R¹¹, —C(O)R¹², —NO₂, —C(O)NR¹⁰R¹¹, and —NR¹⁰R¹¹.

48. At least one chemical entity of claim 47 wherein R⁶ is selected from hydrogen, halogen, optionally substituted alkyl, —S(O)NR¹⁰R¹¹, —C(O)R¹², —NO₂, —C(O)NR¹⁰R¹¹, and —NR¹⁰R¹¹.

49. At least one chemical entity of claim 48 wherein R¹¹ is hydrogen.

50. At least one chemical entity of claim 47 wherein R¹⁰ is selected from optionally substituted alkyl and optionally substituted cycloalkyl.

51. At least one chemical entity of claim 50 wherein R¹⁰ and R¹¹, taken together with any intervening atoms, form an optionally substituted heterocycloalkyl ring.

52. At least one chemical entity of claim 48 wherein R⁶ is selected from hydrogen, halogen, and optionally substituted alkyl.

53. At least one chemical entity of claim 52 wherein R⁶ is selected from hydrogen and halogen.

54. At least one chemical entity of claim 53 wherein R⁶ is hydrogen.

55. At least one chemical entity of claim 1 wherein R⁷ is selected from halogen, optionally substituted alkyl, optionally substituted cycloalkyl, optionally substituted alkoxy, heterocycloalkyl, optionally substituted aryl, —SO₂NR¹⁰R¹¹, and —NR¹⁰R¹¹.

56. At least one chemical entity of claim 55 wherein R⁷ is selected from halogen, optionally substituted alkyl, optionally substituted cycloalkyl, optionally substituted alkoxy, heterocycloalkyl, optionally substituted aryl, and —NR¹⁰R¹¹.

57. At least one chemical entity of claim 56 wherein R⁷ is selected from optionally substituted alkyl, optionally substituted cycloalkyl, optionally substituted alkoxy, and —NR¹⁰R¹¹.

58. At least one chemical entity of claim 57 wherein R⁷ is selected from optionally substituted alkyl, optionally substituted alkoxy, and —NR¹⁰R¹¹.

59. At least one chemical entity of claim 58 wherein R⁷ is selected from optionally substituted lower alkoxy and optionally substituted lower alkyl.

60. At least one chemical entity of claim 59 wherein R⁷ is polyhalogenated lower alkoxy.

61. At least one chemical entity of claim 60 wherein R⁷ selected from trifluoromethoxy and difluorochloromethoxy.

62. At least one chemical entity of claim 59 wherein R⁷ is polyhalogenated lower alkyl.

63. At least one chemical entity of claim 62 wherein R⁷ is polyhalogenated methyl.

64. At least one chemical entity of claim 63 wherein R⁷ is selected from trifluoromethyl and difluorochloromethyl.

65. At least one chemical entity of claim 64 wherein R⁷ is trifluoromethyl.

66. At least one chemical entity of claim 58 wherein R⁷ is —NR¹⁰R¹¹.

67. At least one chemical entity of claim 66 wherein R¹¹ is hydrogen.

68. At least one chemical entity of claim 66 wherein R¹⁰ is optionally substituted lower alkyl.

69. (canceled)

70. A pharmaceutical composition comprising a pharmaceutically acceptable diluent and a therapeutically effective amount of at least one chemical entity of claim 1.

71. A pharmaceutical composition comprising a pharmaceutically acceptable diluent and a therapeutically effective amount of at least one chemical entity chosen from compounds of Formula 1a 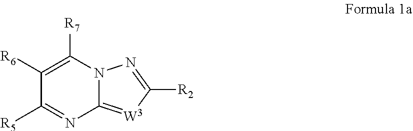
and pharmaceutically acceptable salts thereof, wherein
W³ is selected from CR³ and NR³;
R² is selected from halogen, optionally substituted alkyl, optionally substituted alkenyl, optionally substituted alkynyl, optionally substituted cycloalkyl, optionally substituted amino, optionally substituted heterocycloalkyl, optionally substituted aryl, optionally substituted heteroaryl, —OR¹⁵, —SR¹⁵, —S(O)R¹⁶, —S(O)₂R¹⁶, —S(O)₂NR¹⁰R¹¹, —NR¹⁰R¹¹, —NR¹¹C(O)NR¹⁰R¹¹—NR¹¹C(S)NR¹⁰R¹¹, —NR¹¹S(O)₂R¹⁴—NR¹¹C(O)OR¹³, —NR¹¹C(O)R¹² —C(NR¹¹)NR¹⁰R¹¹, —C(O)NR¹⁰R¹¹, —C(O)OR¹³, —CN, —NO₂, and —C(O)R¹²;
R³ is absent or is selected from halogen, optionally substituted alkenyl, optionally substituted alkynyl, optionally substituted cycloalkyl, optionally substituted amino, optionally substituted heterocycloalkyl, optionally substituted aryl, optionally substituted heteroaryl, —OR¹⁵, —SR¹⁵, —S(O)R¹⁶, —S(O)₂R¹⁶, —S(O)₂NR¹¹R¹¹, —NR¹⁰R¹¹, —NR¹¹C(O)NR¹⁰R¹¹, —NR¹¹C(S)NR¹⁰R¹¹, —NR¹¹S(O)₂R¹⁴—NR¹¹C(O)OR¹³, —NR¹¹C(O)R¹², —C(NR¹¹)NR¹⁰R¹¹, —C(O)NR¹⁰R¹¹, —C(O)OR¹³, —CN, —NO₂, and —C(O)R¹²;
R⁵ is selected from halogen, optionally substituted alkyl, optionally substituted alkenyl, optionally substituted alkynyl, optionally substituted cycloalkyl, optionally substituted amino, optionally substituted heterocycloalkyl, optionally substituted aryl, optionally substituted heteroaryl, —OR¹⁵, —SR¹⁵, —S(O)R¹⁶, —S(O)₂R¹⁶, —S(O)₂NR¹⁰R¹¹, —NR¹⁰R¹¹, —NR¹¹C(O)NR¹⁰R¹¹, —NR¹¹C(S)NR¹⁰R¹¹, —NR¹¹S(O)₂R¹⁴—NR¹¹C(O)OR¹³, —NR¹¹C(O)R¹², —C(NR¹¹)NR¹⁰R¹¹, —C(O)NR¹⁰R¹¹, —C(O)OR¹³, —CN, —NO₂, and —C(O)R¹²;
R⁶ is selected from hydrogen, halogen, optionally substituted alkyl, optionally substituted alkenyl, optionally substituted alkynyl, optionally substituted cycloalkyl, optionally substituted amino, optionally substituted heterocycloalkyl, optionally substituted aryl, optionally substituted heteroaryl, —OR¹⁵, —SR¹⁵, —S(O)R¹⁶, —S(O)₂R¹⁶, —S(O)₂NR¹⁰R¹¹, —NR¹⁰R¹¹, —NR¹¹C(O)NR¹⁰R¹¹, —NR¹¹C(S)NR¹⁰R¹¹, —NR¹¹S(O⁾₂R¹⁴—NR¹¹C(O)OR¹³, —NR C(O)R¹², —C(NR¹¹)NR¹⁰R¹¹, —C(O)NR¹⁰R¹¹, —C(O)OR¹³, —CN, —NO₂, and —C(O)R¹²;
R⁷ is selected from halogen, optionally substituted alkyl, optionally substituted alkenyl, optionally substituted alkynyl, optionally substituted cycloalkyl, optionally substituted amino, optionally substituted heterocycloalkyl, optionally substituted aryl, optionally substituted heteroaryl, —OR¹⁵, —SR¹⁵, —S(O)R¹⁶, —S(O)₂R¹⁶, —S(O)₂NR¹⁰R¹¹, —NR¹⁰R¹¹, —NR¹¹C(O)NR¹⁰R¹¹—NR¹¹C(S)NR¹⁰R¹¹, —NR¹¹S(O⁾₂R¹⁴—NR¹¹C(O)OR¹³, —NR¹¹C(O)R¹² —C(NR¹¹)NR¹⁰R¹¹, —C(O)NR¹¹R¹¹, —C(O)OR¹³, —CN, —NO₂, and —C(O)R¹²;
R¹⁰ and R¹¹ are independently selected from hydrogen, optionally substituted alkyl, optionally substituted amino, optionally substituted alkoxy, optionally substituted cycloalkyl, optionally substituted heterocycloalkyl, optionally substituted aryl, and optionally substituted heteroaryl, or R¹⁰ and R¹¹, taken together with any intervening atoms, form a ring system selected from optionally substituted heterocycloalkyl, and optionally substituted heteroaryl;
R¹² is selected from hydrogen, optionally substituted alkyl, optionally substituted cycloalkyl, optionally substituted heterocycloalkyl, optionally substituted aryl, and optionally substituted heteroaryl;
R¹³ is selected from hydrogen, optionally substituted alkyl, optionally substituted cycloalkyl, optionally substituted heterocycloalkyl, optionally substituted aryl, and optionally substituted heteroaryl;
R¹⁴ is selected from optionally substituted alkyl, optionally substituted cycloalkyl, optionally substituted heterocycloalkyl, optionally substituted aryl, and optionally substituted heteroaryl;
R¹⁵ is selected from hydrogen, optionally substituted alkyl, optionally substituted cycloalkyl, optionally substituted heterocycloalkyl, optionally substituted aryl, and optionally substituted heteroaryl; and
R¹⁶ is selected from optionally substituted alkyl, optionally substituted cycloalkyl, optionally substituted heterocycloalkyl, optionally substituted aryl, and optionally substituted heteroaryl.

72. The pharmaceutical composition of claim 71 wherein R² is selected from optionally substituted alkyl, —NR¹¹S(O)₂R¹⁴, —NR¹¹C(O)NR¹⁰R¹¹, —NR¹¹C(O)OR¹³—C(O)NR¹⁰R¹¹, and —C(O)OR¹³.

73. The pharmaceutical composition of claim 72 wherein R² is —C(O)NR¹⁰R¹¹.

74. The pharmaceutical composition of claim 73 wherein R¹¹ is selected from lower alkyl and hydrogen.

75. The pharmaceutical composition of claim 73 wherein R¹⁰ is selected from optionally substituted alkyl, optionally substituted cycloalkyl, optionally substituted heterocycloalkyl, and optionally substituted aryl.

76. The pharmaceutical composition of claim 75 wherein R¹⁰ is —(CR¹⁷R¹⁸)ₙR¹⁹,
wherein R¹⁷ and R¹⁸ are independently selected from hydrogen, carboxy, optionally substituted aminocarbonyl, lower carboxy ester, and lower alkyl; n is 0, 1 or 2; and R¹⁹ is chosen from optionally substituted aryl and optionally substituted heteroaryl.

77. The pharmaceutical composition of claim 76 wherein R¹⁰ is benzyl, thiophen-2-yl-ethyl, thiophen-3-yl-methyl, furan-2-yl-methyl, and furan-3-yl-methyl, each of which is optionally substituted.

78. The pharmaceutical composition of claim 73 wherein R¹⁰ and R¹¹, together with any intervening atoms, form an optionally substituted heterocycloalkyl.

79. The pharmaceutical composition of claim 78 wherein R¹⁰ and R¹¹, together with any intervening atoms, form a substituted 3- to 7-membered nitrogen containing heterocycloalkyl which optionally further includes one or two additional heteroatoms chosen from N, O, S, S(O), S(O)₂, and P(O), wherein said 3- to 7-membered nitrogen containing heterocycloalkyl is substituted with a group —Y—R³⁰ and optionally substituted with a second group R³¹, wherein
Y is a bond or is selected from —NR¹⁰—, —NR¹¹SO₂—, —O—, —S—, —C(O)NR¹⁰—, and —S(O)₂R¹⁰—;
R³⁰ is selected from optionally substituted alkyl, optionally substituted cycloalkyl, optionally substituted heterocycloalkyl, optionally substituted aryl, and optionally substituted heteroaryl; and
R³¹ is selected from halogen, optionally substituted alkyl, optionally substituted alkenyl, optionally substituted alkynyl, optionally substituted cycloalkyl, optionally substituted heterocycloalkyl, optionally substituted aryl, optionally substituted heteroaryl, optionally substituted alkoxy, —OH, —SH, —NO₂, —NR¹⁰R¹¹, —C(O)NR¹⁰R¹¹, —C(O)OR¹³, —SO₂NR¹⁰R¹¹, —NR¹¹C(S)NR¹⁰R¹¹, —NR¹¹C(O)NR¹⁰R¹¹, —CN, —NR¹¹SO₂R¹⁴, and —NR¹¹CO₂R¹³.

80. The pharmaceutical composition of claim 79 wherein Y is a bond or is selected from —NR¹⁰— and —O—.

81. The pharmaceutical composition of claim 80 wherein Y is a bond.

82. The pharmaceutical composition of claim 79 wherein R³⁰ is selected from optionally substituted aryl and optionally substituted heteroaryl.

83. The pharmaceutical composition of claim 82 wherein R³⁰ is selected from phenyl, thiophen-2-yl, thiophen-3-yl, furan-2-yl, and furan-3-yl.

84. The pharmaceutical composition of claim 83 wherein R³⁰ is phenyl.

85. The pharmaceutical composition of claim 71 wherein R³ is halogen.

86. The pharmaceutical composition of claim 85 wherein R³ is selected from chlorine and bromine.

87. The pharmaceutical composition of claim 86 wherein R³ is chlorine.

88. The pharmaceutical composition of claims 71 wherein R⁵ is selected from optionally substituted cycloalkyl, optionally substituted aryl, optionally substituted heteroaryl, and optionally substituted heterocycloalkyl.

89. The pharmaceutical composition of claim 88 wherein R⁵ is selected from optionally substituted cycloalkyl, optionally substituted aryl, and optionally substituted heteroaryl.

90. The pharmaceutical composition of claim 89 wherein R⁵ is selected from optionally substituted aryl and optionally substituted heteroaryl.

91. The pharmaceutical composition of claim 90 wherein R⁵ is selected from phenyl, furan-2-yl, furan-3-yl, thiophen-2-yl, and thiophen-3-yl, each of which is optionally substituted.

92. The pharmaceutical composition of claim 91 wherein R⁵ is selected from phenyl, furan-2-yl, furan-3-yl, thiophen-2-yl, and thiophen-3-yl, each of which is optionally substituted with one or two groups chosen from lower alkyl, halogen, morpholinyl, trifluoromethyl, and lower alkoxy.

93. The pharmaceutical composition of claim 92 wherein R⁵ is selected from phenyl, 3-fluorophenyl, furan-2-yl, furan-3-yl, thiophen-2-yl, and thiophen-3-yl.

94. The pharmaceutical composition of claim 71 wherein R⁶ is selected from hydrogen, halogen, optionally substituted alkyl, —S(O)NR¹⁰R¹¹, —C(O)R¹², —NO₂, —C(O)NR¹⁰R¹¹, and —NR¹⁰R¹¹.

95. The pharmaceutical composition of claim 94 wherein R¹¹ is hydrogen.

96. The pharmaceutical composition of claim 94 wherein R¹⁰ is selected from optionally substituted alkyl and optionally substituted cycloalkyl.

97. The pharmaceutical composition of claim 94 wherein R¹⁰ and R¹¹, taken together with any intervening atoms, form an optionally substituted heterocycloalkyl ring.

98. The pharmaceutical composition of claim 94 wherein R⁶ is selected from hydrogen, halogen, and optionally substituted alkyl.

99. The pharmaceutical composition of claim 98 wherein R⁶ is selected from hydrogen and halogen.

100. The pharmaceutical composition of claim 99 wherein R⁶ is hydrogen.

101. The pharmaceutical composition of claim 71 wherein R⁷ is selected from halogen, optionally substituted alkyl, optionally substituted cycloalkyl, optionally substituted alkoxy, heterocycloalkyl, optionally substituted aryl, and —NR¹⁰R¹¹.

102. The pharmaceutical composition of claim 101 wherein R⁷ is selected from optionally substituted alkyl, optionally substituted cycloalkyl, optionally substituted alkoxy, and —NR¹⁰R¹¹.

103. The pharmaceutical composition of claim 102 wherein R⁷ is selected from optionally substituted alkyl, optionally substituted alkoxy, and —NR¹⁰R¹¹.

104. The pharmaceutical composition of claim 103 wherein R⁷ is selected from optionally substituted lower alkoxy and optionally substituted lower alkyl.

105. The pharmaceutical composition of claim 104 wherein R⁷ is polyhalogenated lower alkoxy.

106. The pharmaceutical composition of claim 105 wherein R⁷ selected from trifluoromethoxy and difluorochloromethoxy.

107. The pharmaceutical composition of claim 104 wherein R⁷ is polyhalogenated lower alkyl.

108. The pharmaceutical composition of claim 107 wherein R⁷ is polyhalogenated methyl.

109. The pharmaceutical composition of claim 108 wherein R⁷ is selected from trifluoromethyl and difluorochloromethyl.

110. The pharmaceutical composition of claim 109 wherein R⁷ is trifluoromethyl.

111. The pharmaceutical composition of claim 103 wherein R⁷ is —NR¹⁰R¹¹.

112. The pharmaceutical composition of claim 111 wherein R¹¹ is hydrogen.

113. The pharmaceutical composition of claim 111 wherein R¹⁰ is optionally substituted lower alkyl.

114. The pharmaceutical composition of claim 113 wherein R¹⁰ is methyl.

115. The pharmaceutical composition of claim 113 wherein R¹⁰ is 2-hydroxyethyl.

116. A pharmaceutical composition comprising a pharmaceutically acceptable diluent and a therapeutically effective amount of at least one chemical entity chosen from
(5-(5-chlorothiophen-2-yl)-7-(trifluoromethyl)pyrazolo[1,5-a]pyridin-2-yl)(3-(3,4-dimethoxyphenyl)-5-(2-hydroxyphenyl)-4,5-dihydro-1H-pyrazol-1-yl)methanone;
(5-(5-chlorothiophen-2-yl)-7-(trifluoromethyl)pyrazolo[1,5-a]pyridin-2-yl)(3-(2,5-dimethylphenyl)-5-(2-hydroxyphenyl)-4,5-dihydro-1H-pyrazol-1-yl)methanone; and
(5-(5-chlorothiophen-2-yl)-7-(trifluoromethyl)pyrazolo[1,5-a]pyridin-2-yl)(3-(3,4-dichlorophenyl)-5-(2-hydroxyphenyl)-4,5-dihydro-1H-pyrazol-1-yl)methanone,
and pharmaceutically acceptable salts thereof.

117. A method for treating a viral infection in a mammal mediated at least in part by a virus in the flaviviridae family of viruses which method comprises administering to a mammal, that has been diagnosed with said viral infection or is at risk of developing said viral infection, a pharmaceutical composition according to claim 70, provided that at least one of R¹ and R³ is halogen.

118. The method according to claim 117, wherein said virus is hepatitis C virus.

119. The method of claim 118 in combination with the administration of a therapeutically effective amount of one or more agents active against hepatitis C virus.

120. The method of claim 119 wherein said agent active against hepatitis C virus is an inhibitor of HCV proteases, HCV polymerase, HCV helicase, HCV NS4B protein, HCV entry, HCV assembly, HCV egress, HCV replicase, HCV NS5A protein, or inosine 5′-monophosphate dehydrogenase.

121. The method of claim 120 wherein said agent active against hepatitis C virus is interferon.
